SEQUENCE LISTING (5 sequences)

SEQ ID NO: 1 - protein, 411 aa, Homo sapiens
ETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLE

SEQ ID NO: 2 - protein, 435 aa, Homo sapiens
TQHGIRLPLRSGLGGAPLGLRLPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQTDESTLE

SEQ ID NO: 3 - protein, 12 aa, Artificial Sequence
  cleavage sequence
KSEVNLDAEFRK

SEQ ID NO: 4 - protein, 403 aa, Homo sapiens
DEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQT

SEQ ID NO: 5 - protein, 408 aa, Homo sapiens
LPRETDEEPEEPGRRGSFVEMVDNLRGKSGQGYYVEMTVGSPPQTLNILVDTGSSNFAVGAAPHPFLHRYYQRQLSSTYRDLRKGVYVPYTQGKWEGELGTDLVSIPHGPNVTVRANIAAITESDKFFINGSNWEGILGLAYAEIARPDDSLEPFFDSLVKQTHVPNLFSLQLCGAGFPLNQSEVLASVGGSMIIGGIDHSLYTGSLWYTPIRREWYYEVIIVRVEINGQDLKMDCKEYNYDKSIVDSGTTNLRLPKKVFEAAVKSIKAASSTEKFPDGFWLGEQLVCWQAGTTPWNIFPVISLYLMGEVTNQSFRITILPQQYLRPVEDVATSQDDCYKFAISQSSTGTVMGAVIMEGFYVVFDRARKRIGFAVSACHVHDEFRTAAVEGPFVTLDMEDCGYNIPQT

(19) United States
(12) Patent Application Publication
(10) Pub. No.: US 2004/0014194 A1
(43) Pub. Date: Jan. 22, 2004

(54) Beta-secretase crystals and methods for preparing and using the same

(75) Inventors: Brian M. Beyer, Lincroft, NJ (US); Gerald S. Hammond, Newark, NJ (US); Paul Reichert, Montville, NJ (US); Corey Strickland, Martinsville, NJ (US); Wenyan Wang, Edison, NJ (US); Patricia C. Weber, Yardley, PA (US); Gwendolyn Tse Wong, Westfield, NJ (US); Lili Zhang, Scotch Plains, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/400,273

(22) Filed: Mar. 26, 2003

Related U.S. Application Data
(60) Provisional application No. 60/367,937, filed on Mar. 27, 2002.

(51) Int. Cl. C12N 9/64; G06F 19/00; G01N 33/48; G01N 33/50

(52) U.S. Cl. 435/226; 702/19

(57) ABSTRACT

The present invention relates to the expression, purification and crystallization of glycosylated β-secretase protein and a complex thereof. The crystals of the invention are useful, inter alia, for determining the three-dimensional structure of β-secretase and of other, related proteins.

This application claims the benefit of U.S. Provisional Patent Application No. 60/367,937, filed Mar. 27, 2002, now pending, which is herein incorporated by reference in its entirety.

### FIELD OF THE INVENTION

This invention relates to crystalline β-secretase (BACE), the three-dimensional structure of BACE, methods for preparing the crystal and the use of the crystal to solve the structure of BACE homologues, mutants, other BACE crystal forms and similar molecules of unknown structure, and the use of BACE crystals to design inhibitors against BACE.

### BACKGROUND OF THE INVENTION

Alzheimer's disease (AD) is a neurodegenerative disease characterized by neuronal loss due to the extracellular accumulation of amyloid plaques and intracellular accumulation of neurofibrillary tangles in the brain (reviewed by Selkoe, D. J. (1999) Nature 399: A23-31). Two major components of amyloid plaques are small peptide fragments Aβ140 and Aβ42, which are generated from cleavage of the membrane-anchored amyloid precursor protein (APP) by the proteolytic activity of β- and γ- secretases. APP is a type I integral membrane protein containing the Aβ segment, which begins at D672 in the longest isoform and spans the boundary of the exocytoplasmic region (28 amino acids) and the transmembrane domain (12-14 amino acids). The γ-secretase activity cleaves APP within the transmembrane domain to produce the carboxy-terminal end of Aβ polypeptide. The β-secretase activity (aspartic protease activity), identified in a protein that is known as “mamapsin 2”, “human β-site APP-cleaving enzyme” or “BACE”, and “Asp 2”, cleaves APP on the extracellular side of the membrane to produce the amino-terminal end of Aβ. (Vassar, R. et al., (1999) Science 286,735, Sinha, S. et al., (1999) Nature 402,537, Yan, R. et al., (1999) Nature 402,522, Hussain, I. et al., (1999) Mol. Cell Neurosci. 14, 419 and Lin, X. (2000) et al., Proc. Natl. Acad. Sci. USA 97, 1456. Another enzyme, known as α-secretase, cleaves APP at a position within the Aβ sequence to produce a soluble APPα (Esch et al., (1990) Science 248: 1122-1124).

During the course of AD, Aβ polypeptide accumulates extracellularly in the brain, and forms large, insoluble amyloid fibrils that elicit both cytotoxic and inflammatory responses. Thus, BACE and γ-secretase proteases are targets for potential inhibitor drugs against AD. Since it was discovered that the β-secretase activity is the rate-limiting step in AP production in vivo (Sinha and Lieberburg, (1999) Proc. Natl. Acad. Sci. USA 96: 11049), BACE has become a prime target for the development of inhibitors to treat AD.

The BACE gene encodes a 501 residue polypeptide having (from N- to C- terminus) an N-terminal signal sequence of 21 amino acids, a pro-protein domain of 22 to 45 residues, which is proteolytically removed by furin to generate a mature β-secretase (Creemers, J. W., et al. (2001) J Biol. Chem. 276: 4211-4217; Bennet, B. D., et al. (2000) J Biol Chem. 275: 37712-37717), a protease (catalytic) domain and a connecting strand, an integral membrane (transmembrane) domain of about 17 amino acids, and a short cytosolic C-terminal tail of 24 amino acids (Vassar et al., supra). Sequence analyses indicate that BACE belongs to a subfamily of membrane-bound and soluble proteases and contains a classic consensus active site motif found in aspartyl proteases (D T/S G T/S) at positions 93 to 96 and 289 to 292. The entire BACE sequence displays only mild homology with known aspartyl proteases (approximately 30% identity and 37% similarity with members of the mammalian pepsin family), with the highest homology found in the central portion of the extracellular domain.

Accurate information regarding the structure of natural β-secretase is helpful in the design and identification of inhibitors and the enzymatic characterization of the enzyme. A crystal form of a β-secretase, expressed in bacteria, is described in Hong et al., (2000) Science 290:150-153, however this BACE polypeptide is unglycosylated and requires application of extensive refolding methodologies to provide an active enzyme. According to the method of Hong, et al., it was also necessary to form a complex between the polypeptide and an inhibitor

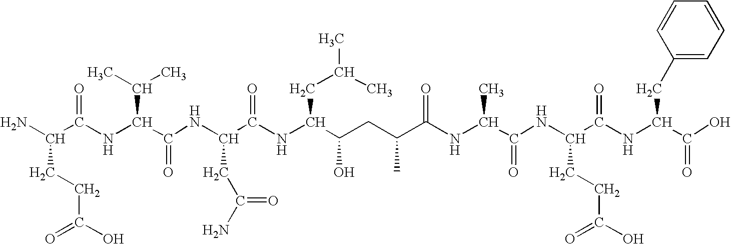

(OM99-2) before crystallization. Typically, refolded polypeptides do not assume the 5 exact three dimensional structure of the native, soluble polypeptide. Thus, there is a need for β-secretase crystals which have similar structure and activity to that of native β-secretase and which can be produced without difficult refolding steps. There is also a need for β-secretase crystals which are uncomplexed and which possess an active site in open configuration to which inhibitors may be easily bound (e.g., by crystal/inhibitor soaking methods).

### SUMMARY OF THE INVENTION

The present invention provides a crystal comprising a polypeptide selected from: (a) a glycosylated, human β-secretase polypeptide characterized by structural coordinates comprising a root mean square deviation of conserved residue backbone atoms of less than about 1.5 Å (e.g., less than about 1.0 Å, less than about 0.5 Å or less than about 0.1 Å) when superimposed on backbone atoms described by structural coordinates of Table 2; (b) a glycosylated, human β-secretase polypeptide complexed with

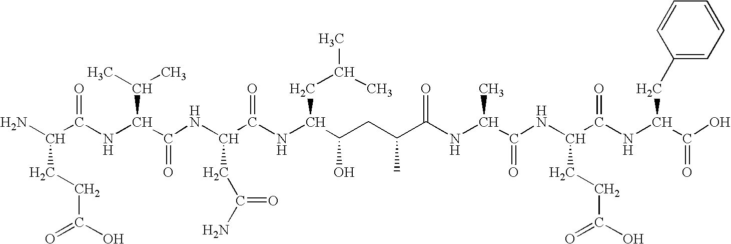

(OM-99-2) characterized by structural coordinates comprising a root mean square deviation of conserved residue backbone atoms of less than about 1.5 Å (e.g., less than about 1.0 Å, less than about 0.5 Å or less than about 0.1 Å) when superimposed on backbone atoms described by structural coordinates of Table 3; and (c) a glycosylated, human β-secretase polypeptide which comprises a pyramidal structure.

Preferably, the crystal comprises a polypeptide selected from a glycosylated, human β-secretase polypeptide comprising the amino acid sequence set forth in SEQ ID NO: 1; a glycosylated, human β-secretase polypeptide comprising the amino acid sequence set forth in SEQ ID NO: 5 complexed with OM-99-2; and a glycosylated, human β-secretase polypeptide comprising the amino acid sequence set forth in SEQ ID NO: 4 which crystal is characterized by a pyramidal structure.

More preferably, the crystal comprises a polypeptide selected from a glycosylated, human β-secretase polypeptide comprising the amino acid sequence set forth in SEQ ID NO: 1 characterized by structural coordinates of Table 2; and a glycosylated, human β-secretase polypeptide comprising the amino acid sequence set forth in SEQ ID NO: 5 complexed with OM-99-2 characterized by structural coordinates of Table 3.

In preferred embodiments, the crystals of the present invention are able to proteolytically cleave a peptide comprising the amino acid sequence KSEVNLDAEFRK (SEQ ID NO: 3). Preferably, the crystal comprises a β-secretase polypeptide comprising an active site in an open configuration. Preferably, the crystal effectively diffracts x-rays for determination of structural coordinates of the polypeptide to a resolution greater than about 5 Å.

Also provided by the present invention is a computer for producing a three-dimensional representation of BACE characterized by the structural coordinates of Table 2 or BACE complexed with OM-99-2 characterized by the structural coordinates of Table 3 or a three-dimensional representation of a homologue of said BACE or said BACE complexed with OM-99-2 wherein the homologue has a root mean square deviation from the backbone atoms of Table 2 or 3 of less than about 1.5 A, preferably less than about 1 Å, more preferably less than about 0.5 Å, and even more preferably, less than about 0.1 Å wherein said computer comprises a machine-readable data storage medium comprising a data storage material encoded with machine-readable data, wherein said data comprises the structure coordinates of Table 2 or 3; a working memory for storing instructions for processing said machine-readable data; a central-processing unit coupled to said working memory and to said machine-readable data storage medium for processing said machine readable data into said three-dimensional representation; and a display unit coupled to said central-processing unit for displaying said three-dimensional representation.

In preferred embodiments of the invention the computer display unit is displaying the three dimensional representation of the polypeptide.

The invention further relates to the three-dimensional structure of BACE and its structure coordinates, e.g., as determined by x-ray crystallography, the use of the structure to solve the structure of BACE homologues, mutants and complexes thereof, and the use of such BACE structures to design inhibitors against BACE.

The invention also provides a method for determining at least a portion of the three-dimensional structure of molecules or molecular complexes which contain at least some structurally similar features to a BACE polypeptide complex.

The present invention also provides methods for obtaining crystals of BACE, preferably, of sufficient quality to determine the three dimensional structure of the polypeptide by x-ray diffraction methods.

Information derived from BACE crystals may be used to model the tertiary structure of related proteins and/or protein complexes. Accordingly, another aspect of the present invention is to provide starting material for the structure determination of other members of the BACE family of proteins. The knowledge of the structure of the BACE family of proteins provides a tool for investigating the mechanism of action of BACE protein. Knowledge of the protein structure allows for the design and synthesis of small molecules which inhibit the functional activities of the BACE protein. One preferred method is structure-based drug design.

Another aspect of this invention is the use of the structure coordinates and atomic details of BACE or mutants or homologues or co-complexes thereof to design, evaluate computationally, synthesize and use inhibitors of BACE that prevent or treat the undesirable physical and pharmacological properties of Alzheimer's disease. These inhibitors may be used in the treatment of Alzheimer's disease.

Still another aspect of the present invention comprises a method for selecting a potential ligand or inhibitor by performing structure-based drug design with a three-dimensional structure determined for the crystal, preferably in conjunction with computer modeling. The potential ligand or inhibitor is then contacted with the BACE polypeptide and the binding thereof is detected. If the ligand is a potential inhibitor of BACE activity, the candidate drug may then be contacted with a cell that expresses BACE and the inhibition of its activity can be measured.

In another embodiment of the invention, a method for obtaining structural information concerning a polypeptide of unknown structure by using the structure coordinates set forth in Table 2 is provided. Such a method comprises the steps of: generating x-ray diffraction data from said crystallized molecule, and applying crystallographic phases derived from at least a portion of the structure coordinates set forth in Table 2 to said x-ray diffraction pattern to generate a three-dimensional electron density map of at least a portion of the molecule.

### DETAILED DESCRIPTION OF THE INVENTION

The present invention includes a crystalline composition including BACE and BACE complexed with OM-99-2. The BACE crystals of the invention, preferably, comprise post-translational modifications (e.g., glycosylation) which are similar to that of native BACE and, furthermore, the crystals are, preferably, formed in the absence of protein refolding steps. Each of these factors enhances the capacity of the crystals of the invention to assume a three-dimensional structure which is similar to that of the native enzyme. The BACE crystals of the invention, preferably, are catalytically active and comprise an active site which is free and available for inhibitor or substrate binding (e.g., by soaking the crystal with the substrate or with the inhibitor). Preferably, the BACE crystals comprise a soluble BACE polypeptide which lacks the transmembrane domain and C-terminal tail of native BACE. The invention also includes novel methods for preparing and for using the crystalline compositions.

In accordance with the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1996) (herein “Ausubel et al., 1996”).

A β-secretase polypeptide or BACE used in this invention is any form of BACE from any species, preferably from an animal, more preferably from a mammal (e.g., mouse, rat, rabbit, dog) and most preferably from a human. Preferably, BACE is a glycosylated protein which is structurally and functionally similar to naturally-occurring human BACE and which has an active site with an open configuration. More preferably, BACE crystals comprise a BACE polypeptide which is a glycosylated, soluble fragment of mature, human BACE which lacks the carboxy-terminal tail and transmembrane domain as well as the amino-terminal propeptide and which includes the amino acid sequence set forth in SEQ ID NO: 1, 4 or 5. The scope of the present invention also includes crystals comprising an immature form of BACE, from which the mature form can be made, which comprises an amino-terminal propeptide. The amino acid sequence of an immature BACE polypeptide (proBACE) is set forth in SEQ ID NO: 2. The scope of the present invention also includes crystals comprising BACE polypeptide as disclosed by Vassar et al., (1999) Science, 286: 735-741-Genbank Accession No. AF190725; Murphy et al., (2001) Neuroreport, 12(3):631-634; Capell et al., (2000) J. Biol. Chem., 275(40):30849-30854 and Haniu et al., (2000) J. Biol. Chem., 275(28):21099-21106. The published sequences also include polypeptides that differ from the BACE protein by having amino acid deletions, substitutions, and additions. BACE used in this invention, preferably, contains catalytic (proteolytic) properties that are comparable to those that have been reported for synthetic peptides derived from the β-amyloid precursor protein (APP) peptide sequence. Examples of APP peptides which may be cleaved by BACE of the present invention are disclosed, for example, in Lin et al., (2000) Proc. Nat. Acad. Sci., 97(4):1456-1460 and Turner et al., (2001) Biochemistry, 40(34):10,001-10,006. The bilobal protein, typically, is lightly glycosylated with glycan attachment accounting for approximately 4 kD of the protein's molecular weight.

Another aspect of the present invention is an uncrystallized mature, soluble fragment of BACE (e.g., SEQ ID NO: 1, 4 or 5) which is the proteolytic cleavage product from a BACE autoprocessing step of, for example, proBACE (e.g., SEQ ID NO: 2) which occurs most efficiently at pH 4.0. When forming the mature, human BACE crystals of the invention, preferably, proBACE is cleaved, to yield mature BACE, by autoprocessing which occurs at the crystallization step.

In addition to BACE polypeptides described in the art, various mutant forms, homologues and variants of BACE can be employed. The terms “mutant” and “mutation” mean any detectable change in genetic material, e.g., DNA, or any process, mechanism, or result of such a change. This includes gene mutations, in which the structure (e.g., DNA sequence) of a gene is altered, any gene or DNA arising from any mutation process, and any expression product (e.g., protein) expressed by a modified gene or DNA sequence. The term “variant” may also be used to indicate a modified or altered gene, DNA sequence, polypeptide or enzyme, etc., i.e., any kind of mutant. Sequence- and function-conservative variants of BACE polypeptides are contemplated for use in the present invention. “Sequence-conservative variants” of BACE are those in which a change of one or more nucleotides in a given codon position results in no alteration in the amino acid encoded at that position. “Function-conservative variants” of BACE are those in which a given amino acid residue in a BACE polypeptide has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like).

Protein or polypeptide homology, or sequence identity, is determined by optimizing residue matches, if necessary, by introducing gaps as required. See, e.g., Needleham, et al. J. Mol. Biol. 48:443-453 (1970); Sankoff et al., “Time Warps, String Edits, and Macromolecules: The Theory and Practice of Sequence Comparison”, Ch. 1, Addison-Wesley, Reading, Mass. (1983); and software packages from IntelliGenetics, Mountain View, Calif. and the University of Wisconsin Genetics Computer Group (GCG), Madison, Wis. This changes when considering conservative substitutions as matches. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. Homologous amino acid sequences are intended to include natural variations of the BACE amino acid sequence. Typical homologous BACE polypeptides used in this invention will have from 50-100% homology (if gaps can be introduced), to 60-100% homology (if conservative substitutions are included), e.g., with BACE comprising the amino acid sequence of SEQ ID NO: 1 or 2. Homology measures are preferably at least about 70%, generally at least 76%, more generally at least 81%, often at least 85%, more often at least 88%, typically at least 90%, more typically at least 92%, usually at least 94%, more usually at least 95%, preferably at least 96%, and more preferably at least 97%, and in particularly preferred embodiments, at least 98% or more. The degree of homology will vary with the length and number of BACE polypeptides compared.

The terms “express” and “expression” mean allowing or causing the information in a gene or DNA sequence to become manifest, e.g., producing a protein by activating the cellular functions involved in transcription and, optionally, translation of a corresponding gene or DNA sequence. A DNA sequence can be expressed using in vitro translation systems (e.g., rabbit reticulocyte lysate-based systems) or in or by a cell to form an “expression product” such as a mRNA or a protein. The expression product, e.g. the resulting protein, may also be referred to as “expressed”.

An insect cell used in this invention is any cell derived from an organism of the class Insecta. Preferably, the insect is Spodoptera fruigiperda (Sf9 or Sf21) or Trichoplusia ni (High 5). Examples of insect expression systems that can be used with the present invention, for example to produce BACE polypeptide, include Bac-To-Bac (Invitrogen Corporation, Carlsbad, Calif.) or Gateway (Invitrogen Corporation, Carlsbad, Calif.).

A BACE polypeptide can be produced by any conventional method, including synthetic methods, such as solid phase, liquid phase and combination solid/liquid phase polypeptide syntheses; recombinant DNA methods, including cDNA cloning, optionally combined with site-directed mutagenesis; and/or purification of the natural products, optionally combined with enzymatic or chemical cleavage methods to produce fragments of naturally-occurring BACE.

It may also be desirable to add amino acids at the amino- or carboxy-terminus of a BACE polypeptide, e.g., to prepare a fusion protein. In one embodiment, the addition is a polyhistidine tag of 5-20 amino acids, preferably 6 amino acids, in length. Preferably, a histidine tag for aiding in purification of a BACE polypeptide is located at the carboxy-terminus. In another embodiment, a myc tag is added to the carboxy-terminus of BACE. The myc tag may be used for detection or immunopurification of BACE. The myc tag and the polyhistidine tag may both be located at the carboxy -terminus or amino-terminus in a doubly-tagged BACE.

The term “enzymatically active” means a protein is catalytically active and, preferably, can hydrolyze a peptide bond of a suitable substrate. Preferably, the term relates to the ability of human BACE to cleave β-amyloid precursor protein or a fragment thereof (e.g., SEQ ID NO: 3); catalytic activity of BACE is discussed above.

The term “active site”, when referring to a BACE polypeptide, describes the area of the polypeptide responsible for peptide recognition and/or peptide bond hydrolysis. For example, the active site for BACE which is used to produce the crystals whose coordinates are set forth in Table 2 includes amino acids Asp72 and Asp268. An active site in an “open configuration” means that the active site is accessible to interaction with a suitable substrate and/or inhibitor. The term “trans-cleavage processing” refers to the ability of one BACE molecule to enzymatically remove the propeptide of another BACE molecule. In general, trans-cleavage processing occurs most efficiently at about pH 4 wherein amino-terminal amino acids are cleaved.

One aspect of the present invention relates to a method of purifying BACE polypeptides and obtaining BACE crystals. Preferably, a BACE polypeptide is produced in a system which produces BACE polypeptide with an active site in open configuration. Preferably, the polypeptide is produced in a system which produces glycosylated BACE; however, the present invention contemplates crystals comprising BACE which has been modified (e.g., post-translationally modified) in any manner which produces an open active site configuration (e.g., phosphorylation, sulfonation, PEGylation). Although BACE may be produced, for example, in mammalian cells (e.g., CHO cells, NIH3T3 cells), it is preferable to produce the protein recombinantly in an insect cell expression system (e.g., an insect cell/baculovirus-based system). Initial purification may be accomplished by nickel chelate chromatography, as previously described in: Ausubel et al. supra. The BACE preparation may be subjected to anion exchange chromatography for further purification. It may also be desirable to subject the BACE preparation to standard size exclusion gel filtration. The protein preparation may be further concentrated using standard techniques. Finally, the preparation is preferably subjected to ultracentrifugation, which produces a monodisperse preparation of BACE. The BACE in the resulting supernatant is useful for crystallization purposes.

A BACE preparation preferably contains a protein stabilizing agent, a salt, a buffering agent and, optionally, a reducing agent or oxygen scavenger. Examples of suitable reducing agents are dithiothreitol (DTT), dithioerythritol (DET) and β-mercaptoethanol (BME). If desired, the reducing agent is present in the buffered solution at a concentration of about 10 mM. Preferably, the reducing agent is BME. The pH of the buffering agent may range from about 4.5 to about 8 (e.g., 5, 6, 7), preferably between about pH 7 and about 8 (e.g., 7.2, 7.4, 7.5, 7.6, 7.8).

Salt concentration appears to be important for the solubility of BACE. Salt may be provided in a concentration of more than about 300 mM (e.g., 500 mM). Various salts are routinely used in the art in similar methods, including sodium chloride and imidazole.

A “precipitant” is a compound that decreases the solubility of a polypeptide in a concentrated solution. Alternatively, the term “precipitant” can be used to refer to a change in physical or chemical parameters which decreases polypeptide solubility, including temperature, pH and salt concentrations. Precipitants induce crystallization by forming an energetically unfavorable precipitant-depleted layer around the polypeptide molecules. To minimize the relative amount of this depletion layer, the polypeptides form associations and, ultimately, crystals. This process is explained in Weber, Advances in Protein Chemistry 41:1-36 (1991) which is incorporated by reference. In addition to precipitants, other materials are sometimes added to the polypeptide crystallization solution. These include buffers, such as Hepes, to adjust the pH of the solution (and hence surface charge on the peptide) and salts, such as sodium chloride, lithium chloride and sodium citrate, to reduce the solubility of the polypeptide. Various precipitants are known in the art and include the following: ammonium sulfate, ethanol, 3-ethyl-2,4 pentanediol; and many of the polyglycols, such as polyethylene glycol. A suitable precipitant for crystallization of BACE polypeptide complex is polyethylene glycol (PEG), preferably PEG with a molecular weight of 6000 Da, which combines some of the characteristics of the salts and other organic precipitants.

“Monodisperse” and “predominantly uniform molecular species”, in reference to BACE, are used interchangeably to indicate that the mean radius of particles comprising the BACE varies by less than about 30%, preferably less than about 15%, as determined by, e.g., conventional dynamic light scattering methods. A monodisperse BACE in solution preferably exists in a monomeric form, however, oligomers (e.g., dimers, trimers, tetramers, etc.) may also exist. Such mixtures of BACE have subunits of a molecular weight of about 55 kDa.

Crystallization may be accomplished by using any of the known methods in the art (Giege, et al., (1994) Acta Crystallogr. D50: 339-350; McPherson, (1990) Eur. J. Biochem. 189: 1-23). Such techniques include microbatch, hanging drop, seeding and dialysis. Preferably, hanging-drop vapor diffusion (McPherson, (1976) J. Biol. Chem. 251: 6300 -6303) or microbatch methods (Chayen (1997) Structure 5: 1269-1274) are used. In each of these methods, it is important to promote continued crystal growth after nucleation by maintaining a supersaturated solution. In the microbatch method, polypeptide is mixed with precipitants to achieve supersaturation, the vessel is sealed and set aside until crystals appear. In the dialysis method, polypeptide is retained in a sealed dialysis membrane which is placed into a solution containing precipitant. Equilibration across the membrane increases the precipitant concentration thereby causing the polypeptide to reach supersaturation levels. It is desirable to use a BACE protein preparation having a concentration of at least about 1 mg/mL and preferably about 10 mg/mL to about 20 mg/mL. Crystallization may be best achieved in a precipitant solution containing polyethylene glycol 1000-20,000 (PEG; average molecular weight ranging from about 1000 to about 20,000 Da), preferably about 5000 to about 7000 Da, more preferably about 6000 Da, with concentrations ranging from about 10% to about 30% (w/v). It may also be desirable to include a protein stabilizing agent. If glycerol is chosen as the protein stabilizing agent, it is preferably provided at a concentration ranging from about 0.5% to about 20%. A suitable salt, such as sodium chloride, lithium chloride or sodium citrate may also be desirable in the precipitant solution, preferably in a concentration ranging from about 1 mM to about 1000 mM. The precipitant is preferably buffered to a pH of from about 3.0 to about 5.0, preferably about 4.0. Specific buffers useful in the precipitant solution may vary and are well-known in the art (Scopes, Protein Purification: Principles and Practice, Third ed., (1994) Springer-Verlag, New York). Examples of useful buffers include, but are not limited to, Hepes, Tris, MES and acetate. Crystals routinely grow at a wide range of temperatures. It is, however, preferred that crystals form at temperatures between about 2° C. and about 26° C., and more preferably at about 2° C. to about 8° C., most preferably at about 4° C.

The crystals of the present invention have a wide range of uses. For example, high quality crystals are suitable for x-ray or neutron diffraction analysis to determine the three dimensional structure of BACE and in particular to assist in the identification of the protein's active and effector sites. Knowledge of these sites and solvent accessible residues allow structure-based design and construction of agonists and antagonists for BACE.

In addition, crystallization itself can be used as a purification method. In some instances, a polypeptide or protein crystallizes from a heterogeneous mixture into crystals. Isolation of such crystals by filtration and/or centrifugation, followed by redissolving the polypeptide affords a purified solution suitable for use in growing high-quality crystals which are preferred for diffraction analysis.

Once a crystal of the present invention is grown, x-ray diffraction data can be collected. One method for determining structure with x-ray diffraction data includes use of synchrotron radiation, under standard cryogenic condition; however, alternative methods may also be used. For example, crystals can be characterized by using x-rays produced by a conventional source, such as a sealed tube or a rotating anode. Methods of characterization include, but are not limited to, precession photography, oscillation photography and diffractometer data collection.

The crystallizable compositions provided by this invention may be amenable to x- ray crystallography for providing the three-dimensional structure of a BACE polypeptide. The present invention includes crystals which effectively diffract x-rays for the determination of the atomic coordinates of BACE to a resolution of greater than about 5.0 Ångströms (e.g., about 4.5 Å, about 4.0 Å, about 3 Å, about 2.5 Å, about 2 Å, about 1 Å, about 0.5 Å, about 0.1 Å), preferably greater than about 4.0 Ångströms (e.g., about 3 Å, about 2.5 Å, about 2 Å, about 1 Å, about 0.5 Å, about 0.1 Å), more preferably greater than about 2.8 Ångströms (e.g., about 2.5 Å, about 2 Å, about 1 Å, about 0.5 Å, about 0.1 Å) and most preferably greater than about 2.0 Ångströms (e.g., about 1.5 Å, about 1.0 Å, about 0.5 Å, about 0.1 Å).

The present invention includes BACE crystals whose three-dimensional structure is described by the structure coordinates set forth in Table 2 or 3. The scope of the present invention also includes crystals which possess structural coordinates which are similar to those set forth in Table 2 or 3; preferably, the crystals or the soluble polypeptides which are used to form the crystals exhibit BACE catalytic activity (see above) and, preferably, the crystals include glycosylated BACE. Most preferably, the crystals include a polypeptide which includes the amino acid sequence of SEQ ID NO: 1 or 5. Structural similarity between crystals is discussed in detail below.

The term “structure coordinates” refers to Cartesian coordinates derived from mathematical equations related to the patterns obtained on diffraction of a beam of x-rays by the atoms (scattering centers) of a molecule. The diffraction data are used to calculate electron density maps and to establish the positions of the individual atoms of the molecule.

Those of skill in the art will understand that a set of structure coordinates for an enzyme or an enzyme-complex or a portion thereof, is a relative set of points that define a shape in three dimensions. Thus, it is possible that an entirely different set of coordinates could define a similar or identical shape. Moreover, slight variations in the individual coordinates will have little effect on overall shape.

The present invention includes crystals exhibiting structural coordinates which are similar to those set forth in Table 2 or 3 but for crystallographic permutations of the structure coordinates, fractionalization of the structure coordinates, additions, subtractions, rotations or translations to sets of the structure coordinates or any combinations of the above.

Alternatively, modifications in the crystal structure due to mutations, additions, substitutions, and/or deletions of amino acids, or other changes in any of the components that make up the crystal may also account for variations in structure coordinates. If such variations are within an acceptable standard error as compared to the coordinates of Table 2 or 3, the resulting three-dimensional shape is considered to be the same and, accordingly, the modified crystal is considered to be within the scope of the present invention.

Various computational analyses may be necessary to determine whether a crystal is sufficiently similar to the crystals whose structural coordinates are set forth in Table 2 or 3 as to be considered the same. Such analyses may be carried out in current software applications, such as the Molecular Similarity application of QUANTA (Molecular Simulations Inc., San Diego, Calif.) version 4. 1, and as described in the accompanying User's Guide.

The Molecular Similarity application permits comparisons between different structures, different conformations of the same structure, and different parts of the same structure. In general, the procedure used in Molecular Similarity to compare structures is divided into four steps: 1) input the structures to be compared; 2) define the atom equivalences in these structures; 3) perform a fitting operation; and 4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention we will define equivalent atoms as protein backbone atoms (N, Cα, C and O) for all conserved residues between the two structures being compared.

When a rigid fitting method is used, the working structure is translated and rotated to obtain an optimum fit with the target structure. The fitting operation uses a least squares fitting algorithm that computes the optimum translation and rotation to be applied to the moving structure, such that the root mean square difference of the fit over the specified pairs of equivalent atom is an absolute minimum. This number, given in Angstroms, is reported by QUANTA.

The term “root mean square deviation” (RMSD) is a commonly known term in the art which, in general, means the square root of the arithmetic mean of the squares of the deviations from the mean distance of corresponding atoms. It is a way to express the deviation or variation from a trend or object.

For the purpose of this invention, any set of structure coordinates of a molecule that has a RMSD of conserved residue backbone atoms (N, Cα, C, O) of less than about 1.5 Å when superimposed—using backbone atoms—on the relevant structure coordinates of Table 2 or 3 are considered identical and are within the scope of the present invention. Preferably the crystal is a catalytically active human, glycosylated BACE crystal (e.g., SEQ ID NO: 1 or 5). Preferably, the root mean square deviation is less than about 1.0 Å, even more preferably, the root mean square deviation is less than about 0.5 Å and most preferably, the root mean square deviation is less than about 0.1 Å.

The term “least squares” refers to a method based on the principle that the best estimate of a value is that in which the sum of the squares of the deviations of observed values is a minimum.

In accordance with the present invention, the structure coordinates of the BACE polypeptide and portions thereof may be stored in a machine-readable storage medium. Such data may be used for a variety of purposes, such as drug discovery and x-ray crystallographic analysis of a protein crystal (e.g., for producing a three-dimensional representation of BACE). Accordingly, one aspect of this invention provides a machine-readable data storage medium comprising a data storage material encoded with the structure coordinates set forth in Table 2 or 3. The machine-readable data storage medium may also include any set of structure coordinates of a molecule that has a root mean square deviation of conserved residue backbone atoms (N, Cα, C, O) of less than about 1.5 Å, preferably, less than about 1.0 Å, more preferably less than about 0.5 Å and even more preferably less than about 0.1 Å when superimposed—using backbone atoms—on the relevant structure coordinates of Table 2 or 3.

A computer system, useful in reading the machine readable data storage medium, includes a computer comprising a central processing unit (“CPU”) and a memory storage device and is also within the scope of the present invention. In general, the computer system may be any computer with an operating system such as MS-DOS, PC-DOS, Windows, OS/2, Unix, Unix variant or MacOS. Particularly preferred computer systems are the Silicon Graphics Octane workstation or Compaq AlphaServer DS20. Other hardware systems and software packages will be known to those skilled in the art.

Input hardware coupled to the computer system by input line, may be implemented in a variety of ways. Machine-readable data of this invention may be input via the use of a modem or modems connected by a telephone line or a dedicated data line. Alternatively or additionally, the input hardware may comprise CD-ROM drives or disk drives. A keyboard may also be used as an input device.

Output hardware, coupled to the computer system by output lines, may similarly be implemented by conventional devices. By way of example, output hardware may include a display terminal (e.g., a cathode ray tube (CRT)) for displaying a graphical representation of the three dimensional structure of BACE or a portion thereof using a program such as INSIGHT (Molecular Simulations Inc., San Diego, Calif.) or QUANTA as described herein. Output hardware might also include a printer, so that hard copy output may be produced, or a disk drive, to store system output for later use. In preferred embodiments, the computer possesses a display which is displaying a three dimensional representation of BACE or a fragment or homologue thereof.

In operation, the central processing unit (CPU) coordinates the use of the various input and output devices, coordinates data accesses from mass storage and accesses to and from working memory, and determines the sequence of data processing steps. A number of programs may be used to process the machine-readable data of this invention. Such programs are discussed in reference to the computational methods of drug discovery as described herein. Specific references to components of the computer system are included as appropriate throughout the following description of the data storage medium.

A magnetic data storage medium can be encoded with a machine-readable data by a computer system as described above. Storage medium may be, for example, a conventional floppy diskette or hard disk, having a suitable substrate, which may be conventional, and a suitable coating, which may be conventional, on one or both sides, containing magnetic domains whose polarity or orientation can be altered magnetically. The magnetic domains of the coating of medium may be polarized or oriented so as to encode, in a manner which may be conventional, machine readable data, such as that described herein, for execution by a system as described herein. Storage medium may also have an opening for receiving the spindle of a disk drive or other data storage device. Alternatively, an optically-readable data storage medium can be encoded with such machine-readable data, or a set of instructions. Medium can be a conventional compact disk read only memory (CD-ROM) or a rewritable medium such as a magneto-optical disk which is optically readable and magneto-optically writable.

In general, in the case of CD-ROM, as is well known, disk coating is reflective and is impressed with a plurality of pits to encode the machine-readable data. The arrangement of the pits is read by reflecting laser light off the surface of the coating. A protective coating, which preferably is substantially transparent, is provided on top of the coating.

In general, in the case of a magneto-optical disk, as is well known, disk coating has no pits, but has a plurality of magnetic domains whose polarity or orientation can be changed magnetically when heated above a certain temperature, as by a laser. The orientation of the domains can be read by measuring the polarization of laser light reflected from the coating. The arrangement of the domains encodes the data as described above.

The present invention permits the use of structure-based drug design techniques to design, select, and synthesize chemical entities, including inhibitory compounds that are capable of binding to a BACE polypeptide. Also, de novo and iterative drug design methods can be used to develop drugs from the structure of the BACE crystals of this invention.

One particularly useful drug design technique enabled by this invention is structure-based drug design. Structure-based drug design is a method for optimizing associations between a protein and a compound by determining and evaluating the three-dimensional structures of successive sets of protein/compound complexes.

Those skilled in the art will appreciate that association of natural ligands or substrates with the binding pockets of their corresponding receptors or enzymes is the basis of many biological mechanisms of action. The term “binding pocket”, as used herein, may refer to any region of a molecule or molecular complex, that, as a result of its shape, favorably associates with another chemical entity or compound. Similarly, drugs may exert their biological effects through association with the binding pockets of receptors and enzymes. Such association may occur with all or any part of the binding pockets. An understanding of such associations will help lead to the design of drugs having more favorable associations with the target enzyme, and thus, improved biological effects. Therefore, this information is valuable in designing potential enzyme inhibitors, such as inhibitors of BACE.

In iterative structure-based drug design, crystals of a series of protein/compound complexes are obtained and then the three-dimensional structure of each complex is solved. Such an approach provides insight into the association between the proteins and compounds of each complex. This is accomplished by selecting compounds with inhibitory activity, obtaining crystals of a new polypeptide, solving the three-dimensional structure of the polypeptide, and comparing the associations between the new protein and previously solved protein. By observing how changes in the compound affected the protein/compound associations, these associations may be optimized.

In some cases, iterative structure-based drug design is carried out by forming successive protein-compound complexes and then crystallizing each new complex. Alternatively, a pre-formed protein crystal is soaked in the presence of an inhibitor, thereby forming a protein/compound complex and obviating the need to crystallize each individual protein/compound complex. Advantageously, BACE crystals provided by this invention may be soaked in the presence of a compound or compounds, such as BACE inhibitors, substrates or other ligands to provide novel BACE/compound crystal complexes. As used herein, the term “soaked” may refer to a process in which the crystal is transferred to a solution containing the compound of interest.

The structure coordinates set forth in Table 2 or 3 can also be used to aid in obtaining structural information about another crystallized molecule or molecular complex. This may be achieved by any of a number of well-known techniques, including molecular replacement.

The structure coordinates set forth in Table 2 or 3 can also be used for determining at least a portion of the three-dimensional structure of molecules or molecular complexes which contain at least some structurally similar features to BACE. In particular, structural information about another crystallized molecule or molecular complex may be obtained by well-known techniques, including molecular replacement.

Therefore, another aspect of this invention provides a method of utilizing molecular replacement to obtain structural information about a crystallized molecule or molecular complex, whose structure is unknown, comprising the steps of generating an x-ray diffraction pattern from said crystallized molecule or molecular complex and applying crystallographic phases derived from at least a portion of the structure coordinates set forth in Table 2 or 3 to the x-ray diffraction pattern to generate a three-dimensional electron density map of the molecule or molecular complex whose structure is unknown.

Once the structure coordinates of a protein crystal have been determined, they are useful in solving the structures of other crystals. In addition, the structure of BACE homologues may be determined from the structural coordinates of the present invention. For example, polypeptides may be crystallized and their structure elucidated by, for example, difference Fourier techniques and molecular replacement.

By using molecular replacement, all or part of the structure coordinates of the BACE polypeptide provided by this invention (and set forth in Table 2 or 3) can be used to determine the previously unknown structure of a crystallized molecule or molecular complex more quickly and efficiently than attempting to determine such information ab initio.

Molecular replacement provides an accurate estimation of the phases for an unknown structure. Phases are a factor in equations used to solve crystal structures that cannot be measured experimentally. Obtaining accurate values for the phases, by methods other than molecular replacement, is a time-consuming process. However, when the crystal structure of a protein containing a homologous portion has been solved, the phases from the known structure may provide a satisfactory estimate of the phases for the unknown structure.

Thus, this method involves generating a preliminary model of a molecule or molecular complex whose structure coordinates are unknown, by orienting and positioning the relevant portion of the BACE crystal according to Table 2 or 3 within the unit cell of the crystal of the unknown molecule or molecular complex so as best to account for the observed x-ray diffraction pattern amplitudes to generate an election density map of the structure whose coordinates are unknown. This, in turn, can be subjected to any well-known model building and structure refinement techniques to provide a final, accurate structure of the unknown crystallized molecule or molecular complex (Lattman, “Use of the Rotation and Translation Functions”, in Meth. Enzymol., 115: 55-77 (1985); Rossman, ed., “The Molecular Replacement Method”, Int. Sci. Rev. Ser., No. 13, Gordon & Breach, New York (1972)).

Phase information from the structure coordinates of the present invention may be used to elucidate the structure of other crystals. For example, the structure of BACE in complex with other atoms or molecules may be elucidated. Such complexes include, for example, those containing atoms soaked into or cocrystallized within the crystal lattice. Other structures which can be elucidated using the phase information of the present invention include for example other proteases or homologues or mutants thereof having sufficient three-dimensional structure similarity to BACE complex as to be solved using molecular replacement. Examples of such proteins include, but are not limited to, cathepsin D, renin and pepsin. Also, these protein molecules in a complex with a small molecule substrate(s), inhibitor(s), transition state analog(s), product(s) or analog(s) of any of these may also be solved using the phase information of the present invention. Other complexes whose structure can be elucidated from the phase information of the present invention include a BACE complexed with an inhibitor. Complexes containing a combination of the above molecules may also be solved using the phase information of the present invention.

The structure of any portion of any crystallized molecule or molecular complex that is sufficiently homologous to any portion of the BACE protein can be solved by this method. The difference Fourier method simply calculates an electron density map using phases calculated from the structure coordinates and observed diffraction amplitudes from a crystal of an unknown structure. This method is often used to solve structures of protein/ligand complexes where the ligand is small and does not affect the crystal form significantly.

In a preferred embodiment, the method of molecular replacement is utilized to obtain structural information about a molecule wherein the molecule comprises a BACE polypeptide complex. The structure coordinates of BACE provided by this invention are particularly useful in solving the structure of other crystal forms of BACE polypeptide complexes. This approach enables the determination of the optimal sites for interaction between chemical entities, including interaction of candidate inhibitors with BACE.

BACE crystals may be studied using well-known x-ray diffraction techniques and may be refined versus x-ray data to 3 Å resolution or better to an Rfree value of about 0.40 or less using computer software such as X-PLOR (Yale University, 1992, distributed by Molecular Simulations, Inc.; see e.g., Blundell & Johnson, supra; Meth, Enzymol., vol. 114 & 115, H. W. Wyckoff et al., eds., Academic Press (1985)). This information may be used to optimize known BACE inhibitors and to design new BACE inhibitors.

### EXAMPLES

The following examples are provided to describe the present invention in greater detail and should not be construed to limit the scope of the invention.

The following Examples describe methods for producing recombinant β-secretase crystals suitable for structure based drug design. Enzymatically active, human β-secretase was produced in insect cells by recombinant means, using recombinant vectors and baculoviruses containing cDNA encoding the 55KDa β-secretase protein. The β-secretase constructs incorporated a Myc-tag and a six-histidine tag at the C-terminus. Expressed β-secretase was purified to homogeneity using a combination of purification steps, including anion exchange chromatography, nickel chelate chromatography and gel filtration chromatography. The resulting monodisperse, enzymatically active β-secretase preparation was suitable for crystallization. X-ray diffraction quality crystals were grown using a hanging-drop vapor diffusion method. The β-secretase solution (1 μl; 10-20 mg/ml protein) in 20 mM Hepes, pH 7.5, 150 mM NaCl was mixed with an equal volume of precipitant, placed on the underside of a siliconized glass coverslip, and sealed in close proximity to 1 ml of the precipitant solution. The precipitant solution contained polyethyleneglycol 6000 with concentrations ranging from 6 to 30% (w/v). The precipitant solution also contained 200 mM to 1000 mM lithium chloride in a 0.1 M sodium citrate buffer between pH 3.8 and 4.6. After incubation at 4° C for 10 to 30 days, small rectangular rod crystals formed. Crystals grew to terminal size within one month with dimensions up to 0.03×0.03×0.2 mm. After transfer of the crystals to cryoprotectant which contains 20% glycerol higher than the crystallization medium, the crystals can be frozen directly in liquid propane for storage prior to diffraction data collection or frozen in a gaseous nitrogen stream immediately before diffraction data collection.

### Example 1

Cloning of Human β-Secretase

Human β-secretase cDNA clone with C-terminal Myc-tag and 6×His-tag was inserted into a pcDNA4 vector and two PCR primers, sBACE1mutF(5′-ctcgagtctagagggcccttcgaacaaaaactc-3′) and sBACE1mutR (5′-ggttgactcatctgtctgtggaatgttgtagcc-3'), were used to delete the transmembrane domain and C-terminal tail of β-secretase. The sBACE/yc/6×His (myc-tag and 6-histidine-tag at the C-terminus) insert was then excised from the pcDNA4 vector using restriction enzymes HindIII and PmeI. The insert was blunt ended with Klenow enzyme and subcloned into the Stul site of pFASTBACI (A) vector provided in Bac-to-Bac Baculovirus Expression System (GIBCO/BRL, Rockville, Md., USA). The amino acid sequence of the polypeptide encoded by the insert is set forth in SEQ ID NO: 2.

### Example 2

Production of Recombinant Baculoviruses

Recombinant baculovirus was produced as described in the Bac-to-Bac expression manual (Gibco BRL, Rockville, Md., USA; SF900-II) following the protocol for transposition, isolation of recombinant bacmid DNA, transfection of Sf9 cells with recombinant bacmid DNA and harvesting/storage of recombinant baculovirus. Recombinant virus was then plaque purified according to the manual and amplified by the infection of suspension cultures using a multiplicity of infection of 0.05.

### Example 3

Expression and Recovery of Baculovirus Recombinant Pro-β-Secretase (proBACE).

Spodoptera frugiperda (Sf9 and Sf21) and Tridchoplusia ni (High Five™; Invitrogen, Carlsbad, Calif., USA) cells were grown in suspension at 27° C. in serum free media (SF900-II; Gibco BRL, Rockville, Md,. USA). Multiplicity of infection (MOI), cell type and time course of expression were all studied to obtain optimal protein expression yields of secreted soluble β-secretase. Sf9 cells infected with a MOI of 5 and incubated for 72 hours were determined to be optimal for protein secretion into the growth media and resulted in expression yields of approximately 4 mg/L.

### Example 4:

Purification of SF-9 derived pro-o-Secretase (proBACE)

Thirty shake flasks (1.0 liters/flask) of conditioned media Sf-9 cells were collected by centrifugation at 1000 g for 15 minutes. The combined supernatant was concentrated using a Pellicon Laboratory System (Millipore Corporation, Bedford, Mass., USA) with four 30K molecular weight cutoff membrane cassettes; P2C030C05 Pellicon 2- cassette) to 4 L. An equal volume of 50 mM Hepes, pH 8.0 was added to the retentate, which was concentrated to 4 L. This procedure was repeated 4 times. The final retentate (pH 8.0, conductivity=2.8 mS/cm) was applied to a 400 ml Q-sepharose Fast Flow anion exchange column (Millipore Corporation, Bedford, Mass.; XK 50/30) pre-equilibrated with Buffer A (20 mM Hepes, pH 8.0) at 50 ml /min. The column was washed with 10 column volumes (CV) of Buffer A, and the protein was eluted with a sodium chloride gradient (0-300 mM). Sodium chloride and imidazole (Sigma Chemical Company, St. Louis, Mo.) were added to the combined eluate fractions at final concentrations of 500 mM and 20 mM, respectively. The resulting solution was applied to a 30 ml Ni-NTA Superflow (Qiagen Corporation, Valencia, Calif.) column (Millipore Corporation; XK 26/30) at 5 ml/min. The column was washed with 10 CV of Buffer B (20 mM Hepes, 20 mM imidazole, 500 mM sodium chloride) and the protein was eluted with 3 CV of Buffer C (20 mM Hepes, pH 8.0, 250 mM imidazole, 500 mM sodium chloride). The eluate fractions containing proBACE were pooled and concentrated to 2 ml, and injected to a Superdex 200 gel filtration column (Millipore Corporation; HighLoad, 26/60). Buffer D (20 mM Hepes, pH 7.5, 150 mM sodium chloride) was applied to the column at 4 ml/min. Fractions containing proBACE were combined based on SDS-PAGE analysis. The pooled fractions were concentrated to 20 mg/ml. The highly purified β-secretase was monodisperse.

TABLE 1
The recovery of proBACE in the purification steps

| Step | Volume | Total protein | | Recovery |
|---|---|---|---|---|
| Conditioned media | 285 L | | | |
| Pellicon concentrate | 8.5 L | 5.1 | g | |
| Q-sepharose (400 | | | | |
| ml) | | | | |
| load | 8.5 L | 5.1 | g | |
| Ft/wash | 12 L | 2.5 | g | |
| eluant | 2.5 L | 1.9 | g | |
| Ni-NTA (30 ml) | | | | |
| load | 2.5 L | 1.9 | g | |
| eluant | 0.27 L | 33 | mg | |
| Superdex 200 | | | | |
| load | 2.0 ml | 33 | mg | |
| eluant | 25 ml | 26 | mg | 0.9 mg/L media |

### Example 5

Enzymatic Activity of SF-9 Derived β-Secretase

To assess the functionality of β-secretase purified from Sf-9 cells, an HPLC assay was developed using a peptide substrate derived from the sequence of “Swedish” amyloid protein peptide (Dreyer et al., (1994) Eur J. Biochem 224: 265-271). In these assays, ProBACE (SEQ ID NO: 2) and mature BACE (SEQ ID NO: 1) were tested. The substrate, Biotin-KSEVNL*DAEFRK-Fluorescein (SEQ ID NO: 3) (* indicates cleavage site), was determined to be a suitable substrate for β-secretase, having a specificity constant (k_{cat}/Kₘ) of 1500 ±100 M⁻¹s⁻¹. The activity of S59 derived ,I-secretase with this substrate sequence is consistent with β-secretase derived from other expression systems (see e.g., Lin et. al., (2000) PNAS USA 97:1456), which confirms that 59-derived β-secretase is enzymatically active. The enzymatic activity of the proBACE and the mature BACE, with this substrate, were determined to be equivalent.

### Example 6

Crystallization of SF-9 derived β-Secretase

ProBACE (SEQ ID NO: 2) in 20 mM Hepes, pH 7.5, 150 mM NaCl was concentrated by centrifugal filtration to 0.18 to 0.36 mM (10-20 mg / ml) followed by ultra-centrifugation prior to crystallization. Vapor diffusion crystallization experiments were conducted using the hanging drop method. Crystals were grown from a droplet containing 1 μl of protein and 1 μl of the reservoir solution which contained 0.1 M sodium citrate (Fluka BioChemika, Germany), pH 4.0, 10-30 % polyethylene glycol 6000 (Fluka BioChemika, Germany), and 0.2-1.0 M lithium chloride (Fluka BioChemika, Germany). At pH 4.0, the proBACE was autoprocessed, within the droplet, to yield mature BACE (SEQ ID NO: 1). During the autoprocessing step, the carboxy -terminal myc and His tags are cleaved from the polypeptide. Crystallization plates were incubated at 4° C., which grew rectangular rods (0.02×0.2 mm) over 10-30 days.

| | |
|---|---|
| Mature BACE | |
| ETDEEPEEPG RRGSFVEMVD NLRGKSGQGY YVEMTVGSPP QTLNILVDTG | (SEQ ID NO: 1) |
| SSNFAVGAAP HPFLHRYYQR QLSSTYRDLR KGVYVPYTQG KWEGELGTDL | |
| VSIPHGPNVT VRANIAAITE SDKFFINGSN WEGILGLAYA EIARPDDSLE | |
| PFFDSLVKQT HVPNLFSLQL CGAGFPLNQS EVLASVGGSM IIGGIDHSLY | |
| TGSLWYTPIR REWYYEVIIV RVEINGQDLK MDCKEYNYDK SIVDSGTTNL | |
| RLPKKVFEAA VKSIKAASST EKFPDGFWLG EQLVCWQAGT TPWNIFPVIS | |
| LYLMGEVTNQ SFRITILPQQ YLRPVEDVAT SQDDCYKFAI SQSSTGTVMG | |
| AVIMEGFYVV FDRARKRIGF AVSACHVHDE FRTAAVEGPF VTLDMEDCGY | |
| NIPQTDESTL E | |
| ProBACE | |
| TQHGIRLPLR SGLGGAPLGL RLPRETDEEP EEPGRRGSFV EMVDNLRGKS | (SEQ ID NO: 2) |
| GQGYYVEMTV GSPPQTLNIL VDTGSSNFAV GAAPHPFLHR YYQRQLSSTY | |
| RDLRKGVYVP YTQGKWEGEL GTDLVSIPHG PNVTVRANIA AITESDKFFI | |
| NGSNWEGILG LAYAEIARPD DSLEPFFDSL VKQTHVPNLF SLQLCGAGFP | |
| LNQSEVLASV GGSMIIGGID HSLYTGSLWY TPIRREWYYE VIIVRVEING | |
| QDLKMDCKEY NYDKSIVDSG TTNLRLPKKV FEAAVKSIKA ASSTEKFPDG | |
| FWLGEQLVCW QAGTTPWNIF PVISLYLMGE VTNQSFRITI LPQQYLRPVE | |
| DVATSQDDCY KFAISQSSTG TVMGAVIMEG FYVVFDRARK RIGFAVSACH | |
| VHDEFRTAAV EGPFVTLDME DCGYNIPQTD ESTLE | |

### Example 7

Crystallographic Analysis of β-Secretase and Model Building and Refinement

Crystals were removed from the crystallization droplet by adding 20% glycerol, which permitted freezing under both cold nitrogen stream and liquid propane. Diffraction data of the β-secretase crystal was determined from a Rigaku R-Axis IV image plate detector mounted on a Rigaku RU-HR rotating anode generator Cu radiation 1.54 Å operating at 100 mA and 50 kV.

Data Collection Statistics:

| | |
|---|---|
| Resolution | 40-3.4 Å |
| No. of collected reflections | 103348 |
| No. of unique reflections (F >= 0) | 18402 |
| R-sym | 0.082 |
| Percent of theoretical (I/s >= 1) | 99% |
| Unit Cell | a = 74 Å, b = 130 Å, |
| | c = 134 Å, α = β = γ = 90° |
| Space Group | P2₁2₁2₁ |
| Asymmetric unit | 2 molecules |

The underlying structure of the β-secretase crystals was solved using molecular replacement as implemented in CNX (MSI Inc.). The molecular replacement protocol as described in the CNX manual was followed with minor modifications. The search model consisted of molecule A from the β-secretase structure deposited in the PDB (pdb code 1FKN). Analysis of the molecular replacement solution shows two molecules in the asymmetric unit. The active site of both molecules is open and not blocked by crystal contacts.

TABLE 2
Structural coordinates for BACE.

| Res. | Atom | # | X | Y | Z | B | C |
|---|---|---|---|---|---|---|---|
| GLU | CB | 41 | −8.1 | −77.9 | 86.1 | 17 | A |
| GLU | CG | 41 | −8.7 | −79.2 | 85.5 | 26 | A |
| GLU | CD | 41 | −10.0 | −79.6 | 86.3 | 32 | A |
| GLU | OE1 | 41 | −10.9 | −78.8 | 86.6 | 29 | A |
| GLU | OE2 | 41 | −10.2 | −80.9 | 86.5 | 32 | A |
| GLU | C | 41 | −7.5 | −77.3 | 83.8 | 9 | A |
| GLU | O | 41 | −7.1 | −78.2 | 83.0 | 7 | A |
| GLU | N | 41 | −6.8 | −75.9 | 85.8 | 1 | A |
| GLU | CA | 41 | −7.0 | −77.3 | 85.3 | 10 | A |
| MET | N | 42 | −8.3 | −76.3 | 83.4 | 10 | A |
| MET | CA | 42 | −8.8 | −76.2 | 82.0 | 7 | A |
| MET | CB | 42 | −10.3 | −75.9 | 82.0 | 6 | A |
| MET | CG | 42 | −11.2 | −77.1 | 82.2 | 4 | A |
| MET | SD | 42 | −12.9 | −76.8 | 82.3 | 1 | A |
| MET | CE | 42 | −13.2 | −76.7 | 84.0 | 1 | A |
| MET | C | 42 | −8.0 | −75.1 | 81.3 | 6 | A |
| MET | O | 42 | −7.9 | −75.0 | 80.1 | 6 | A |
| VAL | N | 43 | −7.6 | −74.1 | 82.2 | 2 | A |
| VAL | CA | 43 | −6.8 | −73.0 | 81.7 | 2 | A |
| VAL | CB | 43 | −6.0 | −72.5 | 82.9 | 1 | A |
| VAL | CG1 | 43 | −5.1 | −71.3 | 82.4 | 1 | A |
| VAL | CG2 | 43 | −7.0 | −72.0 | 83.9 | 1 | A |
| VAL | C | 43 | −5.8 | −73.4 | 80.6 | 2 | A |
| VAL | O | 43 | −5.2 | −74.5 | 80.7 | 5 | A |
| ASP | N | 44 | −5.5 | −72.5 | 79.7 | 4 | A |
| ASP | CA | 44 | −4.6 | −72.8 | 78.6 | 6 | A |
| ASP | CB | 44 | −3.2 | −73.1 | 79.2 | 13 | A |
| ASP | CG | 44 | −2.3 | −71.9 | 79.2 | 22 | A |
| ASP | OD1 | 44 | −2.4 | −71.1 | 80.1 | 28 | A |
| ASP | OD2 | 44 | −1.5 | −71.8 | 78.2 | 24 | A |
| ASP | C | 44 | −5.0 | −73.9 | 77.7 | 7 | A |
| ASP | O | 44 | −4.2 | −74.4 | 77.0 | 14 | A |
| ASN | N | 45 | −6.3 | −74.4 | 77.8 | 5 | A |
| ASN | CA | 45 | −6.6 | −75.5 | 76.9 | 7 | A |
| ASN | CB | 45 | −7.8 | −76.3 | 77.5 | 5 | A |
| ASN | CG | 45 | −9.1 | −75.5 | 77.6 | 1 | A |
| ASN | OD1 | 45 | −9.1 | −74.3 | 77.4 | 1 | A |
| ASN | ND2 | 45 | −10.2 | −76.1 | 77.8 | 1 | A |
| ASN | C | 45 | −6.9 | −75.2 | 75.5 | 7 | A |
| ASN | O | 45 | −7.1 | −76.1 | 74.6 | 7 | A |
| LEU | N | 46 | −6.9 | −73.9 | 75.1 | 4 | A |
| LEU | CA | 46 | −7.1 | −73.4 | 73.7 | 3 | A |
| LEU | CB | 46 | −8.0 | −72.2 | 73.7 | 3 | A |
| LEU | CG | 46 | −9.4 | −72.4 | 74.3 | 10 | A |
| LEU | CD1 | 46 | −10.3 | −71.2 | 73.9 | 9 | A |
| LEU | CD2 | 46 | −10.0 | −73.7 | 74.0 | 11 | A |
| LEU | C | 46 | −5.8 | −73.0 | 73.1 | 4 | A |
| LEU | O | 46 | −4.9 | −72.6 | 73.8 | 6 | A |
| ARG | N | 47 | −5.7 | −73.2 | 71.8 | 5 | A |
| ARG | CA | 47 | −4.5 | −72.9 | 71.0 | 8 | A |
| ARG | CB | 47 | −3.6 | −74.1 | 70.7 | 11 | A |
| ARG | CG | 47 | −2.7 | −74.4 | 71.8 | 18 | A |
| ARG | CD | 47 | −1.7 | −75.5 | 71.3 | 30 | A |
| ARC | NE | 47 | −2.3 | −76.6 | 70.6 | 38 | A |
| ARG | CZ | 47 | −1.7 | −77.6 | 70.1 | 42 | A |
| ARG | NH1 | 47 | −0.4 | −77.8 | 70.2 | 43 | A |
| ARG | NH2 | 47 | −2.4 | −78.6 | 69.5 | 44 | A |
| ARG | C | 47 | −5.0 | −72.3 | 69.7 | 7 | A |
| ARG | O | 47 | −6.2 | −72.6 | 69.3 | 5 | A |
| GLY | N | 48 | −4.2 | −71.6 | 68.9 | 8 | A |
| GLY | CA | 48 | −4.6 | −71.1 | 67.6 | 16 | A |
| GLY | C | 48 | −3.8 | −69.9 | 67.1 | 19 | A |
| GLY | O | 48 | −3.5 | −68.9 | 67.9 | 20 | A |
| LYS | N | 49 | −3.3 | −70.0 | 65.9 | 20 | A |
| LYS | CA | 49 | −2.5 | −69.0 | 65.3 | 21 | A |
| LYS | CB | 49 | −1.6 | −69.5 | 64.2 | 22 | A |
| LYS | CG | 49 | −2.3 | −70.6 | 63.3 | 26 | A |
| LYS | CD | 49 | −1.3 | −71.3 | 62.3 | 28 | A |
| LYS | CE | 49 | −1.2 | −70.5 | 61.0 | 32 | A |
| LYS | NZ | 49 | −2.4 | −70.8 | 60.1 | 30 | A |
| LYS | C | 49 | −3.3 | −67.8 | 64.8 | 21 | A |
| LYS | O | 49 | −4.1 | −67.9 | 63.9 | 24 | A |
| SER | N | 50 | −3.0 | −66.6 | 65.3 | 23 | A |
| SER | CA | 50 | −3.6 | −65.4 | 65.0 | 26 | A |
| SER | CB | 50 | −2.7 | −64.6 | 64.0 | 31 | A |
| SER | OG | 50 | −3.2 | −63.3 | 63.7 | 38 | A |
| SER | C | 50 | −5.1 | −65.4 | 64.3 | 26 | A |
| SER | O | 50 | −6.1 | −65.6 | 65.0 | 31 | A |
| GLY | N | 51 | −5.1 | −65.0 | 63.1 | 24 | A |
| GLY | CA | 51 | −6.4 | −65.0 | 62.4 | 19 | A |
| GLY | C | 51 | −7.0 | −66.3 | 61.9 | 19 | A |
| GLY | O | 51 | −7.8 | −66.4 | 61.0 | 21 | A |
| GLN | N | 52 | −6.5 | −67.4 | 62.5 | 19 | A |
| GLN | CA | 52 | −6.9 | −68.7 | 62.2 | 18 | A |
| GLN | CB | 52 | −5.7 | −69.7 | 62.2 | 16 | A |
| GLN | CG | 52 | −5.2 | −70.1 | 60.7 | 15 | A |
| GLN | CD | 52 | −4.5 | −69.0 | 60.0 | 20 | A |
| GLN | OE1 | 52 | −4.1 | −69.2 | 58.9 | 21 | A |
| GLN | NE2 | 52 | −4.4 | −67.8 | 60.6 | 21 | A |
| GLN | C | 52 | −7.9 | −69.3 | 63.2 | 18 | A |
| GLN | O | 52 | −8.7 | −70.2 | 62.8 | 17 | A |
| GLY | N | 53 | −8.0 | −68.7 | 64.3 | 18 | A |
| GLY | CA | 53 | −9.0 | −69.1 | 65.3 | 15 | A |
| GLY | C | 53 | −8.5 | −70.0 | 66.4 | 11 | A |
| GLY | O | 53 | −7.4 | −70.6 | 66.4 | 9 | A |
| TYR | N | 54 | −9.4 | −70.2 | 67.4 | 11 | A |
| TYR | CA | 54 | −9.0 | −70.9 | 68.6 | 8 | A |
| TYR | CB | 54 | −9.5 | −70.2 | 69.9 | 6 | A |
| TYR | CG | 54 | −8.9 | −68.8 | 70.0 | 2 | A |
| TYR | CD1 | 54 | −9.6 | −67.8 | 69.4 | 4 | A |
| TYR | CE1 | 54 | −9.0 | −66.5 | 69.5 | 6 | A |
| TYR | CD2 | 54 | −7.8 | −68.6 | 70.7 | 3 | A |
| TYR | CE2 | 54 | −7.3 | −67.3 | 70.8 | 5 | A |
| TYR | CZ | 54 | −7.9 | −66.3 | 70.2 | 6 | A |
| TYR | OH | 54 | −7.4 | −65.0 | 70.3 | 9 | A |
| TYR | C | 54 | −9.7 | −72.3 | 68.6 | 8 | A |
| TYR | O | 54 | −10.8 | −72.5 | 68.1 | 7 | A |
| TYR | N | 55 | −8.9 | −73.3 | 69.1 | 7 | A |
| TYR | CA | 55 | −9.3 | −74.7 | 69.1 | 5 | A |
| TYR | CB | 55 | −8.7 | −75.5 | 68.0 | 4 | A |
| TYR | CG | 55 | −7.2 | −75.5 | 67.9 | 5 | A |
| TYR | CD1 | 55 | −6.5 | −76.4 | 68.7 | 9 | A |
| TYR | CE1 | 55 | −5.1 | −76.5 | 68.7 | 4 | A |
| TYR | CD2 | 55 | −6.5 | −74.7 | 67.1 | 4 | A |
| TYR | CE2 | 55 | −5.1 | −74.7 | 67.0 | 1 | A |
| TYR | CZ | 55 | −4.4 | −75.6 | 67.8 | 1 | A |
| TYR | OH | 55 | −3.0 | −75.7 | 67.7 | 1 | A |
| TYR | C | 55 | −9.0 | −75.3 | 70.5 | 6 | A |
| TYR | O | 55 | −8.0 | −75.0 | 71.1 | 7 | A |
| VAL | N | 56 | −9.8 | −76.3 | 70.9 | 5 | A |
| VAL | CA | 56 | −9.6 | −77.0 | 72.2 | 6 | A |
| VAL | CB | 56 | −10.8 | −76.8 | 73.1 | 1 | A |
| VAL | CG1 | 56 | −12.1 | −77.4 | 72.5 | 1 | A |
| VAL | CG2 | 56 | −10.5 | −77.5 | 74.4 | 2 | A |
| VAL | C | 56 | −9.5 | −78.5 | 71.8 | 10 | A |
| VAL | O | 56 | −10.3 | −78.9 | 70.9 | 13 | A |
| GLU | N | 57 | −8.7 | −79.2 | 72.4 | 11 | A |
| GLU | CA | 57 | −8.5 | −80.6 | 72.1 | 9 | A |
| GLU | CB | 57 | −7.2 | −81.2 | 72.7 | 12 | A |
| GLU | CG | 57 | −7.0 | −82.6 | 72.5 | 15 | A |
| GLU | CD | 57 | −5.6 | −83.0 | 72.9 | 22 | A |
| GLU | OE1 | 57 | −4.6 | −82.7 | 72.1 | 27 | A |
| GLU | OE2 | 57 | −5.3 | −83.7 | 73.9 | 23 | A |
| GLU | C | 57 | −9.7 | −81.5 | 72.5 | 9 | A |
| GLU | O | 57 | −10.2 | −81.3 | 73.6 | 11 | A |
| MET | N | 58 | −10.2 | −82.3 | 71.6 | 6 | A |
| MET | CA | 58 | −11.3 | −83.2 | 71.9 | 7 | A |
| MET | CB | 58 | −12.6 | −82.7 | 71.2 | 2 | A |
| MET | CG | 58 | −13.2 | −81.4 | 71.8 | 6 | A |
| MET | SD | 58 | −14.6 | −80.8 | 70.8 | 4 | A |
| MET | CE | 58 | −16.0 | −81.4 | 71.8 | 2 | A |
| MET | C | 58 | −11.1 | −84.6 | 71.3 | 9 | A |
| MET | O | 58 | −10.2 | −84.8 | 70.5 | 13 | A |
| THR | N | 59 | −11.9 | −85.6 | 71.8 | 14 | A |
| THR | CA | 59 | −11.8 | −87.0 | 71.4 | 15 | A |
| THR | CB | 59 | −11.1 | −87.9 | 72.4 | 17 | A |
| THR | OG1 | 59 | −12.0 | −88.1 | 73.5 | 18 | A |
| THR | CG2 | 59 | −9.8 | −87.3 | 72.9 | 21 | A |
| THR | C | 59 | −13.1 | −87.5 | 71.0 | 15 | A |
| THR | O | 59 | −14.1 | −87.4 | 71.8 | 14 | A |
| VAL | N | 60 | −13.3 | −88.2 | 69.8 | 15 | A |
| VAL | CA | 60 | −14.5 | −88.7 | 69.4 | 14 | A |
| VAL | CB | 60 | −15.1 | −88.1 | 68.1 | 12 | A |
| VAL | CG1 | 60 | −15.2 | −86.6 | 68.4 | 12 | A |
| VAL | CG2 | 60 | −14.1 | −88.3 | 67.0 | 12 | A |
| VAL | C | 60 | −14.4 | −90.2 | 69.1 | 14 | A |
| VAL | O | 60 | −13.2 | −90.7 | 68.9 | 14 | A |
| GLY | N | 61 | −15.5 | −91.0 | 69.1 | 16 | A |
| GLY | CA | 61 | −15.5 | −92.4 | 68.9 | 19 | A |
| GLY | C | 61 | −14.7 | −93.3 | 69.8 | 20 | A |
| GLY | O | 61 | −13.9 | −92.8 | 70.7 | 24 | A |
| SER | N | 62 | −14.9 | −94.6 | 69.7 | 19 | A |
| SER | CA | 62 | −14.2 | −95.5 | 70.6 | 18 | A |
| SER | CB | 62 | −15.2 | −96.3 | 71.4 | 15 | A |
| SER | OG | 62 | −15.9 | −95.4 | 72.3 | 19 | A |
| SER | C | 62 | −13.4 | −96.6 | 69.8 | 20 | A |
| SER | O | 62 | −14.0 | −97.2 | 68.9 | 24 | A |
| PRO | N | 63 | −12.1 | −96.7 | 70.0 | 18 | A |
| PRO | CD | 63 | −11.2 | −97.6 | 69.2 | 16 | A |
| PRO | CA | 63 | −11.3 | −96.0 | 71.0 | 18 | A |
| PRO | CB | 63 | −9.9 | −96.7 | 70.9 | 19 | A |
| PRO | CG | 63 | −9.9 | −97.0 | 69.4 | 18 | A |
| PRO | C | 63 | −11.2 | −94.5 | 70.6 | 19 | A |
| PRO | O | 63 | −11.4 | −94.1 | 69.5 | 20 | A |
| PRO | N | 64 | −10.8 | −93.7 | 71.6 | 20 | A |
| PRO | CD | 64 | −10.5 | −94.0 | 73.0 | 19 | A |
| PRO | CA | 64 | −10.6 | −92.2 | 71.4 | 19 | A |
| PRO | CB | 64 | −10.2 | −91.7 | 72.8 | 19 | A |
| PRO | CG | 64 | −10.8 | −92.8 | 73.7 | 19 | A |
| PRO | C | 64 | −9.7 | −91.7 | 70.3 | 19 | A |
| PRO | O | 64 | −8.5 | −92.0 | 70.3 | 21 | A |
| GLN | N | 65 | −10.3 | −90.9 | 69.4 | 18 | A |
| GLN | CA | 65 | −9.6 | −90.2 | 68.3 | 18 | A |
| GLN | CB | 65 | −10.4 | −90.3 | 67.0 | 18 | A |
| GLN | CG | 65 | −10.4 | −91.6 | 66.4 | 17 | A |
| GLN | CD | 65 | −10.8 | −91.6 | 64.9 | 19 | A |
| GLN | OE1 | 65 | −10.2 | −90.9 | 64.1 | 18 | A |
| GLN | NE2 | 65 | −11.9 | −92.3 | 64.6 | 22 | A |
| GLN | C | 65 | −9.4 | −88.7 | 68.7 | 18 | A |
| GLN | O | 65 | −10.3 | −88.0 | 68.8 | 18 | A |
| THR | N | 66 | −8.1 | −88.4 | 68.9 | 16 | A |
| THR | CA | 66 | −7.8 | −87.0 | 69.2 | 13 | A |
| THR | CB | 66 | −6.3 | −86.9 | 69.8 | 13 | A |
| THR | OG1 | 66 | −6.2 | −87.8 | 70.9 | 13 | A |
| THR | CG2 | 66 | −6.0 | −85.5 | 70.2 | 11 | A |
| THR | C | 66 | −7.9 | −86.1 | 68.0 | 11 | A |
| THR | O | 66 | −7.5 | −86.4 | 66.9 | 11 | A |
| LEU | N | 67 | −8.6 | −84.9 | 68.2 | 11 | A |
| LEU | CA | 67 | −8.8 | −83.9 | 67.2 | 10 | A |
| LEU | CB | 67 | −10.1 | −84.2 | 66.4 | 13 | A |
| LEU | CG | 67 | −10.1 | −85.4 | 65.4 | 15 | A |
| LEU | CD1 | 67 | −11.5 | −85.7 | 65.0 | 15 | A |
| LEU | CD2 | 67 | −9.2 | −85.0 | 64.2 | 14 | A |
| LEU | C | 67 | −8.9 | −82.5 | 67.8 | 10 | A |
| LEU | O | 67 | −9.5 | −82.3 | 68.8 | 7 | A |
| ASN | N | 68 | −8.4 | −81.5 | 67.0 | 9 | A |
| ASN | CA | 68 | −8.5 | −80.2 | 67.5 | 9 | A |
| ASN | CB | 68 | −7.2 | −79.4 | 67.0 | 8 | A |
| ASN | CG | 68 | −6.0 | −79.8 | 67.9 | 6 | A |
| ASN | OD1 | 68 | −4.9 | −79.4 | 67.6 | 11 | A |
| ASN | ND2 | 68 | −6.2 | −80.5 | 69.0 | 10 | A |
| ASN | C | 68 | −9.7 | −79.5 | 66.9 | 7 | A |
| ASN | O | 68 | −9.8 | −79.3 | 65.7 | 8 | A |
| ILE | N | 69 | −10.6 | −79.1 | 67.7 | 6 | A |
| ILE | CA | 69 | −11.9 | −78.4 | 67.3 | 7 | A |
| ILE | CB | 69 | −13.1 | −79.0 | 68.0 | 7 | A |
| ILE | CG2 | 69 | −14.3 | −78.5 | 67.4 | 13 | A |
| ILE | CG1 | 69 | −13.0 | −80.5 | 67.8 | 13 | A |
| ILE | CD1 | 69 | −12.9 | −81.0 | 66.3 | 14 | A |
| ILE | C | 69 | −11.9 | −76.9 | 67.5 | 10 | A |
| ILE | O | 69 | −11.6 | −76.4 | 68.6 | 14 | A |
| LEU | N | 70 | −12.4 | −76.2 | 66.5 | 8 | A |
| LEU | CA | 70 | −12.5 | −74.7 | 66.5 | 6 | A |
| LEU | CB | 70 | −12.8 | −74.3 | 65.1 | 6 | A |
| LEU | CG | 70 | −12.8 | −72.8 | 64.7 | 4 | A |
| LEU | CD1 | 70 | −11.4 | −72.4 | 64.2 | 3 | A |
| LEU | CD2 | 70 | −13.9 | −72.5 | 63.7 | 2 | A |
| LEU | C | 70 | −13.6 | −74.3 | 67.4 | 5 | A |
| LEU | O | 70 | −14.7 | −74.8 | 67.3 | 7 | A |
| VAL | N | 71 | −13.3 | −73.4 | 68.4 | 1 | A |
| VAL | CA | 71 | −14.3 | −73.0 | 69.3 | 2 | A |
| VAL | CB | 71 | −13.7 | −72.5 | 70.6 | 4 | A |
| VAL | CG1 | 71 | −14.8 | −72.2 | 71.6 | 5 | A |
| VAL | CG2 | 71 | −12.7 | −73.5 | 71.1 | 5 | A |
| VAL | C | 71 | −15.0 | −71.8 | 68.6 | 2 | A |
| VAL | O | 71 | −14.4 | −70.7 | 68.4 | 6 | A |
| ASP | N | 72 | −16.2 | −72.0 | 68.2 | 1 | A |
| ASP | CA | 72 | −17.0 | −71.0 | 67.4 | 1 | A |
| ASP | CB | 72 | −17.2 | −71.5 | 66.0 | 4 | A |
| ASP | CG | 72 | −18.1 | −70.6 | 65.1 | 9 | A |
| ASP | OD1 | 72 | −18.3 | −69.4 | 65.5 | 13 | A |
| ASP | OD2 | 72 | −18.6 | −71.1 | 64.1 | 7 | A |
| ASP | C | 72 | −18.4 | −70.8 | 68.0 | 1 | A |
| ASP | O | 72 | −19.3 | −71.6 | 68.0 | 1 | A |
| THR | N | 73 | −18.6 | −69.6 | 68.6 | 1 | A |
| THR | CA | 73 | −19.8 | −69.3 | 69.2 | 2 | A |
| THR | CB | 73 | −19.6 | −68.3 | 70.4 | 3 | A |
| THR | OG1 | 73 | −18.8 | −67.2 | 70.0 | 6 | A |
| THR | CG2 | 73 | −18.9 | −68.9 | 71.6 | 2 | A |
| THR | C | 73 | −20.8 | −68.6 | 68.2 | 1 | A |
| THR | O | 73 | −21.7 | −67.9 | 68.6 | 1 | A |
| GLY | N | 74 | −20.5 | −68.9 | 66.9 | 1 | A |
| GLY | CA | 74 | −21.3 | −68.3 | 65.9 | 3 | A |
| GLY | C | 74 | −22.1 | −69.3 | 65.1 | 1 | A |
| GLY | O | 74 | −22.7 | −69.0 | 64.0 | 1 | A |
| SER | N | 75 | −22.1 | −70.5 | 65.5 | 2 | A |
| SER | CA | 75 | −22.8 | −71.6 | 64.8 | 1 | A |
| SER | CB | 75 | −21.9 | −72.2 | 63.7 | 1 | A |
| SER | OG | 75 | −20.9 | −73.0 | 64.3 | 5 | A |
| SER | C | 75 | −23.2 | −72.7 | 65.8 | 1 | A |
| SER | O | 75 | −22.8 | −72.6 | 67.0 | 1 | A |
| SER | N | 76 | −24.0 | −73.7 | 65.4 | 2 | A |
| SER | CA | 76 | −24.4 | −74.7 | 66.4 | 5 | A |
| SER | CB | 76 | −25.8 | −74.4 | 66.8 | 5 | A |
| SER | OG | 76 | −25.9 | −73.4 | 67.7 | 6 | A |
| SER | C | 76 | −24.2 | −76.1 | 65.9 | 4 | A |
| SER | O | 76 | −24.9 | −77.0 | 66.3 | 5 | A |
| ASN | N | 77 | −23.2 | −76.3 | 65.1 | 5 | A |
| ASN | CA | 77 | −23.0 | −77.7 | 64.6 | 6 | A |
| ASN | CB | 77 | −23.1 | −77.8 | 63.1 | 8 | A |
| ASN | CG | 77 | −24.5 | −77.6 | 62.6 | 7 | A |
| ASN | OD1 | 77 | −25.0 | −76.4 | 62.6 | 8 | A |
| ASN | ND2 | 77 | −25.2 | −78.6 | 62.2 | 6 | A |
| ASN | C | 77 | −21.6 | −78.1 | 65.1 | 5 | A |
| ASN | O | 77 | −20.7 | −77.3 | 65.1 | 3 | A |
| PHE | N | 78 | −21.5 | −79.4 | 65.5 | 6 | A |
| PHE | CA | 78 | −20.3 | −80.0 | 65.9 | 6 | A |
| PHE | CB | 78 | −20.5 | −80.9 | 67.1 | 6 | A |
| PHE | CG | 78 | −19.2 | −81.6 | 67.5 | 8 | A |
| PHE | CD1 | 78 | −19.3 | −82.7 | 68.3 | 6 | A |
| PHE | CD2 | 78 | −18.0 | −81.2 | 67.1 | 10 | A |
| PHE | CE1 | 78 | −18.2 | −83.5 | 68.7 | 4 | A |
| PHE | CE2 | 78 | −16.8 | −81.9 | 67.5 | 5 | A |
| PHE | CZ | 78 | −16.9 | −83.0 | 68.3 | 3 | A |
| PHE | C | 78 | −19.9 | −80.8 | 64.7 | 7 | A |
| PHE | O | 78 | −20.6 | −81.8 | 64.4 | 6 | A |
| ALA | N | 79 | −18.9 | −80.4 | 63.9 | 7 | A |
| ALA | CA | 79 | −18.5 | −81.1 | 62.7 | 8 | A |
| ALA | CB | 79 | −19.0 | −80.4 | 61.5 | 7 | A |
| ALA | C | 79 | −17.0 | −81.3 | 62.7 | 12 | A |
| ALA | O | 79 | −16.3 | −80.6 | 63.4 | 11 | A |
| VAL | N | 80 | −16.6 | −82.3 | 61.9 | 13 | A |
| VAL | CA | 80 | −15.1 | −82.6 | 61.8 | 13 | A |
| VAL | CB | 80 | −14.7 | −83.6 | 62.9 | 16 | A |
| VAL | CG1 | 80 | −15.1 | −83.1 | 64.3 | 15 | A |
| VAL | CG2 | 80 | −15.3 | −85.0 | 62.7 | 16 | A |
| VAL | C | 80 | −14.8 | −83.2 | 60.5 | 13 | A |
| VAL | O | 80 | −15.7 | −83.7 | 59.8 | 9 | A |
| GLY | N | 81 | −13.5 | −83.1 | 60.1 | 12 | A |
| GLY | CA | 81 | −13.1 | −83.7 | 58.8 | 16 | A |
| GLY | C | 81 | −13.3 | −85.2 | 58.9 | 20 | A |
| GLY | O | 81 | −12.8 | −85.8 | 59.9 | 21 | A |
| ALA | N | 82 | −13.8 | −85.8 | 57.9 | 22 | A |
| ALA | CA | 82 | −14.0 | −87.3 | 57.9 | 20 | A |
| ALA | CB | 82 | −15.5 | −87.6 | 58.0 | 20 | A |
| ALA | C | 82 | −13.4 | −88.0 | 56.7 | 21 | A |
| ALA | O | 82 | −14.0 | −89.0 | 56.2 | 23 | A |
| ALA | N | 83 | −12.4 | −87.4 | 56.1 | 20 | A |
| ALA | CA | 83 | −11.7 | −88.0 | 54.9 | 19 | A |
| ALA | CB | 83 | −12.7 | −88.1 | 53.8 | 18 | A |
| ALA | C | 83 | −10.6 | −87.1 | 54.5 | 21 | A |
| ALA | O | 83 | −10.8 | −85.9 | 54.4 | 23 | A |
| PRO | N | 84 | −9.4 | −87.7 | 54.2 | 21 | A |
| PRO | CD | 84 | −9.3 | −89.0 | 53.7 | 20 | A |
| PRO | CA | 84 | −8.2 | −86.9 | 53.8 | 23 | A |
| PRO | CB | 84 | −7.4 | −87.9 | 53.1 | 20 | A |
| PRO | CG | 84 | −8.4 | −88.9 | 52.5 | 23 | A |
| PRO | C | 84 | −8.5 | −85.7 | 52.9 | 25 | A |
| PRO | O | 84 | −9.2 | −85.7 | 52.0 | 27 | A |
| HIS | N | 85 | −7.8 | −84.6 | 53.3 | 25 | A |
| HIS | CA | 85 | −8.0 | −83.3 | 52.6 | 25 | A |
| HIS | CB | 85 | −9.0 | −82.4 | 53.3 | 25 | A |
| HIS | CG | 85 | −9.1 | −81.0 | 52.7 | 25 | A |
| HIS | CD2 | 85 | −10.1 | −80.4 | 52.2 | 27 | A |
| HIS | ND1 | 85 | −8.0 | −80.2 | 52.6 | 25 | A |
| HIS | CE1 | 85 | −8.4 | −79.0 | 52.1 | 25 | A |
| HIS | NE2 | 85 | −9.7 | −79.1 | 51.8 | 28 | A |
| HIS | C | 85 | −6.6 | −82.6 | 52.6 | 25 | A |
| HIS | O | 85 | −5.9 | −82.7 | 53.6 | 26 | A |
| PRO | N | 86 | −6.2 | −82.1 | 51.4 | 25 | A |
| PRO | CD | 86 | −7.1 | −81.9 | 50.2 | 25 | A |
| PRO | CA | 86 | −4.9 | −81.4 | 51.2 | 27 | A |
| PRO | CB | 86 | −5.2 | −80.4 | 50.1 | 27 | A |
| PRO | CG | 86 | −6.1 | −81.3 | 49.2 | 27 | A |
| PRO | C | 86 | −4.3 | −80.7 | 52.4 | 29 | A |
| PRO | O | 86 | −3.1 | −80.6 | 52.5 | 29 | A |
| PHE | N | 87 | −5.2 | −80.2 | 53.3 | 26 | A |
| PHE | CA | 87 | −4.7 | −79.4 | 54.5 | 24 | A |
| PHE | CB | 87 | −5.5 | −78.1 | 54.6 | 25 | A |
| PHE | CG | 87 | −5.2 | −77.2 | 53.5 | 24 | A |
| PHE | CD1 | 87 | −5.9 | −76.0 | 53.3 | 28 | A |
| PHE | CD2 | 87 | −4.2 | −77.5 | 52.5 | 27 | A |
| PHE | CE1 | 87 | −5.7 | −75.1 | 52.3 | 31 | A |
| PHE | CE2 | 87 | −3.9 | −76.6 | 51.5 | 29 | A |
| PHE | CZ | 87 | −4.7 | −75.4 | 51.3 | 32 | A |
| PHE | C | 87 | −4.8 | −80.2 | 55.8 | 20 | A |
| PHE | O | 87 | −3.9 | −80.1 | 56.6 | 17 | A |
| LEU | N | 88 | −5.8 | −81.0 | 55.9 | 16 | A |
| LEU | CA | 88 | −6.0 | −81.9 | 57.1 | 18 | A |
| LEU | CB | 88 | −7.3 | −82.7 | 57.0 | 17 | A |
| LEU | CG | 88 | −8.6 | −81.9 | 57.2 | 18 | A |
| LEU | CD1 | 88 | −9.8 | −82.9 | 57.2 | 19 | A |
| LEU | CD2 | 88 | −8.6 | −81.1 | 58.5 | 19 | A |
| LEU | C | 88 | −4.9 | −82.8 | 57.4 | 21 | A |
| LEU | O | 88 | −4.5 | −83.7 | 56.6 | 21 | A |
| HIS | N | 89 | −4.3 | −82.6 | 58.6 | 21 | A |
| HIS | CA | 89 | −3.2 | −83.4 | 59.1 | 22 | A |
| HIS | CB | 89 | −2.5 | −82.8 | 60.3 | 27 | A |
| HIS | CG | 89 | −1.7 | −81.5 | 60.0 | 35 | A |
| HIS | CD2 | 89 | −1.6 | −80.3 | 60.6 | 36 | A |
| HIS | ND1 | 89 | −0.8 | −81.5 | 58.9 | 39 | A |
| HIS | CE1 | 89 | −0.2 | −80.3 | 58.9 | 39 | A |
| HIS | NE2 | 89 | −0.7 | −79.6 | 59.9 | 40 | A |
| HIS | C | 89 | −3.7 | −84.8 | 59.4 | 20 | A |
| HIS | O | 89 | −3.0 | −85.8 | 59.3 | 22 | A |
| ARG | N | 90 | −4.9 | −84.8 | 59.9 | 16 | A |
| ARG | CA | 90 | −5.6 | −86.1 | 60.3 | 13 | A |
| ARG | CB | 90 | −5.2 | −86.4 | 61.7 | 14 | A |
| ARG | CG | 90 | −5.4 | −85.4 | 62.8 | 18 | A |
| ARG | CD | 90 | −5.2 | −85.9 | 64.2 | 20 | A |
| ARG | NE | 90 | −5.1 | −84.9 | 65.2 | 23 | A |
| ARG | CZ | 90 | −4.9 | −85.1 | 66.5 | 22 | A |
| ARG | NH1 | 90 | −4.6 | −86.4 | 66.9 | 16 | A |
| ARG | NH2 | 90 | −4.9 | −84.1 | 67.4 | 20 | A |
| ARG | C | 90 | −7.1 | −85.9 | 60.2 | 12 | A |
| ARG | O | 90 | −7.6 | −84.8 | 59.9 | 15 | A |
| TYR | N | 91 | −7.9 | −86.9 | 60.4 | 11 | A |
| TYR | CA | 91 | −9.3 | −86.8 | 60.3 | 13 | A |
| TYR | CB | 91 | −9.8 | −86.9 | 58.9 | 18 | A |
| TYR | CG | 91 | −9.5 | −88.1 | 58.1 | 24 | A |
| TYR | CD1 | 91 | −10.4 | −89.2 | 58.2 | 24 | A |
| TYR | CE2 | 91 | −10.1 | −90.4 | 57.5 | 27 | A |
| TYR | CD2 | 91 | −8.4 | −88.3 | 57.3 | 23 | A |
| TYR | CE2 | 91 | −8.1 | −89.5 | 56.6 | 24 | A |
| TYR | CZ | 91 | −9.0 | −90.5 | 56.7 | 28 | A |
| TYR | OH | 91 | −8.8 | −91.7 | 56.0 | 32 | A |
| TYR | C | 91 | −10.0 | −87.9 | 61.2 | 13 | A |
| TYR | O | 91 | −9.4 | −88.7 | 61.8 | 14 | A |
| TYR | N | 92 | −11.4 | −87.8 | 61.1 | 15 | A |
| TYR | CA | 92 | −12.2 | −88.7 | 61.9 | 15 | A |
| TYR | CB | 92 | −13.5 | −88.0 | 62.3 | 10 | A |
| TYR | CG | 92 | −14.5 | −88.9 | 63.0 | 6 | A |
| TYR | CD1 | 92 | −14.2 | −89.8 | 64.0 | 6 | A |
| TYR | CE1 | 92 | −15.2 | −90.5 | 64.6 | 6 | A |
| TYR | CD2 | 92 | −15.9 | −88.7 | 62.7 | 3 | A |
| TYR | CE2 | 92 | −16.9 | −89.4 | 63.4 | 6 | A |
| TYR | CZ | 92 | −16.5 | −90.3 | 64.3 | 4 | A |
| TYR | OH | 92 | −17.5 | −91.0 | 65.0 | 1 | A |
| TYR | C | 92 | −12.5 | −90.0 | 61.2 | 17 | A |
| TYR | O | 92 | −13.3 | −90.0 | 60.2 | 21 | A |
| GLN | N | 93 | −12.0 | −91.1 | 61.7 | 18 | A |
| GLN | CA | 93 | −12.2 | −92.4 | 61.0 | 18 | A |
| GLN | CB | 93 | −11.0 | −93.3 | 61.2 | 19 | A |
| GLN | CG | 93 | −9.8 | −92.8 | 60.4 | 21 | A |
| GLN | CD | 93 | −8.5 | −93.6 | 60.7 | 22 | A |
| GLN | OE1 | 93 | −8.1 | −93.8 | 61.9 | 23 | A |
| GLN | NE2 | 93 | −7.9 | −94.2 | 59.6 | 22 | A |
| GLN | C | 93 | −13.4 | −93.1 | 61.7 | 16 | A |
| GLN | O | 93 | −13.3 | −93.6 | 62.8 | 12 | A |
| ARG | N | 94 | −14.6 | −93.0 | 61.1 | 16 | A |
| ARG | CA | 94 | −15.8 | −93.6 | 61.6 | 20 | A |
| ARG | CB | 94 | −17.0 | −93.2 | 60.7 | 21 | A |
| ARG | CG | 94 | −17.3 | −91.7 | 60.8 | 22 | A |
| ARG | CD | 94 | −18.4 | −91.4 | 59.8 | 18 | A |
| ARG | NE | 94 | −17.9 | −91.0 | 58.5 | 24 | A |
| ARG | CZ | 94 | −18.7 | −90.7 | 57.4 | 26 | A |
| ARG | NH1 | 94 | −20.0 | −90.7 | 57.6 | 24 | A |
| ARG | NH2 | 94 | −18.1 | −90.4 | 56.2 | 29 | A |
| ARG | C | 94 | −15.7 | −95.1 | 61.7 | 19 | A |
| ARG | O | 94 | −16.0 | −95.7 | 62.7 | 16 | A |
| GLN | N | 95 | −15.3 | −95.7 | 60.6 | 22 | A |
| GLN | CA | 95 | −15.1 | −97.2 | 60.5 | 23 | A |
| GLN | CB | 95 | −14.5 | −97.6 | 59.2 | 27 | A |
| GLN | CG | 95 | −15.3 | −97.2 | 58.0 | 31 | A |
| GLN | CD | 95 | −14.6 | −97.8 | 56.7 | 36 | A |
| GLN | OE1 | 95 | −14.6 | −99.0 | 56.5 | 35 | A |
| GLN | NE2 | 95 | −14.1 | −96.9 | 55.9 | 37 | A |
| GLN | C | 95 | −14.4 | −97.8 | 61.7 | 24 | A |
| GLN | O | 95 | −14.6 | −98.9 | 62.1 | 24 | A |
| LEU | N | 96 | −13.4 | −97.0 | 62.3 | 21 | A |
| LEU | CA | 96 | −12.6 | −97.5 | 63.4 | 19 | A |
| LEU | CB | 96 | −11.3 | −96.9 | 63.3 | 18 | A |
| LEU | CG | 96 | −10.3 | −97.5 | 62.3 | 15 | A |
| LEU | CD1 | 96 | −9.1 | −96.6 | 62.0 | 16 | A |
| LEU | CD2 | 96 | −9.8 | −98.9 | 62.9 | 15 | A |
| LEU | C | 96 | −13.3 | −97.3 | 64.8 | 19 | A |
| LEU | O | 96 | −12.7 | −97.6 | 65.8 | 20 | A |
| SER | N | 97 | −14.5 | −96.8 | 64.8 | 21 | A |
| SER | CA | 97 | −15.2 | −96.5 | 66.1 | 22 | A |
| SER | CB | 97 | −15.8 | −95.1 | 66.2 | 27 | A |
| SER | OG | 97 | −16.6 | −95.0 | 67.3 | 27 | A |
| SER | C | 97 | −16.3 | −97.5 | 66.3 | 22 | A |
| SER | O | 97 | −17.0 | −97.9 | 65.4 | 22 | A |
| SER | N | 98 | −16.4 | −98.0 | 67.5 | 23 | A |
| SER | CA | 98 | −17.4 | −99.0 | 67.9 | 24 | A |
| SER | CB | 98 | −17.0 | −99.9 | 69.0 | 24 | A |
| SER | OG | 98 | −16.6 | −99.1 | 70.1 | 26 | A |
| SER | C | 98 | −18.7 | −98.3 | 68.2 | 24 | A |
| SER | O | 98 | −19.8 | −98.8 | 67.9 | 24 | A |
| THR | N | 99 | −18.6 | −97.2 | 68.9 | 22 | A |
| THR | CA | 99 | −19.7 | −96.4 | 69.4 | 19 | A |
| THR | CB | 99 | −19.4 | −95.6 | 70.6 | 17 | A |
| THR | OG1 | 99 | −18.2 | −94.8 | 70.3 | 16 | A |
| THR | CG2 | 99 | −19.1 | −96.5 | 71.8 | 19 | A |
| THR | C | 99 | −20.3 | −95.4 | 68.4 | 19 | A |
| THR | O | 99 | −21.1 | −94.6 | 68.7 | 21 | A |
| TYR | N | 100 | −19.9 | −95.6 | 67.1 | 17 | A |
| TYR | CA | 100 | −20.4 | −94.7 | 66.0 | 17 | A |
| TYR | CB | 100 | −19.5 | −94.8 | 64.8 | 17 | A |
| TYR | CG | 100 | −20.0 | −93.9 | 63.6 | 18 | A |
| TYR | CD1 | 100 | −19.9 | −92.6 | 63.6 | 20 | A |
| TYR | CE1 | 100 | −20.3 | −91.8 | 62.5 | 21 | A |
| TYR | CD2 | 100 | −20.5 | −94.6 | 62.5 | 19 | A |
| TYR | CE2 | 100 | −21.0 | −93.8 | 61.4 | 21 | A |
| TYR | CZ | 100 | −20.8 | −92.4 | 61.4 | 23 | A |
| TYR | OH | 100 | −21.2 | −91.7 | 60.3 | 22 | A |
| TYR | C | 100 | −21.8 | −95.1 | 65.6 | 17 | A |
| TYR | O | 100 | −22.3 | −96.2 | 65.8 | 18 | A |
| ARG | N | 101 | −22.5 | −94.1 | 65.1 | 16 | A |
| ARG | CA | 101 | −23.9 | −94.3 | 64.6 | 18 | A |
| ARG | CB | 101 | −24.9 | −93.9 | 65.7 | 14 | A |
| ARG | CG | 101 | −25.3 | −95.0 | 66.6 | 15 | A |
| ARG | CD | 101 | −26.5 | −94.6 | 67.5 | 21 | A |
| ARG | NE | 101 | −27.0 | −95.6 | 68.4 | 26 | A |
| ARG | CZ | 101 | −26.2 | −96.2 | 69.3 | 29 | A |
| ARG | NH1 | 101 | −25.0 | −95.9 | 69.4 | 28 | A |
| ARG | NH2 | 101 | −26.8 | −97.2 | 70.1 | 29 | A |
| ARG | C | 101 | −24.0 | −93.5 | 63.3 | 20 | A |
| ARG | O | 101 | −23.1 | −92.7 | 63.0 | 21 | A |
| ASP | N | 102 | −25.1 | −93.6 | 62.6 | 19 | A |
| ASP | CA | 102 | −25.3 | −92.9 | 61.4 | 20 | A |
| ASP | CB | 102 | −24.7 | −93.7 | 60.3 | 17 | A |
| ASP | CG | 102 | −25.1 | −93.2 | 58.9 | 18 | A |
| ASP | OD1 | 102 | −24.9 | −92.1 | 58.5 | 19 | A |
| ASP | OD2 | 102 | −25.6 | −94.1 | 58.1 | 18 | A |
| ASP | C | 102 | −26.8 | −92.6 | 61.2 | 21 | A |
| ASP | O | 102 | −27.5 | −93.5 | 60.9 | 25 | A |
| LEU | N | 103 | −27.2 | −91.4 | 61.4 | 18 | A |
| LEU | CA | 103 | −28.6 | −91.0 | 61.2 | 17 | A |
| LEU | CB | 103 | −29.0 | −89.7 | 61.9 | 16 | A |
| LEU | CG | 103 | −28.2 | −89.4 | 63.2 | 13 | A |
| LEU | CD1 | 103 | −27.2 | −88.3 | 62.9 | 10 | A |
| LEU | CD2 | 103 | −29.2 | −88.9 | 64.3 | 11 | A |
| LEU | C | 103 | −29.1 | −91.0 | 59.8 | 18 | A |
| LEU | O | 103 | −30.2 | −90.4 | 59.5 | 19 | A |
| ARG | N | 104 | −28.3 | −91.5 | 58.9 | 18 | A |
| ARG | CA | 104 | −28.6 | −91.6 | 57.4 | 21 | A |
| ARG | CB | 104 | −29.6 | −92.7 | 57.1 | 24 | A |
| ARG | CG | 104 | −28.9 | −94.1 | 57.1 | 28 | A |
| ARG | CD | 104 | −29.9 | −95.3 | 57.1 | 31 | A |
| ARG | NE | 104 | −31.1 | −95.1 | 56.2 | 35 | A |
| ARG | CZ | 104 | −32.3 | −94.8 | 56.7 | 35 | A |
| ARG | NH1 | 104 | −32.5 | −94.6 | 58.0 | 37 | A |
| ARG | NH2 | 104 | −33.3 | −94.6 | 55.8 | 32 | A |
| ARG | C | 104 | −29.3 | −90.3 | 57.0 | 21 | A |
| ARG | O | 104 | −30.4 | −90.3 | 56.5 | 24 | A |
| LYS | N | 105 | −28.6 | −89.2 | 57.2 | 21 | A |
| LYS | CA | 105 | −29.1 | −87.8 | 56.8 | 22 | A |
| LYS | CB | 105 | −30.0 | −87.3 | 57.8 | 20 | A |
| LYS | CG | 105 | −30.7 | −85.9 | 57.5 | 23 | A |
| LYS | CD | 105 | −31.8 | −85.5 | 58.4 | 26 | A |
| LYS | CE | 105 | −31.3 | −85.3 | 59.9 | 21 | A |
| LYS | NZ | 105 | −32.3 | −84.6 | 60.8 | 15 | A |
| LYS | C | 105 | −27.9 | −86.9 | 56.6 | 23 | A |
| LYS | O | 105 | −27.2 | −86.6 | 57.5 | 25 | A |
| GLY | N | 106 | −27.8 | −86.3 | 55.4 | 23 | A |
| GLY | CA | 106 | −26.7 | −85.4 | 55.1 | 22 | A |
| GLY | C | 106 | −27.0 | −84.0 | 55.6 | 21 | A |
| GLY | O | 106 | −28.1 | −83.7 | 56.1 | 20 | A |
| VAL | N | 107 | −26.0 | −83.1 | 55.5 | 21 | A |
| VAL | CA | 107 | −26.1 | −81.8 | 56.0 | 18 | A |
| VAL | CB | 107 | −25.9 | −81.7 | 57.5 | 11 | A |
| VAL | CG1 | 107 | −24.4 | −81.9 | 57.8 | 10 | A |
| VAL | CG2 | 107 | −26.3 | −80.3 | 58.0 | 7 | A |
| VAL | C | 107 | −25.2 | −80.8 | 55.2 | 19 | A |
| VAL | O | 107 | −24.0 | −81.2 | 54.9 | 16 | A |
| TYR | N | 108 | −25.6 | −79.6 | 54.9 | 20 | A |
| TYR | CA | 108 | −24.9 | −78.6 | 54.2 | 20 | A |
| TYR | CB | 108 | −25.5 | −78.3 | 52.9 | 21 | A |
| TYR | CG | 108 | −25.1 | −76.9 | 52.4 | 23 | A |
| TYR | CD1 | 108 | −23.8 | −76.5 | 52.3 | 24 | A |
| TYR | CE1 | 108 | −23.5 | −75.2 | 51.8 | 26 | A |
| TYR | CD2 | 108 | −26.1 | −76.0 | 52.0 | 22 | A |
| TYR | CE2 | 108 | −25.8 | −74.7 | 51.5 | 23 | A |
| TYR | CZ | 108 | −24.5 | −74.3 | 51.4 | 24 | A |
| TYR | OH | 108 | −24.1 | −73.1 | 51.0 | 23 | A |
| TYR | C | 108 | −24.8 | −77.4 | 55.1 | 21 | A |
| TYR | O | 108 | −25.9 | −76.8 | 55.4 | 21 | A |
| VAL | N | 109 | −23.7 | −77.0 | 55.6 | 18 | A |
| VAL | CA | 109 | −23.6 | −75.8 | 56.4 | 19 | A |
| VAL | CB | 109 | −23.0 | −76.2 | 57.8 | 19 | A |
| VAL | CG1 | 109 | −22.8 | −74.9 | 58.7 | 15 | A |
| VAL | CG2 | 109 | −24.0 | −77.1 | 58.5 | 19 | A |
| VAL | C | 109 | −22.6 | −74.7 | 55.8 | 21 | A |
| VAL | O | 109 | −21.4 | −74.9 | 55.8 | 23 | A |
| PRO | N | 110 | −23.2 | −73.6 | 55.4 | 23 | A |
| PRO | CD | 110 | −24.7 | −73.4 | 55.1 | 22 | A |
| PRO | CA | 110 | −22.4 | −72.5 | 54.8 | 24 | A |
| PRO | CB | 110 | −23.4 | −72.0 | 53.7 | 23 | A |
| PRO | CG | 110 | −24.7 | −72.0 | 54.4 | 23 | A |
| PRO | C | 110 | −22.1 | −71.5 | 55.9 | 24 | A |
| PRO | O | 110 | −22.7 | −71.4 | 56.9 | 21 | A |
| TYR | N | 111 | −21.1 | −70.6 | 55.6 | 25 | A |
| TYR | CA | 111 | −20.7 | −69.6 | 56.5 | 24 | A |
| TYR | CB | 111 | −19.4 | −70.0 | 57.3 | 20 | A |
| TYR | CG | 111 | −19.6 | −71.3 | 58.1 | 14 | A |
| TYR | CD1 | 111 | −20.1 | −71.2 | 59.4 | 14 | A |
| TYR | CE1 | 111 | −20.2 | −72.3 | 60.2 | 17 | A |
| TYR | CD2 | 111 | −19.3 | −72.5 | 57.6 | 15 | A |
| TYR | CE2 | 111 | −19.4 | −73.7 | 58.4 | 15 | A |
| TYR | CZ | 111 | −19.9 | −73.6 | 59.7 | 14 | A |
| TYR | OH | 111 | −19.9 | −74.7 | 60.5 | 14 | A |
| TYR | C | 111 | −20.4 | −68.3 | 55.7 | 24 | A |
| TYR | O | 111 | −20.4 | −68.3 | 54.4 | 21 | A |
| THR | N | 112 | −20.0 | −67.2 | 56.4 | 24 | A |
| THR | CA | 112 | −19.6 | −66.0 | 55.7 | 25 | A |
| THR | CB | 112 | −19.1 | −64.9 | 56.8 | 26 | A |
| THR | OG1 | 112 | −20.3 | −64.3 | 57.4 | 28 | A |
| THR | CG2 | 112 | −18.3 | −63.8 | 56.1 | 24 | A |
| THR | C | 112 | −18.5 | −66.3 | 54.8 | 25 | A |
| THR | O | 112 | −18.6 | −66.0 | 53.6 | 28 | A |
| GLN | N | 113 | −17.5 | −67.0 | 55.3 | 24 | A |
| GLN | CA | 113 | −16.4 | −67.4 | 54.6 | 26 | A |
| GLN | CB | 113 | −15.1 | −66.7 | 55.1 | 27 | A |
| GLN | CG | 113 | −14.9 | −65.2 | 54.7 | 33 | A |
| GLN | CD | 113 | −14.6 | −65.0 | 53.2 | 35 | A |
| GLN | OE1 | 113 | −13.7 | −65.7 | 52.7 | 33 | A |
| GLN | NE2 | 113 | −15.4 | −64.2 | 52.5 | 33 | A |
| GLN | C | 113 | −16.3 | −68.9 | 54.9 | 26 | A |
| GLN | O | 113 | −15.8 | −69.3 | 56.0 | 29 | A |
| GLY | N | 114 | −16.7 | −69.8 | 54.0 | 27 | A |
| GLY | CA | 114 | −16.7 | −71.2 | 54.2 | 23 | A |
| GLY | C | 114 | −18.0 | −71.9 | 54.0 | 23 | A |
| GLY | O | 114 | −19.1 | −71.4 | 54.1 | 18 | A |
| LYS | N | 115 | −17.8 | −73.2 | 53.7 | 24 | A |
| LYS | CA | 115 | −19.0 | −74.1 | 53.5 | 26 | A |
| LYS | CB | 115 | −19.8 | −73.7 | 52.2 | 27 | A |
| LYS | CG | 115 | −18.9 | −73.8 | 50.9 | 32 | A |
| LYS | CD | 115 | −19.8 | −73.8 | 49.7 | 33 | A |
| LYS | CE | 115 | −20.7 | −72.6 | 49.5 | 35 | A |
| LYS | NZ | 115 | −21.5 | −72.8 | 48.2 | 35 | A |
| LYS | C | 115 | −18.5 | −75.6 | 53.4 | 23 | A |
| LYS | O | 115 | −17.5 | −75.9 | 52.8 | 22 | A |
| TRP | N | 116 | −19.3 | −76.5 | 54.0 | 21 | A |
| TRP | CA | 116 | −19.0 | −77.9 | 53.9 | 17 | A |
| TRP | CB | 116 | −18.1 | −78.3 | 55.1 | 16 | A |
| TRP | CG | 116 | −18.6 | −77.9 | 56.4 | 15 | A |
| TRP | CD2 | 116 | −19.6 | −78.4 | 57.2 | 14 | A |
| TRP | CE2 | 116 | −19.7 | −77.7 | 58.4 | 14 | A |
| TRP | CE3 | 116 | −20.6 | −79.4 | 57.0 | 11 | A |
| TRP | CD1 | 116 | −18.0 | −76.9 | 57.2 | 15 | A |
| TRP | NE1 | 116 | −18.6 | −76.8 | 58.4 | 16 | A |
| TRP | CZ2 | 116 | −20.6 | −78.0 | 59.4 | 10 | A |
| TRP | CZ3 | 116 | −21.5 | −79.7 | 58.0 | 9 | A |
| TRP | CH2 | 116 | −21.5 | −79.0 | 59.2 | 7 | A |
| TRP | C | 116 | −20.3 | −78.7 | 54.0 | 17 | A |
| TRP | O | 116 | −21.3 | −78.3 | 54.5 | 16 | A |
| GLU | N | 117 | −20.2 | −79.9 | 53.3 | 17 | A |
| GLU | CA | 117 | −21.4 | −80.8 | 53.2 | 18 | A |
| GLU | CB | 117 | −21.7 | −81.1 | 51.7 | 23 | A |
| GLU | CG | 117 | −21.9 | −82.6 | 51.3 | 31 | A |
| GLU | CD | 117 | −23.3 | −83.1 | 51.7 | 39 | A |
| GLU | OE1 | 117 | −24.3 | −82.5 | 51.3 | 42 | A |
| GLU | OE2 | 117 | −23.3 | −84.2 | 52.3 | 43 | A |
| GLU | C | 117 | −20.9 | −82.1 | 53.9 | 17 | A |
| GLU | O | 117 | −19.8 | −82.6 | 53.6 | 12 | A |
| GLY | N | 118 | −21.7 | −82.7 | 54.7 | 16 | A |
| GLY | CA | 118 | −21.3 | −83.9 | 55.4 | 18 | A |
| GLY | C | 118 | −22.4 | −85.0 | 55.7 | 19 | A |
| GLY | O | 118 | −23.5 | −84.8 | 55.3 | 20 | A |
| GLU | N | 119 | −22.0 | −86.0 | 56.5 | 21 | A |
| GLU | CA | 119 | −23.0 | −87.1 | 56.8 | 22 | A |
| GLU | CB | 119 | −22.4 | −88.4 | 56.3 | 27 | A |
| GLU | CG | 119 | −22.3 | −88.4 | 54.7 | 34 | A |
| GLU | CD | 119 | −21.5 | −89.6 | 54.2 | 36 | A |
| GLU | OE1 | 119 | −21.4 | −89.7 | 52.9 | 36 | A |
| GLU | OE2 | 119 | −20.9 | −90.4 | 55.0 | 35 | A |
| GLU | C | 119 | −23.2 | −87.1 | 58.3 | 19 | A |
| GLU | O | 119 | −22.3 | −87.4 | 59.1 | 19 | A |
| LEU | N | 120 | −24.5 | −86.9 | 58.7 | 15 | A |
| LEU | CA | 120 | −24.9 | −86.9 | 60.1 | 16 | A |
| LEU | CB | 120 | −26.3 | −86.3 | 60.3 | 14 | A |
| LEU | CG | 120 | −26.4 | −84.8 | 60.0 | 19 | A |
| LEU | CD1 | 120 | −27.9 | −84.4 | 60.1 | 19 | A |
| LEU | CD2 | 120 | −25.6 | −84.0 | 61.0 | 19 | A |
| LEU | C | 120 | −24.9 | −88.2 | 60.8 | 17 | A |
| LEU | O | 120 | −25.4 | −89.2 | 60.3 | 20 | A |
| GLY | N | 121 | −24.2 | −88.3 | 62.0 | 18 | A |
| GLY | CA | 121 | −24.1 | −89.5 | 62.8 | 18 | A |
| GLY | C | 121 | −24.1 | −89.1 | 64.2 | 20 | A |
| GLY | O | 121 | −24.4 | −87.9 | 64.6 | 23 | A |
| THR | N | 122 | −23.7 | −90.0 | 65.1 | 20 | A |
| THR | CA | 122 | −23.5 | −89.7 | 66.5 | 20 | A |
| THR | CB | 122 | −24.8 | −90.0 | 67.3 | 18 | A |
| THR | OG1 | 122 | −24.8 | −91.4 | 67.6 | 15 | A |
| THR | CG2 | 122 | −26.0 | −89.5 | 66.6 | 15 | A |
| THR | C | 122 | −22.3 | −90.5 | 67.1 | 20 | A |
| THR | O | 122 | −21.8 | −91.3 | 66.4 | 20 | A |
| ASP | N | 123 | −21.9 | −90.2 | 68.3 | 20 | A |
| ASP | CA | 123 | −20.8 | −90.9 | 68.9 | 17 | A |
| ASP | CB | 123 | −19.6 | −90.7 | 68.0 | 12 | A |
| ASP | CG | 123 | −18.7 | −92.0 | 68.1 | 11 | A |
| ASP | OD1 | 123 | −18.4 | −92.4 | 69.2 | 11 | A |
| ASP | OD2 | 123 | −18.3 | −92.5 | 67.0 | 15 | A |
| ASP | C | 123 | −20.5 | −90.3 | 70.3 | 17 | A |
| ASP | O | 123 | −21.1 | −89.3 | 70.8 | 16 | A |
| LEU | N | 124 | −19.5 | −90.9 | 71.0 | 16 | A |
| LEU | CA | 124 | −19.1 | −90.4 | 72.3 | 16 | A |
| LEU | CB | 124 | −18.6 | −91.6 | 73.1 | 18 | A |
| LEU | CG | 124 | −19.5 | −92.7 | 73.4 | 19 | A |
| LEU | CD1 | 124 | −18.9 | −93.8 | 74.2 | 18 | A |
| LEU | CD2 | 124 | −20.7 | −92.2 | 74.1 | 21 | A |
| LEU | C | 124 | −18.0 | −89.3 | 72.2 | 16 | A |
| LEU | O | 124 | −17.0 | −89.5 | 71.5 | 17 | A |
| VAL | N | 125 | −18.3 | −88.2 | 72.8 | 12 | A |
| VAL | CA | 125 | −17.4 | −87.1 | 72.8 | 11 | A |
| VAL | CB | 125 | −18.0 | −85.9 | 72.2 | 12 | A |
| VAL | CG1 | 125 | −17.0 | −84.7 | 72.2 | 13 | A |
| VAL | CG2 | 125 | −18.5 | −86.1 | 70.8 | 12 | A |
| VAL | C | 125 | −16.8 | −86.8 | 74.1 | 10 | A |
| VAL | O | 125 | −17.4 | −87.0 | 75.1 | 5 | A |
| SER | N | 126 | −15.5 | −86.4 | 74.1 | 11 | A |
| SER | CA | 126 | −14.8 | −86.0 | 75.3 | 12 | A |
| SER | CB | 126 | −13.9 | −87.2 | 75.8 | 14 | A |
| SER | OG | 126 | −14.6 | −88.1 | 76.6 | 12 | A |
| SER | C | 126 | −13.9 | −84.8 | 75.1 | 12 | A |
| SER | O | 126 | −13.5 | −84.5 | 74.0 | 12 | A |
| ILE | N | 127 | −13.6 | −84.2 | 76.2 | 12 | A |
| ILE | CA | 127 | −12.8 | −83.0 | 76.2 | 13 | A |
| ILE | CB | 127 | −13.6 | −81.7 | 76.6 | 9 | A |
| ILE | CG2 | 127 | −12.6 | −80.5 | 76.8 | 12 | A |
| ILE | CG1 | 127 | −14.6 | −81.4 | 75.5 | 10 | A |
| ILE | CD1 | 127 | −15.5 | −80.2 | 75.8 | 12 | A |
| ILE | C | 127 | −11.7 | −83.2 | 77.3 | 12 | A |
| ILE | O | 127 | −11.9 | −83.0 | 78.4 | 15 | A |
| PRO | N | 128 | −10.5 | −83.7 | 76.8 | 11 | A |
| PRO | CD | 128 | −10.2 | −83.9 | 75.4 | 11 | A |
| PRO | CA | 128 | −9.3 | −84.0 | 77.6 | 10 | A |
| PRO | CB | 128 | −8.2 | −84.1 | 76.6 | 13 | A |
| PRO | CG | 128 | −8.9 | −84.7 | 75.4 | 10 | A |
| PRO | C | 128 | −9.1 | −83.0 | 78.7 | 10 | A |
| PRO | O | 128 | −9.0 | −83.3 | 79.9 | 11 | A |
| HIS | N | 129 | −8.9 | −81.7 | 78.3 | 8 | A |
| HIS | CA | 129 | −8.6 | −80.7 | 79.3 | 7 | A |
| HIS | CB | 129 | −7.6 | −79.7 | 78.8 | 7 | A |
| HIS | CG | 129 | −6.3 | −80.5 | 78.3 | 8 | A |
| HIS | CD2 | 129 | −5.7 | −80.5 | 77.2 | 9 | A |
| HIS | ND1 | 129 | −5.7 | −81.4 | 79.2 | 7 | A |
| HIS | CE1 | 129 | −4.6 | −81.9 | 78.5 | 11 | A |
| HIS | NE2 | 129 | −4.6 | −81.3 | 77.3 | 6 | A |
| HIS | C | 129 | −9.9 | −79.9 | 79.6 | 6 | A |
| HIS | O | 129 | −10.0 | −78.7 | 79.5 | 6 | A |
| GLY | N | 130 | −10.9 | −80.6 | 80.0 | 5 | A |
| GLY | CA | 130 | −12.2 | −80.0 | 80.4 | 5 | A |
| GLY | C | 130 | −12.7 | −80.9 | 81.4 | 6 | A |
| GLY | O | 130 | −12.0 | −81.7 | 82.1 | 5 | A |
| PRO | N | 131 | −14.1 | −80.9 | 81.7 | 8 | A |
| PRO | CD | 131 | −15.1 | −80.0 | 81.1 | 6 | A |
| PRO | CA | 131 | −14.7 | −81.7 | 82.7 | 12 | A |
| PRO | CB | 131 | −16.1 | −81.2 | 82.8 | 12 | A |
| PRO | CG | 131 | −16.3 | −80.7 | 81.4 | 8 | A |
| PRO | C | 131 | −14.5 | −83.2 | 82.4 | 16 | A |
| PRO | O | 131 | −14.7 | −83.6 | 81.3 | 15 | A |
| ASN | N | 132 | −14.2 | −84.0 | 83.4 | 20 | A |
| ASN | CA | 132 | −14.0 | −85.4 | 83.3 | 24 | A |
| ASN | CB | 132 | −13.4 | −86.1 | 84.5 | 28 | A |
| ASN | CG | 132 | −12.9 | −87.5 | 84.3 | 32 | A |
| ASN | OD1 | 132 | −12.3 | −88.1 | 85.2 | 34 | A |
| ASN | ND2 | 132 | −13.3 | −88.1 | 83.2 | 34 | A |
| ASN | C | 132 | −15.3 | −86.1 | 82.9 | 24 | A |
| ASN | O | 132 | −15.9 | −86.8 | 83.8 | 25 | A |
| VAL | N | 133 | −15.8 | −86.0 | 81.7 | 22 | A |
| VAL | CA | 133 | −17.0 | −86.6 | 81.3 | 19 | A |
| VAL | CB | 133 | −18.2 | −85.7 | 81.5 | 16 | A |
| VAL | CG1 | 133 | −18.4 | −85.3 | 82.9 | 13 | A |
| VAL | CG2 | 133 | −18.1 | −84.4 | 80.6 | 11 | A |
| VAL | C | 133 | −17.0 | −87.0 | 79.8 | 19 | A |
| VAL | O | 133 | −16.1 | −86.6 | 79.1 | 17 | A |
| THR | N | 134 | −18.0 | −87.8 | 79.4 | 20 | A |
| THR | CA | 134 | −18.2 | −88.3 | 78.0 | 20 | A |
| THR | CB | 134 | −17.6 | −89.7 | 77.8 | 21 | A |
| THR | OG1 | 134 | −16.2 | −89.7 | 77.9 | 18 | A |
| THR | CG2 | 134 | −18.1 | −90.3 | 76.5 | 17 | A |
| THR | C | 134 | −19.7 | −88.2 | 77.8 | 18 | A |
| THR | O | 134 | −20.5 | −88.8 | 78.5 | 14 | A |
| VAL | N | 135 | −20.0 | −87.6 | 76.6 | 18 | A |
| VAL | CA | 135 | −21.4 | −87.5 | 76.2 | 18 | A |
| VAL | CB | 135 | −22.0 | −86.1 | 76.2 | 16 | A |
| VAL | CG1 | 135 | −21.9 | −85.5 | 77.7 | 19 | A |
| VAL | CG2 | 135 | −21.1 | −85.2 | 75.3 | 18 | A |
| VAL | C | 135 | −21.6 | −88.1 | 74.8 | 17 | A |
| VAL | O | 135 | −20.6 | −88.1 | 74.0 | 19 | A |
| ARG | N | 136 | −22.8 | −88.5 | 74.5 | 14 | A |
| ARG | CA | 136 | −23.1 | −88.9 | 73.1 | 13 | A |
| ARG | CB | 136 | −24.1 | −90.0 | 73.1 | 15 | A |
| ARG | CG | 136 | −24.5 | −90.5 | 71.7 | 19 | A |
| ARG | CD | 136 | −25.2 | −91.8 | 71.7 | 21 | A |
| ARG | NE | 136 | −24.4 | −92.9 | 72.2 | 25 | A |
| ARG | CZ | 136 | −23.5 | −93.5 | 71.5 | 25 | A |
| ARG | NH1 | 136 | −23.2 | −93.1 | 70.2 | 19 | A |
| ARG | NH2 | 136 | −22.8 | −94.6 | 72.0 | 23 | A |
| ARG | C | 136 | −23.7 | −87.7 | 72.5 | 12 | A |
| ARG | O | 136 | −24.7 | −87.2 | 72.9 | 12 | A |
| ALA | N | 137 | −23.0 | −87.2 | 71.4 | 11 | A |
| ALA | CA | 137 | −23.5 | −86.0 | 70.8 | 11 | A |
| ALA | CB | 137 | −22.6 | −84.8 | 71.0 | 11 | A |
| ALA | C | 137 | −23.6 | −86.2 | 69.3 | 13 | A |
| ALA | O | 137 | −23.2 | −87.2 | 68.8 | 13 | A |
| ASN | N | 138 | −24.2 | −85.2 | 68.6 | 13 | A |
| ASN | CA | 138 | −24.4 | −85.3 | 67.2 | 13 | A |
| ASN | CB | 138 | −25.5 | −84.4 | 66.7 | 13 | A |
| ASN | CG | 138 | −26.9 | −84.9 | 67.2 | 15 | A |
| ASN | OD1 | 138 | −27.2 | −86.1 | 67.1 | 13 | A |
| ASN | ND2 | 138 | −27.7 | −84.0 | 67.6 | 14 | A |
| ASN | C | 138 | −23.1 | −84.8 | 66.5 | 12 | A |
| ASN | O | 138 | −22.5 | −83.8 | 67.0 | 14 | A |
| ILE | N | 139 | −22.6 | −85.5 | 65.5 | 11 | A |
| ILE | CA | 139 | −21.4 | −85.2 | 64.9 | 13 | A |
| ILE | CB | 139 | −20.2 | −86.1 | 65.3 | 12 | A |
| ILE | CG2 | 139 | −18.9 | −85.5 | 64.7 | 12 | A |
| ILE | CG1 | 139 | −20.1 | −86.2 | 66.8 | 11 | A |
| ILE | CD1 | 139 | −18.9 | −86.9 | 67.3 | 14 | A |
| ILE | C | 139 | −21.5 | −85.3 | 63.4 | 15 | A |
| ILE | O | 139 | −21.8 | −86.3 | 62.8 | 16 | A |
| ALA | N | 140 | −21.4 | −84.1 | 62.7 | 17 | A |
| ALA | CA | 140 | −21.5 | −84.1 | 61.3 | 14 | A |
| ALA | CB | 140 | −21.9 | −82.7 | 60.8 | 12 | A |
| ALA | C | 140 | −20.1 | −84.5 | 60.7 | 15 | A |
| ALA | O | 140 | −19.1 | −83.8 | 60.9 | 15 | A |
| ALA | N | 141 | −20.1 | −85.6 | 60.0 | 15 | A |
| ALA | CA | 141 | −18.9 | −86.1 | 59.4 | 15 | A |
| ALA | CB | 141 | −19.0 | −87.6 | 59.2 | 15 | A |
| ALA | C | 141 | −18.7 | −85.4 | 58.1 | 14 | A |
| ALA | O | 141 | −19.4 | −85.7 | 57.1 | 11 | A |
| ILE | N | 142 | −17.8 | −84.4 | 58.0 | 14 | A |
| ILE | CA | 142 | −17.6 | −83.6 | 56.8 | 14 | A |
| ILE | CB | 142 | −16.7 | −82.4 | 57.2 | 15 | A |
| ILE | CG2 | 142 | −16.3 | −81.7 | 55.9 | 15 | A |
| ILE | CG1 | 142 | −17.5 | −81.4 | 58.0 | 12 | A |
| ILE | CD1 | 142 | −16.8 | −80.1 | 58.4 | 12 | A |
| ILE | C | 142 | −16.9 | −84.5 | 55.7 | 13 | A |
| ILE | O | 142 | −15.8 | −84.9 | 55.8 | 9 | A |
| THR | N | 143 | −17.7 | −84.6 | 54.7 | 14 | A |
| THR | CA | 143 | −17.3 | −85.4 | 53.5 | 16 | A |
| THR | CB | 143 | −18.4 | −86.3 | 53.0 | 20 | A |
| THR | OG1 | 143 | −19.6 | −85.5 | 52.9 | 19 | A |
| THR | CG2 | 143 | −18.7 | −87.5 | 53.9 | 18 | A |
| THR | C | 143 | −16.8 | −84.6 | 52.4 | 15 | A |
| THR | O | 143 | −16.0 | −85.0 | 51.6 | 13 | A |
| GLU | N | 144 | −17.3 | −83.4 | 52.3 | 18 | A |
| GLU | CA | 144 | −16.9 | −82.4 | 51.3 | 25 | A |
| GLU | CB | 144 | −18.0 | −82.4 | 50.2 | 29 | A |
| GLU | CG | 144 | −17.6 | −81.6 | 48.9 | 34 | A |
| GLU | CD | 144 | −18.6 | −81.9 | 47.8 | 40 | A |
| GLU | OE1 | 144 | −18.6 | −83.0 | 47.3 | 40 | A |
| GLU | OE2 | 144 | −19.3 | −80.9 | 47.4 | 41 | A |
| GLU | C | 144 | −16.7 | −81.1 | 51.9 | 28 | A |
| GLU | O | 144 | −17.5 | −80.6 | 52.8 | 29 | A |
| SER | N | 145 | −15.6 | −80.4 | 51.5 | 30 | A |
| SER | CA | 145 | −15.3 | −79.1 | 52.1 | 28 | A |
| SER | CB | 145 | −14.3 | −79.3 | 53.2 | 28 | A |
| SER | OG | 145 | −13.4 | −80.4 | 52.9 | 26 | A |
| SER | C | 145 | −14.7 | −78.1 | 51.1 | 29 | A |
| SER | O | 145 | −14.1 | −78.5 | 50.1 | 29 | A |
| ASP | N | 146 | −15.0 | −76.8 | 51.3 | 27 | A |
| ASP | CA | 146 | −14.6 | −75.8 | 50.4 | 26 | A |
| ASP | CB | 146 | −15.5 | −75.7 | 49.2 | 24 | A |
| ASP | CG | 146 | −15.2 | −74.6 | 48.3 | 25 | A |
| ASP | OD1 | 146 | −14.1 | −74.5 | 47.9 | 26 | A |
| ASP | OD2 | 146 | −16.2 | −73.8 | 47.9 | 27 | A |
| ASP | C | 146 | −14.5 | −74.4 | 51.1 | 26 | A |
| ASP | O | 146 | −15.5 | −73.9 | 51.6 | 25 | A |
| LYS | N | 147 | −13.3 | −73.8 | 51.2 | 24 | A |
| LYS | CA | 147 | −13.1 | −72.5 | 51.8 | 23 | A |
| LYS | CB | 147 | −14.0 | −71.5 | 51.2 | 26 | A |
| LYS | CG | 147 | −13.9 | −71.2 | 49.7 | 22 | A |
| LYS | CD | 147 | −12.7 | −70.3 | 49.4 | 25 | A |
| LYS | CE | 147 | −12.7 | −69.9 | 48.0 | 24 | A |
| LYS | NZ | 147 | −11.5 | −69.2 | 47.6 | 27 | A |
| LYS | C | 147 | −13.3 | −72.6 | 53.3 | 22 | A |
| LYS | O | 147 | −13.5 | −71.6 | 54.0 | 18 | A |
| PHE | N | 148 | −13.2 | −73.8 | 53.9 | 21 | A |
| PHE | CA | 148 | −13.4 | −74.0 | 55.3 | 22 | A |
| PHE | CB | 148 | −14.3 | −75.2 | 55.6 | 17 | A |
| PHE | CG | 148 | −14.8 | −75.3 | 57.0 | 14 | A |
| PHE | CD1 | 148 | −15.8 | −74.4 | 57.5 | 16 | A |
| PHE | CD2 | 148 | −14.3 | −76.3 | 57.8 | 15 | A |
| PHE | CE1 | 148 | −16.3 | −74.6 | 58.8 | 16 | A |
| PHE | CE2 | 148 | −14.8 | −76.5 | 59.1 | 14 | A |
| PHE | CZ | 148 | −15.8 | −75.6 | 59.6 | 12 | A |
| PHE | C | 148 | −12.0 | −74.3 | 56.0 | 23 | A |
| PHE | O | 148 | −11.4 | −73.3 | 56.6 | 25 | A |
| PHE | N | 149 | −11.6 | −75.5 | 55.9 | 21 | A |
| PHE | CA | 149 | −10.3 | −75.9 | 56.4 | 18 | A |
| PHE | CB | 149 | −10.0 | −77.4 | 56.1 | 16 | A |
| PHE | CG | 149 | −11.0 | −78.3 | 56.7 | 16 | A |
| PHE | CD1 | 149 | −11.5 | −79.3 | 56.0 | 12 | A |
| PHE | CD2 | 149 | −11.5 | −78.1 | 58.0 | 15 | A |
| PHE | CE1 | 149 | −12.5 | −80.2 | 56.5 | 15 | A |
| PHE | CE2 | 149 | −12.4 | −79.0 | 58.6 | 13 | A |
| PHE | CZ | 149 | −12.9 | −80.0 | 57.8 | 12 | A |
| PHE | C | 149 | −9.1 | −75.0 | 55.9 | 17 | A |
| PHE | O | 149 | −9.0 | −75.0 | 54.7 | 19 | A |
| ILE | N | 150 | −8.4 | −74.4 | 56.8 | 16 | A |
| ILE | CA | 150 | −7.3 | −73.5 | 56.4 | 14 | A |
| ILE | CB | 150 | −7.2 | −72.3 | 57.4 | 9 | A |
| ILE | CG2 | 150 | −6.1 | −71.3 | 56.9 | 9 | A |
| ILE | CG1 | 150 | −8.6 | −71.7 | 57.4 | 6 | A |
| ILE | CD1 | 150 | −8.7 | −70.5 | 58.4 | 6 | A |
| ILE | C | 150 | −6.0 | −74.2 | 56.3 | 18 | A |
| ILE | O | 150 | −5.7 | −75.1 | 57.2 | 19 | A |
| ASN | N | 151 | −5.1 | −73.9 | 55.4 | 23 | A |
| ASN | CA | 151 | −3.8 | −74.5 | 55.3 | 26 | A |
| ASN | CB | 151 | −3.0 | −74.1 | 54.1 | 28 | A |
| ASN | CG | 151 | −1.8 | −75.0 | 53.8 | 34 | A |
| ASN | OD1 | 151 | −1.1 | −74.9 | 52.7 | 36 | A |
| ASN | ND2 | 151 | −1.6 | −76.0 | 54.7 | 33 | A |
| ASN | C | 151 | −2.9 | −74.3 | 56.5 | 26 | A |
| ASN | O | 151 | −2.5 | −73.2 | 56.8 | 25 | A |
| GLY | N | 152 | −2.7 | −75.4 | 57.3 | 26 | A |
| GLY | CA | 152 | −1.9 | −75.3 | 58.5 | 28 | A |
| GLY | C | 152 | −2.6 | −74.6 | 59.7 | 29 | A |
| GLY | O | 152 | −1.9 | −74.0 | 60.5 | 28 | A |
| SER | N | 153 | −3.9 | −74.6 | 59.7 | 28 | A |
| SER | CA | 153 | −4.7 | −73.9 | 60.8 | 27 | A |
| SER | CB | 153 | −6.1 | −73.9 | 60.5 | 28 | A |
| SER | OG | 153 | −6.7 | −75.2 | 60.5 | 27 | A |
| SER | C | 153 | −4.5 | −74.7 | 62.1 | 23 | A |
| SER | O | 153 | −4.5 | −74.2 | 63.2 | 22 | A |
| ASN | N | 154 | −4.3 | −76.0 | 61.9 | 21 | A |
| ASN | CA | 154 | −4.1 | −77.0 | 62.9 | 18 | A |
| ASN | CB | 154 | −3.1 | −76.5 | 64.0 | 20 | A |
| ASN | CG | 154 | −2.4 | −77.6 | 64.7 | 23 | A |
| ASN | OD1 | 154 | −1.9 | −78.6 | 64.1 | 23 | A |
| ASN | ND2 | 154 | −2.3 | −77.5 | 66.0 | 26 | A |
| ASN | C | 154 | −5.3 | −77.5 | 63.6 | 15 | A |
| ASN | O | 154 | −5.3 | −78.2 | 64.6 | 15 | A |
| TRP | N | 155 | −6.5 | −77.1 | 63.1 | 13 | A |
| TRP | CA | 155 | −7.7 | −77.6 | 63.6 | 12 | A |
| TRP | CB | 155 | −8.6 | −76.5 | 64.2 | 12 | A |
| TRP | CG | 155 | −8.9 | −75.3 | 63.3 | 11 | A |
| TRP | CD2 | 155 | −9.9 | −75.2 | 62.3 | 13 | A |
| TRP | CE2 | 155 | −9.8 | −73.9 | 61.8 | 15 | A |
| TRP | CE3 | 155 | −10.9 | −76.1 | 61.8 | 12 | A |
| TRP | CD1 | 155 | −8.2 | −74.1 | 63.3 | 12 | A |
| TRP | NE1 | 155 | −8.8 | −73.3 | 62.4 | 14 | A |
| TRP | CZ2 | 155 | −10.7 | −73.4 | 60.8 | 15 | A |
| TRP | CZ3 | 155 | −11.7 | −75.6 | 60.8 | 13 | A |
| TRP | CH2 | 155 | −11.6 | −74.3 | 60.3 | 15 | A |
| TRP | C | 155 | −8.4 | −78.4 | 62.5 | 13 | A |
| TRP | O | 155 | −8.3 | −78.0 | 61.4 | 15 | A |
| GLU | N | 156 | −9.1 | −79.5 | 62.9 | 15 | A |
| GLU | CA | 156 | −9.7 | −80.3 | 61.9 | 15 | A |
| GLU | CB | 156 | −9.1 | −81.8 | 61.9 | 19 | A |
| GLU | CG | 156 | −7.6 | −81.8 | 61.8 | 26 | A |
| GLU | CD | 156 | −6.9 | −81.7 | 63.2 | 30 | A |
| GLU | OE1 | 156 | −5.7 | −81.6 | 63.3 | 32 | A |
| GLU | OE2 | 156 | −7.7 | −81.7 | 64.2 | 35 | A |
| GLU | C | 156 | −11.2 | −80.4 | 62.1 | 13 | A |
| GLU | O | 156 | −11.9 | −81.3 | 61.5 | 13 | A |
| GLY | N | 157 | −11.8 | −79.5 | 62.9 | 12 | A |
| GLY | CA | 157 | −13.2 | −79.6 | 63.1 | 10 | A |
| GLY | C | 157 | −13.7 | −78.3 | 63.8 | 9 | A |
| GLY | O | 157 | −12.9 | −77.4 | 64.1 | 9 | A |
| ILE | N | 158 | −15.0 | −78.2 | 63.9 | 8 | A |
| ILE | CA | 158 | −15.6 | −77.0 | 64.5 | 7 | A |
| ILE | CB | 158 | −16.3 | −76.1 | 63.4 | 7 | A |
| ILE | CG2 | 158 | −17.3 | −76.9 | 62.7 | 8 | A |
| ILE | CG1 | 158 | −16.9 | −74.9 | 64.0 | 11 | A |
| ILE | CD1 | 158 | −17.6 | −74.0 | 63.0 | 16 | A |
| ILE | C | 158 | −16.7 | −77.4 | 65.5 | 8 | A |
| ILE | O | 158 | −17.4 | −78.3 | 65.4 | 7 | A |
| LEU | N | 159 | −16.7 | −76.6 | 66.6 | 7 | A |
| LEU | CA | 159 | −17.7 | −76.8 | 67.7 | 5 | A |
| LEU | CB | 159 | −16.9 | −77.1 | 69.1 | 1 | A |
| LEU | CG | 159 | −17.7 | −77.2 | 70.4 | 1 | A |
| LEU | CD1 | 159 | −18.4 | −78.6 | 70.4 | 1 | A |
| LEU | CD2 | 159 | −16.8 | −77.2 | 71.5 | 1 | A |
| LEU | C | 159 | −18.6 | −75.6 | 67.9 | 7 | A |
| LEU | O | 159 | −18.2 | −74.6 | 68.6 | 5 | A |
| GLY | N | 160 | −19.8 | −75.7 | 67.3 | 6 | A |
| GLY | CA | 160 | −20.7 | −74.6 | 67.5 | 9 | A |
| GLY | C | 160 | −21.3 | −74.5 | 68.9 | 9 | A |
| GLY | O | 160 | −22.2 | −75.4 | 69.2 | 12 | A |
| LEU | N | 161 | −21.0 | −73.5 | 69.7 | 8 | A |
| LEU | CA | 161 | −21.5 | −73.4 | 71.0 | 6 | A |
| LEU | CB | 161 | −20.4 | −72.8 | 71.9 | 3 | A |
| LEU | CG | 161 | −19.2 | −73.8 | 72.1 | 3 | A |
| LEU | CD1 | 161 | −18.1 | −73.1 | 72.8 | 3 | A |
| LEU | CD2 | 161 | −19.6 | −75.0 | 72.9 | 3 | A |
| LEU | C | 161 | −22.7 | −72.6 | 71.1 | 6 | A |
| LEU | O | 161 | −23.2 | −72.3 | 72.2 | 7 | A |
| ALA | N | 162 | −23.3 | −72.2 | 69.9 | 5 | A |
| ALA | CA | 162 | −24.5 | −71.4 | 69.9 | 6 | A |
| ALA | CB | 162 | −24.7 | −70.8 | 68.5 | 4 | A |
| ALA | C | 162 | −25.7 | −72.2 | 70.3 | 4 | A |
| ALA | O | 162 | −25.5 | −73.3 | 70.8 | 4 | A |
| TYR | N | 163 | −26.9 | −71.7 | 70.0 | 4 | A |
| TYR | CA | 163 | −28.1 | −72.4 | 70.3 | 5 | A |
| TYR | CB | 163 | −29.1 | −71.4 | 70.8 | 7 | A |
| TYR | CG | 163 | −28.8 | −70.7 | 72.1 | 7 | A |
| TYR | CD1 | 163 | −27.9 | −69.6 | 72.1 | 10 | A |
| TYR | CE1 | 163 | −27.6 | −69.0 | 73.3 | 11 | A |
| TYR | CD2 | 163 | −29.4 | −71.1 | 73.3 | 8 | A |
| TYR | CE2 | 163 | −29.1 | −70.4 | 74.5 | 10 | A |
| TYR | CZ | 163 | −28.2 | −69.4 | 74.5 | 8 | A |
| TYR | OH | 163 | −27.9 | −68.7 | 75.7 | 5 | A |
| TYR | C | 163 | −28.6 | −73.2 | 69.1 | 6 | A |
| TYR | O | 163 | −28.3 | −72.9 | 68.0 | 1 | A |
| ALA | N | 164 | −29.5 | −74.1 | 69.4 | 8 | A |
| ALA | CA | 164 | −30.1 | −75.0 | 68.3 | 8 | A |
| ALA | CB | 164 | −30.9 | −76.1 | 69.0 | 7 | A |
| ALA | C | 164 | −30.9 | −74.3 | 67.3 | 6 | A |
| ALA | O | 164 | −30.9 | −74.7 | 66.1 | 3 | A |
| GLU | N | 165 | −31.5 | −73.2 | 67.7 | 8 | A |
| GLU | CA | 165 | −32.3 | −72.4 | 66.8 | 9 | A |
| GLU | CB | 165 | −32.7 | −71.0 | 67.4 | 13 | A |
| GLU | CG | 165 | −33.3 | −70.0 | 66.5 | 18 | A |
| GLU | CD | 165 | −34.8 | −70.1 | 66.5 | 25 | A |
| GLU | OE1 | 165 | −35.4 | −69.7 | 67.6 | 28 | A |
| GLU | OE2 | 165 | −35.5 | −70.5 | 65.5 | 28 | A |
| GLU | C | 165 | −31.6 | −72.1 | 65.4 | 6 | A |
| GLU | O | 165 | −32.3 | −72.0 | 64.4 | 5 | A |
| ILE | N | 166 | −30.3 | −72.1 | 65.3 | 5 | A |
| ILE | CA | 166 | −29.6 | −71.8 | 64.1 | 5 | A |
| ILE | CB | 166 | −28.7 | −70.6 | 64.1 | 4 | A |
| ILE | CG2 | 166 | −29.4 | −69.4 | 64.6 | 6 | A |
| ILE | CG1 | 166 | −27.5 | −71.0 | 65.0 | 3 | A |
| ILE | CD1 | 166 | −26.4 | −69.9 | 65.1 | 1 | A |
| ILE | C | 166 | −28.8 | −73.0 | 63.6 | 10 | A |
| ILE | O | 166 | −28.0 | −72.9 | 62.7 | 11 | A |
| ALA | N | 167 | −29.1 | −74.2 | 64.2 | 12 | A |
| ALA | CA | 167 | −28.4 | −75.4 | 63.8 | 15 | A |
| ALA | CB | 167 | −28.4 | −76.4 | 65.0 | 15 | A |
| ALA | C | 167 | −29.0 | −76.0 | 62.6 | 16 | A |
| ALA | O | 167 | −30.2 | −76.1 | 62.4 | 16 | A |
| ARG | N | 168 | −28.1 | −76.4 | 61.6 | 14 | A |
| ARG | CA | 168 | −28.5 | −77.1 | 60.4 | 12 | A |
| ARG | CB | 168 | −27.4 | −76.8 | 59.3 | 14 | A |
| ARG | CG | 168 | −27.3 | −75.4 | 59.0 | 16 | A |
| ARG | CD | 168 | −28.2 | −75.0 | 57.9 | 20 | A |
| ARG | NE | 168 | −28.0 | −73.6 | 57.4 | 30 | A |
| ARG | CZ | 168 | −28.4 | −73.1 | 56.2 | 36 | A |
| ARG | NH1 | 168 | −29.0 | −74.0 | 55.4 | 35 | A |
| ARG | NH2 | 168 | −28.1 | −71.9 | 55.9 | 33 | A |
| ARG | C | 168 | −28.6 | −78.6 | 60.7 | 10 | A |
| ARG | O | 168 | −27.8 | −79.1 | 61.5 | 8 | A |
| PRO | N | 169 | −29.5 | −79.3 | 60.1 | 9 | A |
| PRO | CD | 169 | −29.6 | −80.8 | 60.4 | 11 | A |
| PRO | CA | 169 | −30.6 | −79.0 | 59.1 | 9 | A |
| PRO | CB | 169 | −31.1 | −80.3 | 58.7 | 9 | A |
| PRO | CG | 169 | −30.0 | −81.3 | 59.0 | 12 | A |
| PRO | C | 169 | −31.6 | −78.1 | 59.8 | 10 | A |
| PRO | O | 169 | −32.1 | −77.2 | 59.2 | 15 | A |
| ASP | N | 170 | −32.1 | −78.5 | 61.0 | 10 | A |
| ASP | CA | 170 | −33.1 | −77.8 | 61.7 | 14 | A |
| ASP | CB | 170 | −34.5 | −78.4 | 61.4 | 19 | A |
| ASP | CG | 170 | −34.6 | −79.8 | 61.8 | 26 | A |
| ASP | OD1 | 170 | −34.3 | −80.7 | 61.0 | 27 | A |
| ASP | OD2 | 170 | −35.1 | −80.0 | 63.0 | 29 | A |
| ASP | C | 170 | −32.8 | −77.8 | 63.2 | 14 | A |
| ASP | O | 170 | −32.0 | −78.5 | 63.6 | 11 | A |
| ASP | N | 171 | −33.6 | −77.0 | 63.9 | 13 | A |
| ASP | CA | 171 | −33.5 | −76.8 | 65.3 | 13 | A |
| ASP | CB | 171 | −34.4 | −75.7 | 65.8 | 16 | A |
| ASP | CG | 171 | −35.9 | −76.0 | 65.6 | 16 | A |
| ASP | OD1 | 171 | −36.1 | −77.1 | 65.0 | 14 | A |
| ASP | OD2 | 171 | −36.7 | −75.2 | 65.9 | 16 | A |
| ASP | C | 171 | −33.7 | −78.1 | 66.1 | 14 | A |
| ASP | O | 171 | −34.1 | −78.0 | 67.3 | 16 | A |
| SER | N | 172 | −33.6 | −79.2 | 65.5 | 12 | A |
| SER | CA | 172 | −33.8 | −80.5 | 66.2 | 13 | A |
| SER | CB | 172 | −34.8 | −81.4 | 65.5 | 12 | A |
| SER | OG | 172 | −34.1 | −82.0 | 64.4 | 16 | A |
| SER | C | 172 | −32.5 | −81.2 | 66.5 | 14 | A |
| SER | O | 172 | −32.4 | −82.2 | 67.3 | 20 | A |
| LEU | N | 173 | −31.4 | −80.8 | 65.8 | 12 | A |
| LEU | CA | 173 | −30.1 | −81.4 | 65.9 | 10 | A |
| LEU | CB | 173 | −29.3 | −81.1 | 64.7 | 6 | A |
| LEU | CG | 173 | −28.0 | −82.0 | 64.5 | 2 | A |
| LEU | CD1 | 173 | −28.4 | −83.5 | 64.5 | 1 | A |
| LEU | CD2 | 173 | −27.4 | −81.6 | 63.1 | 2 | A |
| LEU | C | 173 | −29.4 | −80.8 | 67.2 | 11 | A |
| LEU | O | 173 | −28.6 | −79.9 | 67.0 | 9 | A |
| GLU | N | 174 | −29.8 | −81.3 | 68.3 | 13 | A |
| GLU | CA | 174 | −29.3 | −80.8 | 69.6 | 14 | A |
| GLU | CB | 174 | −29.7 | −81.8 | 70.7 | 16 | A |
| GLU | CG | 174 | −29.2 | −81.5 | 72.1 | 21 | A |
| GLU | CD | 174 | −29.7 | −82.4 | 73.2 | 25 | A |
| GLU | OE1 | 174 | −30.9 | −82.3 | 73.6 | 20 | A |
| GLU | OE2 | 174 | −28.9 | −83.3 | 73.6 | 27 | A |
| GLU | C | 174 | −27.8 | −80.7 | 69.6 | 14 | A |
| GLU | O | 174 | −27.0 | −81.6 | 69.5 | 16 | A |
| PRO | N | 175 | −27.3 | −79.4 | 69.8 | 12 | A |
| PRO | CD | 175 | −28.3 | −78.3 | 69.8 | 11 | A |
| PRO | CA | 175 | −25.9 | −78.9 | 69.8 | 10 | A |
| PRO | CB | 175 | −26.1 | −77.4 | 70.0 | 12 | A |
| PRO | CG | 175 | −27.4 | −77.1 | 69.3 | 9 | A |
| PRO | C | 175 | −25.1 | −79.5 | 70.9 | 10 | A |
| PRO | O | 175 | −25.6 | −79.7 | 72.1 | 12 | A |
| PHE | N | 176 | −23.8 | −79.8 | 70.6 | 5 | A |
| PHE | CA | 176 | −22.9 | −80.4 | 71.6 | 2 | A |
| PHE | CB | 176 | −21.4 | −80.0 | 71.3 | 4 | A |
| PHE | CG | 176 | −20.5 | −80.6 | 72.3 | 3 | A |
| PHE | CD1 | 176 | −20.1 | −81.9 | 72.2 | 3 | A |
| PHE | CD2 | 176 | −20.0 | −79.9 | 73.4 | 4 | A |
| PHE | CE1 | 176 | −19.3 | −82.5 | 73.2 | 6 | A |
| PHE | CE2 | 176 | −19.2 | −80.4 | 74.3 | 6 | A |
| PHE | CZ | 176 | −18.8 | −81.7 | 74.2 | 7 | A |
| PHE | C | 176 | −23.1 | −80.0 | 73.1 | 3 | A |
| PHE | O | 176 | −23.4 | −80.8 | 73.9 | 1 | A |
| PHE | N | 177 | −23.0 | −78.7 | 73.4 | 4 | A |
| PHE | CA | 177 | −23.2 | −78.2 | 74.7 | 5 | A |
| PHE | CB | 177 | −23.1 | −76.6 | 74.8 | 1 | A |
| PHE | CG | 177 | −22.5 | −76.1 | 76.1 | 1 | A |
| PHE | CD1 | 177 | −21.2 | −76.3 | 76.4 | 2 | A |
| PHE | CD2 | 177 | −23.3 | −75.5 | 77.0 | 1 | A |
| PHE | CE1 | 177 | −20.6 | −75.9 | 77.5 | 2 | A |
| PHE | CE2 | 177 | −22.8 | −75.0 | 78.2 | 2 | A |
| PHE | CZ | 177 | −21.4 | −75.2 | 78.5 | 3 | A |
| PHE | C | 177 | −24.5 | −78.6 | 75.3 | 7 | A |
| PHE | O | 177 | −24.6 | −79.1 | 76.4 | 7 | A |
| ASP | N | 178 | −25.6 | −78.4 | 74.5 | 8 | A |
| ASP | CA | 178 | −26.9 | −78.8 | 75.0 | 12 | A |
| ASP | CB | 178 | −27.9 | −78.5 | 73.8 | 11 | A |
| ASP | CG | 178 | −28.6 | −77.2 | 74.0 | 21 | A |
| ASP | OD1 | 178 | −29.2 | −76.9 | 75.1 | 24 | A |
| ASP | OD2 | 178 | −28.6 | −76.4 | 73.0 | 21 | A |
| ASP | C | 178 | −26.9 | −80.2 | 75.4 | 12 | A |
| ASP | O | 178 | −27.5 | −80.5 | 76.4 | 13 | A |
| SER | N | 179 | −26.3 | −81.1 | 74.6 | 11 | A |
| SER | CA | 179 | −26.2 | −82.5 | 75.0 | 9 | A |
| SER | CB | 179 | −25.6 | −83.3 | 73.9 | 9 | A |
| SER | OG | 179 | −26.4 | −83.3 | 72.7 | 1 | A |
| SER | C | 179 | −25.4 | −82.6 | 76.3 | 8 | A |
| SER | O | 179 | −25.8 | −83.2 | 77.2 | 8 | A |
| LEU | N | 180 | −24.2 | −82.0 | 76.3 | 8 | A |
| LEU | CA | 180 | −23.4 | −82.1 | 77.5 | 8 | A |
| LEU | CB | 180 | −22.1 | −81.2 | 77.4 | 7 | A |
| LEU | CG | 180 | −21.2 | −81.3 | 78.6 | 8 | A |
| LEU | CD1 | 180 | −20.5 | −82.6 | 78.6 | 6 | A |
| LEU | CD2 | 180 | −20.3 | −80.1 | 78.6 | 7 | A |
| LEU | C | 180 | −24.1 | −81.7 | 78.8 | 8 | A |
| LEU | O | 180 | −23.9 | −82.4 | 79.8 | 11 | A |
| VAL | N | 181 | −24.9 | −80.7 | 78.8 | 8 | A |
| VAL | CA | 181 | −25.5 | −80.3 | 80.0 | 9 | A |
| VAL | CB | 181 | −26.2 | −78.9 | 79.9 | 10 | A |
| VAL | CG1 | 181 | −27.1 | −78.6 | 81.1 | 8 | A |
| VAL | CG2 | 181 | −25.1 | −77.8 | 80.0 | 8 | A |
| VAL | C | 181 | −26.6 | −81.2 | 80.4 | 10 | A |
| VAL | O | 181 | −26.8 | −81.5 | 81.6 | 9 | A |
| LYS | N | 182 | −27.4 | −81.7 | 79.5 | 6 | A |
| LYS | CA | 182 | −28.5 | −82.6 | 79.8 | 5 | A |
| LYS | CB | 182 | −29.4 | −82.8 | 78.5 | 5 | A |
| LYS | CG | 182 | −29.9 | −81.5 | 78.0 | 4 | A |
| LYS | CD | 182 | −30.8 | −81.7 | 76.7 | 10 | A |
| LYS | CE | 182 | −32.2 | −82.1 | 77.1 | 10 | A |
| LYS | NZ | 182 | −33.0 | −81.0 | 77.7 | 7 | A |
| LYS | C | 182 | −27.9 | −84.0 | 80.2 | 4 | A |
| LYS | O | 182 | −28.6 | −84.7 | 81.0 | 5 | A |
| GLN | N | 183 | −26.8 | −84.4 | 79.8 | 2 | A |
| GLN | CA | 183 | −26.2 | −85.7 | 80.1 | 4 | A |
| GLN | CB | 183 | −25.5 | −86.2 | 78.9 | 3 | A |
| GLN | CG | 183 | −26.4 | −86.8 | 77.8 | 7 | A |
| GLN | CD | 183 | −25.7 | −87.0 | 76.5 | 13 | A |
| GLN | OE1 | 183 | −24.6 | −87.7 | 76.5 | 15 | A |
| GLN | NE2 | 183 | −26.3 | −86.5 | 75.4 | 13 | A |
| GLN | C | 183 | −25.3 | −85.7 | 81.3 | 8 | A |
| GLN | O | 183 | −24.9 | −86.8 | 81.8 | 13 | A |
| THR | N | 184 | −24.9 | −84.5 | 81.9 | 8 | A |
| THR | CA | 184 | −24.0 | −84.5 | 83.0 | 7 | A |
| THR | CB | 184 | −22.5 | −84.2 | 82.6 | 5 | A |
| THR | OG1 | 184 | −22.4 | −82.9 | 82.1 | 4 | A |
| THR | CG2 | 184 | −22.0 | −85.2 | 81.6 | 4 | A |
| THR | C | 184 | −24.4 | −83.4 | 84.0 | 8 | A |
| THR | O | 184 | −25.4 | −82.7 | 83.8 | 8 | A |
| HIS | N | 185 | −23.6 | −83.2 | 85.1 | 10 | A |
| HIS | CA | 185 | −23.9 | −82.2 | 86.0 | 12 | A |
| HIS | CB | 185 | −23.7 | −82.7 | 87.4 | 20 | A |
| HIS | CG | 185 | −24.7 | −83.7 | 87.9 | 28 | A |
| HIS | CD2 | 185 | −25.8 | −83.6 | 88.7 | 29 | A |
| HIS | ND1 | 185 | −24.6 | −85.0 | 87.5 | 31 | A |
| HIS | CE1 | 185 | −25.6 | −85.7 | 88.0 | 31 | A |
| HIS | NE2 | 185 | −26.3 | −84.9 | 88.7 | 32 | A |
| HIS | C | 185 | −23.1 | −80.9 | 85.8 | 10 | A |
| HIS | O | 185 | −23.2 | −80.0 | 86.6 | 10 | A |
| VAL | N | 186 | −22.5 | −80.8 | 84.6 | 6 | A |
| VAL | CA | 186 | −21.7 | −79.6 | 84.3 | 3 | A |
| VAL | CB | 186 | −21.1 | −79.8 | 82.9 | 3 | A |
| VAL | CG1 | 186 | −20.3 | −78.5 | 82.5 | 1 | A |
| VAL | CG2 | 186 | −20.1 | −80.9 | 82.9 | 1 | A |
| VAL | C | 186 | −22.7 | −78.5 | 84.2 | 3 | A |
| VAL | O | 186 | −23.7 | −78.5 | 83.5 | 5 | A |
| PRO | N | 187 | −22.4 | −77.4 | 85.0 | 3 | A |
| PRO | CD | 187 | −21.4 | −77.4 | 86.0 | 8 | A |
| PRO | CA | 187 | −23.2 | −76.2 | 85.0 | 4 | A |
| PRO | CB | 187 | −22.4 | −75.2 | 85.8 | 9 | A |
| PRO | CG | 187 | −21.8 | −76.1 | 86.8 | 8 | A |
| PRO | C | 187 | −23.5 | −75.8 | 83.6 | 1 | A |
| PRO | O | 187 | −22.8 | −76.0 | 82.7 | 1 | A |
| ASN | N | 188 | −24.7 | −75.1 | 83.4 | 1 | A |
| ASN | CA | 188 | −25.0 | −74.7 | 82.1 | 1 | A |
| ASN | CB | 188 | −26.5 | −74.5 | 81.9 | 6 | A |
| ASN | CG | 188 | −27.0 | −74.2 | 80.5 | 5 | A |
| ASN | OD1 | 188 | −26.2 | −74.3 | 79.5 | 7 | A |
| ASN | ND2 | 188 | −28.2 | −73.8 | 80.3 | 8 | A |
| ASN | C | 188 | −24.4 | −73.3 | 81.7 | 1 | A |
| ASN | O | 188 | −25.1 | −72.4 | 81.5 | 1 | A |
| LEU | N | 189 | −23.1 | −73.3 | 81.5 | 1 | A |
| LEU | CA | 189 | −22.4 | −72.1 | 81.1 | 1 | A |
| LEU | CB | 189 | −22.6 | −71.0 | 82.1 | 3 | A |
| LEU | CG | 189 | −21.9 | −71.2 | 83.4 | 6 | A |
| LEU | CD1 | 189 | −20.7 | −70.2 | 83.4 | 4 | A |
| LEU | CD2 | 189 | −22.8 | −70.8 | 84.6 | 7 | A |
| LEU | C | 189 | −20.9 | −72.3 | 80.9 | 1 | A |
| LEU | O | 189 | −20.3 | −73.1 | 81.6 | 1 | A |
| PHE | N | 190 | −20.3 | −71.5 | 80.0 | 1 | A |
| PHE | CA | 190 | −18.9 | −71.6 | 79.8 | 1 | A |
| PHE | CB | 190 | −18.6 | −72.4 | 78.5 | 3 | A |
| PHE | CG | 190 | −19.1 | −71.7 | 77.2 | 2 | A |
| PHE | CD1 | 190 | −20.4 | −71.9 | 76.8 | 5 | A |
| PHE | CD2 | 190 | −18.2 | −71.0 | 76.5 | 2 | A |
| PHE | CE1 | 190 | −20.8 | −71.3 | 75.6 | 1 | A |
| PHE | CE2 | 190 | −18.6 | −70.3 | 75.3 | 3 | A |
| PHE | CZ | 190 | −19.9 | −70.5 | 74.9 | 3 | A |
| PHE | C | 190 | −18.3 | −70.2 | 79.6 | 1 | A |
| PHE | O | 190 | −19.0 | −69.3 | 79.2 | 1 | A |
| SER | N | 191 | −17.0 | −70.0 | 79.8 | 3 | A |
| SER | CA | 191 | −16.4 | −68.7 | 79.7 | 4 | A |
| SER | CB | 191 | −16.0 | −68.2 | 81.1 | 6 | A |
| SER | OG | 191 | −15.0 | −69.0 | 81.7 | 13 | A |
| SER | C | 191 | −15.1 | −68.9 | 78.8 | 3 | A |
| SER | O | 191 | −14.4 | −69.9 | 78.9 | 2 | A |
| LEU | N | 192 | −14.8 | −67.9 | 78.0 | 1 | A |
| LEU | CA | 192 | −13.6 | −68.0 | 77.1 | 1 | A |
| LEU | CB | 192 | −14.1 | −67.9 | 75.7 | 1 | A |
| LEU | CG | 192 | −14.7 | −69.1 | 75.0 | 1 | A |
| LEU | CD1 | 192 | −15.3 | −68.7 | 73.7 | 1 | A |
| LEU | CD2 | 192 | −13.6 | −70.1 | 74.7 | 2 | A |
| LEU | C | 192 | −12.7 | −66.8 | 77.4 | 1 | A |
| LEU | O | 192 | −13.2 | −65.7 | 77.6 | 1 | A |
| GLN | N | 193 | −11.4 | −67.0 | 77.4 | 1 | A |
| GLN | CA | 193 | −10.5 | −66.0 | 77.6 | 1 | A |
| GLN | CB | 193 | −9.6 | −66.2 | 78.8 | 1 | A |
| GLN | CG | 193 | −8.6 | −65.0 | 79.0 | 1 | A |
| GLN | CD | 193 | −7.4 | −65.4 | 79.8 | 1 | A |
| GLN | OE1 | 193 | −6.7 | −66.4 | 79.4 | 2 | A |
| GLN | NE2 | 193 | −7.1 | −64.7 | 80.9 | 1 | A |
| GLN | C | 193 | −9.6 | −66.1 | 76.4 | 1 | A |
| GLN | O | 193 | −8.6 | −66.8 | 76.5 | 1 | A |
| LEU | N | 194 | −9.9 | −65.4 | 75.3 | 3 | A |
| LEU | CA | 194 | −9.0 | −65.5 | 74.1 | 3 | A |
| LEU | CB | 194 | −9.9 | −65.3 | 72.9 | 2 | A |
| LEU | CG | 194 | −11.0 | −66.2 | 72.7 | 1 | A |
| LEU | CD1 | 194 | −11.9 | −65.9 | 71.6 | 1 | A |
| LEU | CD2 | 194 | −10.5 | −67.6 | 72.6 | 1 | A |
| LEU | C | 194 | −7.9 | −64.5 | 74.3 | 5 | A |
| LEU | O | 194 | −8.2 | −63.3 | 74.5 | 9 | A |
| CYS | N | 195 | −6.7 | −64.9 | 74.1 | 5 | A |
| CYS | CA | 195 | −5.6 | −64.0 | 74.2 | 9 | A |
| CYS | C | 195 | −4.9 | −63.9 | 72.9 | 12 | A |
| CYS | O | 195 | −4.5 | −64.8 | 72.3 | 14 | A |
| CYS | CB | 195 | −4.5 | −64.5 | 75.2 | 10 | A |
| CYS | SG | 195 | −5.2 | −65.0 | 76.8 | 16 | A |
| GLY | N | 196 | −4.8 | −62.7 | 72.3 | 16 | A |
| GLY | CA | 196 | −4.2 | −62.5 | 71.1 | 17 | A |
| GLY | C | 196 | −2.7 | −62.6 | 71.2 | 18 | A |
| GLY | O | 196 | −2.2 | −62.9 | 72.4 | 17 | A |
| ALA | N | 197 | −1.9 | −62.4 | 70.2 | 19 | A |
| ALA | CA | 197 | −0.5 | −62.5 | 70.3 | 21 | A |
| ALA | CB | 197 | 0.1 | −62.9 | 68.9 | 20 | A |
| ALA | C | 197 | 0.2 | −61.2 | 70.8 | 23 | A |
| ALA | O | 197 | 0.6 | −61.1 | 71.9 | 24 | A |
| GLY | N | 198 | 0.2 | −60.2 | 69.9 | 27 | A |
| GLY | CA | 198 | 0.8 | −58.9 | 70.3 | 31 | A |
| GLY | C | 198 | 2.2 | −58.9 | 69.6 | 32 | A |
| GLY | O | 198 | 2.8 | −57.9 | 69.2 | 38 | A |
| PHE | N | 199 | 2.7 | −60.2 | 69.5 | 33 | A |
| PHE | CA | 199 | 4.0 | −60.4 | 68.8 | 37 | A |
| PHE | CB | 199 | 5.0 | −61.0 | 69.8 | 35 | A |
| PHE | CG | 199 | 4.6 | −60.9 | 71.3 | 36 | A |
| PHE | CD1 | 199 | 4.4 | −59.7 | 71.9 | 33 | A |
| PHE | CD2 | 199 | 4.4 | −62.1 | 72.0 | 33 | A |
| PHE | CE1 | 199 | 4.0 | −59.7 | 73.3 | 30 | A |
| PHE | CE2 | 199 | 4.1 | −62.1 | 73.3 | 32 | A |
| PHE | CZ | 199 | 3.9 | −60.9 | 74.0 | 30 | A |
| PHE | C | 199 | 3.7 | −61.4 | 67.7 | 38 | A |
| PHE | O | 199 | 2.8 | −62.2 | 67.8 | 38 | A |
| PRO | N | 200 | 4.6 | −61.5 | 66.7 | 40 | A |
| PRO | CD | 200 | 5.7 | −60.6 | 66.3 | 41 | A |
| PRO | CA | 200 | 4.3 | −62.4 | 65.6 | 39 | A |
| PRO | CB | 200 | 5.3 | −62.0 | 64.5 | 42 | A |
| PRO | CG | 200 | 6.5 | −61.4 | 65.3 | 41 | A |
| PRO | C | 200 | 4.4 | −63.9 | 65.9 | 38 | A |
| PRO | O | 200 | 5.2 | −64.3 | 66.8 | 36 | A |
| LEU | N | 201 | 3.6 | −64.7 | 65.3 | 37 | A |
| LEU | CA | 201 | 3.6 | −66.2 | 65.4 | 34 | A |
| LEU | CB | 201 | 2.6 | −66.6 | 66.5 | 30 | A |
| LEU | CG | 201 | 2.8 | −66.3 | 67.9 | 33 | A |
| LEU | CD1 | 201 | 1.9 | −67.2 | 68.8 | 29 | A |
| LEU | CD2 | 201 | 4.3 | −66.6 | 68.3 | 28 | A |
| LEU | C | 201 | 3.1 | −66.8 | 64.1 | 34 | A |
| LEU | O | 201 | 1.9 | −66.9 | 63.8 | 31 | A |
| ASN | N | 202 | 4.1 | −67.2 | 63.3 | 34 | A |
| ASN | CA | 202 | 3.8 | −67.9 | 62.0 | 36 | A |
| ASN | CB | 202 | 5.1 | −68.2 | 61.2 | 35 | A |
| ASN | CG | 202 | 5.9 | −69.4 | 61.9 | 37 | A |
| ASN | OD1 | 202 | 6.4 | −69.3 | 63.0 | 36 | A |
| ASN | ND2 | 202 | 6.1 | −70.5 | 61.1 | 32 | A |
| ASN | C | 202 | 3.0 | −69.2 | 62.2 | 37 | A |
| ASN | O | 202 | 2.2 | −69.3 | 63.2 | 39 | A |
| GLN | N | 203 | 3.1 | −70.1 | 61.3 | 38 | A |
| GLN | CA | 203 | 2.4 | −71.4 | 61.5 | 38 | A |
| GLN | CB | 203 | 2.4 | −72.2 | 60.2 | 38 | A |
| GLN | CG | 203 | 1.5 | −73.5 | 60.2 | 39 | A |
| GLN | CD | 203 | 2.2 | −74.7 | 60.7 | 40 | A |
| GLN | OE1 | 203 | 2.2 | −75.0 | 61.9 | 39 | A |
| GLN | NE2 | 203 | 2.8 | −75.4 | 59.7 | 40 | A |
| GLN | C | 203 | 3.0 | −72.3 | 62.6 | 39 | A |
| GLN | O | 203 | 2.2 | −72.8 | 63.4 | 38 | A |
| SER | N | 204 | 4.3 | −72.4 | 62.6 | 39 | A |
| SER | CA | 204 | 5.0 | −73.2 | 63.7 | 41 | A |
| SER | CB | 204 | 6.4 | −73.5 | 63.1 | 41 | A |
| SER | OG | 204 | 7.0 | −74.5 | 64.0 | 40 | A |
| SER | C | 204 | 5.1 | −72.5 | 65.1 | 43 | A |
| SER | O | 204 | 6.0 | −72.8 | 65.9 | 44 | A |
| GLU | N | 205 | 4.1 | −71.7 | 65.4 | 43 | A |
| GLU | CA | 205 | 4.0 | −71.0 | 66.7 | 41 | A |
| GLU | CB | 205 | 4.3 | −69.5 | 66.5 | 37 | A |
| GLU | CG | 205 | 5.7 | −69.1 | 66.2 | 36 | A |
| GLU | CD | 205 | 6.7 | −69.4 | 67.3 | 40 | A |
| GLU | OE1 | 205 | 6.9 | −70.5 | 67.8 | 37 | A |
| GLU | OE2 | 205 | 7.3 | −68.4 | 67.8 | 37 | A |
| GLU | C | 205 | 2.7 | −71.2 | 67.4 | 41 | A |
| GLU | O | 205 | 2.2 | −70.4 | 68.2 | 39 | A |
| VAL | N | 206 | 2.1 | −72.4 | 67.1 | 40 | A |
| VAL | CA | 206 | 0.8 | −72.8 | 67.7 | 38 | A |
| VAL | CB | 206 | −0.3 | −72.9 | 66.6 | 34 | A |
| VAL | CG1 | 206 | −1.7 | −72.6 | 67.2 | 30 | A |
| VAL | CG2 | 206 | 0.0 | −72.1 | 65.4 | 33 | A |
| VAL | C | 206 | 0.8 | −74.1 | 68.5 | 38 | A |
| VAL | O | 206 | 0.5 | −74.2 | 69.7 | 35 | A |
| LEU | N | 207 | 1.3 | −75.1 | 67.8 | 39 | A |
| LEU | CA | 207 | 1.4 | −76.5 | 68.3 | 39 | A |
| LEU | CB | 207 | 2.0 | −77.4 | 67.3 | 36 | A |
| LEU | CG | 207 | 1.3 | −77.6 | 65.9 | 34 | A |
| LEU | CD1 | 207 | 1.6 | −76.5 | 64.9 | 27 | A |
| LEU | CD2 | 207 | 1.8 | −79.0 | 65.3 | 32 | A |
| LEU | C | 207 | 2.3 | −76.5 | 69.6 | 41 | A |
| LEU | O | 207 | 2.1 | −77.3 | 70.5 | 40 | A |
| ALA | N | 208 | 3.4 | −75.7 | 69.5 | 43 | A |
| ALA | CA | 208 | 4.3 | −75.6 | 70.6 | 43 | A |
| ALA | CB | 208 | 5.7 | −75.3 | 70.1 | 41 | A |
| ALA | C | 208 | 3.9 | −74.7 | 71.7 | 44 | A |
| ALA | O | 208 | 4.6 | −74.5 | 72.8 | 44 | A |
| SER | N | 209 | 2.8 | −73.9 | 71.5 | 44 | A |
| SER | CA | 209 | 2.3 | −73.0 | 72.5 | 42 | A |
| SER | CB | 209 | 2.7 | −71.5 | 72.0 | 41 | A |
| SER | OG | 209 | 4.0 | −71.2 | 72.2 | 35 | A |
| SER | C | 209 | 0.8 | −73.0 | 72.9 | 41 | A |
| SER | O | 209 | 0.2 | −74.1 | 72.9 | 43 | A |
| VAL | N | 210 | 0.3 | −71.8 | 73.3 | 41 | A |
| VAL | CA | 210 | −1.1 | −71.7 | 73.7 | 35 | A |
| VAL | CB | 210 | −1.2 | −72.0 | 75.3 | 32 | A |
| VAL | CG1 | 210 | −2.6 | −71.8 | 75.7 | 29 | A |
| VAL | CG2 | 210 | −0.8 | −73.4 | 75.5 | 26 | A |
| VAL | C | 210 | −1.6 | −70.2 | 73.4 | 34 | A |
| VAL | O | 210 | −0.9 | −69.2 | 73.7 | 35 | A |
| GLY | N | 211 | −2.8 | −70.1 | 72.9 | 28 | A |
| GLY | CA | 211 | −3.5 | −68.9 | 72.6 | 17 | A |
| GLY | C | 211 | −4.4 | −68.4 | 73.7 | 13 | A |
| GLY | O | 211 | −4.8 | −67.2 | 73.8 | 12 | A |
| GLY | N | 212 | −4.9 | −69.3 | 74.5 | 11 | A |
| GLY | CA | 212 | −5.9 | −68.9 | 75.6 | 9 | A |
| GLY | C | 212 | −6.6 | −70.0 | 76.2 | 7 | A |
| GLY | O | 212 | −6.2 | −71.2 | 76.1 | 8 | A |
| SER | N | 213 | −7.7 | −69.7 | 77.0 | 4 | A |
| SER | CA | 213 | −8.4 | −70.8 | 77.7 | 2 | A |
| SER | CB | 213 | −8.0 | −70.8 | 79.1 | 1 | A |
| SER | OG | 213 | −7.6 | −69.6 | 79.7 | 1 | A |
| SER | C | 213 | −10.0 | −70.7 | 77.6 | 1 | A |
| SER | O | 213 | −10.6 | −69.7 | 77.6 | 3 | A |
| MET | N | 214 | −10.6 | −71.9 | 77.6 | 1 | A |
| MET | CA | 214 | −12.0 | −72.0 | 77.6 | 1 | A |
| MET | CB | 214 | −12.5 | −72.8 | 76.3 | 1 | A |
| MET | CG | 214 | −14.0 | −73.3 | 76.5 | 1 | A |
| MET | SD | 214 | −14.7 | −74.0 | 75.0 | 1 | A |
| MET | CE | 214 | −14.3 | −75.7 | 75.3 | 1 | A |
| MET | C | 214 | −12.4 | −72.9 | 78.8 | 2 | A |
| MET | O | 214 | −12.3 | −74.1 | 78.8 | 2 | A |
| ILE | N | 215 | −13.0 | −72.2 | 79.8 | 2 | A |
| ILE | CA | 215 | −13.5 | −72.8 | 81.0 | 1 | A |
| ILE | CB | 215 | −13.5 | −71.8 | 82.2 | 1 | A |
| ILE | CG2 | 215 | −14.0 | −72.5 | 83.4 | 3 | A |
| ILE | CG1 | 215 | −12.1 | −71.3 | 82.3 | 1 | A |
| ILE | CD1 | 215 | −11.0 | −72.3 | 82.3 | 1 | A |
| ILE | C | 215 | −14.9 | −73.4 | 80.9 | 1 | A |
| ILE | O | 215 | −15.8 | −72.6 | 80.7 | 1 | A |
| ILE | N | 216 | −15.0 | −74.7 | 80.9 | 1 | A |
| ILE | CA | 216 | −16.3 | −75.4 | 80.8 | 4 | A |
| ILE | CB | 216 | −16.2 | −76.8 | 80.2 | 6 | A |
| ILE | CG2 | 216 | −17.6 | −77.4 | 80.1 | 4 | A |
| ILE | CG1 | 216 | −15.5 | −76.7 | 78.8 | 9 | A |
| ILE | CD1 | 216 | −16.2 | −75.9 | 77.8 | 16 | A |
| ILE | C | 216 | −17.0 | −75.5 | 82.1 | 3 | A |
| ILE | O | 216 | −16.5 | −76.1 | 83.1 | 1 | A |
| GLY | N | 217 | −18.2 | −74.9 | 82.3 | 5 | A |
| GLY | CA | 217 | −19.0 | −75.1 | 83.5 | 3 | A |
| GLY | C | 217 | −18.7 | −74.1 | 84.6 | 1 | A |
| GLY | O | 217 | −18.9 | −74.5 | 85.8 | 3 | A |
| GLY | N | 218 | −18.4 | −72.9 | 84.3 | 1 | A |
| GLY | CA | 218 | −18.1 | −71.9 | 85.4 | 3 | A |
| GLY | C | 218 | −17.1 | −70.9 | 85.1 | 3 | A |
| GLY | O | 218 | −16.5 | −70.8 | 84.0 | 2 | A |
| ILE | N | 219 | −16.9 | −70.0 | 86.1 | 5 | A |
| ILE | CA | 219 | −15.9 | −68.9 | 86.0 | 6 | A |
| ILE | CB | 219 | −16.6 | −67.6 | 86.4 | 2 | A |
| ILE | CG2 | 219 | −15.8 | −66.4 | 85.9 | 5 | A |
| ILE | CG1 | 219 | −18.0 | −67.6 | 85.9 | 1 | A |
| ILE | CD1 | 219 | −18.8 | −66.5 | 86.5 | 1 | A |
| ILE | C | 219 | −14.6 | −69.1 | 86.7 | 6 | A |
| ILE | O | 219 | −14.6 | −69.7 | 87.8 | 12 | A |
| ASP | N | 220 | −13.5 | −68.6 | 86.2 | 7 | A |
| ASP | CA | 220 | −12.2 | −68.7 | 86.8 | 8 | A |
| ASP | CB | 220 | −11.2 | −69.4 | 86.0 | 3 | A |
| ASP | CG | 220 | −9.9 | −69.6 | 86.6 | 1 | A |
| ASP | OD1 | 220 | −9.5 | −70.8 | 86.9 | 1 | A |
| ASP | OD2 | 220 | −9.2 | −68.6 | 86.9 | 1 | A |
| ASP | C | 220 | −11.7 | −67.3 | 87.2 | 11 | A |
| ASP | O | 220 | −11.1 | −66.6 | 86.3 | 8 | A |
| HIS | N | 221 | −11.9 | −66.9 | 88.4 | 16 | A |
| HIS | CA | 221 | −11.5 | −65.6 | 88.9 | 18 | A |
| HIS | CB | 221 | −11.9 | −65.5 | 90.4 | 26 | A |
| HIS | CG | 221 | −13.3 | −65.4 | 90.6 | 37 | A |
| HIS | CD2 | 221 | −14.3 | −66.3 | 90.6 | 35 | A |
| HIS | ND1 | 221 | −14.0 | −64.2 | 91.0 | 39 | A |
| HIS | CE1 | 221 | −15.3 | −64.5 | 91.2 | 38 | A |
| HIS | NE2 | 221 | −15.5 | −65.7 | 90.9 | 37 | A |
| HIS | C | 221 | −10.1 | −65.2 | 88.6 | 14 | A |
| HIS | O | 221 | −9.7 | −64.0 | 88.7 | 13 | A |
| SER | N | 222 | −9.3 | −66.2 | 88.2 | 10 | A |
| SER | CA | 222 | −7.9 | −65.9 | 87.9 | 8 | A |
| SER | CB | 222 | −7.0 | −67.1 | 88.3 | 9 | A |
| SER | OG | 222 | −6.9 | −68.0 | 87.3 | 8 | A |
| SER | C | 222 | −7.6 | −65.5 | 86.5 | 6 | A |
| SER | O | 222 | −6.4 | −65.6 | 86.0 | 3 | A |
| LEU | N | 223 | −8.6 | −65.2 | 85.8 | 7 | A |
| LEU | CA | 223 | −8.5 | −64.9 | 84.4 | 4 | A |
| LEU | CB | 223 | −9.3 | −65.8 | 83.5 | 1 | A |
| LEU | CG | 223 | −8.9 | −67.3 | 83.6 | 1 | A |
| LEU | CD1 | 223 | −9.9 | −68.2 | 83.0 | 1 | A |
| LEU | CD2 | 223 | −7.6 | −67.5 | 82.8 | 1 | A |
| LEU | C | 223 | −8.9 | −63.4 | 84.1 | 2 | A |
| LEU | O | 223 | −8.7 | −62.9 | 83.0 | 1 | A |
| TYR | N | 224 | −9.5 | −62.8 | 85.1 | 1 | A |
| TYR | CA | 224 | −9.9 | −61.4 | 84.9 | 1 | A |
| TYR | CB | 224 | −11.4 | −61.3 | 84.6 | 1 | A |
| TYR | CG | 224 | −12.4 | −61.7 | 85.6 | 1 | A |
| TYR | CD1 | 224 | −12.6 | −63.0 | 86.0 | 1 | A |
| TYR | CE1 | 224 | −13.6 | −63.3 | 87.0 | 1 | A |
| TYR | CD2 | 224 | −13.2 | −60.7 | 86.2 | 1 | A |
| TYR | CE2 | 224 | −14.2 | −61.0 | 87.1 | 1 | A |
| TYR | CZ | 224 | −14.3 | −62.3 | 87.5 | 1 | A |
| TYR | OH | 224 | −15.3 | −62.7 | 88.4 | 3 | A |
| TYR | C | 224 | −9.7 | −60.5 | 86.1 | 1 | A |
| TYR | O | 224 | −9.7 | −60.9 | 87.3 | 1 | A |
| THR | N | 225 | −9.5 | −59.2 | 85.8 | 1 | A |
| THR | CA | 225 | −9.2 | −58.2 | 86.9 | 3 | A |
| THR | CB | 225 | −7.9 | −57.4 | 86.6 | 2 | A |
| THR | OG1 | 225 | −8.0 | −56.8 | 85.3 | 2 | A |
| THR | CG2 | 225 | −6.7 | −58.3 | 86.7 | 4 | A |
| THR | C | 225 | −10.4 | −57.2 | 86.8 | 5 | A |
| THR | O | 225 | −10.9 | −56.9 | 85.7 | 8 | A |
| GLY | N | 226 | −10.8 | −56.7 | 87.9 | 5 | A |
| GLY | CA | 226 | −12.0 | −55.8 | 87.9 | 2 | A |
| GLY | C | 226 | −13.3 | −56.6 | 88.1 | 1 | A |
| GLY | O | 226 | −13.2 | −57.7 | 88.7 | 1 | A |
| SER | N | 227 | −14.4 | −56.0 | 87.8 | 1 | A |
| SER | CA | 227 | −15.7 | −56.6 | 88.0 | 1 | A |
| SER | CB | 227 | −16.7 | −55.6 | 88.6 | 1 | A |
| SER | OG | 227 | −16.2 | −55.0 | 89.8 | 4 | A |
| SER | C | 227 | −16.2 | −57.2 | 86.7 | 1 | A |
| SER | O | 227 | −15.7 | −57.0 | 85.6 | 1 | A |
| LEU | N | 228 | −17.4 | −57.9 | 86.8 | 2 | A |
| LEU | CA | 228 | −18.0 | −58.5 | 85.7 | 1 | A |
| LEU | CB | 228 | −18.3 | −60.0 | 85.9 | 1 | A |
| LEU | CG | 228 | −17.2 | −61.1 | 85.7 | 1 | A |
| LEU | CD1 | 228 | −17.7 | −62.5 | 86.1 | 1 | A |
| LEU | CD2 | 228 | −16.9 | −61.0 | 84.2 | 1 | A |
| LEU | C | 228 | −19.4 | −57.9 | 85.4 | 1 | A |
| LEU | O | 228 | −20.2 | −57.9 | 86.3 | 1 | A |
| TRP | N | 229 | −19.5 | −57.2 | 84.3 | 1 | A |
| TRP | CA | 229 | −20.8 | −56.6 | 83.9 | 1 | A |
| TRP | CB | 229 | −20.5 | −55.2 | 83.4 | 2 | A |
| TRP | CG | 229 | −20.0 | −54.2 | 84.4 | 1 | A |
| TRP | CD2 | 229 | −20.8 | −53.2 | 85.0 | 2 | A |
| TRP | CE2 | 229 | −19.9 | −52.5 | 85.9 | 1 | A |
| TRP | CE3 | 229 | −22.1 | −52.8 | 85.0 | 2 | A |
| TRP | CD1 | 229 | −18.8 | −54.1 | 84.9 | 1 | A |
| TRP | NE1 | 229 | −18.7 | −53.1 | 85.8 | 1 | A |
| TRP | CZ2 | 229 | −20.3 | −51.4 | 86.7 | 3 | A |
| TRP | CZ3 | 229 | −22.5 | −51.7 | 85.7 | 1 | A |
| TRP | CH2 | 229 | −21.6 | −51.0 | 86.6 | 2 | A |
| TRP | C | 229 | −21.5 | −57.4 | 82.9 | 1 | A |
| TRP | O | 229 | −20.9 | −57.7 | 81.9 | 1 | A |
| TYR | N | 230 | −22.8 | −57.7 | 83.2 | 1 | A |
| TYR | CA | 230 | −23.5 | −58.5 | 82.3 | 2 | A |
| TYR | CB | 230 | −24.3 | −59.6 | 83.1 | 1 | A |
| TYR | CG | 230 | −23.5 | −60.6 | 83.8 | 1 | A |
| TYR | CD1 | 230 | −23.0 | −60.3 | 85.1 | 1 | A |
| TYR | CE1 | 230 | −22.2 | −61.2 | 85.8 | 1 | A |
| TYR | CD2 | 230 | −23.1 | −61.8 | 83.2 | 1 | A |
| TYR | CE2 | 230 | −22.3 | −62.7 | 83.9 | 1 | A |
| TYR | CZ | 230 | −21.8 | −62.3 | 85.2 | 1 | A |
| TYR | OH | 230 | −21.0 | −63.2 | 85.8 | 1 | A |
| TYR | C | 230 | −24.5 | −57.8 | 81.4 | 3 | A |
| TYR | O | 230 | −24.9 | −56.7 | 81.7 | 4 | A |
| THR | N | 231 | −24.7 | −58.4 | 80.2 | 3 | A |
| THR | CA | 231 | −25.6 | −57.8 | 79.2 | 1 | A |
| THR | CB | 231 | −24.9 | −57.3 | 78.0 | 1 | A |
| THR | OG1 | 231 | −25.8 | −56.9 | 77.0 | 1 | A |
| THR | CG2 | 231 | −24.0 | −58.5 | 77.4 | 1 | A |
| THR | C | 231 | −26.6 | −58.9 | 78.8 | 3 | A |
| THR | O | 231 | −26.2 | −60.1 | 78.6 | 3 | A |
| PRO | N | 232 | −27.9 | −58.6 | 78.7 | 3 | A |
| PRO | CD | 232 | −28.4 | −57.3 | 78.9 | 3 | A |
| PRO | CA | 232 | −28.9 | −59.6 | 78.3 | 4 | A |
| PRO | CB | 232 | −30.2 | −58.7 | 78.3 | 3 | A |
| PRO | CG | 232 | −29.8 | −57.6 | 79.2 | 3 | A |
| PRO | C | 232 | −28.7 | −60.3 | 77.0 | 7 | A |
| PRO | O | 232 | −28.3 | −59.6 | 76.1 | 13 | A |
| ILE | N | 233 | −29.0 | −61.6 | 76.9 | 6 | A |
| ILE | CA | 233 | −28.9 | −62.3 | 75.7 | 3 | A |
| ILE | CB | 233 | −28.7 | −63.8 | 75.8 | 1 | A |
| ILE | CG2 | 233 | −28.7 | −64.5 | 74.5 | 1 | A |
| ILE | CG1 | 233 | −27.3 | −64.1 | 76.5 | 3 | A |
| ILE | CD1 | 233 | −26.9 | −65.6 | 76.4 | 1 | A |
| ILE | C | 233 | −30.2 | −62.1 | 75.1 | 2 | A |
| ILE | O | 233 | −31.2 | −62.6 | 75.6 | 3 | A |
| ARG | N | 234 | −30.3 | −61.3 | 74.0 | 2 | A |
| ARG | CA | 234 | −31.6 | −61.0 | 73.4 | 1 | A |
| ARG | CB | 234 | −31.3 | −60.3 | 72.1 | 1 | A |
| ARG | CG | 234 | −32.0 | −58.9 | 72.1 | 1 | A |
| ARG | CD | 234 | −32.4 | −58.5 | 70.7 | 4 | A |
| ARG | NE | 234 | −33.6 | −57.8 | 70.7 | 6 | A |
| ARG | CZ | 234 | −34.3 | −57.5 | 69.6 | 5 | A |
| ARG | NH1 | 234 | −33.9 | −57.9 | 68.4 | 2 | A |
| ARG | NH2 | 234 | −35.4 | −56.8 | 69.8 | 9 | A |
| ARG | C | 234 | −32.4 | −62.2 | 73.1 | 1 | A |
| ARG | O | 234 | −33.4 | −62.5 | 73.8 | 3 | A |
| ARG | N | 235 | −32.1 | −63.0 | 72.0 | 1 | A |
| ARG | CA | 235 | −32.9 | −64.2 | 71.7 | 1 | A |
| ARG | CB | 235 | −33.5 | −64.0 | 70.3 | 4 | A |
| ARG | CG | 235 | −34.7 | −64.9 | 70.0 | 10 | A |
| ARG | CD | 235 | −34.7 | −65.5 | 68.7 | 10 | A |
| ARG | NE | 235 | −34.5 | −64.5 | 67.6 | 11 | A |
| ARG | CZ | 235 | −34.4 | −64.9 | 66.3 | 10 | A |
| ARG | NH1 | 235 | −34.5 | −66.2 | 65.9 | 9 | A |
| ARG | NH2 | 235 | −34.3 | −63.9 | 65.4 | 10 | A |
| ARG | C | 235 | −31.9 | −65.3 | 71.6 | 1 | A |
| ARG | O | 235 | −30.8 | −65.2 | 71.1 | 3 | A |
| GLU | N | 236 | −32.3 | −66.5 | 72.2 | 2 | A |
| GLU | CA | 236 | −31.4 | −67.6 | 72.2 | 3 | A |
| GLU | CB | 236 | −31.8 | −68.6 | 73.3 | 7 | A |
| GLU | CG | 236 | −31.7 | −68.0 | 74.7 | 13 | A |
| GLU | CD | 236 | −31.9 | −69.1 | 75.8 | 15 | A |
| GLU | OE1 | 236 | −33.0 | −69.7 | 75.8 | 15 | A |
| GLU | OE2 | 236 | −30.9 | −69.3 | 76.6 | 16 | A |
| GLU | C | 236 | −31.2 | −68.3 | 70.9 | 1 | A |
| GLU | O | 236 | −31.6 | −69.4 | 70.8 | 3 | A |
| TRP | N | 237 | −30.5 | −67.7 | 69.9 | 1 | A |
| TRP | CA | 237 | −30.2 | −68.3 | 68.7 | 1 | A |
| TRP | CB | 237 | −31.0 | −67.7 | 67.5 | 1 | A |
| TRP | CG | 237 | −30.9 | −66.2 | 67.3 | 3 | A |
| TRP | CD2 | 237 | −31.1 | −65.5 | 66.1 | 1 | A |
| TRP | CE2 | 237 | −31.1 | −64.2 | 66.4 | 1 | A |
| TRP | CE3 | 237 | −31.2 | −65.9 | 64.8 | 1 | A |
| TRP | CD1 | 237 | −30.8 | −65.3 | 68.3 | 5 | A |
| TRP | NE1 | 237 | −30.9 | −64.0 | 67.7 | 1 | A |
| TRP | CZ2 | 237 | −31.2 | −63.2 | 65.4 | 1 | A |
| TRP | CZ3 | 237 | −31.3 | −65.0 | 63.8 | 2 | A |
| TRP | CH2 | 237 | −31.3 | −63.6 | 64.1 | 3 | A |
| TRP | C | 237 | −28.7 | −68.1 | 68.5 | 1 | A |
| TRP | O | 237 | −27.9 | −69.0 | 68.8 | 2 | A |
| TYR | N | 238 | −28.3 | −66.9 | 68.1 | 2 | A |
| TYR | CA | 238 | −26.9 | −66.6 | 68.0 | 2 | A |
| TYR | CB | 238 | −26.6 | −65.5 | 67.0 | 2 | A |
| TYR | CG | 238 | −26.5 | −65.9 | 65.5 | 1 | A |
| TYR | CD1 | 238 | −25.4 | −66.6 | 65.0 | 3 | A |
| TYR | CE1 | 238 | −25.3 | −66.9 | 63.7 | 5 | A |
| TYR | CD2 | 238 | −27.5 | −65.6 | 64.7 | 3 | A |
| TYR | CE2 | 238 | −27.5 | −65.9 | 63.3 | 8 | A |
| TYR | CZ | 238 | −26.3 | −66.5 | 62.8 | 6 | A |
| TYR | OH | 238 | −26.2 | −66.8 | 61.5 | 10 | A |
| TYR | C | 238 | −26.8 | −66.0 | 69.4 | 3 | A |
| TYR | O | 238 | −27.8 | −65.7 | 70.0 | 6 | A |
| TYR | N | 239 | −25.6 | −65.7 | 69.9 | 3 | A |
| TYR | CA | 239 | −25.5 | −65.1 | 71.2 | 3 | A |
| TYR | CB | 239 | −24.2 | −65.3 | 71.9 | 2 | A |
| TYR | CG | 239 | −24.0 | −66.7 | 72.4 | 1 | A |
| TYR | CD1 | 239 | −23.3 | −67.7 | 71.7 | 1 | A |
| TYR | CE1 | 239 | −23.2 | −69.0 | 72.1 | 1 | A |
| TYR | CD2 | 239 | −24.5 | −67.1 | 73.6 | 1 | A |
| TYR | CE2 | 239 | −24.4 | −68.4 | 74.1 | 1 | A |
| TYR | CZ | 239 | −23.7 | −69.3 | 73.4 | 1 | A |
| TYR | OH | 239 | −23.6 | −70.6 | 73.8 | 2 | A |
| TYR | C | 239 | −25.7 | −63.6 | 71.0 | 3 | A |
| TYR | O | 239 | −24.8 | −62.8 | 71.1 | 1 | A |
| GLU | N | 240 | −26.9 | −63.2 | 70.6 | 3 | A |
| GLU | CA | 240 | −27.2 | −61.8 | 70.3 | 4 | A |
| GLU | CB | 240 | −28.6 | −61.7 | 69.6 | 2 | A |
| GLU | CG | 240 | −29.1 | −60.3 | 69.4 | 2 | A |
| GLU | CD | 240 | −30.3 | −60.1 | 68.5 | 1 | A |
| GLU | OE1 | 240 | −31.2 | −61.0 | 68.6 | 1 | A |
| GLU | OE2 | 240 | −30.3 | −59.2 | 67.6 | 4 | A |
| GLU | C | 240 | −27.2 | −60.9 | 71.5 | 3 | A |
| GLU | O | 240 | −27.6 | −61.3 | 72.6 | 1 | A |
| VAL | N | 241 | −26.7 | −59.7 | 71.3 | 1 | A |
| VAL | CA | 241 | −26.6 | −58.7 | 72.3 | 1 | A |
| VAL | CB | 241 | −25.2 | −58.5 | 72.8 | 1 | A |
| VAL | CG1 | 241 | −24.7 | −59.7 | 73.5 | 1 | A |
| VAL | CG2 | 241 | −24.3 | −58.1 | 71.6 | 1 | A |
| VAL | C | 241 | −27.1 | −57.4 | 71.6 | 1 | A |
| VAL | O | 241 | −27.3 | −57.4 | 70.4 | 1 | A |
| ILE | N | 242 | −27.3 | −56.3 | 72.4 | 1 | A |
| ILE | CA | 242 | −27.8 | −55.1 | 71.9 | 1 | A |
| ILE | CB | 242 | −29.1 | −54.7 | 72.4 | 1 | A |
| ILE | CG2 | 242 | −29.4 | −53.2 | 72.2 | 1 | A |
| ILE | CG1 | 242 | −30.2 | −55.6 | 71.8 | 1 | A |
| ILE | CD1 | 242 | −31.4 | −55.6 | 72.6 | 1 | A |
| ILE | C | 242 | −26.8 | −53.9 | 72.2 | 1 | A |
| ILE | O | 242 | −26.6 | −53.7 | 73.4 | 4 | A |
| ILE | N | 243 | −26.2 | −53.3 | 71.2 | 1 | A |
| ILE | CA | 243 | −25.3 | −52.2 | 71.5 | 1 | A |
| ILE | CB | 243 | −24.2 | −52.1 | 70.3 | 1 | A |
| ILE | CG2 | 243 | −23.5 | −50.8 | 70.4 | 1 | A |
| ILE | CG1 | 243 | −23.3 | −53.2 | 70.4 | 1 | A |
| ILE | CD1 | 243 | −22.2 | −53.2 | 69.4 | 1 | A |
| ILE | C | 243 | −26.1 | −50.9 | 71.6 | 1 | A |
| ILE | O | 243 | −27.0 | −50.7 | 70.7 | 1 | A |
| VAL | N | 244 | −25.9 | −50.1 | 72.6 | 1 | A |
| VAL | CA | 244 | −26.7 | −48.9 | 72.7 | 1 | A |
| VAL | CB | 244 | −27.3 | −48.8 | 74.1 | 1 | A |
| VAL | CG1 | 244 | −28.3 | −49.8 | 74.4 | 1 | A |
| VAL | CG2 | 244 | −26.2 | −48.8 | 75.2 | 1 | A |
| VAL | C | 244 | −25.9 | −47.6 | 72.4 | 1 | A |
| VAL | O | 244 | −26.5 | −46.5 | 72.5 | 1 | A |
| ARG | N | 245 | −24.6 | −47.7 | 72.2 | 1 | A |
| ARG | CA | 245 | −23.8 | −46.5 | 71.9 | 1 | A |
| ARG | CB | 245 | −23.8 | −45.7 | 73.2 | 1 | A |
| ARG | CG | 245 | −23.0 | −44.4 | 73.1 | 1 | A |
| ARG | CD | 245 | −22.7 | −43.9 | 74.6 | 1 | A |
| ARG | NE | 245 | −22.1 | −42.6 | 74.7 | 1 | A |
| ARG | CZ | 245 | −22.8 | −41.5 | 74.5 | 5 | A |
| ARG | NH1 | 245 | −24.1 | −41.5 | 74.2 | 6 | A |
| ARG | NH2 | 245 | −22.2 | −40.3 | 74.6 | 1 | A |
| ARG | C | 245 | −22.4 | −46.9 | 71.5 | 1 | A |
| ARG | O | 245 | −22.0 | −48.0 | 71.8 | 1 | A |
| VAL | N | 246 | −21.8 | −46.0 | 70.7 | 2 | A |
| VAL | CA | 246 | −20.4 | −46.3 | 70.3 | 1 | A |
| VAL | CB | 246 | −20.4 | −46.8 | 68.9 | 1 | A |
| VAL | CG1 | 246 | −19.0 | −46.9 | 68.4 | 1 | A |
| VAL | CG2 | 246 | −21.0 | −48.2 | 68.8 | 1 | A |
| VAL | C | 246 | −19.6 | −44.9 | 70.3 | 1 | A |
| VAL | O | 246 | −20.2 | −43.9 | 69.8 | 1 | A |
| GLU | N | 247 | −18.4 | −45.0 | 70.8 | 1 | A |
| GLU | CA | 247 | −17.6 | −43.7 | 70.8 | 1 | A |
| GLU | CB | 247 | −17.5 | −43.2 | 72.3 | 2 | A |
| GLU | CG | 247 | −18.6 | −43.5 | 73.2 | 2 | A |
| GLU | CD | 247 | −18.3 | −42.9 | 74.6 | 3 | A |
| GLU | OE1 | 247 | −17.1 | −42.9 | 75.0 | 1 | A |
| GLU | OE2 | 247 | −19.3 | −42.4 | 75.3 | 3 | A |
| GLU | C | 247 | −16.2 | −44.0 | 70.3 | 1 | A |
| GLU | O | 247 | −15.7 | −45.1 | 70.5 | 1 | A |
| ILE | N | 248 | −15.6 | −43.1 | 69.7 | 1 | A |
| ILE | CA | 248 | −14.2 | −43.2 | 69.2 | 1 | A |
| ILE | CB | 248 | −14.1 | −42.9 | 67.7 | 1 | A |
| ILE | CG2 | 248 | −12.7 | −43.2 | 67.3 | 1 | A |
| ILE | CG1 | 248 | −15.1 | −43.8 | 66.9 | 1 | A |
| ILE | CD1 | 248 | −14.9 | −45.2 | 67.1 | 1 | A |
| ILE | C | 248 | −13.4 | −42.2 | 70.0 | 1 | A |
| ILE | O | 248 | −13.5 | −41.0 | 69.7 | 1 | A |
| ASN | N | 249 | −12.7 | −42.6 | 71.0 | 1 | A |
| ASN | CA | 249 | −11.9 | −41.7 | 71.8 | 1 | A |
| ASN | CB | 249 | −10.9 | −40.9 | 71.0 | 1 | A |
| ASN | CG | 249 | −9.5 | −41.4 | 71.3 | 1 | A |
| ASN | OD1 | 249 | −9.3 | −42.5 | 71.7 | 1 | A |
| ASN | ND2 | 249 | −8.5 | −40.5 | 71.0 | 1 | A |
| ASN | C | 249 | −12.9 | −40.8 | 72.5 | 1 | A |
| ASN | O | 249 | −12.7 | −39.6 | 72.5 | 1 | A |
| GLY | N | 250 | −13.9 | −41.3 | 73.1 | 1 | A |
| GLY | CA | 250 | −14.9 | −40.5 | 73.8 | 1 | A |
| GLY | C | 250 | −15.9 | −39.8 | 73.0 | 2 | A |
| GLY | O | 250 | −17.0 | −39.4 | 73.5 | 4 | A |
| GLN | N | 251 | −15.6 | −39.6 | 71.7 | 1 | A |
| GLN | CA | 251 | −16.6 | −38.9 | 70.9 | 1 | A |
| GLN | CB | 251 | −15.8 | −38.3 | 69.7 | 4 | A |
| GLN | CG | 251 | −16.6 | −37.3 | 68.9 | 1 | A |
| GLN | CD | 251 | −15.6 | −36.1 | 68.6 | 2 | A |
| GLN | OE1 | 251 | −15.8 | −35.5 | 67.5 | 6 | A |
| GLN | NE2 | 251 | −14.7 | −35.8 | 69.5 | 3 | A |
| GLN | C | 251 | −17.7 | −39.9 | 70.4 | 1 | A |
| GLN | O | 251 | −17.4 | −40.9 | 69.7 | 1 | A |
| ASP | N | 252 | −18.9 | −39.5 | 70.7 | 1 | A |
| ASP | CA | 252 | −20.1 | −40.3 | 70.4 | 1 | A |
| ASP | CB | 252 | −21.3 | −39.7 | 71.1 | 1 | A |
| ASP | CG | 252 | −22.6 | −40.4 | 70.7 | 1 | A |
| ASP | OD1 | 252 | −22.5 | −41.5 | 70.1 | 1 | A |
| ASP | OD2 | 252 | −23.7 | −39.8 | 70.9 | 1 | A |
| ASP | C | 252 | −20.3 | −40.2 | 68.9 | 1 | A |
| ASP | O | 252 | −20.5 | −39.2 | 68.4 | 3 | A |
| LEU | N | 253 | −20.2 | −41.4 | 68.3 | 1 | A |
| LEU | CA | 253 | −20.4 | −41.6 | 66.8 | 1 | A |
| LEU | CB | 253 | −20.5 | −43.0 | 66.5 | 1 | A |
| LEU | CG | 253 | −19.7 | −43.6 | 65.3 | 1 | A |
| LEU | CD1 | 253 | −18.3 | −43.1 | 65.3 | 1 | A |
| LEU | CD2 | 253 | −19.8 | −45.1 | 65.3 | 1 | A |
| LEU | C | 253 | −21.7 | −40.9 | 66.4 | 1 | A |
| LEU | O | 253 | −21.9 | −40.5 | 65.3 | 2 | A |
| LYS | N | 254 | −22.6 | −40.7 | 67.4 | 4 | A |
| LYS | CA | 254 | −23.9 | −40.1 | 67.3 | 5 | A |
| LYS | CB | 254 | −23.6 | −38.6 | 67.1 | 3 | A |
| LYS | CG | 254 | −24.7 | −37.7 | 67.7 | 8 | A |
| LYS | CD | 254 | −24.5 | −36.2 | 67.3 | 15 | A |
| LYS | CE | 254 | −25.6 | −35.3 | 67.8 | 16 | A |
| LYS | NZ | 254 | −25.2 | −33.8 | 67.6 | 16 | A |
| LYS | C | 254 | −24.7 | −40.6 | 66.2 | 6 | A |
| LYS | O | 254 | −25.0 | −39.9 | 65.2 | 10 | A |
| MET | N | 255 | −25.0 | −41.9 | 66.3 | 4 | A |
| MET | CA | 255 | −25.9 | −42.6 | 65.3 | 2 | A |
| MET | CB | 255 | −25.1 | −43.7 | 64.6 | 1 | A |
| MET | CG | 255 | −24.5 | −43.4 | 63.3 | 1 | A |
| MET | SD | 255 | −23.2 | −44.6 | 62.8 | 3 | A |
| MET | CE | 255 | −24.2 | −45.7 | 61.9 | 3 | A |
| MET | C | 255 | −27.1 | −43.1 | 66.0 | 1 | A |
| MET | O | 255 | −27.2 | −43.1 | 67.2 | 3 | A |
| ASP | N | 256 | −28.1 | −43.5 | 65.2 | 1 | A |
| ASP | CA | 256 | −29.4 | −44.0 | 65.9 | 1 | A |
| ASP | CB | 256 | −30.4 | −44.0 | 64.8 | 1 | A |
| ASP | CG | 256 | −31.6 | −44.8 | 65.2 | 1 | A |
| ASP | OD1 | 256 | −31.5 | −46.0 | 65.2 | 1 | A |
| ASP | OD2 | 256 | −32.7 | −44.2 | 65.5 | 5 | A |
| ASP | C | 256 | −29.2 | −45.3 | 66.5 | 1 | A |
| ASP | O | 256 | −28.7 | −46.2 | 65.8 | 1 | A |
| CYS | N | 257 | −29.5 | −45.5 | 67.8 | 1 | A |
| CYS | CA | 257 | −29.4 | −46.7 | 68.5 | 1 | A |
| CYS | CB | 257 | −30.5 | −46.8 | 69.6 | 1 | A |
| CYS | SG | 257 | −30.3 | −48.2 | 71.0 | 1 | A |
| CYS | C | 257 | −29.4 | −47.9 | 67.6 | 3 | A |
| CYS | O | 257 | −28.5 | −48.8 | 67.6 | 6 | A |
| LYS | N | 258 | −30.5 | −48.0 | 66.8 | 2 | A |
| LYS | CA | 258 | −30.7 | −49.1 | 65.8 | 1 | A |
| LYS | CB | 258 | −32.0 | −48.9 | 65.1 | 1 | A |
| LYS | CG | 258 | −33.2 | −48.9 | 66.0 | 4 | A |
| LYS | CD | 258 | −34.5 | −49.4 | 65.2 | 9 | A |
| LYS | CE | 258 | −35.8 | −49.2 | 66.0 | 13 | A |
| LYS | NZ | 258 | −36.2 | −47.8 | 66.3 | 13 | A |
| LYS | C | 258 | −29.6 | −49.4 | 64.7 | 1 | A |
| LYS | O | 258 | −29.6 | −50.5 | 64.3 | 1 | A |
| GLU | N | 259 | −28.8 | −48.4 | 64.3 | 1 | A |
| GLU | CA | 259 | −27.8 | −48.7 | 63.3 | 1 | A |
| GLU | CB | 259 | −27.2 | −47.4 | 62.8 | 1 | A |
| GLU | CG | 259 | −28.0 | −46.7 | 61.7 | 1 | A |
| GLU | CD | 259 | −28.3 | −47.6 | 60.5 | 7 | A |
| GLU | OE1 | 259 | −27.5 | −48.5 | 60.3 | 5 | A |
| GLU | OE2 | 259 | −29.3 | −47.3 | 59.8 | 7 | A |
| GLU | C | 259 | −26.7 | −49.6 | 63.7 | 1 | A |
| GLU | O | 259 | −26.2 | −50.4 | 62.9 | 1 | A |
| TYR | N | 260 | −26.3 | −49.5 | 65.0 | 1 | A |
| TYR | CA | 260 | −25.2 | −50.4 | 65.5 | 1 | A |
| TYR | CB | 260 | −24.8 | −49.9 | 66.9 | 1 | A |
| TYR | CG | 260 | −24.4 | −48.4 | 67.0 | 1 | A |
| TYR | CD1 | 260 | −24.8 | −47.8 | 68.2 | 1 | A |
| TYR | CE1 | 260 | −24.5 | −46.4 | 68.3 | 1 | A |
| TYR | CD2 | 260 | −23.7 | −47.8 | 66.1 | 1 | A |
| TYR | CE2 | 260 | −23.3 | −46.4 | 66.2 | 1 | A |
| TYR | CZ | 260 | −23.7 | −45.8 | 67.4 | 1 | A |
| TYR | OH | 260 | −23.5 | −44.4 | 67.6 | 1 | A |
| TYR | C | 260 | −25.7 | −51.8 | 65.6 | 1 | A |
| TYR | O | 260 | −24.8 | −52.7 | 65.6 | 1 | A |
| ASN | N | 261 | −27.0 | −52.0 | 65.7 | 1 | A |
| ASN | CA | 261 | −27.5 | −53.4 | 65.8 | 1 | A |
| ASN | CB | 261 | −28.4 | −53.5 | 67.1 | 1 | A |
| ASN | CG | 261 | −27.7 | −53.0 | 68.3 | 1 | A |
| ASN | OD1 | 261 | −26.6 | −53.5 | 68.7 | 1 | A |
| ASN | ND2 | 261 | −28.3 | −52.0 | 69.0 | 1 | A |
| ASN | C | 261 | −28.4 | −53.8 | 64.6 | 1 | A |
| ASN | O | 261 | −29.3 | −54.6 | 64.8 | 1 | A |
| TYR | N | 262 | −28.1 | −53.2 | 63.5 | 7 | A |
| TYR | CA | 262 | −28.9 | −53.4 | 62.3 | 7 | A |
| TYR | CB | 262 | −28.4 | −52.5 | 61.1 | 6 | A |
| TYR | CG | 262 | −29.2 | −52.8 | 59.9 | 8 | A |
| TYR | CD1 | 262 | −30.5 | −53.0 | 59.8 | 8 | A |
| TYR | CE1 | 262 | −31.2 | −53.3 | 58.7 | 7 | A |
| TYR | CD2 | 262 | −28.5 | −53.0 | 58.7 | 12 | A |
| TYR | CE2 | 262 | −29.1 | −53.3 | 57.5 | 11 | A |
| TYR | CZ | 262 | −30.5 | −53.4 | 57.5 | 10 | A |
| TYR | OH | 262 | −31.1 | −53.8 | 56.4 | 12 | A |
| TYR | C | 262 | −28.9 | −54.8 | 61.8 | 7 | A |
| TYR | O | 262 | −27.9 | −55.3 | 61.1 | 4 | A |
| ASP | N | 263 | −30.0 | −55.5 | 62.0 | 8 | A |
| ASP | CA | 263 | −30.2 | −56.9 | 61.7 | 6 | A |
| ASP | CB | 263 | −29.2 | −57.4 | 60.6 | 8 | A |
| ASP | CG | 263 | −29.6 | −58.8 | 60.1 | 15 | A |
| ASP | OD1 | 263 | −30.8 | −59.2 | 60.2 | 17 | A |
| ASP | OD2 | 263 | −28.7 | −59.6 | 59.7 | 19 | A |
| ASP | C | 263 | −29.9 | −57.6 | 63.1 | 5 | A |
| ASP | O | 263 | −30.8 | −58.0 | 63.8 | 5 | A |
| LYS | N | 264 | −28.6 | −57.6 | 63.4 | 5 | A |
| LYS | CA | 264 | −28.3 | −58.3 | 64.7 | 4 | A |
| LYS | CB | 264 | −28.4 | −59.8 | 64.6 | 2 | A |
| LYS | CG | 264 | −27.5 | −60.4 | 63.5 | 1 | A |
| LYS | CD | 264 | −27.6 | −61.9 | 63.6 | 1 | A |
| LYS | CE | 264 | −26.7 | −62.5 | 62.4 | 4 | A |
| LYS | NZ | 264 | −27.4 | −62.3 | 61.1 | 7 | A |
| LYS | C | 264 | −26.9 | −57.9 | 65.2 | 1 | A |
| LYS | O | 264 | −26.0 | −57.4 | 64.4 | 1 | A |
| SER | N | 265 | −26.6 | −58.2 | 66.5 | 1 | A |
| SER | CA | 265 | −25.4 | −57.9 | 67.1 | 1 | A |
| SER | CB | 265 | −25.4 | −56.7 | 68.0 | 1 | A |
| SER | OG | 265 | −25.4 | −55.5 | 67.3 | 6 | A |
| SER | C | 265 | −25.1 | −59.2 | 67.9 | 1 | A |
| SER | O | 265 | −25.9 | −59.6 | 68.7 | 1 | A |
| ILE | N | 266 | −23.9 | −59.8 | 67.6 | 1 | A |
| ILE | CA | 266 | −23.6 | −61.1 | 68.2 | 1 | A |
| ILE | CB | 266 | −23.8 | −62.2 | 67.2 | 1 | A |
| ILE | CG2 | 266 | −25.2 | −62.2 | 66.7 | 1 | A |
| ILE | CG1 | 266 | −22.9 | −62.0 | 66.0 | 2 | A |
| ILE | CD1 | 266 | −23.0 | −63.1 | 64.9 | 1 | A |
| ILE | C | 266 | −22.1 | −61.1 | 68.7 | 1 | A |
| ILE | O | 266 | −21.3 | −60.4 | 68.3 | 1 | A |
| VAL | N | 267 | −21.9 | −62.1 | 69.7 | 2 | A |
| VAL | CA | 267 | −20.5 | −62.3 | 70.2 | 1 | A |
| VAL | CB | 267 | −20.5 | −62.3 | 71.7 | 1 | A |
| VAL | CG1 | 267 | −19.1 | −62.5 | 72.2 | 4 | A |
| VAL | CG2 | 267 | −21.1 | −61.1 | 72.3 | 2 | A |
| VAL | C | 267 | −20.1 | −63.6 | 69.6 | 2 | A |
| VAL | O | 267 | −20.5 | −64.6 | 70.1 | 4 | A |
| ASP | N | 268 | −19.3 | −63.5 | 68.5 | 2 | A |
| ASP | CA | 268 | −18.9 | −64.7 | 67.8 | 1 | A |
| ASP | CB | 268 | −19.4 | −64.6 | 66.3 | 2 | A |
| ASP | CG | 268 | −18.8 | −65.6 | 65.4 | 4 | A |
| ASP | OD1 | 268 | −18.9 | −66.8 | 65.5 | 4 | A |
| ASP | OD2 | 268 | −18.1 | −65.1 | 64.4 | 4 | A |
| ASP | C | 268 | −17.4 | −64.9 | 67.8 | 1 | A |
| ASP | O | 268 | −16.7 | −64.1 | 67.2 | 1 | A |
| SER | N | 269 | −16.9 | −66.0 | 68.4 | 1 | A |
| SER | CA | 269 | −15.5 | −66.2 | 68.5 | 1 | A |
| SER | CB | 269 | −15.1 | −67.1 | 69.7 | 1 | A |
| SER | OG | 269 | −15.6 | −68.4 | 69.5 | 1 | A |
| SER | C | 269 | −14.9 | −66.9 | 67.3 | 1 | A |
| SER | O | 269 | −13.7 | −67.1 | 67.1 | 1 | A |
| GLY | N | 270 | −15.8 | −67.1 | 66.3 | 1 | A |
| GLY | CA | 270 | −15.4 | −67.7 | 65.1 | 3 | A |
| GLY | C | 270 | −14.8 | −66.8 | 64.1 | 3 | A |
| GLY | O | 270 | −14.1 | −67.1 | 63.2 | 3 | A |
| THR | N | 271 | −15.1 | −65.5 | 64.2 | 4 | A |
| THR | CA | 271 | −14.6 | −64.4 | 63.3 | 7 | A |
| THR | CB | 271 | −15.8 | −63.4 | 62.9 | 11 | A |
| THR | OG1 | 271 | −17.0 | −64.2 | 62.6 | 14 | A |
| THR | CG2 | 271 | −15.4 | −62.7 | 61.7 | 17 | A |
| THR | C | 271 | −13.5 | −63.7 | 64.0 | 6 | A |
| THR | O | 271 | −13.5 | −63.4 | 65.2 | 5 | A |
| THR | N | 272 | −12.5 | −63.3 | 63.1 | 3 | A |
| THR | CA | 272 | −11.4 | −62.5 | 63.6 | 1 | A |
| THR | CB | 272 | −10.2 | −62.6 | 62.6 | 1 | A |
| THR | OG1 | 272 | −9.9 | −63.9 | 62.3 | 1 | A |
| THR | CG2 | 272 | −9.0 | −61.9 | 63.1 | 1 | A |
| THR | C | 272 | −11.7 | −61.1 | 63.9 | 1 | A |
| THR | O | 272 | −11.6 | −60.7 | 65.1 | 2 | A |
| ASN | N | 273 | −11.9 | −60.3 | 62.9 | 1 | A |
| ASN | CA | 273 | −12.2 | −58.9 | 63.0 | 1 | A |
| ASN | CB | 273 | −12.4 | −58.3 | 61.6 | 1 | A |
| ASN | CG | 273 | −11.1 | −58.5 | 60.8 | 2 | A |
| ASN | OD1 | 273 | −10.2 | −59.2 | 61.1 | 1 | A |
| ASN | ND2 | 273 | −11.1 | −57.8 | 59.6 | 1 | A |
| ASN | C | 273 | −13.5 | −58.5 | 63.7 | 1 | A |
| ASN | O | 273 | −14.3 | −59.3 | 64.1 | 6 | A |
| LEU | N | 274 | −13.6 | −57.2 | 64.0 | 1 | A |
| LEU | CA | 274 | −14.8 | −56.6 | 64.7 | 1 | A |
| LEU | CB | 274 | −14.5 | −55.4 | 65.5 | 1 | A |
| LEU | CG | 274 | −15.6 | −54.6 | 66.2 | 1 | A |
| LEU | CD1 | 274 | −15.2 | −53.2 | 66.3 | 1 | A |
| LEU | CD2 | 274 | −16.9 | −54.8 | 65.6 | 1 | A |
| LEU | C | 274 | −15.5 | −56.2 | 63.4 | 1 | A |
| LEU | O | 274 | −15.0 | −55.4 | 62.6 | 4 | A |
| ARG | N | 275 | −16.7 | −56.7 | 63.2 | 1 | A |
| ARG | CA | 275 | −17.5 | −56.4 | 62.0 | 1 | A |
| ARG | CB | 275 | −18.0 | −57.6 | 61.3 | 1 | A |
| ARG | CG | 275 | −16.9 | −58.4 | 60.5 | 1 | A |
| ARG | CD | 275 | −17.3 | −59.8 | 60.2 | 8 | A |
| ARG | NE | 275 | −18.5 | −60.0 | 59.4 | 16 | A |
| ARG | CZ | 275 | −18.6 | −59.6 | 58.2 | 17 | A |
| ARG | NH1 | 275 | −19.8 | −59.8 | 57.5 | 19 | A |
| ARG | NH2 | 275 | −17.6 | −59.1 | 57.5 | 20 | A |
| ARG | C | 275 | −18.6 | −55.4 | 62.3 | 1 | A |
| ARG | O | 275 | −19.4 | −55.6 | 63.2 | 2 | A |
| LEU | N | 276 | −18.7 | −54.3 | 61.6 | 1 | A |
| LEU | CA | 276 | −19.7 | −53.3 | 61.8 | 1 | A |
| LEU | CB | 276 | −19.0 | −52.0 | 62.1 | 1 | A |
| LEU | CG | 276 | −18.1 | −51.9 | 63.4 | 1 | A |
| LEU | CD1 | 276 | −17.6 | −50.5 | 63.5 | 1 | A |
| LEU | CD2 | 276 | −18.9 | −52.2 | 64.6 | 1 | A |
| LEU | C | 276 | −20.5 | −53.1 | 60.6 | 1 | A |
| LEU | O | 276 | −20.1 | −53.2 | 59.5 | 3 | A |
| PRO | N | 277 | −21.8 | −52.8 | 60.8 | 3 | A |
| PRO | CD | 277 | −22.6 | −53.0 | 62.0 | 4 | A |
| PRO | CA | 277 | −22.7 | −52.6 | 59.6 | 6 | A |
| PRO | CB | 277 | −24.0 | −52.2 | 60.3 | 5 | A |
| PRO | CG | 277 | −24.0 | −53.1 | 61.5 | 1 | A |
| PRO | C | 277 | −22.2 | −51.5 | 58.7 | 8 | A |
| PRO | O | 277 | −21.6 | −50.6 | 59.1 | 10 | A |
| LYS | N | 278 | −22.4 | −51.7 | 57.4 | 11 | A |
| LYS | CA | 278 | −21.9 | −50.8 | 56.4 | 13 | A |
| LYS | CB | 278 | −22.5 | −51.2 | 55.0 | 18 | A |
| LYS | CG | 278 | −21.9 | −50.5 | 53.9 | 22 | A |
| LYS | CD | 278 | −20.4 | −50.6 | 53.9 | 23 | A |
| LYS | CE | 278 | −19.7 | −49.7 | 52.9 | 26 | A |
| LYS | NZ | 278 | −18.2 | −49.6 | 53.0 | 21 | A |
| LYS | C | 278 | −22.1 | −49.4 | 56.7 | 10 | A |
| LYS | O | 278 | −21.4 | −48.5 | 56.1 | 13 | A |
| LYS | N | 279 | −23.0 | −49.0 | 57.5 | 11 | A |
| LYS | CA | 279 | −23.2 | −47.6 | 57.8 | 12 | A |
| LYS | CB | 279 | −24.7 | −47.4 | 58.3 | 18 | A |
| LYS | CG | 279 | −25.7 | −47.7 | 57.2 | 25 | A |
| LYS | CD | 279 | −26.6 | −48.9 | 57.6 | 34 | A |
| LYS | CE | 279 | −27.9 | −49.0 | 56.7 | 38 | A |
| LYS | NZ | 279 | −28.9 | −50.0 | 57.3 | 38 | A |
| LYS | C | 279 | −22.3 | −47.2 | 59.0 | 8 | A |
| LYS | O | 279 | −21.7 | −46.1 | 59.0 | 4 | A |
| VAL | N | 280 | −22.2 | −48.1 | 60.0 | 7 | A |
| VAL | CA | 280 | −21.4 | −47.9 | 61.2 | 3 | A |
| VAL | CB | 280 | −21.8 | −48.9 | 62.2 | 1 | A |
| VAL | CG1 | 280 | −21.0 | −48.6 | 63.5 | 2 | A |
| VAL | CG2 | 280 | −23.2 | −48.7 | 62.6 | 1 | A |
| VAL | C | 280 | −19.9 | −48.0 | 60.9 | 3 | A |
| VAL | O | 280 | −19.1 | −47.4 | 61.6 | 3 | A |
| PHE | N | 281 | −19.5 | −48.7 | 59.9 | 2 | A |
| PHE | CA | 281 | −18.1 | −48.9 | 59.5 | 1 | A |
| PHE | CB | 281 | −17.9 | −50.0 | 58.6 | 1 | A |
| PHE | CG | 281 | −16.5 | −50.1 | 58.0 | 1 | A |
| PHE | CD1 | 281 | −15.5 | −50.7 | 58.8 | 5 | A |
| PHE | CD2 | 281 | −16.2 | −49.7 | 56.8 | 1 | A |
| PHE | CE1 | 281 | −14.3 | −50.9 | 58.3 | 3 | A |
| PHE | CE2 | 281 | −14.9 | −49.8 | 56.3 | 4 | A |
| PHE | CZ | 281 | −13.9 | −50.4 | 57.1 | 5 | A |
| PHE | C | 281 | −17.6 | −47.6 | 58.9 | 1 | A |
| PHE | O | 281 | −16.4 | −47.2 | 59.2 | 1 | A |
| GLU | N | 282 | −18.4 | −47.0 | 58.1 | 1 | A |
| GLU | CA | 282 | −18.0 | −45.7 | 57.4 | 5 | A |
| GLU | CB | 282 | −19.1 | −45.3 | 56.4 | 9 | A |
| GLU | CG | 282 | −19.5 | −46.4 | 55.4 | 15 | A |
| GLU | CD | 282 | −18.8 | −46.2 | 54.1 | 19 | A |
| GLU | OE1 | 282 | −19.3 | −45.3 | 53.3 | 23 | A |
| GLU | OE2 | 282 | −17.9 | −47.0 | 53.7 | 21 | A |
| GLU | C | 282 | −17.7 | −44.6 | 58.4 | 7 | A |
| GLU | O | 282 | −16.8 | −43.8 | 58.2 | 11 | A |
| ALA | N | 283 | −18.6 | −44.5 | 59.4 | 8 | A |
| ALA | CA | 283 | −18.5 | −43.5 | 60.4 | 6 | A |
| ALA | CB | 283 | −19.9 | −43.3 | 61.1 | 7 | A |
| ALA | C | 283 | −17.4 | −43.7 | 61.4 | 3 | A |
| ALA | O | 283 | −16.8 | −42.8 | 61.9 | 7 | A |
| ALA | N | 284 | −17.2 | −45.0 | 61.8 | 1 | A |
| ALA | CA | 284 | −16.2 | −45.3 | 62.7 | 1 | A |
| ALA | CB | 284 | −16.4 | −46.7 | 63.2 | 1 | A |
| ALA | C | 284 | −14.8 | −45.1 | 62.1 | 1 | A |
| ALA | O | 284 | −13.9 | −44.5 | 62.7 | 1 | A |
| VAL | N | 285 | −14.6 | −45.6 | 60.9 | 1 | A |
| VAL | CA | 285 | −13.4 | −45.5 | 60.3 | 1 | A |
| VAL | CB | 285 | −13.3 | −46.2 | 58.9 | 1 | A |
| VAL | CG1 | 285 | −12.1 | −45.8 | 58.1 | 1 | A |
| VAL | CG2 | 285 | −13.4 | −47.7 | 59.1 | 2 | A |
| VAL | C | 285 | −13.1 | −44.0 | 60.0 | 1 | A |
| VAL | O | 285 | −12.0 | −43.5 | 60.1 | 1 | A |
| LYS | N | 286 | −14.2 | −43.3 | 59.7 | 2 | A |
| LYS | CA | 286 | −14.0 | −41.9 | 59.4 | 3 | A |
| LYS | CB | 286 | −15.4 | −41.3 | 59.1 | 3 | A |
| LYS | CG | 286 | −15.4 | −39.8 | 58.5 | 15 | A |
| LYS | CD | 286 | −14.2 | −39.7 | 57.5 | 26 | A |
| LYS | CE | 286 | −14.2 | −40.7 | 56.3 | 27 | A |
| LYS | NZ | 286 | −12.9 | −40.8 | 55.6 | 21 | A |
| LYS | C | 286 | −13.4 | −41.2 | 60.6 | 1 | A |
| LYS | O | 286 | −12.5 | −40.3 | 60.5 | 1 | A |
| SER | N | 287 | −13.9 | −41.6 | 61.8 | 1 | A |
| SER | CA | 287 | −13.4 | −41.0 | 63.0 | 1 | A |
| SER | CB | 287 | −14.4 | −41.3 | 64.1 | 1 | A |
| SER | OG | 287 | −13.9 | −40.9 | 65.4 | 1 | A |
| SER | C | 287 | −12.0 | −41.5 | 63.4 | 1 | A |
| SER | O | 287 | −11.2 | −40.8 | 63.9 | 1 | A |
| ILE | N | 288 | −11.8 | −42.8 | 63.2 | 1 | A |
| ILE | CA | 288 | −10.5 | −43.4 | 63.5 | 1 | A |
| ILE | CB | 288 | −10.6 | −45.0 | 63.4 | 1 | A |
| ILE | CG2 | 288 | −9.2 | −45.6 | 63.7 | 1 | A |
| ILE | CG1 | 288 | −11.7 | −45.5 | 64.3 | 1 | A |
| ILE | CD1 | 288 | −11.9 | −47.0 | 64.1 | 1 | A |
| ILE | C | 288 | −9.5 | −42.8 | 62.7 | 1 | A |
| ILE | O | 288 | −8.4 | −42.5 | 63.1 | 1 | A |
| LYS | N | 289 | −9.8 | −42.7 | 61.4 | 3 | A |
| LYS | CA | 289 | −8.9 | −42.2 | 60.4 | 3 | A |
| LYS | CB | 289 | −9.6 | −42.1 | 59.0 | 1 | A |
| LYS | CG | 289 | −8.6 | −42.0 | 57.8 | 5 | A |
| LYS | CD | 289 | −9.3 | −42.2 | 56.5 | 1 | A |
| LYS | CE | 289 | −10.0 | −43.5 | 56.3 | 2 | A |
| LYS | NZ | 289 | −10.6 | −43.8 | 55.0 | 2 | A |
| LYS | C | 289 | −8.5 | −40.8 | 60.9 | 4 | A |
| LYS | O | 289 | −7.3 | −40.4 | 60.9 | 7 | A |
| ALA | N | 290 | −9.5 | −40.0 | 61.3 | 5 | A |
| ALA | CA | 290 | −9.3 | −38.7 | 61.8 | 2 | A |
| ALA | CB | 290 | −10.6 | −38.0 | 62.1 | 1 | A |
| ALA | C | 290 | −8.4 | −38.6 | 63.1 | 3 | A |
| ALA | O | 290 | −7.4 | −37.8 | 63.1 | 4 | A |
| ALA | N | 291 | −8.7 | −39.4 | 64.0 | 2 | A |
| ALA | CA | 291 | −8.0 | −39.5 | 65.3 | 1 | A |
| ALA | CB | 291 | −8.6 | −40.4 | 66.3 | 1 | A |
| ALA | C | 291 | −6.5 | −39.8 | 65.1 | 1 | A |
| ALA | O | 291 | −5.7 | −39.7 | 66.0 | 1 | A |
| SER | N | 292 | −6.1 | −40.3 | 63.9 | 1 | A |
| SER | CA | 292 | −4.8 | −40.8 | 63.7 | 1 | A |
| SER | CB | 292 | −4.7 | −42.2 | 63.5 | 1 | A |
| SER | OG | 292 | −5.9 | −42.6 | 62.7 | 1 | A |
| SER | C | 292 | −4.1 | −40.0 | 62.5 | 4 | A |
| SER | O | 292 | −3.4 | −40.6 | 61.8 | 7 | A |
| SER | N | 293 | −4.4 | −38.7 | 62.4 | 5 | A |
| SER | CA | 293 | −3.9 | −37.9 | 61.3 | 6 | A |
| SER | CB | 293 | −4.6 | −36.6 | 61.3 | 6 | A |
| SER | OG | 293 | −6.0 | −36.7 | 61.3 | 10 | A |
| SER | C | 293 | −2.4 | −37.7 | 61.3 | 7 | A |
| SER | O | 293 | −1.8 | −37.6 | 60.2 | 7 | A |
| THR | N | 294 | −1.8 | −37.6 | 62.5 | 8 | A |
| THR | CA | 294 | −0.4 | −37.4 | 62.6 | 7 | A |
| THR | CB | 294 | 0.1 | −37.9 | 64.0 | 9 | A |
| THR | OG1 | 294 | −0.8 | −37.5 | 65.0 | 10 | A |
| THR | CG2 | 294 | 1.5 | −37.2 | 64.3 | 8 | A |
| THR | C | 294 | 0.3 | −38.2 | 61.5 | 6 | A |
| THR | O | 294 | 1.2 | −37.7 | 60.8 | 9 | A |
| GLU | N | 295 | −0.1 | −39.5 | 61.4 | 6 | A |
| GLU | CA | 295 | 0.5 | −40.3 | 60.3 | 7 | A |
| GLU | CB | 295 | 1.0 | −41.6 | 61.0 | 9 | A |
| GLU | CG | 295 | 2.3 | −41.5 | 61.8 | 9 | A |
| GLU | CD | 295 | 2.8 | −42.7 | 62.4 | 6 | A |
| GLU | OE1 | 295 | 2.8 | −43.8 | 61.8 | 8 | A |
| GLU | OE2 | 295 | 3.1 | −42.7 | 63.6 | 12 | A |
| GLU | C | 295 | −0.5 | −40.6 | 59.3 | 9 | A |
| GLU | O | 295 | −1.7 | −40.4 | 59.5 | 11 | A |
| LYS | N | 296 | 0.0 | −41.1 | 58.1 | 12 | A |
| LYS | CA | 296 | −0.9 | −41.4 | 57.0 | 11 | A |
| LYS | CB | 296 | −0.7 | −40.4 | 55.8 | 15 | A |
| LYS | CG | 296 | −1.5 | −39.1 | 55.9 | 19 | A |
| LYS | CD | 296 | −3.0 | −39.3 | 55.4 | 22 | A |
| LYS | CE | 296 | −3.7 | −38.0 | 55.2 | 21 | A |
| LYS | NZ | 296 | −3.9 | −37.2 | 56.4 | 16 | A |
| LYS | C | 296 | −0.7 | −42.8 | 56.5 | 10 | A |
| LYS | O | 296 | 0.5 | −43.2 | 56.6 | 11 | A |
| PHE | N | 297 | −1.7 | −43.5 | 56.1 | 8 | A |
| PHE | CA | 297 | −1.5 | −44.9 | 55.7 | 10 | A |
| PHE | CB | 297 | −2.2 | −45.9 | 56.7 | 10 | A |
| PHE | CG | 297 | −2.2 | −45.4 | 58.1 | 10 | A |
| PHE | CD1 | 297 | −3.1 | −44.4 | 58.5 | 14 | A |
| PHE | CD2 | 297 | −1.4 | −45.9 | 59.1 | 10 | A |
| PHE | CE1 | 297 | −3.2 | −43.9 | 59.8 | 12 | A |
| PHE | CE2 | 297 | −1.5 | −45.5 | 60.4 | 13 | A |
| PHE | CZ | 297 | −2.4 | −44.5 | 60.8 | 10 | A |
| PHE | C | 297 | −2.2 | −45.0 | 54.3 | 12 | A |
| PHE | O | 297 | −3.1 | −44.3 | 54.0 | 13 | A |
| PRO | N | 298 | −1.7 | −46.0 | 53.5 | 12 | A |
| PRO | CD | 298 | −0.6 | −46.9 | 53.7 | 8 | A |
| PRO | CA | 298 | −2.3 | −46.2 | 52.2 | 15 | A |
| PRO | CB | 298 | −1.5 | −47.4 | 51.6 | 11 | A |
| PRO | CG | 298 | −0.9 | −48.1 | 52.8 | 11 | A |
| PRO | C | 298 | −3.8 | −46.5 | 52.3 | 19 | A |
| PRO | O | 298 | −4.2 | −47.2 | 53.3 | 18 | A |
| ASP | N | 299 | −4.6 | −46.1 | 51.3 | 25 | A |
| ASP | CA | 299 | −6.1 | −46.2 | 51.3 | 28 | A |
| ASP | CB | 299 | −6.6 | −45.7 | 50.0 | 31 | A |
| ASP | CG | 299 | −8.1 | −45.4 | 50.1 | 35 | A |
| ASP | OD1 | 299 | −8.5 | −44.5 | 50.9 | 36 | A |
| ASP | OD2 | 299 | −8.9 | −46.1 | 49.4 | 37 | A |
| ASP | C | 299 | −6.5 | −47.7 | 51.5 | 26 | A |
| ASP | O | 299 | −7.6 | −47.9 | 52.1 | 20 | A |
| GLY | N | 300 | −5.7 | −48.7 | 51.1 | 21 | A |
| GLY | CA | 300 | −6.1 | −50.0 | 51.3 | 16 | A |
| GLY | C | 300 | −6.0 | −50.5 | 52.7 | 13 | A |
| GLY | O | 300 | −6.6 | −51.5 | 53.1 | 12 | A |
| PHE | N | 301 | −5.2 | −49.8 | 53.5 | 10 | A |
| PHE | CA | 301 | −5.0 | −50.1 | 54.9 | 6 | A |
| PHE | CB | 301 | −4.1 | −49.0 | 55.6 | 1 | A |
| PHE | CG | 301 | −4.1 | −49.2 | 57.1 | 1 | A |
| PHE | CD1 | 301 | −3.6 | −50.4 | 57.6 | 1 | A |
| PHE | CD2 | 301 | −4.5 | −48.2 | 57.9 | 1 | A |
| PHE | CE1 | 301 | −3.6 | −50.5 | 59.0 | 1 | A |
| PHE | CE2 | 301 | −4.5 | −48.3 | 59.3 | 1 | A |
| PHE | CZ | 301 | −4.0 | −49.5 | 59.9 | 1 | A |
| PHE | C | 301 | −6.3 | −50.2 | 55.6 | 6 | A |
| PHE | O | 301 | −6.6 | −51.2 | 56.1 | 9 | A |
| TRP | N | 302 | −7.1 | −49.1 | 55.5 | 3 | A |
| TRP | CA | 302 | −8.4 | −49.0 | 56.2 | 3 | A |
| TRP | CB | 302 | −8.9 | −47.6 | 56.2 | 4 | A |
| TRP | CG | 302 | −8.1 | −46.6 | 56.9 | 1 | A |
| TRP | CD2 | 302 | −7.9 | −46.6 | 58.3 | 1 | A |
| TRP | CE2 | 302 | −7.0 | −45.5 | 58.6 | 1 | A |
| TRP | CE3 | 302 | −8.3 | −47.3 | 59.4 | 3 | A |
| TRP | CD1 | 302 | −7.4 | −45.6 | 56.4 | 1 | A |
| TRP | NE1 | 302 | −6.8 | −44.9 | 57.4 | 1 | A |
| TRP | CZ2 | 302 | −6.6 | −45.1 | 59.9 | 1 | A |
| TRP | CZ3 | 302 | −7.9 | −47.0 | 60.7 | 1 | A |
| TRP | CH2 | 302 | −7.1 | −45.9 | 60.9 | 1 | A |
| TRP | C | 302 | −9.4 | −50.0 | 55.6 | 5 | A |
| TRP | O | 302 | −10.5 | −50.1 | 56.1 | 7 | A |
| LEU | N | 303 | −9.0 | −50.8 | 54.6 | 9 | A |
| LEU | CA | 303 | −9.9 | −51.8 | 54.1 | 10 | A |
| LEU | CB | 303 | −10.1 | −51.6 | 52.6 | 9 | A |
| LEU | CG | 303 | −10.7 | −50.3 | 52.1 | 11 | A |
| LEU | CD1 | 303 | −11.0 | −50.4 | 50.6 | 15 | A |
| LEU | CD2 | 303 | −12.0 | −50.0 | 52.9 | 11 | A |
| LEU | C | 303 | −9.4 | −53.3 | 54.2 | 9 | A |
| LEU | O | 303 | −9.9 | −54.2 | 53.6 | 10 | A |
| GLY | N | 304 | −8.4 | −53.5 | 55.1 | 10 | A |
| GLY | CA | 304 | −7.9 | −54.8 | 55.3 | 12 | A |
| GLY | C | 304 | −7.1 | −55.4 | 54.2 | 13 | A |
| GLY | O | 304 | −6.8 | −56.6 | 54.2 | 13 | A |
| GLU | N | 305 | −6.9 | −54.6 | 53.1 | 14 | A |
| GLU | CA | 305 | −6.2 | −55.0 | 51.9 | 13 | A |
| GLU | CB | 305 | −6.8 | −54.3 | 50.7 | 16 | A |
| GLU | CG | 305 | −8.1 | −54.9 | 50.2 | 19 | A |
| GLU | CD | 305 | −8.8 | −54.0 | 49.2 | 23 | A |
| GLU | OE1 | 305 | −9.8 | −54.4 | 48.5 | 29 | A |
| GLU | OE2 | 305 | −8.3 | −52.8 | 49.0 | 23 | A |
| GLU | C | 305 | −4.7 | −54.9 | 52.0 | 11 | A |
| GLU | O | 305 | −4.0 | −55.5 | 51.2 | 14 | A |
| GLN | N | 306 | −4.2 | −54.1 | 52.9 | 8 | A |
| GLN | CA | 306 | −2.8 | −53.9 | 53.0 | 10 | A |
| GLN | CB | 306 | −2.3 | −53.0 | 51.9 | 11 | A |
| GLN | CG | 306 | −2.9 | −51.6 | 51.9 | 20 | A |
| GLN | CD | 306 | −2.7 | −50.9 | 50.5 | 27 | A |
| GLN | OE1 | 306 | −3.1 | −49.7 | 50.4 | 22 | A |
| GLN | NE2 | 306 | −2.2 | −51.6 | 49.5 | 27 | A |
| GLN | C | 306 | −2.3 | −53.5 | 54.3 | 8 | A |
| GLN | O | 306 | −2.8 | −52.5 | 54.9 | 9 | A |
| LEU | N | 307 | −1.3 | −54.2 | 54.9 | 6 | A |
| LEU | CA | 307 | −0.8 | −53.9 | 56.2 | 3 | A |
| LEU | CB | 307 | −0.2 | −55.1 | 56.8 | 1 | A |
| LEU | CG | 307 | 0.6 | −56.1 | 55.9 | 2 | A |
| LEU | CD1 | 307 | 1.5 | −55.3 | 54.9 | 9 | A |
| LEU | CD2 | 307 | 1.4 | −57.0 | 56.7 | 2 | A |
| LEU | C | 307 | 0.1 | −52.7 | 56.4 | 3 | A |
| LEU | O | 307 | 0.8 | −52.3 | 55.5 | 3 | A |
| VAL | N | 308 | 0.1 | −52.1 | 57.6 | 3 | A |
| VAL | CA | 308 | 1.0 | −51.0 | 58.0 | 3 | A |
| VAL | CB | 308 | 0.1 | −50.0 | 58.8 | 1 | A |
| VAL | CG1 | 308 | 1.1 | −49.1 | 59.5 | 2 | A |
| VAL | CG2 | 308 | −0.7 | −49.2 | 57.9 | 8 | A |
| VAL | C | 308 | 2.1 | −51.5 | 58.8 | 3 | A |
| VAL | O | 308 | 1.9 | −52.1 | 59.8 | 3 | A |
| CYS | N | 309 | 3.3 | −51.1 | 58.5 | 3 | A |
| CYS | CA | 309 | 4.5 | −51.5 | 59.3 | 3 | A |
| CYS | C | 309 | 5.2 | −50.4 | 60.0 | 3 | A |
| CYS | O | 309 | 5.2 | −49.2 | 59.5 | 2 | A |
| CYS | CB | 309 | 5.5 | −52.3 | 58.5 | 5 | A |
| CYS | SG | 309 | 4.9 | −53.7 | 57.6 | 4 | A |
| TRP | N | 310 | 5.9 | −50.6 | 61.1 | 2 | A |
| TRP | CA | 310 | 6.7 | −49.7 | 61.8 | 1 | A |
| TRP | CB | 310 | 5.9 | −49.2 | 63.1 | 1 | A |
| TRP | CG | 310 | 4.9 | −48.1 | 62.8 | 1 | A |
| TRP | CD2 | 310 | 3.5 | −48.2 | 62.8 | 1 | A |
| TRP | CE2 | 310 | 3.0 | −46.9 | 62.6 | 1 | A |
| TRP | CE3 | 310 | 2.6 | −49.3 | 63.0 | 1 | A |
| TRP | CD1 | 310 | 5.2 | −46.8 | 62.6 | 1 | A |
| TRP | NE1 | 310 | 4.1 | −46.1 | 62.4 | 1 | A |
| TRP | CZ2 | 310 | 1.6 | −46.7 | 62.5 | 1 | A |
| TRP | CZ3 | 310 | 1.3 | −49.0 | 63.0 | 1 | A |
| TRP | CH2 | 310 | 0.8 | −47.7 | 62.7 | 1 | A |
| TRP | C | 310 | 7.9 | −50.4 | 62.2 | 3 | A |
| TRP | O | 310 | 7.9 | −51.7 | 62.2 | 4 | A |
| GLN | N | 311 | 8.9 | −49.7 | 62.7 | 3 | A |
| GLN | CA | 311 | 10.2 | −50.3 | 63.1 | 6 | A |
| GLN | CB | 311 | 11.2 | −49.2 | 63.3 | 13 | A |
| GLN | CG | 311 | 11.1 | −48.1 | 62.2 | 26 | A |
| GLN | CD | 311 | 9.7 | −47.4 | 62.1 | 29 | A |
| GLN | OE1 | 311 | 9.3 | −46.7 | 62.9 | 31 | A |
| GLN | NE2 | 311 | 9.0 | −47.7 | 61.0 | 29 | A |
| GLN | C | 311 | 9.9 | −51.0 | 64.5 | 3 | A |
| GLN | O | 311 | 9.4 | −50.4 | 65.4 | 6 | A |
| ALA | N | 312 | 10.3 | −52.2 | 64.5 | 1 | A |
| ALA | CA | 312 | 10.1 | −53.0 | 65.8 | 4 | A |
| ALA | CB | 312 | 11.2 | −54.1 | 65.8 | 7 | A |
| ALA | C | 312 | 10.1 | −52.2 | 67.0 | 2 | A |
| ALA | O | 312 | 11.0 | −51.4 | 67.3 | 3 | A |
| GLY | N | 313 | 9.2 | −52.5 | 67.9 | 5 | A |
| GLY | CA | 313 | 9.1 | −51.8 | 69.2 | 5 | A |
| GLY | C | 313 | 8.9 | −50.3 | 69.2 | 6 | A |
| GLY | O | 313 | 9.4 | −49.6 | 70.0 | 4 | A |
| THR | N | 314 | 8.1 | −49.8 | 68.2 | 7 | A |
| THR | CA | 314 | 7.9 | −48.4 | 68.1 | 5 | A |
| THR | CB | 314 | 8.9 | −47.7 | 67.1 | 2 | A |
| THR | OG1 | 314 | 8.4 | −47.8 | 65.7 | 1 | A |
| THR | CG2 | 314 | 10.3 | −48.3 | 67.2 | 3 | A |
| THR | C | 314 | 6.5 | −48.0 | 67.6 | 3 | A |
| THR | O | 314 | 6.2 | −46.9 | 67.3 | 3 | A |
| THR | N | 315 | 5.6 | −49.0 | 67.6 | 2 | A |
| THR | CA | 315 | 4.2 | −48.8 | 67.1 | 1 | A |
| THR | CB | 315 | 3.4 | −50.0 | 67.2 | 1 | A |
| THR | OG1 | 315 | 4.1 | −51.1 | 66.6 | 4 | A |
| THR | CG2 | 315 | 2.1 | −49.8 | 66.4 | 2 | A |
| THR | C | 315 | 3.6 | −47.7 | 68.0 | 1 | A |
| THR | O | 315 | 3.4 | −48.0 | 69.2 | 1 | A |
| PRO | N | 316 | 3.2 | −46.6 | 67.4 | 1 | A |
| PRO | CD | 316 | 3.3 | −46.4 | 66.0 | 2 | A |
| PRO | CA | 316 | 2.6 | −45.4 | 68.1 | 1 | A |
| PRO | CB | 316 | 2.7 | −44.3 | 67.0 | 1 | A |
| PRO | CG | 316 | 2.4 | −45.1 | 65.8 | 2 | A |
| PRO | C | 316 | 1.1 | −45.7 | 68.5 | 3 | A |
| PRO | O | 316 | 0.2 | −45.0 | 68.0 | 4 | A |
| TRP | N | 317 | 0.9 | −46.6 | 69.4 | 1 | A |
| TRP | CA | 317 | −0.5 | −46.9 | 69.8 | 1 | A |
| TRP | CB | 317 | −0.5 | −47.9 | 71.0 | 1 | A |
| TRP | CG | 317 | 0.3 | −49.1 | 70.7 | 1 | A |
| TRP | CD2 | 317 | 0.0 | −50.1 | 69.8 | 1 | A |
| TRP | CE2 | 317 | 1.1 | −51.0 | 69.8 | 1 | A |
| TRP | CE3 | 317 | −1.1 | −50.4 | 69.0 | 1 | A |
| TRP | CD1 | 317 | 1.6 | −49.4 | 71.2 | 1 | A |
| TRP | NE1 | 317 | 2.0 | −50.5 | 70.7 | 1 | A |
| TRP | CZ2 | 317 | 1.1 | −52.2 | 69.1 | 1 | A |
| TRP | CZ3 | 317 | −1.1 | −51.6 | 68.3 | 1 | A |
| TRP | CH2 | 317 | 0.0 | −52.4 | 68.3 | 1 | A |
| TRP | C | 317 | −1.1 | −45.6 | 70.3 | 1 | A |
| TRP | O | 317 | −2.3 | −45.4 | 70.1 | 1 | A |
| ASN | N | 318 | −0.3 | −44.7 | 70.9 | 4 | A |
| ASN | CA | 318 | −0.9 | −43.5 | 71.4 | 5 | A |
| ASN | CB | 318 | 0.3 | −42.6 | 72.1 | 9 | A |
| ASN | CG | 318 | 0.8 | −41.6 | 71.2 | 12 | A |
| ASN | OD1 | 318 | 0.1 | −40.6 | 70.8 | 18 | A |
| ASN | ND2 | 318 | 2.1 | −41.7 | 70.8 | 18 | A |
| ASN | C | 318 | −1.7 | −42.7 | 70.4 | 4 | A |
| ASN | O | 318 | −2.7 | −42.2 | 70.7 | 6 | A |
| ILE | N | 319 | −1.2 | −42.6 | 69.1 | 2 | A |
| ILE | CA | 319 | −2.0 | −41.8 | 68.2 | 1 | A |
| ILE | CB | 319 | −1.3 | −41.7 | 66.8 | 1 | A |
| ILE | CG2 | 319 | 0.1 | −41.2 | 67.1 | 1 | A |
| ILE | CG1 | 319 | −1.2 | −43.0 | 66.1 | 7 | A |
| ILE | CD1 | 319 | −0.6 | −42.9 | 64.7 | 11 | A |
| ILE | C | 319 | −3.4 | −42.5 | 67.9 | 1 | A |
| ILE | O | 319 | −4.4 | −41.7 | 67.8 | 1 | A |
| PHE | N | 320 | −3.5 | −43.8 | 67.8 | 1 | A |
| PHE | CA | 320 | −4.7 | −44.5 | 67.5 | 1 | A |
| PHE | CB | 320 | −4.4 | −46.0 | 67.2 | 1 | A |
| PHE | CG | 320 | −3.8 | −46.2 | 65.9 | 1 | A |
| PHE | CD1 | 320 | −4.6 | −46.3 | 64.8 | 1 | A |
| PHE | CD2 | 320 | −2.4 | −46.3 | 65.8 | 1 | A |
| PHE | CE1 | 320 | −4.0 | −46.5 | 63.5 | 1 | A |
| PHE | CE2 | 320 | −1.9 | −46.5 | 64.5 | 1 | A |
| PHE | CZ | 320 | −2.7 | −46.6 | 63.4 | 1 | A |
| PHE | C | 320 | −5.7 | −44.4 | 68.8 | 1 | A |
| PHE | O | 320 | −5.2 | −44.5 | 69.9 | 1 | A |
| PRO | N | 321 | −7.0 | −44.2 | 68.5 | 1 | A |
| PRO | CD | 321 | −7.6 | −44.1 | 67.2 | 1 | A |
| PRO | CA | 321 | −8.0 | −44.0 | 69.5 | 1 | A |
| PRO | CB | 321 | −9.0 | −43.2 | 68.8 | 1 | A |
| PRO | CG | 321 | −9.1 | −43.9 | 67.5 | 1 | A |
| PRO | C | 321 | −8.6 | −45.3 | 70.1 | 1 | A |
| PRO | O | 321 | −8.5 | −46.3 | 69.4 | 1 | A |
| VAL | N | 322 | −9.1 | −45.3 | 71.3 | 1 | A |
| VAL | CA | 322 | −9.7 | −46.5 | 71.8 | 1 | A |
| VAL | CB | 322 | −9.8 | −46.5 | 73.4 | 1 | A |
| VAL | CG1 | 322 | −8.4 | −46.4 | 73.9 | 2 | A |
| VAL | CG2 | 322 | −10.6 | −45.3 | 73.9 | 3 | A |
| VAL | C | 322 | −11.2 | −46.5 | 71.3 | 1 | A |
| VAL | O | 322 | −11.7 | −45.4 | 71.1 | 1 | A |
| ILE | N | 323 | −11.7 | −47.6 | 71.2 | 1 | A |
| ILE | CA | 323 | −13.1 | −47.8 | 70.7 | 1 | A |
| ILE | CB | 323 | −13.2 | −48.8 | 69.5 | 1 | A |
| ILE | CG2 | 323 | −14.6 | −48.9 | 69.0 | 1 | A |
| ILE | CG1 | 323 | −12.3 | −48.3 | 68.4 | 1 | A |
| ILE | CD1 | 323 | −12.2 | −49.3 | 67.3 | 1 | A |
| ILE | C | 323 | −13.9 | −48.3 | 71.9 | 1 | A |
| ILE | O | 323 | −13.6 | −49.3 | 72.5 | 3 | A |
| SER | N | 324 | −15.0 | −47.6 | 72.2 | 1 | A |
| SER | CA | 324 | −15.9 | −48.1 | 73.2 | 1 | A |
| SER | CB | 324 | −16.1 | −47.0 | 74.3 | 1 | A |
| SER | OG | 324 | −14.9 | −46.7 | 75.0 | 1 | A |
| SER | C | 324 | −17.2 | −48.5 | 72.7 | 1 | A |
| SER | O | 324 | −17.8 | −47.8 | 71.9 | 1 | A |
| LEU | N | 325 | −17.7 | −49.6 | 73.2 | 1 | A |
| LEU | CA | 325 | −19.0 | −50.1 | 72.8 | 1 | A |
| LEU | CB | 325 | −18.9 | −51.5 | 72.2 | 1 | A |
| LEU | CG | 325 | −18.0 | −51.6 | 70.9 | 1 | A |
| LEU | CD1 | 325 | −18.1 | −53.1 | 70.5 | 1 | A |
| LEU | CD2 | 325 | −18.5 | −50.7 | 69.9 | 1 | A |
| LEU | C | 325 | −19.9 | −50.2 | 74.1 | 1 | A |
| LEU | O | 325 | −19.4 | −50.8 | 75.0 | 1 | A |
| TYR | N | 326 | −21.0 | −49.6 | 74.1 | 1 | A |
| TYR | CA | 326 | −21.9 | −49.6 | 75.2 | 1 | A |
| TYR | CB | 326 | −22.7 | −48.3 | 75.4 | 1 | A |
| TYR | CG | 326 | −21.8 | −47.2 | 76.0 | 1 | A |
| TYR | CD1 | 326 | −20.6 | −46.9 | 75.4 | 3 | A |
| TYR | CE1 | 326 | −19.8 | −45.8 | 75.9 | 4 | A |
| TYR | CD2 | 326 | −22.2 | −46.4 | 77.0 | 4 | A |
| TYR | CE2 | 326 | −21.5 | −45.3 | 77.5 | 2 | A |
| TYR | CZ | 326 | −20.3 | −45.0 | 76.9 | 3 | A |
| TYR | OH | 326 | −19.5 | −43.9 | 77.4 | 5 | A |
| TYR | C | 326 | −23.0 | −50.7 | 75.0 | 1 | A |
| TYR | O | 326 | −23.7 | −50.7 | 74.0 | 1 | A |
| LEU | N | 327 | −23.0 | −51.7 | 75.9 | 1 | A |
| LEU | CA | 327 | −24.0 | −52.8 | 75.9 | 1 | A |
| LEU | CB | 327 | −23.3 | −54.1 | 76.2 | 1 | A |
| LEU | CG | 327 | −22.0 | −54.3 | 75.4 | 1 | A |
| LEU | CD1 | 327 | −21.2 | −55.4 | 76.0 | 1 | A |
| LEU | CD2 | 327 | −22.3 | −54.6 | 74.0 | 2 | A |
| LEU | C | 327 | −25.1 | −52.6 | 76.8 | 1 | A |
| LEU | O | 327 | −24.9 | −52.1 | 78.0 | 1 | A |
| MET | N | 328 | −26.4 | −52.9 | 76.4 | 1 | A |
| MET | CA | 328 | −27.5 | −52.8 | 77.3 | 2 | A |
| MET | CB | 328 | −28.8 | −53.3 | 76.5 | 3 | A |
| MET | CG | 328 | −30.0 | −53.5 | 77.3 | 1 | A |
| MET | SD | 328 | −31.2 | −54.4 | 76.4 | 1 | A |
| MET | CE | 328 | −32.5 | −53.2 | 76.1 | 3 | A |
| MET | C | 328 | −27.3 | −53.5 | 78.6 | 3 | A |
| MET | O | 328 | −27.1 | −54.7 | 78.6 | 4 | A |
| GLY | N | 329 | −27.5 | −52.7 | 79.7 | 4 | A |
| GLY | CA | 329 | −27.3 | −53.3 | 81.0 | 4 | A |
| GLY | C | 329 | −28.4 | −54.3 | 81.4 | 5 | A |
| GLY | O | 329 | −29.4 | −54.4 | 80.7 | 2 | A |
| GLU | N | 330 | −28.2 | −54.9 | 82.5 | 6 | A |
| GLU | CA | 330 | −29.2 | −55.9 | 83.1 | 7 | A |
| GLU | CB | 330 | −28.5 | −57.0 | 83.8 | 11 | A |
| GLU | CG | 330 | −28.3 | −58.3 | 83.0 | 20 | A |
| GLU | CD | 330 | −29.7 | −58.9 | 82.7 | 26 | A |
| GLU | OE1 | 330 | −29.6 | −60.0 | 82.1 | 30 | A |
| GLU | OE2 | 330 | −30.7 | −58.3 | 83.0 | 31 | A |
| GLU | C | 330 | −30.2 | −55.2 | 83.9 | 5 | A |
| GLU | O | 330 | −31.2 | −55.9 | 84.4 | 4 | A |
| VAL | N | 331 | −30.1 | −53.9 | 84.2 | 6 | A |
| VAL | CA | 331 | −31.1 | −53.2 | 85.0 | 5 | A |
| VAL | CB | 331 | −30.4 | −52.3 | 86.0 | 1 | A |
| VAL | CG1 | 331 | −31.3 | −52.0 | 87.1 | 1 | A |
| VAL | CG2 | 331 | −29.1 | −53.0 | 86.5 | 2 | A |
| VAL | C | 331 | −31.9 | −52.3 | 84.1 | 7 | A |
| VAL | O | 331 | −31.5 | −52.1 | 82.9 | 5 | A |
| THR | N | 332 | −33.0 | −51.8 | 84.5 | 11 | A |
| THR | CA | 332 | −33.8 | −50.9 | 83.7 | 13 | A |
| THR | CB | 332 | −35.2 | −50.8 | 84.2 | 16 | A |
| THR | OG1 | 332 | −35.7 | −52.2 | 84.5 | 21 | A |
| THR | CG2 | 332 | −36.1 | −50.2 | 83.1 | 15 | A |
| THR | C | 332 | −33.2 | −49.5 | 83.6 | 12 | A |
| THR | O | 332 | −32.9 | −48.9 | 84.6 | 14 | A |
| ASN | N | 333 | −33.0 | −49.0 | 82.4 | 8 | A |
| ASN | CA | 333 | −32.4 | −47.7 | 82.2 | 6 | A |
| ASN | CB | 333 | −33.2 | −46.6 | 83.0 | 7 | A |
| ASN | CG | 333 | −34.5 | −46.3 | 82.4 | 9 | A |
| ASN | OD1 | 333 | −34.8 | −46.5 | 81.2 | 12 | A |
| ASN | ND2 | 333 | −35.4 | −45.9 | 83.2 | 10 | A |
| ASN | C | 333 | −30.9 | −47.7 | 82.6 | 3 | A |
| ASN | O | 333 | −30.5 | −46.7 | 83.2 | 7 | A |
| GLN | N | 334 | −30.2 | −48.7 | 82.3 | 7 | A |
| GLN | CA | 334 | −28.8 | −48.8 | 82.7 | 8 | A |
| GLN | CB | 334 | −28.7 | −49.3 | 84.2 | 13 | A |
| GLN | CG | 334 | −27.3 | −49.9 | 84.6 | 17 | A |
| GLN | CD | 334 | −27.1 | −49.8 | 86.1 | 24 | A |
| GLN | OE1 | 334 | −26.3 | −50.6 | 86.6 | 23 | A |
| GLN | NE2 | 334 | −27.9 | −49.0 | 86.8 | 21 | A |
| GLN | C | 334 | −27.9 | −49.7 | 81.8 | 8 | A |
| GLN | O | 334 | −28.4 | −50.7 | 81.4 | 8 | A |
| SER | N | 335 | −26.7 | −49.2 | 81.5 | 7 | A |
| SER | CA | 335 | −25.8 | −49.9 | 80.6 | 1 | A |
| SER | CB | 335 | −25.9 | −49.3 | 79.2 | 1 | A |
| SER | OG | 335 | −25.5 | −48.0 | 79.2 | 1 | A |
| SER | C | 335 | −24.4 | −49.7 | 81.1 | 1 | A |
| SER | O | 335 | −24.2 | −49.0 | 82.1 | 1 | A |
| PHE | N | 336 | −23.5 | −50.4 | 80.5 | 1 | A |
| PHE | CA | 336 | −22.1 | −50.4 | 80.9 | 1 | A |
| PHE | CB | 336 | −21.7 | −51.6 | 81.7 | 1 | A |
| PHE | CG | 336 | −21.8 | −52.9 | 81.0 | 1 | A |
| PHE | CD1 | 336 | −20.7 | −53.3 | 80.2 | 1 | A |
| PHE | CD2 | 336 | −22.9 | −53.7 | 81.1 | 1 | A |
| PHE | CE1 | 336 | −20.8 | −54.5 | 79.5 | 1 | A |
| PHE | CE2 | 336 | −22.9 | −54.9 | 80.4 | 1 | A |
| PHE | CZ | 336 | −21.9 | −55.3 | 79.6 | 1 | A |
| PHE | C | 336 | −21.4 | −50.4 | 79.5 | 1 | A |
| PHE | O | 336 | −22.0 | −50.7 | 78.5 | 1 | A |
| ARG | N | 337 | −20.1 | −50.1 | 79.6 | 1 | A |
| ARG | CA | 337 | −19.3 | −50.0 | 78.3 | 1 | A |
| ARG | CB | 337 | −19.0 | −48.6 | 78.0 | 1 | A |
| ARG | CG | 337 | −17.9 | −48.0 | 78.9 | 3 | A |
| ARG | CD | 337 | −17.7 | −46.6 | 78.7 | 2 | A |
| ARG | NE | 337 | −16.6 | −46.1 | 79.4 | 1 | A |
| ARG | CZ | 337 | −16.4 | −44.8 | 79.9 | 1 | A |
| ARG | NH1 | 337 | −17.4 | −44.0 | 79.7 | 3 | A |
| ARG | NH2 | 337 | −15.4 | −44.5 | 80.6 | 6 | A |
| ARG | C | 337 | −18.1 | −50.8 | 78.5 | 1 | A |
| ARG | O | 337 | −17.5 | −51.0 | 79.6 | 1 | A |
| ILE | N | 338 | −17.6 | −51.3 | 77.3 | 1 | A |
| ILE | CA | 338 | −16.3 | −52.0 | 77.3 | 2 | A |
| ILE | CB | 338 | −16.6 | −53.5 | 76.8 | 1 | A |
| ILE | CG2 | 338 | −17.3 | −54.2 | 77.8 | 1 | A |
| ILE | CG1 | 338 | −17.3 | −53.5 | 75.5 | 1 | A |
| ILE | CD1 | 338 | −17.5 | −54.9 | 74.9 | 1 | A |
| ILE | C | 338 | −15.4 | −51.3 | 76.3 | 3 | A |
| ILE | O | 338 | −15.9 | −50.8 | 75.3 | 4 | A |
| THR | N | 339 | −14.2 | −51.1 | 76.7 | 2 | A |
| THR | CA | 339 | −13.3 | −50.3 | 75.8 | 1 | A |
| THR | CB | 339 | −12.7 | −49.1 | 76.5 | 1 | A |
| THR | OG1 | 339 | −13.8 | −48.4 | 77.1 | 1 | A |
| THR | CG2 | 339 | −12.0 | −48.2 | 75.6 | 1 | A |
| THR | C | 339 | −12.1 | −51.2 | 75.4 | 1 | A |
| THR | O | 339 | −11.5 | −51.9 | 76.1 | 1 | A |
| ILE | N | 340 | −11.8 | −51.2 | 74.1 | 1 | A |
| ILE | CA | 340 | −10.7 | −51.9 | 73.5 | 1 | A |
| ILE | CB | 340 | −11.2 | −52.8 | 72.3 | 1 | A |
| ILE | CG2 | 340 | −12.4 | −53.6 | 72.8 | 1 | A |
| ILE | CG1 | 340 | −11.6 | −51.9 | 71.2 | 1 | A |
| ILE | CD1 | 340 | −12.1 | −52.7 | 70.0 | 1 | A |
| ILE | C | 340 | −9.7 | −50.9 | 72.9 | 1 | A |
| ILE | O | 340 | −10.1 | −49.8 | 72.5 | 1 | A |
| LEU | N | 341 | −8.4 | −51.3 | 72.9 | 1 | A |
| LEU | CA | 341 | −7.4 | −50.4 | 72.5 | 1 | A |
| LEU | CB | 341 | −6.2 | −50.6 | 73.4 | 1 | A |
| LEU | CG | 341 | −6.5 | −50.8 | 74.9 | 3 | A |
| LEU | CD1 | 341 | −5.3 | −51.3 | 75.7 | 1 | A |
| LEU | CD2 | 341 | −7.1 | −49.5 | 75.4 | 2 | A |
| LEU | C | 341 | −7.0 | −50.8 | 71.1 | 1 | A |
| LEU | O | 341 | −7.4 | −51.8 | 70.5 | 1 | A |
| PRO | N | 342 | −6.1 | −49.9 | 70.4 | 1 | A |
| PRO | CD | 342 | −5.3 | −48.8 | 71.0 | 1 | A |
| PRO | CA | 342 | −5.7 | −50.2 | 69.1 | 1 | A |
| PRO | CB | 342 | −4.7 | −49.0 | 68.8 | 1 | A |
| PRO | CG | 342 | −4.1 | −48.8 | 70.1 | 2 | A |
| PRO | C | 342 | −5.0 | −51.5 | 69.0 | 1 | A |
| PRO | O | 342 | −5.1 | −52.3 | 68.0 | 3 | A |
| GLN | N | 343 | −4.3 | −51.9 | 70.1 | 1 | A |
| GLN | CA | 343 | −3.6 | −53.1 | 70.2 | 1 | A |
| GLN | CB | 343 | −3.1 | −53.3 | 71.6 | 1 | A |
| GLN | CG | 343 | −1.8 | −52.6 | 72.0 | 1 | A |
| GLN | CD | 343 | −2.0 | −51.2 | 72.5 | 1 | A |
| GLN | OE1 | 343 | −3.1 | −50.6 | 72.5 | 3 | A |
| GLN | NE2 | 343 | −0.9 | −50.5 | 72.9 | 4 | A |
| GLN | C | 343 | −4.5 | −54.3 | 69.8 | 1 | A |
| GLN | O | 343 | −4.0 | −55.3 | 69.4 | 1 | A |
| GLN | N | 344 | −5.8 | −54.1 | 70.0 | 1 | A |
| GLN | CA | 344 | −6.8 | −55.1 | 69.6 | 3 | A |
| GLN | CB | 344 | −8.0 | −55.0 | 70.6 | 3 | A |
| GLN | CG | 344 | −7.8 | −55.9 | 71.8 | 6 | A |
| GLN | CD | 344 | −7.4 | −55.0 | 73.0 | 7 | A |
| GLN | OE1 | 344 | −7.1 | −55.6 | 74.1 | 9 | A |
| GLN | NE2 | 344 | −7.4 | −53.7 | 72.9 | 7 | A |
| GLN | C | 344 | −7.3 | −55.0 | 68.2 | 2 | A |
| GLN | O | 344 | −7.5 | −56.1 | 67.6 | 2 | A |
| TYR | N | 345 | −7.6 | −53.8 | 67.7 | 2 | A |
| TYR | CA | 345 | −8.1 | −53.7 | 66.3 | 1 | A |
| TYR | CB | 345 | −9.1 | −52.6 | 66.2 | 1 | A |
| TYR | CG | 345 | −8.7 | −51.2 | 66.4 | 1 | A |
| TYR | CD1 | 345 | −8.1 | −50.4 | 65.4 | 1 | A |
| TYR | CE1 | 345 | −7.8 | −49.1 | 65.7 | 1 | A |
| TYR | CD2 | 345 | −8.8 | −50.6 | 67.7 | 1 | A |
| TYR | CE2 | 345 | −8.5 | −49.3 | 67.9 | 1 | A |
| TYR | CZ | 345 | −8.0 | −48.5 | 66.9 | 1 | A |
| TYR | OH | 345 | −7.7 | −47.2 | 67.1 | 1 | A |
| TYR | C | 345 | −7.0 | −53.7 | 65.3 | 3 | A |
| TYR | O | 345 | −7.2 | −53.5 | 64.1 | 5 | A |
| LEU | N | 346 | −5.7 | −53.8 | 65.7 | 1 | A |
| LEU | CA | 346 | −4.6 | −53.8 | 64.8 | 1 | A |
| LEU | CB | 346 | −3.6 | −52.7 | 65.1 | 1 | A |
| LEU | CG | 346 | −4.2 | −51.3 | 64.8 | 1 | A |
| LEU | CD1 | 346 | −3.1 | −50.3 | 64.9 | 1 | A |
| LEU | CD2 | 346 | −4.8 | −51.3 | 63.5 | 1 | A |
| LEU | C | 346 | −4.0 | −55.2 | 65.0 | 1 | A |
| LEU | O | 346 | −3.3 | −55.4 | 66.1 | 5 | A |
| ARG | N | 347 | −4.2 | −56.1 | 64.1 | 1 | A |
| ARG | CA | 347 | −3.7 | −57.5 | 64.3 | 1 | A |
| ARG | CB | 347 | −4.7 | −58.4 | 63.6 | 3 | A |
| ARG | CG | 347 | −4.4 | −59.9 | 63.8 | 8 | A |
| ARG | CD | 347 | −5.6 | −60.7 | 63.5 | 7 | A |
| ARG | NE | 347 | −6.1 | −60.5 | 62.1 | 11 | A |
| ARG | CZ | 347 | −5.4 | −60.9 | 61.0 | 16 | A |
| ARG | NH1 | 347 | −4.2 | −61.5 | 61.1 | 17 | A |
| ARG | NH2 | 347 | −5.9 | −60.6 | 59.8 | 20 | A |
| ARG | C | 347 | −2.3 | −57.6 | 63.7 | 3 | A |
| ARG | O | 347 | −2.1 | −57.3 | 62.5 | 7 | A |
| PRO | N | 348 | −1.4 | −58.1 | 64.5 | 5 | A |
| PRO | CD | 348 | −1.6 | −58.5 | 65.9 | 8 | A |
| PRO | CA | 348 | 0.0 | −58.4 | 64.1 | 5 | A |
| PRO | CB | 348 | 0.6 | −58.9 | 65.4 | 2 | A |
| PRO | CG | 348 | −0.2 | −58.3 | 66.4 | 5 | A |
| PRO | C | 348 | 0.1 | −59.4 | 63.0 | 8 | A |
| PRO | O | 348 | 0.0 | −60.6 | 63.3 | 10 | A |
| VAL | N | 349 | 0.2 | −59.0 | 61.7 | 13 | A |
| VAL | CA | 349 | 0.3 | −59.9 | 60.6 | 18 | A |
| VAL | CB | 349 | −0.3 | −59.4 | 59.3 | 20 | A |
| VAL | CG1 | 349 | −1.7 | −59.8 | 59.2 | 26 | A |
| VAL | CG2 | 349 | −0.2 | −57.9 | 59.3 | 25 | A |
| VAL | C | 349 | 1.8 | −60.2 | 60.4 | 21 | A |
| VAL | O | 349 | 2.5 | −59.3 | 59.9 | 23 | A |
| GLU | N | 350 | 2.2 | −61.3 | 60.8 | 25 | A |
| GLU | CA | 350 | 3.6 | −61.7 | 60.7 | 27 | A |
| GLU | CB | 350 | 3.8 | −63.2 | 60.7 | 23 | A |
| GLU | CG | 350 | 5.0 | −63.7 | 59.9 | 24 | A |
| GLU | CD | 350 | 6.2 | −62.8 | 60.2 | 23 | A |
| GLU | OE1 | 350 | 7.0 | −62.6 | 59.2 | 21 | A |
| GLU | OE2 | 350 | 6.4 | −62.3 | 61.3 | 20 | A |
| GLU | C | 350 | 4.1 | −61.2 | 59.3 | 30 | A |
| GLU | O | 350 | 3.6 | −61.5 | 58.3 | 30 | A |
| ASP | N | 351 | 5.1 | −60.3 | 59.4 | 31 | A |
| ASP | CA | 351 | 5.8 | −59.7 | 58.3 | 33 | A |
| ASP | CB | 351 | 7.2 | −59.3 | 58.6 | 34 | A |
| ASP | CG | 351 | 7.9 | −58.7 | 57.3 | 39 | A |
| ASP | OD1 | 351 | 7.6 | −57.6 | 56.9 | 41 | A |
| ASP | OD2 | 351 | 8.8 | −59.4 | 56.8 | 38 | A |
| ASP | C | 351 | 5.7 | −60.6 | 57.0 | 32 | A |
| ASP | O | 351 | 5.7 | −61.8 | 57.2 | 34 | A |
| VAL | N | 352 | 5.8 | −60.0 | 55.8 | 31 | A |
| VAL | CA | 352 | 5.8 | −60.7 | 54.6 | 33 | A |
| VAL | CB | 352 | 6.2 | −59.9 | 53.4 | 31 | A |
| VAL | CG1 | 352 | 6.2 | −60.7 | 52.1 | 27 | A |
| VAL | CG2 | 352 | 5.4 | −58.6 | 53.2 | 27 | A |
| VAL | C | 352 | 6.6 | −62.0 | 54.7 | 35 | A |
| VAL | O | 352 | 6.1 | −63.1 | 54.7 | 34 | A |
| ALA | N | 353 | 7.9 | −61.8 | 55.0 | 37 | A |
| ALA | CA | 353 | 8.8 | −63.0 | 55.2 | 35 | A |
| ALA | CB | 353 | 9.7 | −63.1 | 54.0 | 35 | A |
| ALA | C | 353 | 9.7 | −62.9 | 56.5 | 36 | A |
| ALA | O | 353 | 10.9 | −63.1 | 56.4 | 36 | A |
| THR | N | 354 | 9.0 | −62.5 | 57.6 | 35 | A |
| THR | CA | 354 | 9.7 | −62.3 | 58.9 | 35 | A |
| THR | CB | 354 | 10.3 | −63.7 | 59.4 | 35 | A |
| THR | OG1 | 354 | 9.5 | −64.8 | 58.9 | 30 | A |
| THR | CG2 | 354 | 10.3 | −63.7 | 61.0 | 27 | A |
| THR | C | 354 | 10.8 | −61.3 | 58.7 | 34 | A |
| THR | O | 354 | 11.9 | −61.6 | 58.3 | 33 | A |
| SER | N | 355 | 10.5 | −60.0 | 59.0 | 32 | A |
| SER | CA | 355 | 11.4 | −58.9 | 59.0 | 30 | A |
| SER | CB | 355 | 11.1 | −57.9 | 57.9 | 24 | A |
| SER | OG | 355 | 10.1 | −57.0 | 58.4 | 16 | A |
| SER | C | 355 | 11.6 | −58.2 | 60.3 | 30 | A |
| SER | O | 355 | 10.9 | −58.6 | 61.3 | 28 | A |
| GLN | N | 356 | 12.5 | −57.3 | 60.4 | 31 | A |
| GLN | CA | 356 | 12.8 | −56.5 | 61.6 | 30 | A |
| GLN | CB | 356 | 14.2 | −55.9 | 61.5 | 34 | A |
| GLN | CG | 356 | 15.3 | −56.9 | 61.3 | 41 | A |
| GLN | CD | 356 | 16.5 | −56.6 | 62.2 | 45 | A |
| GLN | OE1 | 356 | 16.9 | −55.5 | 62.4 | 45 | A |
| GLN | NE2 | 356 | 17.1 | −57.7 | 62.8 | 44 | A |
| GLN | C | 356 | 11.8 | −55.3 | 61.7 | 26 | A |
| GLN | O | 356 | 12.2 | −54.2 | 61.9 | 26 | A |
| ASP | N | 357 | 10.5 | −55.6 | 61.5 | 24 | A |
| ASP | CA | 357 | 9.5 | −54.6 | 61.6 | 20 | A |
| ASP | CB | 357 | 9.1 | −54.0 | 60.2 | 21 | A |
| ASP | CG | 357 | 10.1 | −53.0 | 59.8 | 17 | A |
| ASP | OD1 | 357 | 11.3 | −53.3 | 59.9 | 19 | A |
| ASP | OD2 | 357 | 9.7 | −51.9 | 59.4 | 14 | A |
| ASP | C | 357 | 8.2 | −55.3 | 62.1 | 16 | A |
| ASP | O | 357 | 7.9 | −56.4 | 61.9 | 18 | A |
| ASP | N | 358 | 7.4 | −54.4 | 62.8 | 13 | A |
| ASP | CA | 358 | 6.2 | −54.9 | 63.3 | 12 | A |
| ASP | CB | 358 | 5.9 | −54.2 | 64.7 | 10 | A |
| ASP | CG | 358 | 7.0 | −54.6 | 65.7 | 8 | A |
| ASP | OD1 | 358 | 7.0 | −54.0 | 66.7 | 10 | A |
| ASP | OD2 | 358 | 7.7 | −55.6 | 65.4 | 6 | A |
| ASP | C | 358 | 5.2 | −54.4 | 62.3 | 11 | A |
| ASP | O | 358 | 5.2 | −53.2 | 61.9 | 7 | A |
| CYS | N | 359 | 4.3 | −55.3 | 61.8 | 12 | A |
| CYS | CA | 359 | 3.3 | −55.0 | 60.8 | 12 | A |
| CYS | C | 359 | 1.9 | −55.4 | 61.3 | 9 | A |
| CYS | O | 359 | 1.8 | −56.3 | 62.0 | 10 | A |
| CYS | CB | 359 | 3.6 | −55.7 | 59.5 | 14 | A |
| CYS | SG | 359 | 5.2 | −55.3 | 58.7 | 14 | A |
| TYR | N | 360 | 0.9 | −54.6 | 60.9 | 5 | A |
| TYR | CA | 360 | −0.4 | −54.9 | 61.3 | 3 | A |
| TYR | CB | 360 | −0.8 | −54.0 | 62.5 | 4 | A |
| TYR | CG | 360 | 0.2 | −53.9 | 63.6 | 1 | A |
| TYR | CD1 | 360 | 1.4 | −53.1 | 63.4 | 1 | A |
| TYR | CE1 | 360 | 2.3 | −53.1 | 64.4 | 1 | A |
| TYR | CD2 | 360 | 0.0 | −54.6 | 64.8 | 1 | A |
| TYR | CE2 | 360 | 1.0 | −54.5 | 65.8 | 1 | A |
| TYR | CZ | 360 | 2.1 | −53.7 | 65.6 | 1 | A |
| TYR | OH | 360 | 3.0 | −53.7 | 66.6 | 3 | A |
| TYR | C | 360 | −1.5 | −54.7 | 60.3 | 2 | A |
| TYR | O | 360 | −1.4 | −53.9 | 59.4 | 5 | A |
| LYS | N | 361 | −2.5 | −55.5 | 60.4 | 1 | A |
| LYS | CA | 361 | −3.7 | −55.4 | 59.5 | 3 | A |
| LYS | CB | 361 | −4.1 | −56.8 | 59.0 | 5 | A |
| LYS | CG | 361 | −3.4 | −57.1 | 57.7 | 11 | A |
| LYS | CD | 361 | −4.0 | −58.3 | 56.9 | 21 | A |
| LYS | CE | 361 | −3.2 | −58.6 | 55.6 | 25 | A |
| LYS | NZ | 361 | −3.6 | −59.8 | 54.9 | 26 | A |
| LYS | C | 361 | −4.8 | −54.8 | 60.3 | 2 | A |
| LYS | O | 361 | −5.1 | −55.1 | 61.5 | 2 | A |
| PHE | N | 362 | −5.5 | −53.8 | 59.7 | 1 | A |
| PHE | CA | 362 | −6.6 | −53.2 | 60.4 | 1 | A |
| PHE | CB | 362 | −7.1 | −52.0 | 59.6 | 2 | A |
| PHE | CG | 362 | −8.2 | −51.2 | 60.2 | 1 | A |
| PHE | CD1 | 362 | −7.9 | −50.5 | 61.4 | 1 | A |
| PHE | CD2 | 362 | −9.4 | −51.1 | 59.7 | 1 | A |
| PHE | CE1 | 362 | −8.8 | −49.7 | 62.0 | 1 | A |
| PHE | CE2 | 362 | −10.4 | −50.3 | 60.3 | 1 | A |
| PHE | CZ | 362 | −10.1 | −49.6 | 61.4 | 1 | A |
| PHE | C | 362 | −7.7 | −54.3 | 60.6 | 2 | A |
| PHE | O | 362 | −8.2 | −54.8 | 59.6 | 1 | A |
| ALA | N | 363 | −8.0 | −54.6 | 61.8 | 1 | A |
| ALA | CA | 363 | −8.9 | −55.7 | 62.1 | 1 | A |
| ALA | CB | 363 | −8.5 | −56.5 | 63.3 | 3 | A |
| ALA | C | 363 | −10.4 | −55.3 | 62.2 | 1 | A |
| ALA | O | 363 | −11.2 | −56.1 | 62.6 | 1 | A |
| ILE | N | 364 | −10.7 | −54.1 | 61.8 | 1 | A |
| ILE | CA | 364 | −12.1 | −53.6 | 61.8 | 1 | A |
| ILE | CB | 364 | −12.3 | −52.3 | 62.5 | 1 | A |
| ILE | CG2 | 364 | −13.8 | −51.9 | 62.5 | 1 | A |
| ILE | CG1 | 364 | −11.8 | −52.3 | 63.9 | 1 | A |
| ILE | CD1 | 364 | −11.9 | −51.0 | 64.6 | 1 | A |
| ILE | C | 364 | −12.6 | −53.6 | 60.4 | 2 | A |
| ILE | O | 364 | −12.0 | −52.9 | 59.6 | 1 | A |
| SER | N | 365 | −13.6 | −54.4 | 60.0 | 1 | A |
| SER | CA | 365 | −14.1 | −54.4 | 58.7 | 1 | A |
| SER | CB | 365 | −13.6 | −55.7 | 58.0 | 1 | A |
| SER | OG | 365 | −14.2 | −56.8 | 58.5 | 1 | A |
| SER | C | 365 | −15.6 | −54.3 | 58.6 | 1 | A |
| SER | O | 365 | −16.3 | −54.6 | 59.6 | 1 | A |
| GLN | N | 366 | −16.1 | −53.9 | 57.5 | 1 | A |
| GLN | CA | 366 | −17.5 | −53.8 | 57.2 | 3 | A |
| GLN | CB | 366 | −17.8 | −52.9 | 56.0 | 7 | A |
| GLN | CG | 366 | −16.9 | −53.3 | 54.8 | 20 | A |
| GLN | CD | 366 | −17.1 | −52.3 | 53.7 | 28 | A |
| GLN | OE1 | 366 | −18.2 | −52.1 | 53.2 | 28 | A |
| GLN | NE2 | 366 | −16.0 | −51.6 | 53.3 | 25 | A |
| GLN | C | 366 | −18.3 | −55.1 | 57.1 | 1 | A |
| GLN | O | 366 | −17.7 | −56.1 | 56.5 | 1 | A |
| SER | N | 367 | −19.5 | −55.1 | 57.5 | 2 | A |
| SER | CA | 367 | −20.4 | −56.3 | 57.4 | 1 | A |
| SER | CB | 367 | −20.6 | −56.8 | 58.8 | 5 | A |
| SER | OG | 367 | −21.8 | −57.7 | 58.8 | 3 | A |
| SER | C | 367 | −21.7 | −56.0 | 56.8 | 1 | A |
| SER | O | 367 | −22.2 | −54.9 | 56.9 | 4 | A |
| SER | N | 368 | −22.3 | −57.0 | 56.2 | 4 | A |
| SER | CA | 368 | −23.6 | −56.8 | 55.6 | 5 | A |
| SER | CB | 368 | −23.6 | −57.4 | 54.2 | 2 | A |
| SER | OG | 368 | −23.4 | −58.8 | 54.2 | 8 | A |
| SER | C | 368 | −24.7 | −57.6 | 56.4 | 5 | A |
| SER | O | 368 | −25.8 | −57.5 | 56.1 | 1 | A |
| THR | N | 369 | −24.2 | −58.2 | 57.5 | 6 | A |
| THR | CA | 369 | −25.1 | −59.0 | 58.4 | 6 | A |
| THR | CB | 369 | −24.6 | −60.4 | 58.4 | 4 | A |
| THR | OG1 | 369 | −23.2 | −60.5 | 58.6 | 3 | A |
| THR | CG2 | 369 | −24.9 | −61.1 | 57.0 | 5 | A |
| THR | C | 369 | −25.1 | −58.4 | 59.8 | 5 | A |
| THR | O | 369 | −25.4 | −59.2 | 60.8 | 3 | A |
| GLY | N | 370 | −24.9 | −57.1 | 60.0 | 4 | A |
| GLY | CA | 370 | −25.0 | −56.6 | 61.3 | 8 | A |
| GLY | C | 370 | −23.7 | −56.5 | 62.1 | 9 | A |
| GLY | O | 370 | −22.6 | −56.6 | 61.4 | 10 | A |
| THR | N | 371 | −23.7 | −56.3 | 63.4 | 6 | A |
| THR | CA | 371 | −22.5 | −56.2 | 64.2 | 1 | A |
| THR | CB | 371 | −22.6 | −55.2 | 65.3 | 1 | A |
| THR | OG1 | 371 | −22.6 | −53.9 | 64.9 | 1 | A |
| THR | CG2 | 371 | −21.5 | −55.4 | 66.3 | 1 | A |
| THR | C | 371 | −22.0 | −57.5 | 64.7 | 1 | A |
| THR | O | 371 | −22.8 | −58.3 | 65.2 | 1 | A |
| VAL | N | 372 | −20.8 | −57.8 | 64.5 | 1 | A |
| VAL | CA | 372 | −20.2 | −59.1 | 65.0 | 1 | A |
| VAL | CB | 372 | −19.7 | −59.9 | 63.8 | 1 | A |
| VAL | CG1 | 372 | −19.1 | −61.2 | 64.3 | 1 | A |
| VAL | CG2 | 372 | −20.9 | −60.3 | 62.9 | 1 | A |
| VAL | C | 372 | −19.0 | −58.8 | 65.9 | 1 | A |
| VAL | O | 372 | −18.0 | −58.2 | 65.5 | 1 | A |
| MET | N | 373 | −19.1 | −59.2 | 67.2 | 1 | A |
| MET | CA | 373 | −18.0 | −59.0 | 68.1 | 1 | A |
| MET | CB | 373 | −18.6 | −58.8 | 69.5 | 1 | A |
| MET | CG | 373 | −19.0 | −57.4 | 69.8 | 2 | A |
| MET | SD | 373 | −20.4 | −57.1 | 71.0 | 1 | A |
| MET | CE | 373 | −19.5 | −57.0 | 72.5 | 5 | A |
| MET | C | 373 | −17.1 | −60.1 | 68.0 | 1 | A |
| MET | O | 373 | −17.1 | −61.0 | 68.9 | 3 | A |
| GLY | N | 374 | −16.2 | −60.1 | 67.0 | 1 | A |
| GLY | CA | 374 | −15.2 | −61.1 | 66.8 | 1 | A |
| GLY | C | 374 | −14.1 | −61.2 | 67.8 | 1 | A |
| GLY | O | 374 | −14.2 | −60.6 | 68.9 | 6 | A |
| ALA | N | 375 | −13.1 | −61.9 | 67.5 | 2 | A |
| ALA | CA | 375 | −11.9 | −62.1 | 68.3 | 1 | A |
| ALA | CB | 375 | −11.0 | −63.1 | 67.8 | 1 | A |
| ALA | C | 375 | −11.2 | −60.9 | 68.7 | 1 | A |
| ALA | O | 375 | −10.5 | −60.9 | 69.7 | 1 | A |
| VAL | N | 376 | −11.3 | −59.8 | 68.0 | 1 | A |
| VAL | CA | 376 | −10.5 | −58.6 | 68.4 | 2 | A |
| VAL | CB | 376 | −10.5 | −57.5 | 67.3 | 1 | A |
| VAL | CG1 | 376 | −10.2 | −58.1 | 66.0 | 1 | A |
| VAL | CG2 | 376 | −11.7 | −56.6 | 67.3 | 1 | A |
| VAL | C | 376 | −11.2 | −58.0 | 69.6 | 3 | A |
| VAL | O | 376 | −10.6 | −57.6 | 70.6 | 5 | A |
| ILE | N | 377 | −12.5 | −58.0 | 69.7 | 3 | A |
| ILE | CA | 377 | −13.3 | −57.5 | 70.8 | 3 | A |
| ILE | CB | 377 | −14.8 | −57.3 | 70.5 | 5 | A |
| ILE | CG2 | 377 | −15.6 | −57.1 | 71.8 | 6 | A |
| ILE | CG1 | 377 | −15.0 | −56.2 | 69.5 | 3 | A |
| ILE | CD1 | 377 | −14.5 | −54.9 | 69.9 | 4 | A |
| ILE | C | 377 | −13.1 | −58.5 | 72.0 | 2 | A |
| ILE | O | 377 | −13.0 | −58.1 | 73.1 | 4 | A |
| MET | N | 378 | −13.1 | −59.8 | 71.6 | 1 | A |
| MET | CA | 378 | −12.9 | −60.8 | 72.7 | 1 | A |
| MET | CB | 378 | −13.3 | −62.1 | 72.2 | 1 | A |
| MET | CG | 378 | −14.8 | −62.2 | 71.9 | 1 | A |
| MET | SD | 378 | −15.3 | −63.9 | 71.4 | 1 | A |
| MET | CE | 378 | −16.2 | −64.4 | 72.9 | 2 | A |
| MET | C | 378 | −11.5 | −60.8 | 73.2 | 4 | A |
| MET | O | 378 | −11.3 | −61.0 | 74.4 | 10 | A |
| GLU | N | 379 | −10.5 | −60.7 | 72.3 | 3 | A |
| GLU | CA | 379 | −9.1 | −60.7 | 72.7 | 3 | A |
| GLU | CB | 379 | −8.2 | −60.5 | 71.6 | 5 | A |
| GLU | CG | 379 | −7.9 | −61.7 | 70.7 | 12 | A |
| GLU | CD | 379 | −6.8 | −61.4 | 69.6 | 20 | A |
| GLU | OE1 | 379 | −6.6 | −62.3 | 68.8 | 20 | A |
| GLU | OE2 | 379 | −6.2 | −60.4 | 69.7 | 22 | A |
| GLU | C | 379 | −8.8 | −59.6 | 73.8 | 1 | A |
| GLU | O | 379 | −7.8 | −59.6 | 74.3 | 3 | A |
| GLY | N | 380 | −9.8 | −58.8 | 74.0 | 1 | A |
| GLY | CA | 380 | −9.7 | −57.8 | 75.0 | 1 | A |
| GLY | C | 380 | −10.2 | −58.1 | 76.4 | 1 | A |
| GLY | O | 380 | −9.7 | −57.7 | 77.4 | 1 | A |
| PHE | N | 381 | −11.3 | −58.9 | 76.5 | 1 | A |
| PHE | CA | 381 | −11.9 | −59.2 | 77.8 | 1 | A |
| PHE | CB | 381 | −13.2 | −58.5 | 77.9 | 3 | A |
| PHE | CG | 381 | −13.2 | −57.2 | 77.2 | 1 | A |
| PHE | CD1 | 381 | −13.4 | −57.1 | 75.9 | 1 | A |
| PHE | CD2 | 381 | −12.8 | −56.0 | 77.9 | 2 | A |
| PHE | CE1 | 381 | −13.4 | −55.9 | 75.2 | 1 | A |
| PHE | CE2 | 381 | −12.8 | −54.8 | 77.3 | 1 | A |
| PHE | CZ | 381 | −13.0 | −54.7 | 75.9 | 1 | A |
| PHE | C | 381 | −12.1 | −60.7 | 78.0 | 1 | A |
| PHE | O | 381 | −11.9 | −61.6 | 77.2 | 1 | A |
| TYR | N | 382 | −12.6 | −61.0 | 79.2 | 1 | A |
| TYR | CA | 382 | −12.9 | −62.3 | 79.7 | 1 | A |
| TYR | CB | 382 | −12.6 | −62.5 | 81.1 | 1 | A |
| TYR | CG | 382 | −12.9 | −63.8 | 81.7 | 1 | A |
| TYR | CD1 | 382 | −12.3 | −65.0 | 81.2 | 1 | A |
| TYR | CE1 | 382 | −12.6 | −66.2 | 81.7 | 1 | A |
| TYR | CD2 | 382 | −13.8 | −64.0 | 82.8 | 1 | A |
| TYR | CE2 | 382 | −14.1 | −65.3 | 83.3 | 1 | A |
| TYR | CZ | 382 | −13.4 | −66.4 | 82.8 | 1 | A |
| TYR | OH | 382 | −13.7 | −67.6 | 83.3 | 1 | A |
| TYR | C | 382 | −14.4 | −62.3 | 79.4 | 1 | A |
| TYR | O | 382 | −15.1 | −61.5 | 79.9 | 2 | A |
| VAL | N | 383 | −14.8 | −63.2 | 78.5 | 1 | A |
| VAL | CA | 383 | −16.2 | −63.3 | 78.2 | 1 | A |
| VAL | CB | 383 | −16.4 | −63.3 | 76.7 | 1 | A |
| VAL | CG1 | 383 | −17.9 | −63.3 | 76.3 | 1 | A |
| VAL | CG2 | 383 | −15.7 | −62.2 | 76.0 | 1 | A |
| VAL | C | 383 | −16.9 | −64.5 | 78.8 | 1 | A |
| VAL | O | 383 | −16.4 | −65.6 | 78.8 | 1 | A |
| VAL | N | 384 | −18.0 | −64.2 | 79.5 | 2 | A |
| VAL | CA | 384 | −18.8 | −65.3 | 80.2 | 1 | A |
| VAL | CB | 384 | −19.1 | −64.9 | 81.6 | 1 | A |
| VAL | CG1 | 384 | −20.0 | −65.9 | 82.2 | 1 | A |
| VAL | CG2 | 384 | −17.8 | −65.0 | 82.4 | 1 | A |
| VAL | C | 384 | −20.1 | −65.5 | 79.5 | 1 | A |
| VAL | O | 384 | −20.9 | −64.6 | 79.4 | 1 | A |
| PHE | N | 385 | −20.4 | −66.7 | 79.0 | 1 | A |
| PHE | CA | 385 | −21.6 | −67.1 | 78.4 | 1 | A |
| PHE | CB | 385 | −21.3 | −68.0 | 77.2 | 1 | A |
| PHE | CG | 385 | −20.5 | −67.2 | 76.1 | 1 | A |
| PHE | CD1 | 385 | −21.2 | −66.7 | 75.0 | 2 | A |
| PHE | CD2 | 385 | −19.2 | −67.0 | 76.2 | 1 | A |
| PHE | CE1 | 385 | −20.5 | −66.0 | 74.0 | 4 | A |
| PHE | CE2 | 385 | −18.5 | −66.3 | 75.3 | 3 | A |
| PHE | CZ | 385 | −19.1 | −65.8 | 74.2 | 2 | A |
| PHE | C | 385 | −22.5 | −67.8 | 79.4 | 1 | A |
| PHE | O | 385 | −22.5 | −69.0 | 79.4 | 3 | A |
| ASP | N | 386 | −23.2 | −67.0 | 80.2 | 1 | A |
| ASP | CA | 386 | −24.1 | −67.5 | 81.2 | 1 | A |
| ASP | CB | 386 | −24.4 | −66.4 | 82.2 | 3 | A |
| ASP | CG | 386 | −24.7 | −67.0 | 83.6 | 3 | A |
| ASP | OD1 | 386 | −24.8 | −68.2 | 83.8 | 5 | A |
| ASP | OD2 | 386 | −24.9 | −66.2 | 84.5 | 4 | A |
| ASP | C | 386 | −25.4 | −68.0 | 80.5 | 1 | A |
| ASP | O | 386 | −26.4 | −67.4 | 80.8 | 1 | A |
| ARG | N | 387 | −25.3 | −69.0 | 79.7 | 2 | A |
| ARG | CA | 387 | −26.5 | −69.5 | 79.0 | 3 | A |
| ARG | CB | 387 | −26.2 | −70.8 | 78.4 | 1 | A |
| ARG | CG | 387 | −25.3 | −70.7 | 77.1 | 1 | A |
| ARG | CD | 387 | −24.4 | −71.9 | 76.9 | 1 | A |
| ARG | NE | 387 | −25.2 | −73.1 | 76.7 | 3 | A |
| ARG | CZ | 387 | −25.9 | −73.4 | 75.6 | 3 | A |
| ARG | NH1 | 387 | −25.9 | −72.6 | 74.6 | 1 | A |
| ARG | NH2 | 387 | −26.5 | −74.6 | 75.6 | 1 | A |
| ARG | C | 387 | −27.6 | −69.6 | 80.0 | 3 | A |
| ARG | O | 387 | −28.7 | −69.1 | 79.8 | 5 | A |
| ALA | N | 388 | −27.4 | −70.4 | 81.1 | 4 | A |
| ALA | CA | 388 | −28.4 | −70.6 | 82.1 | 7 | A |
| ALA | CB | 388 | −27.7 | −71.2 | 83.4 | 10 | A |
| ALA | C | 388 | −29.2 | −69.4 | 82.5 | 8 | A |
| ALA | O | 388 | −30.4 | −69.4 | 82.3 | 10 | A |
| ARG | N | 389 | −28.5 | −68.3 | 82.9 | 8 | A |
| ARG | CA | 389 | −29.2 | −67.1 | 83.3 | 8 | A |
| ARG | CB | 389 | −28.4 | −66.4 | 84.4 | 10 | A |
| ARG | CG | 389 | −28.2 | −67.2 | 85.7 | 15 | A |
| ARG | CD | 389 | −27.8 | −66.3 | 86.8 | 16 | A |
| ARG | NE | 389 | −28.8 | −65.3 | 87.2 | 18 | A |
| ARG | CZ | 389 | −28.6 | −64.4 | 88.2 | 22 | A |
| ARG | NH1 | 389 | −27.5 | −64.4 | 88.9 | 23 | A |
| ARG | NH2 | 389 | −29.6 | −63.5 | 88.4 | 21 | A |
| ARG | C | 389 | −29.5 | −66.2 | 82.1 | 8 | A |
| ARG | O | 389 | −30.0 | −65.1 | 82.3 | 9 | A |
| LYS | N | 390 | −29.2 | −66.7 | 80.9 | 9 | A |
| LYS | CA | 390 | −29.6 | −66.0 | 79.7 | 6 | A |
| LYS | CB | 390 | −31.1 | −65.8 | 79.6 | 7 | A |
| LYS | CG | 390 | −31.7 | −65.5 | 78.3 | 19 | A |
| LYS | CD | 390 | −33.2 | −65.4 | 78.4 | 25 | A |
| LYS | CE | 390 | −33.8 | −65.0 | 77.0 | 30 | A |
| LYS | NZ | 390 | −35.3 | −64.9 | 77.1 | 31 | A |
| LYS | C | 390 | −28.9 | −64.6 | 79.6 | 3 | A |
| LYS | O | 390 | −29.6 | −63.6 | 79.6 | 5 | A |
| ARG | N | 391 | −27.6 | −64.5 | 79.7 | 2 | A |
| ARG | CA | 391 | −26.9 | −63.3 | 79.7 | 1 | A |
| ARG | CB | 391 | −27.0 | −62.6 | 81.0 | 2 | A |
| ARG | CG | 391 | −26.5 | −63.4 | 82.2 | 6 | A |
| ARG | CD | 391 | −26.4 | −62.7 | 83.5 | 7 | A |
| ARG | NE | 391 | −25.8 | −63.6 | 84.5 | 1 | A |
| ARG | CZ | 391 | −25.6 | −63.2 | 85.8 | 1 | A |
| ARG | NH1 | 391 | −25.9 | −62.0 | 86.2 | 2 | A |
| ARG | NH2 | 391 | −25.0 | −64.1 | 86.6 | 2 | A |
| ARG | C | 391 | −25.4 | −63.4 | 79.4 | 1 | A |
| ARG | O | 391 | −24.9 | −64.5 | 79.5 | 4 | A |
| ILE | N | 392 | −24.7 | −62.3 | 79.0 | 1 | A |
| ILE | CA | 392 | −23.3 | −62.4 | 78.7 | 1 | A |
| ILE | CB | 392 | −23.0 | −62.2 | 77.3 | 1 | A |
| ILE | CG2 | 392 | −21.6 | −61.9 | 77.0 | 1 | A |
| ILE | CG1 | 392 | −23.6 | −63.3 | 76.5 | 1 | A |
| ILE | CD1 | 392 | −23.4 | −63.2 | 75.0 | 1 | A |
| ILE | C | 392 | −22.6 | −61.4 | 79.6 | 1 | A |
| ILE | O | 392 | −23.0 | −60.3 | 79.7 | 1 | A |
| GLY | N | 393 | −21.4 | −61.8 | 80.1 | 2 | A |
| GLY | CA | 393 | −20.6 | −60.9 | 80.9 | 1 | A |
| GLY | C | 393 | −19.2 | −60.7 | 80.4 | 1 | A |
| GLY | O | 393 | −18.6 | −61.6 | 79.9 | 1 | A |
| PHE | N | 394 | −18.8 | −59.5 | 80.5 | 2 | A |
| PHE | CA | 394 | −17.4 | −59.1 | 80.1 | 3 | A |
| PHE | CB | 394 | −17.5 | −58.0 | 79.0 | 1 | A |
| PHE | CG | 394 | −18.2 | −58.4 | 77.8 | 1 | A |
| PHE | CD1 | 394 | −19.6 | −58.6 | 77.8 | 1 | A |
| PHE | CD2 | 394 | −17.6 | −58.4 | 76.5 | 1 | A |
| PHE | CE1 | 394 | −20.3 | −58.9 | 76.6 | 1 | A |
| PHE | CE2 | 394 | −18.3 | −58.6 | 75.4 | 1 | A |
| PHE | CZ | 394 | −19.6 | −58.8 | 75.4 | 1 | A |
| PHE | C | 394 | −16.7 | −58.4 | 81.3 | 5 | A |
| PHE | O | 394 | −17.2 | −57.6 | 82.0 | 3 | A |
| ALA | N | 395 | −15.4 | −58.8 | 81.4 | 4 | A |
| ALA | CA | 395 | −14.5 | −58.2 | 82.4 | 4 | A |
| ALA | CB | 395 | −14.4 | −59.2 | 83.6 | 8 | A |
| ALA | C | 395 | −13.2 | −58.1 | 81.7 | 1 | A |
| ALA | O | 395 | −12.9 | −58.8 | 80.8 | 1 | A |
| VAL | N | 396 | −12.3 | −57.2 | 82.3 | 2 | A |
| VAL | CA | 396 | −11.0 | −57.0 | 81.7 | 3 | A |
| VAL | CB | 396 | −10.2 | −55.8 | 82.3 | 2 | A |
| VAL | CG1 | 396 | −9.1 | −55.5 | 81.5 | 1 | A |
| VAL | CG2 | 396 | −11.2 | −54.7 | 82.4 | 3 | A |
| VAL | C | 396 | −10.1 | −58.2 | 81.7 | 3 | A |
| VAL | O | 396 | −9.7 | −58.7 | 82.8 | 1 | A |
| SER | N | 397 | −9.8 | −58.7 | 80.5 | 4 | A |
| SER | CA | 397 | −8.9 | −59.9 | 80.3 | 8 | A |
| SER | CB | 397 | −8.8 | −60.2 | 78.8 | 12 | A |
| SER | OG | 397 | −8.0 | −61.4 | 78.6 | 15 | A |
| SER | C | 397 | −7.5 | −59.7 | 80.9 | 9 | A |
| SER | O | 397 | −6.8 | −58.8 | 80.4 | 9 | A |
| ALA | N | 398 | −7.2 | −60.5 | 81.8 | 9 | A |
| ALA | CA | 398 | −5.9 | −60.5 | 82.5 | 9 | A |
| ALA | CB | 398 | −5.8 | −61.4 | 83.7 | 14 | A |
| ALA | C | 398 | −4.7 | −60.8 | 81.5 | 11 | A |
| ALA | O | 398 | −3.6 | −60.8 | 81.9 | 12 | A |
| CYS | N | 399 | −5.1 | −61.2 | 80.3 | 9 | A |
| CYS | CA | 399 | −4.1 | −61.5 | 79.3 | 7 | A |
| CYS | C | 399 | −4.2 | −60.7 | 78.1 | 5 | A |
| CYS | O | 399 | −3.7 | −61.1 | 77.0 | 11 | A |
| CYS | CB | 399 | −4.2 | −63.0 | 78.9 | 8 | A |
| CYS | SG | 399 | −5.7 | −63.3 | 77.9 | 19 | A |
| HIS | N | 400 | −4.7 | −59.5 | 78.2 | 5 | A |
| HIS | CA | 400 | −4.9 | −58.7 | 77.0 | 6 | A |
| HIS | CB | 400 | −6.1 | −57.8 | 77.0 | 7 | A |
| HIS | CG | 400 | −6.0 | −56.5 | 77.7 | 1 | A |
| HIS | CD2 | 400 | −5.8 | −55.2 | 77.2 | 2 | A |
| HIS | ND1 | 400 | −6.0 | −56.3 | 79.1 | 1 | A |
| HIS | CE1 | 400 | −5.9 | −55.0 | 79.4 | 1 | A |
| HIS | NE2 | 400 | −5.7 | −54.4 | 78.2 | 1 | A |
| HIS | C | 400 | −3.7 | −57.8 | 76.8 | 6 | A |
| HIS | O | 400 | −3.0 | −57.3 | 77.7 | 8 | A |
| VAL | N | 401 | −3.4 | −57.5 | 75.5 | 6 | A |
| VAL | CA | 401 | −2.2 | −56.7 | 75.2 | 7 | A |
| VAL | CB | 401 | −1.8 | −56.8 | 73.7 | 6 | A |
| VAL | CG1 | 401 | −0.6 | −56.0 | 73.4 | 6 | A |
| VAL | CG2 | 401 | −1.6 | −58.3 | 73.3 | 10 | A |
| VAL | C | 401 | −2.6 | −55.2 | 75.4 | 6 | A |
| VAL | O | 401 | −3.8 | −54.8 | 75.1 | 5 | A |
| HIS | N | 402 | −1.7 | −54.5 | 76.0 | 7 | A |
| HIS | CA | 402 | −2.0 | −53.0 | 76.3 | 7 | A |
| HIS | CB | 402 | −2.8 | −52.9 | 77.5 | 6 | A |
| HIS | CG | 402 | −2.1 | −53.3 | 78.8 | 4 | A |
| HIS | CD2 | 402 | −1.8 | −52.7 | 79.9 | 5 | A |
| HIS | ND1 | 402 | −1.7 | −54.6 | 79.0 | 5 | A |
| HIS | CE1 | 402 | −1.1 | −54.7 | 80.2 | 8 | A |
| HIS | NE2 | 402 | −1.2 | −53.5 | 80.7 | 4 | A |
| HIS | C | 402 | −0.6 | −52.4 | 76.6 | 7 | A |
| HIS | O | 402 | 0.3 | −53.0 | 77.1 | 10 | A |
| ASP | N | 403 | −0.5 | −51.1 | 76.2 | 6 | A |
| ASP | CA | 403 | 0.7 | −50.5 | 76.4 | 7 | A |
| ASP | CB | 403 | 0.9 | −49.3 | 75.5 | 9 | A |
| ASP | CG | 403 | −0.2 | −48.3 | 75.7 | 9 | A |
| ASP | OD1 | 403 | −0.4 | −47.8 | 76.8 | 13 | A |
| ASP | OD2 | 403 | −0.9 | −47.9 | 74.7 | 8 | A |
| ASP | C | 403 | 0.8 | −50.0 | 77.9 | 9 | A |
| ASP | O | 403 | −0.1 | −50.2 | 78.7 | 9 | A |
| GLU | N | 404 | 1.9 | −49.3 | 78.2 | 12 | A |
| GLU | CA | 404 | 2.2 | −48.8 | 79.5 | 9 | A |
| GLU | CB | 404 | 3.5 | −48.1 | 79.5 | 7 | A |
| GLU | CG | 404 | 3.7 | −47.0 | 80.6 | 7 | A |
| GLU | CD | 404 | 5.0 | −46.3 | 80.6 | 9 | A |
| GLU | OE1 | 404 | 5.5 | −45.9 | 79.5 | 12 | A |
| GLU | OE2 | 404 | 5.7 | −46.1 | 81.6 | 12 | A |
| GLU | C | 404 | 1.1 | −47.8 | 80.0 | 8 | A |
| GLU | O | 404 | 0.8 | −47.8 | 81.2 | 8 | A |
| PHE | N | 405 | 0.6 | −47.0 | 79.1 | 6 | A |
| PHE | CA | 405 | −0.4 | −45.9 | 79.4 | 4 | A |
| PHE | CB | 405 | −0.2 | −44.7 | 78.5 | 3 | A |
| PHE | CG | 405 | 1.2 | −44.2 | 78.4 | 2 | A |
| PHE | CD1 | 405 | 2.1 | −44.8 | 77.5 | 1 | A |
| PHE | CD2 | 405 | 1.6 | −43.3 | 79.3 | 5 | A |
| PHE | CE1 | 405 | 3.4 | −44.4 | 77.5 | 2 | A |
| PHE | CE2 | 405 | 3.0 | −42.8 | 79.3 | 2 | A |
| PHE | CZ | 405 | 3.8 | −43.4 | 78.4 | 4 | A |
| PHE | C | 405 | −1.8 | −46.3 | 79.5 | 5 | A |
| PHE | O | 405 | −2.5 | −46.1 | 80.5 | 9 | A |
| ARG | N | 406 | −2.4 | −46.9 | 78.4 | 4 | A |
| ARG | CA | 406 | −3.8 | −47.2 | 78.4 | 2 | A |
| ARG | CB | 406 | −4.4 | −46.8 | 77.1 | 4 | A |
| ARG | CG | 406 | −4.4 | −45.4 | 76.8 | 8 | A |
| ARG | CD | 406 | −5.1 | −45.0 | 75.5 | 6 | A |
| ARG | NE | 406 | −4.6 | −45.7 | 74.4 | 1 | A |
| ARG | CZ | 406 | −4.7 | −45.2 | 73.2 | 2 | A |
| ARG | NH1 | 406 | −5.3 | −44.1 | 73.0 | 3 | A |
| ARG | NH2 | 406 | −4.2 | −45.9 | 72.1 | 2 | A |
| ARG | C | 406 | −4.0 | −48.7 | 78.6 | 2 | A |
| ARG | O | 406 | −3.1 | −49.5 | 78.7 | 5 | A |
| THR | N | 407 | −5.3 | −49.1 | 78.8 | 1 | A |
| THR | CA | 407 | −5.6 | −50.4 | 79.0 | 2 | A |
| THR | CB | 407 | −5.5 | −50.8 | 80.5 | 4 | A |
| THR | OG1 | 407 | −5.8 | −52.1 | 80.7 | 4 | A |
| THR | CG2 | 407 | −6.3 | −49.9 | 81.4 | 6 | A |
| THR | C | 407 | −7.1 | −50.7 | 78.6 | 1 | A |
| THR | O | 407 | −7.9 | −49.8 | 78.8 | 2 | A |
| ALA | N | 408 | −7.4 | −51.9 | 78.2 | 1 | A |
| ALA | CA | 408 | −8.7 | −52.3 | 77.8 | 1 | A |
| ALA | CB | 408 | −8.8 | −53.7 | 77.5 | 1 | A |
| ALA | C | 408 | −9.5 | −52.0 | 79.1 | 1 | A |
| ALA | O | 408 | −8.9 | −51.9 | 80.2 | 6 | A |
| ALA | N | 409 | −10.8 | −52.0 | 79.1 | 1 | A |
| ALA | CA | 409 | −11.5 | −51.7 | 80.3 | 2 | A |
| ALA | CB | 409 | −11.3 | −50.3 | 80.8 | 4 | A |
| ALA | C | 409 | −13.0 | −52.0 | 80.2 | 3 | A |
| ALA | O | 409 | −13.6 | −51.9 | 79.1 | 5 | A |
| VAL | N | 410 | −13.7 | −52.2 | 81.3 | 1 | A |
| VAL | CA | 410 | −15.1 | −52.5 | 81.4 | 1 | A |
| VAL | CB | 410 | −15.4 | −53.9 | 81.8 | 1 | A |
| VAL | CG1 | 410 | −16.8 | −54.2 | 81.8 | 1 | A |
| VAL | CG2 | 410 | −14.7 | −54.8 | 80.9 | 1 | A |
| VAL | C | 410 | −15.6 | −51.6 | 82.5 | 1 | A |
| VAL | O | 410 | −15.2 | −51.7 | 83.7 | 1 | A |
| GLU | N | 411 | −16.6 | −50.7 | 82.2 | 1 | A |
| GLU | CA | 411 | −17.1 | −49.7 | 83.1 | 3 | A |
| GLU | CB | 411 | −16.3 | −48.4 | 83.0 | 5 | A |
| GLU | CG | 411 | −14.9 | −48.5 | 83.3 | 5 | A |
| GLU | CD | 411 | −14.1 | −47.3 | 82.9 | 6 | A |
| GLU | OE1 | 411 | −13.2 | −46.9 | 83.6 | 13 | A |
| GLU | OE2 | 411 | −14.5 | −46.7 | 81.8 | 7 | A |
| GLU | C | 411 | −18.6 | −49.5 | 83.1 | 4 | A |
| GLU | O | 411 | −19.2 | −49.4 | 82.0 | 2 | A |
| GLY | N | 412 | −19.2 | −49.2 | 84.3 | 5 | A |
| GLY | CA | 412 | −20.6 | −48.9 | 84.4 | 5 | A |
| GLY | C | 412 | −20.9 | −48.2 | 85.7 | 4 | A |
| GLY | O | 412 | −20.0 | −48.0 | 86.5 | 5 | A |
| PRO | N | 413 | −22.1 | −47.8 | 85.9 | 1 | A |
| PRO | CD | 413 | −22.6 | −47.4 | 87.3 | 1 | A |
| PRO | CA | 413 | −23.3 | −47.9 | 85.0 | 2 | A |
| PRO | CB | 413 | −24.4 | −48.2 | 85.9 | 1 | A |
| PRO | CG | 413 | −24.1 | −47.3 | 87.1 | 1 | A |
| PRO | C | 413 | −23.5 | −46.6 | 84.3 | 4 | A |
| PRO | O | 413 | −23.0 | −45.6 | 84.7 | 9 | A |
| PHE | N | 414 | −24.2 | −46.7 | 83.2 | 6 | A |
| PHE | CA | 414 | −24.5 | −45.5 | 82.4 | 8 | A |
| PHE | CB | 414 | −23.7 | −45.5 | 81.1 | 10 | A |
| PHE | CG | 414 | −22.2 | −45.5 | 81.2 | 9 | A |
| PHE | CD1 | 414 | −21.6 | −46.8 | 81.2 | 13 | A |
| PHE | CD2 | 414 | −21.5 | −44.4 | 81.5 | 10 | A |
| PHE | CE1 | 414 | −20.2 | −46.9 | 81.4 | 12 | A |
| PHE | CE2 | 414 | −20.1 | −44.5 | 81.8 | 11 | A |
| PHE | CZ | 414 | −19.5 | −45.7 | 81.7 | 12 | A |
| PHE | C | 414 | −26.0 | −45.3 | 82.1 | 10 | A |
| PHE | O | 414 | −26.6 | −46.2 | 81.6 | 11 | A |
| VAL | N | 415 | −26.5 | −44.2 | 82.5 | 11 | A |
| VAL | CA | 415 | −28.0 | −44.0 | 82.3 | 10 | A |
| VAL | CB | 415 | −28.4 | −42.6 | 82.8 | 10 | A |
| VAL | CG1 | 415 | −30.0 | −42.7 | 83.0 | 8 | A |
| VAL | CG2 | 415 | −27.8 | −42.4 | 84.2 | 16 | A |
| VAL | C | 415 | −28.2 | −43.9 | 80.8 | 10 | A |
| VAL | O | 415 | −27.6 | −43.2 | 80.0 | 9 | A |
| THR | N | 416 | −29.1 | −44.8 | 80.3 | 11 | A |
| THR | CA | 416 | −29.5 | −44.9 | 78.9 | 12 | A |
| THR | CB | 416 | −28.7 | −45.9 | 78.2 | 8 | A |
| THR | OG1 | 416 | −27.3 | −45.7 | 78.5 | 8 | A |
| THR | CG2 | 416 | −28.8 | −45.7 | 76.7 | 8 | A |
| THR | C | 416 | −31.0 | −45.3 | 78.9 | 14 | A |
| THR | O | 416 | −31.3 | −46.3 | 79.5 | 16 | A |
| LEU | N | 417 | −31.8 | −44.5 | 78.3 | 12 | A |
| LEU | CA | 417 | −33.2 | −44.7 | 78.2 | 12 | A |
| LEU | CB | 417 | −33.9 | −43.4 | 78.4 | 10 | A |
| LEU | CG | 417 | −33.5 | −42.5 | 79.5 | 8 | A |
| LEU | CD1 | 417 | −33.8 | −41.1 | 79.2 | 5 | A |
| LEU | CD2 | 417 | −34.2 | −43.0 | 80.8 | 6 | A |
| LEU | C | 417 | −33.6 | −45.4 | 77.0 | 12 | A |
| LEU | O | 417 | −33.0 | −45.2 | 75.9 | 11 | A |
| ASP | N | 418 | −34.7 | −46.2 | 77.0 | 15 | A |
| ASP | CA | 418 | −35.2 | −46.9 | 75.8 | 20 | A |
| ASP | CB | 418 | −35.8 | −45.9 | 74.9 | 26 | A |
| ASP | CG | 418 | −36.9 | −45.1 | 75.5 | 33 | A |
| ASP | OD1 | 418 | −37.1 | −43.9 | 75.0 | 35 | A |
| ASP | OD2 | 418 | −37.6 | −45.6 | 76.5 | 40 | A |
| ASP | C | 418 | −34.1 | −47.7 | 75.1 | 20 | A |
| ASP | O | 418 | −33.9 | −47.5 | 73.9 | 21 | A |
| MET | N | 419 | −33.4 | −48.6 | 75.8 | 17 | A |
| MET | CA | 419 | −32.3 | −49.3 | 75.1 | 13 | A |
| MET | CB | 419 | −31.5 | −50.0 | 76.2 | 10 | A |
| MET | CG | 419 | −30.8 | −49.0 | 77.1 | 10 | A |
| MET | SD | 419 | −29.7 | −49.7 | 78.3 | 13 | A |
| MET | CE | 419 | −30.9 | −50.0 | 79.6 | 13 | A |
| MET | C | 419 | −32.8 | −50.3 | 74.1 | 13 | A |
| MET | O | 419 | −32.1 | −50.6 | 73.1 | 15 | A |
| GLU | N | 420 | −34.0 | −50.9 | 74.3 | 15 | A |
| GLU | CA | 420 | −34.5 | −51.9 | 73.4 | 21 | A |
| GLU | CB | 420 | −35.2 | −53.0 | 74.2 | 25 | A |
| GLU | CG | 420 | −35.2 | −54.4 | 73.5 | 30 | A |
| GLU | CD | 420 | −35.0 | −55.5 | 74.5 | 31 | A |
| GLU | OE1 | 420 | −35.6 | −55.5 | 75.6 | 27 | A |
| GLU | OE2 | 420 | −34.3 | −56.5 | 74.1 | 31 | A |
| GLU | C | 420 | −35.4 | −51.3 | 72.3 | 22 | A |
| GLU | O | 420 | −35.3 | −50.1 | 72.0 | 22 | A |
| GLU | OXT | 420 | −36.2 | −52.0 | 71.8 | 27 | A |
| GLU | CB | 41 | 52.5 | 26.9 | 44.1 | 21 | B |
| GLU | CG | 41 | 51.9 | 25.7 | 44.9 | 26 | B |
| GLU | CD | 41 | 50.8 | 26.1 | 45.8 | 30 | B |
| GLU | OE1 | 41 | 49.9 | 27.0 | 45.4 | 32 | B |
| GLU | OE2 | 41 | 50.7 | 25.6 | 47.0 | 33 | B |
| GLU | C | 41 | 52.8 | 25.4 | 42.2 | 12 | B |
| GLU | O | 41 | 53.2 | 24.3 | 42.4 | 9 | B |
| GLU | N | 41 | 53.7 | 27.8 | 42.1 | 8 | B |
| GLU | CA | 41 | 53.4 | 26.6 | 43.0 | 13 | B |
| MET | N | 42 | 51.8 | 25.7 | 41.3 | 10 | B |
| MET | CA | 42 | 51.2 | 24.7 | 40.4 | 7 | B |
| MET | CB | 42 | 49.7 | 25.0 | 40.3 | 3 | B |
| MET | CG | 42 | 48.9 | 24.5 | 41.6 | 11 | B |
| MET | SD | 42 | 47.1 | 24.9 | 41.6 | 15 | B |
| MET | CE | 42 | 47.1 | 26.3 | 42.6 | 8 | B |
| MET | C | 42 | 51.8 | 24.9 | 39.0 | 6 | B |
| MET | O | 42 | 51.8 | 23.9 | 38.2 | 10 | B |
| VAL | N | 43 | 52.3 | 26.1 | 38.7 | 4 | B |
| VAL | CA | 43 | 52.9 | 26.4 | 37.5 | 2 | B |
| VAL | CB | 43 | 53.8 | 27.6 | 37.6 | 1 | B |
| VAL | CG1 | 43 | 54.5 | 27.9 | 36.3 | 1 | B |
| VAL | CG2 | 43 | 52.9 | 28.8 | 38.0 | 1 | B |
| VAL | C | 43 | 53.8 | 25.2 | 37.0 | 2 | B |
| VAL | O | 43 | 54.4 | 24.5 | 37.8 | 3 | B |
| ASP | N | 44 | 53.8 | 25.0 | 35.7 | 3 | B |
| ASP | CA | 44 | 54.6 | 23.9 | 35.1 | 6 | B |
| ASP | CB | 44 | 56.1 | 24.0 | 35.6 | 10 | B |
| ASP | CG | 44 | 56.9 | 24.7 | 34.5 | 21 | B |
| ASP | OD1 | 44 | 56.9 | 26.0 | 34.4 | 29 | B |
| ASP | OD2 | 44 | 57.6 | 24.0 | 33.8 | 26 | B |
| ASP | C | 44 | 54.2 | 22.5 | 35.6 | 7 | B |
| ASP | O | 44 | 54.9 | 21.5 | 35.4 | 10 | B |
| ASN | N | 45 | 53.0 | 22.4 | 36.1 | 5 | B |
| ASN | CA | 45 | 52.6 | 21.0 | 36.6 | 4 | B |
| ASN | CB | 45 | 51.6 | 21.1 | 37.7 | 4 | B |
| ASN | CG | 45 | 50.3 | 21.7 | 37.3 | 1 | B |
| ASN | OD1 | 45 | 50.2 | 22.3 | 36.2 | 1 | B |
| ASN | ND2 | 45 | 49.3 | 21.5 | 38.1 | 1 | B |
| ASN | C | 45 | 52.1 | 20.1 | 35.5 | 4 | B |
| ASN | O | 45 | 51.9 | 18.9 | 35.7 | 2 | B |
| LEU | N | 46 | 52.0 | 20.6 | 34.2 | 4 | B |
| LEU | CA | 46 | 51.6 | 19.8 | 33.1 | 4 | B |
| LEU | CB | 46 | 50.6 | 20.5 | 32.2 | 1 | B |
| LEU | CG | 46 | 49.4 | 21.0 | 32.9 | 2 | B |
| LEU | CD1 | 46 | 48.4 | 21.4 | 31.9 | 7 | B |
| LEU | CD2 | 46 | 48.8 | 20.0 | 33.8 | 8 | B |
| LEU | C | 46 | 52.8 | 19.4 | 32.2 | 5 | B |
| LEU | O | 46 | 53.8 | 20.2 | 32.2 | 1 | B |
| ARG | N | 47 | 52.7 | 18.3 | 31.6 | 4 | B |
| ARG | CA | 47 | 53.8 | 17.8 | 30.7 | 8 | B |
| ARG | CB | 47 | 54.7 | 16.8 | 31.4 | 14 | B |
| ARG | CG | 47 | 55.8 | 17.4 | 32.2 | 17 | B |
| ARG | CD | 47 | 56.8 | 16.3 | 32.6 | 25 | B |
| ARG | NE | 47 | 56.2 | 15.1 | 33.1 | 35 | B |
| ARG | CZ | 47 | 56.8 | 14.0 | 33.6 | 39 | B |
| ARG | NH1 | 47 | 58.1 | 14.0 | 33.6 | 37 | B |
| ARG | NH2 | 47 | 56.1 | 13.0 | 34.0 | 40 | B |
| ARG | C | 47 | 53.1 | 17.1 | 29.5 | 7 | B |
| ARG | O | 47 | 51.9 | 16.7 | 29.7 | 8 | B |
| GLY | N | 48 | 53.8 | 16.9 | 28.4 | 11 | B |
| GLY | CA | 48 | 53.2 | 16.2 | 27.3 | 12 | B |
| GLY | C | 48 | 53.8 | 16.4 | 26.0 | 15 | B |
| GLY | O | 48 | 54.1 | 17.6 | 25.6 | 19 | B |
| LYS | N | 49 | 54.2 | 15.3 | 25.3 | 16 | B |
| LYS | CA | 49 | 54.8 | 15.5 | 24.0 | 20 | B |
| LYS | CB | 49 | 55.6 | 14.2 | 23.6 | 21 | B |
| LYS | CG | 49 | 54.9 | 12.9 | 24.0 | 24 | B |
| LYS | CD | 49 | 55.8 | 11.7 | 23.9 | 28 | B |
| LYS | CE | 49 | 55.6 | 11.0 | 22.5 | 32 | B |
| LYS | NZ | 49 | 54.4 | 10.2 | 22.3 | 31 | B |
| LYS | C | 49 | 53.9 | 15.8 | 22.8 | 22 | B |
| LYS | O | 49 | 52.9 | 15.1 | 22.5 | 21 | B |
| SER | N | 50 | 54.1 | 16.9 | 22.2 | 24 | B |
| SER | CA | 50 | 53.4 | 17.4 | 21.0 | 26 | B |
| SER | CB | 50 | 54.1 | 17.1 | 19.8 | 28 | B |
| SER | OG | 50 | 53.5 | 17.7 | 18.6 | 34 | B |
| SER | C | 50 | 51.9 | 17.0 | 20.9 | 28 | B |
| SER | O | 50 | 51.0 | 17.5 | 21.6 | 33 | B |
| GLY | N | 51 | 51.6 | 16.2 | 19.9 | 24 | B |
| GLY | CA | 51 | 50.3 | 15.8 | 19.6 | 19 | B |
| GLY | C | 51 | 49.8 | 14.7 | 20.5 | 18 | B |
| GLY | O | 51 | 48.9 | 13.9 | 20.1 | 21 | B |
| GLN | N | 52 | 50.4 | 14.5 | 21.6 | 16 | B |
| GLN | CA | 52 | 50.1 | 13.4 | 22.5 | 14 | B |
| GLN | CB | 52 | 51.3 | 12.8 | 23.1 | 15 | B |
| GLN | CG | 52 | 51.6 | 11.4 | 22.5 | 17 | B |
| GLN | CD | 52 | 52.2 | 11.4 | 21.1 | 15 | B |
| GLN | OE1 | 52 | 52.5 | 10.3 | 20.5 | 17 | B |
| GLN | NE2 | 52 | 52.3 | 12.6 | 20.5 | 20 | B |
| GLN | C | 52 | 49.2 | 13.9 | 23.6 | 13 | B |
| GLN | O | 52 | 48.5 | 13.1 | 24.3 | 18 | B |
| GLY | N | 53 | 49.2 | 15.2 | 23.9 | 13 | B |
| GLY | CA | 53 | 48.3 | 15.7 | 24.9 | 11 | B |
| GLY | C | 53 | 49.0 | 16.1 | 26.2 | 9 | B |
| GLY | O | 53 | 50.2 | 15.7 | 26.5 | 10 | B |
| TYR | N | 54 | 48.3 | 16.9 | 27.1 | 8 | B |
| TYR | CA | 54 | 48.9 | 17.3 | 28.3 | 8 | B |
| TYR | CB | 54 | 48.6 | 18.8 | 28.6 | 7 | B |
| TYR | CG | 54 | 49.0 | 19.7 | 27.5 | 4 | B |
| TYR | CD1 | 54 | 48.2 | 19.8 | 26.4 | 6 | B |
| TYR | CE1 | 54 | 48.7 | 20.6 | 25.3 | 6 | B |
| TYR | CD2 | 54 | 50.2 | 20.3 | 27.6 | 5 | B |
| TYR | CE2 | 54 | 50.7 | 21.1 | 26.5 | 5 | B |
| TYR | CZ | 54 | 49.9 | 21.3 | 25.4 | 3 | B |
| TYR | OH | 54 | 50.3 | 22.1 | 24.4 | 3 | B |
| TYR | C | 54 | 48.4 | 16.5 | 29.5 | 8 | B |
| TYR | O | 54 | 47.2 | 16.0 | 29.5 | 13 | B |
| TYR | N | 55 | 49.3 | 16.3 | 30.5 | 6 | B |
| TYR | CA | 55 | 48.9 | 15.5 | 31.7 | 6 | B |
| TYR | CB | 55 | 49.4 | 14.1 | 31.5 | 9 | B |
| TYR | CG | 55 | 50.9 | 13.9 | 31.4 | 8 | B |
| TYR | CD1 | 55 | 51.8 | 14.0 | 32.4 | 11 | B |
| TYR | CE1 | 55 | 53.2 | 13.8 | 32.3 | 8 | B |
| TYR | CD2 | 55 | 51.5 | 13.7 | 30.1 | 7 | B |
| TYR | CE2 | 55 | 52.8 | 13.5 | 29.9 | 6 | B |
| TYR | CZ | 55 | 53.7 | 13.6 | 31.0 | 6 | B |
| TYR | OH | 55 | 55.0 | 13.5 | 30.8 | 10 | B |
| TYR | C | 55 | 49.5 | 16.1 | 32.9 | 5 | B |
| TYR | O | 55 | 50.5 | 16.8 | 32.9 | 4 | B |
| VAL | N | 56 | 48.8 | 16.0 | 34.0 | 3 | B |
| VAL | CA | 56 | 49.2 | 16.5 | 35.3 | 5 | B |
| VAL | CB | 56 | 48.1 | 17.4 | 35.9 | 1 | B |
| VAL | CG1 | 56 | 46.8 | 16.7 | 36.1 | 1 | B |
| VAL | CG2 | 56 | 48.6 | 18.0 | 37.2 | 2 | B |
| VAL | C | 56 | 49.3 | 15.3 | 36.3 | 9 | B |
| VAL | O | 56 | 48.6 | 14.3 | 36.2 | 11 | B |
| GLU | N | 57 | 50.3 | 15.3 | 37.1 | 12 | B |
| GLU | CA | 57 | 50.5 | 14.2 | 38.0 | 14 | B |
| GLU | CB | 57 | 51.9 | 14.3 | 38.7 | 16 | B |
| GLU | CG | 57 | 52.3 | 13.3 | 39.7 | 26 | B |
| GLU | CD | 57 | 53.8 | 13.2 | 40.0 | 29 | B |
| GLU | OE1 | 57 | 54.5 | 12.7 | 39.2 | 33 | B |
| GLU | OE2 | 57 | 54.1 | 13.6 | 41.1 | 32 | B |
| GLU | C | 57 | 49.5 | 14.1 | 39.1 | 13 | B |
| GLU | O | 57 | 49.1 | 15.1 | 39.7 | 14 | B |
| MET | N | 58 | 49.0 | 12.9 | 39.3 | 11 | B |
| MET | CA | 58 | 47.9 | 12.6 | 40.3 | 9 | B |
| MET | CB | 58 | 46.6 | 12.5 | 39.7 | 9 | B |
| MET | CG | 58 | 46.0 | 13.8 | 39.0 | 1 | B |
| MET | SD | 58 | 44.4 | 13.4 | 38.2 | 1 | B |
| MET | CE | 58 | 43.2 | 13.9 | 39.4 | 1 | B |
| MET | C | 58 | 48.2 | 11.3 | 41.1 | 8 | B |
| MET | O | 58 | 49.0 | 10.5 | 40.7 | 6 | B |
| THR | N | 59 | 47.5 | 11.2 | 42.3 | 10 | B |
| THR | CA | 59 | 47.7 | 10.0 | 43.1 | 11 | B |
| THR | CB | 59 | 48.5 | 10.2 | 44.3 | 14 | B |
| THR | OG1 | 59 | 47.8 | 11.1 | 45.2 | 18 | B |
| THR | CG2 | 59 | 49.8 | 10.9 | 44.0 | 17 | B |
| THR | C | 59 | 46.3 | 9.4 | 43.5 | 10 | B |
| THR | O | 59 | 45.5 | 10.2 | 44.0 | 11 | B |
| VAL | N | 60 | 46.1 | 8.1 | 43.3 | 12 | B |
| VAL | CA | 60 | 44.8 | 7.5 | 43.7 | 10 | B |
| VAL | CB | 60 | 44.1 | 6.9 | 42.5 | 9 | B |
| VAL | CG1 | 60 | 43.9 | 8.0 | 41.4 | 7 | B |
| VAL | CG2 | 60 | 44.9 | 5.8 | 41.9 | 7 | B |
| VAL | C | 60 | 45.1 | 6.4 | 44.7 | 9 | B |
| VAL | O | 60 | 46.2 | 5.8 | 44.8 | 7 | B |
| GLY | N | 61 | 44.0 | 6.0 | 45.4 | 8 | B |
| GLY | CA | 61 | 44.1 | 4.9 | 46.4 | 11 | B |
| GLY | C | 61 | 45.1 | 5.1 | 47.5 | 13 | B |
| GLY | O | 61 | 45.9 | 6.0 | 47.5 | 16 | B |
| SER | N | 62 | 44.9 | 4.2 | 48.6 | 11 | B |
| SER | CA | 62 | 45.8 | 4.3 | 49.7 | 12 | B |
| SER | CB | 62 | 45.0 | 4.6 | 51.0 | 12 | B |
| SER | OG | 62 | 44.3 | 5.9 | 50.9 | 13 | B |
| SER | C | 62 | 46.5 | 3.0 | 50.0 | 13 | B |
| SER | O | 62 | 45.9 | 1.9 | 50.1 | 15 | B |
| PRO | N | 63 | 47.9 | 3.0 | 50.1 | 13 | B |
| PRO | CD | 63 | 48.7 | 1.8 | 50.2 | 12 | B |
| PRO | CA | 63 | 48.8 | 4.2 | 50.0 | 14 | B |
| PRO | CB | 63 | 50.1 | 3.6 | 50.3 | 13 | B |
| PRO | CG | 63 | 50.0 | 2.2 | 49.7 | 14 | B |
| PRO | C | 63 | 48.7 | 4.8 | 48.5 | 15 | B |
| PRO | O | 63 | 48.3 | 4.1 | 47.6 | 19 | B |
| PRO | N | 64 | 49.2 | 6.1 | 48.4 | 12 | B |
| PRO | CD | 64 | 49.7 | 6.9 | 49.5 | 12 | B |
| PRO | CA | 64 | 49.2 | 6.8 | 47.1 | 9 | B |
| PRO | CB | 64 | 49.8 | 8.1 | 47.5 | 9 | B |
| PRO | CG | 64 | 49.4 | 8.3 | 48.9 | 14 | B |
| PRO | C | 64 | 49.9 | 6.2 | 45.9 | 8 | B |
| PRO | O | 64 | 51.1 | 5.9 | 46.0 | 6 | B |
| GLN | N | 65 | 49.2 | 6.0 | 44.8 | 8 | B |
| GLN | CA | 65 | 49.7 | 5.5 | 43.6 | 7 | B |
| GLN | CB | 65 | 48.8 | 4.4 | 42.9 | 6 | B |
| GLN | CG | 65 | 48.7 | 3.1 | 43.7 | 9 | B |
| GLN | CD | 65 | 48.1 | 2.0 | 42.8 | 11 | B |
| GLN | OE1 | 65 | 48.6 | 1.8 | 41.7 | 13 | B |
| GLN | NE2 | 65 | 47.1 | 1.4 | 43.3 | 10 | B |
| GLN | C | 65 | 49.9 | 6.6 | 42.6 | 10 | B |
| GLN | O | 65 | 48.9 | 7.2 | 42.1 | 10 | B |
| THR | N | 66 | 51.1 | 7.0 | 42.2 | 9 | B |
| THR | CA | 66 | 51.4 | 8.1 | 41.3 | 7 | B |
| THR | CB | 66 | 52.8 | 8.4 | 41.4 | 6 | B |
| THR | OG1 | 66 | 53.2 | 8.8 | 42.7 | 8 | B |
| THR | CG2 | 66 | 53.1 | 9.6 | 40.4 | 12 | B |
| THR | C | 66 | 51.0 | 7.7 | 39.9 | 7 | B |
| THR | O | 66 | 51.3 | 6.6 | 39.5 | 10 | B |
| LEU | N | 67 | 50.3 | 8.5 | 39.2 | 7 | B |
| LEU | CA | 67 | 49.8 | 8.3 | 37.8 | 5 | B |
| LEU | CB | 67 | 48.5 | 7.6 | 37.8 | 1 | B |
| LEU | CG | 67 | 48.5 | 6.1 | 38.1 | 3 | B |
| LEU | CD1 | 67 | 47.0 | 5.7 | 38.2 | 6 | B |
| LEU | CD2 | 67 | 49.1 | 5.3 | 36.9 | 4 | B |
| LEU | C | 67 | 49.7 | 9.6 | 37.0 | 6 | B |
| LEU | O | 67 | 49.3 | 10.6 | 37.6 | 6 | B |
| ASN | N | 68 | 50.1 | 9.6 | 35.8 | 9 | B |
| ASN | CA | 68 | 50.0 | 10.8 | 34.9 | 9 | B |
| ASN | CB | 68 | 51.1 | 10.8 | 33.9 | 9 | B |
| ASN | CG | 68 | 52.4 | 11.2 | 34.5 | 6 | B |
| ASN | OD1 | 68 | 53.5 | 11.1 | 33.9 | 2 | B |
| ASN | ND2 | 68 | 52.4 | 11.7 | 35.7 | 10 | B |
| ASN | C | 68 | 48.6 | 10.8 | 34.2 | 9 | B |
| ASN | O | 68 | 48.4 | 9.9 | 33.3 | 9 | B |
| ILE | N | 69 | 47.8 | 11.8 | 34.5 | 8 | B |
| ILE | CA | 69 | 46.5 | 11.9 | 33.9 | 9 | B |
| ILE | CB | 69 | 45.4 | 12.1 | 34.9 | 7 | B |
| ILE | CG2 | 69 | 44.0 | 12.1 | 34.3 | 8 | B |
| ILE | CG1 | 69 | 45.5 | 11.1 | 36.0 | 5 | B |
| ILE | CD1 | 69 | 45.4 | 9.6 | 35.5 | 6 | B |
| ILE | C | 69 | 46.3 | 13.0 | 32.8 | 10 | B |
| ILE | O | 69 | 46.8 | 14.1 | 33.0 | 11 | B |
| LEU | N | 70 | 45.7 | 12.6 | 31.7 | 9 | B |
| LEU | CA | 70 | 45.5 | 13.5 | 30.6 | 7 | B |
| LEU | CB | 70 | 44.9 | 12.7 | 29.4 | 5 | B |
| LEU | CG | 70 | 44.8 | 13.3 | 28.0 | 5 | B |
| LEU | CD1 | 70 | 46.1 | 13.1 | 27.2 | 5 | B |
| LEU | CD2 | 70 | 43.6 | 12.8 | 27.3 | 3 | B |
| LEU | C | 70 | 44.5 | 14.6 | 30.9 | 7 | B |
| LEU | O | 70 | 43.3 | 14.3 | 31.3 | 7 | B |
| VAL | N | 71 | 44.8 | 15.9 | 30.8 | 3 | B |
| VAL | CA | 71 | 43.9 | 16.9 | 31.1 | 1 | B |
| VAL | CB | 71 | 44.6 | 18.2 | 31.4 | 1 | B |
| VAL | CG1 | 71 | 43.7 | 19.2 | 31.9 | 4 | B |
| VAL | CG2 | 71 | 45.7 | 18.0 | 32.3 | 1 | B |
| VAL | C | 71 | 43.0 | 17.1 | 29.8 | 1 | B |
| VAL | O | 71 | 43.5 | 17.6 | 28.8 | 4 | B |
| ASP | N | 72 | 41.8 | 16.7 | 29.9 | 1 | B |
| ASP | CA | 72 | 40.9 | 16.7 | 28.8 | 1 | B |
| ASP | CB | 72 | 40.5 | 15.3 | 28.3 | 3 | B |
| ASP | CG | 72 | 39.5 | 15.2 | 27.2 | 4 | B |
| ASP | OD1 | 72 | 39.3 | 16.2 | 26.5 | 6 | B |
| ASP | OD2 | 72 | 38.8 | 14.2 | 27.1 | 3 | B |
| ASP | C | 72 | 39.6 | 17.5 | 29.1 | 1 | B |
| ASP | O | 72 | 38.7 | 16.9 | 29.8 | 4 | B |
| THR | N | 73 | 39.4 | 18.7 | 28.6 | 1 | B |
| THR | CA | 73 | 38.2 | 19.4 | 28.8 | 1 | B |
| THR | CB | 73 | 38.5 | 20.9 | 28.7 | 1 | B |
| THR | OG1 | 73 | 39.1 | 21.2 | 27.5 | 1 | B |
| THR | CG2 | 73 | 39.4 | 21.4 | 29.8 | 1 | B |
| THR | C | 73 | 37.1 | 19.1 | 27.8 | 1 | B |
| THR | O | 73 | 36.1 | 19.8 | 27.6 | 2 | B |
| GLY | N | 74 | 37.2 | 17.9 | 27.2 | 1 | B |
| GLY | CA | 74 | 36.2 | 17.4 | 26.3 | 1 | B |
| GLY | C | 74 | 35.4 | 16.2 | 26.7 | 1 | B |
| GLY | O | 74 | 34.7 | 15.6 | 25.9 | 1 | B |
| SER | N | 75 | 35.6 | 15.8 | 27.9 | 1 | B |
| SER | CA | 75 | 34.8 | 14.7 | 28.5 | 1 | B |
| SER | CB | 75 | 35.6 | 13.4 | 28.2 | 4 | B |
| SER | OG | 75 | 36.8 | 13.3 | 29.0 | 4 | B |
| SER | C | 75 | 34.6 | 14.9 | 30.0 | 1 | B |
| SER | O | 75 | 35.2 | 15.8 | 30.5 | 1 | B |
| SER | N | 76 | 33.9 | 14.0 | 30.6 | 1 | B |
| SER | CA | 76 | 33.7 | 14.1 | 32.0 | 1 | B |
| SER | CB | 76 | 32.3 | 14.7 | 32.3 | 1 | B |
| SER | OG | 76 | 32.3 | 16.1 | 32.0 | 3 | B |
| SER | C | 76 | 33.9 | 12.9 | 32.8 | 1 | B |
| SER | O | 76 | 33.3 | 12.7 | 33.9 | 2 | B |
| ASN | N | 77 | 34.8 | 12.0 | 32.4 | 1 | B |
| ASN | CA | 77 | 35.1 | 10.8 | 33.2 | 2 | B |
| ASN | CB | 77 | 34.8 | 9.6 | 32.4 | 5 | B |
| ASN | CG | 77 | 33.3 | 9.4 | 32.1 | 5 | B |
| ASN | OD1 | 77 | 32.8 | 10.1 | 31.2 | 8 | B |
| ASN | ND2 | 77 | 32.7 | 8.5 | 32.8 | 5 | B |
| ASN | C | 77 | 36.5 | 10.9 | 33.6 | 5 | B |
| ASN | O | 77 | 37.4 | 11.3 | 32.9 | 4 | B |
| PHE | N | 78 | 36.7 | 10.4 | 34.8 | 5 | B |
| PHE | CA | 78 | 38.1 | 10.3 | 35.4 | 3 | B |
| PHE | CB | 78 | 38.1 | 10.8 | 36.9 | 1 | B |
| PHE | CG | 78 | 39.4 | 10.6 | 37.5 | 2 | B |
| PHE | CD1 | 78 | 39.5 | 10.5 | 38.9 | 3 | B |
| PHE | CD2 | 78 | 40.6 | 10.4 | 36.8 | 3 | B |
| PHE | CE1 | 78 | 40.7 | 10.4 | 39.6 | 2 | B |
| PHE | CE2 | 78 | 41.8 | 10.3 | 37.4 | 3 | B |
| PHE | CZ | 78 | 41.9 | 10.2 | 38.8 | 2 | B |
| PHE | C | 78 | 38.3 | 8.8 | 35.3 | 5 | B |
| PHE | O | 78 | 37.6 | 8.0 | 36.0 | 7 | B |
| ALA | N | 79 | 39.2 | 8.4 | 34.4 | 6 | B |
| ALA | CA | 79 | 39.5 | 7.0 | 34.2 | 6 | B |
| ALA | CB | 79 | 38.8 | 6.5 | 32.9 | 3 | B |
| ALA | C | 79 | 41.0 | 6.8 | 34.1 | 7 | B |
| ALA | O | 79 | 41.7 | 7.7 | 33.9 | 7 | B |
| VAL | N | 80 | 41.4 | 5.6 | 34.4 | 7 | B |
| VAL | CA | 80 | 42.8 | 5.2 | 34.4 | 9 | B |
| VAL | CB | 80 | 43.5 | 5.5 | 35.8 | 10 | B |
| VAL | CG1 | 80 | 43.2 | 6.9 | 36.3 | 12 | B |
| VAL | CG2 | 80 | 43.0 | 4.5 | 36.8 | 11 | B |
| VAL | C | 80 | 43.0 | 3.8 | 34.0 | 8 | B |
| VAL | O | 80 | 42.1 | 3.0 | 34.2 | 7 | B |
| GLY | N | 81 | 44.2 | 3.4 | 33.6 | 8 | B |
| GLY | CA | 81 | 44.5 | 2.0 | 33.2 | 10 | B |
| GLY | C | 81 | 44.5 | 1.2 | 34.5 | 12 | B |
| GLY | O | 81 | 45.1 | 1.6 | 35.5 | 14 | B |
| ALA | N | 82 | 43.8 | 0.1 | 34.5 | 15 | B |
| ALA | CA | 82 | 43.8 | −0.8 | 35.6 | 17 | B |
| ALA | CB | 82 | 42.4 | −0.8 | 36.2 | 16 | B |
| ALA | C | 82 | 44.2 | −2.2 | 35.4 | 20 | B |
| ALA | O | 82 | 43.8 | −3.2 | 36.0 | 24 | B |
| ALA | N | 83 | 45.2 | −2.4 | 34.5 | 21 | B |
| ALA | CA | 83 | 45.7 | −3.7 | 34.1 | 20 | B |
| ALA | CB | 83 | 44.6 | −4.7 | 33.7 | 21 | B |
| ALA | C | 83 | 46.7 | −3.6 | 33.0 | 22 | B |
| ALA | O | 83 | 46.5 | −2.9 | 32.0 | 21 | B |
| PRO | N | 84 | 47.9 | −4.2 | 33.1 | 23 | B |
| PRO | CD | 84 | 48.0 | −5.5 | 33.9 | 24 | B |
| PRO | CA | 84 | 49.0 | −4.2 | 32.1 | 23 | B |
| PRO | CB | 84 | 49.8 | −5.4 | 32.4 | 27 | B |
| PRO | CG | 84 | 48.8 | −6.4 | 33.0 | 24 | B |
| PRO | C | 84 | 48.5 | −4.1 | 30.7 | 22 | B |
| PRO | O | 84 | 47.6 | −4.8 | 30.2 | 17 | B |
| HIS | N | 85 | 49.1 | −3.2 | 29.9 | 22 | B |
| HIS | CA | 85 | 48.8 | −3.0 | 28.5 | 21 | B |
| HIS | CB | 85 | 47.8 | −1.8 | 28.4 | 20 | B |
| HIS | CG | 85 | 47.6 | −1.4 | 27.0 | 22 | B |
| HIS | CD2 | 85 | 46.4 | −1.4 | 26.2 | 22 | B |
| HIS | ND1 | 85 | 48.6 | −1.0 | 26.1 | 22 | B |
| HIS | CE1 | 85 | 48.0 | −0.7 | 24.9 | 22 | B |
| HIS | NE2 | 85 | 46.7 | −1.0 | 25.0 | 21 | B |
| HIS | C | 85 | 50.1 | −2.6 | 27.8 | 21 | B |
| HIS | O | 85 | 51.0 | −1.9 | 28.3 | 19 | B |
| PRO | N | 86 | 50.3 | −3.2 | 26.6 | 20 | B |
| PRO | CD | 86 | 49.3 | −4.1 | 25.9 | 19 | B |
| PRO | CA | 86 | 51.5 | −3.1 | 25.8 | 19 | B |
| PRO | CB | 86 | 51.0 | −3.4 | 24.4 | 18 | B |
| PRO | CG | 86 | 50.1 | −4.5 | 24.7 | 19 | B |
| PRO | C | 86 | 52.2 | −1.7 | 25.9 | 20 | B |
| PRO | O | 86 | 53.4 | −1.6 | 25.7 | 22 | B |
| PHE | N | 87 | 51.4 | −0.7 | 26.1 | 20 | B |
| PHE | CA | 87 | 52.0 | 0.7 | 26.1 | 20 | B |
| PHE | CB | 87 | 51.2 | 1.6 | 25.3 | 18 | B |
| PHE | CG | 87 | 51.2 | 1.3 | 23.8 | 23 | B |
| PHE | CD1 | 87 | 50.4 | 1.9 | 22.9 | 25 | B |
| PHE | CD2 | 87 | 52.1 | 0.3 | 23.3 | 25 | B |
| PHE | CE1 | 87 | 50.5 | 1.6 | 21.5 | 24 | B |
| PHE | CE2 | 87 | 52.2 | 0.0 | 22.0 | 28 | B |
| PHE | CZ | 87 | 51.4 | 0.6 | 21.1 | 25 | B |
| PHE | C | 87 | 52.1 | 1.3 | 27.6 | 21 | B |
| PHE | O | 87 | 53.1 | 2.0 | 27.9 | 24 | B |
| LEU | N | 88 | 51.2 | 0.9 | 28.4 | 20 | B |
| LEU | CA | 88 | 51.2 | 1.4 | 29.8 | 19 | B |
| LEU | CB | 88 | 50.0 | 0.9 | 30.5 | 24 | B |
| LEU | CG | 88 | 48.6 | 1.6 | 30.2 | 22 | B |
| LEU | CD1 | 88 | 47.5 | 1.1 | 31.1 | 24 | B |
| LEU | CD2 | 88 | 48.8 | 3.1 | 30.4 | 21 | B |
| LEU | C | 88 | 52.5 | 1.0 | 30.6 | 19 | B |
| LEU | O | 88 | 52.8 | −0.2 | 30.8 | 16 | B |
| HIS | N | 89 | 53.2 | 2.0 | 31.0 | 19 | B |
| HIS | CA | 89 | 54.4 | 1.8 | 31.8 | 21 | B |
| HIS | CB | 89 | 55.2 | 3.1 | 31.9 | 24 | B |
| HIS | CG | 89 | 55.8 | 3.6 | 30.7 | 32 | B |
| HIS | CD2 | 89 | 55.9 | 4.8 | 30.1 | 33 | B |
| HIS | ND1 | 89 | 56.6 | 2.7 | 29.9 | 36 | B |
| HIS | CE1 | 89 | 57.1 | 3.4 | 28.9 | 36 | B |
| HIS | NE2 | 89 | 56.6 | 4.7 | 28.9 | 36 | B |
| HIS | C | 89 | 54.0 | 1.3 | 33.2 | 19 | B |
| HIS | O | 89 | 54.8 | 0.6 | 33.8 | 19 | B |
| ARG | N | 90 | 52.9 | 1.8 | 33.7 | 18 | B |
| ARG | CA | 90 | 52.3 | 1.4 | 35.0 | 18 | B |
| ARG | CB | 90 | 52.9 | 2.3 | 36.1 | 16 | B |
| ARG | CG | 90 | 52.7 | 3.8 | 35.9 | 18 | B |
| ARG | CD | 90 | 53.2 | 4.5 | 37.1 | 26 | B |
| ARG | NE | 90 | 53.3 | 6.0 | 36.9 | 27 | B |
| ARG | CZ | 90 | 53.7 | 6.8 | 37.8 | 27 | B |
| ARG | NH1 | 90 | 54.1 | 6.4 | 39.0 | 29 | B |
| ARG | NH2 | 90 | 53.8 | 8.1 | 37.6 | 26 | B |
| ARG | C | 90 | 50.8 | 1.5 | 34.9 | 18 | B |
| ARG | O | 90 | 50.3 | 2.0 | 34.0 | 16 | B |
| TYR | N | 91 | 50.2 | 1.0 | 36.0 | 17 | B |
| TYR | CA | 91 | 48.7 | 1.1 | 36.1 | 16 | B |
| TYR | CB | 91 | 48.0 | −0.1 | 35.3 | 15 | B |
| TYR | CG | 91 | 48.3 | −1.4 | 35.9 | 19 | B |
| TYR | CD1 | 91 | 47.6 | −1.9 | 36.9 | 22 | B |
| TYR | CE1 | 91 | 47.8 | −3.2 | 37.4 | 27 | B |
| TYR | CD2 | 91 | 49.3 | −2.2 | 35.3 | 22 | B |
| TYR | CE2 | 91 | 49.6 | −3.5 | 35.8 | 26 | B |
| TYR | CZ | 91 | 48.8 | −4.0 | 36.9 | 28 | B |
| TYR | OH | 91 | 49.0 | −5.3 | 37.4 | 28 | B |
| TYR | C | 91 | 48.2 | 1.2 | 37.5 | 12 | B |
| TYR | O | 91 | 48.9 | 1.2 | 38.5 | 13 | B |
| TYR | N | 92 | 46.8 | 1.3 | 37.6 | 10 | B |
| TYR | CA | 92 | 46.2 | 1.4 | 38.9 | 7 | B |
| TYR | CB | 92 | 44.9 | 2.3 | 38.8 | 6 | B |
| TYR | CG | 92 | 44.0 | 2.4 | 40.0 | 6 | B |
| TYR | CD1 | 92 | 44.5 | 2.5 | 41.3 | 7 | B |
| TYR | CE1 | 92 | 43.7 | 2.7 | 42.4 | 5 | B |
| TYR | CD2 | 92 | 42.6 | 2.4 | 39.8 | 5 | B |
| TYR | CE2 | 92 | 41.8 | 2.5 | 40.9 | 5 | B |
| TYR | CZ | 92 | 42.3 | 2.6 | 42.2 | 4 | B |
| TYR | OH | 92 | 41.5 | 2.8 | 43.3 | 5 | B |
| TYR | C | 92 | 45.9 | 0.1 | 39.6 | 8 | B |
| TYR | O | 92 | 45.0 | −0.7 | 39.1 | 8 | B |
| GLN | N | 93 | 46.6 | −0.2 | 40.6 | 8 | B |
| GLN | CA | 93 | 46.3 | −1.5 | 41.3 | 9 | B |
| GLN | CB | 93 | 47.6 | −2.0 | 42.0 | 15 | B |
| GLN | CG | 93 | 48.7 | −2.4 | 40.9 | 17 | B |
| GLN | CD | 93 | 50.0 | −2.7 | 41.6 | 20 | B |
| GLN | OE1 | 93 | 50.6 | −1.9 | 42.3 | 23 | B |
| GLN | NE2 | 93 | 50.5 | −3.9 | 41.3 | 22 | B |
| GLN | C | 93 | 45.3 | −1.3 | 42.4 | 11 | B |
| GLN | O | 93 | 45.6 | −0.7 | 43.4 | 12 | B |
| ARG | N | 94 | 44.1 | −1.7 | 42.1 | 12 | B |
| ARG | CA | 94 | 43.0 | −1.6 | 43.1 | 14 | B |
| ARG | CB | 94 | 41.6 | −2.0 | 42.4 | 11 | B |
| ARG | CG | 94 | 41.2 | −1.0 | 41.3 | 6 | B |
| ARG | CD | 94 | 40.0 | −1.5 | 40.6 | 10 | B |
| ARG | NE | 94 | 40.3 | −2.4 | 39.4 | 7 | B |
| ARG | CZ | 94 | 39.4 | −2.9 | 38.7 | 11 | B |
| ARG | NH1 | 94 | 38.1 | −2.8 | 38.9 | 14 | B |
| ARG | NH2 | 94 | 39.8 | −3.7 | 37.6 | 9 | B |
| ARG | C | 94 | 43.2 | −2.4 | 44.3 | 14 | B |
| ARG | O | 94 | 43.1 | −2.0 | 45.5 | 11 | B |
| GLN | N | 95 | 43.5 | −3.7 | 44.1 | 15 | B |
| GLN | CA | 95 | 43.7 | −4.6 | 45.2 | 15 | B |
| GLN | CB | 95 | 44.2 | −6.0 | 44.7 | 19 | B |
| GLN | CG | 95 | 43.3 | −6.7 | 43.8 | 24 | B |
| GLN | CD | 95 | 43.8 | −8.1 | 43.4 | 31 | B |
| GLN | OE1 | 95 | 43.8 | −9.0 | 44.2 | 33 | B |
| GLN | NE2 | 95 | 44.1 | −8.2 | 42.1 | 33 | B |
| GLN | C | 95 | 44.7 | −4.1 | 46.3 | 15 | B |
| GLN | O | 95 | 44.6 | −4.5 | 47.5 | 15 | B |
| LEU | N | 96 | 45.6 | −3.2 | 45.9 | 13 | B |
| LEU | CA | 96 | 46.6 | −2.6 | 46.8 | 14 | B |
| LEU | CB | 96 | 47.9 | −2.4 | 46.1 | 12 | B |
| LEU | CG | 96 | 48.7 | −3.6 | 45.9 | 10 | B |
| LEU | CD1 | 96 | 49.8 | −3.4 | 44.9 | 5 | B |
| LEU | CD2 | 96 | 49.4 | −4.0 | 47.3 | 14 | B |
| LEU | C | 96 | 46.1 | −1.4 | 47.5 | 13 | B |
| LEU | O | 96 | 46.8 | −0.8 | 48.3 | 16 | B |
| SER | N | 97 | 44.9 | −1.0 | 47.3 | 15 | B |
| SER | CA | 97 | 44.3 | 0.2 | 48.0 | 17 | B |
| SER | CB | 97 | 43.6 | 1.1 | 47.0 | 16 | B |
| SER | OG | 97 | 43.0 | 2.2 | 47.6 | 20 | B |
| SER | C | 97 | 43.3 | −0.2 | 49.1 | 19 | B |
| SER | O | 97 | 42.5 | −1.1 | 48.9 | 23 | B |
| SER | N | 98 | 43.4 | 0.5 | 50.2 | 17 | B |
| SER | CA | 98 | 42.5 | 0.3 | 51.3 | 14 | B |
| SER | CB | 98 | 43.2 | 0.6 | 52.6 | 17 | B |
| SER | OG | 98 | 43.7 | 1.9 | 52.6 | 20 | B |
| SER | C | 98 | 41.2 | 1.1 | 51.1 | 12 | B |
| SER | O | 98 | 40.1 | 0.6 | 51.5 | 14 | B |
| THR | N | 99 | 41.3 | 2.3 | 50.6 | 8 | B |
| THR | CA | 99 | 40.2 | 3.2 | 50.4 | 5 | B |
| THR | CB | 99 | 40.6 | 4.6 | 50.5 | 1 | B |
| THR | OG1 | 99 | 41.7 | 4.8 | 49.5 | 1 | B |
| THR | CG2 | 99 | 41.1 | 5.0 | 51.9 | 7 | B |
| THR | C | 99 | 39.5 | 3.0 | 49.1 | 7 | B |
| THR | O | 99 | 38.7 | 3.8 | 48.8 | 11 | B |
| TYR | N | 100 | 39.7 | 1.9 | 48.4 | 7 | B |
| TYR | CA | 100 | 39.0 | 1.6 | 47.2 | 3 | B |
| TYR | CB | 100 | 39.7 | 0.5 | 46.4 | 6 | B |
| TYR | CG | 100 | 39.1 | 0.1 | 45.1 | 7 | B |
| TYR | CD1 | 100 | 39.1 | 0.9 | 44.0 | 9 | B |
| TYR | CE1 | 100 | 38.5 | 0.5 | 42.8 | 13 | B |
| TYR | CD2 | 100 | 38.5 | −1.2 | 45.0 | 6 | B |
| TYR | CE2 | 100 | 37.9 | −1.6 | 43.8 | 12 | B |
| TYR | CZ | 100 | 37.9 | −0.7 | 42.7 | 13 | B |
| TYR | OH | 100 | 37.3 | −1.1 | 41.5 | 16 | B |
| TYR | C | 100 | 37.6 | 1.1 | 47.4 | 2 | B |
| TYR | O | 100 | 37.2 | 0.6 | 48.5 | 2 | B |
| ARG | N | 101 | 36.7 | 1.3 | 46.4 | 1 | B |
| ARG | CA | 101 | 35.4 | 0.9 | 46.5 | 3 | B |
| ARG | CB | 101 | 34.4 | 2.0 | 47.0 | 5 | B |
| ARG | CG | 101 | 34.3 | 2.1 | 48.5 | 9 | B |
| ARG | CD | 101 | 33.1 | 3.1 | 48.8 | 12 | B |
| ARG | NE | 101 | 32.9 | 3.2 | 50.3 | 6 | B |
| ARG | CZ | 101 | 33.8 | 3.5 | 51.2 | 4 | B |
| ARG | NH1 | 101 | 35.0 | 3.7 | 50.8 | 5 | B |
| ARG | NH2 | 101 | 33.4 | 3.6 | 52.4 | 6 | B |
| ARG | C | 101 | 35.0 | 0.4 | 45.1 | 5 | B |
| ARG | O | 101 | 35.8 | 0.6 | 44.2 | 7 | B |
| ASP | N | 102 | 33.8 | −0.2 | 45.0 | 2 | B |
| ASP | CA | 102 | 33.5 | −0.7 | 43.6 | 2 | B |
| ASP | CB | 102 | 34.0 | −2.2 | 43.5 | 1 | B |
| ASP | CG | 102 | 33.3 | −3.0 | 42.4 | 4 | B |
| ASP | OD1 | 102 | 33.4 | −2.6 | 41.3 | 8 | B |
| ASP | OD2 | 102 | 32.8 | −4.1 | 42.8 | 8 | B |
| ASP | C | 102 | 31.9 | −0.7 | 43.5 | 4 | B |
| ASP | O | 102 | 31.2 | −1.5 | 44.1 | 8 | B |
| LEU | N | 103 | 31.4 | 0.3 | 42.7 | 3 | B |
| LEU | CA | 103 | 30.0 | 0.4 | 42.5 | 2 | B |
| LEU | CB | 103 | 29.7 | 1.8 | 41.9 | 1 | B |
| LEU | CG | 103 | 30.5 | 3.0 | 42.3 | 1 | B |
| LEU | CD1 | 103 | 31.4 | 3.4 | 41.2 | 1 | B |
| LEU | CD2 | 103 | 29.6 | 4.1 | 42.7 | 1 | B |
| LEU | C | 103 | 29.4 | −0.7 | 41.7 | 4 | B |
| LEU | O | 103 | 28.2 | −0.5 | 41.2 | 4 | B |
| ARG | N | 104 | 30.1 | −1.8 | 41.5 | 6 | B |
| ARG | CA | 104 | 29.6 | −2.9 | 40.7 | 9 | B |
| ARG | CB | 104 | 28.7 | −3.8 | 41.6 | 13 | B |
| ARG | CG | 104 | 29.5 | −4.7 | 42.6 | 17 | B |
| ARG | CD | 104 | 28.5 | −5.3 | 43.6 | 27 | B |
| ARG | NE | 104 | 27.2 | −5.8 | 43.1 | 32 | B |
| ARG | CZ | 104 | 26.1 | −5.2 | 43.3 | 34 | B |
| ARG | NH1 | 104 | 26.0 | −4.1 | 44.0 | 30 | B |
| ARG | NH2 | 104 | 25.0 | −5.8 | 42.8 | 36 | B |
| ARG | C | 104 | 28.8 | −2.5 | 39.5 | 9 | B |
| ARG | O | 104 | 27.6 | −2.8 | 39.4 | 10 | B |
| LYS | N | 105 | 29.4 | −1.7 | 38.6 | 10 | B |
| LYS | CA | 105 | 28.8 | −1.2 | 37.4 | 10 | B |
| LYS | CB | 105 | 27.9 | 0.0 | 37.7 | 11 | B |
| LYS | CG | 105 | 27.1 | 0.6 | 36.5 | 14 | B |
| LYS | CD | 105 | 26.1 | 1.7 | 36.9 | 21 | B |
| LYS | CE | 105 | 26.7 | 2.9 | 37.6 | 27 | B |
| LYS | NZ | 105 | 25.8 | 4.1 | 37.8 | 27 | B |
| LYS | C | 105 | 29.8 | −0.8 | 36.4 | 7 | B |
| LYS | O | 105 | 30.7 | 0.0 | 36.6 | 2 | B |
| GLY | N | 106 | 29.7 | −1.4 | 35.2 | 6 | B |
| GLY | CA | 106 | 30.7 | −1.2 | 34.1 | 8 | B |
| GLY | C | 106 | 30.4 | 0.1 | 33.4 | 6 | B |
| GLY | O | 106 | 29.4 | 0.7 | 33.6 | 7 | B |
| VAL | N | 107 | 31.4 | 0.5 | 32.5 | 4 | B |
| VAL | CA | 107 | 31.2 | 1.7 | 31.7 | 3 | B |
| VAL | CB | 107 | 31.6 | 2.9 | 32.5 | 1 | B |
| VAL | CG1 | 107 | 33.1 | 2.9 | 32.7 | 1 | B |
| VAL | CG2 | 107 | 31.1 | 4.2 | 31.8 | 4 | B |
| VAL | C | 107 | 31.9 | 1.6 | 30.4 | 4 | B |
| VAL | O | 107 | 33.0 | 1.0 | 30.4 | 1 | B |
| TYR | N | 108 | 31.4 | 2.1 | 29.4 | 8 | B |
| TYR | CA | 108 | 32.0 | 2.2 | 28.0 | 11 | B |
| TYR | CB | 108 | 31.2 | 1.3 | 27.0 | 15 | B |
| TYR | CG | 108 | 31.3 | 1.7 | 25.6 | 14 | B |
| TYR | CD1 | 108 | 32.6 | 1.8 | 25.0 | 16 | B |
| TYR | CE1 | 108 | 32.8 | 2.2 | 23.7 | 13 | B |
| TYR | CD2 | 108 | 30.3 | 2.0 | 24.8 | 16 | B |
| TYR | CE2 | 108 | 30.4 | 2.4 | 23.5 | 19 | B |
| TYR | CZ | 108 | 31.7 | 2.5 | 22.9 | 15 | B |
| TYR | OH | 108 | 31.9 | 2.9 | 21.6 | 15 | B |
| TYR | C | 108 | 32.0 | 3.6 | 27.6 | 11 | B |
| TYR | O | 108 | 31.0 | 4.2 | 27.4 | 11 | B |
| VAL | N | 109 | 33.2 | 4.1 | 27.4 | 11 | B |
| VAL | CA | 109 | 33.3 | 5.5 | 26.9 | 10 | B |
| VAL | CB | 109 | 34.1 | 6.4 | 28.0 | 7 | B |
| VAL | CG1 | 109 | 34.3 | 7.7 | 27.4 | 4 | B |
| VAL | CG2 | 109 | 33.2 | 6.4 | 29.2 | 4 | B |
| VAL | C | 109 | 34.1 | 5.6 | 25.6 | 12 | B |
| VAL | O | 109 | 35.3 | 5.4 | 25.5 | 11 | B |
| PRO | N | 110 | 33.4 | 6.0 | 24.5 | 13 | B |
| PRO | CD | 110 | 31.9 | 6.0 | 24.4 | 14 | B |
| PRO | CA | 110 | 34.0 | 6.2 | 23.2 | 13 | B |
| PRO | CB | 110 | 32.9 | 5.7 | 22.3 | 10 | B |
| PRO | CG | 110 | 31.7 | 6.3 | 22.9 | 9 | B |
| PRO | C | 110 | 34.4 | 7.6 | 23.0 | 14 | B |
| PRO | O | 110 | 33.9 | 8.5 | 23.6 | 12 | B |
| TYR | N | 111 | 35.3 | 7.8 | 22.0 | 15 | B |
| TYR | CA | 111 | 35.7 | 9.2 | 21.6 | 13 | B |
| TYR | CB | 111 | 37.1 | 9.4 | 22.3 | 12 | B |
| TYR | CG | 111 | 37.1 | 9.3 | 23.8 | 8 | B |
| TYR | CD1 | 111 | 36.8 | 10.4 | 24.6 | 10 | B |
| TYR | CE1 | 111 | 36.9 | 10.4 | 26.0 | 13 | B |
| TYR | CD2 | 111 | 37.5 | 8.2 | 24.4 | 11 | B |
| TYR | CE2 | 111 | 37.5 | 8.1 | 25.8 | 13 | B |
| TYR | CZ | 111 | 37.2 | 9.2 | 26.6 | 12 | B |
| TYR | OH | 111 | 37.3 | 9.1 | 28.0 | 9 | B |
| TYR | C | 111 | 35.9 | 9.2 | 20.1 | 15 | B |
| TYR | O | 111 | 35.6 | 8.2 | 19.4 | 14 | B |
| THR | N | 112 | 36.2 | 10.4 | 19.6 | 17 | B |
| THR | CA | 112 | 36.4 | 10.6 | 18.2 | 18 | B |
| THR | CB | 112 | 37.0 | 12.1 | 17.9 | 16 | B |
| THR | OG1 | 112 | 35.9 | 13.0 | 18.0 | 15 | B |
| THR | CG2 | 112 | 37.6 | 12.1 | 16.5 | 15 | B |
| THR | C | 112 | 37.4 | 9.6 | 17.7 | 18 | B |
| THR | O | 112 | 37.2 | 8.9 | 16.8 | 14 | B |
| GLN | N | 113 | 38.5 | 9.6 | 18.5 | 21 | B |
| GLN | CA | 113 | 39.6 | 8.7 | 18.2 | 23 | B |
| GLN | CB | 113 | 40.9 | 9.4 | 17.8 | 29 | B |
| GLN | CG | 113 | 40.9 | 10.0 | 16.3 | 33 | B |
| GLN | CD | 113 | 41.0 | 8.9 | 15.3 | 34 | B |
| GLN | OE1 | 113 | 41.9 | 8.1 | 15.3 | 33 | B |
| GLN | NE2 | 113 | 40.1 | 9.0 | 14.3 | 35 | B |
| GLN | C | 113 | 39.9 | 8.0 | 19.6 | 19 | B |
| GLN | O | 113 | 40.5 | 8.6 | 20.5 | 18 | B |
| GLY | N | 114 | 39.3 | 6.8 | 19.7 | 18 | B |
| GLY | CA | 114 | 39.5 | 6.1 | 21.0 | 19 | B |
| GLY | C | 114 | 38.3 | 5.5 | 21.7 | 18 | B |
| GLY | O | 114 | 37.2 | 6.0 | 21.4 | 17 | B |
| LYS | N | 115 | 38.5 | 4.5 | 22.5 | 18 | B |
| LYS | CA | 115 | 37.4 | 3.9 | 23.3 | 20 | B |
| LYS | CB | 115 | 36.4 | 3.2 | 22.3 | 22 | B |
| LYS | CG | 115 | 37.1 | 2.1 | 21.4 | 30 | B |
| LYS | CD | 115 | 36.1 | 1.1 | 20.9 | 33 | B |
| LYS | CE | 115 | 35.0 | 1.7 | 19.9 | 35 | B |
| LYS | NZ | 115 | 34.1 | 0.7 | 19.4 | 35 | B |
| LYS | C | 115 | 37.9 | 2.9 | 24.3 | 18 | B |
| LYS | O | 115 | 38.9 | 2.2 | 24.1 | 17 | B |
| TRP | N | 116 | 37.3 | 2.9 | 25.4 | 15 | B |
| TRP | CA | 116 | 37.6 | 2.0 | 26.5 | 13 | B |
| TRP | CB | 116 | 38.7 | 2.6 | 27.4 | 11 | B |
| TRP | CG | 116 | 38.4 | 4.0 | 27.9 | 9 | B |
| TRP | CD2 | 116 | 37.5 | 4.3 | 28.9 | 6 | B |
| TRP | CE2 | 116 | 37.6 | 5.7 | 29.1 | 6 | B |
| TRP | CE3 | 116 | 36.6 | 3.6 | 29.7 | 2 | B |
| TRP | CD1 | 116 | 39.0 | 5.1 | 27.5 | 7 | B |
| TRP | NE1 | 116 | 38.5 | 6.2 | 28.2 | 4 | B |
| TRP | CZ2 | 116 | 36.8 | 6.4 | 30.0 | 1 | B |
| TRP | CZ3 | 116 | 35.9 | 4.3 | 30.7 | 1 | B |
| TRP | CH2 | 116 | 36.0 | 5.7 | 30.8 | 1 | B |
| TRP | C | 116 | 36.4 | 1.6 | 27.3 | 13 | B |
| TRP | O | 116 | 35.5 | 2.4 | 27.5 | 13 | B |
| GLU | N | 117 | 36.5 | 0.4 | 27.9 | 15 | B |
| GLU | CA | 117 | 35.5 | −0.2 | 28.7 | 13 | B |
| GLU | CB | 117 | 35.0 | −1.5 | 28.1 | 16 | B |
| GLU | CG | 117 | 34.8 | −2.7 | 29.0 | 28 | B |
| GLU | CD | 117 | 33.6 | −2.7 | 29.9 | 37 | B |
| GLU | OE1 | 117 | 32.5 | −2.6 | 29.3 | 42 | B |
| GLU | OE2 | 117 | 33.7 | −2.9 | 31.1 | 41 | B |
| GLU | C | 117 | 36.1 | −0.5 | 30.1 | 12 | B |
| GLU | O | 117 | 37.2 | −1.0 | 30.1 | 13 | B |
| GLY | N | 118 | 35.4 | −0.1 | 31.2 | 11 | B |
| GLY | CA | 118 | 36.0 | −0.3 | 32.5 | 12 | B |
| GLY | C | 118 | 35.1 | −0.7 | 33.6 | 15 | B |
| GLY | O | 118 | 33.9 | −0.9 | 33.4 | 15 | B |
| GLU | N | 119 | 35.6 | −0.7 | 34.8 | 16 | B |
| GLU | CA | 119 | 34.8 | −1.0 | 36.0 | 18 | B |
| GLU | CB | 119 | 35.3 | −2.3 | 36.7 | 21 | B |
| GLU | CG | 119 | 35.3 | −3.5 | 35.7 | 28 | B |
| GLU | CD | 119 | 36.1 | −4.7 | 36.2 | 30 | B |
| GLU | OE1 | 119 | 36.1 | −5.8 | 35.5 | 28 | B |
| GLU | OE2 | 119 | 36.8 | −4.6 | 37.3 | 33 | B |
| GLU | C | 119 | 34.7 | 0.2 | 37.0 | 15 | B |
| GLU | O | 119 | 35.7 | 0.6 | 37.5 | 15 | B |
| LEU | N | 120 | 33.5 | 0.7 | 37.2 | 15 | B |
| LEU | CA | 120 | 33.3 | 1.8 | 38.1 | 14 | B |
| LEU | CB | 120 | 31.9 | 2.4 | 37.9 | 9 | B |
| LEU | CG | 120 | 31.6 | 3.1 | 36.6 | 8 | B |
| LEU | CD1 | 120 | 30.1 | 3.5 | 36.5 | 7 | B |
| LEU | CD2 | 120 | 32.5 | 4.3 | 36.4 | 9 | B |
| LEU | C | 120 | 33.5 | 1.6 | 39.6 | 15 | B |
| LEU | O | 120 | 33.0 | 0.6 | 40.1 | 17 | B |
| GLY | N | 121 | 34.3 | 2.4 | 40.2 | 13 | B |
| GLY | CA | 121 | 34.5 | 2.3 | 41.6 | 12 | B |
| GLY | C | 121 | 34.8 | 3.7 | 42.1 | 12 | B |
| GLY | O | 121 | 34.4 | 4.7 | 41.5 | 13 | B |
| THR | N | 122 | 35.4 | 3.9 | 43.3 | 11 | B |
| THR | CA | 122 | 35.7 | 5.2 | 43.9 | 11 | B |
| THR | CB | 122 | 34.5 | 5.7 | 44.8 | 9 | B |
| THR | OG1 | 122 | 34.5 | 5.1 | 46.1 | 10 | B |
| THR | CG2 | 122 | 33.2 | 5.5 | 44.2 | 6 | B |
| THR | C | 122 | 37.0 | 5.0 | 44.7 | 12 | B |
| THR | O | 122 | 37.5 | 3.9 | 44.9 | 14 | B |
| ASP | N | 123 | 37.5 | 6.2 | 45.1 | 12 | B |
| ASP | CA | 123 | 38.7 | 6.2 | 45.9 | 9 | B |
| ASP | CB | 123 | 39.8 | 5.5 | 45.1 | 11 | B |
| ASP | CG | 123 | 40.8 | 4.8 | 46.0 | 11 | B |
| ASP | OD1 | 123 | 41.3 | 5.4 | 47.0 | 16 | B |
| ASP | OD2 | 123 | 41.1 | 3.6 | 45.7 | 10 | B |
| ASP | C | 123 | 39.2 | 7.6 | 46.2 | 8 | B |
| ASP | O | 123 | 38.5 | 8.6 | 45.8 | 9 | B |
| LEU | N | 124 | 40.2 | 7.7 | 47.0 | 6 | B |
| LEU | CA | 124 | 40.8 | 9.0 | 47.3 | 6 | B |
| LEU | CB | 124 | 41.5 | 9.0 | 48.7 | 9 | B |
| LEU | CG | 124 | 40.6 | 8.6 | 49.9 | 9 | B |
| LEU | CD1 | 124 | 41.5 | 8.5 | 51.2 | 10 | B |
| LEU | CD2 | 124 | 39.5 | 9.5 | 50.0 | 10 | B |
| LEU | C | 124 | 41.7 | 9.5 | 46.3 | 4 | B |
| LEU | O | 124 | 42.7 | 8.8 | 45.9 | 1 | B |
| VAL | N | 125 | 41.5 | 10.7 | 45.8 | 4 | B |
| VAL | CA | 125 | 42.4 | 11.3 | 44.8 | 8 | B |
| VAL | CB | 125 | 41.5 | 11.6 | 43.5 | 11 | B |
| VAL | CG1 | 125 | 42.4 | 12.3 | 42.5 | 8 | B |
| VAL | CG2 | 125 | 40.9 | 10.4 | 42.9 | 7 | B |
| VAL | C | 125 | 43.1 | 12.5 | 45.3 | 10 | B |
| VAL | O | 125 | 42.6 | 13.3 | 46.1 | 8 | B |
| SER | N | 126 | 44.3 | 12.7 | 44.7 | 11 | B |
| SER | CA | 126 | 45.1 | 13.9 | 45.1 | 13 | B |
| SER | CB | 126 | 46.1 | 13.5 | 46.2 | 11 | B |
| SER | OG | 126 | 45.6 | 13.7 | 47.5 | 13 | B |
| SER | C | 126 | 45.9 | 14.4 | 43.9 | 14 | B |
| SER | O | 126 | 46.1 | 13.7 | 42.9 | 12 | B |
| ILE | N | 127 | 46.2 | 15.7 | 44.0 | 15 | B |
| ILE | CA | 127 | 47.0 | 16.3 | 42.9 | 14 | B |
| ILE | CB | 127 | 46.2 | 17.4 | 42.2 | 15 | B |
| ILE | CG2 | 127 | 47.1 | 18.2 | 41.2 | 10 | B |
| ILE | CG1 | 127 | 45.0 | 16.8 | 41.5 | 15 | B |
| ILE | CD1 | 127 | 44.1 | 17.8 | 40.8 | 16 | B |
| ILE | C | 127 | 48.2 | 16.9 | 43.6 | 14 | B |
| ILE | O | 127 | 48.2 | 18.0 | 44.1 | 14 | B |
| PRO | N | 128 | 49.4 | 16.2 | 43.5 | 11 | B |
| PRO | CD | 128 | 49.5 | 14.9 | 42.8 | 11 | B |
| PRO | CA | 128 | 50.7 | 16.6 | 44.1 | 8 | B |
| PRO | CB | 128 | 51.7 | 15.7 | 43.4 | 7 | B |
| PRO | CG | 128 | 50.9 | 14.4 | 43.3 | 9 | B |
| PRO | C | 128 | 51.0 | 18.1 | 43.9 | 7 | B |
| PRO | O | 128 | 51.2 | 18.8 | 44.9 | 6 | B |
| HIS | N | 129 | 51.0 | 18.5 | 42.7 | 6 | B |
| HIS | CA | 129 | 51.3 | 19.9 | 42.4 | 4 | B |
| HIS | CB | 129 | 52.2 | 20.0 | 41.2 | 4 | B |
| HIS | CG | 129 | 53.5 | 19.2 | 41.3 | 4 | B |
| HIS | CD2 | 129 | 54.0 | 18.2 | 40.5 | 4 | B |
| HIS | ND1 | 129 | 54.3 | 19.2 | 42.4 | 3 | B |
| HIS | CE1 | 129 | 55.3 | 18.4 | 42.3 | 6 | B |
| HIS | NE2 | 129 | 55.1 | 17.7 | 41.2 | 10 | B |
| HIS | C | 129 | 50.0 | 20.7 | 42.1 | 3 | B |
| HIS | O | 129 | 49.9 | 21.3 | 41.1 | 1 | B |
| GLY | N | 130 | 49.1 | 20.6 | 43.1 | 1 | B |
| GLY | CA | 130 | 47.9 | 21.3 | 43.0 | 1 | B |
| GLY | C | 130 | 47.5 | 21.7 | 44.4 | 1 | B |
| GLY | O | 130 | 48.4 | 21.7 | 45.2 | 1 | B |
| PRO | N | 131 | 46.3 | 22.0 | 44.7 | 5 | B |
| PRO | CD | 131 | 45.1 | 22.1 | 43.8 | 8 | B |
| PRO | CA | 131 | 45.9 | 22.4 | 46.0 | 10 | B |
| PRO | CB | 131 | 44.4 | 22.7 | 45.9 | 10 | B |
| PRO | CG | 131 | 44.0 | 22.0 | 44.7 | 6 | B |
| PRO | C | 131 | 46.1 | 21.2 | 47.0 | 11 | B |
| PRO | O | 131 | 45.8 | 20.0 | 46.7 | 12 | B |
| ASN | N | 132 | 46.6 | 21.5 | 48.2 | 15 | B |
| ASN | CA | 132 | 46.9 | 20.5 | 49.2 | 21 | B |
| ASN | CB | 132 | 47.7 | 21.1 | 50.4 | 29 | B |
| ASN | CG | 132 | 48.2 | 20.1 | 51.3 | 34 | B |
| ASN | OD1 | 132 | 48.9 | 20.4 | 52.3 | 38 | B |
| ASN | ND2 | 132 | 47.7 | 18.8 | 51.2 | 37 | B |
| ASN | C | 132 | 45.6 | 19.9 | 49.7 | 21 | B |
| ASN | O | 132 | 45.2 | 20.2 | 50.9 | 25 | B |
| VAL | N | 133 | 44.9 | 19.0 | 48.9 | 18 | B |
| VAL | CA | 133 | 43.7 | 18.4 | 49.3 | 17 | B |
| VAL | CB | 133 | 42.4 | 19.1 | 48.9 | 16 | B |
| VAL | CG1 | 133 | 42.4 | 20.5 | 49.5 | 16 | B |
| VAL | CG2 | 133 | 42.4 | 19.2 | 47.4 | 16 | B |
| VAL | C | 133 | 43.6 | 17.0 | 48.8 | 15 | B |
| VAL | O | 133 | 44.3 | 16.6 | 47.9 | 14 | B |
| THR | N | 134 | 42.6 | 16.2 | 49.3 | 13 | B |
| THR | CA | 134 | 42.3 | 14.9 | 48.9 | 10 | B |
| THR | CB | 134 | 42.9 | 13.8 | 49.9 | 9 | B |
| THR | OG1 | 134 | 44.3 | 13.8 | 49.7 | 9 | B |
| THR | CG2 | 134 | 42.3 | 12.4 | 49.6 | 8 | B |
| THR | C | 134 | 40.8 | 14.7 | 48.9 | 11 | B |
| THR | O | 134 | 40.1 | 15.1 | 49.9 | 10 | B |
| VAL | N | 135 | 40.2 | 14.2 | 47.8 | 9 | B |
| VAL | CA | 135 | 38.8 | 14.0 | 47.7 | 8 | B |
| VAL | CB | 135 | 38.1 | 15.0 | 46.6 | 10 | B |
| VAL | CG1 | 135 | 38.3 | 16.4 | 47.0 | 15 | B |
| VAL | CG2 | 135 | 38.8 | 14.7 | 45.3 | 7 | B |
| VAL | C | 135 | 38.5 | 12.6 | 47.3 | 8 | B |
| VAL | O | 135 | 39.3 | 11.9 | 46.7 | 11 | B |
| ARG | N | 136 | 37.2 | 12.2 | 47.5 | 6 | B |
| ARG | CA | 136 | 36.8 | 10.8 | 47.1 | 4 | B |
| ARG | CB | 136 | 35.9 | 10.2 | 48.2 | 7 | B |
| ARG | CG | 136 | 35.4 | 8.8 | 47.7 | 8 | B |
| ARG | CD | 136 | 34.7 | 8.1 | 48.9 | 12 | B |
| ARG | NE | 136 | 35.7 | 7.7 | 50.0 | 14 | B |
| ARG | CZ | 136 | 36.6 | 6.8 | 49.9 | 15 | B |
| ARG | NH1 | 136 | 36.7 | 6.1 | 48.8 | 12 | B |
| ARG | NH2 | 136 | 37.4 | 6.5 | 50.9 | 16 | B |
| ARG | C | 136 | 36.1 | 11.1 | 45.9 | 5 | B |
| ARG | O | 136 | 35.1 | 11.8 | 45.9 | 7 | B |
| ALA | N | 137 | 36.6 | 10.5 | 44.8 | 4 | B |
| ALA | CA | 137 | 35.9 | 10.8 | 43.5 | 5 | B |
| ALA | CB | 137 | 36.8 | 11.6 | 42.6 | 6 | B |
| ALA | C | 137 | 35.6 | 9.5 | 42.8 | 3 | B |
| ALA | O | 137 | 36.0 | 8.4 | 43.2 | 1 | B |
| ASN | N | 138 | 34.9 | 9.5 | 41.7 | 5 | B |
| ASN | CA | 138 | 34.5 | 8.3 | 40.9 | 6 | B |
| ASN | CB | 138 | 33.3 | 8.6 | 40.1 | 7 | B |
| ASN | CG | 138 | 32.1 | 8.7 | 40.9 | 10 | B |
| ASN | OD1 | 138 | 31.8 | 8.0 | 41.9 | 10 | B |
| ASN | ND2 | 138 | 31.3 | 9.7 | 40.6 | 15 | B |
| ASN | C | 138 | 35.7 | 8.1 | 40.0 | 7 | B |
| ASN | O | 138 | 36.3 | 9.0 | 39.4 | 10 | B |
| ILE | N | 139 | 36.1 | 6.8 | 39.9 | 6 | B |
| ILE | CA | 139 | 37.2 | 6.4 | 39.1 | 3 | B |
| ILE | CB | 139 | 38.5 | 6.2 | 39.9 | 2 | B |
| ILE | CG2 | 139 | 39.7 | 6.0 | 38.9 | 1 | B |
| ILE | CG1 | 139 | 38.8 | 7.3 | 40.9 | 5 | B |
| ILE | CD1 | 139 | 40.1 | 7.1 | 41.6 | 8 | B |
| ILE | C | 139 | 36.9 | 5.2 | 38.3 | 3 | B |
| ILE | O | 139 | 36.7 | 4.1 | 38.8 | 4 | B |
| ALA | N | 140 | 36.9 | 5.3 | 37.0 | 5 | B |
| ALA | CA | 140 | 36.6 | 4.2 | 36.1 | 7 | B |
| ALA | CB | 140 | 36.1 | 4.7 | 34.8 | 9 | B |
| ALA | C | 140 | 37.9 | 3.5 | 35.9 | 7 | B |
| ALA | O | 140 | 38.8 | 4.0 | 35.3 | 5 | B |
| ALA | N | 141 | 37.9 | 2.2 | 36.4 | 10 | B |
| ALA | CA | 141 | 39.1 | 1.4 | 36.3 | 7 | B |
| ALA | CB | 141 | 39.1 | 0.3 | 37.3 | 7 | B |
| ALA | C | 141 | 39.0 | 0.8 | 34.9 | 9 | B |
| ALA | O | 141 | 38.2 | −0.1 | 34.6 | 8 | B |
| ILE | N | 142 | 39.8 | 1.3 | 33.9 | 8 | B |
| ILE | CA | 142 | 39.9 | 0.8 | 32.6 | 7 | B |
| ILE | CB | 142 | 40.7 | 1.7 | 31.7 | 4 | B |
| ILE | CG2 | 142 | 40.8 | 1.1 | 30.3 | 6 | B |
| ILE | CG1 | 142 | 39.9 | 3.0 | 31.5 | 6 | B |
| ILE | CD1 | 142 | 40.6 | 4.0 | 30.6 | 4 | B |
| ILE | C | 142 | 40.4 | −0.6 | 32.5 | 10 | B |
| ILE | O | 142 | 41.6 | −0.9 | 32.7 | 14 | B |
| THR | N | 143 | 39.5 | −1.6 | 32.1 | 13 | B |
| THR | CA | 143 | 39.9 | −3.0 | 32.0 | 16 | B |
| THR | CB | 143 | 38.7 | −3.9 | 32.6 | 20 | B |
| THR | OG1 | 143 | 37.5 | −3.4 | 32.0 | 20 | B |
| THR | CG2 | 143 | 38.7 | −3.8 | 34.1 | 22 | B |
| THR | C | 143 | 40.2 | −3.4 | 30.6 | 17 | B |
| THR | O | 143 | 40.9 | −4.3 | 30.3 | 18 | B |
| GLU | N | 144 | 39.6 | −2.7 | 29.6 | 21 | B |
| GLU | CA | 144 | 39.8 | −3.0 | 28.2 | 25 | B |
| GLU | CB | 144 | 38.6 | −3.7 | 27.7 | 31 | B |
| GLU | CG | 144 | 38.7 | −4.3 | 26.3 | 35 | B |
| GLU | CD | 144 | 37.6 | −5.3 | 25.9 | 42 | B |
| GLU | OE1 | 144 | 37.6 | −6.4 | 26.6 | 41 | B |
| GLU | OE2 | 144 | 36.8 | −5.1 | 25.0 | 44 | B |
| GLU | C | 144 | 39.9 | −1.7 | 27.5 | 25 | B |
| GLU | O | 144 | 39.2 | −0.7 | 27.8 | 26 | B |
| SER | N | 145 | 40.9 | −1.6 | 26.6 | 26 | B |
| SER | CA | 145 | 41.2 | −0.4 | 25.9 | 28 | B |
| SER | CB | 145 | 42.4 | 0.3 | 26.6 | 31 | B |
| SER | OG | 145 | 43.3 | −0.6 | 27.2 | 33 | B |
| SER | C | 145 | 41.5 | −0.6 | 24.4 | 27 | B |
| SER | O | 145 | 42.1 | −1.7 | 24.0 | 28 | B |
| ASP | N | 146 | 41.2 | 0.3 | 23.6 | 23 | B |
| ASP | CA | 146 | 41.5 | 0.2 | 22.1 | 21 | B |
| ASP | CB | 146 | 40.4 | −0.6 | 21.5 | 22 | B |
| ASP | CG | 146 | 40.5 | −0.7 | 20.0 | 19 | B |
| ASP | OD1 | 146 | 41.6 | −1.0 | 19.5 | 19 | B |
| ASP | OD2 | 146 | 39.5 | −0.4 | 19.3 | 19 | B |
| ASP | C | 146 | 41.5 | 1.6 | 21.4 | 19 | B |
| ASP | O | 146 | 40.6 | 2.3 | 21.4 | 17 | B |
| LYS | N | 147 | 42.7 | 1.9 | 20.9 | 15 | B |
| LYS | CA | 147 | 42.9 | 3.2 | 20.2 | 12 | B |
| LYS | CB | 147 | 41.8 | 3.4 | 19.1 | 14 | B |
| LYS | CG | 147 | 41.8 | 2.4 | 18.1 | 19 | B |
| LYS | CD | 147 | 42.8 | 2.6 | 17.0 | 19 | B |
| LYS | CE | 147 | 42.6 | 1.7 | 15.8 | 19 | B |
| LYS | NZ | 147 | 43.7 | 1.8 | 14.8 | 18 | B |
| LYS | C | 147 | 42.9 | 4.4 | 21.1 | 14 | B |
| LYS | O | 147 | 42.7 | 5.5 | 20.7 | 12 | B |
| PHE | N | 148 | 43.1 | 4.1 | 22.4 | 15 | B |
| PHE | CA | 148 | 43.1 | 5.1 | 23.5 | 15 | B |
| PHE | CB | 148 | 42.3 | 4.7 | 24.7 | 11 | B |
| PHE | CG | 148 | 42.0 | 5.8 | 25.7 | 5 | B |
| PHE | CD1 | 148 | 41.1 | 6.7 | 25.4 | 3 | B |
| PHE | CD2 | 148 | 42.7 | 5.8 | 26.9 | 2 | B |
| PHE | CE1 | 148 | 40.8 | 7.7 | 26.4 | 2 | B |
| PHE | CE2 | 148 | 42.4 | 6.7 | 27.9 | 1 | B |
| PHE | CZ | 148 | 41.4 | 7.7 | 27.6 | 1 | B |
| PHE | C | 148 | 44.6 | 5.4 | 23.9 | 15 | B |
| PHE | O | 148 | 45.2 | 6.4 | 23.5 | 15 | B |
| PHE | N | 149 | 45.1 | 4.6 | 24.8 | 15 | B |
| PHE | CA | 149 | 46.5 | 4.7 | 25.2 | 14 | B |
| PHE | CB | 149 | 46.8 | 3.6 | 26.2 | 13 | B |
| PHE | CG | 149 | 45.9 | 3.5 | 27.4 | 11 | B |
| PHE | CD1 | 149 | 45.4 | 2.3 | 27.9 | 8 | B |
| PHE | CD2 | 149 | 45.7 | 4.7 | 28.1 | 11 | B |
| PHE | CE1 | 149 | 44.7 | 2.2 | 29.0 | 10 | B |
| PHE | CE2 | 149 | 44.9 | 4.6 | 29.3 | 11 | B |
| PHE | CZ | 149 | 44.4 | 3.4 | 29.7 | 14 | B |
| PHE | C | 149 | 47.5 | 4.7 | 24.1 | 15 | B |
| PHE | O | 149 | 47.5 | 3.8 | 23.3 | 17 | B |
| ILE | N | 150 | 48.3 | 5.8 | 24.0 | 15 | B |
| ILE | CA | 150 | 49.2 | 5.9 | 22.9 | 15 | B |
| ILE | CB | 150 | 49.4 | 7.4 | 22.5 | 12 | B |
| ILE | CG2 | 150 | 50.3 | 7.5 | 21.4 | 13 | B |
| ILE | CG1 | 150 | 48.0 | 8.0 | 22.2 | 11 | B |
| ILE | CD1 | 150 | 48.0 | 9.4 | 21.9 | 8 | B |
| ILE | C | 150 | 50.6 | 5.4 | 23.3 | 19 | B |
| ILE | O | 150 | 51.0 | 5.5 | 24.4 | 20 | B |
| ASN | N | 151 | 51.3 | 4.8 | 22.3 | 23 | B |
| ASN | CA | 151 | 52.7 | 4.2 | 22.6 | 24 | B |
| ASN | CB | 151 | 53.2 | 3.5 | 21.5 | 25 | B |
| ASN | CG | 151 | 54.4 | 2.6 | 21.9 | 31 | B |
| ASN | OD1 | 151 | 55.0 | 1.8 | 21.0 | 35 | B |
| ASN | ND2 | 151 | 54.8 | 2.7 | 23.1 | 31 | B |
| ASN | C | 151 | 53.6 | 5.3 | 23.1 | 24 | B |
| ASN | O | 151 | 54.0 | 6.2 | 22.3 | 19 | B |
| GLY | N | 152 | 54.0 | 5.2 | 24.3 | 24 | B |
| GLY | CA | 152 | 55.0 | 6.2 | 24.9 | 26 | B |
| GLY | C | 152 | 54.4 | 7.6 | 25.1 | 25 | B |
| GLY | O | 152 | 55.2 | 8.6 | 25.0 | 25 | B |
| SER | N | 153 | 53.1 | 7.7 | 25.3 | 23 | B |
| SER | CA | 153 | 52.5 | 9.0 | 25.5 | 21 | B |
| SER | CB | 153 | 50.9 | 8.9 | 25.5 | 25 | B |
| SER | OG | 153 | 50.5 | 8.1 | 26.6 | 30 | B |
| SER | C | 153 | 52.9 | 9.5 | 26.9 | 19 | B |
| SER | O | 153 | 53.0 | 10.7 | 27.1 | 17 | B |
| ASN | N | 154 | 53.1 | 8.6 | 27.8 | 17 | B |
| ASN | CA | 154 | 53.5 | 8.8 | 29.1 | 16 | B |
| ASN | CB | 154 | 54.6 | 9.9 | 29.2 | 19 | B |
| ASN | CG | 154 | 55.5 | 9.7 | 30.5 | 24 | B |
| ASN | OD1 | 154 | 55.9 | 8.6 | 30.8 | 24 | B |
| ASN | ND2 | 154 | 55.7 | 10.9 | 31.2 | 26 | B |
| ASN | C | 154 | 52.4 | 9.2 | 30.1 | 11 | B |
| ASN | O | 154 | 52.6 | 9.6 | 31.2 | 11 | B |
| TRP | N | 155 | 51.2 | 9.0 | 29.6 | 9 | B |
| TRP | CA | 155 | 50.0 | 9.2 | 30.5 | 7 | B |
| TRP | CB | 155 | 49.1 | 10.4 | 30.1 | 6 | B |
| TRP | CG | 155 | 48.7 | 10.4 | 28.6 | 5 | B |
| TRP | CD2 | 155 | 47.5 | 9.8 | 28.1 | 4 | B |
| TRP | CE2 | 155 | 47.5 | 10.1 | 26.7 | 2 | B |
| TRP | CE3 | 155 | 46.6 | 8.9 | 28.6 | 8 | B |
| TRP | CD1 | 155 | 49.3 | 11.1 | 27.6 | 6 | B |
| TRP | NE1 | 155 | 48.5 | 11.0 | 26.5 | 2 | B |
| TRP | CZ2 | 155 | 46.5 | 9.7 | 25.9 | 2 | B |
| TRP | CZ3 | 155 | 45.6 | 8.5 | 27.8 | 8 | B |
| TRP | CH2 | 155 | 45.5 | 8.9 | 26.4 | 8 | B |
| TRP | C | 155 | 49.3 | 7.9 | 30.6 | 7 | B |
| TRP | O | 155 | 49.2 | 7.2 | 29.6 | 11 | B |
| GLU | N | 156 | 48.8 | 7.6 | 31.8 | 6 | B |
| GLU | CA | 156 | 48.0 | 6.3 | 31.9 | 6 | B |
| GLU | CB | 156 | 48.7 | 5.5 | 33.0 | 10 | B |
| GLU | CG | 156 | 50.2 | 5.3 | 32.8 | 20 | B |
| GLU | CD | 156 | 51.0 | 6.4 | 33.4 | 26 | B |
| GLU | OE1 | 156 | 52.3 | 6.4 | 33.2 | 28 | B |
| GLU | OE2 | 156 | 50.5 | 7.3 | 34.0 | 29 | B |
| GLU | C | 156 | 46.6 | 6.5 | 32.3 | 6 | B |
| GLU | O | 156 | 45.9 | 5.6 | 32.8 | 10 | B |
| GLY | N | 157 | 46.1 | 7.7 | 32.1 | 3 | B |
| GLY | CA | 157 | 44.7 | 8.0 | 32.5 | 1 | B |
| GLY | C | 157 | 44.2 | 9.3 | 31.9 | 3 | B |
| GLY | O | 157 | 44.9 | 10.0 | 31.2 | 1 | B |
| ILE | N | 158 | 42.9 | 9.5 | 32.1 | 5 | B |
| ILE | CA | 158 | 42.3 | 10.8 | 31.6 | 4 | B |
| ILE | CB | 158 | 41.4 | 10.4 | 30.3 | 1 | B |
| ILE | CG2 | 158 | 40.4 | 9.5 | 30.7 | 2 | B |
| ILE | CG1 | 158 | 40.8 | 11.7 | 29.8 | 5 | B |
| ILE | CD1 | 158 | 39.9 | 11.4 | 28.6 | 7 | B |
| ILE | C | 158 | 41.4 | 11.4 | 32.6 | 4 | B |
| ILE | O | 158 | 40.7 | 10.8 | 33.4 | 4 | B |
| LEU | N | 159 | 41.4 | 12.8 | 32.7 | 5 | B |
| LEU | CA | 159 | 40.7 | 13.6 | 33.6 | 4 | B |
| LEU | CB | 159 | 41.6 | 14.4 | 34.5 | 4 | B |
| LEU | CG | 159 | 41.0 | 15.4 | 35.6 | 1 | B |
| LEU | CD1 | 159 | 40.4 | 14.6 | 36.7 | 1 | B |
| LEU | CD2 | 159 | 42.0 | 16.3 | 36.1 | 1 | B |
| LEU | C | 159 | 39.7 | 14.5 | 32.9 | 3 | B |
| LEU | O | 159 | 40.1 | 15.6 | 32.5 | 1 | B |
| GLY | N | 160 | 38.4 | 14.1 | 32.8 | 3 | B |
| GLY | CA | 160 | 37.5 | 14.9 | 32.1 | 4 | B |
| GLY | C | 160 | 37.0 | 16.0 | 33.0 | 3 | B |
| GLY | O | 160 | 36.3 | 15.8 | 34.0 | 7 | B |
| LEU | N | 161 | 37.4 | 17.3 | 32.6 | 1 | B |
| LEU | CA | 161 | 37.0 | 18.4 | 33.4 | 1 | B |
| LEU | CB | 161 | 38.2 | 19.4 | 33.3 | 1 | B |
| LEU | CG | 161 | 39.5 | 18.9 | 34.0 | 1 | B |
| LEU | CD1 | 161 | 40.6 | 19.9 | 33.7 | 1 | B |
| LEU | CD2 | 161 | 39.2 | 18.9 | 35.5 | 1 | B |
| LEU | C | 161 | 35.7 | 19.1 | 32.9 | 1 | B |
| LEU | O | 161 | 35.4 | 20.1 | 33.4 | 1 | B |
| ALA | N | 162 | 35.1 | 18.4 | 32.0 | 3 | B |
| ALA | CA | 162 | 33.8 | 19.0 | 31.5 | 1 | B |
| ALA | CB | 162 | 33.4 | 18.2 | 30.2 | 3 | B |
| ALA | C | 162 | 32.7 | 18.8 | 32.5 | 1 | B |
| ALA | O | 162 | 33.0 | 18.6 | 33.7 | 1 | B |
| TYR | N | 163 | 31.5 | 19.0 | 32.1 | 1 | B |
| TYR | CA | 163 | 30.4 | 18.9 | 33.1 | 2 | B |
| TYR | CB | 163 | 29.3 | 19.9 | 32.7 | 4 | B |
| TYR | CG | 163 | 29.8 | 21.3 | 32.9 | 1 | B |
| TYR | CD1 | 163 | 30.6 | 21.9 | 31.9 | 1 | B |
| TYR | CE1 | 163 | 31.0 | 23.3 | 32.0 | 1 | B |
| TYR | CD2 | 163 | 29.4 | 22.1 | 33.9 | 1 | B |
| TYR | CE2 | 163 | 29.8 | 23.4 | 34.1 | 3 | B |
| TYR | CZ | 163 | 30.6 | 24.0 | 33.1 | 2 | B |
| TYR | OH | 163 | 30.9 | 25.3 | 33.3 | 4 | B |
| TYR | C | 163 | 29.7 | 17.5 | 33.0 | 3 | B |
| TYR | O | 163 | 29.9 | 16.7 | 32.1 | 4 | B |
| ALA | N | 164 | 29.0 | 17.1 | 34.1 | 6 | B |
| ALA | CA | 164 | 28.3 | 15.8 | 34.2 | 5 | B |
| ALA | CB | 164 | 27.6 | 15.7 | 35.5 | 8 | B |
| ALA | C | 164 | 27.4 | 15.5 | 33.1 | 3 | B |
| ALA | O | 164 | 27.2 | 14.3 | 32.8 | 4 | B |
| GLU | N | 165 | 26.7 | 16.5 | 32.5 | 5 | B |
| GLU | CA | 165 | 25.7 | 16.3 | 31.5 | 6 | B |
| GLU | CB | 165 | 25.4 | 17.6 | 30.9 | 9 | B |
| GLU | CG | 165 | 24.5 | 17.5 | 29.6 | 15 | B |
| GLU | CD | 165 | 23.0 | 17.6 | 29.9 | 20 | B |
| GLU | OE1 | 165 | 22.6 | 18.7 | 30.3 | 28 | B |
| GLU | OE2 | 165 | 22.3 | 16.6 | 29.7 | 17 | B |
| GLU | C | 165 | 26.2 | 15.3 | 30.4 | 4 | B |
| GLU | O | 165 | 25.4 | 14.6 | 29.8 | 3 | B |
| ILE | N | 166 | 27.5 | 15.2 | 30.1 | 1 | B |
| ILE | CA | 166 | 28.0 | 14.3 | 29.1 | 1 | B |
| ILE | CB | 166 | 28.9 | 15.0 | 28.1 | 1 | B |
| ILE | CG2 | 166 | 28.1 | 16.2 | 27.4 | 2 | B |
| ILE | CG1 | 166 | 30.2 | 15.5 | 28.8 | 1 | B |
| ILE | CD1 | 166 | 31.2 | 16.1 | 27.8 | 1 | B |
| ILE | C | 166 | 28.8 | 13.2 | 29.7 | 3 | B |
| ILE | O | 166 | 29.5 | 12.5 | 28.9 | 5 | B |
| ALA | N | 167 | 28.7 | 13.0 | 31.0 | 3 | B |
| ALA | CA | 167 | 29.5 | 11.9 | 31.6 | 5 | B |
| ALA | CB | 167 | 29.7 | 12.2 | 33.0 | 7 | B |
| ALA | C | 167 | 28.8 | 10.6 | 31.4 | 6 | B |
| ALA | O | 167 | 27.6 | 10.5 | 31.6 | 7 | B |
| ARG | N | 168 | 29.6 | 9.5 | 31.1 | 5 | B |
| ARG | CA | 168 | 29.0 | 8.2 | 30.9 | 3 | B |
| ARG | CB | 168 | 29.9 | 7.4 | 30.0 | 7 | B |
| ARG | CG | 168 | 30.0 | 8.0 | 28.6 | 11 | B |
| ARG | CD | 168 | 28.8 | 7.4 | 27.7 | 15 | B |
| ARG | NE | 168 | 28.9 | 7.9 | 26.3 | 17 | B |
| ARG | CZ | 168 | 28.3 | 7.2 | 25.3 | 23 | B |
| ARG | NH1 | 168 | 27.7 | 6.1 | 25.5 | 22 | B |
| ARG | NH2 | 168 | 28.4 | 7.7 | 24.1 | 22 | B |
| ARG | C | 168 | 29.1 | 7.5 | 32.3 | 2 | B |
| ARG | O | 168 | 30.0 | 7.8 | 33.1 | 1 | B |
| PRO | N | 169 | 28.1 | 6.6 | 32.7 | 1 | B |
| PRO | CD | 169 | 28.2 | 6.0 | 34.0 | 6 | B |
| PRO | CA | 169 | 27.0 | 6.1 | 31.9 | 1 | B |
| PRO | CB | 169 | 26.5 | 5.0 | 32.8 | 3 | B |
| PRO | CG | 169 | 27.7 | 4.6 | 33.7 | 6 | B |
| PRO | C | 169 | 25.9 | 7.2 | 31.8 | 3 | B |
| PRO | O | 169 | 25.3 | 7.3 | 30.8 | 7 | B |
| ASP | N | 170 | 25.7 | 7.9 | 32.9 | 4 | B |
| ASP | CA | 170 | 24.7 | 9.0 | 32.9 | 5 | B |
| ASP | CB | 170 | 23.3 | 8.5 | 33.3 | 6 | B |
| ASP | CG | 170 | 23.3 | 8.0 | 34.8 | 8 | B |
| ASP | OD1 | 170 | 23.6 | 6.8 | 35.0 | 9 | B |
| ASP | OD2 | 170 | 23.1 | 8.8 | 35.7 | 14 | B |
| ASP | C | 170 | 25.1 | 10.2 | 33.7 | 5 | B |
| ASP | O | 170 | 26.1 | 10.1 | 34.4 | 1 | B |
| ASP | N | 171 | 24.4 | 11.3 | 33.6 | 8 | B |
| ASP | CA | 171 | 24.7 | 12.5 | 34.3 | 11 | B |
| ASP | CB | 171 | 23.8 | 13.6 | 33.9 | 13 | B |
| ASP | CG | 171 | 22.3 | 13.3 | 34.2 | 15 | B |
| ASP | OD1 | 171 | 22.0 | 12.3 | 34.8 | 14 | B |
| ASP | OD2 | 171 | 21.5 | 14.2 | 33.9 | 17 | B |
| ASP | C | 171 | 24.6 | 12.4 | 35.8 | 12 | B |
| ASP | O | 171 | 24.5 | 13.4 | 36.5 | 13 | B |
| SER | N | 172 | 24.8 | 11.2 | 36.4 | 11 | B |
| SER | CA | 172 | 24.8 | 11.1 | 37.8 | 8 | B |
| SER | CB | 172 | 23.8 | 9.9 | 38.2 | 6 | B |
| SER | OG | 172 | 24.3 | 8.7 | 38.0 | 7 | B |
| SER | C | 172 | 26.2 | 10.7 | 38.3 | 7 | B |
| SER | O | 172 | 26.4 | 10.7 | 39.6 | 11 | B |
| LEU | N | 173 | 27.1 | 10.4 | 37.4 | 4 | B |
| LEU | CA | 173 | 28.4 | 10.1 | 37.9 | 2 | B |
| LEU | CB | 173 | 29.1 | 9.2 | 36.8 | 1 | B |
| LEU | CG | 173 | 30.3 | 8.4 | 37.2 | 1 | B |
| LEU | CD1 | 173 | 30.1 | 7.6 | 38.4 | 1 | B |
| LEU | CD2 | 173 | 30.8 | 7.5 | 36.0 | 1 | B |
| LEU | C | 173 | 29.2 | 11.4 | 38.0 | 4 | B |
| LEU | O | 173 | 30.0 | 11.8 | 37.1 | 2 | B |
| GLU | N | 174 | 29.0 | 12.0 | 39.2 | 5 | B |
| GLU | CA | 174 | 29.7 | 13.3 | 39.5 | 6 | B |
| GLU | CB | 174 | 29.5 | 13.6 | 41.0 | 8 | B |
| GLU | CG | 174 | 30.2 | 14.8 | 41.5 | 10 | B |
| GLU | CD | 174 | 29.9 | 15.1 | 42.9 | 10 | B |
| GLU | OE1 | 174 | 28.7 | 15.6 | 43.2 | 15 | B |
| GLU | OE2 | 174 | 30.7 | 14.9 | 43.8 | 10 | B |
| GLU | C | 174 | 31.2 | 13.3 | 39.2 | 5 | B |
| GLU | O | 174 | 32.0 | 12.6 | 39.8 | 8 | B |
| PRO | N | 175 | 31.5 | 14.2 | 38.2 | 3 | B |
| PRO | CD | 175 | 30.5 | 14.9 | 37.4 | 3 | B |
| PRO | CA | 175 | 32.9 | 14.4 | 37.6 | 4 | B |
| PRO | CB | 175 | 32.6 | 15.5 | 36.6 | 3 | B |
| PRO | CG | 175 | 31.2 | 15.2 | 36.2 | 3 | B |
| PRO | C | 175 | 33.9 | 14.9 | 38.7 | 2 | B |
| PRO | O | 175 | 33.6 | 15.7 | 39.6 | 4 | B |
| PHE | N | 176 | 35.2 | 14.5 | 38.5 | 3 | B |
| PHE | CA | 176 | 36.2 | 14.8 | 39.5 | 1 | B |
| PHE | CB | 176 | 37.6 | 14.7 | 38.8 | 1 | B |
| PHE | CG | 176 | 38.7 | 15.1 | 39.8 | 1 | B |
| PHE | CD1 | 176 | 39.2 | 14.2 | 40.7 | 5 | B |
| PHE | CD2 | 176 | 39.2 | 16.3 | 39.7 | 1 | B |
| PHE | CE1 | 176 | 40.2 | 14.5 | 41.6 | 2 | B |
| PHE | CE2 | 176 | 40.2 | 16.7 | 40.6 | 2 | B |
| PHE | CZ | 176 | 40.7 | 15.8 | 41.5 | 2 | B |
| PHE | C | 176 | 36.1 | 16.2 | 40.0 | 1 | B |
| PHE | O | 176 | 36.0 | 16.4 | 41.2 | 4 | B |
| PHE | N | 177 | 36.2 | 17.2 | 39.1 | 2 | B |
| PHE | CA | 177 | 36.2 | 18.6 | 39.6 | 3 | B |
| PHE | CB | 177 | 36.2 | 19.6 | 38.4 | 1 | B |
| PHE | CG | 177 | 36.8 | 20.9 | 38.7 | 5 | B |
| PHE | CD1 | 177 | 38.2 | 20.9 | 38.8 | 5 | B |
| PHE | CD2 | 177 | 36.1 | 22.0 | 38.8 | 3 | B |
| PHE | CE1 | 177 | 38.9 | 22.1 | 39.1 | 6 | B |
| PHE | CE2 | 177 | 36.8 | 23.2 | 39.1 | 2 | B |
| PHE | CZ | 177 | 38.1 | 23.3 | 39.2 | 4 | B |
| PHE | C | 177 | 35.0 | 18.9 | 40.5 | 5 | B |
| PHE | O | 177 | 35.1 | 19.4 | 41.6 | 1 | B |
| ASP | N | 178 | 33.8 | 18.5 | 40.0 | 7 | B |
| ASP | CA | 178 | 32.5 | 18.7 | 40.7 | 10 | B |
| ASP | CB | 178 | 31.4 | 18.0 | 40.0 | 11 | B |
| ASP | CG | 178 | 30.6 | 18.9 | 39.1 | 16 | B |
| ASP | OD1 | 178 | 30.2 | 20.0 | 39.6 | 19 | B |
| ASP | OD2 | 178 | 30.4 | 18.6 | 37.9 | 16 | B |
| ASP | C | 178 | 32.7 | 18.1 | 42.1 | 11 | B |
| ASP | O | 178 | 32.3 | 18.8 | 43.1 | 13 | B |
| SER | N | 179 | 33.3 | 17.0 | 42.3 | 9 | B |
| SER | CA | 179 | 33.5 | 16.4 | 43.6 | 7 | B |
| SER | CB | 179 | 34.1 | 15.0 | 43.5 | 4 | B |
| SER | OG | 179 | 33.1 | 14.1 | 42.9 | 7 | B |
| SER | C | 179 | 34.5 | 17.3 | 44.3 | 5 | B |
| SER | O | 179 | 34.3 | 17.7 | 45.4 | 10 | B |
| LEU | N | 180 | 35.6 | 17.6 | 43.7 | 3 | B |
| LEU | CA | 180 | 36.6 | 18.5 | 44.3 | 1 | B |
| LEU | CB | 180 | 37.7 | 18.9 | 43.4 | 1 | B |
| LEU | CG | 180 | 38.8 | 19.7 | 44.1 | 1 | B |
| LEU | CD1 | 180 | 39.6 | 18.8 | 45.0 | 1 | B |
| LEU | CD2 | 180 | 39.7 | 20.4 | 43.1 | 1 | B |
| LEU | C | 180 | 36.0 | 19.8 | 44.9 | 2 | B |
| LEU | O | 180 | 36.4 | 20.1 | 46.1 | 2 | B |
| VAL | N | 181 | 35.2 | 20.4 | 44.2 | 3 | B |
| VAL | CA | 181 | 34.6 | 21.7 | 44.7 | 4 | B |
| VAL | CB | 181 | 33.9 | 22.5 | 43.6 | 2 | B |
| VAL | CG1 | 181 | 33.1 | 23.6 | 44.3 | 7 | B |
| VAL | CG2 | 181 | 34.9 | 23.1 | 42.7 | 5 | B |
| VAL | C | 181 | 33.7 | 21.5 | 45.9 | 4 | B |
| VAL | O | 181 | 33.7 | 22.2 | 46.9 | 5 | B |
| LYS | N | 182 | 32.8 | 20.5 | 45.8 | 5 | B |
| LYS | CA | 182 | 31.9 | 20.2 | 46.8 | 3 | B |
| LYS | CB | 182 | 30.8 | 19.2 | 46.4 | 1 | B |
| LYS | CG | 182 | 30.2 | 19.6 | 45.1 | 1 | B |
| LYS | CD | 182 | 29.2 | 18.5 | 44.6 | 3 | B |
| LYS | CE | 182 | 27.8 | 18.6 | 45.3 | 9 | B |
| LYS | NZ | 182 | 27.0 | 19.8 | 44.8 | 12 | B |
| LYS | C | 182 | 32.5 | 19.7 | 48.1 | 2 | B |
| LYS | O | 182 | 32.0 | 19.9 | 49.2 | 1 | B |
| GLN | N | 183 | 33.7 | 19.1 | 48.0 | 1 | B |
| GLN | CA | 183 | 34.4 | 18.6 | 49.1 | 3 | B |
| GLN | CB | 183 | 35.0 | 17.2 | 48.8 | 3 | B |
| GLN | CG | 183 | 34.0 | 16.1 | 48.7 | 9 | B |
| GLN | CD | 183 | 34.6 | 14.9 | 48.0 | 9 | B |
| GLN | OE1 | 183 | 35.7 | 14.4 | 48.3 | 10 | B |
| GLN | NE2 | 183 | 33.9 | 14.4 | 47.0 | 10 | B |
| GLN | C | 183 | 35.5 | 19.4 | 49.8 | 5 | B |
| GLN | O | 183 | 36.1 | 19.1 | 50.8 | 4 | B |
| THR | N | 184 | 35.9 | 20.5 | 49.1 | 7 | B |
| THR | CA | 184 | 36.9 | 21.4 | 49.6 | 8 | B |
| THR | CB | 184 | 38.3 | 21.2 | 49.0 | 8 | B |
| THR | OG1 | 184 | 38.2 | 21.5 | 47.6 | 5 | B |
| THR | CG2 | 184 | 38.7 | 19.7 | 49.1 | 7 | B |
| THR | C | 184 | 36.5 | 22.9 | 49.4 | 11 | B |
| THR | O | 184 | 35.4 | 23.2 | 48.9 | 13 | B |
| HIS | N | 185 | 37.4 | 23.8 | 49.8 | 10 | B |
| HIS | CA | 185 | 37.1 | 25.2 | 49.6 | 13 | B |
| HIS | CB | 185 | 37.6 | 26.0 | 50.8 | 16 | B |
| HIS | CG | 185 | 36.8 | 25.8 | 52.0 | 21 | B |
| HIS | CD2 | 185 | 35.8 | 26.5 | 52.5 | 23 | B |
| HIS | ND1 | 185 | 36.9 | 24.7 | 52.8 | 26 | B |
| HIS | CE1 | 185 | 36.0 | 24.8 | 53.8 | 25 | B |
| HIS | NE2 | 185 | 35.3 | 25.9 | 53.6 | 27 | B |
| HIS | C | 185 | 37.8 | 25.8 | 48.3 | 10 | B |
| HIS | O | 185 | 37.8 | 27.0 | 48.0 | 12 | B |
| VAL | N | 186 | 38.3 | 24.8 | 47.5 | 8 | B |
| VAL | CA | 186 | 38.9 | 25.2 | 46.2 | 6 | B |
| VAL | CB | 186 | 39.4 | 24.0 | 45.4 | 7 | B |
| VAL | CG1 | 186 | 40.0 | 24.4 | 44.1 | 5 | B |
| VAL | CG2 | 186 | 40.5 | 23.2 | 46.2 | 7 | B |
| VAL | C | 186 | 37.9 | 25.9 | 45.4 | 3 | B |
| VAL | O | 186 | 36.8 | 25.4 | 45.1 | 3 | B |
| PRO | N | 187 | 38.2 | 27.2 | 45.0 | 1 | B |
| PRO | CD | 187 | 39.3 | 28.0 | 45.4 | 2 | B |
| PRO | CA | 187 | 37.3 | 28.0 | 44.2 | 1 | B |
| PRO | CB | 187 | 38.1 | 29.1 | 43.7 | 3 | B |
| PRO | CG | 187 | 38.9 | 29.4 | 45.0 | 6 | B |
| PRO | C | 187 | 36.8 | 27.1 | 43.0 | 1 | B |
| PRO | O | 187 | 37.4 | 26.2 | 42.6 | 1 | B |
| ASN | N | 188 | 35.6 | 27.4 | 42.5 | 1 | B |
| ASN | CA | 188 | 35.0 | 26.7 | 41.4 | 1 | B |
| ASN | CB | 188 | 33.5 | 26.7 | 41.4 | 1 | B |
| ASN | CG | 188 | 32.9 | 25.8 | 40.4 | 1 | B |
| ASN | OD1 | 188 | 33.5 | 24.9 | 39.8 | 1 | B |
| ASN | ND2 | 188 | 31.6 | 26.0 | 40.1 | 1 | B |
| ASN | C | 188 | 35.5 | 27.1 | 40.0 | 1 | B |
| ASN | O | 188 | 34.7 | 27.6 | 39.2 | 1 | B |
| LEU | N | 189 | 36.8 | 26.9 | 39.7 | 1 | B |
| LEU | CA | 189 | 37.3 | 27.3 | 38.4 | 1 | B |
| LEU | CB | 189 | 37.1 | 28.8 | 38.2 | 1 | B |
| LEU | CG | 189 | 38.0 | 29.7 | 39.0 | 1 | B |
| LEU | CD1 | 189 | 39.2 | 30.2 | 38.1 | 2 | B |
| LEU | CD2 | 189 | 37.3 | 30.9 | 39.6 | 2 | B |
| LEU | C | 189 | 38.8 | 26.9 | 38.3 | 1 | B |
| LEU | O | 189 | 39.5 | 26.9 | 39.2 | 3 | B |
| PHE | N | 190 | 39.2 | 26.7 | 37.0 | 1 | B |
| PHE | CA | 190 | 40.6 | 26.3 | 36.8 | 1 | B |
| PHE | CB | 190 | 40.8 | 24.8 | 36.6 | 1 | B |
| PHE | CG | 190 | 40.1 | 24.2 | 35.4 | 1 | B |
| PHE | CD1 | 190 | 38.8 | 23.8 | 35.4 | 1 | B |
| PHE | CD2 | 190 | 40.8 | 24.1 | 34.2 | 1 | B |
| PHE | CE1 | 190 | 38.1 | 23.3 | 34.3 | 1 | B |
| PHE | CE2 | 190 | 40.2 | 23.6 | 33.1 | 1 | B |
| PHE | CZ | 190 | 38.8 | 23.2 | 33.1 | 1 | B |
| PHE | C | 190 | 41.0 | 27.0 | 35.5 | 1 | B |
| PHE | O | 190 | 40.2 | 27.3 | 34.6 | 2 | B |
| SER | N | 191 | 42.3 | 27.2 | 35.3 | 2 | B |
| SER | CA | 191 | 42.9 | 27.9 | 34.2 | 2 | B |
| SER | CB | 191 | 43.4 | 29.3 | 34.5 | 3 | B |
| SER | OG | 191 | 44.5 | 29.2 | 35.3 | 9 | B |
| SER | C | 191 | 44.0 | 27.0 | 33.6 | 1 | B |
| SER | O | 191 | 44.8 | 26.4 | 34.4 | 1 | B |
| LEU | N | 192 | 44.1 | 26.9 | 32.3 | 1 | B |
| LEU | CA | 192 | 45.2 | 26.2 | 31.6 | 1 | B |
| LEU | CB | 192 | 44.5 | 25.0 | 30.8 | 1 | B |
| LEU | CG | 192 | 44.0 | 23.8 | 31.4 | 1 | B |
| LEU | CD1 | 192 | 43.1 | 23.0 | 30.4 | 1 | B |
| LEU | CD2 | 192 | 45.1 | 22.9 | 32.0 | 1 | B |
| LEU | C | 192 | 46.0 | 27.0 | 30.7 | 1 | B |
| LEU | O | 192 | 45.5 | 27.9 | 30.0 | 1 | B |
| GLN | N | 193 | 47.3 | 26.8 | 30.8 | 1 | B |
| GLN | CA | 193 | 48.2 | 27.5 | 29.9 | 1 | B |
| GLN | CB | 193 | 49.2 | 28.4 | 30.7 | 1 | B |
| GLN | CG | 193 | 50.1 | 29.1 | 29.8 | 2 | B |
| GLN | CD | 193 | 51.5 | 29.4 | 30.4 | 4 | B |
| GLN | OE1 | 193 | 52.2 | 28.5 | 30.8 | 3 | B |
| GLN | NE2 | 193 | 51.9 | 30.7 | 30.5 | 6 | B |
| GLN | C | 193 | 49.0 | 26.4 | 29.2 | 1 | B |
| GLN | O | 193 | 50.0 | 26.0 | 29.7 | 2 | B |
| LEU | N | 194 | 48.5 | 26.0 | 28.0 | 1 | B |
| LEU | CA | 194 | 49.2 | 25.0 | 27.3 | 1 | B |
| LEU | CB | 194 | 48.2 | 24.2 | 26.5 | 1 | B |
| LEU | CG | 194 | 47.1 | 23.5 | 27.3 | 1 | B |
| LEU | CD1 | 194 | 46.0 | 22.9 | 26.5 | 1 | B |
| LEU | CD2 | 194 | 47.7 | 22.6 | 28.3 | 1 | B |
| LEU | C | 194 | 50.2 | 25.6 | 26.4 | 3 | B |
| LEU | O | 194 | 49.9 | 26.6 | 25.7 | 4 | B |
| CYS | N | 195 | 51.5 | 25.2 | 26.5 | 1 | B |
| CYS | CA | 195 | 52.5 | 25.8 | 25.7 | 3 | B |
| CYS | C | 195 | 53.0 | 24.7 | 24.7 | 7 | B |
| CYS | O | 195 | 53.4 | 23.6 | 25.1 | 8 | B |
| CYS | CB | 195 | 53.7 | 26.2 | 26.5 | 5 | B |
| CYS | SG | 195 | 53.2 | 27.2 | 28.0 | 3 | B |
| GLY | N | 196 | 52.9 | 25.0 | 23.4 | 11 | B |
| GLY | CA | 196 | 53.4 | 24.1 | 22.4 | 12 | B |
| GLY | C | 196 | 54.9 | 24.1 | 22.4 | 14 | B |
| GLY | O | 196 | 55.5 | 24.8 | 23.2 | 10 | B |
| ALA | N | 197 | 55.5 | 23.3 | 21.5 | 16 | B |
| ALA | CA | 197 | 56.9 | 23.2 | 21.5 | 20 | B |
| ALA | CB | 197 | 57.3 | 21.9 | 20.9 | 16 | B |
| ALA | C | 197 | 57.6 | 24.4 | 20.7 | 23 | B |
| ALA | O | 197 | 58.2 | 25.3 | 21.3 | 24 | B |
| GLY | N | 198 | 57.4 | 24.3 | 19.4 | 28 | B |
| GLY | CA | 198 | 58.0 | 25.3 | 18.5 | 31 | B |
| GLY | C | 198 | 59.3 | 24.7 | 17.9 | 32 | B |
| GLY | O | 198 | 59.7 | 25.0 | 16.8 | 32 | B |
| PHE | N | 199 | 59.9 | 23.8 | 18.7 | 34 | B |
| PHE | CA | 199 | 61.1 | 23.1 | 18.4 | 38 | B |
| PHE | CB | 199 | 62.3 | 23.5 | 19.3 | 37 | B |
| PHE | CG | 199 | 62.0 | 24.7 | 20.2 | 36 | B |
| PHE | CD1 | 199 | 61.8 | 25.9 | 19.6 | 33 | B |
| PHE | CD2 | 199 | 62.0 | 24.5 | 21.6 | 33 | B |
| PHE | CE1 | 199 | 61.6 | 27.0 | 20.4 | 34 | B |
| PHE | CE2 | 199 | 61.8 | 25.6 | 22.4 | 34 | B |
| PHE | CZ | 199 | 61.6 | 26.9 | 21.8 | 36 | B |
| PHE | C | 199 | 60.7 | 21.6 | 18.6 | 39 | B |
| PHE | O | 199 | 59.8 | 21.2 | 19.3 | 41 | B |
| PRO | N | 200 | 61.4 | 20.7 | 17.9 | 40 | B |
| PRO | CD | 200 | 62.5 | 20.9 | 16.8 | 41 | B |
| PRO | CA | 200 | 61.1 | 19.3 | 18.0 | 40 | B |
| PRO | CB | 200 | 61.9 | 18.6 | 16.9 | 40 | B |
| PRO | CG | 200 | 63.1 | 19.5 | 16.7 | 42 | B |
| PRO | C | 200 | 61.4 | 18.6 | 19.4 | 41 | B |
| PRO | O | 200 | 62.3 | 19.0 | 20.1 | 42 | B |
| LEU | N | 201 | 60.5 | 17.7 | 19.7 | 40 | B |
| LEU | CA | 201 | 60.6 | 16.9 | 21.0 | 34 | B |
| LEU | CB | 201 | 59.8 | 17.6 | 22.1 | 31 | B |
| LEU | CG | 201 | 60.2 | 18.9 | 22.7 | 29 | B |
| LEU | CD1 | 201 | 59.5 | 19.1 | 24.0 | 29 | B |
| LEU | CD2 | 201 | 61.7 | 18.9 | 22.9 | 25 | B |
| LEU | C | 201 | 60.1 | 15.5 | 20.8 | 34 | B |
| LEU | O | 201 | 58.9 | 15.3 | 20.8 | 32 | B |
| ASN | N | 202 | 61.0 | 14.5 | 20.5 | 33 | B |
| ASN | CA | 202 | 60.6 | 13.1 | 20.3 | 35 | B |
| ASN | CB | 202 | 61.8 | 12.3 | 20.0 | 35 | B |
| ASN | CG | 202 | 62.7 | 12.0 | 21.2 | 40 | B |
| ASN | OD1 | 202 | 63.3 | 13.0 | 21.7 | 40 | B |
| ASN | ND2 | 202 | 62.9 | 10.8 | 21.6 | 37 | B |
| ASN | C | 202 | 59.9 | 12.6 | 21.6 | 34 | B |
| ASN | O | 202 | 59.3 | 13.4 | 22.3 | 33 | B |
| GLN | N | 203 | 59.9 | 11.3 | 21.8 | 35 | B |
| GLN | CA | 203 | 59.3 | 10.7 | 23.0 | 36 | B |
| GLN | CB | 203 | 59.2 | 9.2 | 22.8 | 34 | B |
| GLN | CG | 203 | 58.4 | 8.5 | 24.0 | 35 | B |
| GLN | CD | 203 | 59.3 | 8.1 | 25.2 | 40 | B |
| GLN | OE1 | 203 | 59.5 | 8.8 | 26.1 | 39 | B |
| GLN | NE2 | 203 | 59.8 | 6.8 | 25.1 | 38 | B |
| GLN | C | 203 | 60.1 | 11.0 | 24.3 | 36 | B |
| GLN | O | 203 | 59.5 | 11.4 | 25.3 | 38 | B |
| SER | N | 204 | 61.4 | 10.9 | 24.2 | 38 | B |
| SER | CA | 204 | 62.3 | 11.2 | 25.3 | 38 | B |
| SER | CB | 204 | 63.7 | 10.5 | 25.1 | 38 | B |
| SER | OG | 204 | 64.5 | 10.5 | 26.3 | 40 | B |
| SER | C | 204 | 62.5 | 12.7 | 25.6 | 40 | B |
| SER | O | 204 | 63.5 | 13.1 | 26.2 | 39 | B |
| GLU | N | 205 | 61.5 | 13.5 | 25.3 | 41 | B |
| GLU | CA | 205 | 61.6 | 15.0 | 25.5 | 41 | B |
| GLU | CB | 205 | 61.7 | 15.7 | 24.2 | 38 | B |
| GLU | CG | 205 | 63.1 | 15.6 | 23.5 | 42 | B |
| GLU | CD | 205 | 64.2 | 16.3 | 24.3 | 44 | B |
| GLU | OE1 | 205 | 64.5 | 15.9 | 25.4 | 44 | B |
| GLU | OE2 | 205 | 64.8 | 17.3 | 23.7 | 47 | B |
| GLU | C | 205 | 60.3 | 15.5 | 26.3 | 41 | B |
| GLU | O | 205 | 59.9 | 16.6 | 26.2 | 39 | B |
| VAL | N | 206 | 59.8 | 14.6 | 27.2 | 40 | B |
| VAL | CA | 206 | 58.6 | 14.9 | 28.0 | 37 | B |
| VAL | CB | 206 | 57.4 | 14.0 | 27.6 | 33 | B |
| VAL | CG1 | 206 | 56.1 | 14.7 | 27.9 | 32 | B |
| VAL | CG2 | 206 | 57.5 | 13.5 | 26.2 | 33 | B |
| VAL | C | 206 | 58.9 | 14.7 | 29.5 | 38 | B |
| VAL | O | 206 | 58.7 | 15.7 | 30.3 | 37 | B |
| LEU | N | 207 | 59.3 | 13.5 | 29.8 | 39 | B |
| LEU | CA | 207 | 59.6 | 13.1 | 31.2 | 38 | B |
| LEU | CB | 207 | 60.1 | 11.7 | 31.3 | 34 | B |
| LEU | CG | 207 | 59.2 | 10.5 | 30.7 | 31 | B |
| LEU | CD1 | 207 | 59.4 | 10.4 | 29.2 | 28 | B |
| LEU | CD2 | 207 | 59.8 | 9.2 | 31.3 | 27 | B |
| LEU | C | 207 | 60.7 | 14.0 | 31.9 | 38 | B |
| LEU | O | 207 | 60.6 | 14.3 | 33.1 | 36 | B |
| ALA | N | 208 | 61.6 | 14.5 | 31.0 | 40 | B |
| ALA | CA | 208 | 62.7 | 15.3 | 31.5 | 40 | B |
| ALA | CB | 208 | 64.0 | 15.0 | 30.7 | 37 | B |
| ALA | C | 208 | 62.4 | 16.8 | 31.4 | 40 | B |
| ALA | O | 208 | 63.1 | 17.7 | 31.9 | 39 | B |
| SER | N | 209 | 61.2 | 17.1 | 30.9 | 42 | B |
| SER | CA | 209 | 60.7 | 18.5 | 30.7 | 41 | B |
| SER | CB | 209 | 60.9 | 18.9 | 29.3 | 40 | B |
| SER | OG | 209 | 62.3 | 19.2 | 28.9 | 42 | B |
| SER | C | 209 | 59.3 | 18.9 | 31.2 | 40 | B |
| SER | O | 209 | 58.8 | 18.3 | 32.2 | 37 | B |
| VAL | N | 210 | 58.8 | 19.9 | 30.6 | 40 | B |
| VAL | CA | 210 | 57.5 | 20.4 | 30.9 | 36 | B |
| VAL | CB | 210 | 57.5 | 21.5 | 32.1 | 33 | B |
| VAL | CG1 | 210 | 56.2 | 22.1 | 32.4 | 32 | B |
| VAL | CG2 | 210 | 58.1 | 20.8 | 33.3 | 30 | B |
| VAL | C | 210 | 56.8 | 21.1 | 29.7 | 34 | B |
| VAL | O | 210 | 57.5 | 21.9 | 28.9 | 38 | B |
| GLY | N | 211 | 55.5 | 20.9 | 29.5 | 28 | B |
| GLY | CA | 211 | 54.8 | 21.4 | 28.3 | 15 | B |
| GLY | C | 211 | 53.9 | 22.6 | 28.7 | 11 | B |
| GLY | O | 211 | 53.5 | 23.4 | 27.9 | 10 | B |
| GLY | N | 212 | 53.6 | 22.8 | 30.0 | 5 | B |
| GLY | CA | 212 | 52.8 | 23.9 | 30.4 | 4 | B |
| GLY | C | 212 | 52.2 | 23.8 | 31.8 | 2 | B |
| GLY | O | 212 | 52.7 | 23.0 | 32.6 | 5 | B |
| SER | N | 213 | 51.2 | 24.6 | 32.2 | 1 | B |
| SER | CA | 213 | 50.6 | 24.6 | 33.5 | 1 | B |
| SER | CB | 213 | 51.2 | 25.7 | 34.4 | 1 | B |
| SER | OG | 213 | 51.5 | 26.8 | 33.6 | 1 | B |
| SER | C | 213 | 49.1 | 24.6 | 33.6 | 1 | B |
| SER | O | 213 | 48.4 | 25.3 | 32.8 | 1 | B |
| MET | N | 214 | 48.5 | 23.9 | 34.6 | 1 | B |
| MET | CA | 214 | 47.1 | 23.9 | 34.9 | 1 | B |
| MET | CB | 214 | 46.5 | 22.5 | 34.9 | 1 | B |
| MET | CG | 214 | 45.2 | 22.4 | 35.5 | 1 | B |
| MET | SD | 214 | 44.3 | 20.8 | 35.4 | 1 | B |
| MET | CE | 214 | 44.8 | 20.0 | 36.8 | 1 | B |
| MET | C | 214 | 46.9 | 24.4 | 36.3 | 1 | B |
| MET | O | 214 | 47.2 | 23.7 | 37.3 | 1 | B |
| ILE | N | 215 | 46.4 | 25.7 | 36.5 | 2 | B |
| ILE | CA | 215 | 46.2 | 26.3 | 37.8 | 3 | B |
| ILE | CB | 215 | 46.3 | 27.8 | 37.7 | 1 | B |
| ILE | CG2 | 215 | 46.0 | 28.4 | 39.0 | 1 | B |
| ILE | CG1 | 215 | 47.6 | 28.2 | 37.2 | 1 | B |
| ILE | CD1 | 215 | 48.8 | 27.5 | 37.8 | 1 | B |
| ILE | C | 215 | 44.8 | 25.9 | 38.3 | 7 | B |
| ILE | O | 215 | 43.8 | 26.3 | 37.8 | 10 | B |
| ILE | N | 216 | 44.8 | 25.1 | 39.4 | 7 | B |
| ILE | CA | 216 | 43.5 | 24.7 | 40.0 | 3 | B |
| ILE | CB | 216 | 43.6 | 23.4 | 40.8 | 6 | B |
| ILE | CG2 | 216 | 42.3 | 23.0 | 41.4 | 5 | B |
| ILE | CG1 | 216 | 44.2 | 22.3 | 39.9 | 7 | B |
| ILE | CD1 | 216 | 43.3 | 22.0 | 38.7 | 12 | B |
| ILE | C | 216 | 43.0 | 25.7 | 41.1 | 3 | B |
| ILE | O | 216 | 43.7 | 26.0 | 42.0 | 4 | B |
| GLY | N | 217 | 41.8 | 26.2 | 40.8 | 3 | B |
| GLY | CA | 217 | 41.2 | 27.2 | 41.7 | 1 | B |
| GLY | C | 217 | 41.5 | 28.7 | 41.6 | 1 | B |
| GLY | O | 217 | 41.5 | 29.4 | 42.6 | 1 | B |
| GLY | N | 218 | 41.7 | 29.1 | 40.4 | 1 | B |
| GLY | CA | 218 | 42.0 | 30.6 | 40.2 | 4 | B |
| GLY | C | 218 | 43.0 | 30.9 | 39.1 | 5 | B |
| GLY | O | 218 | 43.4 | 30.1 | 38.4 | 3 | B |
| ILE | N | 219 | 43.2 | 32.2 | 39.0 | 5 | B |
| ILE | CA | 219 | 44.1 | 32.7 | 37.9 | 3 | B |
| ILE | CB | 219 | 43.4 | 33.9 | 37.3 | 1 | B |
| ILE | CG2 | 219 | 44.0 | 34.1 | 35.9 | 3 | B |
| ILE | CG1 | 219 | 41.9 | 33.6 | 37.1 | 1 | B |
| ILE | CD1 | 219 | 41.1 | 34.8 | 36.7 | 1 | B |
| ILE | C | 219 | 45.5 | 33.1 | 38.4 | 3 | B |
| ILE | O | 219 | 45.6 | 33.6 | 39.5 | 4 | B |
| ASP | N | 220 | 46.5 | 33.0 | 37.5 | 6 | B |
| ASP | CA | 220 | 47.8 | 33.3 | 37.8 | 7 | B |
| ASP | CB | 220 | 48.8 | 32.1 | 37.8 | 8 | B |
| ASP | CG | 220 | 50.2 | 32.5 | 38.1 | 9 | B |
| ASP | OD1 | 220 | 50.7 | 32.0 | 39.1 | 9 | B |
| ASP | OD2 | 220 | 50.8 | 33.2 | 37.4 | 16 | B |
| ASP | C | 220 | 48.3 | 34.4 | 36.9 | 8 | B |
| ASP | O | 220 | 48.8 | 34.1 | 35.8 | 9 | B |
| HIS | N | 221 | 48.2 | 35.7 | 37.3 | 10 | B |
| HIS | CA | 221 | 48.5 | 36.8 | 36.4 | 12 | B |
| HIS | CB | 221 | 48.4 | 38.1 | 37.2 | 15 | B |
| HIS | CG | 221 | 47.0 | 38.4 | 37.5 | 23 | B |
| HIS | CD2 | 221 | 46.1 | 37.9 | 38.4 | 26 | B |
| HIS | ND1 | 221 | 46.3 | 39.5 | 36.9 | 23 | B |
| HIS | CE1 | 221 | 45.1 | 39.5 | 37.4 | 25 | B |
| HIS | NE2 | 221 | 44.9 | 38.6 | 38.2 | 28 | B |
| HIS | C | 221 | 49.9 | 36.7 | 35.8 | 11 | B |
| HIS | O | 221 | 50.2 | 37.5 | 34.9 | 14 | B |
| SER | N | 222 | 50.8 | 35.8 | 36.2 | 8 | B |
| SER | CA | 222 | 52.1 | 35.7 | 35.7 | 6 | B |
| SER | CB | 222 | 53.1 | 35.3 | 36.7 | 7 | B |
| SER | OG | 222 | 53.1 | 33.9 | 37.0 | 17 | B |
| SER | C | 222 | 52.2 | 34.7 | 34.5 | 3 | B |
| SER | O | 222 | 53.2 | 34.3 | 34.1 | 3 | B |
| LEU | N | 223 | 51.0 | 34.4 | 33.9 | 4 | B |
| LEU | CA | 223 | 50.9 | 33.5 | 32.8 | 5 | B |
| LEU | CB | 223 | 50.1 | 32.3 | 33.2 | 4 | B |
| LEU | CG | 223 | 50.7 | 31.4 | 34.3 | 1 | B |
| LEU | CD1 | 223 | 49.6 | 30.5 | 34.9 | 5 | B |
| LEU | CD2 | 223 | 51.8 | 30.6 | 33.8 | 1 | B |
| LEU | C | 223 | 50.4 | 34.2 | 31.6 | 6 | B |
| LEU | O | 223 | 50.4 | 33.6 | 30.5 | 8 | B |
| TYR | N | 224 | 49.9 | 35.4 | 31.7 | 3 | B |
| TYR | CA | 224 | 49.4 | 36.1 | 30.6 | 4 | B |
| TYR | CB | 224 | 47.9 | 35.9 | 30.5 | 6 | B |
| TYR | CG | 224 | 47.0 | 36.6 | 31.5 | 5 | B |
| TYR | CD1 | 224 | 47.0 | 36.2 | 32.8 | 4 | B |
| TYR | CE1 | 224 | 46.2 | 36.8 | 33.8 | 4 | B |
| TYR | CD2 | 224 | 46.2 | 37.7 | 31.2 | 2 | B |
| TYR | CE2 | 224 | 45.4 | 38.3 | 32.1 | 5 | B |
| TYR | CZ | 224 | 45.4 | 37.8 | 33.4 | 8 | B |
| TYR | OH | 224 | 44.5 | 38.4 | 34.4 | 8 | B |
| TYR | C | 224 | 49.7 | 37.6 | 30.5 | 4 | B |
| TYR | O | 224 | 49.8 | 38.2 | 31.5 | 2 | B |
| THR | N | 225 | 49.8 | 38.1 | 29.3 | 4 | B |
| THR | CA | 225 | 50.1 | 39.5 | 29.1 | 5 | B |
| THR | CB | 225 | 51.3 | 39.7 | 28.1 | 5 | B |
| THR | OG1 | 225 | 51.0 | 39.0 | 26.9 | 9 | B |
| THR | CG2 | 225 | 52.5 | 39.2 | 28.8 | 1 | B |
| THR | C | 225 | 48.8 | 40.1 | 28.4 | 6 | B |
| THR | O | 225 | 48.2 | 39.4 | 27.6 | 8 | B |
| GLY | N | 226 | 48.5 | 41.3 | 28.8 | 6 | B |
| GLY | CA | 226 | 47.3 | 42.0 | 28.2 | 8 | B |
| GLY | C | 226 | 46.1 | 41.8 | 29.1 | 5 | B |
| GLY | O | 226 | 46.3 | 41.5 | 30.3 | 2 | B |
| SER | N | 227 | 44.9 | 41.9 | 28.6 | 5 | B |
| SER | CA | 227 | 43.7 | 41.7 | 29.4 | 5 | B |
| SER | CB | 227 | 42.7 | 42.9 | 29.0 | 5 | B |
| SER | OG | 227 | 43.4 | 44.1 | 29.2 | 8 | B |
| SER | C | 227 | 43.0 | 40.4 | 29.1 | 3 | B |
| SER | O | 227 | 43.4 | 39.6 | 28.3 | 3 | B |
| LEU | N | 228 | 42.0 | 40.1 | 29.9 | 1 | B |
| LEU | CA | 228 | 41.2 | 38.9 | 29.8 | 1 | B |
| LEU | CB | 228 | 41.1 | 38.2 | 31.2 | 1 | B |
| LEU | CG | 228 | 42.2 | 37.3 | 31.7 | 1 | B |
| LEU | CD1 | 228 | 41.9 | 36.8 | 33.1 | 1 | B |
| LEU | CD2 | 228 | 42.4 | 36.2 | 30.8 | 1 | B |
| LEU | C | 228 | 39.8 | 39.2 | 29.3 | 1 | B |
| LEU | O | 228 | 39.1 | 39.8 | 30.0 | 1 | B |
| TRP | N | 229 | 39.5 | 38.6 | 28.1 | 1 | B |
| TRP | CA | 229 | 38.2 | 38.8 | 27.6 | 1 | B |
| TRP | CB | 229 | 38.2 | 39.2 | 26.1 | 1 | B |
| TRP | CG | 229 | 38.8 | 40.6 | 25.9 | 1 | B |
| TRP | CD2 | 229 | 38.0 | 41.7 | 25.5 | 1 | B |
| TRP | CE2 | 229 | 39.0 | 42.8 | 25.4 | 1 | B |
| TRP | CE3 | 229 | 36.7 | 42.0 | 25.3 | 1 | B |
| TRP | CD1 | 229 | 40.1 | 40.9 | 25.9 | 2 | B |
| TRP | NE1 | 229 | 40.2 | 42.3 | 25.6 | 1 | B |
| TRP | CZ2 | 229 | 38.6 | 44.1 | 25.0 | 1 | B |
| TRP | CZ3 | 229 | 36.3 | 43.3 | 25.0 | 3 | B |
| TRP | CH2 | 229 | 37.3 | 44.3 | 24.9 | 1 | B |
| TRP | C | 229 | 37.4 | 37.5 | 27.7 | 1 | B |
| TRP | O | 229 | 37.8 | 36.5 | 27.3 | 1 | B |
| TYR | N | 230 | 36.2 | 37.6 | 28.3 | 1 | B |
| TYR | CA | 230 | 35.4 | 36.5 | 28.5 | 1 | B |
| TYR | CB | 230 | 34.7 | 36.5 | 29.9 | 1 | B |
| TYR | CG | 230 | 35.7 | 36.5 | 31.0 | 1 | B |
| TYR | CD1 | 230 | 36.4 | 37.6 | 31.5 | 1 | B |
| TYR | CE1 | 230 | 37.3 | 37.6 | 32.5 | 1 | B |
| TYR | CD2 | 230 | 36.1 | 35.3 | 31.6 | 1 | B |
| TYR | CE2 | 230 | 37.1 | 35.2 | 32.6 | 2 | B |
| TYR | CZ | 230 | 37.7 | 36.4 | 33.0 | 1 | B |
| TYR | OH | 230 | 38.6 | 36.3 | 34.0 | 1 | B |
| TYR | C | 230 | 34.3 | 36.2 | 27.5 | 4 | B |
| TYR | O | 230 | 33.8 | 37.2 | 26.9 | 5 | B |
| THR | N | 231 | 33.9 | 35.0 | 27.3 | 3 | B |
| THR | CA | 231 | 32.9 | 34.6 | 26.4 | 2 | B |
| THR | CB | 231 | 33.4 | 33.9 | 25.2 | 1 | B |
| THR | OG1 | 231 | 32.4 | 33.4 | 24.3 | 1 | B |
| THR | CG2 | 231 | 34.2 | 32.6 | 25.6 | 1 | B |
| THR | C | 231 | 32.0 | 33.6 | 27.2 | 3 | B |
| THR | O | 231 | 32.4 | 32.8 | 27.9 | 1 | B |
| PRO | N | 232 | 30.6 | 33.8 | 27.0 | 2 | B |
| PRO | CD | 232 | 30.0 | 34.8 | 26.2 | 2 | B |
| PRO | CA | 232 | 29.6 | 33.0 | 27.7 | 2 | B |
| PRO | CB | 232 | 28.3 | 33.6 | 27.1 | 1 | B |
| PRO | CG | 232 | 28.7 | 35.0 | 26.8 | 1 | B |
| PRO | C | 232 | 29.7 | 31.5 | 27.5 | 2 | B |
| PRO | O | 232 | 30.0 | 31.1 | 26.3 | 1 | B |
| ILE | N | 233 | 29.5 | 30.7 | 28.5 | 2 | B |
| ILE | CA | 233 | 29.5 | 29.3 | 28.3 | 1 | B |
| ILE | CB | 233 | 29.8 | 28.5 | 29.6 | 1 | B |
| ILE | CG2 | 233 | 29.7 | 27.0 | 29.3 | 1 | B |
| ILE | CG1 | 233 | 31.3 | 28.7 | 30.0 | 1 | B |
| ILE | CD1 | 233 | 31.8 | 27.8 | 31.1 | 1 | B |
| ILE | C | 233 | 28.1 | 29.0 | 28.0 | 1 | B |
| ILE | O | 233 | 27.2 | 29.1 | 28.8 | 1 | B |
| ARG | N | 234 | 27.8 | 28.6 | 26.7 | 1 | B |
| ARG | CA | 234 | 26.5 | 28.3 | 26.3 | 1 | B |
| ARG | CB | 234 | 26.5 | 27.7 | 24.9 | 1 | B |
| ARG | CG | 234 | 25.8 | 28.6 | 23.9 | 1 | B |
| ARG | CD | 234 | 25.2 | 27.8 | 22.8 | 1 | B |
| ARG | NE | 234 | 23.9 | 28.3 | 22.4 | 1 | B |
| ARG | CZ | 234 | 23.1 | 27.7 | 21.6 | 2 | B |
| ARG | NH1 | 234 | 23.3 | 26.5 | 21.1 | 1 | B |
| ARG | NH2 | 234 | 21.9 | 28.3 | 21.3 | 9 | B |
| ARG | C | 234 | 25.7 | 27.4 | 27.2 | 1 | B |
| ARG | O | 234 | 24.8 | 27.9 | 27.9 | 2 | B |
| ARG | N | 235 | 25.9 | 26.1 | 27.1 | 1 | B |
| ARG | CA | 235 | 25.2 | 25.2 | 27.9 | 1 | B |
| ARG | CB | 235 | 24.4 | 24.2 | 27.0 | 3 | B |
| ARG | CG | 235 | 23.2 | 23.5 | 27.8 | 4 | B |
| ARG | CD | 235 | 23.1 | 22.1 | 27.5 | 1 | B |
| ARG | NE | 235 | 23.0 | 21.8 | 26.0 | 1 | B |
| ARG | CZ | 235 | 23.0 | 20.6 | 25.5 | 2 | B |
| ARG | NH1 | 235 | 23.0 | 19.5 | 26.4 | 1 | B |
| ARG | NH2 | 235 | 22.9 | 20.4 | 24.2 | 2 | B |
| ARG | C | 235 | 26.3 | 24.4 | 28.7 | 1 | B |
| ARG | O | 235 | 27.3 | 24.0 | 28.1 | 1 | B |
| GLU | N | 236 | 26.0 | 24.2 | 30.0 | 1 | B |
| GLU | CA | 236 | 27.0 | 23.5 | 30.8 | 2 | B |
| GLU | CB | 236 | 26.8 | 23.7 | 32.3 | 3 | B |
| GLU | CG | 236 | 27.0 | 25.2 | 32.7 | 12 | B |
| GLU | CD | 236 | 27.0 | 25.4 | 34.2 | 13 | B |
| GLU | OE1 | 236 | 26.0 | 25.1 | 34.8 | 18 | B |
| GLU | OE2 | 236 | 28.1 | 25.8 | 34.7 | 13 | B |
| GLU | C | 236 | 27.1 | 22.0 | 30.5 | 3 | B |
| GLU | O | 236 | 26.7 | 21.2 | 31.4 | 9 | B |
| TRP | N | 237 | 27.6 | 21.6 | 29.4 | 1 | B |
| TRP | CA | 237 | 27.8 | 20.2 | 29.1 | 1 | B |
| TRP | CB | 237 | 26.8 | 19.6 | 28.0 | 1 | B |
| TRP | CG | 237 | 26.7 | 20.4 | 26.8 | 1 | B |
| TRP | CD2 | 237 | 26.4 | 19.8 | 25.5 | 1 | B |
| TRP | CE2 | 237 | 26.3 | 20.9 | 24.6 | 1 | B |
| TRP | CE3 | 237 | 26.1 | 18.5 | 25.1 | 1 | B |
| TRP | CD1 | 237 | 26.8 | 21.7 | 26.6 | 4 | B |
| TRP | NE1 | 237 | 26.6 | 22.0 | 25.2 | 1 | B |
| TRP | CZ2 | 237 | 26.0 | 20.7 | 23.2 | 2 | B |
| TRP | CZ3 | 237 | 25.8 | 18.3 | 23.7 | 1 | B |
| TRP | CH2 | 237 | 25.7 | 19.4 | 22.8 | 1 | B |
| TRP | C | 237 | 29.2 | 20.1 | 28.6 | 1 | B |
| TRP | O | 237 | 30.1 | 19.6 | 29.4 | 1 | B |
| TYR | N | 238 | 29.5 | 20.4 | 27.4 | 1 | B |
| TYR | CA | 238 | 30.8 | 20.5 | 26.9 | 1 | B |
| TYR | CB | 238 | 30.9 | 20.3 | 25.4 | 1 | B |
| TYR | CG | 238 | 30.8 | 18.9 | 24.9 | 1 | B |
| TYR | CD1 | 238 | 31.9 | 18.0 | 25.0 | 2 | B |
| TYR | CE1 | 238 | 31.9 | 16.7 | 24.4 | 3 | B |
| TYR | CD2 | 238 | 29.7 | 18.4 | 24.2 | 1 | B |
| TYR | CE2 | 238 | 29.6 | 17.2 | 23.6 | 1 | B |
| TYR | CZ | 238 | 30.7 | 16.3 | 23.7 | 3 | B |
| TYR | OH | 238 | 30.7 | 15.1 | 23.1 | 4 | B |
| TYR | C | 238 | 31.1 | 21.9 | 27.2 | 1 | B |
| TYR | O | 238 | 30.1 | 22.6 | 27.5 | 1 | B |
| TYR | N | 239 | 32.3 | 22.4 | 27.2 | 1 | B |
| TYR | CA | 239 | 32.5 | 23.9 | 27.4 | 1 | B |
| TYR | CB | 239 | 33.9 | 24.2 | 27.8 | 1 | B |
| TYR | CG | 239 | 34.2 | 23.7 | 29.2 | 1 | B |
| TYR | CD1 | 239 | 34.9 | 22.5 | 29.5 | 1 | B |
| TYR | CE1 | 239 | 35.2 | 22.1 | 30.7 | 1 | B |
| TYR | CD2 | 239 | 33.9 | 24.5 | 30.3 | 2 | B |
| TYR | CE2 | 239 | 34.2 | 24.1 | 31.6 | 1 | B |
| TYR | CZ | 239 | 34.8 | 22.9 | 31.8 | 1 | B |
| TYR | OH | 239 | 35.1 | 22.5 | 33.1 | 1 | B |
| TYR | C | 239 | 32.1 | 24.6 | 26.1 | 1 | B |
| TYR | O | 239 | 33.0 | 25.1 | 25.5 | 1 | B |
| GLU | N | 240 | 30.9 | 24.6 | 25.8 | 1 | B |
| GLU | CA | 240 | 30.4 | 25.2 | 24.5 | 1 | B |
| GLU | CB | 240 | 29.0 | 24.7 | 24.2 | 1 | B |
| GLU | CG | 240 | 28.4 | 25.5 | 23.0 | 1 | B |
| GLU | CD | 240 | 27.0 | 24.9 | 22.5 | 1 | B |
| GLU | OE1 | 240 | 26.2 | 24.6 | 23.4 | 1 | B |
| GLU | OE2 | 240 | 26.9 | 24.8 | 21.3 | 1 | B |
| GLU | C | 240 | 30.5 | 26.7 | 24.5 | 1 | B |
| GLU | O | 240 | 30.3 | 27.3 | 25.5 | 1 | B |
| VAL | N | 241 | 30.9 | 27.2 | 23.3 | 1 | B |
| VAL | CA | 241 | 31.0 | 28.6 | 23.1 | 1 | B |
| VAL | CB | 241 | 32.5 | 29.1 | 23.1 | 1 | B |
| VAL | CG1 | 241 | 33.2 | 28.9 | 24.4 | 1 | B |
| VAL | CG2 | 241 | 33.2 | 28.3 | 22.0 | 1 | B |
| VAL | C | 241 | 30.4 | 28.9 | 21.8 | 1 | B |
| VAL | O | 241 | 30.1 | 28.0 | 21.1 | 1 | B |
| ILE | N | 242 | 30.2 | 30.2 | 21.4 | 1 | B |
| ILE | CA | 242 | 29.6 | 30.6 | 20.1 | 1 | B |
| ILE | CB | 242 | 28.3 | 31.3 | 20.4 | 1 | B |
| ILE | CG2 | 242 | 27.9 | 32.0 | 19.1 | 1 | B |
| ILE | CG1 | 242 | 27.2 | 30.4 | 20.9 | 1 | B |
| ILE | CD1 | 242 | 26.1 | 31.1 | 21.5 | 1 | B |
| ILE | C | 242 | 30.6 | 31.5 | 19.3 | 1 | B |
| ILE | O | 242 | 30.9 | 32.5 | 19.8 | 1 | B |
| ILE | N | 243 | 31.0 | 31.0 | 18.2 | 2 | B |
| ILE | CA | 243 | 31.8 | 31.8 | 17.3 | 1 | B |
| ILE | CB | 243 | 32.7 | 30.9 | 16.4 | 1 | B |
| ILE | CG2 | 243 | 33.4 | 31.7 | 15.3 | 1 | B |
| ILE | CG1 | 243 | 33.8 | 30.2 | 17.3 | 1 | B |
| ILE | CD1 | 243 | 34.7 | 29.3 | 16.5 | 1 | B |
| ILE | C | 243 | 30.9 | 32.7 | 16.4 | 1 | B |
| ILE | O | 243 | 30.0 | 32.2 | 15.9 | 1 | B |
| VAL | N | 244 | 31.2 | 34.0 | 16.4 | 1 | B |
| VAL | CA | 244 | 30.3 | 34.9 | 15.6 | 1 | B |
| VAL | CB | 244 | 29.9 | 36.1 | 16.4 | 1 | B |
| VAL | CG1 | 244 | 29.0 | 35.7 | 17.5 | 2 | B |
| VAL | CG2 | 244 | 31.1 | 36.8 | 16.9 | 1 | B |
| VAL | C | 244 | 31.0 | 35.4 | 14.3 | 1 | B |
| VAL | O | 244 | 30.3 | 36.1 | 13.6 | 3 | B |
| ARG | N | 245 | 32.2 | 35.0 | 14.1 | 1 | B |
| ARG | CA | 245 | 32.9 | 35.5 | 12.9 | 1 | B |
| ARG | CB | 245 | 33.1 | 37.0 | 13.0 | 1 | B |
| ARG | CG | 245 | 33.7 | 37.7 | 11.8 | 1 | B |
| ARG | CD | 245 | 34.2 | 39.1 | 12.2 | 2 | B |
| ARG | NE | 245 | 34.7 | 39.9 | 11.1 | 1 | B |
| ARG | CZ | 245 | 33.9 | 40.5 | 10.2 | 1 | B |
| ARG | NH1 | 245 | 32.6 | 40.2 | 10.2 | 1 | B |
| ARG | NH2 | 245 | 34.4 | 41.3 | 9.3 | 2 | B |
| ARG | C | 245 | 34.3 | 34.8 | 12.7 | 1 | B |
| ARG | O | 245 | 34.9 | 34.4 | 13.7 | 1 | B |
| VAL | N | 246 | 34.8 | 34.7 | 11.5 | 1 | B |
| VAL | CA | 246 | 36.1 | 34.1 | 11.2 | 1 | B |
| VAL | CB | 246 | 35.9 | 32.7 | 10.8 | 1 | B |
| VAL | CG1 | 246 | 37.3 | 32.1 | 10.4 | 1 | B |
| VAL | CG2 | 246 | 35.4 | 31.9 | 12.0 | 1 | B |
| VAL | C | 246 | 36.7 | 34.9 | 10.1 | 1 | B |
| VAL | O | 246 | 36.1 | 35.1 | 9.1 | 1 | B |
| GLU | N | 247 | 38.0 | 35.3 | 10.2 | 1 | B |
| GLU | CA | 247 | 38.7 | 36.0 | 9.2 | 1 | B |
| GLU | CB | 247 | 39.0 | 37.4 | 9.6 | 1 | B |
| GLU | CG | 247 | 38.0 | 38.1 | 10.5 | 2 | B |
| GLU | CD | 247 | 38.4 | 39.5 | 10.8 | 4 | B |
| GLU | OE1 | 247 | 39.7 | 39.8 | 11.0 | 6 | B |
| GLU | OE2 | 247 | 37.6 | 40.4 | 11.0 | 2 | B |
| GLU | C | 247 | 40.1 | 35.3 | 8.9 | 1 | B |
| GLU | O | 247 | 40.7 | 34.7 | 9.8 | 1 | B |
| ILE | N | 248 | 40.5 | 35.3 | 7.7 | 1 | B |
| ILE | CA | 248 | 41.8 | 34.8 | 7.3 | 1 | B |
| ILE | CB | 248 | 41.8 | 33.7 | 6.2 | 1 | B |
| ILE | CG2 | 248 | 43.1 | 33.1 | 6.0 | 1 | B |
| ILE | CG1 | 248 | 40.8 | 32.7 | 6.5 | 1 | B |
| ILE | CD1 | 248 | 41.1 | 31.9 | 7.8 | 1 | B |
| ILE | C | 248 | 42.6 | 36.0 | 6.9 | 1 | B |
| ILE | O | 248 | 42.4 | 36.5 | 5.7 | 1 | B |
| ASN | N | 249 | 43.5 | 36.5 | 7.7 | 3 | B |
| ASN | CA | 249 | 44.3 | 37.6 | 7.4 | 1 | B |
| ASN | CB | 249 | 45.2 | 37.4 | 6.2 | 1 | B |
| ASN | CG | 249 | 46.7 | 37.2 | 6.5 | 1 | B |
| ASN | OD1 | 249 | 47.0 | 36.9 | 7.6 | 1 | B |
| ASN | ND2 | 249 | 47.5 | 37.5 | 5.5 | 1 | B |
| ASN | C | 249 | 43.4 | 38.8 | 7.2 | 1 | B |
| ASN | O | 249 | 43.5 | 39.5 | 6.2 | 1 | B |
| GLY | N | 250 | 42.5 | 39.0 | 8.1 | 1 | B |
| GLY | CA | 250 | 41.6 | 40.1 | 8.1 | 1 | B |
| GLY | C | 250 | 40.4 | 39.9 | 7.1 | 1 | B |
| GLY | O | 250 | 39.4 | 40.7 | 7.2 | 3 | B |
| GLN | N | 251 | 40.5 | 39.0 | 6.2 | 2 | B |
| GLN | CA | 251 | 39.4 | 38.8 | 5.3 | 3 | B |
| GLN | CB | 251 | 40.0 | 38.1 | 4.0 | 7 | B |
| GLN | CG | 251 | 39.1 | 38.2 | 2.8 | 7 | B |
| GLN | CD | 251 | 39.9 | 38.6 | 1.5 | 11 | B |
| GLN | OE1 | 251 | 39.6 | 38.2 | 0.4 | 12 | B |
| GLN | NE2 | 251 | 40.9 | 39.4 | 1.7 | 13 | B |
| GLN | C | 251 | 38.3 | 37.9 | 5.9 | 1 | B |
| GLN | O | 251 | 38.5 | 36.8 | 6.2 | 1 | B |
| ASP | N | 252 | 37.1 | 38.5 | 6.0 | 1 | B |
| ASP | CA | 252 | 36.0 | 37.8 | 6.5 | 1 | B |
| ASP | CB | 252 | 34.8 | 38.8 | 6.7 | 1 | B |
| ASP | CG | 252 | 33.5 | 38.1 | 7.0 | 1 | B |
| ASP | OD1 | 252 | 33.6 | 37.0 | 7.6 | 1 | B |
| ASP | OD2 | 252 | 32.5 | 38.7 | 6.8 | 1 | B |
| ASP | C | 252 | 35.6 | 36.7 | 5.6 | 1 | B |
| ASP | O | 252 | 35.2 | 36.9 | 4.5 | 1 | B |
| LEU | N | 253 | 35.7 | 35.4 | 6.2 | 1 | B |
| LEU | CA | 253 | 35.3 | 34.2 | 5.5 | 1 | B |
| LEU | CB | 253 | 35.3 | 33.0 | 6.4 | 3 | B |
| LEU | CG | 253 | 35.9 | 31.7 | 6.0 | 1 | B |
| LEU | CD1 | 253 | 37.3 | 32.0 | 5.5 | 1 | B |
| LEU | CD2 | 253 | 36.0 | 30.8 | 7.2 | 1 | B |
| LEU | C | 253 | 33.9 | 34.4 | 4.9 | 4 | B |
| LEU | O | 253 | 33.5 | 33.7 | 3.9 | 7 | B |
| LYS | N | 254 | 33.2 | 35.4 | 5.4 | 4 | B |
| LYS | CA | 254 | 31.8 | 35.7 | 5.0 | 6 | B |
| LYS | CB | 254 | 31.9 | 36.4 | 3.7 | 9 | B |
| LYS | CG | 254 | 30.8 | 37.5 | 3.4 | 12 | B |
| LYS | CD | 254 | 30.9 | 38.0 | 2.0 | 14 | B |
| LYS | CE | 254 | 29.9 | 39.1 | 1.7 | 19 | B |
| LYS | NZ | 254 | 30.1 | 39.8 | 0.4 | 18 | B |
| LYS | C | 254 | 30.9 | 34.5 | 4.9 | 7 | B |
| LYS | O | 254 | 30.4 | 34.2 | 3.8 | 11 | B |
| MET | N | 255 | 30.7 | 33.8 | 6.0 | 6 | B |
| MET | CA | 255 | 29.8 | 32.7 | 6.0 | 4 | B |
| MET | CB | 255 | 30.5 | 31.4 | 6.5 | 4 | B |
| MET | CG | 255 | 30.9 | 30.5 | 5.3 | 1 | B |
| MET | SD | 255 | 32.1 | 29.3 | 5.7 | 2 | B |
| MET | CE | 255 | 31.2 | 28.0 | 6.3 | 4 | B |
| MET | C | 255 | 28.7 | 33.0 | 7.0 | 2 | B |
| MET | O | 255 | 28.8 | 34.0 | 7.8 | 1 | B |
| ASP | N | 256 | 27.6 | 32.3 | 7.0 | 1 | B |
| ASP | CA | 256 | 26.5 | 32.6 | 8.0 | 1 | B |
| ASP | CB | 256 | 25.3 | 31.8 | 7.5 | 6 | B |
| ASP | CG | 256 | 24.2 | 31.7 | 8.5 | 9 | B |
| ASP | OD1 | 256 | 24.4 | 30.9 | 9.5 | 11 | B |
| ASP | OD2 | 256 | 23.2 | 32.3 | 8.4 | 13 | B |
| ASP | C | 256 | 26.9 | 32.2 | 9.4 | 1 | B |
| ASP | O | 256 | 27.3 | 31.1 | 9.7 | 1 | B |
| CYS | N | 257 | 26.7 | 33.2 | 10.3 | 1 | B |
| CYS | CA | 257 | 27.0 | 33.0 | 11.7 | 1 | B |
| CYS | CB | 257 | 26.1 | 33.9 | 12.6 | 1 | B |
| CYS | SG | 257 | 26.3 | 34.0 | 14.5 | 7 | B |
| CYS | C | 257 | 26.9 | 31.5 | 12.1 | 1 | B |
| CYS | O | 257 | 27.9 | 31.0 | 12.7 | 1 | B |
| LYS | N | 258 | 25.8 | 30.9 | 11.8 | 1 | B |
| LYS | CA | 258 | 25.6 | 29.5 | 12.2 | 1 | B |
| LYS | CB | 258 | 24.1 | 29.1 | 11.8 | 5 | B |
| LYS | CG | 258 | 23.0 | 29.9 | 12.5 | 9 | B |
| LYS | CD | 258 | 21.7 | 29.1 | 12.6 | 16 | B |
| LYS | CE | 258 | 20.6 | 29.9 | 13.1 | 19 | B |
| LYS | NZ | 258 | 20.1 | 30.9 | 12.1 | 24 | B |
| LYS | C | 258 | 26.5 | 28.4 | 11.7 | 2 | B |
| LYS | O | 258 | 26.7 | 27.4 | 12.3 | 4 | B |
| GLU | N | 259 | 27.1 | 28.6 | 10.5 | 1 | B |
| GLU | CA | 259 | 28.0 | 27.5 | 10.0 | 1 | B |
| GLU | CB | 259 | 28.5 | 27.9 | 8.6 | 5 | B |
| GLU | CG | 259 | 27.5 | 27.4 | 7.5 | 4 | B |
| GLU | CD | 259 | 27.2 | 26.0 | 7.5 | 8 | B |
| GLU | OE1 | 259 | 28.0 | 25.2 | 8.1 | 7 | B |
| GLU | OE2 | 259 | 26.1 | 25.6 | 7.0 | 11 | B |
| GLU | C | 259 | 29.3 | 27.2 | 10.9 | 1 | B |
| GLU | O | 259 | 29.7 | 26.1 | 11.0 | 1 | B |
| TYR | N | 260 | 29.8 | 28.3 | 11.5 | 1 | B |
| TYR | CA | 260 | 31.0 | 28.1 | 12.3 | 1 | B |
| TYR | CB | 260 | 31.6 | 29.5 | 12.7 | 1 | B |
| TYR | CG | 260 | 31.9 | 30.4 | 11.6 | 1 | B |
| TYR | CD1 | 260 | 31.6 | 31.8 | 11.8 | 1 | B |
| TYR | CE1 | 260 | 31.8 | 32.7 | 10.8 | 1 | B |
| TYR | CD2 | 260 | 32.4 | 30.0 | 10.3 | 1 | B |
| TYR | CE2 | 260 | 32.6 | 31.0 | 9.3 | 1 | B |
| TYR | CZ | 260 | 32.4 | 32.3 | 9.6 | 1 | B |
| TYR | OH | 260 | 32.6 | 33.2 | 8.6 | 5 | B |
| TYR | C | 260 | 30.7 | 27.3 | 13.5 | 1 | B |
| TYR | O | 260 | 31.5 | 26.7 | 14.2 | 1 | B |
| ASN | N | 261 | 29.4 | 27.4 | 13.9 | 1 | B |
| ASN | CA | 261 | 28.9 | 26.7 | 15.2 | 1 | B |
| ASN | CB | 261 | 28.3 | 27.7 | 16.1 | 1 | B |
| ASN | CG | 261 | 29.1 | 28.9 | 16.4 | 1 | B |
| ASN | OD1 | 261 | 30.2 | 28.8 | 16.9 | 1 | B |
| ASN | ND2 | 261 | 28.6 | 30.1 | 16.1 | 1 | B |
| ASN | C | 261 | 28.0 | 25.6 | 14.9 | 2 | B |
| ASN | O | 261 | 27.1 | 25.3 | 15.8 | 1 | B |
| TYR | N | 262 | 28.0 | 25.0 | 13.7 | 8 | B |
| TYR | CA | 262 | 27.2 | 24.0 | 13.3 | 10 | B |
| TYR | CB | 262 | 27.4 | 23.6 | 11.8 | 13 | B |
| TYR | CG | 262 | 26.6 | 22.4 | 11.4 | 22 | B |
| TYR | CD1 | 262 | 25.2 | 22.4 | 11.8 | 25 | B |
| TYR | CE1 | 262 | 24.5 | 21.3 | 11.4 | 30 | B |
| TYR | CD2 | 262 | 27.2 | 21.3 | 10.7 | 26 | B |
| TYR | CE2 | 262 | 26.4 | 20.2 | 10.4 | 24 | B |
| TYR | CZ | 262 | 25.0 | 20.2 | 10.7 | 30 | B |
| TYR | OH | 262 | 24.2 | 19.2 | 10.4 | 30 | B |
| TYR | C | 262 | 27.2 | 22.7 | 14.1 | 10 | B |
| TYR | O | 262 | 28.2 | 21.9 | 14.0 | 9 | B |
| ASP | N | 263 | 26.2 | 22.6 | 15.0 | 8 | B |
| ASP | CA | 263 | 26.1 | 21.5 | 16.0 | 8 | B |
| ASP | CB | 263 | 26.9 | 20.2 | 15.6 | 16 | B |
| ASP | CG | 263 | 26.6 | 19.0 | 16.4 | 20 | B |
| ASP | OD1 | 263 | 25.5 | 18.9 | 16.9 | 25 | B |
| ASP | OD2 | 263 | 27.5 | 18.2 | 16.6 | 23 | B |
| ASP | C | 263 | 26.6 | 22.1 | 17.2 | 4 | B |
| ASP | O | 263 | 25.8 | 22.4 | 18.1 | 2 | B |
| LYS | N | 264 | 27.9 | 22.3 | 17.3 | 3 | B |
| LYS | CA | 264 | 28.4 | 22.9 | 18.5 | 3 | B |
| LYS | CB | 264 | 28.4 | 21.9 | 19.7 | 5 | B |
| LYS | CG | 264 | 29.2 | 20.7 | 19.4 | 9 | B |
| LYS | CD | 264 | 29.3 | 19.8 | 20.6 | 9 | B |
| LYS | CE | 264 | 30.0 | 18.5 | 20.3 | 11 | B |
| LYS | NZ | 264 | 29.1 | 17.6 | 19.5 | 11 | B |
| LYS | C | 264 | 29.9 | 23.4 | 18.3 | 2 | B |
| LYS | O | 264 | 30.6 | 23.1 | 17.4 | 2 | B |
| SER | N | 265 | 30.3 | 24.3 | 19.3 | 1 | B |
| SER | CA | 265 | 31.6 | 24.9 | 19.3 | 1 | B |
| SER | CB | 265 | 31.6 | 26.3 | 18.9 | 3 | B |
| SER | OG | 265 | 31.4 | 26.5 | 17.5 | 3 | B |
| SER | C | 265 | 32.1 | 24.7 | 20.7 | 1 | B |
| SER | O | 265 | 31.4 | 25.2 | 21.7 | 2 | B |
| ILE | N | 266 | 33.2 | 24.1 | 20.9 | 1 | B |
| ILE | CA | 266 | 33.7 | 23.8 | 22.2 | 1 | B |
| ILE | CB | 266 | 33.5 | 22.3 | 22.6 | 2 | B |
| ILE | CG2 | 266 | 32.0 | 22.0 | 22.5 | 5 | B |
| ILE | CG1 | 266 | 34.3 | 21.4 | 21.6 | 1 | B |
| ILE | CD1 | 266 | 34.0 | 19.9 | 21.7 | 1 | B |
| ILE | C | 266 | 35.2 | 24.0 | 22.4 | 1 | B |
| ILE | O | 266 | 36.0 | 24.0 | 21.4 | 6 | B |
| VAL | N | 267 | 35.7 | 24.2 | 23.6 | 1 | B |
| VAL | CA | 267 | 37.1 | 24.4 | 23.9 | 1 | B |
| VAL | CB | 267 | 37.3 | 25.6 | 24.9 | 1 | B |
| VAL | CG1 | 267 | 38.7 | 25.8 | 25.1 | 1 | B |
| VAL | CG2 | 267 | 36.7 | 26.8 | 24.3 | 1 | B |
| VAL | C | 267 | 37.5 | 23.1 | 24.5 | 2 | B |
| VAL | O | 267 | 37.3 | 22.9 | 25.7 | 4 | B |
| ASP | N | 268 | 38.1 | 22.2 | 23.8 | 3 | B |
| ASP | CA | 268 | 38.6 | 20.9 | 24.2 | 1 | B |
| ASP | CB | 268 | 37.9 | 19.9 | 23.3 | 1 | B |
| ASP | CG | 268 | 38.5 | 18.5 | 23.4 | 1 | B |
| ASP | OD1 | 268 | 38.4 | 17.9 | 24.5 | 1 | B |
| ASP | OD2 | 268 | 39.0 | 18.0 | 22.4 | 4 | B |
| ASP | C | 268 | 40.1 | 20.7 | 24.2 | 1 | B |
| ASP | O | 268 | 40.7 | 20.6 | 23.1 | 1 | B |
| SER | N | 269 | 40.7 | 20.6 | 25.3 | 1 | B |
| SER | CA | 269 | 42.2 | 20.5 | 25.4 | 1 | B |
| SER | CB | 269 | 42.7 | 20.9 | 26.7 | 2 | B |
| SER | OG | 269 | 42.4 | 19.9 | 27.7 | 5 | B |
| SER | C | 269 | 42.6 | 19.0 | 25.1 | 1 | B |
| SER | O | 269 | 43.8 | 18.7 | 25.0 | 2 | B |
| GLY | N | 270 | 41.6 | 18.2 | 24.8 | 1 | B |
| GLY | CA | 270 | 41.9 | 16.8 | 24.6 | 1 | B |
| GLY | C | 270 | 42.3 | 16.5 | 23.1 | 2 | B |
| GLY | O | 270 | 42.9 | 15.5 | 22.8 | 5 | B |
| THR | N | 271 | 41.9 | 17.4 | 22.2 | 4 | B |
| THR | CA | 271 | 42.2 | 17.3 | 20.8 | 8 | B |
| THR | CB | 271 | 41.0 | 17.7 | 19.9 | 10 | B |
| THR | OG1 | 271 | 39.8 | 17.1 | 20.5 | 12 | B |
| THR | CG2 | 271 | 41.2 | 17.1 | 18.5 | 18 | B |
| THR | C | 271 | 43.4 | 18.2 | 20.4 | 6 | B |
| THR | O | 271 | 43.5 | 19.3 | 20.9 | 10 | B |
| THR | N | 272 | 44.2 | 17.7 | 19.5 | 4 | B |
| THR | CA | 272 | 45.3 | 18.5 | 19.0 | 1 | B |
| THR | CB | 272 | 46.4 | 17.6 | 18.3 | 3 | B |
| THR | OG1 | 272 | 46.7 | 16.6 | 19.2 | 5 | B |
| THR | CG2 | 272 | 47.6 | 18.4 | 17.9 | 5 | B |
| THR | C | 272 | 45.0 | 19.7 | 18.1 | 1 | B |
| THR | O | 272 | 45.2 | 20.8 | 18.4 | 1 | B |
| ASN | N | 273 | 44.5 | 19.3 | 16.9 | 3 | B |
| ASN | CA | 273 | 44.1 | 20.2 | 15.8 | 1 | B |
| ASN | CB | 273 | 43.8 | 19.4 | 14.6 | 1 | B |
| ASN | CG | 273 | 44.9 | 18.7 | 14.1 | 1 | B |
| ASN | OD1 | 273 | 45.9 | 18.5 | 14.7 | 1 | B |
| ASN | ND2 | 273 | 44.8 | 18.2 | 12.8 | 1 | B |
| ASN | C | 273 | 43.0 | 21.2 | 16.1 | 1 | B |
| ASN | O | 273 | 42.3 | 21.0 | 17.1 | 1 | B |
| LEU | N | 274 | 42.8 | 22.2 | 15.3 | 1 | B |
| LEU | CA | 274 | 41.7 | 23.1 | 15.4 | 1 | B |
| LEU | CB | 274 | 42.0 | 24.5 | 14.9 | 3 | B |
| LEU | CG | 274 | 40.9 | 25.6 | 14.8 | 1 | B |
| LEU | CD1 | 274 | 41.2 | 26.5 | 13.7 | 1 | B |
| LEU | CD2 | 274 | 39.5 | 25.1 | 14.7 | 1 | B |
| LEU | C | 274 | 40.8 | 22.4 | 14.4 | 1 | B |
| LEU | O | 274 | 41.1 | 22.2 | 13.3 | 1 | B |
| ARG | N | 275 | 39.6 | 22.0 | 14.9 | 1 | B |
| ARG | CA | 275 | 38.7 | 21.3 | 14.0 | 1 | B |
| ARG | CB | 275 | 38.2 | 20.0 | 14.7 | 1 | B |
| ARG | CG | 275 | 39.2 | 18.9 | 14.7 | 1 | B |
| ARG | CD | 275 | 38.9 | 17.8 | 15.7 | 6 | B |
| ARG | NE | 275 | 37.6 | 17.1 | 15.5 | 15 | B |
| ARG | CZ | 275 | 37.3 | 16.3 | 14.5 | 23 | B |
| ARG | NH1 | 275 | 36.0 | 15.8 | 14.4 | 22 | B |
| ARG | NH2 | 275 | 38.2 | 16.1 | 13.5 | 26 | B |
| ARG | C | 275 | 37.5 | 22.2 | 13.5 | 1 | B |
| ARG | O | 275 | 36.9 | 22.9 | 14.4 | 2 | B |
| LEU | N | 276 | 37.3 | 22.3 | 12.2 | 1 | B |
| LEU | CA | 276 | 36.3 | 23.1 | 11.7 | 1 | B |
| LEU | CB | 276 | 36.9 | 24.1 | 10.7 | 1 | B |
| LEU | CG | 276 | 37.9 | 25.2 | 11.3 | 1 | B |
| LEU | CD1 | 276 | 38.3 | 26.1 | 10.2 | 1 | B |
| LEU | CD2 | 276 | 37.2 | 25.9 | 12.4 | 4 | B |
| LEU | C | 276 | 35.2 | 22.3 | 10.9 | 3 | B |
| LEU | O | 276 | 35.5 | 21.3 | 10.3 | 2 | B |
| PRO | N | 277 | 34.0 | 22.7 | 11.0 | 5 | B |
| PRO | CD | 277 | 33.4 | 23.7 | 11.9 | 6 | B |
| PRO | CA | 277 | 32.9 | 22.0 | 10.3 | 8 | B |
| PRO | CB | 277 | 31.7 | 22.9 | 10.5 | 9 | B |
| PRO | CG | 277 | 31.9 | 23.3 | 11.9 | 4 | B |
| PRO | C | 277 | 33.3 | 21.9 | 8.8 | 9 | B |
| PRO | O | 277 | 33.8 | 22.8 | 8.2 | 6 | B |
| LYS | N | 278 | 32.9 | 20.7 | 8.2 | 12 | B |
| LYS | CA | 278 | 33.2 | 20.4 | 6.9 | 14 | B |
| LYS | CB | 278 | 32.4 | 19.1 | 6.4 | 15 | B |
| LYS | CG | 278 | 32.8 | 18.6 | 5.1 | 22 | B |
| LYS | CD | 278 | 34.4 | 18.5 | 5.0 | 27 | B |
| LYS | CE | 278 | 34.9 | 18.2 | 3.6 | 29 | B |
| LYS | NZ | 278 | 36.4 | 18.2 | 3.4 | 26 | B |
| LYS | C | 278 | 33.0 | 21.5 | 5.9 | 13 | B |
| LYS | O | 278 | 33.6 | 21.6 | 4.8 | 11 | B |
| LYS | N | 279 | 32.1 | 22.5 | 6.2 | 14 | B |
| LYS | CA | 279 | 31.8 | 23.6 | 5.3 | 15 | B |
| LYS | CB | 279 | 30.4 | 24.1 | 5.5 | 21 | B |
| LYS | CG | 279 | 29.3 | 23.1 | 5.3 | 27 | B |
| LYS | CD | 279 | 28.5 | 22.9 | 6.6 | 29 | B |
| LYS | CE | 279 | 27.2 | 22.2 | 6.4 | 29 | B |
| LYS | NZ | 279 | 26.4 | 22.1 | 7.6 | 29 | B |
| LYS | C | 279 | 32.8 | 24.7 | 5.5 | 12 | B |
| LYS | O | 279 | 33.3 | 25.3 | 4.6 | 14 | B |
| VAL | N | 280 | 33.1 | 24.9 | 6.8 | 8 | B |
| VAL | CA | 280 | 34.1 | 26.0 | 7.2 | 4 | B |
| VAL | CB | 280 | 33.9 | 26.3 | 8.7 | 3 | B |
| VAL | CG1 | 280 | 34.8 | 27.4 | 9.2 | 3 | B |
| VAL | CG2 | 280 | 32.4 | 26.7 | 9.0 | 1 | B |
| VAL | C | 280 | 35.5 | 25.6 | 6.9 | 3 | B |
| VAL | O | 280 | 36.3 | 26.5 | 6.8 | 4 | B |
| PHE | N | 281 | 35.8 | 24.3 | 6.9 | 2 | B |
| PHE | CA | 281 | 37.2 | 23.9 | 6.6 | 1 | B |
| PHE | CB | 281 | 37.3 | 22.4 | 6.9 | 1 | B |
| PHE | CG | 281 | 38.7 | 21.9 | 6.5 | 1 | B |
| PHE | CD1 | 281 | 39.8 | 22.1 | 7.3 | 1 | B |
| PHE | CD2 | 281 | 38.8 | 21.1 | 5.4 | 1 | B |
| PHE | CE1 | 281 | 41.0 | 21.5 | 7.0 | 1 | B |
| PHE | CE2 | 281 | 40.0 | 20.6 | 5.1 | 1 | B |
| PHE | CZ | 281 | 41.1 | 20.8 | 5.9 | 1 | B |
| PHE | C | 281 | 37.6 | 24.2 | 5.2 | 1 | B |
| PHE | O | 281 | 38.7 | 24.5 | 4.9 | 1 | B |
| GLU | N | 282 | 36.6 | 23.9 | 4.3 | 3 | B |
| GLU | CA | 282 | 36.8 | 24.1 | 2.9 | 5 | B |
| GLU | CB | 282 | 35.6 | 23.6 | 2.1 | 12 | B |
| GLU | CG | 282 | 35.2 | 22.2 | 2.4 | 18 | B |
| GLU | CD | 282 | 35.6 | 21.1 | 1.4 | 25 | B |
| GLU | OE1 | 282 | 35.0 | 21.1 | 0.3 | 29 | B |
| GLU | OE2 | 282 | 36.6 | 20.4 | 1.7 | 27 | B |
| GLU | C | 282 | 37.1 | 25.5 | 2.5 | 4 | B |
| GLU | O | 282 | 37.9 | 25.8 | 1.6 | 5 | B |
| ALA | N | 283 | 36.4 | 26.4 | 3.1 | 4 | B |
| ALA | CA | 283 | 36.5 | 27.9 | 2.9 | 4 | B |
| ALA | CB | 283 | 35.3 | 28.6 | 3.3 | 7 | B |
| ALA | C | 283 | 37.7 | 28.5 | 3.5 | 2 | B |
| ALA | O | 283 | 38.3 | 29.4 | 3.0 | 1 | B |
| ALA | N | 284 | 38.1 | 28.0 | 4.7 | 1 | B |
| ALA | CA | 284 | 39.2 | 28.5 | 5.5 | 1 | B |
| ALA | CB | 284 | 39.2 | 28.0 | 6.9 | 1 | B |
| ALA | C | 284 | 40.5 | 28.0 | 4.7 | 1 | B |
| ALA | O | 284 | 41.4 | 28.8 | 4.5 | 6 | B |
| VAL | N | 285 | 40.6 | 26.7 | 4.4 | 1 | B |
| VAL | CA | 285 | 41.7 | 26.2 | 3.7 | 2 | B |
| VAL | CB | 285 | 41.7 | 24.7 | 3.5 | 1 | B |
| VAL | CG1 | 285 | 42.8 | 24.2 | 2.6 | 1 | B |
| VAL | CG2 | 285 | 41.8 | 24.0 | 4.9 | 1 | B |
| VAL | C | 285 | 41.9 | 26.9 | 2.4 | 1 | B |
| VAL | O | 285 | 42.9 | 27.2 | 1.9 | 1 | B |
| LYS | N | 286 | 40.7 | 27.2 | 1.7 | 2 | B |
| LYS | CA | 286 | 40.7 | 27.8 | 0.4 | 3 | B |
| LYS | CB | 286 | 39.3 | 28.0 | −0.1 | 8 | B |
| LYS | CG | 286 | 39.2 | 28.4 | −1.5 | 13 | B |
| LYS | CD | 286 | 40.1 | 27.6 | −2.4 | 21 | B |
| LYS | CE | 286 | 40.0 | 26.1 | −2.3 | 23 | B |
| LYS | NZ | 286 | 41.2 | 25.3 | −2.8 | 17 | B |
| LYS | C | 286 | 41.4 | 29.1 | 0.5 | 1 | B |
| LYS | O | 286 | 42.2 | 29.5 | −0.3 | 1 | B |
| SER | N | 287 | 41.2 | 29.8 | 1.6 | 2 | B |
| SER | CA | 287 | 41.7 | 31.1 | 1.8 | 1 | B |
| SER | CB | 287 | 40.9 | 31.9 | 2.9 | 1 | B |
| SER | OG | 287 | 41.5 | 33.1 | 3.2 | 1 | B |
| SER | C | 287 | 43.2 | 31.0 | 2.3 | 1 | B |
| SER | O | 287 | 44.0 | 31.8 | 1.9 | 1 | B |
| ILE | N | 288 | 43.4 | 30.1 | 3.2 | 1 | B |
| ILE | CA | 288 | 44.8 | 29.9 | 3.7 | 1 | B |
| ILE | CB | 288 | 44.9 | 28.9 | 4.8 | 1 | B |
| ILE | CG2 | 288 | 46.3 | 28.7 | 5.3 | 1 | B |
| ILE | CG1 | 288 | 43.9 | 29.3 | 5.9 | 1 | B |
| ILE | CD1 | 288 | 43.8 | 28.3 | 7.0 | 1 | B |
| ILE | C | 288 | 45.7 | 29.5 | 2.6 | 1 | B |
| ILE | O | 288 | 46.8 | 30.0 | 2.4 | 1 | B |
| LYS | N | 289 | 45.2 | 28.6 | 1.7 | 2 | B |
| LYS | CA | 289 | 46.0 | 28.1 | 0.6 | 4 | B |
| LYS | CB | 289 | 45.1 | 27.1 | −0.2 | 3 | B |
| LYS | CG | 289 | 45.9 | 26.1 | −1.0 | 6 | B |
| LYS | CD | 289 | 45.1 | 25.0 | −1.7 | 4 | B |
| LYS | CE | 289 | 44.5 | 24.1 | −0.6 | 5 | B |
| LYS | NZ | 289 | 43.7 | 22.9 | −1.1 | 5 | B |
| LYS | C | 289 | 46.3 | 29.3 | −0.3 | 5 | B |
| LYS | O | 289 | 47.4 | 29.5 | −0.8 | 8 | B |
| ALA | N | 290 | 45.3 | 30.2 | −0.5 | 3 | B |
| ALA | CA | 290 | 45.5 | 31.4 | −1.3 | 1 | B |
| ALA | CB | 290 | 44.2 | 32.1 | −1.4 | 2 | B |
| ALA | C | 290 | 46.5 | 32.3 | −0.7 | 1 | B |
| ALA | O | 290 | 47.4 | 32.8 | −1.4 | 1 | B |
| ALA | N | 291 | 46.4 | 32.6 | 0.6 | 1 | B |
| ALA | CA | 291 | 47.3 | 33.5 | 1.2 | 1 | B |
| ALA | CB | 291 | 46.8 | 33.8 | 2.7 | 3 | B |
| ALA | C | 291 | 48.7 | 33.1 | 1.2 | 1 | B |
| ALA | O | 291 | 49.6 | 33.9 | 1.5 | 1 | B |
| SER | N | 292 | 49.0 | 31.8 | 0.9 | 1 | B |
| SER | CA | 292 | 50.3 | 31.3 | 0.9 | 1 | B |
| SER | CB | 292 | 50.5 | 30.2 | 1.9 | 1 | B |
| SER | OG | 292 | 49.3 | 29.5 | 2.0 | 4 | B |
| SER | C | 292 | 50.8 | 30.8 | −0.5 | 1 | B |
| SER | O | 292 | 51.5 | 29.8 | −0.5 | 1 | B |
| SER | N | 293 | 50.4 | 31.5 | −1.5 | 4 | B |
| SER | CA | 293 | 50.7 | 31.1 | −2.9 | 4 | B |
| SER | CB | 293 | 49.9 | 31.9 | −3.9 | 3 | B |
| SER | OG | 293 | 48.5 | 32.0 | −3.6 | 3 | B |
| SER | C | 293 | 52.1 | 31.1 | −3.3 | 4 | B |
| SER | O | 293 | 52.6 | 30.3 | −4.1 | 5 | B |
| THR | N | 294 | 52.9 | 32.1 | −2.7 | 4 | B |
| THR | CA | 294 | 54.3 | 32.2 | −3.0 | 4 | B |
| THR | CB | 294 | 55.0 | 33.0 | −1.9 | 3 | B |
| THR | OG1 | 294 | 54.2 | 34.0 | −1.4 | 3 | B |
| THR | CG2 | 294 | 56.3 | 33.6 | −2.4 | 1 | B |
| THR | C | 294 | 54.9 | 30.8 | −3.1 | 6 | B |
| THR | O | 294 | 55.6 | 30.5 | −4.0 | 4 | B |
| GLU | N | 295 | 54.6 | 30.0 | −2.1 | 7 | B |
| GLU | CA | 295 | 55.1 | 28.6 | −2.1 | 14 | B |
| GLU | CB | 295 | 55.8 | 28.3 | −0.8 | 13 | B |
| GLU | CG | 295 | 57.1 | 29.0 | −0.6 | 16 | B |
| GLU | CD | 295 | 57.8 | 28.7 | 0.7 | 18 | B |
| GLU | OE1 | 295 | 57.8 | 27.6 | 1.2 | 18 | B |
| GLU | OE2 | 295 | 58.3 | 29.7 | 1.3 | 16 | B |
| GLU | C | 295 | 54.0 | 27.6 | −2.4 | 17 | B |
| GLU | O | 295 | 52.8 | 28.0 | −2.3 | 21 | B |
| LYS | N | 296 | 54.3 | 26.4 | −2.8 | 20 | B |
| LYS | CA | 296 | 53.3 | 25.4 | −3.1 | 21 | B |
| LYS | CB | 296 | 53.4 | 25.1 | −4.6 | 28 | B |
| LYS | CG | 296 | 52.5 | 25.9 | −5.5 | 34 | B |
| LYS | CD | 296 | 51.0 | 25.5 | −5.4 | 36 | B |
| LYS | CE | 296 | 50.1 | 26.1 | −6.5 | 38 | B |
| LYS | NZ | 296 | 50.0 | 27.6 | −6.4 | 40 | B |
| LYS | C | 296 | 53.7 | 24.2 | −2.2 | 18 | B |
| LYS | O | 296 | 54.9 | 23.9 | −2.0 | 20 | B |
| PHE | N | 297 | 52.7 | 23.5 | −1.8 | 13 | B |
| PHE | CA | 297 | 52.9 | 22.3 | −1.0 | 9 | B |
| PHE | CB | 297 | 52.4 | 22.5 | 0.5 | 7 | B |
| PHE | CG | 297 | 52.5 | 23.9 | 0.9 | 9 | B |
| PHE | CD1 | 297 | 51.6 | 24.9 | 0.5 | 14 | B |
| PHE | CD2 | 297 | 53.4 | 24.3 | 1.9 | 9 | B |
| PHE | CE1 | 297 | 51.6 | 26.2 | 0.9 | 10 | B |
| PHE | CE2 | 297 | 53.5 | 25.7 | 2.3 | 5 | B |
| PHE | CZ | 297 | 52.6 | 26.6 | 1.8 | 6 | B |
| PHE | C | 297 | 52.1 | 21.1 | −1.6 | 11 | B |
| PHE | O | 297 | 51.1 | 21.4 | −2.3 | 19 | B |
| PRO | N | 298 | 52.5 | 19.9 | −1.3 | 8 | B |
| PRO | CD | 298 | 53.7 | 19.4 | −0.7 | 4 | B |
| PRO | CA | 298 | 51.7 | 18.7 | −1.9 | 11 | B |
| PRO | CB | 298 | 52.5 | 17.5 | −1.4 | 5 | B |
| PRO | CG | 298 | 53.3 | 18.1 | −0.2 | 3 | B |
| PRO | C | 298 | 50.3 | 18.7 | −1.4 | 17 | B |
| PRO | O | 298 | 50.0 | 19.1 | −0.3 | 17 | B |
| ASP | N | 299 | 49.3 | 18.3 | −2.2 | 27 | B |
| ASP | CA | 299 | 47.9 | 18.2 | −1.9 | 32 | B |
| ASP | CB | 299 | 47.1 | 17.5 | −3.1 | 39 | B |
| ASP | CG | 299 | 45.6 | 17.9 | −3.0 | 39 | B |
| ASP | OD1 | 299 | 45.3 | 19.0 | −3.2 | 42 | B |
| ASP | OD2 | 299 | 44.8 | 16.9 | −2.8 | 38 | B |
| ASP | C | 299 | 47.6 | 17.6 | −0.6 | 30 | B |
| ASP | O | 299 | 46.6 | 18.0 | 0.1 | 27 | B |
| GLY | N | 300 | 48.4 | 16.6 | −0.2 | 23 | B |
| GLY | CA | 300 | 48.2 | 15.9 | 1.1 | 17 | B |
| GLY | C | 300 | 48.5 | 16.8 | 2.3 | 13 | B |
| GLY | O | 300 | 48.0 | 16.6 | 3.4 | 13 | B |
| PHE | N | 301 | 49.3 | 17.8 | 2.0 | 9 | B |
| PHE | CA | 301 | 49.7 | 18.7 | 3.1 | 6 | B |
| PHE | CB | 301 | 50.5 | 19.9 | 2.6 | 5 | B |
| PHE | CG | 301 | 50.8 | 21.0 | 3.6 | 1 | B |
| PHE | CD1 | 301 | 51.4 | 20.7 | 4.8 | 3 | B |
| PHE | CD2 | 301 | 50.4 | 22.3 | 3.3 | 1 | B |
| PHE | CE1 | 301 | 51.6 | 21.7 | 5.8 | 1 | B |
| PHE | CE2 | 301 | 50.6 | 23.3 | 4.3 | 4 | B |
| PHE | CZ | 301 | 51.2 | 23.0 | 5.5 | 1 | B |
| PHE | C | 301 | 48.5 | 19.3 | 3.8 | 4 | B |
| PHE | O | 301 | 48.3 | 19.1 | 5.0 | 6 | B |
| TRP | N | 302 | 47.6 | 19.9 | 3.0 | 3 | B |
| TRP | CA | 302 | 46.4 | 20.6 | 3.5 | 5 | B |
| TRP | CB | 302 | 45.8 | 21.4 | 2.4 | 5 | B |
| TRP | CG | 302 | 46.6 | 22.6 | 2.0 | 4 | B |
| TRP | CD2 | 302 | 47.0 | 23.7 | 2.7 | 2 | B |
| TRP | CE2 | 302 | 47.8 | 24.5 | 1.9 | 4 | B |
| TRP | CE3 | 302 | 46.8 | 24.1 | 4.1 | 3 | B |
| TRP | CD1 | 302 | 47.2 | 22.7 | 0.7 | 4 | B |
| TRP | NE1 | 302 | 47.9 | 23.9 | 0.7 | 2 | B |
| TRP | CZ2 | 302 | 48.4 | 25.8 | 2.3 | 3 | B |
| TRP | CZ3 | 302 | 47.3 | 25.3 | 4.5 | 1 | B |
| TRP | CH2 | 302 | 48.1 | 26.1 | 3.6 | 3 | B |
| TRP | C | 302 | 45.4 | 19.6 | 4.1 | 6 | B |
| TRP | O | 302 | 44.3 | 20.0 | 4.5 | 6 | B |
| LEU | N | 303 | 45.8 | 18.3 | 4.1 | 5 | B |
| LEU | CA | 303 | 44.8 | 17.3 | 4.6 | 3 | B |
| LEU | CB | 303 | 44.4 | 16.2 | 3.6 | 1 | B |
| LEU | CG | 303 | 43.7 | 16.7 | 2.4 | 2 | B |
| LEU | CD1 | 303 | 43.2 | 15.5 | 1.7 | 9 | B |
| LEU | CD2 | 303 | 42.5 | 17.6 | 2.8 | 4 | B |
| LEU | C | 303 | 45.4 | 16.5 | 5.8 | 5 | B |
| LEU | O | 303 | 44.9 | 15.4 | 6.2 | 4 | B |
| GLY | N | 304 | 46.5 | 17.0 | 6.4 | 10 | B |
| GLY | CA | 304 | 47.2 | 16.4 | 7.5 | 11 | B |
| GLY | C | 304 | 47.8 | 15.0 | 7.2 | 10 | B |
| GLY | O | 304 | 48.3 | 14.3 | 8.1 | 11 | B |
| GLU | N | 305 | 47.9 | 14.7 | 5.9 | 10 | B |
| GLU | CA | 305 | 48.4 | 13.4 | 5.5 | 10 | B |
| GLU | CB | 305 | 47.7 | 12.9 | 4.2 | 15 | B |
| GLU | CG | 305 | 46.3 | 12.3 | 4.6 | 18 | B |
| GLU | CD | 305 | 45.4 | 12.1 | 3.3 | 27 | B |
| GLU | OE1 | 305 | 44.4 | 11.4 | 3.5 | 26 | B |
| GLU | OE2 | 305 | 45.8 | 12.6 | 2.2 | 27 | B |
| GLU | C | 305 | 49.9 | 13.5 | 5.2 | 11 | B |
| GLU | O | 305 | 50.5 | 12.4 | 5.1 | 14 | B |
| GLN | N | 306 | 50.5 | 14.7 | 5.0 | 10 | B |
| GLN | CA | 306 | 51.9 | 14.7 | 4.7 | 11 | B |
| GLN | CB | 306 | 52.2 | 14.4 | 3.3 | 14 | B |
| GLN | CG | 306 | 51.5 | 15.2 | 2.3 | 23 | B |
| GLN | CD | 306 | 51.4 | 14.6 | 0.9 | 26 | B |
| GLN | OE1 | 306 | 50.9 | 15.2 | −0.1 | 25 | B |
| GLN | NE2 | 306 | 51.9 | 13.4 | 0.8 | 23 | B |
| GLN | C | 306 | 52.5 | 16.1 | 5.1 | 9 | B |
| GLN | O | 306 | 52.0 | 17.1 | 4.7 | 9 | B |
| LEU | N | 307 | 53.6 | 16.0 | 5.9 | 9 | B |
| LEU | CA | 307 | 54.2 | 17.3 | 6.4 | 6 | B |
| LEU | CB | 307 | 55.1 | 16.9 | 7.6 | 6 | B |
| LEU | CG | 307 | 55.7 | 15.6 | 7.7 | 6 | B |
| LEU | CD1 | 307 | 56.4 | 15.2 | 6.4 | 8 | B |
| LEU | CD2 | 307 | 56.7 | 15.6 | 8.9 | 12 | B |
| LEU | C | 307 | 55.1 | 18.0 | 5.4 | 4 | B |
| LEU | O | 307 | 55.7 | 17.5 | 4.4 | 2 | B |
| VAL | N | 308 | 55.2 | 19.3 | 5.7 | 2 | B |
| VAL | CA | 308 | 56.0 | 20.3 | 4.9 | 4 | B |
| VAL | CB | 308 | 55.2 | 21.6 | 4.6 | 6 | B |
| VAL | CG1 | 308 | 56.2 | 22.7 | 4.3 | 6 | B |
| VAL | CG2 | 308 | 54.2 | 21.4 | 3.6 | 7 | B |
| VAL | C | 308 | 57.3 | 20.6 | 5.6 | 4 | B |
| VAL | O | 308 | 57.2 | 21.0 | 6.8 | 8 | B |
| CYS | N | 309 | 58.4 | 20.5 | 5.0 | 4 | B |
| CYS | CA | 309 | 59.7 | 20.8 | 5.6 | 4 | B |
| CYS | C | 309 | 60.4 | 22.0 | 5.0 | 3 | B |
| CYS | O | 309 | 60.2 | 22.3 | 3.8 | 2 | B |
| CYS | CB | 309 | 60.7 | 19.6 | 5.6 | 4 | B |
| CYS | SG | 309 | 60.0 | 18.1 | 6.3 | 9 | B |
| TRP | N | 310 | 61.2 | 22.6 | 5.8 | 1 | B |
| TRP | CA | 310 | 62.0 | 23.8 | 5.4 | 1 | B |
| TRP | CB | 310 | 61.4 | 25.1 | 5.9 | 1 | B |
| TRP | CG | 310 | 60.3 | 25.6 | 5.0 | 3 | B |
| TRP | CD2 | 310 | 58.9 | 25.7 | 5.2 | 2 | B |
| TRP | CE2 | 310 | 58.3 | 26.3 | 4.1 | 3 | B |
| TRP | CE3 | 310 | 58.1 | 25.2 | 6.3 | 1 | B |
| TRP | CD1 | 310 | 60.5 | 26.2 | 3.7 | 5 | B |
| TRP | NE1 | 310 | 59.3 | 26.6 | 3.2 | 5 | B |
| TRP | CZ2 | 310 | 56.9 | 26.4 | 4.0 | 2 | B |
| TRP | CZ3 | 310 | 56.8 | 25.4 | 6.2 | 1 | B |
| TRP | CH2 | 310 | 56.2 | 26.0 | 5.1 | 1 | B |
| TRP | C | 310 | 63.4 | 23.6 | 6.1 | 3 | B |
| TRP | O | 310 | 63.5 | 22.9 | 7.1 | 3 | B |
| GLN | N | 311 | 64.4 | 24.3 | 5.6 | 5 | B |
| GLN | CA | 311 | 65.7 | 24.3 | 6.2 | 8 | B |
| GLN | CB | 311 | 66.7 | 25.0 | 5.2 | 14 | B |
| GLN | CG | 311 | 66.4 | 24.8 | 3.7 | 24 | B |
| GLN | CD | 311 | 65.0 | 25.2 | 3.3 | 28 | B |
| GLN | OE1 | 311 | 64.6 | 26.4 | 3.2 | 30 | B |
| GLN | NE2 | 311 | 64.1 | 24.2 | 2.9 | 28 | B |
| GLN | C | 311 | 65.7 | 24.9 | 7.6 | 5 | B |
| GLN | O | 311 | 65.2 | 26.1 | 7.7 | 5 | B |
| ALA | N | 312 | 66.1 | 24.2 | 8.6 | 4 | B |
| ALA | CA | 312 | 66.1 | 24.7 | 9.9 | 1 | B |
| ALA | CB | 312 | 67.3 | 24.1 | 10.7 | 6 | B |
| ALA | C | 312 | 66.3 | 26.2 | 10.1 | 1 | B |
| ALA | O | 312 | 67.1 | 26.8 | 9.4 | 1 | B |
| GLY | N | 313 | 65.4 | 26.8 | 10.9 | 3 | B |
| GLY | CA | 313 | 65.5 | 28.2 | 11.1 | 7 | B |
| GLY | C | 313 | 65.2 | 29.1 | 9.9 | 6 | B |
| GLY | O | 313 | 65.7 | 30.2 | 9.8 | 4 | B |
| THR | N | 314 | 64.3 | 28.7 | 9.0 | 5 | B |
| THR | CA | 314 | 63.9 | 29.5 | 7.9 | 5 | B |
| THR | CB | 314 | 64.8 | 29.1 | 6.6 | 5 | B |
| THR | OG1 | 314 | 64.2 | 27.9 | 6.0 | 6 | B |
| THR | CG2 | 314 | 66.2 | 28.7 | 7.0 | 6 | B |
| THR | C | 314 | 62.5 | 29.4 | 7.5 | 2 | B |
| THR | O | 314 | 62.1 | 29.8 | 6.4 | 6 | B |
| THR | N | 315 | 61.7 | 28.8 | 8.4 | 2 | B |
| THR | CA | 315 | 60.2 | 28.7 | 8.1 | 2 | B |
| THR | CB | 315 | 59.5 | 28.0 | 9.3 | 2 | B |
| THR | OG1 | 315 | 60.3 | 26.9 | 9.7 | 1 | B |
| THR | CG2 | 315 | 58.2 | 27.6 | 8.8 | 1 | B |
| THR | C | 315 | 59.6 | 30.0 | 7.9 | 2 | B |
| THR | O | 315 | 59.6 | 30.9 | 8.8 | 4 | B |
| PRO | N | 316 | 59.1 | 30.3 | 6.7 | 1 | B |
| PRO | CD | 316 | 59.0 | 29.3 | 5.6 | 2 | B |
| PRO | CA | 316 | 58.5 | 31.6 | 6.2 | 1 | B |
| PRO | CB | 316 | 58.4 | 31.4 | 4.7 | 2 | B |
| PRO | CG | 316 | 58.0 | 29.9 | 4.6 | 1 | B |
| PRO | C | 316 | 57.1 | 31.8 | 6.9 | 2 | B |
| PRO | O | 316 | 56.1 | 31.9 | 6.2 | 4 | B |
| TRP | N | 317 | 57.1 | 32.0 | 8.2 | 2 | B |
| TRP | CA | 317 | 55.8 | 32.3 | 8.8 | 2 | B |
| TRP | CB | 317 | 56.0 | 32.6 | 10.3 | 1 | B |
| TRP | CG | 317 | 56.9 | 31.6 | 11.0 | 1 | B |
| TRP | CD2 | 317 | 56.5 | 30.3 | 11.4 | 1 | B |
| TRP | CE2 | 317 | 57.6 | 29.7 | 12.0 | 1 | B |
| TRP | CE3 | 317 | 55.3 | 29.6 | 11.3 | 2 | B |
| TRP | CD1 | 317 | 58.2 | 31.8 | 11.4 | 1 | B |
| TRP | NE1 | 317 | 58.6 | 30.6 | 11.9 | 1 | B |
| TRP | CZ2 | 317 | 57.6 | 28.4 | 12.4 | 1 | B |
| TRP | CZ3 | 317 | 55.3 | 28.3 | 11.7 | 1 | B |
| TRP | CH2 | 317 | 56.4 | 27.7 | 12.3 | 1 | B |
| TRP | C | 317 | 55.1 | 33.4 | 8.1 | 1 | B |
| TRP | O | 317 | 53.9 | 33.4 | 7.9 | 4 | B |
| ASN | N | 318 | 55.9 | 34.4 | 7.7 | 4 | B |
| ASN | CA | 318 | 55.4 | 35.6 | 7.1 | 6 | B |
| ASN | CB | 318 | 56.5 | 36.6 | 6.7 | 10 | B |
| ASN | CG | 318 | 56.8 | 36.5 | 5.2 | 8 | B |
| ASN | OD1 | 318 | 56.1 | 36.9 | 4.3 | 10 | B |
| ASN | ND2 | 318 | 58.0 | 36.0 | 4.9 | 14 | B |
| ASN | C | 318 | 54.4 | 35.3 | 6.0 | 3 | B |
| ASN | O | 318 | 53.3 | 35.9 | 5.9 | 3 | B |
| ILE | N | 319 | 54.6 | 34.3 | 5.1 | 1 | B |
| ILE | CA | 319 | 53.7 | 34.1 | 4.1 | 1 | B |
| ILE | CB | 319 | 54.3 | 33.0 | 3.0 | 1 | B |
| ILE | CG2 | 319 | 55.6 | 33.5 | 2.6 | 1 | B |
| ILE | CG1 | 319 | 54.3 | 31.7 | 3.6 | 4 | B |
| ILE | CD1 | 319 | 54.7 | 30.6 | 2.7 | 9 | B |
| ILE | C | 319 | 52.4 | 33.6 | 4.6 | 1 | B |
| ILE | O | 319 | 51.3 | 34.0 | 4.1 | 4 | B |
| PHE | N | 320 | 52.4 | 32.7 | 5.5 | 1 | B |
| PHE | CA | 320 | 51.1 | 32.1 | 6.1 | 1 | B |
| PHE | CB | 320 | 51.5 | 31.0 | 7.1 | 1 | B |
| PHE | CG | 320 | 51.9 | 29.7 | 6.4 | 1 | B |
| PHE | CD1 | 320 | 51.0 | 28.8 | 5.9 | 1 | B |
| PHE | CD2 | 320 | 53.3 | 29.5 | 6.2 | 1 | B |
| PHE | CE1 | 320 | 51.4 | 27.7 | 5.3 | 1 | B |
| PHE | CE2 | 320 | 53.7 | 28.4 | 5.6 | 1 | B |
| PHE | CZ | 320 | 52.8 | 27.4 | 5.1 | 1 | B |
| PHE | C | 320 | 50.3 | 33.2 | 6.8 | 1 | B |
| PHE | O | 320 | 50.9 | 34.1 | 7.5 | 4 | B |
| PRO | N | 321 | 49.0 | 33.2 | 6.7 | 1 | B |
| PRO | CD | 321 | 48.2 | 32.2 | 6.0 | 1 | B |
| PRO | CA | 321 | 48.1 | 34.2 | 7.3 | 1 | B |
| PRO | CB | 321 | 46.9 | 34.1 | 6.4 | 1 | B |
| PRO | CG | 321 | 46.8 | 32.7 | 6.2 | 1 | B |
| PRO | C | 321 | 47.7 | 33.9 | 8.8 | 1 | B |
| PRO | O | 321 | 47.8 | 32.7 | 9.2 | 1 | B |
| VAL | N | 322 | 47.3 | 34.9 | 9.5 | 1 | B |
| VAL | CA | 322 | 46.8 | 34.7 | 10.9 | 2 | B |
| VAL | CB | 322 | 47.0 | 35.9 | 11.8 | 3 | B |
| VAL | CG1 | 322 | 48.5 | 36.2 | 11.9 | 6 | B |
| VAL | CG2 | 322 | 46.2 | 37.0 | 11.3 | 4 | B |
| VAL | C | 322 | 45.4 | 34.3 | 10.8 | 2 | B |
| VAL | O | 322 | 44.7 | 34.8 | 9.9 | 1 | B |
| ILE | N | 323 | 44.9 | 33.5 | 11.7 | 1 | B |
| ILE | CA | 323 | 43.5 | 33.1 | 11.7 | 1 | B |
| ILE | CB | 323 | 43.3 | 31.6 | 11.8 | 1 | B |
| ILE | CG2 | 323 | 41.9 | 31.2 | 11.8 | 1 | B |
| ILE | CG1 | 323 | 44.1 | 31.0 | 10.7 | 1 | B |
| ILE | CD1 | 323 | 44.1 | 29.5 | 10.8 | 1 | B |
| ILE | C | 323 | 42.8 | 33.8 | 12.9 | 1 | B |
| ILE | O | 323 | 43.3 | 33.7 | 14.0 | 1 | B |
| SER | N | 324 | 41.7 | 34.5 | 12.7 | 1 | B |
| SER | CA | 324 | 41.0 | 35.2 | 13.8 | 1 | B |
| SER | CB | 324 | 40.9 | 36.6 | 13.6 | 1 | B |
| SER | OG | 324 | 42.2 | 37.3 | 13.6 | 1 | B |
| SER | C | 324 | 39.7 | 34.6 | 14.0 | 1 | B |
| SER | O | 324 | 39.0 | 34.3 | 13.0 | 1 | B |
| LEU | N | 325 | 39.3 | 34.3 | 15.3 | 1 | B |
| LEU | CA | 325 | 38.0 | 33.7 | 15.6 | 1 | B |
| LEU | CB | 325 | 38.2 | 32.4 | 16.3 | 1 | B |
| LEU | CG | 325 | 38.9 | 31.3 | 15.6 | 1 | B |
| LEU | CD1 | 325 | 38.9 | 30.0 | 16.4 | 1 | B |
| LEU | CD2 | 325 | 38.2 | 31.0 | 14.3 | 1 | B |
| LEU | C | 325 | 37.4 | 34.7 | 16.6 | 1 | B |
| LEU | O | 325 | 38.0 | 35.1 | 17.6 | 1 | B |
| TYR | N | 326 | 36.2 | 35.2 | 16.2 | 1 | B |
| TYR | CA | 326 | 35.4 | 36.1 | 17.1 | 1 | B |
| TYR | CB | 326 | 34.7 | 37.1 | 16.2 | 3 | B |
| TYR | CG | 326 | 35.5 | 38.2 | 15.5 | 1 | B |
| TYR | CD1 | 326 | 36.6 | 37.9 | 14.8 | 1 | B |
| TYR | CE1 | 326 | 37.4 | 38.9 | 14.1 | 2 | B |
| TYR | CD2 | 326 | 35.1 | 39.5 | 15.5 | 1 | B |
| TYR | CE2 | 326 | 35.8 | 40.5 | 14.9 | 4 | B |
| TYR | CZ | 326 | 37.0 | 40.2 | 14.2 | 1 | B |
| TYR | OH | 326 | 37.7 | 41.1 | 13.5 | 1 | B |
| TYR | C | 326 | 34.4 | 35.4 | 17.9 | 1 | B |
| TYR | O | 326 | 33.6 | 34.6 | 17.4 | 1 | B |
| LEU | N | 327 | 34.6 | 35.5 | 19.2 | 1 | B |
| LEU | CA | 327 | 33.7 | 34.9 | 20.2 | 1 | B |
| LEU | CB | 327 | 34.5 | 34.3 | 21.3 | 1 | B |
| LEU | CG | 327 | 35.7 | 33.4 | 20.9 | 1 | B |
| LEU | CD1 | 327 | 36.6 | 33.2 | 22.1 | 1 | B |
| LEU | CD2 | 327 | 35.2 | 32.1 | 20.3 | 1 | B |
| LEU | C | 327 | 32.6 | 35.8 | 20.8 | 1 | B |
| LEU | O | 327 | 33.0 | 37.0 | 21.0 | 2 | B |
| MET | N | 328 | 31.4 | 35.4 | 20.9 | 1 | B |
| MET | CA | 328 | 30.3 | 36.2 | 21.5 | 1 | B |
| MET | CB | 328 | 29.1 | 35.3 | 21.7 | 1 | B |
| MET | CG | 328 | 28.0 | 36.0 | 22.4 | 1 | B |
| MET | SD | 328 | 26.7 | 34.7 | 22.8 | 1 | B |
| MET | CE | 328 | 25.4 | 35.3 | 21.9 | 1 | B |
| MET | C | 328 | 30.8 | 36.8 | 22.8 | 1 | B |
| MET | O | 328 | 31.1 | 36.1 | 23.7 | 1 | B |
| GLY | N | 329 | 30.7 | 38.1 | 22.9 | 1 | B |
| GLY | CA | 329 | 31.1 | 38.8 | 24.1 | 6 | B |
| GLY | C | 329 | 30.1 | 38.6 | 25.3 | 7 | B |
| GLY | O | 329 | 29.1 | 38.1 | 25.1 | 9 | B |
| GLU | N | 330 | 30.5 | 39.1 | 26.4 | 6 | B |
| GLU | CA | 330 | 29.7 | 39.0 | 27.6 | 7 | B |
| GLU | CB | 330 | 30.5 | 38.9 | 28.9 | 13 | B |
| GLU | CG | 330 | 30.7 | 37.5 | 29.4 | 19 | B |
| GLU | CD | 330 | 29.4 | 36.9 | 29.9 | 27 | B |
| GLU | OE1 | 330 | 29.4 | 35.8 | 30.4 | 30 | B |
| GLU | OE2 | 330 | 28.3 | 37.5 | 29.7 | 29 | B |
| GLU | C | 330 | 28.7 | 40.2 | 27.7 | 6 | B |
| GLU | O | 330 | 27.9 | 40.2 | 28.6 | 9 | B |
| VAL | N | 331 | 28.8 | 41.2 | 26.8 | 5 | B |
| VAL | CA | 331 | 27.9 | 42.3 | 26.9 | 3 | B |
| VAL | CB | 331 | 28.6 | 43.6 | 26.7 | 1 | B |
| VAL | CG1 | 331 | 27.8 | 44.8 | 27.2 | 3 | B |
| VAL | CG2 | 331 | 30.0 | 43.5 | 27.3 | 4 | B |
| VAL | C | 331 | 26.9 | 42.1 | 25.7 | 5 | B |
| VAL | O | 331 | 27.1 | 41.3 | 24.9 | 5 | B |
| THR | N | 332 | 25.8 | 42.9 | 25.7 | 6 | B |
| THR | CA | 332 | 24.9 | 42.8 | 24.7 | 7 | B |
| THR | CB | 332 | 23.5 | 43.3 | 25.1 | 11 | B |
| THR | OG1 | 332 | 23.1 | 42.8 | 26.4 | 11 | B |
| THR | CG2 | 332 | 22.4 | 42.9 | 24.1 | 11 | B |
| THR | C | 332 | 25.4 | 43.5 | 23.4 | 4 | B |
| THR | O | 332 | 25.8 | 44.7 | 23.5 | 1 | B |
| ASN | N | 333 | 25.3 | 42.9 | 22.3 | 6 | B |
| ASN | CA | 333 | 25.8 | 43.4 | 21.0 | 7 | B |
| ASN | CB | 333 | 25.1 | 44.8 | 20.7 | 8 | B |
| ASN | CG | 333 | 23.6 | 44.5 | 20.4 | 9 | B |
| ASN | OD1 | 333 | 23.2 | 43.5 | 19.8 | 9 | B |
| ASN | ND2 | 333 | 22.8 | 45.5 | 20.6 | 5 | B |
| ASN | C | 333 | 27.3 | 43.7 | 21.1 | 6 | B |
| ASN | O | 333 | 27.7 | 44.8 | 20.6 | 9 | B |
| GLN | N | 334 | 28.1 | 42.8 | 21.6 | 7 | B |
| GLN | CA | 334 | 29.5 | 43.0 | 21.7 | 6 | B |
| GLN | CB | 334 | 29.8 | 43.8 | 23.0 | 13 | B |
| GLN | CG | 334 | 31.2 | 43.8 | 23.5 | 20 | B |
| GLN | CD | 334 | 31.6 | 45.0 | 24.3 | 27 | B |
| GLN | OE1 | 334 | 32.6 | 44.9 | 25.2 | 29 | B |
| GLN | NE2 | 334 | 31.0 | 46.1 | 24.1 | 23 | B |
| GLN | C | 334 | 30.3 | 41.7 | 21.8 | 5 | B |
| GLN | O | 334 | 29.9 | 40.7 | 22.4 | 2 | B |
| SER | N | 335 | 31.4 | 41.7 | 21.0 | 4 | B |
| SER | CA | 335 | 32.3 | 40.5 | 21.0 | 1 | B |
| SER | CB | 335 | 32.0 | 39.7 | 19.7 | 1 | B |
| SER | OG | 335 | 32.3 | 40.5 | 18.6 | 1 | B |
| SER | C | 335 | 33.7 | 40.9 | 21.0 | 1 | B |
| SER | O | 335 | 34.0 | 42.1 | 20.9 | 1 | B |
| PHE | N | 336 | 34.6 | 40.0 | 21.0 | 1 | B |
| PHE | CA | 336 | 36.0 | 40.2 | 21.0 | 1 | B |
| PHE | CB | 336 | 36.6 | 40.1 | 22.4 | 1 | B |
| PHE | CG | 336 | 36.5 | 38.7 | 23.0 | 1 | B |
| PHE | CD1 | 336 | 37.5 | 37.8 | 22.7 | 4 | B |
| PHE | CD2 | 336 | 35.5 | 38.4 | 23.8 | 1 | B |
| PHE | CE1 | 336 | 37.4 | 36.5 | 23.3 | 2 | B |
| PHE | CE2 | 336 | 35.4 | 37.1 | 24.4 | 1 | B |
| PHE | CZ | 336 | 36.4 | 36.2 | 24.1 | 1 | B |
| PHE | C | 336 | 36.6 | 39.1 | 20.1 | 1 | B |
| PHE | O | 336 | 35.9 | 38.1 | 19.8 | 1 | B |
| ARG | N | 337 | 37.8 | 39.2 | 19.7 | 1 | B |
| ARG | CA | 337 | 38.4 | 38.2 | 18.9 | 1 | B |
| ARG | CB | 337 | 38.6 | 38.8 | 17.5 | 1 | B |
| ARG | CG | 337 | 39.7 | 39.8 | 17.4 | 1 | B |
| ARG | CD | 337 | 39.8 | 40.5 | 16.1 | 1 | B |
| ARG | NE | 337 | 41.0 | 41.3 | 16.0 | 1 | B |
| ARG | CZ | 337 | 41.1 | 42.4 | 15.3 | 1 | B |
| ARG | NH1 | 337 | 40.0 | 42.8 | 14.6 | 1 | B |
| ARG | NH2 | 337 | 42.2 | 43.1 | 15.3 | 1 | B |
| ARG | C | 337 | 39.7 | 37.8 | 19.4 | 1 | B |
| ARG | O | 337 | 40.4 | 38.5 | 20.2 | 2 | B |
| ILE | N | 338 | 40.1 | 36.6 | 19.0 | 1 | B |
| ILE | CA | 338 | 41.4 | 36.0 | 19.4 | 1 | B |
| ILE | CB | 338 | 41.2 | 34.8 | 20.3 | 1 | B |
| ILE | CG2 | 338 | 40.6 | 35.2 | 21.6 | 1 | B |
| ILE | CG1 | 338 | 40.3 | 33.8 | 19.6 | 1 | B |
| ILE | CD1 | 338 | 40.2 | 32.5 | 20.4 | 1 | B |
| ILE | C | 338 | 42.1 | 35.7 | 18.1 | 1 | B |
| ILE | O | 338 | 41.4 | 35.2 | 17.2 | 1 | B |
| THR | N | 339 | 43.4 | 36.0 | 18.0 | 2 | B |
| THR | CA | 339 | 44.1 | 35.7 | 16.8 | 1 | B |
| THR | CB | 339 | 44.7 | 37.0 | 16.2 | 1 | B |
| THR | OG1 | 339 | 43.7 | 37.9 | 16.0 | 1 | B |
| THR | CG2 | 339 | 45.3 | 36.7 | 14.8 | 1 | B |
| THR | C | 339 | 45.3 | 34.7 | 17.1 | 1 | B |
| THR | O | 339 | 46.0 | 34.9 | 18.0 | 3 | B |
| ILE | N | 340 | 45.4 | 33.7 | 16.2 | 1 | B |
| ILE | CA | 340 | 46.5 | 32.7 | 16.3 | 1 | B |
| ILE | CB | 340 | 45.9 | 31.3 | 16.4 | 1 | B |
| ILE | CG2 | 340 | 44.9 | 31.2 | 17.5 | 2 | B |
| ILE | CG1 | 340 | 45.3 | 30.9 | 15.1 | 1 | B |
| ILE | CD1 | 340 | 44.7 | 29.5 | 15.1 | 2 | B |
| ILE | C | 340 | 47.3 | 32.8 | 15.1 | 1 | B |
| ILE | O | 340 | 46.8 | 33.2 | 14.0 | 1 | B |
| LEU | N | 341 | 48.6 | 32.5 | 15.2 | 2 | B |
| LEU | CA | 341 | 49.6 | 32.6 | 14.1 | 1 | B |
| LEU | CB | 341 | 50.9 | 33.2 | 14.7 | 1 | B |
| LEU | CG | 341 | 50.7 | 34.2 | 15.8 | 1 | B |
| LEU | CD1 | 341 | 52.0 | 34.5 | 16.4 | 3 | B |
| LEU | CD2 | 341 | 50.1 | 35.5 | 15.1 | 5 | B |
| LEU | C | 341 | 49.8 | 31.3 | 13.5 | 1 | B |
| LEU | O | 341 | 49.4 | 30.2 | 14.1 | 1 | B |
| PRO | N | 342 | 50.5 | 31.2 | 12.3 | 1 | B |
| PRO | CD | 342 | 51.3 | 32.3 | 11.7 | 1 | B |
| PRO | CA | 342 | 50.8 | 30.0 | 11.7 | 1 | B |
| PRO | CB | 342 | 51.6 | 30.4 | 10.5 | 2 | B |
| PRO | CG | 342 | 52.3 | 31.5 | 11.0 | 1 | B |
| PRO | C | 342 | 51.5 | 29.1 | 12.6 | 1 | B |
| PRO | O | 342 | 51.4 | 27.9 | 12.6 | 1 | B |
| GLN | N | 343 | 52.4 | 29.7 | 13.4 | 1 | B |
| GLN | CA | 343 | 53.2 | 29.0 | 14.4 | 1 | B |
| GLN | CB | 343 | 53.9 | 30.0 | 15.4 | 1 | B |
| GLN | CG | 343 | 55.1 | 30.6 | 14.8 | 1 | B |
| GLN | CD | 343 | 54.9 | 31.9 | 14.0 | 2 | B |
| GLN | OE1 | 343 | 53.8 | 32.3 | 13.7 | 5 | B |
| GLN | NE2 | 343 | 56.0 | 32.5 | 13.6 | 1 | B |
| GLN | C | 343 | 52.3 | 28.0 | 15.2 | 3 | B |
| GLN | O | 343 | 52.8 | 27.0 | 15.7 | 2 | B |
| GLN | N | 344 | 51.0 | 28.4 | 15.3 | 3 | B |
| GLN | CA | 344 | 50.1 | 27.5 | 16.1 | 3 | B |
| GLN | CB | 344 | 49.0 | 28.4 | 16.7 | 2 | B |
| GLN | CG | 344 | 49.4 | 28.9 | 18.1 | 4 | B |
| GLN | CD | 344 | 49.9 | 30.3 | 18.1 | 9 | B |
| GLN | OE1 | 344 | 50.4 | 30.7 | 19.1 | 18 | B |
| GLN | NE2 | 344 | 49.8 | 30.9 | 17.0 | 10 | B |
| GLN | C | 344 | 49.4 | 26.5 | 15.2 | 1 | B |
| GLN | O | 344 | 49.2 | 25.3 | 15.7 | 1 | B |
| TYR | N | 345 | 49.0 | 26.8 | 14.0 | 2 | B |
| TYR | CA | 345 | 48.3 | 25.8 | 13.2 | 1 | B |
| TYR | CB | 345 | 47.2 | 26.4 | 12.4 | 2 | B |
| TYR | CG | 345 | 47.6 | 27.4 | 11.3 | 3 | B |
| TYR | CD1 | 345 | 47.9 | 27.1 | 10.0 | 6 | B |
| TYR | CE1 | 345 | 48.1 | 28.1 | 9.1 | 4 | B |
| TYR | CD2 | 345 | 47.5 | 28.8 | 11.6 | 1 | B |
| TYR | CE2 | 345 | 47.7 | 29.8 | 10.7 | 1 | B |
| TYR | CZ | 345 | 48.1 | 29.4 | 9.4 | 1 | B |
| TYR | OH | 345 | 48.2 | 30.3 | 8.4 | 5 | B |
| TYR | C | 345 | 49.3 | 24.9 | 12.4 | 1 | B |
| TYR | O | 345 | 48.8 | 24.1 | 11.6 | 3 | B |
| LEU | N | 346 | 50.5 | 25.1 | 12.6 | 2 | B |
| LEU | CA | 346 | 51.5 | 24.3 | 11.9 | 3 | B |
| LEU | CB | 346 | 52.5 | 25.2 | 11.1 | 1 | B |
| LEU | CG | 346 | 51.8 | 25.8 | 9.9 | 1 | B |
| LEU | CD1 | 346 | 52.8 | 26.4 | 8.9 | 1 | B |
| LEU | CD2 | 346 | 51.0 | 24.8 | 9.1 | 1 | B |
| LEU | C | 346 | 52.3 | 23.6 | 13.1 | 5 | B |
| LEU | O | 346 | 53.1 | 24.3 | 13.8 | 6 | B |
| ARG | N | 347 | 52.0 | 22.4 | 13.3 | 4 | B |
| ARG | CA | 347 | 52.6 | 21.6 | 14.4 | 3 | B |
| ARG | CB | 347 | 51.6 | 20.6 | 14.9 | 5 | B |
| ARG | CG | 347 | 52.1 | 19.9 | 16.1 | 9 | B |
| ARG | CD | 347 | 50.9 | 19.1 | 16.8 | 14 | B |
| ARG | NE | 347 | 50.3 | 18.2 | 15.8 | 13 | B |
| ARG | CZ | 347 | 50.8 | 17.1 | 15.4 | 14 | B |
| ARG | NH1 | 347 | 52.0 | 16.8 | 15.8 | 10 | B |
| ARG | NH2 | 347 | 50.2 | 16.3 | 14.5 | 20 | B |
| ARG | C | 347 | 53.9 | 21.0 | 14.0 | 5 | B |
| ARG | O | 347 | 54.0 | 20.2 | 13.0 | 10 | B |
| PRO | N | 348 | 55.0 | 21.3 | 14.7 | 4 | B |
| PRO | CD | 348 | 55.0 | 22.2 | 15.9 | 4 | B |
| PRO | CA | 348 | 56.4 | 20.7 | 14.5 | 2 | B |
| PRO | CB | 348 | 57.2 | 21.4 | 15.6 | 3 | B |
| PRO | CG | 348 | 56.4 | 22.6 | 15.9 | 6 | B |
| PRO | C | 348 | 56.4 | 19.2 | 14.6 | 4 | B |
| PRO | O | 348 | 56.5 | 18.7 | 15.7 | 8 | B |
| VAL | N | 349 | 56.3 | 18.5 | 13.5 | 9 | B |
| VAL | CA | 349 | 56.3 | 17.0 | 13.6 | 15 | B |
| VAL | CB | 349 | 55.6 | 16.4 | 12.5 | 15 | B |
| VAL | CG1 | 349 | 54.1 | 16.0 | 13.0 | 21 | B |
| VAL | CG2 | 349 | 55.5 | 17.2 | 11.3 | 21 | B |
| VAL | C | 349 | 57.8 | 16.6 | 13.5 | 18 | B |
| VAL | O | 349 | 58.3 | 16.7 | 12.4 | 21 | B |
| GLU | N | 350 | 58.3 | 16.2 | 14.6 | 23 | B |
| GLU | CA | 350 | 59.7 | 15.8 | 14.6 | 25 | B |
| GLU | CB | 350 | 60.0 | 14.9 | 15.8 | 22 | B |
| GLU | CG | 350 | 61.2 | 13.9 | 15.6 | 25 | B |
| GLU | CD | 350 | 62.3 | 14.6 | 14.9 | 24 | B |
| GLU | OE1 | 350 | 63.0 | 13.9 | 14.1 | 19 | B |
| GLU | OE2 | 350 | 62.6 | 15.8 | 15.1 | 26 | B |
| GLU | C | 350 | 60.1 | 15.0 | 13.4 | 28 | B |
| GLU | O | 350 | 59.5 | 14.0 | 13.1 | 28 | B |
| ASP | N | 351 | 61.0 | 15.6 | 12.6 | 30 | B |
| ASP | CA | 351 | 61.4 | 15.0 | 11.3 | 30 | B |
| ASP | CB | 351 | 62.9 | 15.4 | 11.0 | 31 | B |
| ASP | CG | 351 | 63.4 | 14.7 | 9.7 | 36 | B |
| ASP | OD1 | 351 | 62.9 | 15.1 | 8.6 | 34 | B |
| ASP | OD2 | 351 | 64.2 | 13.8 | 9.8 | 34 | B |
| ASP | C | 351 | 61.3 | 13.5 | 11.3 | 30 | B |
| ASP | O | 351 | 61.4 | 12.8 | 12.4 | 28 | B |
| VAL | N | 352 | 61.1 | 12.9 | 10.1 | 29 | B |
| VAL | CA | 352 | 61.0 | 11.4 | 10.0 | 30 | B |
| VAL | CB | 352 | 61.3 | 11.0 | 8.5 | 26 | B |
| VAL | CG1 | 352 | 61.1 | 9.5 | 8.4 | 22 | B |
| VAL | CG2 | 352 | 60.3 | 11.7 | 7.6 | 22 | B |
| VAL | C | 352 | 62.0 | 10.8 | 11.0 | 33 | B |
| VAL | O | 352 | 61.6 | 10.1 | 11.9 | 35 | B |
| ALA | N | 353 | 63.3 | 11.0 | 10.8 | 35 | B |
| ALA | CA | 353 | 64.3 | 10.4 | 11.7 | 35 | B |
| ALA | CB | 353 | 65.1 | 9.3 | 11.0 | 34 | B |
| ALA | C | 353 | 65.3 | 11.4 | 12.3 | 33 | B |
| ALA | O | 353 | 66.5 | 11.2 | 12.3 | 30 | B |
| THR | N | 354 | 64.8 | 12.6 | 12.7 | 32 | B |
| THR | CA | 354 | 65.6 | 13.7 | 13.3 | 31 | B |
| THR | CB | 354 | 66.3 | 13.2 | 14.6 | 28 | B |
| THR | OG1 | 354 | 65.5 | 12.3 | 15.3 | 24 | B |
| THR | CG2 | 354 | 66.6 | 14.5 | 15.5 | 20 | B |
| THR | C | 354 | 66.6 | 14.1 | 12.2 | 32 | B |
| THR | O | 354 | 67.7 | 13.5 | 12.1 | 33 | B |
| SER | N | 355 | 66.2 | 15.2 | 11.4 | 29 | B |
| SER | CA | 355 | 67.1 | 15.7 | 10.4 | 27 | B |
| SER | CB | 355 | 66.5 | 15.5 | 9.0 | 27 | B |
| SER | OG | 355 | 65.5 | 16.4 | 8.7 | 25 | B |
| SER | C | 355 | 67.4 | 17.2 | 10.6 | 25 | B |
| SER | O | 355 | 66.8 | 17.8 | 11.5 | 19 | B |
| GLN | N | 356 | 68.2 | 17.7 | 9.8 | 26 | B |
| GLN | CA | 356 | 68.6 | 19.2 | 9.8 | 25 | B |
| GLN | CB | 356 | 69.9 | 19.4 | 9.1 | 24 | B |
| GLN | CG | 356 | 71.0 | 18.6 | 9.6 | 33 | B |
| GLN | CD | 356 | 72.3 | 19.4 | 9.8 | 34 | B |
| GLN | OE1 | 356 | 72.6 | 20.2 | 8.9 | 31 | B |
| GLN | NE2 | 356 | 73.1 | 19.2 | 10.9 | 34 | B |
| GLN | C | 356 | 67.5 | 20.0 | 9.1 | 22 | B |
| GLN | O | 356 | 67.9 | 20.9 | 8.3 | 22 | B |
| ASP | N | 357 | 66.2 | 19.7 | 9.4 | 18 | B |
| ASP | CA | 357 | 65.1 | 20.4 | 8.8 | 13 | B |
| ASP | CB | 357 | 64.6 | 19.7 | 7.5 | 14 | B |
| ASP | CG | 357 | 65.5 | 20.0 | 6.3 | 18 | B |
| ASP | OD1 | 357 | 66.7 | 19.8 | 6.5 | 22 | B |
| ASP | OD2 | 357 | 64.9 | 20.4 | 5.3 | 17 | B |
| ASP | C | 357 | 64.0 | 20.6 | 9.8 | 10 | B |
| ASP | O | 357 | 63.8 | 19.7 | 10.6 | 12 | B |
| ASP | N | 358 | 63.2 | 21.7 | 9.6 | 8 | B |
| ASP | CA | 358 | 62.1 | 21.9 | 10.5 | 8 | B |
| ASP | CB | 358 | 62.0 | 23.4 | 10.8 | 10 | B |
| ASP | CG | 358 | 63.2 | 23.9 | 11.6 | 13 | B |
| ASP | OD1 | 358 | 63.4 | 25.1 | 11.6 | 13 | B |
| ASP | OD2 | 358 | 63.9 | 23.1 | 12.1 | 13 | B |
| ASP | C | 358 | 60.9 | 21.4 | 9.6 | 7 | B |
| ASP | O | 358 | 60.8 | 21.8 | 8.5 | 9 | B |
| CYS | N | 359 | 60.1 | 20.5 | 10.2 | 10 | B |
| CYS | CA | 359 | 59.0 | 20.0 | 9.4 | 10 | B |
| CYS | C | 359 | 57.7 | 20.2 | 10.2 | 8 | B |
| CYS | O | 359 | 57.7 | 20.1 | 11.4 | 13 | B |
| CYS | CB | 359 | 59.2 | 18.5 | 9.2 | 10 | B |
| CYS | SG | 359 | 60.6 | 18.0 | 8.2 | 14 | B |
| TYR | N | 360 | 56.6 | 20.4 | 9.5 | 4 | B |
| TYR | CA | 360 | 55.4 | 20.7 | 10.2 | 1 | B |
| TYR | CB | 360 | 55.1 | 22.2 | 10.2 | 1 | B |
| TYR | CG | 360 | 56.2 | 23.0 | 10.6 | 1 | B |
| TYR | CD1 | 360 | 57.3 | 23.3 | 9.7 | 1 | B |
| TYR | CE1 | 360 | 58.3 | 24.1 | 10.2 | 1 | B |
| TYR | CD2 | 360 | 56.2 | 23.6 | 11.9 | 1 | B |
| TYR | CE2 | 360 | 57.3 | 24.3 | 12.3 | 1 | B |
| TYR | CZ | 360 | 58.3 | 24.6 | 11.4 | 2 | B |
| TYR | OH | 360 | 59.4 | 25.4 | 11.8 | 5 | B |
| TYR | C | 360 | 54.2 | 20.0 | 9.5 | 2 | B |
| TYR | O | 360 | 54.1 | 19.8 | 8.3 | 1 | B |
| LYS | N | 361 | 53.2 | 19.7 | 10.4 | 1 | B |
| LYS | CA | 361 | 51.9 | 19.1 | 9.9 | 3 | B |
| LYS | CB | 361 | 51.5 | 17.9 | 10.7 | 5 | B |
| LYS | CG | 361 | 52.1 | 16.6 | 10.1 | 14 | B |
| LYS | CD | 361 | 51.4 | 15.3 | 10.7 | 22 | B |
| LYS | CE | 361 | 52.1 | 14.1 | 10.1 | 26 | B |
| LYS | NZ | 361 | 51.7 | 12.8 | 10.7 | 25 | B |
| LYS | C | 361 | 50.9 | 20.2 | 10.0 | 1 | B |
| LYS | O | 361 | 50.8 | 20.9 | 11.0 | 1 | B |
| PHE | N | 362 | 50.0 | 20.3 | 9.0 | 2 | B |
| PHE | CA | 362 | 49.0 | 21.3 | 9.1 | 2 | B |
| PHE | CB | 362 | 48.3 | 21.4 | 7.7 | 2 | B |
| PHE | CG | 362 | 47.3 | 22.5 | 7.6 | 1 | B |
| PHE | CD1 | 362 | 47.6 | 23.8 | 7.7 | 2 | B |
| PHE | CD2 | 362 | 45.9 | 22.1 | 7.4 | 1 | B |
| PHE | CE1 | 362 | 46.7 | 24.8 | 7.6 | 1 | B |
| PHE | CE2 | 362 | 45.0 | 23.1 | 7.3 | 1 | B |
| PHE | CZ | 362 | 45.4 | 24.5 | 7.4 | 2 | B |
| PHE | C | 362 | 48.0 | 20.9 | 10.2 | 1 | B |
| PHE | O | 362 | 47.4 | 19.8 | 10.1 | 1 | B |
| ALA | N | 363 | 47.9 | 21.6 | 11.2 | 1 | B |
| ALA | CA | 363 | 47.1 | 21.3 | 12.4 | 1 | B |
| ALA | CB | 363 | 47.7 | 21.8 | 13.6 | 1 | B |
| ALA | C | 363 | 45.6 | 21.7 | 12.3 | 2 | B |
| ALA | O | 363 | 44.9 | 21.6 | 13.3 | 1 | B |
| ILE | N | 364 | 45.1 | 22.1 | 11.1 | 3 | B |
| ILE | CA | 364 | 43.7 | 22.5 | 11.0 | 1 | B |
| ILE | CB | 364 | 43.5 | 23.9 | 10.3 | 1 | B |
| ILE | CG2 | 364 | 42.1 | 24.2 | 10.2 | 1 | B |
| ILE | CG1 | 364 | 44.2 | 25.0 | 11.1 | 1 | B |
| ILE | CD1 | 364 | 44.1 | 26.3 | 10.4 | 1 | B |
| ILE | C | 364 | 43.1 | 21.4 | 10.2 | 2 | B |
| ILE | O | 364 | 43.5 | 21.2 | 9.0 | 5 | B |
| SER | N | 365 | 42.1 | 20.7 | 10.8 | 1 | B |
| SER | CA | 365 | 41.5 | 19.6 | 10.0 | 2 | B |
| SER | CB | 365 | 42.0 | 18.3 | 10.5 | 2 | B |
| SER | OG | 365 | 41.5 | 18.0 | 11.8 | 1 | B |
| SER | C | 365 | 40.0 | 19.7 | 10.1 | 1 | B |
| SER | O | 365 | 39.4 | 20.3 | 11.0 | 1 | B |
| GLN | N | 366 | 39.3 | 19.1 | 9.2 | 3 | B |
| GLN | CA | 366 | 37.8 | 19.1 | 9.1 | 3 | B |
| GLN | CB | 366 | 37.4 | 18.6 | 7.7 | 1 | B |
| GLN | CG | 366 | 38.1 | 17.4 | 7.2 | 8 | B |
| GLN | CD | 366 | 37.7 | 17.1 | 5.8 | 10 | B |
| GLN | OE1 | 366 | 36.5 | 16.8 | 5.5 | 13 | B |
| GLN | NE2 | 366 | 38.6 | 17.1 | 4.8 | 11 | B |
| GLN | C | 366 | 37.2 | 18.2 | 10.2 | 1 | B |
| GLN | O | 366 | 37.7 | 17.2 | 10.6 | 1 | B |
| SER | N | 367 | 36.0 | 18.6 | 10.6 | 3 | B |
| SER | CA | 367 | 35.2 | 17.9 | 11.6 | 8 | B |
| SER | CB | 367 | 35.2 | 18.7 | 12.9 | 9 | B |
| SER | OG | 367 | 34.2 | 18.2 | 13.7 | 16 | B |
| SER | C | 367 | 33.8 | 17.7 | 11.2 | 11 | B |
| SER | O | 367 | 33.2 | 18.5 | 10.4 | 11 | B |
| SER | N | 368 | 33.2 | 16.6 | 11.6 | 14 | B |
| SER | CA | 368 | 31.8 | 16.3 | 11.3 | 13 | B |
| SER | CB | 368 | 31.7 | 14.8 | 11.0 | 13 | B |
| SER | OG | 368 | 32.1 | 14.0 | 12.1 | 21 | B |
| SER | C | 368 | 30.9 | 16.5 | 12.6 | 10 | B |
| SER | O | 368 | 29.7 | 16.4 | 12.5 | 12 | B |
| THR | N | 369 | 31.6 | 17.0 | 13.6 | 7 | B |
| THR | CA | 369 | 30.9 | 17.3 | 14.9 | 7 | B |
| THR | CB | 369 | 31.4 | 16.4 | 16.0 | 10 | B |
| THR | OG1 | 369 | 32.8 | 16.4 | 16.1 | 11 | B |
| THR | CG2 | 369 | 30.9 | 14.9 | 15.8 | 9 | B |
| THR | C | 369 | 30.9 | 18.8 | 15.3 | 6 | B |
| THR | O | 369 | 30.8 | 19.1 | 16.5 | 1 | B |
| GLY | N | 370 | 31.0 | 19.7 | 14.4 | 5 | B |
| GLY | CA | 370 | 31.1 | 21.1 | 14.7 | 6 | B |
| GLY | C | 370 | 32.5 | 21.7 | 14.9 | 6 | B |
| GLY | O | 370 | 33.5 | 21.0 | 14.5 | 7 | B |
| THR | N | 371 | 32.7 | 22.8 | 15.5 | 2 | B |
| THR | CA | 371 | 34.0 | 23.4 | 15.8 | 1 | B |
| THR | CB | 371 | 33.9 | 24.9 | 15.7 | 1 | B |
| THR | OG1 | 371 | 33.8 | 25.4 | 14.4 | 1 | B |
| THR | CG2 | 371 | 35.1 | 25.5 | 16.3 | 1 | B |
| THR | C | 371 | 34.6 | 23.0 | 17.1 | 1 | B |
| THR | O | 371 | 33.9 | 23.1 | 18.1 | 1 | B |
| VAL | N | 372 | 35.8 | 22.6 | 17.1 | 2 | B |
| VAL | CA | 372 | 36.5 | 22.2 | 18.3 | 1 | B |
| VAL | CB | 372 | 36.9 | 20.7 | 18.2 | 1 | B |
| VAL | CG1 | 372 | 37.7 | 20.4 | 19.4 | 1 | B |
| VAL | CG2 | 372 | 35.6 | 19.9 | 18.1 | 1 | B |
| VAL | C | 372 | 37.8 | 23.0 | 18.4 | 1 | B |
| VAL | O | 372 | 38.7 | 23.0 | 17.6 | 1 | B |
| MET | N | 373 | 37.9 | 23.8 | 19.5 | 1 | B |
| MET | CA | 373 | 39.0 | 24.6 | 19.8 | 1 | B |
| MET | CB | 373 | 38.6 | 25.9 | 20.5 | 1 | B |
| MET | CG | 373 | 38.2 | 27.0 | 19.6 | 1 | B |
| MET | SD | 373 | 36.9 | 28.1 | 20.3 | 2 | B |
| MET | CE | 373 | 37.9 | 29.4 | 20.9 | 9 | B |
| MET | C | 373 | 40.1 | 23.8 | 20.5 | 1 | B |
| MET | O | 373 | 40.2 | 23.9 | 21.7 | 1 | B |
| GLY | N | 374 | 40.8 | 23.0 | 19.8 | 1 | B |
| GLY | CA | 374 | 41.8 | 22.1 | 20.3 | 1 | B |
| GLY | C | 374 | 43.1 | 22.8 | 20.8 | 1 | B |
| GLY | O | 374 | 43.2 | 24.1 | 21.0 | 2 | B |
| ALA | N | 375 | 44.1 | 22.0 | 21.1 | 1 | B |
| ALA | CA | 375 | 45.4 | 22.6 | 21.6 | 1 | B |
| ALA | CB | 375 | 46.3 | 21.5 | 21.9 | 1 | B |
| ALA | C | 375 | 46.1 | 23.6 | 20.7 | 1 | B |
| ALA | O | 375 | 46.9 | 24.3 | 21.2 | 5 | B |
| VAL | N | 376 | 45.8 | 23.7 | 19.4 | 1 | B |
| VAL | CA | 376 | 46.5 | 24.7 | 18.6 | 1 | B |
| VAL | CB | 376 | 46.3 | 24.5 | 17.1 | 1 | B |
| VAL | CG1 | 376 | 46.4 | 23.0 | 16.8 | 1 | B |
| VAL | CG2 | 376 | 45.1 | 25.1 | 16.6 | 3 | B |
| VAL | C | 376 | 45.9 | 26.0 | 19.0 | 1 | B |
| VAL | O | 376 | 46.7 | 27.0 | 19.1 | 1 | B |
| ILE | N | 377 | 44.6 | 26.1 | 19.2 | 1 | B |
| ILE | CA | 377 | 44.0 | 27.4 | 19.5 | 2 | B |
| ILE | CB | 377 | 42.4 | 27.3 | 19.4 | 2 | B |
| ILE | CG2 | 377 | 41.8 | 28.6 | 20.1 | 1 | B |
| ILE | CG1 | 377 | 42.0 | 27.2 | 18.0 | 4 | B |
| ILE | CD1 | 377 | 42.5 | 28.3 | 17.1 | 6 | B |
| ILE | C | 377 | 44.4 | 27.7 | 21.0 | 3 | B |
| ILE | O | 377 | 44.6 | 28.9 | 21.3 | 3 | B |
| MET | N | 378 | 44.5 | 26.7 | 21.8 | 3 | B |
| MET | CA | 378 | 44.8 | 26.9 | 23.2 | 4 | B |
| MET | CB | 378 | 44.4 | 25.7 | 24.0 | 1 | B |
| MET | CG | 378 | 42.9 | 25.5 | 24.2 | 2 | B |
| MET | SD | 378 | 42.5 | 24.2 | 25.3 | 1 | B |
| MET | CE | 378 | 41.9 | 25.0 | 26.6 | 4 | B |
| MET | C | 378 | 46.3 | 27.2 | 23.4 | 6 | B |
| MET | O | 378 | 46.7 | 28.0 | 24.2 | 8 | B |
| GLU | N | 379 | 47.2 | 26.5 | 22.6 | 8 | B |
| GLU | CA | 379 | 48.6 | 26.7 | 22.7 | 8 | B |
| GLU | CB | 379 | 49.4 | 25.9 | 21.7 | 10 | B |
| GLU | CG | 379 | 49.6 | 24.4 | 22.1 | 17 | B |
| GLU | CD | 379 | 50.5 | 23.7 | 21.1 | 24 | B |
| GLU | OE1 | 379 | 50.7 | 22.5 | 21.3 | 27 | B |
| GLU | OE2 | 379 | 51.0 | 24.4 | 20.2 | 25 | B |
| GLU | C | 379 | 48.9 | 28.2 | 22.5 | 7 | B |
| GLU | O | 379 | 50.1 | 28.7 | 22.6 | 11 | B |
| GLY | N | 380 | 47.9 | 29.0 | 22.1 | 5 | B |
| GLY | CA | 380 | 48.1 | 30.4 | 21.8 | 2 | B |
| GLY | C | 380 | 47.8 | 31.3 | 23.0 | 1 | B |
| GLY | O | 380 | 48.5 | 32.3 | 23.2 | 1 | B |
| PHE | N | 381 | 46.8 | 30.9 | 23.8 | 1 | B |
| PHE | CA | 381 | 46.4 | 31.8 | 24.8 | 1 | B |
| PHE | CB | 381 | 45.1 | 32.4 | 24.5 | 1 | B |
| PHE | CG | 381 | 44.9 | 32.7 | 23.1 | 4 | B |
| PHE | CD1 | 381 | 44.5 | 31.7 | 22.2 | 2 | B |
| PHE | CD2 | 381 | 45.2 | 33.9 | 22.5 | 2 | B |
| PHE | CE1 | 381 | 44.4 | 31.9 | 20.9 | 4 | B |
| PHE | CE2 | 381 | 45.1 | 34.1 | 21.2 | 1 | B |
| PHE | CZ | 381 | 44.7 | 33.1 | 20.3 | 5 | B |
| PHE | C | 381 | 46.3 | 31.2 | 26.2 | 1 | B |
| PHE | O | 381 | 46.5 | 30.0 | 26.4 | 4 | B |
| TYR | N | 382 | 46.0 | 32.0 | 27.2 | 1 | B |
| TYR | CA | 382 | 45.8 | 31.5 | 28.5 | 1 | B |
| TYR | CB | 382 | 46.3 | 32.6 | 29.5 | 1 | B |
| TYR | CG | 382 | 46.2 | 32.2 | 31.0 | 1 | B |
| TYR | CD1 | 382 | 46.8 | 31.1 | 31.5 | 1 | B |
| TYR | CE1 | 382 | 46.7 | 30.8 | 32.9 | 1 | B |
| TYR | CD2 | 382 | 45.4 | 33.0 | 31.9 | 1 | B |
| TYR | CE2 | 382 | 45.4 | 32.7 | 33.2 | 1 | B |
| TYR | CZ | 382 | 46.0 | 31.6 | 33.7 | 1 | B |
| TYR | OH | 382 | 45.9 | 31.2 | 35.0 | 1 | B |
| TYR | C | 382 | 44.3 | 31.4 | 28.6 | 1 | B |
| TYR | O | 382 | 43.6 | 32.4 | 28.4 | 6 | B |
| VAL | N | 383 | 43.8 | 30.2 | 28.9 | 1 | B |
| VAL | CA | 383 | 42.4 | 30.0 | 28.9 | 1 | B |
| VAL | CB | 383 | 42.0 | 28.7 | 28.1 | 1 | B |
| VAL | CG1 | 383 | 40.5 | 28.6 | 28.1 | 4 | B |
| VAL | CG2 | 383 | 42.6 | 28.9 | 26.7 | 1 | B |
| VAL | C | 383 | 41.9 | 29.8 | 30.3 | 1 | B |
| VAL | O | 383 | 42.5 | 29.1 | 31.1 | 1 | B |
| VAL | N | 384 | 40.9 | 30.5 | 30.7 | 1 | B |
| VAL | CA | 384 | 40.3 | 30.5 | 32.0 | 1 | B |
| VAL | CB | 384 | 40.2 | 31.8 | 32.7 | 1 | B |
| VAL | CG1 | 384 | 39.3 | 31.8 | 33.9 | 1 | B |
| VAL | CG2 | 384 | 41.5 | 32.4 | 33.0 | 1 | B |
| VAL | C | 384 | 38.9 | 29.8 | 32.0 | 1 | B |
| VAL | O | 384 | 38.0 | 30.4 | 31.3 | 3 | B |
| PHE | N | 385 | 38.7 | 28.7 | 32.7 | 1 | B |
| PHE | CA | 385 | 37.4 | 28.1 | 32.8 | 1 | B |
| PHE | CB | 385 | 37.6 | 26.6 | 32.7 | 1 | B |
| PHE | CG | 385 | 38.2 | 26.1 | 31.4 | 1 | B |
| PHE | CD1 | 385 | 37.4 | 25.6 | 30.4 | 2 | B |
| PHE | CD2 | 385 | 39.5 | 26.3 | 31.1 | 2 | B |
| PHE | CE1 | 385 | 37.9 | 25.2 | 29.1 | 1 | B |
| PHE | CE2 | 385 | 40.0 | 26.0 | 29.8 | 1 | B |
| PHE | CZ | 385 | 39.2 | 25.4 | 28.9 | 1 | B |
| PHE | C | 385 | 36.7 | 28.5 | 34.0 | 2 | B |
| PHE | O | 385 | 36.7 | 27.8 | 35.0 | 1 | B |
| ASP | N | 386 | 36.1 | 29.6 | 34.0 | 2 | B |
| ASP | CA | 386 | 35.4 | 30.2 | 35.1 | 1 | B |
| ASP | CB | 386 | 35.1 | 31.7 | 34.9 | 3 | B |
| ASP | CG | 386 | 35.0 | 32.5 | 36.1 | 2 | B |
| ASP | OD1 | 386 | 35.0 | 31.8 | 37.2 | 4 | B |
| ASP | OD2 | 386 | 34.9 | 33.7 | 36.0 | 5 | B |
| ASP | C | 386 | 34.0 | 29.5 | 35.2 | 1 | B |
| ASP | O | 386 | 33.0 | 30.1 | 35.0 | 2 | B |
| ARG | N | 387 | 34.1 | 28.2 | 35.6 | 2 | B |
| ARG | CA | 387 | 32.8 | 27.4 | 35.7 | 2 | B |
| ARG | CB | 387 | 33.1 | 26.1 | 36.3 | 1 | B |
| ARG | CG | 387 | 33.8 | 25.1 | 35.4 | 1 | B |
| ARG | CD | 387 | 34.7 | 24.2 | 36.1 | 2 | B |
| ARG | NE | 387 | 34.1 | 23.3 | 37.0 | 2 | B |
| ARG | CZ | 387 | 33.3 | 22.3 | 36.7 | 2 | B |
| ARG | NH1 | 387 | 33.0 | 22.1 | 35.5 | 3 | B |
| ARG | NH2 | 387 | 32.7 | 21.6 | 37.7 | 5 | B |
| ARG | C | 387 | 31.8 | 28.2 | 36.6 | 2 | B |
| ARG | O | 387 | 30.7 | 28.4 | 36.2 | 2 | B |
| ALA | N | 388 | 32.3 | 28.6 | 37.7 | 1 | B |
| ALA | CA | 388 | 31.4 | 29.4 | 38.7 | 4 | B |
| ALA | CB | 388 | 32.3 | 30.0 | 39.7 | 5 | B |
| ALA | C | 388 | 30.6 | 30.4 | 38.0 | 4 | B |
| ALA | O | 388 | 29.4 | 30.4 | 38.0 | 6 | B |
| ARG | N | 389 | 31.3 | 31.4 | 37.3 | 5 | B |
| ARG | CA | 389 | 30.5 | 32.4 | 36.7 | 5 | B |
| ARG | CB | 389 | 31.4 | 33.7 | 36.6 | 7 | B |
| ARG | CG | 389 | 31.8 | 34.2 | 38.0 | 12 | B |
| ARG | CD | 389 | 32.3 | 35.7 | 38.0 | 11 | B |
| ARG | NE | 389 | 31.3 | 36.6 | 37.5 | 16 | B |
| ARG | CZ | 389 | 31.5 | 37.9 | 37.3 | 20 | B |
| ARG | NH1 | 389 | 32.7 | 38.4 | 37.6 | 24 | B |
| ARG | NH2 | 389 | 30.5 | 38.7 | 36.9 | 21 | B |
| ARG | C | 389 | 30.0 | 32.1 | 35.3 | 4 | B |
| ARG | O | 389 | 29.4 | 32.9 | 34.6 | 2 | B |
| LYS | N | 390 | 30.1 | 30.8 | 34.9 | 5 | B |
| LYS | CA | 390 | 29.6 | 30.3 | 33.7 | 6 | B |
| LYS | CB | 390 | 28.1 | 30.4 | 33.7 | 9 | B |
| LYS | CG | 390 | 27.3 | 29.6 | 32.7 | 15 | B |
| LYS | CD | 390 | 25.8 | 29.8 | 32.9 | 22 | B |
| LYS | CE | 390 | 25.0 | 28.9 | 31.9 | 26 | B |
| LYS | NZ | 390 | 23.5 | 29.2 | 32.0 | 27 | B |
| LYS | C | 390 | 30.1 | 31.0 | 32.5 | 4 | B |
| LYS | O | 390 | 29.4 | 31.6 | 31.7 | 4 | B |
| ARG | N | 391 | 31.5 | 31.0 | 32.3 | 3 | B |
| ARG | CA | 391 | 32.1 | 31.7 | 31.2 | 3 | B |
| ARG | CB | 391 | 32.0 | 33.2 | 31.4 | 6 | B |
| ARG | CG | 391 | 32.8 | 33.6 | 32.7 | 8 | B |
| ARG | CD | 391 | 32.9 | 35.1 | 33.0 | 7 | B |
| ARG | NE | 391 | 33.8 | 35.4 | 34.2 | 4 | B |
| ARG | CZ | 391 | 34.1 | 36.6 | 34.6 | 1 | B |
| ARG | NH1 | 391 | 33.8 | 37.7 | 34.0 | 5 | B |
| ARG | NH2 | 391 | 34.9 | 36.7 | 35.7 | 1 | B |
| ARG | C | 391 | 33.5 | 31.3 | 30.9 | 1 | B |
| ARG | O | 391 | 34.1 | 30.7 | 31.8 | 1 | B |
| ILE | N | 392 | 34.1 | 31.7 | 29.8 | 2 | B |
| ILE | CA | 392 | 35.4 | 31.3 | 29.5 | 1 | B |
| ILE | CB | 392 | 35.5 | 30.3 | 28.4 | 1 | B |
| ILE | CG2 | 392 | 36.9 | 30.1 | 27.8 | 1 | B |
| ILE | CG1 | 392 | 35.0 | 28.9 | 28.9 | 1 | B |
| ILE | CD1 | 392 | 35.0 | 27.8 | 27.9 | 1 | B |
| ILE | C | 392 | 36.2 | 32.5 | 29.1 | 2 | B |
| ILE | O | 392 | 35.7 | 33.4 | 28.3 | 1 | B |
| GLY | N | 393 | 37.4 | 32.7 | 29.6 | 1 | B |
| GLY | CA | 393 | 38.3 | 33.8 | 29.2 | 1 | B |
| GLY | C | 393 | 39.6 | 33.4 | 28.6 | 1 | B |
| GLY | O | 393 | 40.2 | 32.5 | 28.9 | 1 | B |
| PHE | N | 394 | 39.9 | 34.2 | 27.6 | 3 | B |
| PHE | CA | 394 | 41.2 | 34.1 | 26.8 | 4 | B |
| PHE | CB | 394 | 40.9 | 33.9 | 25.3 | 1 | B |
| PHE | CG | 394 | 40.1 | 32.7 | 25.0 | 1 | B |
| PHE | CD1 | 394 | 38.7 | 32.6 | 25.4 | 1 | B |
| PHE | CD2 | 394 | 40.5 | 31.7 | 24.2 | 1 | B |
| PHE | CE1 | 394 | 37.9 | 31.6 | 25.0 | 1 | B |
| PHE | CE2 | 394 | 39.7 | 30.6 | 23.8 | 1 | B |
| PHE | CZ | 394 | 38.4 | 30.6 | 24.2 | 1 | B |
| PHE | C | 394 | 42.0 | 35.4 | 26.9 | 3 | B |
| PHE | O | 394 | 41.5 | 36.5 | 26.8 | 1 | B |
| ALA | N | 395 | 43.3 | 35.2 | 27.1 | 3 | B |
| ALA | CA | 395 | 44.3 | 36.3 | 27.2 | 1 | B |
| ALA | CB | 395 | 44.6 | 36.6 | 28.6 | 2 | B |
| ALA | C | 395 | 45.5 | 35.7 | 26.5 | 1 | B |
| ALA | O | 395 | 45.7 | 34.5 | 26.5 | 1 | B |
| VAL | N | 396 | 46.4 | 36.6 | 26.0 | 2 | B |
| VAL | CA | 396 | 47.6 | 36.2 | 25.3 | 2 | B |
| VAL | CB | 396 | 48.3 | 37.4 | 24.6 | 1 | B |
| VAL | CG1 | 396 | 49.4 | 36.8 | 23.7 | 1 | B |
| VAL | CG2 | 396 | 47.4 | 38.2 | 23.9 | 1 | B |
| VAL | C | 396 | 48.5 | 35.4 | 26.2 | 5 | B |
| VAL | O | 396 | 49.1 | 35.9 | 27.2 | 2 | B |
| SER | N | 397 | 48.8 | 34.2 | 25.8 | 7 | B |
| SER | CA | 397 | 49.6 | 33.3 | 26.5 | 10 | B |
| SER | CB | 397 | 49.6 | 31.9 | 25.9 | 14 | B |
| SER | OG | 397 | 50.4 | 31.0 | 26.6 | 21 | B |
| SER | C | 397 | 51.1 | 33.7 | 26.5 | 11 | B |
| SER | O | 397 | 51.7 | 33.9 | 25.4 | 17 | B |
| ALA | N | 398 | 51.6 | 34.0 | 27.7 | 8 | B |
| ALA | CA | 398 | 53.0 | 34.4 | 27.8 | 10 | B |
| ALA | CB | 398 | 53.2 | 34.8 | 29.3 | 11 | B |
| ALA | C | 398 | 54.0 | 33.4 | 27.4 | 11 | B |
| ALA | O | 398 | 55.2 | 33.7 | 27.4 | 12 | B |
| CYS | N | 399 | 53.5 | 32.2 | 27.0 | 8 | B |
| CYS | CA | 399 | 54.4 | 31.2 | 26.5 | 8 | B |
| CYS | C | 399 | 54.1 | 30.7 | 25.2 | 6 | B |
| CYS | O | 399 | 54.4 | 29.6 | 24.8 | 7 | B |
| CYS | CB | 399 | 54.4 | 30.0 | 27.5 | 9 | B |
| CYS | SG | 399 | 52.8 | 29.1 | 27.3 | 11 | B |
| HIS | N | 400 | 53.5 | 31.6 | 24.3 | 5 | B |
| HIS | CA | 400 | 53.2 | 31.1 | 23.0 | 9 | B |
| HIS | CB | 400 | 51.9 | 31.7 | 22.5 | 7 | B |
| HIS | CG | 400 | 52.0 | 33.0 | 21.8 | 4 | B |
| HIS | CD2 | 400 | 52.1 | 33.3 | 20.5 | 2 | B |
| HIS | ND1 | 400 | 52.1 | 34.2 | 22.5 | 6 | B |
| HIS | CE1 | 400 | 52.2 | 35.2 | 21.6 | 7 | B |
| HIS | NE2 | 400 | 52.2 | 34.7 | 20.4 | 6 | B |
| HIS | C | 400 | 54.3 | 31.4 | 22.0 | 12 | B |
| HIS | O | 400 | 55.0 | 32.4 | 22.1 | 15 | B |
| VAL | N | 401 | 54.4 | 30.5 | 21.0 | 13 | B |
| VAL | CA | 401 | 55.4 | 30.7 | 20.0 | 11 | B |
| VAL | CB | 401 | 55.6 | 29.4 | 19.2 | 8 | B |
| VAL | CG1 | 401 | 56.7 | 29.6 | 18.2 | 10 | B |
| VAL | CG2 | 401 | 55.9 | 28.3 | 20.1 | 6 | B |
| VAL | C | 401 | 54.9 | 31.8 | 19.1 | 13 | B |
| VAL | O | 401 | 53.7 | 31.9 | 18.8 | 13 | B |
| HIS | N | 402 | 55.9 | 32.7 | 18.7 | 12 | B |
| HIS | CA | 402 | 55.6 | 33.8 | 17.8 | 10 | B |
| HIS | CB | 402 | 54.9 | 34.9 | 18.5 | 8 | B |
| HIS | CG | 402 | 55.7 | 35.6 | 19.5 | 9 | B |
| HIS | CD2 | 402 | 56.2 | 36.9 | 19.6 | 11 | B |
| HIS | ND1 | 402 | 56.3 | 34.9 | 20.6 | 12 | B |
| HIS | CE1 | 402 | 57.0 | 35.8 | 21.3 | 11 | B |
| HIS | NE2 | 402 | 57.0 | 37.0 | 20.7 | 12 | B |
| HIS | C | 402 | 56.9 | 34.3 | 17.2 | 9 | B |
| HIS | O | 402 | 57.9 | 34.3 | 17.9 | 13 | B |
| ASP | N | 403 | 56.9 | 34.8 | 16.0 | 9 | B |
| ASP | CA | 403 | 58.1 | 35.3 | 15.4 | 7 | B |
| ASP | CB | 403 | 58.0 | 35.2 | 13.9 | 8 | B |
| ASP | CG | 403 | 56.9 | 36.1 | 13.3 | 9 | B |
| ASP | OD1 | 403 | 56.9 | 37.3 | 13.7 | 9 | B |
| ASP | OD2 | 403 | 56.1 | 35.5 | 12.6 | 12 | B |
| ASP | C | 403 | 58.3 | 36.7 | 15.9 | 8 | B |
| ASP | O | 403 | 57.5 | 37.2 | 16.7 | 7 | B |
| GLU | N | 404 | 59.4 | 37.3 | 15.4 | 8 | B |
| GLU | CA | 404 | 59.8 | 38.7 | 15.7 | 8 | B |
| GLU | CB | 404 | 61.1 | 39.0 | 15.0 | 6 | B |
| GLU | CG | 404 | 61.3 | 40.5 | 14.8 | 16 | B |
| GLU | CD | 404 | 62.6 | 40.8 | 14.0 | 19 | B |
| GLU | OE1 | 404 | 62.8 | 40.2 | 12.9 | 20 | B |
| GLU | OE2 | 404 | 63.3 | 41.7 | 14.4 | 23 | B |
| GLU | C | 404 | 58.7 | 39.7 | 15.4 | 7 | B |
| GLU | O | 404 | 58.5 | 40.7 | 16.1 | 8 | B |
| PHE | N | 405 | 58.0 | 39.5 | 14.2 | 6 | B |
| PHE | CA | 405 | 57.0 | 40.5 | 13.8 | 3 | B |
| PHE | CB | 405 | 57.0 | 40.5 | 12.2 | 3 | B |
| PHE | CG | 405 | 58.3 | 40.6 | 11.6 | 6 | B |
| PHE | CD1 | 405 | 59.1 | 39.5 | 11.4 | 4 | B |
| PHE | CD2 | 405 | 58.8 | 41.9 | 11.3 | 9 | B |
| PHE | CE1 | 405 | 60.4 | 39.7 | 10.9 | 6 | B |
| PHE | CE2 | 405 | 60.1 | 42.1 | 10.8 | 8 | B |
| PHE | CZ | 405 | 60.9 | 41.0 | 10.6 | 8 | B |
| PHE | C | 405 | 55.6 | 40.4 | 14.3 | 3 | B |
| PHE | O | 405 | 55.1 | 41.3 | 14.8 | 6 | B |
| ARG | N | 406 | 55.0 | 39.2 | 14.1 | 2 | B |
| ARG | CA | 406 | 53.6 | 39.0 | 14.6 | 3 | B |
| ARG | CB | 406 | 52.8 | 38.3 | 13.6 | 6 | B |
| ARG | CG | 406 | 52.7 | 39.0 | 12.2 | 11 | B |
| ARG | CD | 406 | 51.8 | 38.2 | 11.3 | 9 | B |
| ARG | NE | 406 | 52.2 | 36.9 | 11.1 | 7 | B |
| ARG | CZ | 406 | 52.0 | 36.1 | 10.0 | 4 | B |
| ARG | NH1 | 406 | 51.3 | 36.7 | 9.0 | 3 | B |
| ARG | NH2 | 406 | 52.3 | 34.9 | 10.0 | 5 | B |
| ARG | C | 406 | 53.5 | 38.4 | 16.0 | 4 | B |
| ARG | O | 406 | 54.5 | 37.9 | 16.5 | 8 | B |
| THR | N | 407 | 52.3 | 38.3 | 16.5 | 3 | B |
| THR | CA | 407 | 52.1 | 37.7 | 17.8 | 2 | B |
| THR | CB | 407 | 52.5 | 38.7 | 18.9 | 4 | B |
| THR | OG1 | 407 | 52.2 | 38.1 | 20.2 | 4 | B |
| THR | CG2 | 407 | 51.6 | 40.0 | 18.8 | 5 | B |
| THR | C | 407 | 50.6 | 37.4 | 18.0 | 1 | B |
| THR | O | 407 | 49.7 | 38.0 | 17.4 | 1 | B |
| ALA | N | 408 | 50.4 | 36.3 | 18.7 | 1 | B |
| ALA | CA | 408 | 49.0 | 35.9 | 19.0 | 1 | B |
| ALA | CB | 408 | 49.1 | 34.7 | 20.0 | 1 | B |
| ALA | C | 408 | 48.4 | 37.1 | 19.7 | 1 | B |
| ALA | O | 408 | 49.2 | 38.0 | 20.1 | 3 | B |
| ALA | N | 409 | 47.1 | 37.2 | 19.8 | 1 | B |
| ALA | CA | 409 | 46.5 | 38.3 | 20.4 | 2 | B |
| ALA | CB | 409 | 46.7 | 39.5 | 19.5 | 6 | B |
| ALA | C | 409 | 45.0 | 38.2 | 20.7 | 2 | B |
| ALA | O | 409 | 44.3 | 37.4 | 20.1 | 4 | B |
| VAL | N | 410 | 44.6 | 39.0 | 21.7 | 1 | B |
| VAL | CA | 410 | 43.2 | 39.0 | 22.1 | 1 | B |
| VAL | CB | 410 | 43.1 | 38.4 | 23.5 | 1 | B |
| VAL | CG1 | 410 | 41.6 | 38.4 | 23.9 | 5 | B |
| VAL | CG2 | 410 | 43.7 | 37.1 | 23.6 | 3 | B |
| VAL | C | 410 | 42.7 | 40.4 | 22.1 | 2 | B |
| VAL | O | 410 | 43.2 | 41.3 | 22.8 | 6 | B |
| GLU | N | 411 | 41.7 | 40.7 | 21.3 | 1 | B |
| GLU | CA | 411 | 41.2 | 42.1 | 21.2 | 2 | B |
| GLU | CB | 411 | 41.8 | 42.7 | 19.9 | 7 | B |
| GLU | CG | 411 | 43.4 | 42.9 | 20.0 | 12 | B |
| GLU | CD | 411 | 43.9 | 43.2 | 18.7 | 12 | B |
| GLU | OE1 | 411 | 45.0 | 44.0 | 18.6 | 10 | B |
| GLU | OE2 | 411 | 43.4 | 42.8 | 17.6 | 16 | B |
| GLU | C | 411 | 39.7 | 42.3 | 21.1 | 3 | B |
| GLU | O | 411 | 39.0 | 41.5 | 20.6 | 4 | B |
| GLY | N | 412 | 39.3 | 43.5 | 21.6 | 1 | B |
| GLY | CA | 412 | 37.9 | 43.8 | 21.6 | 1 | B |
| GLY | C | 412 | 37.7 | 45.3 | 21.9 | 1 | B |
| GLY | O | 412 | 38.6 | 45.9 | 22.2 | 1 | B |
| PRO | N | 413 | 36.4 | 45.8 | 21.9 | 1 | B |
| PRO | CD | 413 | 36.1 | 47.1 | 22.5 | 1 | B |
| PRO | CA | 413 | 35.2 | 45.0 | 21.7 | 2 | B |
| PRO | CB | 413 | 34.2 | 45.6 | 22.6 | 1 | B |
| PRO | CG | 413 | 34.6 | 47.1 | 22.5 | 1 | B |
| PRO | C | 413 | 34.8 | 45.2 | 20.2 | 4 | B |
| PRO | O | 413 | 35.2 | 46.2 | 19.6 | 7 | B |
| PHE | N | 414 | 34.0 | 44.4 | 19.7 | 3 | B |
| PHE | CA | 414 | 33.5 | 44.5 | 18.3 | 3 | B |
| PHE | CB | 414 | 34.1 | 43.4 | 17.4 | 5 | B |
| PHE | CG | 414 | 35.6 | 43.4 | 17.4 | 5 | B |
| PHE | CD1 | 414 | 36.3 | 42.6 | 18.2 | 11 | B |
| PHE | CD2 | 414 | 36.2 | 44.3 | 16.6 | 9 | B |
| PHE | CE1 | 414 | 37.7 | 42.6 | 18.3 | 13 | B |
| PHE | CE2 | 414 | 37.6 | 44.4 | 16.6 | 13 | B |
| PHE | CZ | 414 | 38.4 | 43.5 | 17.5 | 13 | B |
| PHE | C | 414 | 32.0 | 44.5 | 18.2 | 6 | B |
| PHE | O | 414 | 31.4 | 43.5 | 18.8 | 10 | B |
| VAL | N | 415 | 31.4 | 45.5 | 17.6 | 6 | B |
| VAL | CA | 415 | 30.0 | 45.5 | 17.5 | 8 | B |
| VAL | CB | 415 | 29.4 | 46.8 | 16.9 | 6 | B |
| VAL | CG1 | 415 | 28.0 | 47.0 | 17.2 | 7 | B |
| VAL | CG2 | 415 | 30.2 | 48.0 | 17.3 | 9 | B |
| VAL | C | 415 | 29.5 | 44.3 | 16.6 | 7 | B |
| VAL | O | 415 | 30.0 | 44.2 | 15.5 | 7 | B |
| THR | N | 416 | 28.6 | 43.5 | 17.2 | 8 | B |
| THR | CA | 416 | 28.1 | 42.4 | 16.4 | 7 | B |
| THR | CB | 416 | 28.9 | 41.1 | 16.7 | 1 | B |
| THR | OG1 | 416 | 30.3 | 41.4 | 16.6 | 1 | B |
| THR | CG2 | 416 | 28.5 | 40.1 | 15.6 | 2 | B |
| THR | C | 416 | 26.7 | 42.2 | 17.0 | 10 | B |
| THR | O | 416 | 26.5 | 42.1 | 18.2 | 13 | B |
| LEU | N | 417 | 25.7 | 42.2 | 16.1 | 9 | B |
| LEU | CA | 417 | 24.3 | 42.1 | 16.4 | 8 | B |
| LEU | CB | 417 | 23.5 | 43.1 | 15.5 | 6 | B |
| LEU | CG | 417 | 24.0 | 44.5 | 15.5 | 8 | B |
| LEU | CD1 | 417 | 23.5 | 45.2 | 14.2 | 5 | B |
| LEU | CD2 | 417 | 23.5 | 45.3 | 16.7 | 8 | B |
| LEU | C | 417 | 23.8 | 40.7 | 16.3 | 8 | B |
| LEU | O | 417 | 24.3 | 40.0 | 15.4 | 6 | B |
| ASP | N | 418 | 22.8 | 40.4 | 17.1 | 13 | B |
| ASP | CA | 418 | 22.2 | 39.0 | 17.0 | 17 | B |
| ASP | CB | 418 | 21.4 | 38.9 | 15.7 | 21 | B |
| ASP | CG | 418 | 20.3 | 40.0 | 15.6 | 27 | B |
| ASP | OD1 | 418 | 20.0 | 40.3 | 14.4 | 25 | B |
| ASP | OD2 | 418 | 19.8 | 40.5 | 16.6 | 27 | B |
| ASP | C | 418 | 23.2 | 37.9 | 17.0 | 17 | B |
| ASP | O | 418 | 23.2 | 37.0 | 16.1 | 19 | B |
| MET | N | 419 | 24.1 | 37.9 | 18.0 | 16 | B |
| MET | CA | 419 | 25.1 | 36.9 | 18.1 | 14 | B |
| MET | CB | 419 | 26.1 | 37.2 | 19.2 | 9 | B |
| MET | CG | 419 | 26.9 | 38.5 | 18.8 | 10 | B |
| MET | SD | 419 | 28.1 | 38.9 | 20.0 | 5 | B |
| MET | CE | 419 | 27.2 | 39.9 | 21.1 | 14 | B |
| MET | C | 419 | 24.6 | 35.4 | 18.4 | 15 | B |
| MET | O | 419 | 25.2 | 34.5 | 17.9 | 17 | B |
| GLU | N | 420 | 23.5 | 35.3 | 19.1 | 19 | B |
| GLU | CA | 420 | 23.0 | 34.0 | 19.4 | 21 | B |
| GLU | CB | 420 | 22.4 | 34.0 | 20.8 | 24 | B |
| GLU | CG | 420 | 22.4 | 32.6 | 21.5 | 26 | B |
| GLU | CD | 420 | 22.8 | 32.7 | 23.0 | 27 | B |
| GLU | OE1 | 420 | 22.4 | 33.7 | 23.7 | 27 | B |
| GLU | OE2 | 420 | 23.6 | 31.8 | 23.5 | 23 | B |
| GLU | C | 420 | 21.9 | 33.6 | 18.4 | 22 | B |
| GLU | O | 420 | 21.9 | 34.1 | 17.3 | 22 | B |
| GLU | OXT | 420 | 21.0 | 32.8 | 18.8 | 21 | B |

The structural coordinates for the above-described BACE crystal are set forth below.
“Res.” refers to the amino acid whose atomic coordinates have been determined.
“Atom” refers to the atom, of the corresponding residue, whose coordinates have been determined.
“#” refers to the amino acid number of the corresponding residue.
“X”, “Y” and Z” refer to the crystallographically determined atomic position determined for each atom.
“B” refers to a thermal factor that measures movement of the atom around its atomic center.
“C” refers to the molecule to which the corresponding residue belongs.

### Example 8

Crystallization of SF-9 Derived β-Secretase (Pyramidal).

ProBACE (SEQ ID NO:2) in 20 mM Hepes, pH 7.5, 150 mM NaCl was concentrated by centrifugal filtration to 0.18 to 0.36 mM (10-20 mg / ml) followed by ultra-centrifuigation prior to crystallization. Vapor diffusion crystallization experiments were conducted using the hanging drop method. Crystals were grown from a droplet containing 1 μl of protein and 1 μl of the reservoir solution (0.1 M sodium citrate (Fluka BioChemika, Germany), pH 4.0, 10-30 % polyethylene glycol 6000 (Fluka BioChemika, Germany), and 0.2-1.0 M lithium chloride (Fluka BioChemika, Germany). At pH 4.0, the proBACE was autoprocessed to BACE (SEQ ID NO: 4) within the droplet. Crystallization plates were incubated at 4° C., which grew pyramidal crystals (0.05×0.05×0.05 mm) over 40-120 days (based on mass spectral and immunoblot data of redissolved crystals consistent with BACE SEQ ID NO: 4)

BACE

SEQ ID NO: 4

(Residues 27-429 of SEQ ID NO: 2)
Molecular weight 47.8 KDa
ALTERNATIVE PROCESSING (CAT DOMAIN, 403 AA)
DEEPEEPGRR GSFVEMVDNL RGKSGQGYYV EMTVGSPPQT
LNILVDTGSS NFAVGAAPHP FLHRYYQRQL SSTYRDLRKG
VYVPYTQGKW EGELGTDLVS IPHGPNVTVR ANIAAITESD
KFFINGSNWE GILGLAYAEI ARPDDSLEPF FDSLVKQTHV
PNLFSLQLCG AGFPLNQSEV LASVGGSMII GGIDHSLYTG
SLWYTPIRRE WYYEVIIVRV EINGQDLKMD CKEYNYDKSI
VDSGTTNLRL PKKVFEAAVK SIKAASSTEK FPDGFWLGEQ
LVCWQAGTTP WNIFPVISLY LMGEVTNQSF RITILPQQYL
RPVEDVATSQ DDCYKFAISQ SSTGTVMGAV IMEGFYVVFD
RARKRIGFAV SACHVHDEFR TAAVEGPFVT LDMEDCGYNI
PQT

### Example 9

BACE/OM-99-2 Crystalline Complex

Auto processing step. ProBACE (SEQ ID NO:2) in 100 mM sodium acetate, pH 4.0, was concentrated by centrifugal filtration to 0.09 to 0.18 mM (5-10 mg / ml). The concentrate was incubated at 22° C. for 18 hours. The resulting mixture was mostly BACE (SEQ ID NO: 5) as analyzed by N-terminal, mass spectral and immunoblot analysis.

Alternatively, ProBACE may be autoprocessed as follows. ProBACE was concentrated to 10 mg/ml, mixed with 1 volume of 100 mM NaOAc, pH 4.0, and was dialyzed against the same buffer with mild stirring for 9 -16 h at room temperature. At the end of the reaction, the mixture was adjusted to pH 8.0 by adding 0.7 volume of 1 M Hepes buffer, pH 8.0, and the solution was immediately applied to Superdex 200 (High Load, 26/60, Amersham Pharmacia, Piscataway, N.J.), equilibrated in 20 mM Hepes, pH 7.5, 150 mM NaCl. The fractions containing BACE of >95% purity were concentrated, flash-frozen in liquid N₂, and stored at −80° C.

BACE

SEQ ID NO: 5

(Residues 22-429 OF SEQ ID NO: 2)
LPRETDEEPE EPGRRGSFVE MVDNLRGKSG QGYYVEMTVG
SPPQTLNILV DTGSSNFAVG AAPHPFLHRY YQRQLSSTYR
DLRKGVYVPY TQGKWEGELG TDLVSIPHGP NVTVRANIAA
ITESDKFFIN GSNWEGILGL AYAEIARPDD SLEPFFDSLV
KQTHVPNLFS LQLCGAGFPL NQSEVLASVG GSMIIGGIDH
SLYTGSLWYT PIRREWYYEV IIVRVEINGQ DLKMDCKEYN
YDKSIVDSGT TNLRLPKKVF EAAVKSIKAA SSTEKFPDGF
WLGEQLVCWQ AGTTPWNIFP VISLYLMGEV TNQSFRITIL
PQQYLRPVED VATSQDDCYK FAISQSSTGT VMGAVIMEGF
YVVFDRARKR IGFAVSACHV HDEFRTAAVE GPFVTLDMED
CGYNIPQT

Processing. The processed BACE (SEQ ID NO: 5) was complexed with OM-99-2 (Hong, et al., (2000) Science 290: 150-153; Bachem Bioscience Inc.; King of Prussia, Pa.), an inhibitor of BACE at a 1:5 molar ratio.

It should be noted that OM-99-2 is a transition state mimetic that is also characterized by the structure EVN{(2R,4S,5S)-5-amino-4-hydroxy-2,7-dimethyl-octsnoyl}AEF. The chemical structure of OM-99-2 is shown below:

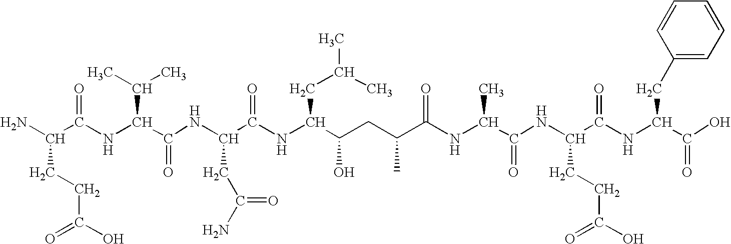

The complex was then incubated on ice for 5 minutes in 20 mM Hepes, pH 7.5, 150 mM NaCl was concentrated by centrifugal filtration to 0.18 to 0.36 mM (10-20 mg /ml) followed by ultra-centrifugation prior to crystallization. Vapor diffusion crystallization experiments were conducted using the hanging drop method. Crystals were grown from a droplet containing 1 μl of protein and 1 μl of the reservoir solution (0.1 M TRIS (Fluka BioChemika, Germany), pH 8.0, 10-30 % polyethylene glycol 3000 (Fluka BioChemika, Germany), and 0.1-1.0 M calcium acetate (Fluka BioChemika, Germany). Crystallization plates were incubated at 4° C., which grew rectangular rods (0.04×0.4 mm) over 3-5 days.

Crystals were removed from the crystallization droplet by addition of 20% glycerol, which permitted freezing under both cold nitrogen stream and liquid propane. Diffraction data of β-secretase crystal was determined from a Rigaku R-Axis IV image plate detector mounted on a Rigaku RU-HR rotating anode generator Cu radiation 1.54 Å operating at 100 mA and 50 kV.

Data Collection Statistics:

| | |
|---|---|
| Resolution | 50.0-2.25 Å |
| No. of collected reflections | 227292 |
| No. of unique reflections (F >= 0) | 44920 |
| R-sym | 6.7% |
| Percent of theoretical (I/s >= 1) | 97.1% |
| Unit Cell | a = 54.96 Å, b = 99.42 Å, |
| | c = 94.83 Å, α = 90.0 |
| | β = 107.0 γ = 90.0° |
| Space Group | P2₁2₁2₁ |
| Asymmetric unit | 2 molecules |

TABLE 3
Structural coordinates for BACE/OM-99-2 Complex.

| Res. | At. | Ch. | # | X | Y | Z | B |
|---|---|---|---|---|---|---|---|
| SER | CB | A | 38 | −3.8 | −38.4 | 34.5 | 72 |
| SER | OG | A | 38 | −4.3 | −39.2 | 35.6 | 74 |
| SER | C | A | 38 | −2.0 | −38.0 | 32.9 | 68 |
| SER | O | A | 38 | −2.5 | −38.2 | 31.8 | 69 |
| SER | N | A | 38 | −1.4 | −38.6 | 35.2 | 69 |
| SER | CA | A | 38 | −2.4 | −38.8 | 34.1 | 70 |
| PHE | N | A | 39 | −1.0 | −37.1 | 33.1 | 63 |
| PHE | CA | A | 39 | −0.5 | −36.3 | 32.0 | 58 |
| PHE | CB | A | 39 | −1.0 | −34.8 | 32.3 | 55 |
| PHE | CG | A | 39 | −2.4 | −34.7 | 32.4 | 54 |
| PHE | CD1 | A | 39 | −3.1 | −35.0 | 33.6 | 53 |
| PHE | CD2 | A | 39 | −3.2 | −34.2 | 31.4 | 54 |
| PHE | CE1 | A | 39 | −4.5 | −34.9 | 33.8 | 53 |
| PHE | CE2 | A | 39 | −4.6 | −34.1 | 31.5 | 54 |
| PHE | CZ | A | 39 | −5.2 | −34.4 | 32.7 | 55 |
| PHE | C | A | 39 | 1.0 | −36.4 | 31.9 | 57 |
| PHE | O | A | 39 | 1.7 | −35.4 | 31.6 | 57 |
| VAL | N | A | 40 | 1.5 | −37.6 | 32.1 | 56 |
| VAL | CA | A | 40 | 3.0 | −37.8 | 32.0 | 55 |
| VAL | CB | A | 40 | 3.2 | −39.4 | 32.2 | 56 |
| VAL | CG1 | A | 40 | 4.7 | −39.7 | 32.1 | 57 |
| VAL | CG2 | A | 40 | 2.6 | −39.9 | 33.4 | 57 |
| VAL | C | A | 40 | 3.6 | −37.3 | 30.8 | 56 |
| VAL | O | A | 40 | 4.9 | −37.1 | 30.8 | 55 |
| GLU | N | A | 41 | 2.9 | −37.0 | 29.7 | 57 |
| GLU | CA | A | 41 | 3.5 | −36.5 | 28.5 | 55 |
| GLU | CB | A | 41 | 2.6 | −36.8 | 27.2 | 61 |
| GLU | CG | A | 41 | 2.2 | −38.2 | 27.0 | 67 |
| GLU | CD | A | 41 | 1.3 | −38.8 | 28.1 | 70 |
| GLU | OE1 | A | 41 | 0.8 | −38.1 | 28.9 | 70 |
| GLU | OE2 | A | 41 | 1.3 | −40.1 | 28.2 | 72 |
| GLU | C | A | 41 | 3.8 | −35.0 | 28.6 | 50 |
| GLU | O | A | 41 | 4.5 | −34.5 | 27.8 | 48 |
| MET | N | A | 42 | 3.1 | −34.4 | 29.5 | 44 |
| MET | CA | A | 42 | 3.3 | −32.9 | 29.7 | 40 |
| MET | CB | A | 42 | 1.9 | −32.2 | 29.8 | 40 |
| MET | CG | A | 42 | 1.1 | −32.4 | 28.5 | 39 |
| MET | SD | A | 42 | −0.5 | −31.6 | 28.6 | 43 |
| MET | CE | A | 42 | −1.5 | −32.9 | 29.0 | 44 |
| MET | C | A | 42 | 4.2 | −32.6 | 30.9 | 37 |
| MET | O | A | 42 | 4.5 | −31.4 | 31.1 | 38 |
| VAL | N | A | 43 | 4.5 | −33.5 | 31.7 | 34 |
| VAL | CA | A | 43 | 5.4 | −33.3 | 32.9 | 33 |
| VAL | CB | A | 43 | 5.4 | −34.5 | 33.8 | 33 |
| VAL | CG1 | A | 43 | 6.4 | −34.1 | 35.0 | 33 |
| VAL | CG2 | A | 43 | 4.0 | −34.8 | 34.4 | 31 |
| VAL | C | A | 43 | 6.8 | −32.9 | 32.4 | 31 |
| VAL | O | A | 43 | 7.4 | −33.5 | 31.6 | 32 |
| ASP | N | A | 44 | 7.2 | −31.8 | 33.0 | 29 |
| ASP | CA | A | 44 | 8.6 | −31.3 | 32.7 | 35 |
| ASP | CB | A | 44 | 9.6 | −32.3 | 33.1 | 42 |
| ASP | CG | A | 44 | 11.0 | −31.8 | 32.9 | 50 |
| ASP | OD1 | A | 44 | 11.7 | −31.5 | 33.9 | 56 |
| ASP | OD2 | A | 44 | 11.5 | −31.8 | 31.8 | 54 |
| ASP | C | A | 44 | 8.7 | −30.7 | 31.3 | 32 |
| ASP | O | A | 44 | 9.7 | −30.8 | 30.7 | 32 |
| ASN | N | A | 45 | 7.6 | −30.2 | 30.7 | 27 |
| ASN | CA | A | 45 | 7.6 | −29.6 | 29.4 | 25 |
| ASN | CB | A | 45 | 6.3 | −29.8 | 28.7 | 27 |
| ASN | CG | A | 45 | 5.2 | −29.1 | 29.4 | 26 |
| ASN | OD1 | A | 45 | 5.3 | −28.5 | 30.4 | 26 |
| ASN | ND2 | A | 45 | 4.0 | −29.2 | 28.8 | 22 |
| ASN | C | A | 45 | 8.1 | −28.2 | 29.4 | 27 |
| ASN | O | A | 45 | 8.1 | −27.5 | 28.3 | 28 |
| LEU | N | A | 46 | 8.3 | −27.6 | 30.6 | 23 |
| LEU | CA | A | 46 | 8.8 | −26.2 | 30.7 | 24 |
| LEU | CB | A | 46 | 7.9 | −25.4 | 31.7 | 23 |
| LEU | CG | A | 46 | 6.4 | −25.4 | 31.4 | 22 |
| LEU | CD1 | A | 46 | 5.7 | −24.6 | 32.4 | 21 |
| LEU | CD2 | A | 46 | 6.2 | −24.7 | 30.0 | 21 |
| LEU | C | A | 46 | 10.2 | −26.1 | 31.0 | 29 |
| LEU | O | A | 46 | 10.8 | −26.8 | 31.9 | 28 |
| ARG | N | A | 47 | 10.9 | −25.1 | 30.4 | 29 |
| ARG | CA | A | 47 | 12.3 | −24.8 | 30.7 | 33 |
| ARG | CB | A | 47 | 13.2 | −25.3 | 29.6 | 34 |
| ARG | CG | A | 47 | 13.2 | −26.9 | 29.5 | 38 |
| ARG | CD | A | 47 | 14.2 | −27.3 | 28.5 | 43 |
| ARG | NE | A | 47 | 13.9 | −26.7 | 27.1 | 45 |
| ARG | CZ | A | 47 | 14.8 | −26.6 | 26.2 | 47 |
| ARG | NH1 | A | 47 | 16.1 | −26.9 | 26.4 | 48 |
| ARG | NH2 | A | 47 | 14.5 | −26.1 | 25.0 | 47 |
| ARG | C | A | 47 | 12.4 | −23.3 | 30.8 | 35 |
| ARG | O | A | 47 | 11.5 | −22.5 | 30.5 | 31 |
| GLY | N | A | 48 | 13.6 | −22.8 | 31.2 | 36 |
| GLY | CA | A | 48 | 13.8 | −21.4 | 31.3 | 45 |
| GLY | C | A | 48 | 15.1 | −21.0 | 32.1 | 50 |
| GLY | O | A | 48 | 15.7 | −21.9 | 32.7 | 53 |
| LYS | N | A | 49 | 15.4 | −19.8 | 32.0 | 50 |
| LYS | CA | A | 49 | 16.6 | −19.2 | 32.7 | 49 |
| LYS | CB | A | 49 | 17.6 | −18.6 | 31.8 | 52 |
| LYS | CG | A | 49 | 18.2 | −19.6 | 30.8 | 58 |
| LYS | CD | A | 49 | 19.2 | −18.9 | 29.9 | 62 |
| LYS | CE | A | 49 | 19.8 | −19.9 | 28.9 | 65 |
| LYS | NZ | A | 49 | 20.8 | −19.2 | 28.0 | 66 |
| LYS | C | A | 49 | 16.1 | −18.2 | 33.7 | 47 |
| LYS | O | A | 49 | 15.2 | −17.4 | 33.4 | 49 |
| SER | N | A | 50 | 16.6 | −18.4 | 35.0 | 44 |
| SER | CA | A | 50 | 16.1 | −17.5 | 36.1 | 42 |
| SER | CB | A | 50 | 17.1 | −17.6 | 37.3 | 39 |
| SER | OG | A | 50 | 16.6 | −16.7 | 38.3 | 40 |
| SER | C | A | 50 | 15.9 | −16.0 | 35.7 | 40 |
| SER | O | A | 50 | 16.8 | −15.4 | 35.1 | 42 |
| GLY | N | A | 51 | 14.8 | −15.5 | 35.9 | 37 |
| GLY | CA | A | 51 | 14.5 | −14.1 | 35.6 | 31 |
| GLY | C | A | 51 | 14.3 | −13.8 | 34.1 | 32 |
| GLY | O | A | 51 | 14.4 | −12.7 | 33.7 | 29 |
| GLN | N | A | 52 | 14.0 | −14.9 | 33.4 | 29 |
| GLN | CA | A | 52 | 13.8 | −14.8 | 32.0 | 31 |
| GLN | CB | A | 52 | 15.0 | −15.2 | 31.2 | 33 |
| GLN | CG | A | 52 | 16.3 | −14.3 | 31.5 | 39 |
| GLN | CD | A | 52 | 17.4 | −14.8 | 30.7 | 41 |
| GLN | OE1 | A | 52 | 17.4 | −15.8 | 30.0 | 46 |
| GLN | NE2 | A | 52 | 18.5 | −14.0 | 30.7 | 42 |
| GLN | C | A | 52 | 12.5 | −15.5 | 31.5 | 29 |
| GLN | O | A | 52 | 12.4 | −15.7 | 30.2 | 29 |
| GLY | N | A | 53 | 11.7 | −15.9 | 32.4 | 26 |
| GLY | CA | A | 53 | 10.5 | −16.6 | 32.0 | 22 |
| GLY | C | A | 53 | 10.6 | −18.1 | 31.7 | 29 |
| GLY | O | A | 53 | 11.7 | −18.6 | 31.6 | 30 |
| TYR | N | A | 54 | 9.4 | −18.8 | 31.7 | 29 |
| TYR | CA | A | 54 | 9.3 | −20.2 | 31.4 | 29 |
| TYR | CB | A | 54 | 8.5 | −20.9 | 32.4 | 26 |
| TYR | CG | A | 54 | 9.0 | −20.9 | 33.8 | 26 |
| TYR | CD1 | A | 54 | 9.0 | −19.8 | 34.6 | 27 |
| TYR | CE1 | A | 54 | 9.5 | −19.9 | 36.0 | 29 |
| TYR | CD2 | A | 54 | 9.6 | −22.1 | 34.3 | 27 |
| TYR | CE2 | A | 54 | 10.1 | −22.2 | 35.6 | 29 |
| TYR | CZ | A | 54 | 10.0 | −21.0 | 36.4 | 32 |
| TYR | OH | A | 54 | 10.5 | −21.1 | 37.7 | 32 |
| TYR | C | A | 54 | 8.7 | −20.4 | 30.0 | 31 |
| TYR | O | A | 54 | 7.8 | −19.7 | 29.6 | 30 |
| TYR | N | A | 55 | 9.4 | −21.2 | 29.2 | 28 |
| TYR | CA | A | 55 | 8.9 | −21.5 | 27.8 | 32 |
| TYR | CB | A | 55 | 9.9 | −20.9 | 26.8 | 33 |
| TYR | CG | A | 55 | 11.3 | −21.5 | 26.9 | 33 |
| TYR | CD1 | A | 55 | 11.6 | −22.7 | 26.2 | 30 |
| TYR | CE1 | A | 55 | 12.8 | −23.3 | 26.3 | 30 |
| TYR | CD2 | A | 55 | 12.3 | −20.9 | 27.6 | 33 |
| TYR | CE2 | A | 55 | 13.6 | −21.5 | 27.6 | 35 |
| TYR | CZ | A | 55 | 13.8 | −22.7 | 27.0 | 33 |
| TYR | OH | A | 55 | 15.1 | −23.3 | 27.1 | 35 |
| TYR | C | A | 55 | 8.6 | −22.9 | 27.5 | 34 |
| TYR | O | A | 55 | 9.2 | −23.9 | 28.0 | 28 |
| VAL | N | A | 56 | 7.6 | −23.1 | 26.6 | 35 |
| VAL | CA | A | 56 | 7.2 | −24.4 | 26.2 | 33 |
| VAL | CB | A | 56 | 5.7 | −24.7 | 26.4 | 32 |
| VAL | CG1 | A | 56 | 4.8 | −23.7 | 25.6 | 24 |
| VAL | CG2 | A | 56 | 5.3 | −26.1 | 26.1 | 33 |
| VAL | C | A | 56 | 7.5 | −24.6 | 24.7 | 33 |
| VAL | O | A | 56 | 7.3 | −23.6 | 23.9 | 30 |
| GLU | N | A | 57 | 7.9 | −25.7 | 24.2 | 31 |
| GLU | CA | A | 57 | 8.2 | −25.9 | 22.8 | 32 |
| GLU | CB | A | 57 | 9.0 | −27.2 | 22.6 | 33 |
| GLU | CG | A | 57 | 9.4 | −27.4 | 21.1 | 35 |
| GLU | CD | A | 57 | 10.2 | −28.6 | 20.9 | 39 |
| GLU | OE1 | A | 57 | 11.4 | −28.6 | 21.1 | 39 |
| GLU | OE2 | A | 57 | 9.6 | −29.7 | 20.5 | 40 |
| GLU | C | A | 57 | 6.9 | −26.0 | 22.0 | 30 |
| GLU | O | A | 57 | 5.9 | −26.6 | 22.5 | 33 |
| MET | N | A | 58 | 6.9 | −25.3 | 20.9 | 30 |
| MET | CA | A | 58 | 5.7 | −25.3 | 20.0 | 31 |
| MET | CB | A | 58 | 4.8 | −24.1 | 20.3 | 31 |
| MET | CG | A | 58 | 4.2 | −24.0 | 21.6 | 30 |
| MET | SD | A | 58 | 3.1 | −22.5 | 21.8 | 31 |
| MET | CE | A | 58 | 1.5 | −23.1 | 21.3 | 31 |
| MET | C | A | 58 | 6.2 | −25.4 | 18.6 | 29 |
| MET | O | A | 58 | 7.3 | −25.2 | 18.3 | 27 |
| THR | N | A | 59 | 5.2 | −25.7 | 17.7 | 32 |
| THR | CA | A | 59 | 5.5 | −25.8 | 16.3 | 35 |
| THR | CB | A | 59 | 5.6 | −27.2 | 15.7 | 34 |
| THR | OG1 | A | 59 | 4.3 | −27.9 | 15.9 | 40 |
| THR | OG2 | A | 59 | 6.7 | −28.0 | 16.4 | 31 |
| THR | C | A | 59 | 4.4 | −25.0 | 15.5 | 36 |
| THR | O | A | 59 | 3.2 | −25.1 | 15.7 | 39 |
| VAL | N | A | 60 | 4.9 | −24.1 | 14.6 | 35 |
| VAL | CA | A | 60 | 4.0 | −23.3 | 13.7 | 39 |
| VAL | CB | A | 60 | 4.1 | −21.8 | 14.1 | 38 |
| VAL | CG1 | A | 60 | 3.7 | −21.6 | 15.5 | 40 |
| VAL | CG2 | A | 60 | 5.4 | −21.3 | 13.7 | 40 |
| VAL | C | A | 60 | 4.3 | −23.6 | 12.3 | 41 |
| VAL | O | A | 60 | 5.4 | −23.7 | 11.8 | 36 |
| GLY | N | A | 61 | 3.2 | −23.6 | 11.5 | 42 |
| GLY | CA | A | 61 | 3.3 | −23.8 | 10.0 | 44 |
| GLY | C | A | 61 | 3.4 | −25.2 | 9.5 | 48 |
| GLY | O | A | 61 | 3.4 | −26.2 | 10.2 | 47 |
| SER | N | A | 62 | 3.3 | −25.3 | 8.2 | 48 |
| SER | CA | A | 62 | 3.4 | −26.6 | 7.4 | 50 |
| SER | CB | A | 62 | 2.0 | −26.9 | 6.9 | 50 |
| SER | OG | A | 62 | 1.0 | −27.0 | 7.9 | 52 |
| SER | C | A | 62 | 4.4 | −26.4 | 6.3 | 48 |
| SER | O | A | 62 | 4.2 | −25.7 | 5.4 | 46 |
| PRO | N | A | 63 | 5.5 | −27.2 | 6.4 | 48 |
| PRO | CD | A | 63 | 6.7 | −27.0 | 5.6 | 45 |
| PRO | CA | A | 63 | 5.8 | −28.1 | 7.5 | 47 |
| PRO | CB | A | 63 | 7.0 | −28.9 | 6.9 | 43 |
| PRO | CG | A | 63 | 7.8 | −27.7 | 6.4 | 46 |
| PRO | C | A | 63 | 6.1 | −27.3 | 8.8 | 45 |
| PRO | O | A | 63 | 6.6 | −26.2 | 8.8 | 44 |
| PRO | N | A | 64 | 5.8 | −27.9 | 10.0 | 43 |
| PRO | CD | A | 64 | 5.7 | −29.4 | 10.1 | 41 |
| PRO | CA | A | 64 | 6.0 | −27.4 | 11.3 | 42 |
| PRO | CB | A | 64 | 5.5 | −28.5 | 12.2 | 39 |
| PRO | CG | A | 64 | 6.2 | −29.6 | 11.6 | 38 |
| PRO | C | A | 64 | 7.4 | −26.8 | 11.7 | 41 |
| PRO | O | A | 64 | 8.4 | −27.5 | 11.5 | 41 |
| GLN | N | A | 65 | 7.4 | −25.5 | 12.1 | 39 |
| GLN | CA | A | 65 | 8.6 | −24.9 | 12.5 | 37 |
| GLN | CB | A | 65 | 8.7 | −23.5 | 11.9 | 36 |
| GLN | CG | A | 65 | 8.6 | −23.5 | 10.4 | 37 |
| GLN | CD | A | 65 | 8.7 | −22.0 | 9.9 | 38 |
| GLN | OE1 | A | 65 | 9.1 | −21.1 | 10.6 | 40 |
| GLN | NE2 | A | 65 | 8.4 | −21.9 | 8.6 | 40 |
| GLN | C | A | 65 | 8.7 | −24.8 | 14.0 | 37 |
| GLN | O | A | 65 | 7.8 | −24.4 | 14.7 | 39 |
| THR | N | A | 66 | 9.8 | −25.4 | 14.5 | 36 |
| THR | CA | A | 66 | 10.0 | −25.4 | 16.0 | 35 |
| THR | CB | A | 66 | 11.0 | −26.6 | 16.4 | 37 |
| THR | OG1 | A | 66 | 10.4 | −27.8 | 15.9 | 38 |
| THR | CG2 | A | 66 | 11.2 | −26.6 | 17.9 | 38 |
| THR | C | A | 66 | 10.5 | −24.1 | 16.6 | 35 |
| THR | O | A | 66 | 11.4 | −23.5 | 16.1 | 35 |
| LEU | N | A | 67 | 9.8 | −23.7 | 17.6 | 33 |
| LEU | CA | A | 67 | 10.0 | −22.4 | 18.3 | 32 |
| LEU | CB | A | 67 | 9.3 | −21.2 | 17.7 | 33 |
| LEU | CG | A | 67 | 9.6 | −21.0 | 16.3 | 38 |
| LEU | CD1 | A | 67 | 8.7 | −19.9 | 15.6 | 39 |
| LEU | CD2 | A | 67 | 11.1 | −20.6 | 16.1 | 37 |
| LEU | C | A | 67 | 9.7 | −22.5 | 19.8 | 28 |
| LEU | O | A | 67 | 8.7 | −23.1 | 20.2 | 29 |
| ASN | N | A | 68 | 10.6 | −21.9 | 20.7 | 30 |
| ASN | CA | A | 68 | 10.3 | −21.9 | 22.1 | 30 |
| ASN | CE | A | 68 | 11.6 | −21.9 | 23.0 | 30 |
| ASH | CG | A | 68 | 12.4 | −23.1 | 22.8 | 32 |
| ASN | OD1 | A | 68 | 13.6 | −23.1 | 22.6 | 37 |
| ASN | ND2 | A | 68 | 11.8 | −24.3 | 23.0 | 25 |
| ASN | C | A | 68 | 9.4 | −20.7 | 22.5 | 29 |
| ASN | O | A | 68 | 9.6 | −19.6 | 22.1 | 28 |
| ILE | N | A | 69 | 8.3 | −21.0 | 23.2 | 26 |
| ILE | CA | A | 69 | 7.3 | −20.0 | 23.5 | 26 |
| ILE | CB | A | 69 | 6.0 | −20.3 | 22.8 | 21 |
| ILE | CG2 | A | 69 | 4.9 | −19.2 | 23.1 | 19 |
| ILE | CG1 | A | 69 | 6.2 | −20.5 | 21.3 | 20 |
| ILE | CD1 | A | 69 | 6.7 | −19.2 | 20.6 | 26 |
| ILE | C | A | 69 | 7.1 | −19.8 | 25.0 | 27 |
| ILE | O | A | 69 | 6.7 | −20.7 | 25.7 | 27 |
| LEU | N | A | 70 | 7.4 | −18.5 | 25.4 | 27 |
| LEU | CA | A | 70 | 7.2 | −18.1 | 26.8 | 27 |
| LEU | CE | A | 70 | 7.9 | −16.8 | 27.1 | 28 |
| LEU | CG | A | 70 | 7.8 | −16.2 | 28.5 | 32 |
| LEU | CD1 | A | 70 | 8.7 | −15.0 | 28.7 | 33 |
| LEU | CD2 | A | 70 | 6.4 | −15.9 | 28.9 | 33 |
| LEU | C | A | 70 | 5.8 | −18.2 | 27.2 | 28 |
| LEU | O | A | 70 | 4.9 | −17.6 | 26.6 | 29 |
| VAL | N | A | 71 | 5.5 | −18.9 | 28.3 | 29 |
| VAL | CA | A | 71 | 4.1 | −19.0 | 28.8 | 31 |
| VAL | CB | A | 71 | 3.9 | −20.3 | 29.6 | 31 |
| VAL | CG1 | A | 71 | 2.5 | −20.5 | 30.1 | 33 |
| VAL | CG2 | A | 71 | 4.3 | −21.5 | 28.8 | 33 |
| VAL | C | A | 71 | 3.8 | −17.8 | 29.7 | 31 |
| VAL | O | A | 71 | 4.3 | −17.6 | 30.7 | 32 |
| ASP | N | A | 72 | 2.9 | −17.0 | 29.2 | 35 |
| ASP | CA | A | 72 | 2.5 | −15.7 | 29.8 | 29 |
| ASP | CB | A | 72 | 2.9 | −14.5 | 29.0 | 31 |
| ASP | CG | A | 72 | 2.6 | −13.2 | 29.6 | 34 |
| ASP | OD1 | A | 72 | 2.6 | −13.2 | 30.9 | 33 |
| ASP | OD2 | A | 72 | 2.4 | −12.2 | 28.9 | 32 |
| ASP | C | A | 72 | 1.0 | −15.6 | 30.2 | 28 |
| ASP | O | A | 72 | 0.2 | −15.4 | 29.3 | 28 |
| THR | N | A | 73 | 0.7 | −15.8 | 31.5 | 27 |
| THR | CA | A | 73 | −0.7 | −15.7 | 31.9 | 25 |
| THR | CB | A | 73 | −1.0 | −16.6 | 33.1 | 27 |
| THR | OG1 | A | 73 | −0.2 | −16.1 | 34.2 | 28 |
| THR | CG2 | A | 73 | −0.8 | −18.1 | 32.9 | 22 |
| THR | C | A | 73 | −1.1 | −14.3 | 32.1 | 27 |
| THR | O | A | 73 | −2.3 | −14.0 | 32.4 | 26 |
| GLY | N | A | 74 | −0.2 | −13.4 | 31.8 | 29 |
| GLY | CA | A | 74 | −0.4 | −12.0 | 32.0 | 27 |
| GLY | C | A | 74 | −0.9 | −11.2 | 30.8 | 27 |
| GLY | O | A | 74 | −1.2 | −10.0 | 30.8 | 29 |
| SER | N | A | 75 | −1.0 | −11.9 | 29.6 | 27 |
| SER | CA | A | 75 | −1.4 | −11.3 | 28.4 | 28 |
| SER | CB | A | 75 | −0.3 | −10.8 | 27.5 | 24 |
| SER | OG | A | 75 | 0.5 | −11.9 | 27.1 | 26 |
| SER | C | A | 75 | −2.3 | −12.3 | 27.6 | 30 |
| SER | O | A | 75 | −2.4 | −13.5 | 27.9 | 32 |
| SER | N | A | 76 | −3.0 | −11.8 | 26.5 | 34 |
| SER | CA | A | 76 | −3.9 | −12.6 | 25.7 | 34 |
| SER | CB | A | 76 | −5.3 | −12.0 | 25.8 | 34 |
| SER | OG | A | 76 | −5.7 | −12.1 | 27.2 | 36 |
| SER | C | A | 76 | −3.5 | −12.8 | 24.2 | 34 |
| SER | O | A | 76 | −4.2 | −13.3 | 23.4 | 31 |
| ASN | N | A | 77 | −2.3 | −12.4 | 23.9 | 31 |
| ASN | CA | A | 77 | −1.7 | −12.5 | 22.5 | 28 |
| ASN | CB | A | 77 | −1.3 | −11.2 | 21.9 | 29 |
| ASN | CG | A | 77 | −2.4 | −10.2 | 21.7 | 30 |
| ASN | OD1 | A | 77 | −2.7 | −9.4 | 22.5 | 32 |
| ASN | ND2 | A | 77 | −3.0 | −10.3 | 20.5 | 32 |
| ASN | C | A | 77 | −0.7 | −13.6 | 22.3 | 30 |
| ASN | O | A | 77 | 0.2 | −13.8 | 23.1 | 25 |
| PHE | N | A | 78 | −0.9 | −14.4 | 21.2 | 32 |
| PHE | CA | A | 78 | 0.0 | −15.4 | 20.8 | 30 |
| PHE | CB | A | 78 | −0.7 | −16.6 | 20.1 | 29 |
| PHE | CG | A | 78 | 0.3 | −17.7 | 19.7 | 34 |
| PHE | CD1 | A | 78 | 0.2 | −18.2 | 18.4 | 34 |
| PHE | CD2 | A | 78 | 1.3 | −18.1 | 20.6 | 33 |
| PHE | CE1 | A | 78 | 1.1 | −19.2 | 18.0 | 35 |
| PHE | CE2 | A | 78 | 2.2 | −19.1 | 20.1 | 32 |
| PHE | CZ | A | 78 | 2.1 | −19.6 | 18.9 | 30 |
| PHE | C | A | 78 | 0.9 | −14.7 | 19.7 | 33 |
| PHE | O | A | 78 | 0.4 | −14.2 | 18.7 | 34 |
| ALA | N | A | 79 | 2.2 | −14.6 | 20.0 | 31 |
| ALA | CA | A | 79 | 3.1 | −13.9 | 19.1 | 29 |
| ALA | CB | A | 79 | 3.2 | −12.4 | 19.5 | 27 |
| ALA | C | A | 79 | 4.5 | −14.5 | 19.0 | 30 |
| ALA | O | A | 79 | 5.0 | −15.0 | 19.9 | 33 |
| VAL | N | A | 80 | 5.0 | −14.5 | 17.8 | 28 |
| VAL | CA | A | 80 | 6.4 | −15.1 | 17.5 | 28 |
| VAL | CB | A | 80 | 6.3 | −16.5 | 16.9 | 26 |
| VAL | CG1 | A | 80 | 5.5 | −17.4 | 17.7 | 22 |
| VAL | CG2 | A | 80 | 5.7 | −16.3 | 15.4 | 24 |
| VAL | C | A | 80 | 7.2 | −14.2 | 16.7 | 27 |
| VAL | O | A | 80 | 6.7 | −13.5 | 15.8 | 27 |
| GLY | N | A | 81 | 8.5 | −14.1 | 17.0 | 25 |
| GLY | CA | A | 81 | 9.4 | −13.3 | 16.2 | 26 |
| GLY | C | A | 81 | 9.3 | −13.8 | 14.8 | 32 |
| GLY | O | A | 81 | 9.4 | −15.0 | 14.6 | 30 |
| ALA | N | A | 82 | 9.2 | −12.9 | 13.9 | 34 |
| ALA | CA | A | 82 | 9.0 | −13.2 | 12.5 | 35 |
| ALA | CB | A | 82 | 7.6 | −12.9 | 12.0 | 35 |
| ALA | C | A | 82 | 10.1 | −12.5 | 11.6 | 36 |
| ALA | O | A | 82 | 10.0 | −12.6 | 10.4 | 40 |
| ALA | N | A | 83 | 11.1 | −11.9 | 12.2 | 35 |
| ALA | CA | A | 83 | 12.1 | −11.2 | 11.5 | 37 |
| ALA | CB | A | 83 | 11.7 | −9.8 | 11.3 | 34 |
| ALA | C | A | 83 | 13.4 | −11.3 | 12.3 | 39 |
| ALA | O | A | 83 | 13.4 | −11.4 | 13.5 | 42 |
| PRO | N | A | 84 | 14.6 | −11.3 | 11.6 | 38 |
| PRO | CD | A | 84 | 14.8 | −10.7 | 10.3 | 37 |
| PRO | CA | A | 84 | 15.8 | −11.4 | 12.3 | 35 |
| PRO | CB | A | 84 | 16.8 | −11.3 | 11.2 | 37 |
| PRO | CG | A | 84 | 16.3 | −10.2 | 10.4 | 40 |
| PRO | C | A | 84 | 16.0 | −10.4 | 13.4 | 36 |
| PRO | O | A | 84 | 15.6 | −9.2 | 13.2 | 35 |
| HIS | N | A | 85 | 16.6 | −10.8 | 14.5 | 36 |
| HIS | CA | A | 85 | 16.8 | −10.0 | 15.7 | 35 |
| HIS | CB | A | 85 | 15.5 | −10.0 | 16.6 | 34 |
| HIS | CG | A | 85 | 15.7 | −9.1 | 17.8 | 35 |
| HIS | CD2 | A | 85 | 15.1 | −7.9 | 18.1 | 35 |
| HIS | ND1 | A | 85 | 16.5 | −9.5 | 18.8 | 35 |
| HIS | CE1 | A | 85 | 16.4 | −8.5 | 19.8 | 34 |
| HIS | NE2 | A | 85 | 15.6 | −7.6 | 19.4 | 35 |
| HIS | C | A | 85 | 18.0 | −10.5 | 16.4 | 37 |
| HIS | O | A | 85 | 18.2 | −11.7 | 16.5 | 37 |
| PRO | N | A | 86 | 18.8 | −9.6 | 16.9 | 36 |
| PRO | CD | A | 86 | 18.8 | −8.1 | 16.7 | 36 |
| PRO | CA | A | 86 | 20.1 | −9.9 | 17.7 | 32 |
| PRO | CB | A | 86 | 20.4 | −8.5 | 18.3 | 32 |
| PRO | CG | A | 86 | 20.2 | −7.7 | 17.1 | 37 |
| PRO | C | A | 86 | 20.0 | −11.0 | 18.7 | 33 |
| PRO | O | A | 86 | 20.9 | −11.7 | 19.0 | 33 |
| PHE | N | A | 87 | 18.8 | −11.2 | 19.2 | 30 |
| PHE | CA | A | 87 | 18.6 | −12.2 | 20.3 | 30 |
| PHE | CB | A | 87 | 17.8 | −11.6 | 21.5 | 29 |
| PHE | CG | A | 87 | 18.5 | −10.4 | 22.1 | 35 |
| PHE | CD1 | A | 87 | 17.9 | −9.6 | 23.0 | 34 |
| PHE | CD2 | A | 87 | 19.9 | −10.1 | 21.8 | 37 |
| PHE | CE1 | A | 87 | 18.5 | −8.5 | 23.6 | 34 |
| PHE | CE2 | A | 87 | 20.5 | −9.0 | 22.3 | 38 |
| PHE | CZ | A | 87 | 19.9 | −8.2 | 23.2 | 37 |
| PHE | C | A | 87 | 17.9 | −13.4 | 19.8 | 30 |
| PHE | O | A | 87 | 17.6 | −14.4 | 20.6 | 32 |
| LEU | N | A | 88 | 17.5 | −13.5 | 18.5 | 25 |
| LEU | CA | A | 88 | 16.8 | −14.7 | 18.0 | 25 |
| LEU | CB | A | 88 | 15.6 | −14.3 | 17.2 | 23 |
| LEU | CG | A | 88 | 14.5 | −13.5 | 17.9 | 27 |
| LEU | CD1 | A | 88 | 13.4 | −13.1 | 17.0 | 24 |
| LEU | CD2 | A | 88 | 13.9 | −14.3 | 19.1 | 23 |
| LEU | C | A | 88 | 17.7 | −15.6 | 17.2 | 27 |
| LEU | O | A | 88 | 18.3 | −15.2 | 16.1 | 29 |
| HIS | N | A | 89 | 17.9 | −16.8 | 17.6 | 32 |
| HIS | CA | A | 89 | 18.7 | −17.8 | 16.9 | 32 |
| HIS | CB | A | 89 | 19.3 | −18.9 | 17.7 | 36 |
| HIS | CG | A | 89 | 20.2 | −18.4 | 18.8 | 41 |
| HIS | CD2 | A | 89 | 20.4 | −17.2 | 19.3 | 46 |
| HIS | ND1 | A | 89 | 21.0 | −19.2 | 19.5 | 46 |
| HIS | CE1 | A | 89 | 21.7 | −18.5 | 20.4 | 45 |
| HIS | NE2 | A | 89 | 21.4 | −17.3 | 20.3 | 48 |
| HIS | C | A | 89 | 17.9 | −18.4 | 15.7 | 30 |
| HIS | O | A | 89 | 18.4 | −19.0 | 14.8 | 36 |
| ARG | N | A | 90 | 16.6 | −18.1 | 15.8 | 27 |
| ARG | CA | A | 90 | 15.6 | −18.6 | 14.8 | 26 |
| ARG | CB | A | 90 | 15.3 | −20.1 | 15.0 | 25 |
| ARG | CG | A | 90 | 14.8 | −20.5 | 16.4 | 26 |
| ARG | CD | A | 90 | 14.6 | −22.0 | 16.5 | 26 |
| ARG | NE | A | 90 | 14.1 | −22.4 | 17.8 | 26 |
| ARG | CZ | A | 90 | 13.9 | −23.7 | 18.1 | 26 |
| ARG | NH1 | A | 90 | 14.3 | −24.6 | 17.3 | 22 |
| ARG | NH2 | A | 90 | 13.5 | −24.0 | 19.3 | 26 |
| ARG | C | A | 90 | 14.3 | −17.8 | 15.0 | 28 |
| ARG | O | A | 90 | 14.0 | −17.3 | 16.0 | 31 |
| TYR | N | A | 91 | 13.6 | −17.8 | 13.9 | 29 |
| TYR | CA | A | 91 | 12.3 | −17.0 | 13.9 | 30 |
| TYR | CB | A | 91 | 12.6 | −15.5 | 13.6 | 32 |
| TYR | CG | A | 91 | 13.2 | −15.2 | 12.3 | 35 |
| TYR | CD1 | A | 91 | 12.5 | −15.1 | 11.1 | 35 |
| TYR | CE1 | A | 91 | 13.2 | −14.9 | 9.9 | 40 |
| TYR | CD2 | A | 91 | 14.6 | −15.1 | 12.3 | 37 |
| TYR | CE2 | A | 91 | 15.3 | −14.8 | 11.1 | 37 |
| TYR | CZ | A | 91 | 14.6 | −14.8 | 9.9 | 38 |
| TYR | OH | A | 91 | 15.2 | −14.5 | 8.7 | 38 |
| TYR | C | A | 91 | 11.3 | −17.6 | 12.9 | 30 |
| TYR | O | A | 91 | 11.6 | −18.2 | 11.9 | 33 |
| TYR | N | A | 92 | 10.0 | −17.3 | 13.2 | 31 |
| TYR | CA | A | 92 | 8.9 | −17.7 | 12.3 | 29 |
| TYR | CB | A | 92 | 7.6 | −17.3 | 13.0 | 31 |
| TYR | CG | A | 92 | 6.3 | −17.6 | 12.2 | 33 |
| TYR | CD1 | A | 92 | 6.2 | −18.9 | 11.5 | 30 |
| TYR | CE1 | A | 92 | 5.0 | −19.2 | 10.9 | 29 |
| TYR | CD2 | A | 92 | 5.2 | −16.8 | 12.2 | 31 |
| TYR | CE2 | A | 92 | 4.0 | −17.1 | 11.5 | 29 |
| TYR | CZ | A | 92 | 3.9 | −18.3 | 10.9 | 29 |
| TYR | OH | A | 92 | 2.7 | −18.7 | 10.3 | 34 |
| TYR | C | A | 92 | 9.0 | −17.2 | 10.9 | 32 |
| TYR | O | A | 92 | 9.0 | −16.0 | 10.6 | 33 |
| GLN | N | A | 93 | 9.1 | −18.1 | 9.9 | 35 |
| GLN | CA | A | 93 | 9.2 | −17.8 | 8.5 | 39 |
| GLN | CB | A | 93 | 10.5 | −18.2 | 7.9 | 45 |
| GLN | CG | A | 93 | 11.7 | −17.7 | 8.6 | 53 |
| GLN | CD | A | 93 | 13.0 | −18.1 | 7.9 | 52 |
| GLN | OE1 | A | 93 | 13.8 | −18.8 | 8.5 | 56 |
| GLN | NE2 | A | 93 | 13.1 | −17.8 | 6.6 | 52 |
| GLN | C | A | 93 | 8.0 | −18.1 | 7.8 | 38 |
| GLN | O | A | 93 | 7.8 | −19.3 | 7.4 | 36 |
| ARG | N | A | 94 | 7.1 | −17.1 | 7.6 | 41 |
| ARG | CA | A | 94 | 5.8 | −17.2 | 6.9 | 46 |
| ARG | CB | A | 94 | 5.1 | −15.9 | 7.1 | 44 |
| ARG | CG | A | 94 | 4.8 | −15.6 | 8.6 | 43 |
| ARG | CD | A | 94 | 4.2 | −14.2 | 8.8 | 40 |
| ARG | NE | A | 94 | 5.1 | −13.2 | 8.3 | 40 |
| ARG | CZ | A | 94 | 4.9 | −11.9 | 8.3 | 41 |
| ARG | NH1 | A | 94 | 3.7 | −11.4 | 8.7 | 42 |
| ARG | NH2 | A | 94 | 5.9 | −11.0 | 8.0 | 39 |
| ARG | C | A | 94 | 5.9 | −17.6 | 5.5 | 50 |
| ARG | O | A | 94 | 4.9 | −18.1 | 4.9 | 51 |
| GLN | N | A | 95 | 7.0 | −17.5 | 4.8 | 53 |
| GLN | CA | A | 95 | 7.2 | −17.8 | 3.4 | 58 |
| GLN | CB | A | 95 | 8.2 | −16.9 | 2.7 | 64 |
| CLN | CG | A | 95 | 7.9 | −15.4 | 2.7 | 75 |
| GLN | CD | A | 95 | 9.0 | −14.7 | 2.0 | 80 |
| GLN | OE1 | A | 95 | 10.0 | −15.3 | 1.5 | 83 |
| GLN | NE2 | A | 95 | 8.9 | −13.4 | 2.0 | 83 |
| GLN | C | A | 95 | 7.5 | −19.3 | 3.2 | 55 |
| GLN | O | A | 95 | 7.5 | −19.8 | 2.1 | 55 |
| LEU | N | A | 96 | 7.7 | −20.0 | 4.3 | 50 |
| LEU | CA | A | 96 | 8.0 | −21.4 | 4.3 | 46 |
| LEU | CB | A | 96 | 9.3 | −21.7 | 5.1 | 44 |
| LEU | CG | A | 96 | 10.5 | −21.0 | 4.6 | 46 |
| LEU | CD1 | A | 96 | 11.7 | −21.3 | 5.5 | 45 |
| LEU | CD2 | A | 96 | 10.8 | −21.3 | 3.1 | 46 |
| LEU | C | A | 96 | 6.9 | −22.3 | 4.7 | 47 |
| LEU | O | A | 96 | 7.0 | −23.5 | 4.8 | 47 |
| SER | N | A | 97 | 5.8 | −21.6 | 5.1 | 47 |
| SER | CA | A | 97 | 4.6 | −22.3 | 5.6 | 46 |
| SER | CB | A | 97 | 4.1 | −21.8 | 6.9 | 46 |
| SER | OG | A | 97 | 3.0 | −22.5 | 7.4 | 49 |
| SER | C | A | 97 | 3.5 | −22.3 | 4.5 | 45 |
| SER | O | A | 97 | 2.9 | −21.3 | 4.2 | 41 |
| SER | N | A | 98 | 3.1 | −23.5 | 4.1 | 45 |
| SER | CA | A | 98 | 2.0 | −23.6 | 3.1 | 49 |
| SER | CB | A | 98 | 2.1 | −24.9 | 2.4 | 51 |
| SER | OG | A | 98 | 2.0 | −26.0 | 3.3 | 53 |
| SER | C | A | 98 | 0.6 | −23.4 | 3.7 | 52 |
| SER | O | A | 98 | −0.4 | −23.2 | 3.0 | 53 |
| THR | N | A | 99 | 0.6 | −23.3 | 5.1 | 50 |
| THR | CA | A | 99 | −0.7 | −23.1 | 5.7 | 46 |
| THR | CB | A | 99 | −1.0 | −24.2 | 6.8 | 48 |
| THR | OG1 | A | 99 | 0.1 | −24.3 | 7.7 | 49 |
| THR | CG2 | A | 99 | −1.3 | −25.5 | 6.1 | 48 |
| THR | C | A | 99 | −0.9 | −21.7 | 6.3 | 42 |
| THR | O | A | 99 | −1.9 | −21.4 | 6.9 | 43 |
| TYR | N | A | 100 | 0.1 | −20.8 | 6.0 | 44 |
| TYR | CA | A | 100 | 0.0 | −19.4 | 6.5 | 43 |
| TYR | CB | A | 100 | 1.4 | −18.7 | 6.2 | 41 |
| TYR | CG | A | 100 | 1.3 | −17.2 | 6.5 | 42 |
| TYR | CD1 | A | 100 | 1.1 | −16.7 | 7.8 | 40 |
| TYR | CE1 | A | 100 | 1.0 | −15.3 | 8.0 | 40 |
| TYR | CD2 | A | 100 | 1.5 | −16.3 | 5.4 | 41 |
| TYR | CE2 | A | 100 | 1.4 | −15.0 | 5.6 | 44 |
| TYR | CZ | A | 100 | 1.1 | −14.5 | 6.9 | 42 |
| TYR | OH | A | 100 | 1.0 | −13.1 | 7.1 | 42 |
| TYR | C | A | 100 | −1.1 | −18.6 | 5.8 | 47 |
| TYR | O | A | 100 | −1.3 | −18.7 | 4.6 | 48 |
| ARG | N | A | 101 | −1.8 | −17.8 | 6.6 | 46 |
| ARG | CA | A | 101 | −2.9 | −17.0 | 6.1 | 49 |
| ARG | CB | A | 101 | −4.3 | −17.5 | 6.4 | 50 |
| ARG | CG | A | 101 | −4.5 | −18.9 | 5.9 | 50 |
| ARG | CD | A | 101 | −5.9 | −19.4 | 6.3 | 52 |
| ARG | NE | A | 101 | −6.2 | −20.8 | 5.8 | 56 |
| ARG | CZ | A | 101 | −7.3 | −21.4 | 6.1 | 55 |
| ARG | NH1 | A | 101 | −8.2 | −20.9 | 6.8 | 54 |
| ARG | NH2 | A | 101 | −7.4 | −22.7 | 5.6 | 53 |
| ARG | C | A | 101 | −2.7 | −15.6 | 6.6 | 51 |
| ARG | O | A | 101 | −2.7 | −15.4 | 7.8 | 54 |
| ASP | N | A | 102 | −2.5 | −14.7 | 5.7 | 52 |
| ASP | CA | A | 102 | −2.3 | −13.3 | 6.0 | 52 |
| ASP | CB | A | 102 | −1.5 | −12.5 | 5.0 | 52 |
| ASP | CG | A | 102 | −1.2 | −11.1 | 5.3 | 53 |
| ASP | OD1 | A | 102 | −1.5 | −10.7 | 6.4 | 53 |
| ASP | OD2 | A | 102 | −0.7 | −10.4 | 4.4 | 55 |
| ASP | C | A | 102 | −3.6 | −12.6 | 6.3 | 52 |
| ASP | O | A | 102 | −4.5 | −12.6 | 5.5 | 52 |
| LEU | N | A | 103 | −3.7 | −12.0 | 7.5 | 52 |
| LEU | CA | A | 103 | −4.9 | −11.2 | 7.9 | 52 |
| LEU | CB | A | 103 | −5.2 | −11.3 | 9.4 | 53 |
| LEU | CG | A | 103 | −5.4 | −12.8 | 9.9 | 54 |
| LEU | CD1 | A | 103 | −5.5 | −12.8 | 11.4 | 52 |
| LEU | CD2 | A | 103 | −6.6 | −13.4 | 9.2 | 54 |
| LEU | C | A | 103 | −4.9 | −9.8 | 7.4 | 56 |
| LEU | O | A | 103 | −5.9 | −9.0 | 7.6 | 57 |
| ARG | N | A | 104 | −3.8 | −9.4 | 6.8 | 59 |
| ARG | CA | A | 104 | −3.7 | −8.0 | 6.2 | 65 |
| ARG | CB | A | 104 | −4.7 | −7.8 | 5.1 | 68 |
| ARG | CG | A | 104 | −4.5 | −8.8 | 3.9 | 72 |
| ARG | CD | A | 104 | −5.5 | −8.6 | 2.8 | 76 |
| ARG | NE | A | 104 | −5.5 | −7.3 | 2.2 | 80 |
| ARG | CZ | A | 104 | −6.3 | −6.3 | 2.5 | 82 |
| ARG | NH1 | A | 104 | −7.1 | −6.4 | 3.5 | 83 |
| ARG | NH2 | A | 104 | −6.2 | −5.1 | 1.9 | 81 |
| ARG | C | A | 104 | −3.9 | −6.9 | 7.3 | 65 |
| ARG | O | A | 104 | −4.0 | −5.7 | 6.9 | 67 |
| LYS | N | A | 105 | −3.8 | −7.2 | 8.6 | 63 |
| LYS | CA | A | 105 | −3.9 | −6.2 | 9.6 | 60 |
| LYS | CB | A | 105 | −5.3 | −6.3 | 10.3 | 59 |
| LYS | CG | A | 105 | −5.4 | −5.3 | 11.4 | 62 |
| LYS | CD | A | 105 | −6.8 | −5.3 | 12.1 | 64 |
| LYS | CE | A | 105 | −7.2 | −6.7 | 12.7 | 66 |
| LYS | NZ | A | 105 | −8.5 | −6.7 | 13.3 | 64 |
| LYS | C | A | 105 | −2.8 | −6.2 | 10.7 | 57 |
| LYS | O | A | 105 | −2.4 | −7.3 | 11.2 | 57 |
| GLY | N | A | 106 | −2.2 | −5.1 | 10.9 | 53 |
| GLY | CA | A | 106 | −1.2 | −4.9 | 11.9 | 51 |
| GLY | C | A | 106 | −1.6 | −5.1 | 13.4 | 49 |
| GLY | O | A | 106 | −2.8 | −5.2 | 13.7 | 50 |
| VAL | N | A | 107 | −0.6 | −5.2 | 14.2 | 43 |
| VAL | CA | A | 107 | −0.8 | −5.4 | 15.7 | 39 |
| VAL | CB | A | 107 | −1.1 | −6.9 | 16.0 | 39 |
| VAL | CG1 | A | 107 | 0.1 | −7.7 | 15.6 | 34 |
| VAL | CG2 | A | 107 | −1.5 | −7.0 | 17.5 | 34 |
| VAL | C | A | 107 | 0.3 | −4.8 | 16.5 | 39 |
| VAL | O | A | 107 | 1.5 | −5.0 | 16.2 | 39 |
| TYR | N | A | 108 | −0.1 | −4.2 | 17.6 | 39 |
| TYR | CA | A | 108 | 0.8 | −3.6 | 18.6 | 42 |
| TYR | CB | A | 108 | 0.7 | −2.1 | 18.5 | 46 |
| TYR | CG | A | 108 | 1.5 | −1.4 | 19.6 | 50 |
| TYR | CD1 | A | 108 | 2.9 | −1.5 | 19.7 | 53 |
| TYR | CE1 | A | 108 | 3.6 | −0.9 | 20.7 | 52 |
| TYR | CD2 | A | 108 | 0.9 | −0.5 | 20.5 | 50 |
| TYR | CE2 | A | 108 | 1.6 | 0.1 | 21.5 | 51 |
| TYR | CZ | A | 108 | 3.0 | −0.1 | 21.6 | 52 |
| TYR | OH | A | 108 | 3.7 | 0.6 | 22.6 | 52 |
| TYR | C | A | 108 | 0.5 | −4.1 | 20.0 | 39 |
| TYR | O | A | 108 | −0.7 | −3.9 | 20.5 | 40 |
| VAL | N | A | 109 | 1.4 | −4.7 | 20.7 | 36 |
| VAL | CA | A | 109 | 1.2 | −5.1 | 22.0 | 35 |
| VAL | CB | A | 109 | 1.1 | −6.7 | 22.2 | 32 |
| VAL | CG1 | A | 109 | 1.0 | −7.1 | 23.7 | 31 |
| VAL | CG2 | A | 109 | −0.1 | −7.2 | 21.4 | 32 |
| VAL | C | A | 109 | 2.3 | −4.6 | 23.0 | 36 |
| VAL | O | A | 109 | 3.5 | −5.0 | 22.9 | 35 |
| PRO | N | A | 110 | 1.9 | −3.7 | 23.9 | 35 |
| PRO | CD | A | 110 | 0.6 | −3.0 | 23.9 | 30 |
| PRO | CA | A | 110 | 2.8 | −3.1 | 24.9 | 33 |
| PRO | CB | A | 110 | 2.3 | −1.7 | 24.9 | 35 |
| PRO | CG | A | 110 | 0.8 | −1.9 | 25.0 | 32 |
| PRO | C | A | 110 | 2.6 | −3.8 | 26.3 | 32 |
| PRO | O | A | 110 | 1.5 | −4.1 | 26.7 | 31 |
| TYR | N | A | 111 | 3.8 | −4.1 | 26.9 | 32 |
| TYR | CA | A | 111 | 3.8 | −4.8 | 28.2 | 33 |
| TYR | CB | A | 111 | 4.7 | −6.0 | 28.2 | 30 |
| TYR | CG | A | 111 | 4.2 | −7.0 | 27.1 | 25 |
| TYR | CD1 | A | 111 | 3.3 | −8.0 | 27.5 | 25 |
| TYR | CE1 | A | 111 | 2.9 | −9.0 | 26.5 | 24 |
| TYR | CD2 | A | 111 | 4.7 | −7.0 | 25.9 | 26 |
| TYR | CE2 | A | 111 | 4.3 | −8.0 | 24.9 | 26 |
| TYR | CZ | A | 111 | 3.4 | −8.9 | 25.3 | 24 |
| TYR | OH | A | 111 | 3.0 | −9.9 | 24.3 | 27 |
| TYR | C | A | 111 | 4.3 | −3.8 | 29.3 | 33 |
| TYR | O | A | 111 | 4.8 | −2.7 | 29.0 | 35 |
| THR | N | A | 112 | 4.3 | −4.3 | 30.6 | 29 |
| THR | CA | A | 112 | 4.8 | −3.4 | 31.7 | 25 |
| THR | CB | A | 112 | 4.7 | −4.2 | 33.0 | 22 |
| THR | OG1 | A | 112 | 3.3 | −4.4 | 33.3 | 24 |
| THR | CG2 | A | 112 | 5.3 | −3.4 | 34.1 | 23 |
| THR | C | A | 112 | 6.3 | −3.1 | 31.4 | 28 |
| THR | O | A | 112 | 6.8 | −2.1 | 31.8 | 31 |
| GLN | N | A | 113 | 6.9 | −4.0 | 30.6 | 31 |
| GLN | CA | A | 113 | 8.3 | −3.8 | 30.2 | 32 |
| GLN | CB | A | 113 | 9.4 | −4.4 | 31.2 | 36 |
| GLN | CG | A | 113 | 9.4 | −3.6 | 32.6 | 51 |
| GLN | CD | A | 113 | 9.7 | −2.1 | 32.3 | 57 |
| GLN | OE1 | A | 113 | 10.1 | −1.8 | 31.2 | 61 |
| GLN | NE2 | A | 113 | 9.7 | −1.3 | 33.3 | 59 |
| GLN | C | A | 113 | 8.4 | −4.6 | 28.9 | 31 |
| GLN | O | A | 113 | 8.2 | −5.9 | 28.9 | 29 |
| GLY | N | A | 114 | 8.7 | −3.9 | 27.8 | 23 |
| GLY | CA | A | 114 | 8.8 | −4.6 | 26.6 | 23 |
| GLY | C | A | 114 | 7.6 | −4.3 | 25.7 | 30 |
| GLY | O | A | 114 | 6.5 | −4.2 | 26.3 | 26 |
| LYS | N | A | 115 | 7.8 | −4.3 | 24.4 | 31 |
| LYS | CA | A | 115 | 6.7 | −4.0 | 23.5 | 33 |
| LYS | CB | A | 115 | 6.4 | −2.5 | 23.4 | 35 |
| LYS | CG | A | 115 | 7.6 | −1.6 | 23.0 | 42 |
| LYS | CD | A | 115 | 7.2 | −0.2 | 22.9 | 44 |
| LYS | CE | A | 115 | 8.4 | 0.7 | 22.5 | 50 |
| LYS | NZ | A | 115 | 8.1 | 2.1 | 22.4 | 54 |
| LYS | C | A | 115 | 7.1 | −4.5 | 22.1 | 30 |
| LYS | O | A | 115 | 8.3 | −4.6 | 21.8 | 32 |
| TRP | N | A | 116 | 6.2 | −5.0 | 21.4 | 29 |
| TRP | CA | A | 116 | 6.4 | −5.4 | 20.0 | 33 |
| TRP | CB | A | 116 | 6.7 | −7.0 | 20.0 | 30 |
| TRP | CG | A | 116 | 5.6 | −7.8 | 20.5 | 29 |
| TRP | CD2 | A | 116 | 4.4 | −8.1 | 19.9 | 30 |
| TRP | CE2 | A | 116 | 3.7 | −9.0 | 20.8 | 28 |
| TRP | CE3 | A | 116 | 3.7 | −7.7 | 18.7 | 27 |
| TRP | CD1 | A | 116 | 5.6 | −8.5 | 21.7 | 29 |
| TRP | NE1 | A | 116 | 4.5 | −9.2 | 21.8 | 32 |
| TRP | CZ2 | A | 116 | 2.4 | −9.4 | 20.5 | 28 |
| TRP | CZ3 | A | 116 | 2.5 | −8.2 | 18.4 | 25 |
| TRP | CH2 | A | 116 | 1.8 | −9.0 | 19.3 | 25 |
| TRP | C | A | 116 | 5.3 | −5.1 | 19.1 | 35 |
| TRP | O | A | 116 | 4.2 | −4.8 | 19.5 | 36 |
| GLU | N | A | 117 | 5.7 | −5.0 | 17.8 | 40 |
| GLU | CA | A | 117 | 4.7 | −4.6 | 16.8 | 44 |
| GLU | CB | A | 117 | 4.9 | −3.2 | 16.2 | 49 |
| GLU | CG | A | 117 | 3.9 | −2.7 | 15.2 | 59 |
| GLU | CD | A | 117 | 4.3 | −1.3 | 14.8 | 66 |
| GLU | OE1 | A | 117 | 5.3 | −0.8 | 15.3 | 66 |
| GLU | OE2 | A | 117 | 3.6 | −0.8 | 13.9 | 71 |
| GLU | C | A | 117 | 4.8 | −5.7 | 15.7 | 39 |
| GLU | O | A | 117 | 5.9 | −6.1 | 15.3 | 38 |
| GLY | N | A | 118 | 3.6 | −6.2 | 15.2 | 36 |
| GLY | CA | A | 118 | 3.7 | −7.2 | 14.2 | 36 |
| GLY | C | A | 118 | 2.6 | −7.2 | 13.2 | 35 |
| GLY | O | A | 118 | 1.8 | −6.2 | 13.0 | 35 |
| GLU | N | A | 119 | 2.4 | −8.4 | 12.5 | 33 |
| GLU | CA | A | 119 | 1.4 | −8.5 | 11.5 | 32 |
| GLU | CB | A | 119 | 2.1 | −8.6 | 10.1 | 36 |
| GLU | CG | A | 119 | 3.0 | −7.4 | 9.9 | 42 |
| GLU | CD | A | 119 | 3.7 | −7.5 | 8.5 | 45 |
| GLU | OE1 | A | 119 | 3.7 | −6.6 | 7.8 | 50 |
| GLU | OE2 | A | 119 | 4.3 | −8.6 | 8.2 | 47 |
| GLU | C | A | 119 | 0.5 | −9.7 | 11.8 | 31 |
| GLU | O | A | 119 | 1.0 | −10.8 | 12.0 | 29 |
| LEU | N | A | 120 | −0.8 | −9.5 | 11.8 | 32 |
| LEU | CA | A | 120 | −1.7 | −10.6 | 12.1 | 31 |
| LEU | CB | A | 120 | −3.1 | −10.0 | 12.5 | 34 |
| LEU | CG | A | 120 | −3.0 | −9.2 | 13.8 | 36 |
| LEU | CD1 | A | 120 | −4.3 | −8.6 | 14.2 | 37 |
| LEU | CD2 | A | 120 | −2.4 | −10.1 | 15.0 | 35 |
| LEU | C | A | 120 | −1.9 | −11.6 | 11.0 | 35 |
| LEU | O | A | 120 | −2.0 | −11.3 | 9.8 | 33 |
| GLY | N | A | 121 | −2.0 | −12.9 | 11.4 | 34 |
| GLY | CA | A | 121 | −2.2 | −14.0 | 10.5 | 31 |
| GLY | C | A | 121 | −2.7 | −15.1 | 11.3 | 35 |
| GLY | O | A | 121 | −2.9 | −15.0 | 12.5 | 36 |
| THR | N | A | 122 | −2.9 | −16.3 | 10.6 | 32 |
| THR | CA | A | 122 | −3.4 | −17.5 | 11.3 | 32 |
| THR | CB | A | 122 | −4.9 | −17.8 | 11.0 | 34 |
| THR | OG1 | A | 122 | −5.1 | −18.0 | 9.7 | 39 |
| THR | CG2 | A | 122 | −5.7 | −16.6 | 11.5 | 32 |
| THR | C | A | 122 | −2.5 | −18.6 | 10.6 | 29 |
| THR | O | A | 122 | −2.1 | −18.5 | 9.5 | 29 |
| ASP | N | A | 123 | −2.3 | −19.7 | 11.4 | 29 |
| ASP | CA | A | 123 | −1.6 | −20.8 | 10.8 | 33 |
| ASP | CB | A | 123 | −0.1 | −20.4 | 10.6 | 36 |
| ASP | CG | A | 123 | 0.7 | −21.5 | 9.8 | 40 |
| ASP | OD1 | A | 123 | 0.2 | −22.5 | 9.5 | 35 |
| ASP | OD2 | A | 123 | 1.9 | −21.2 | 9.5 | 44 |
| ASP | C | A | 123 | −1.8 | −22.0 | 11.8 | 33 |
| ASP | O | A | 123 | −2.4 | −21.8 | 12.8 | 36 |
| LEU | N | A | 124 | −1.3 | −23.2 | 11.4 | 34 |
| LEU | CA | A | 124 | −1.4 | −24.3 | 12.3 | 35 |
| LEU | CB | A | 124 | −1.4 | −25.6 | 11.5 | 36 |
| LEU | CG | A | 124 | −2.5 | −25.6 | 10.5 | 39 |
| LEU | CD1 | A | 124 | −2.5 | −26.9 | 9.6 | 40 |
| LEU | CD2 | A | 124 | −3.9 | −25.6 | 11.2 | 35 |
| LEU | C | A | 124 | −0.3 | −24.4 | 13.4 | 35 |
| LEU | O | A | 124 | 0.9 | −24.3 | 13.1 | 34 |
| VAL | N | A | 125 | −0.8 | −24.6 | 14.6 | 38 |
| VAL | CA | A | 125 | 0.1 | −24.6 | 15.8 | 35 |
| VAL | CB | A | 125 | −0.2 | −23.4 | 16.8 | 33 |
| VAL | CG1 | A | 125 | 0.8 | −23.5 | 18.0 | 31 |
| VAL | CG2 | A | 125 | −0.1 | −22.1 | 16.0 | 31 |
| VAL | C | A | 125 | −0.1 | −26.0 | 16.6 | 39 |
| VAL | O | A | 125 | −1.2 | −26.4 | 16.8 | 43 |
| SER | N | A | 126 | 1.0 | −26.6 | 17.0 | 39 |
| SER | CA | A | 126 | 1.1 | −27.8 | 17.7 | 41 |
| SER | CB | A | 126 | 1.4 | −29.0 | 16.8 | 44 |
| SER | OG | A | 126 | 0.5 | −29.2 | 15.8 | 51 |
| SER | C | A | 126 | 2.0 | −27.7 | 18.9 | 41 |
| SER | O | A | 126 | 3.0 | −27.1 | 18.9 | 43 |
| ILE | N | A | 127 | 1.6 | −28.4 | 20.0 | 39 |
| ILE | CA | A | 127 | 2.5 | −28.4 | 21.2 | 36 |
| ILE | CB | A | 127 | 1.6 | −28.0 | 22.5 | 37 |
| ILE | CG2 | A | 127 | 2.5 | −28.1 | 23.7 | 31 |
| ILE | CG1 | A | 127 | 1.0 | −26.6 | 22.3 | 38 |
| ILE | CD1 | A | 127 | 0.2 | −26.2 | 23.4 | 38 |
| ILE | C | A | 127 | 3.0 | −29.8 | 21.3 | 36 |
| ILE | O | A | 127 | 2.4 | −30.7 | 21.8 | 35 |
| PRO | N | A | 128 | 4.3 | −30.0 | 20.9 | 36 |
| PRO | CD | A | 128 | 5.1 | −29.0 | 20.1 | 36 |
| PRO | CA | A | 128 | 5.0 | −31.2 | 20.9 | 37 |
| PRO | CB | A | 128 | 6.5 | −30.8 | 20.7 | 35 |
| PRO | CG | A | 128 | 6.2 | −29.9 | 19.5 | 36 |
| PRO | C | A | 128 | 4.9 | −32.0 | 22.2 | 38 |
| PRO | O | A | 128 | 4.7 | −33.2 | 22.2 | 39 |
| HIS | N | A | 129 | 5.0 | −31.3 | 23.4 | 38 |
| HIS | CA | A | 129 | 4.9 | −32.0 | 24.7 | 39 |
| HIS | CB | A | 129 | 6.1 | −31.7 | 25.5 | 40 |
| HIS | CG | A | 129 | 7.4 | −32.2 | 24.9 | 42 |
| HIS | CD2 | A | 129 | 8.4 | −31.6 | 24.3 | 41 |
| HIS | ND1 | A | 129 | 7.7 | −33.6 | 24.8 | 41 |
| HIS | CE1 | A | 129 | 8.8 | −33.8 | 24.2 | 41 |
| HIS | NE2 | A | 129 | 9.3 | −32.6 | 23.9 | 39 |
| HIS | C | A | 129 | 3.6 | −31.6 | 25.4 | 38 |
| HIS | O | A | 129 | 3.6 | −31.5 | 26.7 | 37 |
| GLY | N | A | 130 | 2.5 | −31.4 | 24.7 | 38 |
| GLY | CA | A | 130 | 1.3 | −31.1 | 25.3 | 41 |
| GLY | C | A | 130 | 0.3 | −32.2 | 24.7 | 41 |
| GLY | O | A | 130 | 0.8 | −33.3 | 24.4 | 41 |
| PRO | N | A | 131 | −0.9 | −31.8 | 24.4 | 43 |
| PRO | CD | A | 131 | −1.6 | −30.5 | 24.6 | 42 |
| PRO | CA | A | 131 | −1.9 | −32.8 | 23.9 | 45 |
| PRO | CB | A | 131 | −3.2 | −32.2 | 24.3 | 44 |
| PRO | CG | A | 131 | −3.0 | −30.8 | 24.0 | 45 |
| PRO | C | A | 131 | −1.7 | −32.9 | 22.4 | 45 |
| PRO | O | A | 131 | −1.5 | −31.9 | 21.7 | 45 |
| ASN | N | A | 132 | −1.8 | −34.1 | 21.8 | 48 |
| ASN | CA | A | 132 | −1.7 | −34.3 | 20.4 | 53 |
| ASN | CB | A | 132 | −1.4 | −35.8 | 20.0 | 60 |
| ASN | CG | A | 132 | −0.1 | −36.3 | 20.6 | 66 |
| ASN | OD1 | A | 132 | 0.8 | −36.7 | 19.8 | 69 |
| ASN | ND2 | A | 132 | 0.0 | −36.2 | 21.9 | 70 |
| ASN | C | A | 132 | −2.9 | −33.7 | 19.6 | 51 |
| ASN | O | A | 132 | −3.8 | −34.5 | 19.4 | 50 |
| VAL | N | A | 133 | −2.7 | −32.5 | 19.2 | 49 |
| VAL | CA | A | 133 | −3.8 | −31.8 | 18.4 | 50 |
| VAL | CB | A | 133 | −4.9 | −31.2 | 19.3 | 50 |
| VAL | CG1 | A | 133 | −5.6 | −32.4 | 20.1 | 46 |
| VAL | CG2 | A | 133 | −4.4 | −30.1 | 20.3 | 48 |
| VAL | C | A | 133 | −3.2 | −30.6 | 17.7 | 50 |
| VAL | O | A | 133 | −2.1 | −30.1 | 18.1 | 52 |
| THR | N | A | 134 | −3.8 | −30.3 | 16.5 | 50 |
| THR | CA | A | 134 | −3.3 | −29.2 | 15.7 | 49 |
| THR | CB | A | 134 | −2.9 | −29.7 | 14.3 | 49 |
| THR | OG1 | A | 134 | −1.9 | −30.6 | 14.4 | 52 |
| THR | CG2 | A | 134 | −2.5 | −28.5 | 13.4 | 50 |
| THR | C | A | 134 | −4.4 | −28.2 | 15.7 | 49 |
| THR | O | A | 134 | −5.6 | −28.5 | 15.4 | 50 |
| VAL | N | A | 135 | −4.1 | −26.9 | 15.9 | 47 |
| VAL | CA | A | 135 | −5.1 | −25.9 | 16.0 | 44 |
| VAL | CB | A | 135 | −5.3 | −25.4 | 17.4 | 44 |
| VAL | CG1 | A | 135 | −6.3 | −24.3 | 17.5 | 43 |
| VAL | CG2 | A | 135 | −5.6 | −26.5 | 18.4 | 43 |
| VAL | C | A | 135 | −4.7 | −24.7 | 15.1 | 43 |
| VAL | O | A | 135 | −3.6 | −24.2 | 15.1 | 46 |
| ARG | N | A | 136 | −5.7 | −24.2 | 14.3 | 40 |
| ARG | CA | A | 136 | −5.4 | −23.0 | 13.6 | 42 |
| ARG | CB | A | 136 | −6.2 | −22.9 | 12.2 | 44 |
| ARG | CG | A | 136 | −6.0 | −21.5 | 11.6 | 46 |
| ARG | CD | A | 136 | −6.7 | −21.4 | 10.2 | 46 |
| ARG | NE | A | 136 | −6.2 | −22.4 | 9.3 | 46 |
| ARG | CZ | A | 136 | −5.0 | −22.2 | 8.6 | 46 |
| ARG | NH1 | A | 136 | −4.4 | −21.0 | 8.7 | 45 |
| ARG | NH2 | A | 136 | −4.6 | −23.1 | 7.7 | 46 |
| ARG | C | A | 136 | −5.7 | −21.8 | 14.5 | 38 |
| ARG | O | A | 136 | −6.8 | −21.6 | 14.9 | 39 |
| ALA | N | A | 137 | −4.6 | −21.1 | 14.8 | 37 |
| ALA | CA | A | 137 | −4.7 | −20.0 | 15.8 | 35 |
| ALA | CB | A | 137 | −3.9 | −20.4 | 17.0 | 34 |
| ALA | C | A | 137 | −4.2 | −18.7 | 15.2 | 35 |
| ALA | O | A | 137 | −3.4 | −18.7 | 14.3 | 34 |
| ASN | N | A | 138 | −4.7 | −17.6 | 15.8 | 34 |
| ASN | CA | A | 138 | −4.2 | −16.3 | 15.3 | 34 |
| ASN | CB | A | 138 | −5.0 | −15.2 | 15.9 | 36 |
| ASN | CG | A | 138 | −6.5 | −15.2 | 15.6 | 36 |
| ASN | OD1 | A | 138 | −6.9 | −14.6 | 14.6 | 41 |
| ASN | ND2 | A | 138 | −7.2 | −16.0 | 16.3 | 39 |
| ASN | C | A | 138 | −2.7 | −16.2 | 15.8 | 34 |
| ASN | O | A | 138 | −2.4 | −16.6 | 16.9 | 36 |
| ILE | N | A | 139 | −1.9 | −15.7 | 14.9 | 33 |
| ILE | CA | A | 139 | −0.5 | −15.6 | 15.2 | 31 |
| ILE | CB | A | 139 | 0.4 | −16.6 | 14.5 | 31 |
| ILE | CG2 | A | 139 | 1.8 | −16.4 | 14.9 | 31 |
| ILE | CG1 | A | 139 | −0.1 | −18.0 | 14.8 | 33 |
| ILE | CD1 | A | 139 | 0.7 | −19.1 | 14.1 | 33 |
| ILE | C | A | 139 | 0.0 | −14.2 | 14.9 | 34 |
| ILE | O | A | 139 | −0.1 | −13.7 | 13.8 | 39 |
| ALA | N | A | 140 | 0.6 | −13.5 | 15.9 | 31 |
| ALA | CA | A | 140 | 1.1 | −12.2 | 15.6 | 33 |
| ALA | CB | A | 140 | 1.1 | −11.3 | 16.9 | 31 |
| ALA | C | A | 140 | 2.6 | −12.4 | 15.2 | 36 |
| ALA | O | A | 140 | 3.4 | −12.9 | 16.0 | 37 |
| ALA | N | A | 141 | 2.9 | −12.0 | 14.0 | 31 |
| ALA | CA | A | 141 | 4.3 | −12.2 | 13.5 | 32 |
| ALA | CB | A | 141 | 4.3 | −12.4 | 11.9 | 31 |
| ALA | C | A | 141 | 5.0 | −10.9 | 13.9 | 30 |
| ALA | O | A | 141 | 4.8 | −9.9 | 13.3 | 31 |
| ILE | N | A | 142 | 6.0 | −11.1 | 14.8 | 27 |
| ILE | CA | A | 142 | 6.7 | −9.9 | 15.3 | 30 |
| ILE | CB | A | 142 | 7.4 | −10.2 | 16.6 | 26 |
| ILE | CG2 | A | 142 | 8.2 | −9.0 | 17.1 | 24 |
| ILE | CG1 | A | 142 | 6.3 | −10.5 | 17.7 | 27 |
| ILE | CD1 | A | 142 | 6.9 | −10.9 | 19.1 | 24 |
| ILE | C | A | 142 | 7.8 | −9.4 | 14.3 | 35 |
| ILE | O | A | 142 | 8.7 | −10.1 | 13.9 | 36 |
| THR | N | A | 143 | 7.6 | −8.1 | 13.9 | 37 |
| THR | CA | A | 143 | 8.5 | −7.5 | 12.9 | 37 |
| THR | CB | A | 143 | 7.7 | −7.0 | 11.7 | 37 |
| THR | OG1 | A | 143 | 6.7 | −6.1 | 12.1 | 37 |
| THR | CG2 | A | 143 | 7.1 | −8.1 | 10.9 | 35 |
| THR | C | A | 143 | 9.3 | −6.4 | 13.5 | 39 |
| THR | O | A | 143 | 10.3 | −5.9 | 12.9 | 42 |
| GLU | N | A | 144 | 8.9 | −5.9 | 14.7 | 38 |
| GLU | CA | A | 144 | 9.6 | −4.9 | 15.4 | 42 |
| GLU | CB | A | 144 | 9.2 | −3.5 | 15.0 | 50 |
| GLU | CG | A | 144 | 9.5 | −3.2 | 13.5 | 59 |
| GLU | CD | A | 144 | 9.0 | −1.8 | 13.2 | 67 |
| GLU | OE1 | A | 144 | 8.6 | −1.0 | 14.1 | 69 |
| GLU | OE2 | A | 144 | 9.1 | −1.4 | 12.0 | 68 |
| GLU | C | A | 144 | 9.4 | −5.0 | 16.9 | 39 |
| GLU | O | A | 144 | 8.3 | −5.2 | 17.4 | 37 |
| SER | N | A | 145 | 10.5 | −5.0 | 17.7 | 35 |
| SER | CA | A | 145 | 10.4 | −5.2 | 19.1 | 34 |
| SER | CB | A | 145 | 10.6 | −6.6 | 19.5 | 27 |
| SER | OG | A | 145 | 11.9 | −7.1 | 19.1 | 35 |
| SER | C | A | 145 | 11.4 | −4.3 | 19.9 | 33 |
| SER | O | A | 145 | 12.5 | −4.0 | 19.4 | 31 |
| ASP | N | A | 146 | 11.0 | −3.9 | 21.1 | 35 |
| ASP | CA | A | 146 | 11.9 | −3.1 | 21.9 | 35 |
| ASP | CB | A | 146 | 11.4 | −1.6 | 21.9 | 44 |
| ASP | CG | A | 146 | 12.3 | −0.7 | 22.8 | 51 |
| ASP | OD1 | A | 146 | 11.8 | −0.2 | 23.8 | 56 |
| ASP | OD2 | A | 146 | 13.5 | −0.6 | 22.5 | 54 |
| ASP | C | A | 146 | 11.9 | −3.6 | 23.4 | 33 |
| ASP | O | A | 146 | 10.8 | −3.7 | 24.0 | 28 |
| LYS | N | A | 147 | 13.0 | −4.1 | 23.8 | 32 |
| LYS | CA | A | 147 | 13.2 | −4.6 | 25.2 | 35 |
| LYS | CB | A | 147 | 12.9 | −3.5 | 26.2 | 39 |
| LYS | CG | A | 147 | 13.9 | −2.3 | 26.1 | 45 |
| LYS | CD | A | 147 | 13.6 | −1.2 | 27.1 | 52 |
| LYS | CE | A | 147 | 14.6 | −0.1 | 26.9 | 55 |
| LYS | NZ | A | 147 | 14.3 | 1.0 | 27.9 | 59 |
| LYS | C | A | 147 | 12.3 | −5.8 | 25.5 | 34 |
| LYS | O | A | 147 | 12.1 | −6.1 | 26.6 | 36 |
| PHE | N | A | 148 | 11.8 | −6.4 | 24.4 | 31 |
| PHE | CA | A | 148 | 10.9 | −7.6 | 24.5 | 27 |
| PHE | CB | A | 148 | 10.0 | −7.7 | 23.3 | 24 |
| PHE | CG | A | 148 | 9.0 | −8.8 | 23.4 | 25 |
| PHE | CD1 | A | 148 | 8.1 | −9.0 | 24.4 | 27 |
| PHE | CD2 | A | 148 | 9.1 | −9.8 | 22.4 | 23 |
| PHE | CE1 | A | 148 | 7.2 | −10.0 | 24.5 | 25 |
| PHE | CE2 | A | 148 | 8.2 | −10.9 | 22.4 | 19 |
| PHE | CZ | A | 148 | 7.2 | −11.0 | 23.5 | 22 |
| PHE | C | A | 148 | 11.8 | −8.8 | 24.6 | 27 |
| PHE | O | A | 148 | 11.9 | −9.5 | 25.7 | 27 |
| PHE | N | A | 149 | 12.4 | −9.2 | 23.5 | 24 |
| PHE | CA | A | 149 | 13.3 | −10.4 | 23.4 | 28 |
| PHE | CB | A | 149 | 13.7 | −10.7 | 22.0 | 25 |
| PHE | CG | A | 149 | 12.6 | −11.0 | 21.1 | 24 |
| PHE | CD1 | A | 149 | 12.3 | −10.1 | 20.0 | 26 |
| PHE | CD2 | A | 149 | 11.8 | −12.1 | 21.2 | 24 |
| PHE | CE1 | A | 149 | 11.3 | −10.4 | 19.1 | 27 |
| PHE | CE2 | A | 149 | 10.8 | −12.4 | 20.4 | 23 |
| PHE | CZ | A | 149 | 10.5 | −11.5 | 19.3 | 23 |
| PHE | C | A | 149 | 14.5 | −10.2 | 24.4 | 33 |
| PHE | O | A | 149 | 15.2 | −9.2 | 24.4 | 34 |
| ILE | N | A | 150 | 14.8 | −11.3 | 25.1 | 38 |
| ILE | CA | A | 150 | 15.9 | −11.3 | 26.0 | 37 |
| ILE | CB | A | 150 | 15.5 | −11.9 | 27.4 | 37 |
| ILE | CG2 | A | 150 | 16.7 | −12.0 | 28.3 | 37 |
| ILE | CG1 | A | 150 | 14.4 | −11.0 | 28.0 | 35 |
| ILE | CD1 | A | 150 | 14.0 | −11.5 | 29.4 | 31 |
| ILE | C | A | 150 | 17.1 | −12.1 | 25.5 | 38 |
| ILE | O | A | 150 | 16.9 | −13.2 | 24.9 | 37 |
| ASN | N | A | 151 | 18.3 | −11.5 | 25.6 | 39 |
| ASN | CA | A | 151 | 19.5 | −12.2 | 25.1 | 41 |
| ASN | CB | A | 151 | 20.7 | −11.2 | 25.0 | 46 |
| ASN | CG | A | 151 | 21.9 | −11.8 | 24.4 | 50 |
| ASN | OD1 | A | 151 | 23.0 | −11.7 | 25.0 | 54 |
| ASN | ND2 | A | 151 | 21.8 | −12.4 | 23.2 | 52 |
| ASN | C | A | 151 | 19.8 | −13.5 | 25.9 | 40 |
| ASN | O | A | 151 | 20.1 | −13.4 | 27.1 | 35 |
| GLY | N | A | 152 | 19.8 | −14.6 | 25.2 | 39 |
| GLY | CA | A | 152 | 20.1 | −15.9 | 25.8 | 37 |
| GLY | C | A | 152 | 18.9 | −16.6 | 26.5 | 39 |
| GLY | O | A | 152 | 19.1 | −17.6 | 27.1 | 41 |
| SER | N | A | 153 | 17.7 | −16.0 | 26.4 | 36 |
| SER | CA | A | 153 | 16.6 | −16.7 | 27.1 | 35 |
| SER | CB | A | 153 | 15.4 | −15.7 | 27.2 | 29 |
| SER | OG | A | 153 | 15.0 | −15.4 | 25.9 | 23 |
| SER | C | A | 153 | 16.2 | −18.0 | 26.4 | 34 |
| SER | O | A | 153 | 15.5 | −18.8 | 27.0 | 35 |
| ASN | N | A | 154 | 16.6 | −18.2 | 25.2 | 37 |
| ASN | CA | A | 154 | 16.3 | −19.3 | 24.4 | 37 |
| ASN | CB | A | 154 | 16.8 | −20.6 | 25.1 | 37 |
| ASN | CG | A | 154 | 16.7 | −21.8 | 24.2 | 38 |
| ASN | OD1 | A | 154 | 16.6 | −21.7 | 22.9 | 33 |
| ASN | ND2 | A | 154 | 16.7 | −23.0 | 24.8 | 36 |
| ASN | C | A | 154 | 14.9 | −19.4 | 23.9 | 36 |
| ASN | O | A | 154 | 14.4 | −20.4 | 23.3 | 38 |
| TRP | N | A | 155 | 14.1 | −18.4 | 24.1 | 31 |
| TRP | CA | A | 155 | 12.7 | −18.4 | 23.6 | 31 |
| TRP | CB | A | 155 | 11.6 | −18.2 | 24.7 | 27 |
| TRP | CG | A | 155 | 11.7 | −17.0 | 25.6 | 26 |
| TRP | CD2 | A | 155 | 11.2 | −15.7 | 25.4 | 24 |
| TRP | CE2 | A | 155 | 11.5 | −15.0 | 26.5 | 23 |
| TRP | CE3 | A | 155 | 10.6 | −15.1 | 24.3 | 27 |
| TRP | CD1 | A | 155 | 12.3 | −17.0 | 26.9 | 25 |
| TRP | NE1 | A | 155 | 12.1 | −15.8 | 27.4 | 24 |
| TRP | CZ2 | A | 155 | 11.2 | −13.6 | 26.6 | 23 |
| TRP | CZ3 | A | 155 | 10.2 | −13.8 | 24.4 | 28 |
| TRP | CH2 | A | 155 | 10.5 | −13.0 | 25.6 | 25 |
| TRP | C | A | 155 | 12.5 | −17.3 | 22.5 | 30 |
| TRP | O | A | 155 | 13.0 | −16.2 | 22.5 | 28 |
| GLU | N | A | 156 | 11.7 | −17.7 | 21.4 | 32 |
| GLU | CA | A | 156 | 11.5 | −16.8 | 20.3 | 32 |
| GLU | CB | A | 156 | 11.7 | −17.5 | 18.9 | 30 |
| GLU | CG | A | 156 | 13.1 | −18.1 | 18.7 | 36 |
| GLU | CD | A | 156 | 13.4 | −19.3 | 19.6 | 35 |
| GLU | OE1 | A | 156 | 14.4 | −19.2 | 20.3 | 40 |
| GLU | OE2 | A | 156 | 12.6 | −20.2 | 19.7 | 36 |
| GLU | C | A | 156 | 10.1 | −16.2 | 20.2 | 30 |
| GLU | O | A | 156 | 9.8 | −15.3 | 19.4 | 32 |
| GLY | N | A | 157 | 9.2 | −16.6 | 21.1 | 32 |
| GLY | CA | A | 157 | 7.8 | −16.1 | 21.1 | 27 |
| CLY | C | A | 157 | 7.1 | −16.1 | 22.4 | 28 |
| GLY | O | A | 157 | 7.7 | −16.3 | 23.5 | 29 |
| ILE | N | A | 158 | 5.8 | −15.8 | 22.4 | 28 |
| ILE | CA | A | 158 | 5.0 | −15.7 | 23.6 | 24 |
| ILE | CB | A | 158 | 5.0 | −14.2 | 24.2 | 22 |
| ILE | CG2 | A | 158 | 4.4 | −13.3 | 23.1 | 21 |
| ILE | CG1 | A | 158 | 4.3 | −14.1 | 25.5 | 18 |
| ILE | CD1 | A | 158 | 4.3 | −12.7 | 26.1 | 23 |
| ILE | C | A | 158 | 3.6 | −16.2 | 23.5 | 25 |
| ILE | O | A | 158 | 2.9 | −15.8 | 22.6 | 26 |
| LEU | N | A | 159 | 3.2 | −17.0 | 24.5 | 27 |
| LEU | CA | A | 159 | 1.8 | −17.5 | 24.6 | 28 |
| LEU | CB | A | 159 | 1.9 | −19.0 | 24.9 | 25 |
| LEU | CG | A | 159 | 0.5 | −19.7 | 25.0 | 27 |
| LEU | CD1 | A | 159 | −0.3 | −19.5 | 23.7 | 30 |
| LEU | CD2 | A | 159 | 0.6 | −21.2 | 25.3 | 24 |
| LEU | C | A | 159 | 1.0 | −16.8 | 25.6 | 28 |
| LEU | O | A | 159 | 1.1 | −16.9 | 26.8 | 28 |
| GLY | N | A | 160 | 0.1 | −15.9 | 25.1 | 29 |
| GLY | CA | A | 160 | −0.8 | −15.2 | 26.0 | 29 |
| GLY | C | A | 160 | −2.0 | −16.0 | 26.4 | 31 |
| GLY | O | A | 160 | −2.9 | −16.3 | 25.6 | 29 |
| LEU | N | A | 161 | −2.0 | −16.4 | 27.7 | 32 |
| LEU | CA | A | 161 | −3.0 | −17.3 | 28.2 | 30 |
| LEU | CB | A | 161 | −2.4 | −18.3 | 29.2 | 31 |
| LEU | CG | A | 161 | −1.4 | −19.3 | 28.5 | 34 |
| LEU | CD1 | A | 161 | −0.8 | −20.2 | 29.5 | 32 |
| LEU | CD2 | A | 161 | −2.1 | −20.1 | 27.4 | 35 |
| LEU | C | A | 161 | −4.2 | −16.6 | 28.8 | 30 |
| LEU | O | A | 161 | −5.2 | −17.2 | 29.3 | 26 |
| ALA | N | A | 162 | −4.2 | −15.3 | 28.9 | 27 |
| ALA | CA | A | 162 | −5.3 | −14.5 | 29.5 | 29 |
| ALA | CB | A | 162 | −4.9 | −13.1 | 29.9 | 29 |
| ALA | C | A | 162 | −6.5 | −14.5 | 28.6 | 31 |
| ALA | O | A | 162 | −6.5 | −15.1 | 27.5 | 29 |
| TYR | N | A | 163 | −7.5 | −13.7 | 29.0 | 32 |
| TYR | CA | A | 163 | −8.8 | −13.6 | 28.2 | 32 |
| TYR | CB | A | 163 | −10.0 | −13.4 | 29.1 | 32 |
| TYR | CG | A | 163 | −10.1 | −14.5 | 30.1 | 30 |
| TYR | CD1 | A | 163 | −9.5 | −14.4 | 31.4 | 31 |
| TYR | CE1 | A | 163 | −9.6 | −15.4 | 32.4 | 30 |
| TYR | CD2 | A | 163 | −10.8 | −15.7 | 29.8 | 27 |
| TYR | CE2 | A | 163 | −10.9 | −16.7 | 30.8 | 28 |
| TYR | CZ | A | 163 | −10.3 | −16.5 | 32.0 | 25 |
| TYR | OH | A | 163 | −10.4 | −17.5 | 33.0 | 28 |
| TYR | C | A | 163 | −8.8 | −12.6 | 27.0 | 33 |
| TYR | O | A | 163 | −8.1 | −11.6 | 27.1 | 30 |
| ALA | N | A | 164 | −9.6 | −12.8 | 26.1 | 34 |
| ALA | CA | A | 164 | −9.8 | −12.0 | 24.9 | 36 |
| ALA | CB | A | 164 | −10.9 | −12.6 | 24.0 | 37 |
| ALA | C | A | 164 | −10.1 | −10.5 | 25.2 | 35 |
| ALA | O | A | 164 | −10.0 | −9.7 | 24.2 | 41 |
| GLU | N | A | 165 | −10.4 | −10.2 | 26.4 | 37 |
| GLU | CA | A | 165 | −10.8 | −8.8 | 26.7 | 35 |
| GLU | CB | A | 165 | −11.6 | −8.6 | 28.0 | 42 |
| GLU | CG | A | 165 | −11.9 | −7.2 | 28.3 | 49 |
| GLU | CD | A | 165 | −12.7 | −7.1 | 29.6 | 55 |
| GLU | OE1 | A | 165 | −13.0 | −8.1 | 30.2 | 56 |
| GLU | OE2 | A | 165 | −13.0 | −6.0 | 30.0 | 56 |
| GLU | C | A | 165 | −9.5 | −7.9 | 26.7 | 33 |
| GLU | O | A | 165 | −9.6 | −6.7 | 26.5 | 30 |
| ILE | N | A | 166 | −8.3 | −8.4 | 26.9 | 34 |
| ILE | CA | A | 166 | −7.1 | −7.6 | 26.9 | 31 |
| ILE | CB | A | 166 | −6.3 | −7.8 | 28.2 | 31 |
| ILE | CG2 | A | 166 | −7.2 | −7.2 | 29.4 | 28 |
| ILE | CG1 | A | 166 | −5.9 | −9.2 | 28.5 | 28 |
| ILE | CD1 | A | 166 | −5.1 | −9.5 | 29.8 | 27 |
| ILE | C | A | 166 | −6.2 | −7.9 | 25.7 | 31 |
| ILE | O | A | 166 | −5.1 | −7.4 | 25.7 | 32 |
| ALA | N | A | 167 | −6.7 | −8.7 | 24.8 | 33 |
| ALA | CA | A | 167 | −6.0 | −9.0 | 23.6 | 33 |
| ALA | CB | A | 167 | −6.5 | −10.2 | 22.9 | 28 |
| ALA | C | A | 167 | −6.0 | −7.8 | 22.6 | 31 |
| ALA | O | A | 167 | −7.0 | −7.1 | 22.5 | 34 |
| ARG | N | A | 168 | −4.9 | −7.6 | 21.9 | 34 |
| ARG | CA | A | 168 | −4.8 | −6.5 | 20.9 | 36 |
| ARG | CB | A | 168 | −3.5 | −5.8 | 20.9 | 38 |
| ARG | CG | A | 168 | −3.2 | −5.0 | 22.3 | 42 |
| ARG | CD | A | 168 | −4.3 | −4.0 | 22.6 | 44 |
| ARG | NE | A | 168 | −4.0 | −3.3 | 23.8 | 51 |
| ARG | CZ | A | 168 | −3.0 | −2.5 | 24.0 | 53 |
| ARG | NH1 | A | 168 | −2.1 | −2.3 | 23.0 | 56 |
| ARG | NH2 | A | 168 | −2.8 | −1.9 | 25.2 | 51 |
| ARG | C | A | 168 | −5.0 | −7.1 | 19.5 | 41 |
| ARG | O | A | 168 | −4.6 | −8.3 | 19.3 | 36 |
| PRO | N | A | 169 | −5.5 | −6.4 | 18.5 | 42 |
| PRO | CD | A | 169 | −5.2 | −6.7 | 17.1 | 46 |
| PRO | CA | A | 169 | −6.0 | −5.0 | 18.6 | 46 |
| PRO | CB | A | 169 | −6.3 | −4.7 | 17.1 | 49 |
| PRO | CG | A | 169 | −5.1 | −5.3 | 16.5 | 50 |
| PRO | C | A | 169 | −7.2 | −4.8 | 19.5 | 46 |
| PRO | O | A | 169 | −7.4 | −3.7 | 20.1 | 47 |
| ASP | N | A | 170 | −8.0 | −5.9 | 19.6 | 47 |
| ASP | CA | A | 170 | −9.2 | −5.8 | 20.5 | 49 |
| ASP | CB | A | 170 | −10.2 | −4.9 | 19.8 | 53 |
| ASP | CG | A | 170 | −10.7 | −5.4 | 18.5 | 59 |
| ASP | OD1 | A | 170 | −11.8 | −5.8 | 18.3 | 60 |
| ASP | OD2 | A | 170 | −9.9 | −5.4 | 17.6 | 64 |
| ASP | C | A | 170 | −9.7 | −7.2 | 20.8 | 48 |
| ASP | O | A | 170 | −9.2 | −8.2 | 20.2 | 45 |
| ASP | N | A | 171 | −10.7 | −7.3 | 21.7 | 48 |
| ASP | CA | A | 171 | −11.3 | −8.5 | 22.1 | 48 |
| ASP | CB | A | 171 | −12.4 | −8.2 | 23.1 | 52 |
| ASP | CG | A | 171 | −13.5 | −7.4 | 22.5 | 56 |
| ASP | OD1 | A | 171 | −13.7 | −6.2 | 23.0 | 57 |
| ASP | OD2 | A | 171 | −14.2 | −7.8 | 21.6 | 59 |
| ASP | C | A | 171 | −11.8 | −9.5 | 21.0 | 49 |
| ASP | O | A | 171 | −12.2 | −10.6 | 21.3 | 50 |
| SER | N | A | 172 | −11.7 | −9.0 | 19.8 | 49 |
| SER | CA | A | 172 | −12.2 | −9.9 | 18.7 | 48 |
| SER | CB | A | 172 | −12.7 | −9.1 | 17.5 | 50 |
| SER | OG | A | 172 | −11.6 | −8.2 | 17.0 | 54 |
| SER | C | A | 172 | −11.1 | −10.9 | 18.2 | 48 |
| SER | O | A | 172 | −11.4 | −11.9 | 17.5 | 45 |
| LEU | N | A | 173 | −9.8 | −10.6 | 18.6 | 44 |
| LEU | CA | A | 173 | −8.7 | −11.5 | 18.2 | 41 |
| LEU | CB | A | 173 | −7.4 | −10.7 | 18.2 | 43 |
| LEU | CG | A | 173 | −6.2 | −11.6 | 17.7 | 43 |
| LEU | CD1 | A | 173 | −6.4 | −12.0 | 16.3 | 43 |
| LEU | CD2 | A | 173 | −4.9 | −10.8 | 17.9 | 39 |
| LEU | C | A | 173 | −8.7 | −12.7 | 19.2 | 40 |
| LEU | O | A | 173 | −8.2 | −12.6 | 20.3 | 37 |
| GLU | N | A | 174 | −9.3 | −13.8 | 18.7 | 40 |
| GLU | CA | A | 174 | −9.4 | −15.0 | 19.6 | 40 |
| GLU | CB | A | 174 | −10.3 | −16.1 | 18.9 | 40 |
| GLU | CG | A | 174 | −10.4 | −17.3 | 19.7 | 43 |
| GLU | CD | A | 174 | −11.3 | −18.3 | 18.9 | 45 |
| GLU | OE1 | A | 174 | −12.4 | −18.5 | 19.3 | 45 |
| GLU | OE2 | A | 174 | −10.8 | −18.9 | 18.0 | 45 |
| GLU | C | A | 174 | −8.1 | −15.5 | 20.1 | 41 |
| GLU | O | A | 174 | −7.2 | −15.9 | 19.3 | 37 |
| PRO | N | A | 175 | −7.9 | −15.6 | 21.4 | 38 |
| PRO | CD | A | 175 | −8.7 | −14.7 | 22.3 | 38 |
| PRO | CA | A | 175 | −6.8 | −16.1 | 22.1 | 33 |
| PRO | CB | A | 175 | −7.2 | −15.9 | 23.6 | 37 |
| PRO | CG | A | 175 | −7.7 | −14.5 | 23.5 | 40 |
| PRO | C | A | 175 | −6.5 | −17.5 | 21.8 | 32 |
| PRO | O | A | 175 | −7.4 | −18.3 | 21.5 | 28 |
| PHE | N | A | 176 | −5.2 | −17.9 | 21.8 | 28 |
| PHE | CA | A | 176 | −4.8 | −19.3 | 21.5 | 28 |
| PHE | CB | A | 176 | −3.3 | −19.5 | 21.6 | 29 |
| PHE | CG | A | 176 | −2.8 | −20.9 | 21.4 | 29 |
| PHE | CD1 | A | 176 | −2.6 | −21.4 | 20.1 | 30 |
| PHE | CD2 | A | 176 | −2.7 | −21.7 | 22.5 | 28 |
| PHE | CE1 | A | 176 | −2.3 | −22.8 | 19.9 | 28 |
| PHE | CE2 | A | 176 | −2.3 | −23.1 | 22.3 | 28 |
| PHE | CZ | A | 176 | −2.1 | −23.6 | 21.0 | 29 |
| PHE | C | A | 176 | −5.4 | −20.4 | 22.3 | 32 |
| PHE | O | A | 176 | −5.6 | −21.5 | 21.8 | 33 |
| PHE | N | A | 177 | −5.8 | −20.1 | 23.5 | 34 |
| PHE | CA | A | 177 | −6.4 | −21.2 | 24.3 | 34 |
| PHE | CB | A | 177 | −6.1 | −21.0 | 25.8 | 32 |
| PHE | CG | A | 177 | −6.4 | −22.2 | 26.7 | 31 |
| PHE | CD1 | A | 177 | −5.5 | −23.2 | 26.9 | 26 |
| PHE | CD2 | A | 177 | −7.7 | −22.4 | 27.2 | 30 |
| PHE | CE1 | A | 177 | −5.7 | −24.3 | 27.7 | 29 |
| PHE | CE2 | A | 177 | −8.0 | −23.5 | 28.0 | 30 |
| PHE | CZ | A | 177 | −7.0 | −24.5 | 28.3 | 28 |
| PHE | C | A | 177 | −7.9 | −21.4 | 24.0 | 36 |
| PHE | O | A | 177 | −8.4 | −22.5 | 24.1 | 27 |
| ASP | N | A | 178 | −8.5 | −20.3 | 23.7 | 36 |
| ASP | CA | A | 178 | −10.0 | −20.4 | 23.3 | 41 |
| ASP | CB | A | 178 | −10.6 | −19.0 | 23.2 | 45 |
| ASP | CG | A | 178 | −10.5 | −18.2 | 24.4 | 50 |
| ASP | OD1 | A | 178 | −11.5 | −17.8 | 25.0 | 49 |
| ASP | OD2 | A | 178 | −9.4 | −17.9 | 24.9 | 52 |
| ASP | C | A | 178 | −10.1 | −21.2 | 22.0 | 39 |
| ASP | O | A | 178 | −11.1 | −21.9 | 21.9 | 40 |
| SER | N | A | 179 | −9.1 | −21.0 | 21.1 | 38 |
| SER | CA | A | 179 | −9.2 | −21.7 | 19.9 | 37 |
| SER | CB | A | 179 | −8.2 | −21.2 | 18.9 | 34 |
| SER | OG | A | 179 | −8.4 | −19.8 | 18.6 | 38 |
| SER | C | A | 179 | −8.8 | −23.2 | 20.1 | 39 |
| SER | O | A | 179 | −9.3 | −24.1 | 19.4 | 42 |
| LEU | N | A | 180 | −8.0 | −23.4 | 21.2 | 39 |
| LEU | CA | A | 180 | −7.6 | −24.7 | 21.5 | 39 |
| LEU | CB | A | 180 | −6.6 | −24.7 | 22.6 | 38 |
| LEU | CG | A | 180 | −6.1 | −26.1 | 23.1 | 37 |
| LEU | CD1 | A | 180 | −5.6 | −26.9 | 21.9 | 35 |
| LEU | CD2 | A | 180 | −5.0 | −26.0 | 24.2 | 36 |
| LEU | C | A | 180 | −8.9 | −25.5 | 22.0 | 37 |
| LEU | O | A | 180 | −9.0 | −26.7 | 21.6 | 38 |
| VAL | N | A | 181 | −9.7 | −24.9 | 22.8 | 34 |
| VAL | CA | A | 181 | −10.9 | −25.5 | 23.3 | 36 |
| VAL | CB | A | 181 | −11.4 | −24.8 | 24.6 | 36 |
| VAL | CG1 | A | 181 | −12.7 | −25.5 | 25.1 | 32 |
| VAL | CG2 | A | 181 | −10.4 | −24.7 | 25.7 | 35 |
| VAL | C | A | 181 | −12.0 | −25.6 | 22.3 | 38 |
| VAL | O | A | 181 | −12.7 | −26.7 | 22.2 | 34 |
| LYS | N | A | 182 | −12.1 | −24.6 | 21.4 | 41 |
| LYS | CA | A | 182 | −13.2 | −24.7 | 20.4 | 44 |
| LYS | CB | A | 182 | −13.5 | −23.3 | 19.9 | 44 |
| LYS | CG | A | 182 | −14.1 | −22.3 | 20.9 | 44 |
| LYS | CD | A | 182 | −14.3 | −21.0 | 20.1 | 49 |
| LYS | CE | A | 182 | −14.9 | −19.9 | 21.1 | 53 |
| LYS | NZ | A | 182 | −16.2 | −20.2 | 21.7 | 58 |
| LYS | C | A | 182 | −12.9 | −25.6 | 19.2 | 44 |
| LYS | O | A | 182 | −13.7 | −25.8 | 18.4 | 49 |
| GLN | N | A | 183 | −11.7 | −26.2 | 19.2 | 41 |
| GLN | CA | A | 183 | −11.4 | −27.0 | 18.1 | 36 |
| GLN | CB | A | 183 | −10.3 | −26.4 | 17.2 | 36 |
| GLN | CG | A | 183 | −10.6 | −25.0 | 16.7 | 35 |
| GLN | CD | A | 183 | −9.4 | −24.5 | 15.9 | 35 |
| GLN | OE1 | A | 183 | −8.5 | −25.2 | 15.6 | 36 |
| GLN | NE2 | A | 183 | −9.4 | −23.2 | 15.7 | 37 |
| GLN | C | A | 183 | −10.9 | −28.4 | 18.5 | 38 |
| GLN | O | A | 183 | −10.8 | −29.3 | 17.6 | 32 |
| THR | N | A | 184 | −10.7 | −28.6 | 19.8 | 36 |
| THR | CA | A | 184 | −10.3 | −29.9 | 20.3 | 37 |
| THR | CB | A | 184 | −8.8 | −30.0 | 20.6 | 39 |
| THR | OG1 | A | 184 | −8.5 | −29.1 | 21.6 | 39 |
| THR | CG2 | A | 184 | −8.0 | −29.6 | 19.3 | 40 |
| THR | C | A | 184 | −11.0 | −30.4 | 21.5 | 36 |
| THR | O | A | 184 | −11.9 | −29.7 | 22.0 | 37 |
| HIS | N | A | 185 | −10.6 | −31.6 | 22.0 | 40 |
| HIS | CA | A | 185 | −11.2 | −32.2 | 23.2 | 45 |
| HIS | CB | A | 185 | −11.0 | −33.7 | 23.1 | 47 |
| HIS | CG | A | 185 | −9.6 | −34.1 | 23.1 | 52 |
| HIS | CD2 | A | 185 | −8.8 | −34.5 | 24.1 | 53 |
| HIS | ND1 | A | 185 | −8.8 | −34.0 | 22.0 | 55 |
| HIS | CE1 | A | 185 | −7.5 | −34.4 | 22.3 | 54 |
| HIS | NE2 | A | 185 | −7.5 | −34.7 | 23.6 | 55 |
| HIS | C | A | 185 | −10.7 | −31.6 | 24.5 | 43 |
| HIS | O | A | 185 | −11.1 | −32.0 | 25.5 | 44 |
| VAL | N | A | 186 | −9.6 | −30.8 | 24.3 | 38 |
| VAL | CA | A | 186 | −9.0 | −30.2 | 25.5 | 33 |
| VAL | CB | A | 186 | −7.7 | −29.4 | 25.1 | 34 |
| VAL | CG1 | A | 186 | −7.0 | −28.8 | 26.4 | 35 |
| VAL | CG2 | A | 186 | −6.7 | −30.2 | 24.3 | 32 |
| VAL | C | A | 186 | −9.9 | −29.4 | 26.4 | 27 |
| VAL | O | A | 186 | −10.5 | −28.4 | 26.0 | 30 |
| PRO | N | A | 187 | −10.1 | −29.9 | 27.7 | 27 |
| PRO | CD | A | 187 | −9.7 | −31.2 | 28.1 | 30 |
| PRO | CA | A | 187 | −10.9 | −29.2 | 28.7 | 28 |
| PRO | CB | A | 187 | −10.6 | −30.1 | 29.9 | 27 |
| PRO | CG | A | 187 | −10.7 | −31.4 | 29.3 | 28 |
| PRO | C | A | 187 | −10.5 | −27.8 | 28.9 | 29 |
| PRO | O | A | 187 | −9.3 | −27.5 | 28.7 | 28 |
| ASN | N | A | 188 | −11.4 | −26.9 | 29.2 | 31 |
| ASN | CA | A | 188 | −11.1 | −25.5 | 29.3 | 31 |
| ASN | CB | A | 188 | −12.4 | −24.6 | 29.2 | 28 |
| ASN | CG | A | 188 | −12.1 | −23.2 | 29.3 | 31 |
| ASN | OD1 | A | 188 | −11.0 | −22.7 | 29.1 | 32 |
| ASN | ND2 | A | 188 | −13.1 | −22.4 | 29.5 | 27 |
| ASN | C | A | 188 | −10.5 | −25.3 | 30.7 | 34 |
| ASN | O | A | 188 | −11.1 | −24.8 | 31.6 | 30 |
| LEU | N | A | 189 | −9.2 | −25.7 | 30.8 | 35 |
| LEU | CA | A | 189 | −8.5 | −25.7 | 32.1 | 36 |
| LEU | CB | A | 189 | −9.2 | −26.6 | 33.1 | 33 |
| LEU | CG | A | 189 | −8.6 | −26.7 | 34.5 | 36 |
| LEU | CD1 | A | 189 | −9.5 | −27.5 | 35.4 | 33 |
| LEU | CD2 | A | 189 | −7.1 | −27.3 | 34.5 | 32 |
| LEU | C | A | 189 | −7.0 | −26.0 | 31.9 | 35 |
| LEU | O | A | 189 | −6.7 | −26.9 | 31.1 | 35 |
| PHE | N | A | 190 | −6.1 | −25.3 | 32.5 | 31 |
| PHE | CA | A | 190 | −4.7 | −25.6 | 32.4 | 31 |
| PHE | CB | A | 190 | −3.9 | −24.8 | 31.3 | 27 |
| PHE | CG | A | 190 | −3.9 | −23.3 | 31.6 | 30 |
| PHE | CD1 | A | 190 | −5.0 | −22.5 | 31.2 | 32 |
| PHE | CD2 | A | 190 | −2.8 | −22.8 | 32.1 | 26 |
| PHE | CE1 | A | 190 | −4.9 | −21.1 | 31.4 | 33 |
| PHE | CE2 | A | 190 | −2.7 | −21.4 | 32.3 | 28 |
| PHE | CZ | A | 190 | −3.8 | −20.6 | 32.0 | 29 |
| PHE | C | A | 190 | −4.1 | −25.4 | 33.8 | 28 |
| PHE | O | A | 190 | −4.6 | −24.6 | 34.6 | 23 |
| SER | N | A | 191 | −2.9 | −26.1 | 34.0 | 26 |
| SER | CA | A | 191 | −2.2 | −25.9 | 35.3 | 25 |
| SER | CB | A | 191 | −2.6 | −27.1 | 36.2 | 26 |
| SER | OG | A | 191 | −2.2 | −28.4 | 35.6 | 25 |
| SER | C | A | 191 | −0.7 | −25.7 | 35.1 | 26 |
| SER | O | A | 191 | −0.1 | −26.3 | 34.2 | 24 |
| LEU | N | A | 192 | −0.2 | −24.9 | 36.0 | 25 |
| LEU | CA | A | 192 | 1.2 | −24.6 | 36.0 | 26 |
| LEU | CB | A | 192 | 1.5 | −23.1 | 35.7 | 20 |
| LEU | CG | A | 192 | 1.0 | −22.7 | 34.3 | 22 |
| LEU | CD1 | A | 192 | 1.1 | −21.2 | 34.1 | 17 |
| LEU | CD2 | A | 192 | 1.7 | −23.5 | 33.2 | 21 |
| LEU | C | A | 192 | 2.1 | −24.9 | 37.3 | 26 |
| LEU | O | A | 192 | 1.7 | −24.4 | 38.4 | 25 |
| GLN | N | A | 193 | 3.1 | −25.6 | 37.1 | 26 |
| GLN | CA | A | 193 | 4.1 | −25.9 | 38.2 | 25 |
| GLN | CB | A | 193 | 4.3 | −27.4 | 38.4 | 27 |
| GLN | CG | A | 193 | 5.3 | −27.6 | 39.5 | 30 |
| GLN | CD | A | 193 | 5.6 | −29.1 | 39.8 | 34 |
| GLN | OE1 | A | 193 | 6.4 | −29.7 | 39.1 | 36 |
| GLN | NE2 | A | 193 | 4.9 | −29.7 | 40.8 | 34 |
| GLN | C | A | 193 | 5.4 | −25.3 | 37.8 | 27 |
| GLN | O | A | 193 | 6.1 | −25.9 | 37.0 | 30 |
| LEU | N | A | 194 | 5.7 | −24.1 | 38.4 | 29 |
| LEU | CA | A | 194 | 7.0 | −23.5 | 38.0 | 28 |
| LEU | CB | A | 194 | 6.8 | −22.0 | 38.0 | 24 |
| LEU | CG | A | 194 | 5.8 | −21.6 | 36.9 | 26 |
| LEU | CD1 | A | 194 | 5.6 | −20.1 | 36.9 | 24 |
| LEU | CD2 | A | 194 | 6.2 | −22.1 | 35.5 | 26 |
| LEU | C | A | 194 | 8.0 | −23.9 | 39.1 | 27 |
| LEU | O | A | 194 | 7.7 | −23.7 | 40.3 | 26 |
| CYS | N | A | 195 | 9.1 | −24.5 | 38.8 | 28 |
| CYS | CA | A | 195 | 10.0 | −25.0 | 39.8 | 39 |
| CYS | C | A | 195 | 11.3 | −24.2 | 40.1 | 45 |
| CYS | O | A | 195 | 12.0 | −24.5 | 41.1 | 52 |
| CYS | CB | A | 195 | 10.5 | −26.4 | 39.5 | 39 |
| CYS | SG | A | 195 | 9.1 | −27.6 | 39.4 | 39 |
| GLY | N | A | 196 | 11.5 | −23.1 | 39.4 | 50 |
| GLY | CA | A | 196 | 12.7 | −22.3 | 39.6 | 57 |
| GLY | C | A | 196 | 14.0 | −23.2 | 39.6 | 60 |
| GLY | O | A | 196 | 14.2 | −23.9 | 38.7 | 60 |
| ALA | N | A | 197 | 14.7 | −23.2 | 40.7 | 59 |
| ALA | CA | A | 197 | 15.9 | −24.0 | 40.9 | 61 |
| ALA | CB | A | 197 | 17.1 | −23.3 | 40.2 | 62 |
| ALA | C | A | 197 | 16.2 | −24.4 | 42.3 | 61 |
| ALA | O | A | 197 | 16.4 | −25.5 | 42.7 | 62 |
| ALA | N | A | 208 | 21.6 | −22.0 | 37.0 | 75 |
| ALA | CA | A | 208 | 21.0 | −20.9 | 36.3 | 74 |
| ALA | CB | A | 208 | 22.0 | −20.2 | 35.4 | 71 |
| ALA | C | A | 208 | 19.8 | −21.3 | 35.5 | 74 |
| ALA | O | A | 208 | 19.1 | −20.5 | 34.9 | 75 |
| SER | N | A | 209 | 19.5 | −22.6 | 35.5 | 71 |
| SER | CA | A | 209 | 18.4 | −23.2 | 34.8 | 68 |
| SER | CB | A | 209 | 18.9 | −24.4 | 34.0 | 69 |
| SER | OG | A | 209 | 19.9 | −24.1 | 33.1 | 69 |
| SER | C | A | 209 | 17.2 | −23.5 | 35.6 | 66 |
| SER | O | A | 209 | 17.3 | −24.1 | 36.7 | 65 |
| VAL | N | A | 210 | 16.0 | −23.2 | 35.1 | 62 |
| VAL | CA | A | 210 | 14.7 | −23.4 | 35.7 | 57 |
| VAL | CB | A | 210 | 14.0 | −22.1 | 36.0 | 56 |
| VAL | CG1 | A | 210 | 14.8 | −21.1 | 36.9 | 59 |
| VAL | CG2 | A | 210 | 13.5 | −21.4 | 34.7 | 53 |
| VAL | C | A | 210 | 13.9 | −24.3 | 34.9 | 54 |
| VAL | O | A | 210 | 13.9 | −24.2 | 33.7 | 59 |
| GLY | N | A | 211 | 13.1 | −25.1 | 35.5 | 51 |
| GLY | CA | A | 211 | 12.2 | −26.0 | 34.8 | 45 |
| GLY | C | A | 211 | 10.8 | −26.0 | 35.4 | 39 |
| GLY | O | A | 211 | 10.5 | −25.2 | 36.3 | 42 |
| GLY | N | A | 212 | 9.9 | −26.8 | 34.8 | 37 |
| GLY | CA | A | 212 | 8.5 | −26.8 | 35.3 | 33 |
| GLY | C | A | 212 | 7.6 | −27.6 | 34.5 | 32 |
| GLY | O | A | 212 | 8.1 | −28.4 | 33.6 | 31 |
| SER | N | A | 213 | 6.3 | −27.5 | 34.7 | 31 |
| SER | CA | A | 213 | 5.3 | −28.3 | 34.0 | 30 |
| SER | CB | A | 213 | 4.9 | −29.6 | 34.7 | 27 |
| SER | OG | A | 213 | 6.0 | −30.5 | 34.9 | 30 |
| SER | C | A | 213 | 4.0 | −27.6 | 33.6 | 30 |
| SER | O | A | 213 | 3.4 | −27.0 | 34.5 | 28 |
| MET | N | A | 214 | 3.6 | −27.6 | 32.4 | 27 |
| MET | CA | A | 214 | 2.3 | −27.0 | 32.0 | 27 |
| MET | CB | A | 214 | 2.5 | −25.9 | 30.9 | 27 |
| MET | CG | A | 214 | 1.1 | −25.3 | 30.5 | 24 |
| MET | SD | A | 214 | 1.1 | −24.0 | 29.2 | 26 |
| MET | CE | A | 214 | 1.8 | −25.0 | 27.9 | 25 |
| MET | C | A | 214 | 1.4 | −28.1 | 31.6 | 31 |
| MET | O | A | 214 | 1.6 | −28.7 | 30.5 | 32 |
| ILE | N | A | 215 | 0.4 | −28.4 | 32.4 | 29 |
| ILE | CA | A | 215 | −0.5 | −29.4 | 32.1 | 30 |
| ILE | CB | A | 215 | −1.0 | −30.1 | 33.4 | 33 |
| ILE | CG2 | A | 215 | −2.0 | −31.2 | 33.0 | 31 |
| ILE | CG1 | A | 215 | 0.2 | −30.7 | 34.2 | 31 |
| ILE | CD1 | A | 215 | 1.1 | −31.7 | 33.4 | 33 |
| ILE | C | A | 215 | −1.7 | −28.8 | 31.4 | 34 |
| ILE | O | A | 215 | −2.5 | −28.0 | 32.0 | 35 |
| ILE | N | A | 216 | −1.8 | −29.0 | 30.1 | 35 |
| ILE | CA | A | 216 | −2.9 | −28.5 | 29.3 | 37 |
| ILE | CB | A | 216 | −2.5 | −28.3 | 27.8 | 36 |
| ILE | CG2 | A | 216 | −3.7 | −27.8 | 27.0 | 36 |
| ILE | CG1 | A | 216 | −1.4 | −27.3 | 27.6 | 37 |
| ILE | CD1 | A | 216 | −0.1 | −27.7 | 28.3 | 35 |
| ILE | C | A | 216 | −4.2 | −29.4 | 29.3 | 36 |
| ILE | O | A | 216 | −4.1 | −30.5 | 29.0 | 36 |
| GLY | N | A | 217 | −5.3 | −28.8 | 29.8 | 35 |
| GLY | CA | A | 217 | −6.5 | −29.5 | 29.8 | 38 |
| GLY | C | A | 217 | −6.8 | −30.2 | 31.1 | 40 |
| GLY | O | A | 217 | −7.9 | −30.8 | 31.3 | 44 |
| GLY | N | A | 218 | −5.8 | −30.3 | 32.0 | 39 |
| GLY | CA | A | 218 | −6.1 | −31.1 | 33.3 | 39 |
| GLY | C | A | 218 | −5.3 | −30.7 | 34.5 | 39 |
| GLY | O | A | 218 | −4.7 | −29.7 | 34.6 | 39 |
| ILE | N | A | 219 | −5.3 | −31.7 | 35.4 | 39 |
| ILE | CA | A | 219 | −4.6 | −31.5 | 36.7 | 35 |
| ILE | CB | A | 219 | −5.6 | −31.3 | 37.9 | 34 |
| ILE | CG2 | A | 219 | −4.8 | −31.3 | 39.2 | 34 |
| ILE | CG1 | A | 219 | −6.4 | −30.1 | 37.7 | 34 |
| ILE | CD1 | A | 219 | −7.4 | −29.8 | 38.8 | 32 |
| ILE | C | A | 219 | −3.8 | −32.8 | 37.0 | 38 |
| ILE | O | A | 219 | −4.4 | −33.9 | 37.0 | 41 |
| ASP | N | A | 220 | −2.5 | −32.7 | 37.2 | 33 |
| ASP | CA | A | 220 | −1.7 | −33.9 | 37.4 | 35 |
| ASP | CB | A | 220 | −0.3 | −33.9 | 36.7 | 34 |
| ASP | CG | A | 220 | 0.5 | −35.2 | 36.9 | 35 |
| ASP | OD1 | A | 220 | 0.8 | −35.8 | 36.0 | 35 |
| ASP | OD2 | A | 220 | 0.7 | −35.5 | 38.1 | 36 |
| ASP | C | A | 220 | −1.5 | −33.9 | 39.0 | 37 |
| ASP | O | A | 220 | −0.8 | −33.0 | 39.5 | 38 |
| HIS | N | A | 221 | −2.0 | −34.9 | 39.6 | 39 |
| HIS | CA | A | 221 | −2.0 | −35.0 | 41.1 | 38 |
| HIS | CB | A | 221 | −3.1 | −36.0 | 41.6 | 41 |
| HIS | CG | A | 221 | −4.5 | −35.5 | 41.2 | 43 |
| HIS | CD2 | A | 221 | −5.3 | −36.0 | 40.3 | 45 |
| HIS | ND1 | A | 221 | −5.1 | −34.4 | 41.7 | 45 |
| HIS | CE1 | A | 221 | −6.3 | −34.3 | 41.2 | 44 |
| HIS | NE2 | A | 221 | −6.4 | −35.2 | 40.3 | 45 |
| HIS | C | A | 221 | −0.6 | −35.3 | 41.7 | 38 |
| HIS | O | A | 221 | −0.5 | −35.3 | 43.0 | 41 |
| SER | N | A | 222 | 0.4 | −35.5 | 40.9 | 40 |
| SER | CA | A | 222 | 1.7 | −35.7 | 41.5 | 37 |
| SER | CB | A | 222 | 2.5 | −36.8 | 40.6 | 38 |
| SER | OG | A | 222 | 2.7 | −36.3 | 39.3 | 40 |
| SER | C | A | 222 | 2.6 | −34.5 | 41.6 | 37 |
| SER | O | A | 222 | 3.7 | −34.5 | 42.1 | 34 |
| LEU | N | A | 223 | 2.0 | −33.3 | 41.1 | 33 |
| LEU | CA | A | 223 | 2.7 | −32.1 | 41.1 | 29 |
| LEU | CB | A | 223 | 2.4 | −31.2 | 39.8 | 27 |
| LEU | CG | A | 223 | 2.8 | −31.9 | 38.5 | 28 |
| LEU | CD1 | A | 223 | 2.4 | −31.1 | 37.3 | 26 |
| LEU | CD2 | A | 223 | 4.3 | −32.3 | 38.5 | 29 |
| LEU | C | A | 223 | 2.4 | −31.2 | 42.4 | 32 |
| LEU | O | A | 223 | 3.0 | −30.2 | 42.5 | 30 |
| TYR | N | A | 224 | 1.6 | −31.7 | 43.3 | 30 |
| TYR | CA | A | 224 | 1.3 | −31.0 | 44.5 | 32 |
| TYR | CB | A | 224 | 0.1 | −30.1 | 44.4 | 34 |
| TYR | CG | A | 224 | −1.2 | −30.8 | 44.2 | 34 |
| TYR | CD1 | A | 224 | −1.6 | −31.2 | 43.0 | 35 |
| TYR | CE1 | A | 224 | −2.8 | −31.9 | 42.8 | 34 |
| TYR | CD2 | A | 224 | −2.0 | −31.0 | 45.3 | 35 |
| TYR | CE2 | A | 224 | −3.3 | −31.6 | 45.1 | 37 |
| TYR | CZ | A | 224 | −3.7 | −32.0 | 43.9 | 36 |
| TYR | OH | A | 224 | −4.9 | −32.6 | 43.7 | 34 |
| TYR | C | A | 224 | 1.1 | −31.9 | 45.7 | 34 |
| TYR | O | A | 224 | 1.1 | −33.1 | 45.5 | 33 |
| THR | N | A | 225 | 1.0 | −31.3 | 46.9 | 33 |
| THR | CA | A | 225 | 0.8 | −32.1 | 48.1 | 35 |
| THR | CB | A | 225 | 2.1 | −32.2 | 49.0 | 35 |
| THR | OG1 | A | 225 | 2.6 | −30.9 | 49.3 | 34 |
| THR | CG2 | A | 225 | 3.1 | −33.1 | 48.3 | 36 |
| THR | C | A | 225 | −0.3 | −31.4 | 48.9 | 34 |
| THR | O | A | 225 | −0.4 | −30.1 | 48.9 | 35 |
| GLY | N | A | 226 | −1.1 | −32.1 | 49.6 | 34 |
| GLY | CA | A | 226 | −2.2 | −31.6 | 50.4 | 32 |
| GLY | C | A | 226 | −3.4 | −31.2 | 49.6 | 35 |
| GLY | O | A | 226 | −3.7 | −31.9 | 48.5 | 36 |
| SER | N | A | 227 | −4.2 | −30.2 | 50.0 | 33 |
| SER | CA | A | 227 | −5.4 | −29.8 | 49.4 | 35 |
| SER | CB | A | 227 | −6.5 | −29.6 | 50.5 | 35 |
| SER | OG | A | 227 | −6.7 | −30.8 | 51.2 | 40 |
| SER | C | A | 227 | −5.3 | −28.6 | 48.5 | 35 |
| SER | O | A | 227 | −4.5 | −27.7 | 48.7 | 30 |
| LEU | N | A | 228 | −6.1 | −28.6 | 47.4 | 33 |
| LEU | CA | A | 228 | −6.2 | −27.5 | 46.5 | 33 |
| LEU | CB | A | 228 | −6.7 | −27.9 | 45.1 | 32 |
| LEU | CG | A | 228 | −5.7 | −28.9 | 44.3 | 36 |
| LEU | CD1 | A | 228 | −6.3 | −29.4 | 43.0 | 35 |
| LEU | CD2 | A | 228 | −4.4 | −28.1 | 44.0 | 35 |
| LEU | C | A | 228 | −7.3 | −26.5 | 47.0 | 33 |
| LEU | O | A | 228 | −8.4 | −26.9 | 47.3 | 34 |
| TRP | N | A | 229 | −6.9 | −25.2 | 47.1 | 29 |
| TRP | CA | A | 229 | −7.8 | −24.2 | 47.5 | 28 |
| TRP | CB | A | 229 | −7.3 | −23.4 | 48.7 | 28 |
| TRP | CG | A | 229 | −7.3 | −24.2 | 50.0 | 28 |
| TRP | CD2 | A | 229 | −8.3 | −24.2 | 51.0 | 28 |
| TRP | CE2 | A | 229 | −8.0 | −25.1 | 51.9 | 30 |
| TRP | CE3 | A | 229 | −9.5 | −23.4 | 51.2 | 30 |
| TRP | CD1 | A | 229 | −6.4 | −25.1 | 50.3 | 32 |
| TRP | NE1 | A | 229 | −6.8 | −25.7 | 51.5 | 32 |
| TRP | CZ2 | A | 229 | −8.8 | −25.4 | 53.1 | 31 |
| TRP | CZ3 | A | 229 | −10.3 | −23.7 | 52.3 | 30 |
| TRP | CH2 | A | 229 | −9.9 | −24.6 | 53.2 | 28 |
| TRP | C | A | 229 | −8.0 | −23.2 | 46.3 | 31 |
| TRP | O | A | 229 | −7.1 | −22.7 | 45.7 | 29 |
| TYR | N | A | 230 | −9.3 | −22.9 | 46.1 | 29 |
| TYR | CA | A | 230 | −9.7 | −22.1 | 45.0 | 28 |
| TYR | CB | A | 230 | −10.8 | −22.7 | 44.1 | 28 |
| TYR | CG | A | 230 | −10.3 | −24.0 | 43.5 | 26 |
| TYR | CD1 | A | 230 | −10.4 | −25.2 | 44.2 | 26 |
| TYR | CE1 | A | 230 | −10.0 | −26.4 | 43.6 | 27 |
| TYR | CD2 | A | 230 | −9.9 | −24.1 | 42.2 | 27 |
| TYR | CE2 | A | 230 | −9.5 | −25.2 | 41.6 | 25 |
| TYR | CZ | A | 230 | −9.5 | −26.4 | 42.3 | 25 |
| TYR | OH | A | 230 | −9.1 | −27.6 | 41.7 | 27 |
| TYR | C | A | 230 | −10.1 | −20.7 | 45.3 | 26 |
| TYR | O | A | 230 | −10.9 | −20.4 | 46.3 | 29 |
| THR | N | A | 231 | −9.6 | −19.7 | 44.6 | 26 |
| THR | CA | A | 231 | −10.0 | −18.3 | 44.7 | 27 |
| THR | CB | A | 231 | −8.7 | −17.4 | 45.0 | 23 |
| THR | OG1 | A | 231 | −9.1 | −16.1 | 45.3 | 25 |
| THR | CG2 | A | 231 | −7.8 | −17.4 | 43.8 | 22 |
| THR | C | A | 231 | −10.7 | −17.9 | 43.5 | 34 |
| THR | O | A | 231 | −10.2 | −18.1 | 42.4 | 32 |
| PRO | N | A | 232 | −11.9 | −17.2 | 43.6 | 37 |
| PRO | CD | A | 232 | −12.7 | −17.2 | 44.8 | 40 |
| PRO | CA | A | 232 | −12.7 | −16.7 | 42.5 | 38 |
| PRO | CB | A | 232 | −13.8 | −16.0 | 43.2 | 39 |
| PRO | CG | A | 232 | −14.1 | −17.0 | 44.3 | 40 |
| PRO | C | A | 232 | −12.0 | −15.8 | 41.5 | 38 |
| PRO | O | A | 232 | −11.2 | −14.9 | 41.9 | 41 |
| ILE | N | A | 233 | −12.2 | −16.0 | 40.2 | 35 |
| ILE | CA | A | 233 | −11.7 | −15.1 | 39.2 | 32 |
| ILE | CB | A | 233 | −11.5 | −15.7 | 37.8 | 31 |
| ILE | CG2 | A | 233 | −11.2 | −14.7 | 36.8 | 26 |
| ILE | CG1 | A | 233 | −10.5 | −16.9 | 37.9 | 31 |
| ILE | CD1 | A | 233 | −10.3 | −17.6 | 36.5 | 26 |
| ILE | C | A | 233 | −12.7 | −13.9 | 39.2 | 35 |
| ILE | O | A | 233 | −13.8 | −14.1 | 38.8 | 40 |
| ARG | N | A | 234 | −12.2 | −12.8 | 39.7 | 35 |
| ARG | CA | A | 234 | −13.1 | −11.6 | 39.8 | 39 |
| ARG | CB | A | 234 | −12.4 | −10.5 | 40.5 | 33 |
| ARG | CG | A | 234 | −13.2 | −9.2 | 40.7 | 35 |
| ARG | CD | A | 234 | −12.4 | −8.1 | 41.4 | 32 |
| ARG | NE | A | 234 | −13.2 | −6.9 | 41.6 | 34 |
| ARG | CZ | A | 234 | −12.7 | −5.7 | 41.5 | 38 |
| ARG | NH1 | A | 234 | −11.4 | −5.5 | 41.3 | 41 |
| ARG | NH2 | A | 234 | −13.5 | −4.6 | 41.7 | 39 |
| ARG | C | A | 234 | −13.6 | −11.0 | 38.5 | 41 |
| ARG | O | A | 234 | −14.6 | −10.4 | 38.4 | 44 |
| ARG | N | A | 235 | −12.9 | −11.3 | 37.4 | 41 |
| ARG | CA | A | 235 | −13.2 | −10.8 | 36.1 | 39 |
| ARG | CB | A | 235 | −12.9 | −9.3 | 36.1 | 38 |
| ARG | CG | A | 235 | −13.1 | −8.6 | 34.7 | 40 |
| ARG | CD | A | 235 | −12.8 | −7.1 | 34.9 | 39 |
| ARG | NE | A | 235 | −12.9 | −6.3 | 33.6 | 38 |
| ARG | CZ | A | 235 | −12.5 | −5.1 | 33.5 | 40 |
| ARG | NH1 | A | 235 | −11.9 | −4.5 | 34.5 | 43 |
| ARG | NH2 | A | 235 | −12.6 | −4.5 | 32.3 | 42 |
| ARG | C | A | 235 | −12.4 | −11.5 | 35.1 | 40 |
| ARG | O | A | 235 | −11.2 | −11.8 | 35.3 | 43 |
| GLU | N | A | 236 | −13.0 | −11.8 | 33.9 | 41 |
| GLU | CA | A | 236 | −12.2 | −12.5 | 32.9 | 39 |
| GLU | CB | A | 236 | −13.1 | −13.5 | 32.2 | 42 |
| GLU | CG | A | 236 | −13.7 | −14.5 | 33.2 | 44 |
| GLU | CD | A | 236 | −14.6 | −15.5 | 32.6 | 46 |
| GLU | OE1 | A | 236 | −15.8 | −15.4 | 32.7 | 50 |
| GLU | OE2 | A | 236 | −14.1 | −16.4 | 31.9 | 49 |
| GLU | C | A | 236 | −11.6 | −11.5 | 31.9 | 40 |
| GLU | O | A | 236 | −12.2 | −11.1 | 30.9 | 43 |
| TRP | N | A | 237 | −10.3 | −11.2 | 32.2 | 36 |
| TRP | CA | A | 237 | −9.5 | −10.3 | 31.4 | 34 |
| TRP | CB | A | 237 | −9.8 | −8.8 | 31.6 | 29 |
| TRP | CG | A | 237 | −9.6 | −8.2 | 33.0 | 28 |
| TRP | CD2 | A | 237 | −9.2 | −6.9 | 33.3 | 28 |
| TRP | CE2 | A | 237 | −9.2 | −6.8 | 34.7 | 27 |
| TRP | CE3 | A | 237 | −8.9 | −5.8 | 32.6 | 31 |
| TRP | CD1 | A | 237 | −9.8 | −8.9 | 34.2 | 28 |
| TRP | NE1 | A | 237 | −9.6 | −8.0 | 35.2 | 25 |
| TRP | CZ2 | A | 237 | −8.9 | −5.6 | 35.4 | 26 |
| TRP | CZ3 | A | 237 | −8.5 | −4.6 | 33.2 | 32 |
| TRP | CH2 | A | 237 | −8.6 | −4.5 | 34.6 | 29 |
| TRP | C | A | 237 | −8.1 | −10.8 | 31.8 | 31 |
| TRP | O | A | 237 | −7.7 | −11.8 | 31.3 | 32 |
| TYR | N | A | 238 | −7.5 | −10.2 | 32.8 | 29 |
| TYR | CA | A | 238 | −6.2 | −10.8 | 33.3 | 28 |
| TYR | CB | A | 238 | −5.5 | −9.8 | 34.2 | 24 |
| TYR | CG | A | 238 | −4.9 | −8.6 | 33.5 | 25 |
| TYR | CD1 | A | 238 | −3.7 | −8.6 | 32.9 | 26 |
| TYR | CE1 | A | 238 | −3.2 | −7.5 | 32.3 | 26 |
| TYR | CD2 | A | 238 | −5.7 | −7.4 | 33.5 | 27 |
| TYR | CE2 | A | 238 | −5.1 | −6.2 | 32.9 | 27 |
| TYR | CZ | A | 238 | −3.9 | −6.3 | 32.3 | 25 |
| TYR | OH | A | 238 | −3.4 | −5.2 | 31.7 | 26 |
| TYR | C | A | 238 | −6.8 | −11.8 | 34.2 | 26 |
| TYR | O | A | 238 | −8.0 | −11.8 | 34.4 | 30 |
| TYR | N | A | 239 | −6.0 | −12.7 | 34.8 | 31 |
| TYR | CA | A | 239 | −6.6 | −13.6 | 35.8 | 29 |
| TYR | CB | A | 239 | −5.8 | −14.9 | 35.9 | 27 |
| TYR | CG | A | 239 | −5.9 | −15.8 | 34.7 | 28 |
| TYR | CD1 | A | 239 | −5.0 | −15.7 | 33.6 | 26 |
| TYR | CE1 | A | 239 | −5.1 | −16.5 | 32.5 | 25 |
| TYR | CD2 | A | 239 | −6.9 | −16.8 | 34.6 | 27 |
| TYR | CE2 | A | 239 | −7.1 | −17.6 | 33.4 | 26 |
| TYR | CZ | A | 239 | −6.2 | −17.4 | 32.4 | 27 |
| TYR | OH | A | 239 | −6.3 | −18.2 | 31.3 | 20 |
| TYR | C | A | 239 | −6.5 | −12.8 | 37.1 | 31 |
| TYR | O | A | 239 | −5.5 | −12.8 | 37.8 | 31 |
| GLU | N | A | 240 | −7.6 | −12.2 | 37.4 | 31 |
| GLU | CA | A | 240 | −7.8 | −11.3 | 38.5 | 33 |
| GLU | CB | A | 240 | −8.6 | −10.1 | 38.3 | 33 |
| GLU | CG | A | 240 | −8.7 | −9.2 | 39.5 | 33 |
| GLU | CD | A | 240 | −9.6 | −8.0 | 39.2 | 35 |
| GLU | OE1 | A | 240 | −10.2 | −7.9 | 38.1 | 38 |
| GLU | OE2 | A | 240 | −9.7 | −7.1 | 40.0 | 40 |
| GLU | C | A | 240 | −8.3 | −12.1 | 39.8 | 30 |
| GLU | O | A | 240 | −9.3 | −12.8 | 39.7 | 27 |
| VAL | N | A | 241 | −7.6 | −11.9 | 40.9 | 29 |
| VAL | CA | A | 241 | −8.0 | −12.5 | 42.1 | 29 |
| VAL | CB | A | 241 | −7.0 | −13.5 | 42.6 | 28 |
| VAL | CG1 | A | 241 | −6.9 | −14.7 | 41.6 | 26 |
| VAL | CG2 | A | 241 | −5.6 | −12.9 | 42.9 | 27 |
| VAL | C | A | 241 | −8.1 | −11.3 | 43.2 | 30 |
| VAL | O | A | 241 | −7.8 | −10.2 | 42.9 | 28 |
| ILE | N | A | 242 | −8.6 | −11.7 | 44.4 | 26 |
| ILE | CA | A | 242 | −8.8 | −10.7 | 45.4 | 31 |
| ILE | CB | A | 242 | −10.2 | −10.5 | 45.7 | 31 |
| ILE | CG2 | A | 242 | −10.4 | −9.4 | 46.9 | 32 |
| ILE | CG1 | A | 242 | −11.0 | −10.0 | 44.5 | 33 |
| ILE | CD1 | A | 242 | −12.5 | −9.7 | 44.7 | 35 |
| ILE | C | A | 242 | −8.0 | −11.1 | 46.7 | 30 |
| ILE | O | A | 242 | −8.2 | −12.1 | 47.3 | 35 |
| ILE | N | A | 243 | −7.1 | −10.2 | 47.1 | 29 |
| ILE | CA | A | 243 | −6.3 | −10.4 | 48.3 | 30 |
| ILE | CB | A | 243 | −4.9 | −9.9 | 48.2 | 28 |
| ILE | CG2 | A | 243 | −4.1 | −10.0 | 49.5 | 26 |
| ILE | CG1 | A | 243 | −4.2 | −10.6 | 47.0 | 26 |
| ILE | CD1 | A | 243 | −2.8 | −10.1 | 46.7 | 29 |
| ILE | C | A | 243 | −7.0 | −9.7 | 49.5 | 31 |
| ILE | O | A | 243 | −7.4 | −8.6 | 49.4 | 31 |
| VAL | N | A | 244 | −7.2 | −10.4 | 50.6 | 30 |
| VAL | CA | A | 244 | −8.0 | −9.8 | 51.7 | 32 |
| VAL | CB | A | 244 | −9.2 | −10.7 | 52.0 | 31 |
| VAL | CG1 | A | 244 | −10.1 | −10.7 | 50.8 | 31 |
| VAL | CG2 | A | 244 | −8.8 | −12.1 | 52.6 | 26 |
| VAL | C | A | 244 | −7.2 | −9.6 | 53.0 | 30 |
| VAL | O | A | 244 | −7.7 | −9.0 | 54.0 | 33 |
| ARG | N | A | 245 | −5.9 | −10.0 | 53.0 | 30 |
| ARG | CA | A | 245 | −5.0 | −9.9 | 54.2 | 27 |
| ARG | CB | A | 245 | −5.6 | −10.7 | 55.3 | 25 |
| ARG | CG | A | 245 | −4.7 | −10.8 | 56.6 | 31 |
| ARG | CD | A | 245 | −5.3 | −11.7 | 57.7 | 31 |
| ARG | NE | A | 245 | −4.4 | −11.9 | 58.8 | 31 |
| ARG | CZ | A | 245 | −4.5 | −11.2 | 60.0 | 32 |
| ARG | NH1 | A | 245 | −5.5 | −10.4 | 60.2 | 30 |
| ARG | NH2 | A | 245 | −3.7 | −11.5 | 61.0 | 26 |
| ARG | C | A | 245 | −3.6 | −10.3 | 53.8 | 28 |
| ARG | O | A | 245 | −3.4 | −11.3 | 53.1 | 24 |
| VAL | N | A | 246 | −2.6 | −9.6 | 54.4 | 27 |
| VAL | CA | A | 246 | −1.2 | −9.8 | 54.2 | 22 |
| VAL | CB | A | 246 | −0.5 | −8.8 | 53.2 | 19 |
| VAL | CG1 | A | 246 | 1.0 | −9.2 | 53.0 | 13 |
| VAL | CG2 | A | 246 | −1.3 | −8.7 | 51.9 | 17 |
| VAL | C | A | 246 | −0.5 | −9.9 | 55.5 | 25 |
| VAL | O | A | 246 | −0.6 | −9.1 | 56.4 | 25 |
| GLU | N | A | 247 | 0.4 | −11.0 | 55.7 | 21 |
| GLU | CA | A | 247 | 1.1 | −11.2 | 56.9 | 19 |
| GLU | CB | A | 247 | 0.6 | −12.4 | 57.7 | 17 |
| GLU | CG | A | 247 | −0.9 | −12.2 | 58.2 | 21 |
| GLU | CD | A | 247 | −1.3 | −13.5 | 58.9 | 25 |
| GLU | OE1 | A | 247 | −0.5 | −14.4 | 59.0 | 22 |
| GLU | OE2 | A | 247 | −2.4 | −13.5 | 59.4 | 22 |
| GLU | C | A | 247 | 2.6 | −11.4 | 56.6 | 22 |
| GLU | O | A | 247 | 3.0 | −12.0 | 55.6 | 18 |
| ILE | N | A | 248 | 3.5 | −10.9 | 57.5 | 21 |
| ILE | CA | A | 248 | 4.9 | −11.1 | 57.3 | 23 |
| ILE | CB | A | 248 | 5.7 | −9.8 | 57.2 | 19 |
| ILE | CG2 | A | 248 | 7.2 | −10.1 | 57.1 | 20 |
| ILE | CG1 | A | 248 | 5.2 | −8.9 | 56.1 | 22 |
| ILE | CD1 | A | 248 | 5.3 | −9.6 | 54.7 | 22 |
| ILE | C | A | 248 | 5.2 | −11.8 | 58.7 | 22 |
| ILE | O | A | 248 | 5.0 | −11.2 | 59.7 | 20 |
| ASN | N | A | 249 | 5.5 | −13.1 | 58.7 | 21 |
| ASN | CA | A | 249 | 5.7 | −13.8 | 60.0 | 21 |
| ASN | CB | A | 249 | 6.9 | −13.6 | 60.7 | 18 |
| ASN | CG | A | 249 | 8.1 | −14.3 | 60.1 | 22 |
| ASN | OD1 | A | 249 | 8.0 | −15.1 | 59.2 | 23 |
| ASN | ND2 | A | 249 | 9.3 | −14.0 | 60.7 | 24 |
| ASN | C | A | 249 | 4.5 | −13.8 | 60.9 | 23 |
| ASN | O | A | 249 | 4.6 | −13.7 | 62.1 | 24 |
| GLY | N | A | 250 | 3.3 | −13.8 | 60.3 | 22 |
| GLY | CA | A | 250 | 2.1 | −13.8 | 61.1 | 21 |
| GLY | C | A | 250 | 1.7 | −12.4 | 61.5 | 24 |
| GLY | O | A | 250 | 0.6 | −12.2 | 62.0 | 27 |
| GLN | N | A | 251 | 2.5 | −11.4 | 61.3 | 20 |
| GLN | CA | A | 251 | 2.2 | −10.1 | 61.6 | 25 |
| GLN | CB | A | 251 | 3.3 | −9.2 | 62.1 | 23 |
| GLN | CG | A | 251 | 2.9 | −7.8 | 62.5 | 31 |
| GLN | CD | A | 251 | 4.0 | −7.0 | 63.0 | 31 |
| GLN | OE1 | A | 251 | 5.2 | −7.3 | 62.8 | 40 |
| GLN | NE2 | A | 251 | 3.7 | −5.8 | 63.6 | 36 |
| GLN | C | A | 251 | 1.4 | −9.4 | 60.5 | 21 |
| GLN | O | A | 251 | 1.9 | −9.3 | 59.4 | 23 |
| ASP | N | A | 252 | 0.2 | −9.0 | 60.8 | 24 |
| ASP | CA | A | 252 | −0.7 | −8.3 | 59.9 | 27 |
| ASP | CB | A | 252 | −2.1 | −8.2 | 60.6 | 29 |
| ASP | CG | A | 252 | −3.1 | −7.5 | 59.7 | 30 |
| ASP | OD1 | A | 252 | −2.9 | −7.4 | 58.5 | 30 |
| ASP | OD2 | A | 252 | −4.2 | −7.1 | 60.2 | 33 |
| ASP | C | A | 252 | −0.2 | −7.0 | 59.4 | 28 |
| ASP | O | A | 252 | 0.1 | −6.1 | 60.2 | 29 |
| LEU | N | A | 253 | −0.2 | −6.8 | 58.1 | 28 |
| LEU | CA | A | 253 | 0.2 | −5.4 | 57.6 | 32 |
| LEU | CB | A | 253 | 0.4 | −5.4 | 56.0 | 32 |
| LEU | CG | A | 253 | 1.6 | −6.2 | 55.5 | 36 |
| LEU | CD1 | A | 253 | 1.7 | −6.1 | 54.0 | 36 |
| LEU | CD2 | A | 253 | 2.9 | −5.8 | 56.2 | 35 |
| LEU | C | A | 253 | −0.8 | −4.4 | 58.0 | 31 |
| LEU | O | A | 253 | −0.6 | −3.2 | 58.0 | 35 |
| LYS | N | A | 254 | −2.0 | −4.9 | 58.4 | 32 |
| LYS | CA | A | 254 | −3.1 | −4.0 | 58.9 | 42 |
| LYS | CB | A | 254 | −2.7 | −3.5 | 60.3 | 46 |
| LYS | CG | A | 254 | −3.7 | −2.6 | 61.0 | 55 |
| LYS | CD | A | 254 | −3.1 | −2.2 | 62.4 | 58 |
| LYS | CE | A | 254 | −4.1 | −1.3 | 63.1 | 62 |
| LYS | NZ | A | 254 | −5.4 | −1.9 | 63.4 | 64 |
| LYS | C | A | 254 | −3.6 | −2.9 | 58.0 | 42 |
| LYS | O | A | 254 | −4.2 | −1.9 | 58.4 | 42 |
| MET | N | A | 255 | −3.4 | −3.1 | 56.7 | 39 |
| MET | CA | A | 255 | −3.8 | −2.1 | 55.7 | 38 |
| MET | CB | A | 255 | −2.9 | −2.0 | 54.5 | 36 |
| MET | CG | A | 255 | −1.5 | −1.7 | 54.7 | 38 |
| MET | SD | A | 255 | −0.6 | −1.7 | 53.2 | 36 |
| MET | CE | A | 255 | −1.0 | −0.1 | 52.5 | 38 |
| MET | C | A | 255 | −5.3 | −2.3 | 55.2 | 37 |
| MET | O | A | 255 | −5.8 | −3.4 | 55.3 | 41 |
| ASP | N | A | 256 | −5.9 | −1.2 | 54.8 | 35 |
| ASP | CA | A | 256 | −7.3 | −1.4 | 54.2 | 37 |
| ASP | CB | A | 256 | −7.8 | −0.1 | 53.7 | 42 |
| ASP | CG | A | 256 | −9.2 | −0.2 | 53.0 | 50 |
| ASP | OD1 | A | 256 | −9.6 | −1.3 | 52.9 | 51 |
| ASP | OD2 | A | 256 | −9.8 | 0.8 | 52.7 | 57 |
| ASP | C | A | 256 | −7.0 | −2.4 | 53.1 | 36 |
| ASP | O | A | 256 | −6.2 | −2.2 | 52.2 | 32 |
| CYS | N | A | 257 | −7.8 | −3.5 | 53.0 | 38 |
| CYS | CA | A | 257 | −7.6 | −4.5 | 52.0 | 42 |
| CYS | C | A | 257 | −7.7 | −4.0 | 50.5 | 43 |
| CYS | O | A | 257 | −7.3 | −4.8 | 49.6 | 44 |
| CYS | CB | A | 257 | −8.4 | −5.7 | 52.2 | 44 |
| CYS | SG | A | 257 | −10.2 | −5.4 | 52.1 | 50 |
| LYS | N | A | 258 | −8.2 | −2.8 | 50.3 | 43 |
| LYS | CA | A | 258 | −8.3 | −2.4 | 48.9 | 43 |
| LYS | CB | A | 258 | −9.2 | −1.2 | 48.6 | 47 |
| LYS | CG | A | 258 | −10.7 | −1.5 | 48.8 | 52 |
| LYS | CD | A | 258 | −11.5 | −0.2 | 48.4 | 55 |
| LYS | CE | A | 258 | −13.0 | −0.3 | 48.6 | 56 |
| LYS | NZ | A | 258 | −13.4 | −0.6 | 50.0 | 62 |
| LYS | C | A | 258 | −6.9 | −2.0 | 48.4 | 41 |
| LYS | O | A | 258 | −6.6 | −1.9 | 47.2 | 40 |
| GLU | N | A | 259 | −6.0 | −1.7 | 49.4 | 36 |
| GLU | CA | A | 259 | −4.6 | −1.3 | 49.0 | 37 |
| GLU | CB | A | 259 | −3.9 | −0.7 | 50.2 | 40 |
| GLU | CG | A | 259 | −4.5 | 0.5 | 50.9 | 45 |
| GLU | CD | A | 259 | −4.6 | 1.7 | 49.9 | 46 |
| GLU | OE1 | A | 259 | −3.9 | 1.6 | 48.9 | 50 |
| GLU | OE2 | A | 259 | −5.2 | 2.7 | 50.2 | 46 |
| GLU | C | A | 259 | −3.9 | −2.5 | 48.4 | 35 |
| GLU | O | A | 259 | −3.0 | −2.3 | 47.6 | 32 |
| TYR | N | A | 260 | −4.3 | −3.7 | 48.8 | 33 |
| TYR | CA | A | 260 | −3.6 | −4.9 | 48.3 | 32 |
| TYR | CB | A | 260 | −4.0 | −6.1 | 49.2 | 29 |
| TYR | CG | A | 260 | −3.6 | −5.9 | 50.7 | 24 |
| TYR | CD1 | A | 260 | −4.4 | −6.5 | 51.7 | 24 |
| TYR | CE1 | A | 260 | −4.1 | −6.3 | 53.0 | 28 |
| TYR | CD2 | A | 260 | −2.5 | −5.2 | 51.1 | 26 |
| TYR | CE2 | A | 260 | −2.1 | −5.1 | 52.4 | 22 |
| TYR | CZ | A | 260 | −2.9 | −5.6 | 53.4 | 26 |
| TYR | OH | A | 260 | −2.6 | −5.5 | 54.7 | 32 |
| TYR | C | A | 260 | −4.1 | −5.2 | 46.9 | 35 |
| TYR | O | A | 260 | −3.5 | −6.1 | 46.3 | 34 |
| ASN | N | A | 261 | −5.1 | −4.6 | 46.5 | 35 |
| ASN | CA | A | 261 | −5.7 | −4.8 | 45.1 | 36 |
| ASN | CB | A | 261 | −7.0 | −5.6 | 45.2 | 38 |
| ASN | CG | A | 261 | −6.9 | −6.8 | 46.0 | 37 |
| ASN | OD1 | A | 261 | −6.8 | −7.9 | 45.5 | 37 |
| ASN | ND2 | A | 261 | −7.1 | −6.7 | 47.3 | 41 |
| ASN | C | A | 261 | −5.8 | −3.6 | 44.3 | 33 |
| ASN | O | A | 261 | −6.5 | −3.5 | 43.3 | 39 |
| TYR | N | A | 262 | −5.1 | −2.5 | 44.7 | 34 |
| TYR | CA | A | 262 | −5.1 | −1.3 | 43.9 | 37 |
| TYR | CB | A | 262 | −4.9 | −0.1 | 44.9 | 37 |
| TYR | CG | A | 262 | −4.8 | 1.2 | 44.2 | 42 |
| TYR | CD1 | A | 262 | −5.9 | 1.8 | 43.5 | 43 |
| TYR | CE1 | A | 262 | −5.8 | 3.0 | 42.8 | 48 |
| TYR | CD2 | A | 262 | −3.6 | 2.0 | 44.2 | 47 |
| TYR | CE2 | A | 262 | −3.5 | 3.2 | 43.5 | 49 |
| TYR | CZ | A | 262 | −4.6 | 3.7 | 42.9 | 51 |
| TYR | OH | A | 262 | −4.5 | 4.9 | 42.2 | 55 |
| TYR | C | A | 262 | −4.0 | −1.3 | 42.8 | 38 |
| TYR | O | A | 262 | −2.8 | −1.2 | 43.1 | 39 |
| ASP | N | A | 263 | −4.4 | −1.3 | 41.6 | 38 |
| ASP | CA | A | 263 | −5.8 | −1.2 | 41.2 | 40 |
| ASP | CB | A | 263 | −6.0 | −0.2 | 40.1 | 45 |
| ASP | CG | A | 263 | −5.2 | −0.4 | 38.8 | 47 |
| ASP | OD1 | A | 263 | −4.4 | −1.4 | 38.8 | 48 |
| ASP | OD2 | A | 263 | −5.3 | 0.3 | 37.9 | 49 |
| ASP | C | A | 263 | −6.4 | −2.6 | 40.7 | 37 |
| ASP | O | A | 263 | −7.6 | −2.7 | 40.3 | 38 |
| LYS | N | A | 264 | −5.6 | −3.6 | 40.9 | 34 |
| LYS | CA | A | 264 | −6.0 | −5.0 | 40.6 | 32 |
| LYS | CB | A | 264 | −6.3 | −5.2 | 39.1 | 31 |
| LYS | CG | A | 264 | −5.0 | −5.0 | 38.2 | 33 |
| LYS | CD | A | 264 | −5.3 | −5.1 | 36.7 | 34 |
| LYS | CE | A | 264 | −4.0 | −4.9 | 36.0 | 35 |
| LYS | NZ | A | 264 | −3.5 | −3.5 | 36.2 | 37 |
| LYS | C | A | 264 | −4.9 | −6.0 | 41.0 | 32 |
| LYS | O | A | 264 | −3.8 | −5.6 | 41.2 | 32 |
| SER | N | A | 265 | −5.3 | −7.2 | 41.2 | 27 |
| SER | CA | A | 265 | −4.3 | −8.2 | 41.6 | 28 |
| SER | CB | A | 265 | −4.5 | −8.8 | 43.0 | 25 |
| SER | OG | A | 265 | −4.4 | −7.8 | 44.0 | 25 |
| SER | C | A | 265 | −4.5 | −9.3 | 40.6 | 25 |
| SER | O | A | 265 | −5.6 | −9.8 | 40.3 | 25 |
| ILE | N | A | 266 | −3.3 | −9.7 | 40.0 | 25 |
| ILE | CA | A | 266 | −3.3 | −10.8 | 39.0 | 27 |
| ILE | CB | A | 266 | −3.2 | −10.2 | 37.5 | 26 |
| ILE | CG2 | A | 266 | −4.2 | −9.1 | 37.3 | 24 |
| ILE | CG1 | A | 266 | −1.8 | −9.6 | 37.4 | 23 |
| ILE | CD1 | A | 266 | −1.5 | −9.0 | 36.0 | 25 |
| ILE | C | A | 266 | −2.3 | −11.8 | 39.1 | 27 |
| ILE | O | A | 266 | −1.2 | −11.6 | 39.8 | 27 |
| VAL | N | A | 267 | −2.5 | −13.0 | 38.6 | 25 |
| VAL | CA | A | 267 | −1.5 | −14.1 | 38.6 | 26 |
| VAL | CB | A | 267 | −2.1 | −15.5 | 38.9 | 25 |
| VAL | CG1 | A | 267 | −1.1 | −16.6 | 38.9 | 28 |
| VAL | CG2 | A | 267 | −3.0 | −15.5 | 40.2 | 24 |
| VAL | C | A | 267 | −0.9 | −14.0 | 37.3 | 28 |
| VAL | O | A | 267 | −1.5 | −14.2 | 36.2 | 28 |
| ASP | N | A | 268 | 0.4 | −13.8 | 37.2 | 27 |
| ASP | CA | A | 268 | 1.1 | −13.7 | 36.0 | 25 |
| ASP | CB | A | 268 | 1.4 | −12.2 | 35.6 | 22 |
| ASP | CG | A | 268 | 2.1 | −12.0 | 34.3 | 29 |
| ASP | OD1 | A | 268 | 2.2 | −13.0 | 33.5 | 25 |
| ASP | OD2 | A | 268 | 2.6 | −10.9 | 34.1 | 26 |
| ASP | C | A | 268 | 2.4 | −14.5 | 35.9 | 24 |
| ASP | O | A | 268 | 3.4 | −14.1 | 36.5 | 24 |
| SER | N | A | 269 | 2.5 | −15.6 | 35.1 | 21 |
| SER | CA | A | 269 | 3.6 | −16.4 | 34.9 | 18 |
| SER | CB | A | 269 | 3.3 | −17.7 | 34.3 | 23 |
| SER | OG | A | 269 | 2.8 | −17.5 | 33.0 | 28 |
| SER | C | A | 269 | 4.7 | −15.7 | 34.1 | 22 |
| SER | O | A | 269 | 5.9 | −16.1 | 34.1 | 22 |
| GLY | N | A | 270 | 4.4 | −14.5 | 33.5 | 21 |
| GLY | CA | A | 270 | 5.3 | −13.8 | 32.7 | 19 |
| GLY | C | A | 270 | 6.0 | −12.7 | 33.5 | 26 |
| GLY | O | A | 270 | 6.7 | −11.8 | 33.0 | 23 |
| THR | N | A | 271 | 5.8 | −12.7 | 34.8 | 24 |
| THR | CA | A | 271 | 6.5 | −11.8 | 35.8 | 24 |
| THR | CB | A | 271 | 5.5 | −10.9 | 36.5 | 28 |
| THR | OG1 | A | 271 | 4.7 | −10.1 | 35.6 | 28 |
| THR | CG2 | A | 271 | 6.2 | −10.1 | 37.6 | 30 |
| THR | C | A | 271 | 7.4 | −12.5 | 36.7 | 25 |
| THR | O | A | 271 | 7.0 | −13.5 | 37.4 | 23 |
| THR | N | A | 272 | 8.6 | −12.0 | 36.8 | 24 |
| THR | CA | A | 272 | 9.6 | −12.6 | 37.7 | 25 |
| THR | CB | A | 272 | 11.0 | −12.1 | 37.5 | 25 |
| THR | OG1 | A | 272 | 11.4 | −12.3 | 36.1 | 26 |
| THR | CG2 | A | 272 | 12.1 | −12.7 | 38.4 | 22 |
| THR | C | A | 272 | 9.3 | −12.4 | 39.2 | 25 |
| THR | O | A | 272 | 9.1 | −13.4 | 39.9 | 29 |
| ASN | N | A | 273 | 9.1 | −11.2 | 39.5 | 22 |
| ASN | CA | A | 273 | 8.8 | −10.8 | 40.9 | 25 |
| ASN | CB | A | 273 | 9.4 | −9.4 | 41.2 | 26 |
| ASN | CG | A | 273 | 10.9 | −9.3 | 41.0 | 29 |
| ASN | OD1 | A | 273 | 11.5 | −10.2 | 40.5 | 21 |
| ASN | ND2 | A | 273 | 11.5 | −8.1 | 41.4 | 31 |
| ASN | C | A | 273 | 7.4 | −10.8 | 41.4 | 27 |
| ASN | O | A | 273 | 6.5 | −11.1 | 40.7 | 27 |
| LEU | N | A | 274 | 7.3 | −10.5 | 42.7 | 24 |
| LEU | CA | A | 274 | 6.0 | −10.3 | 43.4 | 22 |
| LEU | CB | A | 274 | 6.1 | −10.8 | 44.9 | 23 |
| LEU | CG | A | 274 | 4.8 | −10.5 | 45.6 | 24 |
| LEU | CD1 | A | 274 | 3.6 | −11.1 | 45.0 | 26 |
| LEU | CD2 | A | 274 | 4.9 | −11.0 | 47.1 | 25 |
| LEU | C | A | 274 | 6.0 | −8.8 | 43.3 | 23 |
| LEU | O | A | 274 | 6.7 | −8.1 | 44.0 | 24 |
| ARG | N | A | 275 | 5.0 | −8.3 | 42.5 | 25 |
| ARG | CA | A | 275 | 4.9 | −6.9 | 42.4 | 24 |
| ARG | CB | A | 275 | 4.5 | −6.5 | 40.9 | 29 |
| ARG | CG | A | 275 | 5.5 | −7.0 | 39.9 | 34 |
| ARG | CD | A | 275 | 6.9 | −6.4 | 39.9 | 36 |
| ARG | NE | A | 275 | 7.0 | −5.0 | 39.8 | 33 |
| ARG | CZ | A | 275 | 7.0 | −4.3 | 38.7 | 32 |
| ARG | NH1 | A | 275 | 7.1 | −3.0 | 38.6 | 31 |
| ARG | NH2 | A | 275 | 6.9 | −5.0 | 37.5 | 38 |
| ARG | C | A | 275 | 3.8 | −6.4 | 43.4 | 23 |
| ARG | O | A | 275 | 2.7 | −6.9 | 43.4 | 22 |
| LEU | N | A | 276 | 4.1 | −5.4 | 44.2 | 22 |
| LEU | CA | A | 276 | 3.2 | −4.8 | 45.2 | 23 |
| LEU | CB | A | 276 | 3.6 | −5.1 | 46.6 | 23 |
| LEU | CG | A | 276 | 3.7 | −6.5 | 47.0 | 24 |
| LEU | CD1 | A | 276 | 4.3 | −6.7 | 48.4 | 23 |
| LEU | CD2 | A | 276 | 2.4 | −7.2 | 46.9 | 25 |
| LEU | C | A | 276 | 2.9 | −3.3 | 45.0 | 25 |
| LEU | O | A | 276 | 3.8 | −2.5 | 44.7 | 23 |
| PRO | N | A | 277 | 1.6 | −2.9 | 45.1 | 29 |
| PRO | CD | A | 277 | 0.4 | −3.8 | 45.2 | 28 |
| PRO | CA | A | 277 | 1.2 | −1.5 | 45.0 | 29 |
| PRO | CB | A | 277 | −0.2 | −1.6 | 45.5 | 27 |
| PRO | CG | A | 277 | −0.7 | −2.8 | 44.8 | 29 |
| PRO | C | A | 277 | 2.1 | −0.7 | 45.9 | 31 |
| PRO | O | A | 277 | 2.4 | −1.1 | 47.0 | 28 |
| LYS | N | A | 278 | 2.6 | 0.4 | 45.4 | 34 |
| LYS | CA | A | 278 | 3.5 | 1.3 | 46.1 | 38 |
| LYS | CB | A | 278 | 3.4 | 2.8 | 45.6 | 43 |
| LYS | CG | A | 278 | 4.3 | 3.7 | 46.3 | 44 |
| LYS | CD | A | 278 | 4.3 | 5.1 | 45.8 | 48 |
| LYS | CE | A | 278 | 5.2 | 6.1 | 46.6 | 50 |
| LYS | NZ | A | 278 | 5.1 | 7.5 | 46.1 | 53 |
| LYS | C | A | 278 | 3.3 | 1.4 | 47.6 | 39 |
| LYS | O | A | 278 | 4.3 | 1.3 | 48.4 | 37 |
| LYS | N | A | 279 | 2.1 | 1.5 | 48.1 | 36 |
| LYS | CA | A | 279 | 1.9 | 1.6 | 49.6 | 39 |
| LYS | CB | A | 279 | 0.5 | 2.2 | 49.9 | 42 |
| LYS | CG | A | 279 | 0.3 | 3.6 | 49.4 | 52 |
| LYS | CD | A | 279 | −1.1 | 4.2 | 49.7 | 54 |
| LYS | CE | A | 279 | −1.3 | 4.2 | 51.2 | 55 |
| LYS | NZ | A | 279 | −2.6 | 4.7 | 51.6 | 56 |
| LYS | C | A | 279 | 2.0 | 0.2 | 50.3 | 39 |
| LYS | O | A | 279 | 2.4 | 0.2 | 51.5 | 38 |
| VAL | N | A | 280 | 1.8 | −0.8 | 49.5 | 35 |
| VAL | CA | A | 280 | 1.9 | −2.2 | 50.1 | 30 |
| VAL | CB | A | 280 | 1.0 | −3.2 | 49.4 | 25 |
| VAL | CG1 | A | 280 | 1.2 | −4.5 | 50.0 | 22 |
| VAL | CG2 | A | 280 | −0.4 | −2.7 | 49.4 | 22 |
| VAL | C | A | 280 | 3.4 | −2.6 | 50.1 | 31 |
| VAL | O | A | 280 | 3.9 | −3.2 | 51.0 | 34 |
| PHE | N | A | 281 | 4.1 | −2.1 | 49.1 | 27 |
| PHE | CA | A | 281 | 5.5 | −2.4 | 48.9 | 31 |
| PHE | CB | A | 281 | 6.1 | −1.9 | 47.6 | 27 |
| PHE | CG | A | 281 | 7.6 | −2.2 | 47.5 | 31 |
| PHE | CD1 | A | 281 | 8.0 | −3.5 | 47.2 | 29 |
| PHE | CD2 | A | 281 | 8.5 | −1.2 | 47.6 | 30 |
| PHE | CE1 | A | 281 | 9.4 | −3.7 | 47.1 | 30 |
| PHE | CE2 | A | 281 | 9.8 | −1.4 | 47.6 | 30 |
| PHE | CZ | A | 281 | 10.3 | −2.7 | 47.3 | 29 |
| PHE | C | A | 281 | 6.3 | −1.8 | 50.1 | 29 |
| PHE | O | A | 281 | 7.1 | −2.4 | 50.8 | 29 |
| GLU | N | A | 282 | 6.1 | −0.5 | 50.3 | 32 |
| GLU | CA | A | 282 | 6.8 | 0.2 | 51.3 | 35 |
| GLU | CB | A | 282 | 6.4 | 1.7 | 51.3 | 41 |
| GLU | CG | A | 282 | 6.8 | 2.5 | 50.0 | 47 |
| GLU | CD | A | 282 | 8.3 | 2.4 | 49.7 | 55 |
| GLU | OE1 | A | 282 | 9.0 | 2.0 | 50.5 | 56 |
| GLU | OE2 | A | 282 | 8.6 | 2.8 | 48.5 | 58 |
| GLU | C | A | 282 | 6.5 | −0.4 | 52.7 | 33 |
| GLU | O | A | 282 | 7.4 | −0.4 | 53.5 | 32 |
| ALA | N | A | 283 | 5.3 | −0.8 | 53.0 | 32 |
| ALA | CA | A | 283 | 5.0 | −1.4 | 54.3 | 29 |
| ALA | CB | A | 283 | 3.5 | −1.4 | 54.5 | 30 |
| ALA | C | A | 283 | 5.5 | −2.9 | 54.4 | 32 |
| ALA | O | A | 283 | 5.9 | −3.3 | 55.5 | 34 |
| ALA | N | A | 284 | 5.5 | −3.6 | 53.3 | 23 |
| ALA | CA | A | 284 | 5.9 | −5.0 | 53.3 | 25 |
| ALA | CB | A | 284 | 5.6 | −5.7 | 52.0 | 17 |
| ALA | C | A | 284 | 7.4 | −5.0 | 53.5 | 27 |
| ALA | O | A | 284 | 8.0 | −5.8 | 54.2 | 30 |
| VAL | N | A | 285 | 8.1 | −4.1 | 52.8 | 27 |
| VAL | CA | A | 285 | 9.6 | −4.0 | 52.9 | 30 |
| VAL | CB | A | 285 | 10.2 | −3.1 | 51.8 | 34 |
| VAL | CG1 | A | 285 | 11.7 | −3.0 | 51.9 | 38 |
| VAL | CG2 | A | 285 | 9.8 | −3.6 | 50.4 | 33 |
| VAL | C | A | 285 | 10.0 | −3.5 | 54.2 | 30 |
| VAL | O | A | 285 | 11.1 | −3.8 | 54.7 | 30 |
| LYS | N | A | 286 | 9.2 | −2.8 | 54.9 | 26 |
| LYS | CA | A | 286 | 9.5 | −2.3 | 56.2 | 31 |
| LYS | CB | A | 286 | 8.6 | −1.1 | 56.6 | 30 |
| LYS | CG | A | 286 | 8.8 | −0.6 | 58.0 | 34 |
| LYS | CD | A | 286 | 7.9 | 0.6 | 58.3 | 37 |
| LYS | CE | A | 286 | 8.2 | 1.1 | 59.7 | 42 |
| LYS | NZ | A | 286 | 7.2 | 2.2 | 60.1 | 45 |
| LYS | C | A | 286 | 9.4 | −3.4 | 57.2 | 30 |
| LYS | O | A | 286 | 10.2 | −3.5 | 58.1 | 34 |
| SER | N | A | 287 | 8.4 | −4.2 | 57.1 | 28 |
| SER | CA | A | 287 | 8.2 | −5.4 | 58.0 | 27 |
| SER | CB | A | 287 | 6.8 | −5.9 | 57.9 | 22 |
| SER | OG | A | 287 | 6.7 | −7.0 | 58.8 | 28 |
| SER | C | A | 287 | 9.3 | −6.4 | 57.7 | 25 |
| SER | O | A | 287 | 9.8 | −7.1 | 58.6 | 22 |
| ILE | N | A | 288 | 9.7 | −6.5 | 56.4 | 27 |
| ILE | CA | A | 288 | 10.7 | −7.5 | 56.1 | 27 |
| ILE | CB | A | 288 | 10.7 | −7.8 | 54.6 | 26 |
| ILE | CG2 | A | 288 | 11.8 | −8.8 | 54.2 | 23 |
| ILE | CG1 | A | 288 | 9.4 | −8.4 | 54.1 | 27 |
| ILE | CD1 | A | 288 | 9.3 | −8.7 | 52.7 | 24 |
| ILE | C | A | 288 | 12.1 | −7.1 | 56.6 | 29 |
| ILE | O | A | 288 | 12.9 | −8.0 | 56.9 | 30 |
| LYS | N | A | 289 | 12.3 | −5.8 | 56.6 | 28 |
| LYS | CA | A | 289 | 13.6 | −5.3 | 57.1 | 28 |
| LYS | CB | A | 289 | 13.9 | −3.9 | 56.8 | 23 |
| LYS | CG | A | 289 | 14.1 | −3.5 | 55.3 | 33 |
| LYS | CD | A | 289 | 14.4 | −2.0 | 55.2 | 33 |
| LYS | CE | A | 289 | 14.6 | −1.6 | 53.8 | 37 |
| LYS | NZ | A | 289 | 14.9 | −0.1 | 53.7 | 35 |
| LYS | C | A | 289 | 13.7 | −5.5 | 58.6 | 23 |
| LYS | O | A | 289 | 14.8 | −5.8 | 59.2 | 30 |
| ALA | N | A | 290 | 12.6 | −5.3 | 59.3 | 26 |
| ALA | CA | A | 290 | 12.6 | −5.5 | 60.8 | 23 |
| ALA | CB | A | 290 | 11.3 | −4.8 | 61.3 | 21 |
| ALA | C | A | 290 | 12.7 | −6.9 | 61.2 | 24 |
| ALA | O | A | 290 | 13.2 | −7.2 | 62.3 | 27 |
| ALA | N | A | 291 | 12.2 | −7.8 | 60.4 | 25 |
| ALA | CA | A | 291 | 12.2 | −9.2 | 60.8 | 25 |
| ALA | CB | A | 291 | 11.2 | −10.0 | 60.0 | 22 |
| ALA | C | A | 291 | 13.6 | −9.8 | 60.5 | 27 |
| ALA | O | A | 291 | 14.0 | −10.8 | 61.1 | 28 |
| SER | N | A | 292 | 14.4 | −9.2 | 59.6 | 29 |
| SER | CA | A | 292 | 15.7 | −9.7 | 59.3 | 31 |
| SER | CB | A | 292 | 15.9 | −9.8 | 57.7 | 34 |
| SER | OG | A | 292 | 15.7 | −8.6 | 57.1 | 36 |
| SER | C | A | 292 | 16.8 | −8.8 | 59.8 | 31 |
| SER | O | A | 292 | 17.9 | −8.9 | 59.5 | 31 |
| SER | N | A | 293 | 16.4 | −7.8 | 60.7 | 32 |
| SER | CA | A | 293 | 17.3 | −6.9 | 61.3 | 38 |
| SER | CB | A | 293 | 16.6 | −6.0 | 62.3 | 35 |
| SER | OG | A | 293 | 16.1 | −6.7 | 63.4 | 35 |
| SER | C | A | 293 | 18.6 | −7.4 | 61.8 | 42 |
| SER | O | A | 293 | 19.5 | −6.6 | 62.2 | 44 |
| THR | N | A | 294 | 18.8 | −8.7 | 62.0 | 45 |
| THR | CA | A | 294 | 20.0 | −9.3 | 62.5 | 47 |
| THR | CB | A | 294 | 19.9 | −10.8 | 62.9 | 47 |
| THR | OG1 | A | 294 | 19.5 | −11.6 | 61.8 | 51 |
| THR | CG2 | A | 294 | 18.9 | −11.0 | 64.1 | 49 |
| THR | C | A | 294 | 21.1 | −9.1 | 61.6 | 48 |
| THR | O | A | 294 | 22.3 | −9.2 | 61.9 | 51 |
| GLU | N | A | 295 | 20.8 | −8.7 | 60.3 | 50 |
| GLU | CA | A | 295 | 21.8 | −8.4 | 59.3 | 49 |
| GLU | CB | A | 295 | 22.1 | −9.6 | 58.4 | 48 |
| GLU | CG | A | 295 | 22.7 | −10.8 | 59.0 | 51 |
| GLU | CD | A | 295 | 23.0 | −11.9 | 58.0 | 51 |
| GLU | OE1 | A | 295 | 22.8 | −11.6 | 56.8 | 47 |
| GLU | OE2 | A | 295 | 23.3 | −13.0 | 58.4 | 53 |
| GLU | C | A | 295 | 21.3 | −7.2 | 58.5 | 50 |
| GLU | O | A | 295 | 20.1 | −7.1 | 58.1 | 50 |
| LYS | N | A | 296 | 22.2 | −6.3 | 58.3 | 53 |
| LYS | CA | A | 296 | 22.0 | −5.1 | 57.5 | 52 |
| LYS | CB | A | 296 | 22.6 | −3.8 | 58.1 | 55 |
| LYS | CG | A | 296 | 22.2 | −3.6 | 59.5 | 58 |
| LYS | CD | A | 296 | 20.6 | −3.4 | 59.5 | 58 |
| LYS | CE | A | 296 | 20.1 | −3.1 | 60.9 | 59 |
| LYS | NZ | A | 296 | 20.4 | −4.2 | 61.8 | 59 |
| LYS | C | A | 296 | 22.3 | −5.3 | 56.0 | 48 |
| LYS | O | A | 296 | 23.4 | −5.7 | 55.7 | 49 |
| PHE | N | A | 297 | 21.4 | −4.9 | 55.2 | 46 |
| PHE | CA | A | 297 | 21.5 | −5.0 | 53.7 | 47 |
| PHE | CB | A | 297 | 20.5 | −5.9 | 53.1 | 46 |
| PHE | CG | A | 297 | 20.4 | −7.3 | 53.7 | 47 |
| PHE | CD1 | A | 297 | 19.6 | −7.6 | 54.8 | 46 |
| PHE | CD2 | A | 297 | 21.2 | −8.3 | 53.2 | 49 |
| PHE | CE1 | A | 297 | 19.6 | −8.9 | 55.4 | 45 |
| PHE | CE2 | A | 297 | 21.3 | −9.6 | 53.8 | 49 |
| PHE | CZ | A | 297 | 20.4 | −9.9 | 54.9 | 48 |
| PHE | C | A | 297 | 21.5 | −3.7 | 53.0 | 48 |
| PHE | O | A | 297 | 20.6 | −2.8 | 53.3 | 42 |
| PRO | N | A | 298 | 22.5 | −3.4 | 52.1 | 51 |
| PRO | CD | A | 298 | 23.2 | −4.5 | 51.3 | 51 |
| PRO | CA | A | 298 | 22.5 | −2.2 | 51.4 | 52 |
| PRO | CB | A | 298 | 23.7 | −2.4 | 50.4 | 50 |
| PRO | CG | A | 298 | 23.4 | −3.7 | 49.9 | 52 |
| PRO | C | A | 298 | 21.2 | −1.8 | 50.7 | 54 |
| PRO | O | A | 298 | 20.6 | −2.7 | 50.0 | 55 |
| ASP | N | A | 299 | 20.8 | −0.6 | 50.8 | 57 |
| ASP | CA | A | 299 | 19.5 | −0.1 | 50.2 | 58 |
| ASP | CB | A | 299 | 19.5 | 1.5 | 50.3 | 63 |
| ASP | CG | A | 299 | 19.5 | 2.0 | 51.7 | 68 |
| ASP | OD1 | A | 299 | 19.6 | 1.2 | 52.7 | 71 |
| ASP | OD2 | A | 299 | 19.6 | 3.2 | 51.9 | 70 |
| ASP | C | A | 299 | 19.3 | −0.5 | 48.8 | 58 |
| ASP | O | A | 299 | 18.1 | −0.7 | 48.4 | 55 |
| GLY | N | A | 300 | 20.4 | −0.8 | 48.1 | 55 |
| GLY | CA | A | 300 | 20.2 | −1.2 | 46.7 | 52 |
| GLY | C | A | 300 | 19.6 | −2.6 | 46.7 | 48 |
| GLY | O | A | 300 | 18.8 | −2.9 | 45.8 | 45 |
| PHE | N | A | 301 | 19.9 | −3.4 | 47.7 | 44 |
| PHE | CA | A | 301 | 19.3 | −4.7 | 47.8 | 41 |
| PHE | CB | A | 301 | 19.8 | −5.4 | 49.1 | 39 |
| PHE | CG | A | 301 | 19.2 | −6.8 | 49.3 | 39 |
| PHE | CD1 | A | 301 | 19.5 | −7.9 | 48.5 | 38 |
| PHE | CD2 | A | 301 | 18.3 | −6.9 | 50.3 | 37 |
| PHE | CE1 | A | 301 | 18.9 | −9.1 | 48.7 | 39 |
| PHE | CE2 | A | 301 | 17.6 | −8.2 | 50.6 | 40 |
| PHE | CZ | A | 301 | 18.0 | −9.3 | 49.8 | 38 |
| PHE | C | A | 301 | 17.8 | −4.7 | 47.9 | 37 |
| PHE | O | A | 301 | 17.1 | −5.4 | 47.1 | 37 |
| TRP | N | A | 302 | 17.3 | −3.9 | 48.8 | 35 |
| TRP | CA | A | 302 | 15.8 | −3.8 | 49.0 | 34 |
| TRP | CB | A | 302 | 15.5 | −3.0 | 50.2 | 31 |
| TRP | CG | A | 302 | 16.0 | −3.7 | 51.5 | 35 |
| TRP | CD2 | A | 302 | 15.6 | −4.9 | 52.1 | 35 |
| TRP | CE2 | A | 302 | 16.4 | −5.2 | 53.2 | 34 |
| TRP | CE3 | A | 302 | 14.5 | −5.8 | 51.8 | 34 |
| TRP | CD1 | A | 302 | 17.1 | −3.3 | 52.2 | 34 |
| TRP | NE1 | A | 302 | 17.4 | −4.2 | 53.2 | 32 |
| TRP | CZ2 | A | 302 | 16.3 | −6.3 | 54.0 | 36 |
| TRP | CZ3 | A | 302 | 14.4 | −6.9 | 52.6 | 33 |
| TRP | CH2 | A | 302 | 15.2 | −7.2 | 53.7 | 37 |
| TRP | C | A | 302 | 15.1 | −3.2 | 47.7 | 36 |
| TRP | O | A | 302 | 13.9 | −3.2 | 47.6 | 38 |
| LEU | N | A | 303 | 15.9 | −2.6 | 46.8 | 37 |
| LEU | CA | A | 303 | 15.4 | −2.1 | 45.6 | 39 |
| LEU | CB | A | 303 | 16.1 | −0.8 | 45.1 | 40 |
| LEU | CG | A | 303 | 16.0 | 0.3 | 46.1 | 45 |
| LEU | CD1 | A | 303 | 16.8 | 1.5 | 45.7 | 43 |
| LEU | CD2 | A | 303 | 14.5 | 0.8 | 46.3 | 42 |
| LEU | C | A | 303 | 15.4 | −3.1 | 44.5 | 40 |
| LEU | O | A | 303 | 14.9 | −3.0 | 43.4 | 38 |
| GLY | N | A | 304 | 16.1 | −4.2 | 44.8 | 38 |
| GLY | CA | A | 304 | 16.3 | −5.3 | 43.9 | 45 |
| GLY | C | A | 304 | 17.3 | −5.0 | 42.8 | 49 |
| GLY | O | A | 304 | 17.3 | −5.6 | 41.7 | 50 |
| GLU | N | A | 305 | 18.2 | −4.1 | 43.1 | 52 |
| GLU | CA | A | 305 | 19.3 | −3.7 | 42.2 | 55 |
| GLU | CB | A | 305 | 19.4 | −2.2 | 42.2 | 54 |
| GLU | CG | A | 305 | 18.2 | −1.4 | 41.8 | 59 |
| GLU | CD | A | 305 | 18.4 | 0.1 | 41.9 | 62 |
| GLU | OE1 | A | 305 | 18.2 | 0.7 | 40.9 | 67 |
| GLU | OE2 | A | 305 | 18.8 | 0.6 | 42.9 | 62 |
| GLU | C | A | 305 | 20.6 | −4.4 | 42.4 | 57 |
| GLU | O | A | 305 | 21.4 | −4.5 | 41.5 | 58 |
| GLN | N | A | 306 | 20.9 | −4.7 | 43.7 | 54 |
| GLN | CA | A | 306 | 22.1 | −5.4 | 44.0 | 55 |
| GLN | CB | A | 306 | 23.1 | −4.4 | 44.7 | 58 |
| GLN | CG | A | 306 | 22.6 | −3.9 | 46.1 | 63 |
| GLN | CD | A | 306 | 23.7 | −3.0 | 46.7 | 66 |
| GLN | OE1 | A | 306 | 23.4 | −1.8 | 46.9 | 64 |
| GLN | NE2 | A | 306 | 24.8 | −3.5 | 46.9 | 65 |
| GLN | C | A | 306 | 22.0 | −6.7 | 44.8 | 54 |
| GLN | O | A | 306 | 21.2 | −6.8 | 45.7 | 55 |
| LEU | N | A | 307 | 22.8 | −7.7 | 44.3 | 53 |
| LEU | CA | A | 307 | 22.8 | −9.0 | 44.9 | 55 |
| LEU | CB | A | 307 | 23.2 | −10.1 | 43.9 | 59 |
| LEU | CG | A | 307 | 24.6 | −9.8 | 43.4 | 60 |
| LEU | CD1 | A | 307 | 25.7 | −9.8 | 44.4 | 61 |
| LEU | CD2 | A | 307 | 25.0 | −10.9 | 42.3 | 62 |
| LEU | C | A | 307 | 23.5 | −9.1 | 46.3 | 55 |
| LEU | O | A | 307 | 24.5 | −8.4 | 46.5 | 58 |
| VAL | N | A | 308 | 23.0 | −9.9 | 47.2 | 52 |
| VAL | CA | A | 308 | 23.6 | −10.2 | 48.5 | 46 |
| VAL | CB | A | 308 | 22.6 | −10.0 | 49.6 | 46 |
| VAL | CG1 | A | 308 | 23.2 | −10.4 | 51.0 | 46 |
| VAL | CG2 | A | 308 | 22.2 | −8.5 | 49.7 | 44 |
| VAL | C | A | 308 | 24.1 | −11.6 | 48.4 | 48 |
| VAL | O | A | 308 | 23.5 | −12.5 | 47.9 | 45 |
| CYS | N | A | 309 | 25.3 | −11.9 | 49.0 | 48 |
| CYS | CA | A | 309 | 26.0 | −13.2 | 48.9 | 46 |
| CYS | C | A | 309 | 26.3 | −13.8 | 50.3 | 44 |
| CYS | O | A | 309 | 26.4 | −13.1 | 51.3 | 42 |
| CYS | CB | A | 309 | 27.2 | −13.1 | 48.1 | 46 |
| CYS | SG | A | 309 | 27.0 | −12.4 | 46.4 | 49 |
| TRP | N | A | 310 | 26.4 | −15.1 | 50.3 | 45 |
| TRP | CA | A | 310 | 26.7 | −15.9 | 51.5 | 47 |
| TRP | CB | A | 310 | 25.5 | −16.3 | 52.3 | 44 |
| TRP | CG | A | 310 | 24.7 | −15.2 | 52.9 | 42 |
| TRP | CD2 | A | 310 | 23.5 | −14.7 | 52.5 | 41 |
| TRP | CE2 | A | 310 | 23.1 | −13.7 | 53.4 | 38 |
| TRP | CE3 | A | 310 | 22.6 | −14.9 | 51.4 | 40 |
| TRP | CD1 | A | 310 | 25.1 | −14.5 | 54.1 | 41 |
| TRP | NE1 | A | 310 | 24.1 | −13.6 | 54.4 | 38 |
| TRP | CZ2 | A | 310 | 21.9 | −12.9 | 53.3 | 38 |
| TRP | CZ3 | A | 310 | 21.4 | −14.2 | 51.3 | 39 |
| TRP | CH2 | A | 310 | 21.1 | −13.2 | 52.3 | 38 |
| TRP | C | A | 310 | 27.5 | −17.2 | 51.1 | 50 |
| TRP | O | A | 310 | 27.3 | −17.8 | 50.0 | 49 |
| GLN | N | A | 311 | 28.5 | −17.5 | 51.9 | 52 |
| GLN | CA | A | 311 | 29.4 | −18.7 | 51.6 | 55 |
| GLN | CB | A | 311 | 30.3 | −19.0 | 52.8 | 59 |
| GLN | CG | A | 311 | 31.1 | −20.3 | 52.7 | 68 |
| GLN | CD | A | 311 | 32.0 | −20.2 | 51.5 | 73 |
| GLN | OE1 | A | 311 | 32.1 | −19.3 | 50.7 | 77 |
| GLN | NE2 | A | 311 | 32.8 | −21.3 | 51.3 | 73 |
| GLN | C | A | 311 | 28.5 | −19.9 | 51.4 | 54 |
| GLN | O | A | 311 | 27.6 | −20.3 | 52.2 | 58 |
| ALA | N | A | 312 | 28.6 | −20.6 | 50.2 | 54 |
| ALA | CA | A | 312 | 27.9 | −21.7 | 49.8 | 55 |
| ALA | CB | A | 312 | 28.7 | −22.7 | 48.9 | 54 |
| ALA | C | A | 312 | 27.2 | −22.5 | 50.9 | 53 |
| ALA | O | A | 312 | 27.9 | −23.0 | 51.8 | 56 |
| GLY | N | A | 313 | 25.9 | −22.5 | 50.9 | 51 |
| GLY | CA | A | 313 | 25.1 | −23.3 | 52.0 | 43 |
| GLY | C | A | 313 | 24.9 | −22.6 | 53.3 | 42 |
| GLY | O | A | 313 | 24.3 | −23.1 | 54.2 | 45 |
| THR | N | A | 314 | 25.4 | −21.3 | 53.5 | 39 |
| THR | CA | A | 314 | 25.2 | −20.6 | 54.7 | 38 |
| THR | CB | A | 314 | 26.5 | −20.0 | 55.2 | 42 |
| THR | OG1 | A | 314 | 27.0 | −19.0 | 54.2 | 41 |
| THR | CG2 | A | 314 | 27.6 | −21.0 | 55.5 | 42 |
| THR | C | A | 314 | 24.1 | −19.6 | 54.7 | 35 |
| THR | O | A | 314 | 24.0 | −18.7 | 55.6 | 33 |
| THR | N | A | 315 | 23.3 | −19.6 | 53.6 | 34 |
| THR | CA | A | 315 | 22.2 | −18.6 | 53.5 | 30 |
| THR | CB | A | 315 | 21.4 | −18.8 | 52.2 | 33 |
| THR | OG1 | A | 315 | 22.3 | −18.8 | 51.1 | 33 |
| THR | CG2 | A | 315 | 20.3 | −17.8 | 52.1 | 29 |
| THR | C | A | 315 | 21.4 | −18.7 | 54.7 | 27 |
| THR | O | A | 315 | 20.8 | −19.7 | 55.0 | 28 |
| PRO | N | A | 316 | 21.3 | −17.6 | 55.5 | 27 |
| PRO | CD | A | 316 | 21.8 | −16.3 | 55.3 | 25 |
| PRO | CA | A | 316 | 20.5 | −17.7 | 56.8 | 27 |
| PRO | CB | A | 316 | 21.1 | −16.6 | 57.6 | 22 |
| PRO | CG | A | 316 | 21.1 | −15.5 | 56.5 | 26 |
| PRO | C | A | 316 | 19.0 | −17.5 | 56.5 | 27 |
| PRO | O | A | 316 | 18.4 | −16.5 | 56.8 | 25 |
| TRP | N | A | 317 | 18.4 | −18.6 | 55.9 | 25 |
| TRP | CA | A | 317 | 16.9 | −18.5 | 55.6 | 25 |
| TRP | CB | A | 317 | 16.5 | −19.9 | 55.0 | 23 |
| TRP | CG | A | 317 | 17.2 | −20.3 | 53.8 | 20 |
| TRP | CD2 | A | 317 | 17.1 | −19.7 | 52.5 | 21 |
| TRP | CE2 | A | 317 | 18.0 | −20.5 | 51.7 | 25 |
| TRP | CE3 | A | 317 | 16.4 | −18.6 | 52.0 | 18 |
| TRP | CD1 | A | 317 | 18.1 | −21.4 | 53.7 | 22 |
| TRP | NE1 | A | 317 | 18.6 | −21.5 | 52.5 | 27 |
| TRP | CZ2 | A | 317 | 18.2 | −20.2 | 50.3 | 23 |
| TRP | CZ3 | A | 317 | 16.6 | −18.3 | 50.6 | 18 |
| TRP | CH2 | A | 317 | 17.5 | −19.1 | 49.8 | 23 |
| TRP | C | A | 317 | 16.1 | −18.1 | 56.8 | 24 |
| TRP | O | A | 317 | 15.1 | −17.3 | 56.6 | 24 |
| ASN | N | A | 318 | 16.4 | −18.7 | 57.9 | 23 |
| ASN | CA | A | 318 | 15.6 | −18.4 | 59.1 | 23 |
| ASN | CB | A | 318 | 16.0 | −19.3 | 60.3 | 26 |
| ASN | CG | A | 318 | 17.4 | −18.9 | 60.8 | 29 |
| ASN | OD1 | A | 318 | 17.6 | −18.3 | 61.8 | 32 |
| ASN | ND2 | A | 318 | 18.4 | −19.3 | 60.0 | 27 |
| ASN | C | A | 318 | 15.5 | −17.0 | 59.6 | 24 |
| ASN | O | A | 318 | 14.6 | −16.7 | 60.4 | 24 |
| ILE | N | A | 319 | 16.4 | −16.1 | 59.2 | 26 |
| ILE | CA | A | 319 | 16.3 | −14.8 | 59.7 | 27 |
| ILE | CB | A | 319 | 17.7 | −14.1 | 59.8 | 26 |
| ILE | CG2 | A | 319 | 18.7 | −14.9 | 60.6 | 17 |
| ILE | CG1 | A | 319 | 18.4 | −13.8 | 58.5 | 28 |
| ILE | CD1 | A | 319 | 17.7 | −12.8 | 57.6 | 34 |
| ILE | C | A | 319 | 15.3 | −14.0 | 58.8 | 26 |
| ILE | O | A | 319 | 14.9 | −12.9 | 59.2 | 29 |
| PHE | N | A | 320 | 14.9 | −14.5 | 57.7 | 25 |
| PHE | CA | A | 320 | 13.9 | −14.0 | 56.8 | 26 |
| PHE | CB | A | 320 | 14.3 | −14.2 | 55.3 | 25 |
| PHE | CG | A | 320 | 15.5 | −13.5 | 54.9 | 30 |
| PHE | CD1 | A | 320 | 15.5 | −12.1 | 54.5 | 28 |
| PHE | CD2 | A | 320 | 16.8 | −14.1 | 54.9 | 29 |
| PHE | CE1 | A | 320 | 16.6 | −11.4 | 54.1 | 31 |
| PHE | CE2 | A | 320 | 17.9 | −13.5 | 54.5 | 29 |
| PHE | CZ | A | 320 | 17.9 | −12.1 | 54.1 | 33 |
| PHE | C | A | 320 | 12.6 | −14.5 | 57.2 | 20 |
| PHE | O | A | 320 | 12.4 | −15.7 | 57.4 | 19 |
| PRO | N | A | 321 | 11.5 | −13.6 | 57.2 | 21 |
| PRO | CD | A | 321 | 11.6 | −12.2 | 56.8 | 18 |
| PRO | CA | A | 321 | 10.2 | −14.0 | 57.4 | 21 |
| PRO | CB | A | 321 | 9.6 | −12.7 | 57.9 | 18 |
| PRO | CG | A | 321 | 10.1 | −11.8 | 56.8 | 20 |
| PRO | C | A | 321 | 9.4 | −14.6 | 56.3 | 24 |
| PRO | O | A | 321 | 9.8 | −14.5 | 55.1 | 23 |
| VAL | N | A | 322 | 8.4 | −15.4 | 56.6 | 22 |
| VAL | CA | A | 322 | 7.5 | −16.0 | 55.6 | 23 |
| VAL | CB | A | 322 | 6.8 | −17.3 | 56.1 | 27 |
| VAL | CG1 | A | 322 | 7.8 | −18.4 | 56.5 | 25 |
| VAL | CG2 | A | 322 | 5.9 | −16.9 | 57.3 | 20 |
| VAL | C | A | 322 | 6.5 | −15.0 | 55.2 | 25 |
| VAL | O | A | 322 | 6.2 | −14.1 | 56.0 | 24 |
| ILE | N | A | 323 | 6.0 | −15.0 | 54.0 | 23 |
| ILE | CA | A | 323 | 5.0 | −14.1 | 53.5 | 20 |
| ILE | CB | A | 323 | 5.5 | −13.3 | 52.3 | 20 |
| ILE | CG2 | A | 323 | 4.4 | −12.3 | 51.8 | 17 |
| ILE | CG1 | A | 323 | 6.8 | −12.5 | 52.6 | 15 |
| ILE | CD1 | A | 323 | 7.4 | −11.8 | 51.5 | 18 |
| ILE | C | A | 323 | 3.7 | −14.8 | 53.2 | 23 |
| ILE | O | A | 323 | 3.6 | −15.7 | 52.5 | 26 |
| SER | N | A | 324 | 2.6 | −14.3 | 53.9 | 20 |
| SER | CA | A | 324 | 1.3 | −14.9 | 53.7 | 22 |
| SER | CB | A | 324 | 0.7 | −15.5 | 55.0 | 23 |
| SER | OG | A | 324 | 1.6 | −16.5 | 55.5 | 28 |
| SER | C | A | 324 | 0.3 | −14.0 | 53.0 | 25 |
| SER | O | A | 324 | 0.1 | −12.9 | 53.4 | 26 |
| LEU | N | A | 325 | −0.4 | −14.6 | 52.0 | 25 |
| LEU | CA | A | 325 | −1.4 | −13.8 | 51.2 | 25 |
| LEU | CB | A | 325 | −1.1 | −13.7 | 49.8 | 28 |
| LEU | CG | A | 325 | 0.2 | −13.0 | 49.4 | 30 |
| LEU | CD1 | A | 325 | 0.4 | −13.0 | 47.9 | 32 |
| LEU | CD2 | A | 325 | 0.3 | −11.6 | 49.9 | 31 |
| LEU | C | A | 325 | −2.8 | −14.5 | 51.4 | 26 |
| LEU | O | A | 325 | −2.9 | −15.7 | 51.1 | 27 |
| TYR | N | A | 326 | −3.7 | −13.8 | 52.0 | 29 |
| TYR | CA | A | 326 | −5.1 | −14.4 | 52.1 | 30 |
| TYR | CB | A | 326 | −5.8 | −13.9 | 53.4 | 30 |
| TYR | CG | A | 326 | −5.2 | −14.3 | 54.7 | 29 |
| TYR | CD1 | A | 326 | −3.9 | −13.9 | 55.1 | 30 |
| TYR | CE1 | A | 326 | −3.4 | −14.3 | 56.3 | 31 |
| TYR | CD2 | A | 326 | −5.9 | −15.2 | 55.5 | 29 |
| TYR | CE2 | A | 326 | −5.3 | −15.7 | 56.7 | 33 |
| TYR | CZ | A | 326 | −4.1 | −15.2 | 57.1 | 32 |
| TYR | OH | A | 326 | −3.5 | −15.6 | 58.3 | 32 |
| TYR | C | A | 326 | −5.9 | −14.1 | 50.9 | 31 |
| TYR | O | A | 326 | −6.1 | −12.9 | 50.5 | 27 |
| LEU | N | A | 327 | −6.4 | −15.1 | 50.3 | 30 |
| LEU | CA | A | 327 | −7.2 | −15.1 | 49.1 | 33 |
| LEU | CB | A | 327 | −6.7 | −16.0 | 48.0 | 28 |
| LEU | CG | A | 327 | −5.2 | −15.7 | 47.5 | 31 |
| LEU | CD1 | A | 327 | −4.7 | −16.8 | 46.5 | 30 |
| LEU | CD2 | A | 327 | −5.1 | −14.3 | 46.9 | 30 |
| LEU | C | A | 327 | −8.6 | −15.4 | 49.4 | 34 |
| LEU | O | A | 327 | −8.9 | −16.3 | 50.1 | 36 |
| MET | N | A | 328 | −9.5 | −14.6 | 48.8 | 37 |
| MET | CA | A | 328 | −11.0 | −14.8 | 48.9 | 37 |
| MET | CB | A | 328 | −11.7 | −13.8 | 48.0 | 41 |
| MET | CG | A | 328 | −13.2 | −13.9 | 48.1 | 47 |
| MET | SD | A | 328 | −13.9 | −12.6 | 47.0 | 55 |
| MET | CE | A | 328 | −13.6 | −11.1 | 47.9 | 50 |
| MET | C | A | 328 | −11.3 | −16.3 | 48.5 | 35 |
| MET | O | A | 328 | −10.8 | −16.7 | 47.4 | 32 |
| GLY | N | A | 329 | −12.1 | −17.0 | 49.2 | 32 |
| GLY | CA | A | 329 | −12.5 | −18.3 | 48.9 | 38 |
| GLY | C | A | 329 | −13.8 | −18.4 | 48.1 | 38 |
| GLY | O | A | 329 | −14.4 | −17.3 | 47.9 | 35 |
| GLU | N | A | 330 | −14.2 | −19.6 | 47.7 | 41 |
| GLU | CA | A | 330 | −15.4 | −19.7 | 46.9 | 45 |
| GLU | CB | A | 330 | −15.5 | −20.9 | 46.0 | 42 |
| GLU | CG | A | 330 | −14.4 | −21.0 | 45.0 | 43 |
| GLU | CD | A | 330 | −14.6 | −22.2 | 44.1 | 46 |
| GLU | OE1 | A | 330 | −13.9 | −23.2 | 44.3 | 47 |
| GLU | OE2 | A | 330 | −15.5 | −22.2 | 43.3 | 48 |
| GLU | C | A | 330 | −16.7 | −19.6 | 47.9 | 47 |
| GLU | O | A | 330 | −17.8 | −19.5 | 47.4 | 49 |
| VAL | N | A | 331 | −16.4 | −19.8 | 49.1 | 48 |
| VAL | CA | A | 331 | −17.5 | −19.8 | 50.1 | 46 |
| VAL | CB | A | 331 | −17.3 | −20.9 | 51.2 | 46 |
| VAL | CG1 | A | 331 | −18.4 | −20.9 | 52.3 | 45 |
| VAL | CG2 | A | 331 | −17.2 | −22.3 | 50.6 | 46 |
| VAL | C | A | 331 | −17.6 | −18.5 | 50.8 | 51 |
| VAL | O | A | 331 | −16.6 | −17.9 | 51.2 | 50 |
| THR | N | A | 332 | −18.8 | −17.9 | 50.8 | 53 |
| THR | CA | A | 332 | −19.1 | −16.6 | 51.4 | 54 |
| THR | CB | A | 332 | −20.6 | −16.4 | 51.5 | 57 |
| THR | OG1 | A | 332 | −21.2 | −16.4 | 50.1 | 60 |
| THR | CG2 | A | 332 | −20.9 | −15.0 | 52.1 | 56 |
| THR | C | A | 332 | −18.5 | −16.5 | 52.8 | 52 |
| THR | O | A | 332 | −18.6 | −17.3 | 53.6 | 53 |
| ASN | N | A | 333 | −17.8 | −15.3 | 53.0 | 51 |
| ASN | CA | A | 333 | −17.1 | −15.1 | 54.3 | 53 |
| ASN | CB | A | 333 | −18.1 | −15.0 | 55.4 | 58 |
| ASN | CG | A | 333 | −19.1 | −13.8 | 55.2 | 63 |
| ASN | OD1 | A | 333 | −19.0 | −13.0 | 54.3 | 64 |
| ASN | ND2 | A | 333 | −20.1 | −13.7 | 56.1 | 64 |
| ASN | C | A | 333 | −16.0 | −16.0 | 54.6 | 50 |
| ASN | O | A | 333 | −15.5 | −16.0 | 55.8 | 55 |
| GLN | N | A | 334 | −15.6 | −16.8 | 53.7 | 45 |
| GLN | CA | A | 334 | −14.5 | −17.8 | 53.9 | 41 |
| GLN | CB | A | 334 | −15.0 | −19.2 | 53.7 | 41 |
| GLN | CG | A | 334 | −13.9 | −20.3 | 53.9 | 44 |
| GLN | CD | A | 334 | −14.6 | −21.6 | 53.7 | 47 |
| GLN | OE1 | A | 334 | −14.1 | −22.4 | 52.9 | 46 |
| GLN | NE2 | A | 334 | −15.6 | −22.0 | 54.5 | 46 |
| GLN | C | A | 334 | −13.2 | −17.5 | 53.0 | 41 |
| GLN | O | A | 334 | −13.4 | −17.1 | 51.9 | 37 |
| SER | N | A | 335 | −12.1 | −17.6 | 53.6 | 40 |
| SER | CA | A | 335 | −10.8 | −17.4 | 52.9 | 38 |
| SER | CB | A | 335 | −10.3 | −15.9 | 53.1 | 40 |
| SER | OG | A | 335 | −10.1 | −15.7 | 54.5 | 43 |
| SER | C | A | 335 | −9.7 | −18.4 | 53.3 | 37 |
| SER | O | A | 335 | −9.8 | −19.1 | 54.2 | 38 |
| PHE | N | A | 336 | −8.7 | −18.4 | 52.4 | 32 |
| PHE | CA | A | 336 | −7.5 | −19.3 | 52.7 | 30 |
| PHE | CB | A | 336 | −7.5 | −20.5 | 51.8 | 27 |
| PHE | CG | A | 336 | −7.4 | −20.2 | 50.3 | 27 |
| PHE | CD1 | A | 336 | −6.2 | −20.3 | 49.7 | 24 |
| PHE | CD2 | A | 336 | −8.5 | −19.8 | 49.6 | 24 |
| PHE | CE1 | A | 336 | −6.1 | −20.0 | 48.3 | 24 |
| PHE | CE2 | A | 336 | −8.4 | −19.5 | 48.2 | 24 |
| PHE | CZ | A | 336 | −7.2 | −19.6 | 47.5 | 20 |
| PHE | C | A | 336 | −6.3 | −18.4 | 52.5 | 31 |
| PHE | O | A | 336 | −6.3 | −17.3 | 52.0 | 35 |
| ARG | N | A | 337 | −5.1 | −19.0 | 52.9 | 28 |
| ARG | CA | A | 337 | −3.9 | −18.2 | 52.8 | 27 |
| ARG | CB | A | 337 | −3.4 | −17.7 | 54.1 | 25 |
| ARG | CG | A | 337 | −2.9 | −18.8 | 55.1 | 31 |
| ARG | CD | A | 337 | −2.4 | −18.2 | 56.4 | 30 |
| ARG | NE | A | 337 | −2.0 | −19.3 | 57.3 | 30 |
| ARG | CZ | A | 337 | −2.0 | −19.3 | 58.6 | 32 |
| ARG | NH1 | A | 337 | −2.4 | −18.2 | 59.3 | 28 |
| ARG | NH2 | A | 337 | −1.6 | −20.4 | 59.3 | 32 |
| ARG | C | A | 337 | −2.8 | −19.0 | 52.1 | 24 |
| ARG | O | A | 337 | −2.6 | −20.2 | 52.4 | 22 |
| ILE | N | A | 338 | −2.0 | −18.3 | 51.3 | 23 |
| ILE | CA | A | 338 | −0.8 | −19.0 | 50.7 | 23 |
| ILE | CB | A | 338 | −0.8 | −18.9 | 49.2 | 21 |
| ILE | CG2 | A | 338 | −2.0 | −19.6 | 48.6 | 23 |
| ILE | CG1 | A | 338 | −0.8 | −17.4 | 48.7 | 20 |
| ILE | CD1 | A | 338 | −0.7 | −17.2 | 47.2 | 23 |
| ILE | C | A | 338 | 0.4 | −18.4 | 51.4 | 24 |
| ILE | O | A | 338 | 0.5 | −17.2 | 51.5 | 24 |
| THR | N | A | 339 | 1.4 | −19.2 | 51.7 | 21 |
| THR | CA | A | 339 | 2.6 | −18.8 | 52.4 | 21 |
| THR | CB | A | 339 | 2.6 | −19.3 | 53.9 | 21 |
| THR | OG1 | A | 339 | 1.5 | −18.8 | 54.6 | 21 |
| THR | CG2 | A | 339 | 3.9 | −18.8 | 54.6 | 14 |
| THR | C | A | 339 | 3.9 | −19.2 | 51.7 | 22 |
| THR | O | A | 339 | 4.1 | −20.3 | 51.4 | 22 |
| ILE | N | A | 340 | 4.7 | −18.2 | 51.3 | 20 |
| ILE | CA | A | 340 | 5.9 | −18.4 | 50.6 | 18 |
| ILE | CB | A | 340 | 6.2 | −17.6 | 49.4 | 17 |
| ILE | CG2 | A | 340 | 5.1 | −18.0 | 48.3 | 20 |
| ILE | CG1 | A | 340 | 6.2 | −16.1 | 49.7 | 22 |
| ILE | CD1 | A | 340 | 6.4 | −15.2 | 48.5 | 18 |
| ILE | C | A | 340 | 7.1 | −18.2 | 51.6 | 21 |
| ILE | O | A | 340 | 7.0 | −17.6 | 52.6 | 21 |
| LEU | N | A | 341 | 8.2 | −18.8 | 51.3 | 20 |
| LEU | CA | A | 341 | 9.4 | −18.8 | 52.0 | 19 |
| LEU | CB | A | 341 | 10.1 | −20.2 | 52.1 | 18 |
| LEU | CG | A | 341 | 9.2 | −21.3 | 52.7 | 16 |
| LEU | CD1 | A | 341 | 9.9 | −22.6 | 52.5 | 10 |
| LEU | CD2 | A | 341 | 8.8 | −21.0 | 54.1 | 11 |
| LEU | C | A | 341 | 10.5 | −17.8 | 51.4 | 26 |
| LEU | O | A | 341 | 10.3 | −17.4 | 50.3 | 19 |
| PRO | N | A | 342 | 11.5 | −17.4 | 52.2 | 27 |
| PRO | CD | A | 342 | 12.0 | −17.8 | 53.5 | 24 |
| PRO | CA | A | 342 | 12.4 | −16.6 | 51.6 | 23 |
| PRO | CB | A | 342 | 13.3 | −16.1 | 52.7 | 30 |
| PRO | CG | A | 342 | 13.5 | −17.4 | 53.4 | 28 |
| PRO | C | A | 342 | 13.1 | −17.3 | 50.4 | 25 |
| PRO | O | A | 342 | 13.8 | −16.7 | 49.6 | 28 |
| GLN | N | A | 343 | 12.9 | −18.6 | 50.4 | 20 |
| GLN | CA | A | 343 | 13.5 | −19.4 | 49.2 | 20 |
| GLN | CB | A | 343 | 13.5 | −20.9 | 49.4 | 19 |
| GLN | CG | A | 343 | 14.4 | −21.6 | 50.4 | 21 |
| GLN | CD | A | 343 | 14.1 | −21.3 | 51.8 | 23 |
| GLN | OE1 | A | 343 | 13.2 | −20.5 | 52.1 | 23 |
| GLN | NE2 | A | 343 | 14.7 | −22.0 | 52.8 | 23 |
| GLN | C | A | 343 | 12.8 | −19.0 | 47.9 | 22 |
| GLN | O | A | 343 | 13.3 | −19.3 | 46.8 | 19 |
| GLN | N | A | 344 | 11.6 | −18.5 | 48.1 | 22 |
| GLN | CA | A | 344 | 10.8 | −18.0 | 46.9 | 23 |
| GLN | CB | A | 344 | 9.3 | −18.3 | 46.9 | 20 |
| GLN | CG | A | 344 | 8.8 | −19.8 | 46.9 | 24 |
| GLN | CD | A | 344 | 9.3 | −20.6 | 48.0 | 23 |
| GLN | OE1 | A | 344 | 10.1 | −21.5 | 47.8 | 23 |
| GLN | NE2 | A | 344 | 8.9 | −20.3 | 49.2 | 15 |
| GLN | C | A | 344 | 11.0 | −16.6 | 46.6 | 23 |
| GLN | O | A | 344 | 10.9 | −16.2 | 45.4 | 24 |
| TYR | N | A | 345 | 11.2 | −15.7 | 47.5 | 26 |
| TYR | CA | A | 345 | 11.4 | −14.3 | 47.2 | 25 |
| TYR | CB | A | 345 | 10.6 | −13.4 | 48.1 | 21 |
| TYR | CG | A | 345 | 10.8 | −13.4 | 49.6 | 21 |
| TYR | CD1 | A | 345 | 11.9 | −12.7 | 50.1 | 25 |
| TYR | CE1 | A | 345 | 12.1 | −12.6 | 51.5 | 24 |
| TYR | CD2 | A | 345 | 10.0 | −14.0 | 50.5 | 23 |
| TYR | CE2 | A | 345 | 10.2 | −14.0 | 51.9 | 23 |
| TYR | CZ | A | 345 | 11.3 | −13.2 | 52.4 | 26 |
| TYR | OH | A | 345 | 11.4 | −13.1 | 53.7 | 23 |
| TYR | C | A | 345 | 12.8 | −13.8 | 47.1 | 25 |
| TYR | O | A | 345 | 13.1 | −12.7 | 46.7 | 26 |
| LEU | N | A | 346 | 13.8 | −14.7 | 47.4 | 22 |
| LEU | CA | A | 346 | 15.2 | −14.3 | 47.2 | 26 |
| LEU | CB | A | 346 | 16.1 | −14.7 | 48.4 | 23 |
| LEU | CG | A | 346 | 15.7 | −13.9 | 49.7 | 28 |
| LEU | CD1 | A | 346 | 16.6 | −14.3 | 50.8 | 26 |
| LEU | CD2 | A | 346 | 15.8 | −12.4 | 49.5 | 28 |
| LEU | C | A | 346 | 15.6 | −15.1 | 45.9 | 24 |
| LEU | O | A | 346 | 15.9 | −16.3 | 46.0 | 27 |
| ARG | N | A | 347 | 15.6 | −14.4 | 44.8 | 26 |
| ARG | CA | A | 347 | 16.0 | −15.1 | 43.6 | 27 |
| ARG | CB | A | 347 | 15.3 | −14.4 | 42.4 | 24 |
| ARG | CG | A | 347 | 15.6 | −15.0 | 41.0 | 26 |
| ARG | CD | A | 347 | 14.9 | −14.3 | 39.9 | 30 |
| ARG | NE | A | 347 | 15.3 | −12.9 | 39.7 | 32 |
| ARG | CZ | A | 347 | 16.3 | −12.6 | 39.0 | 28 |
| ARG | NH1 | A | 347 | 17.1 | −13.5 | 38.4 | 23 |
| ARG | NH2 | A | 347 | 16.6 | −11.3 | 38.9 | 28 |
| ARG | C | A | 347 | 17.5 | −15.2 | 43.4 | 28 |
| ARG | O | A | 347 | 18.2 | −14.3 | 43.4 | 23 |
| PRO | N | A | 348 | 17.9 | −16.5 | 43.2 | 33 |
| PRO | CD | A | 348 | 17.1 | −17.7 | 43.0 | 33 |
| PRO | CA | A | 348 | 19.3 | −16.8 | 43.0 | 35 |
| PRO | CB | A | 348 | 19.4 | −18.3 | 43.3 | 35 |
| PRO | CG | A | 348 | 18.2 | −18.7 | 42.5 | 37 |
| PRO | C | A | 348 | 19.9 | −16.4 | 41.7 | 37 |
| PRO | O | A | 348 | 19.3 | −16.6 | 40.6 | 40 |
| VAL | N | A | 349 | 21.0 | −15.7 | 41.7 | 39 |
| VAL | CA | A | 349 | 21.7 | −15.2 | 40.5 | 44 |
| VAL | CB | A | 349 | 21.7 | −13.7 | 40.5 | 43 |
| VAL | CG1 | A | 349 | 20.3 | −13.2 | 40.4 | 41 |
| VAL | CG2 | A | 349 | 22.4 | −13.1 | 41.6 | 42 |
| VAL | C | A | 349 | 23.1 | −15.7 | 40.4 | 44 |
| VAL | O | A | 349 | 23.9 | −15.6 | 41.4 | 47 |
| SER | N | A | 355 | 31.4 | −23.5 | 43.2 | 90 |
| SER | CA | A | 355 | 30.6 | −22.3 | 42.8 | 90 |
| SER | CB | A | 355 | 29.1 | −22.6 | 42.6 | 91 |
| SER | OG | A | 355 | 28.5 | −23.1 | 43.8 | 92 |
| SER | C | A | 355 | 30.8 | −21.2 | 43.8 | 89 |
| SER | O | A | 355 | 30.2 | −20.1 | 43.6 | 91 |
| GLN | N | A | 356 | 31.7 | −21.3 | 44.8 | 84 |
| GLN | CA | A | 356 | 32.0 | −20.3 | 45.8 | 78 |
| GLN | CB | A | 356 | 32.8 | −19.1 | 45.2 | 77 |
| GLN | CG | A | 356 | 32.1 | −18.3 | 44.1 | 77 |
| GLN | CD | A | 356 | 32.9 | −17.2 | 43.7 | 77 |
| GLN | OE1 | A | 356 | 33.3 | −17.1 | 42.5 | 80 |
| GLN | NE2 | A | 356 | 33.2 | −16.3 | 44.6 | 77 |
| GLN | C | A | 356 | 30.8 | −19.8 | 46.7 | 74 |
| GLN | O | A | 356 | 30.4 | −20.5 | 47.5 | 74 |
| ASP | N | A | 357 | 30.4 | −18.6 | 46.4 | 69 |
| ASP | CA | A | 357 | 29.3 | −17.9 | 47.1 | 65 |
| ASP | CB | A | 357 | 29.7 | −16.5 | 47.4 | 66 |
| ASP | CG | A | 357 | 31.0 | −16.3 | 48.2 | 69 |
| ASP | OD1 | A | 357 | 30.9 | −15.7 | 49.3 | 66 |
| ASP | OD2 | A | 357 | 32.0 | −16.8 | 47.8 | 74 |
| ASP | C | A | 357 | 27.9 | −18.0 | 46.5 | 61 |
| ASP | O | A | 357 | 27.8 | −17.9 | 45.3 | 62 |
| ASP | N | A | 358 | 26.9 | −18.1 | 47.3 | 55 |
| ASP | CA | A | 358 | 25.5 | −18.1 | 46.9 | 50 |
| ASP | CB | A | 358 | 24.6 | −19.0 | 47.7 | 51 |
| ASP | CG | A | 358 | 25.1 | −20.5 | 47.6 | 52 |
| ASP | OD1 | A | 358 | 25.3 | −21.1 | 48.7 | 53 |
| ASP | OD2 | A | 358 | 25.2 | −21.0 | 46.5 | 54 |
| ASP | C | A | 358 | 25.0 | −16.7 | 46.9 | 44 |
| ASP | O | A | 358 | 25.0 | −16.1 | 48.0 | 40 |
| CYS | N | A | 359 | 24.6 | −16.1 | 45.8 | 42 |
| CYS | CA | A | 359 | 24.1 | −14.8 | 45.8 | 41 |
| CYS | C | A | 359 | 22.6 | −14.7 | 45.4 | 37 |
| CYS | O | A | 359 | 22.1 | −15.5 | 44.7 | 31 |
| CYS | CB | A | 359 | 24.9 | −14.0 | 44.8 | 44 |
| CYS | SG | A | 359 | 26.7 | −14.0 | 45.1 | 46 |
| TYR | N | A | 360 | 21.9 | −13.7 | 46.0 | 37 |
| TYR | CA | A | 360 | 20.5 | −13.5 | 45.7 | 34 |
| TYR | CB | A | 360 | 19.7 | −14.1 | 46.9 | 33 |
| TYR | CG | A | 360 | 20.0 | −15.5 | 47.3 | 32 |
| TYR | CD1 | A | 360 | 21.1 | −15.8 | 48.2 | 33 |
| TYR | CE1 | A | 360 | 21.5 | −17.0 | 48.5 | 29 |
| TYR | CD2 | A | 360 | 19.3 | −16.6 | 46.9 | 27 |
| TYR | CE2 | A | 360 | 19.6 | −17.9 | 47.2 | 27 |
| TYR | CZ | A | 360 | 20.7 | −18.1 | 48.1 | 30 |
| TYR | OH | A | 360 | 21.1 | −19.4 | 48.5 | 37 |
| TYR | C | A | 360 | 20.0 | −12.1 | 45.5 | 35 |
| TYR | O | A | 360 | 20.6 | −11.2 | 46.1 | 33 |
| LYS | N | A | 361 | 19.0 | −11.9 | 44.7 | 30 |
| LYS | CA | A | 361 | 18.4 | −10.6 | 44.5 | 33 |
| LYS | CB | A | 361 | 18.3 | −10.1 | 43.1 | 33 |
| LYS | CG | A | 361 | 19.6 | −9.8 | 42.4 | 39 |
| LYS | CD | A | 361 | 19.2 | −9.2 | 41.0 | 41 |
| LYS | CE | A | 361 | 20.5 | −8.9 | 40.2 | 41 |
| LYS | NZ | A | 361 | 20.1 | −8.3 | 38.9 | 41 |
| LYS | C | A | 361 | 17.0 | −10.6 | 45.2 | 32 |
| LYS | O | A | 361 | 16.3 | −11.6 | 45.1 | 30 |
| PHE | N | A | 362 | 16.6 | −9.5 | 45.8 | 28 |
| PHE | CA | A | 362 | 15.3 | −9.4 | 46.5 | 25 |
| PHE | CB | A | 362 | 15.3 | −8.1 | 47.3 | 25 |
| PHE | CG | A | 362 | 14.0 | −7.8 | 48.1 | 26 |
| PHE | CD1 | A | 362 | 13.6 | −8.7 | 49.1 | 23 |
| PHE | CD2 | A | 362 | 13.3 | −6.7 | 47.8 | 26 |
| PHE | CE1 | A | 362 | 12.4 | −8.5 | 49.8 | 24 |
| PHE | CE2 | A | 362 | 12.1 | −6.4 | 48.5 | 25 |
| PHE | CZ | A | 362 | 11.7 | −7.3 | 49.5 | 24 |
| PHE | C | A | 362 | 14.3 | −9.3 | 45.3 | 28 |
| PHE | O | A | 362 | 14.3 | −8.4 | 44.5 | 25 |
| ALA | N | A | 363 | 13.5 | −10.3 | 45.2 | 27 |
| ALA | CA | A | 363 | 12.5 | −10.4 | 44.1 | 27 |
| ALA | CB | A | 363 | 12.4 | −11.8 | 43.6 | 29 |
| ALA | C | A | 363 | 11.1 | −9.8 | 44.4 | 25 |
| ALA | O | A | 363 | 10.1 | −10.3 | 43.8 | 22 |
| ILE | N | A | 364 | 11.0 | −8.9 | 45.3 | 26 |
| ILE | CA | A | 364 | 9.8 | −8.2 | 45.6 | 25 |
| ILE | CB | A | 364 | 9.4 | −8.4 | 47.1 | 24 |
| ILE | CG2 | A | 364 | 8.0 | −7.6 | 47.3 | 21 |
| ILE | CG1 | A | 364 | 9.2 | −9.8 | 47.5 | 22 |
| ILE | CD1 | A | 364 | 8.8 | −10.1 | 48.9 | 23 |
| ILE | C | A | 364 | 9.9 | −6.8 | 45.2 | 25 |
| ILE | O | A | 364 | 10.9 | −6.1 | 45.6 | 30 |
| SER | N | A | 365 | 9.1 | −6.3 | 44.3 | 26 |
| SER | CA | A | 365 | 9.2 | −4.9 | 43.9 | 30 |
| SER | CB | A | 365 | 10.0 | −4.9 | 42.5 | 32 |
| SER | OG | A | 365 | 9.3 | −5.6 | 41.5 | 35 |
| SER | C | A | 365 | 7.9 | −4.1 | 43.8 | 33 |
| SER | O | A | 365 | 6.8 | −4.6 | 43.7 | 32 |
| GLN | N | A | 366 | 8.1 | −2.8 | 43.8 | 35 |
| GLN | CA | A | 366 | 7.1 | −1.8 | 43.7 | 36 |
| GLN | CB | A | 366 | 7.7 | −0.4 | 43.9 | 38 |
| GLN | CG | A | 366 | 6.7 | 0.7 | 43.9 | 46 |
| GLN | CD | A | 366 | 7.5 | 2.0 | 44.2 | 50 |
| GLN | OE1 | A | 366 | 7.3 | 2.6 | 45.2 | 53 |
| GLN | NE2 | A | 366 | 8.4 | 2.3 | 43.3 | 55 |
| GLN | C | A | 366 | 6.3 | −1.9 | 42.3 | 36 |
| GLN | O | A | 366 | 7.0 | −2.0 | 41.3 | 33 |
| SER | N | A | 367 | 5.0 | −1.7 | 42.4 | 34 |
| SER | CA | A | 367 | 4.2 | −1.7 | 41.2 | 34 |
| SER | CB | A | 367 | 3.4 | −3.0 | 41.1 | 35 |
| SER | OG | A | 367 | 2.5 | −2.9 | 40.0 | 30 |
| SER | C | A | 367 | 3.2 | −0.5 | 41.2 | 37 |
| SER | O | A | 367 | 2.7 | −0.1 | 42.2 | 34 |
| SER | N | A | 368 | 3.0 | 0.1 | 40.0 | 33 |
| SER | CA | A | 368 | 2.1 | 1.2 | 39.8 | 35 |
| SER | CB | A | 368 | 2.8 | 2.4 | 39.1 | 33 |
| SER | OG | A | 368 | 3.2 | 2.0 | 37.8 | 36 |
| SER | C | A | 368 | 0.9 | 0.8 | 39.1 | 33 |
| SER | O | A | 368 | 0.0 | 1.6 | 38.9 | 39 |
| THR | N | A | 369 | 0.8 | −0.5 | 38.8 | 33 |
| THR | CA | A | 369 | −0.3 | −1.1 | 38.1 | 32 |
| THR | CB | A | 369 | 0.0 | −1.6 | 36.7 | 35 |
| THR | OG1 | A | 369 | 1.0 | −2.6 | 36.8 | 34 |
| THR | CG2 | A | 369 | 0.5 | −0.4 | 35.8 | 35 |
| THR | C | A | 369 | −1.1 | −2.2 | 38.9 | 34 |
| THR | O | A | 369 | −1.8 | −3.0 | 38.4 | 31 |
| GLY | N | A | 370 | −0.8 | −2.3 | 40.2 | 28 |
| GLY | CA | A | 370 | −1.4 | −3.2 | 41.1 | 30 |
| GLY | C | A | 370 | −0.5 | −4.4 | 41.4 | 29 |
| GLY | O | A | 370 | 0.6 | −4.4 | 41.1 | 28 |
| THR | N | A | 371 | −1.1 | −5.4 | 42.2 | 29 |
| THR | CA | A | 371 | −0.4 | −6.5 | 42.6 | 27 |
| THR | CB | A | 371 | −1.1 | −7.3 | 43.8 | 28 |
| THR | OG1 | A | 371 | −1.1 | −6.4 | 44.9 | 27 |
| THR | CG2 | A | 371 | −0.4 | −8.6 | 44.1 | 24 |
| THR | C | A | 371 | −0.1 | −7.5 | 41.5 | 26 |
| THR | O | A | 371 | −1.0 | −7.9 | 40.7 | 30 |
| VAL | N | A | 372 | 1.1 | −8.0 | 41.4 | 27 |
| VAL | CA | A | 372 | 1.4 | −9.0 | 40.4 | 26 |
| VAL | CB | A | 372 | 2.4 | −8.5 | 39.3 | 27 |
| VAL | CG1 | A | 372 | 2.6 | −9.6 | 38.2 | 24 |
| VAL | CG2 | A | 372 | 1.9 | −7.2 | 38.7 | 25 |
| VAL | C | A | 372 | 2.0 | −10.3 | 41.1 | 27 |
| VAL | O | A | 372 | 3.1 | −10.2 | 41.6 | 25 |
| MET | N | A | 373 | 1.3 | −11.4 | 41.0 | 23 |
| MET | CA | A | 373 | 1.8 | −12.6 | 41.6 | 24 |
| MET | CB | A | 373 | 0.6 | −13.4 | 42.1 | 23 |
| MET | CG | A | 373 | −0.3 | −12.7 | 43.2 | 27 |
| MET | SD | A | 373 | −1.7 | −13.6 | 43.9 | 30 |
| MET | CE | A | 373 | −0.9 | −15.2 | 44.3 | 30 |
| MET | C | A | 373 | 2.5 | −13.3 | 40.5 | 24 |
| MET | O | A | 373 | 2.0 | −14.0 | 39.7 | 29 |
| GLY | N | A | 374 | 3.8 | −13.1 | 40.5 | 25 |
| GLY | CA | A | 374 | 4.7 | −13.7 | 39.5 | 23 |
| GLY | C | A | 374 | 5.3 | −15.0 | 39.9 | 24 |
| GLY | O | A | 374 | 4.9 | −15.7 | 40.8 | 19 |
| ALA | N | A | 375 | 6.4 | −15.3 | 39.2 | 24 |
| ALA | CA | A | 375 | 7.2 | −16.5 | 39.5 | 25 |
| ALA | CB | A | 375 | 8.5 | −16.5 | 38.6 | 26 |
| ALA | C | A | 375 | 7.5 | −16.6 | 41.0 | 27 |
| ALA | O | A | 375 | 7.6 | −17.7 | 41.5 | 31 |
| VAL | N | A | 376 | 7.7 | −15.5 | 41.6 | 28 |
| VAL | CA | A | 376 | 8.0 | −15.6 | 43.1 | 28 |
| VAL | CB | A | 376 | 8.3 | −14.2 | 43.7 | 26 |
| VAL | CG1 | A | 376 | 8.4 | −14.2 | 45.2 | 27 |
| VAL | CG2 | A | 376 | 9.5 | −13.5 | 43.1 | 26 |
| VAL | C | A | 376 | 7.0 | −16.3 | 43.8 | 27 |
| VAL | O | A | 376 | 7.3 | −17.2 | 44.6 | 36 |
| ILE | N | A | 377 | 5.7 | −16.0 | 43.5 | 25 |
| ILE | CA | A | 377 | 4.6 | −16.7 | 44.2 | 24 |
| ILE | CB | A | 377 | 3.3 | −15.9 | 44.2 | 24 |
| ILE | CG2 | A | 377 | 2.1 | −16.7 | 44.7 | 22 |
| ILE | CG1 | A | 377 | 3.4 | −14.6 | 44.9 | 28 |
| ILE | CD1 | A | 377 | 3.8 | −14.8 | 46.4 | 31 |
| ILE | C | A | 377 | 4.3 | −18.1 | 43.6 | 24 |
| ILE | O | A | 377 | 4.1 | −19.0 | 44.3 | 24 |
| MET | N | A | 378 | 4.4 | −18.1 | 42.2 | 22 |
| MET | CA | A | 378 | 4.1 | −19.4 | 41.6 | 25 |
| MET | CB | A | 378 | 3.9 | −19.2 | 40.1 | 23 |
| MET | CG | A | 378 | 2.7 | −18.3 | 39.8 | 23 |
| MET | SD | A | 378 | 2.4 | −18.0 | 38.0 | 29 |
| MET | CE | A | 378 | 1.6 | −19.6 | 37.6 | 18 |
| MET | C | A | 378 | 5.1 | −20.5 | 41.8 | 26 |
| MET | O | A | 378 | 4.7 | −21.7 | 41.7 | 31 |
| GLU | N | A | 379 | 6.4 | −20.2 | 42.0 | 25 |
| GLU | CA | A | 379 | 7.4 | −21.3 | 42.2 | 28 |
| GLU | CB | A | 379 | 8.8 | −20.7 | 42.1 | 26 |
| GLU | CG | A | 379 | 9.1 | −20.1 | 40.7 | 29 |
| GLU | CD | A | 379 | 10.5 | −19.6 | 40.7 | 30 |
| GLU | OE1 | A | 379 | 11.1 | −19.6 | 39.6 | 30 |
| GLU | OE2 | A | 379 | 11.1 | −19.3 | 41.7 | 35 |
| GLU | C | A | 379 | 7.2 | −22.1 | 43.5 | 26 |
| GLU | O | A | 379 | 7.9 | −23.0 | 43.8 | 28 |
| GLY | N | A | 380 | 6.2 | −21.7 | 44.3 | 24 |
| GLY | CA | A | 380 | 6.0 | −22.4 | 45.5 | 30 |
| GLY | C | A | 380 | 4.7 | −23.1 | 45.5 | 26 |
| GLY | O | A | 380 | 4.3 | −23.9 | 46.5 | 24 |
| PHE | N | A | 381 | 3.9 | −23.0 | 44.5 | 28 |
| PHE | CA | A | 381 | 2.6 | −23.6 | 44.4 | 27 |
| PHE | CB | A | 381 | 1.5 | −22.5 | 44.7 | 25 |
| PHE | CG | A | 381 | 1.7 | −21.9 | 46.1 | 27 |
| PHE | CD1 | A | 381 | 2.2 | −20.6 | 46.1 | 26 |
| PHE | CD2 | A | 381 | 1.5 | −22.6 | 47.3 | 26 |
| PHE | CE1 | A | 381 | 2.5 | −19.9 | 47.3 | 27 |
| PHE | CE2 | A | 381 | 1.8 | −21.9 | 48.5 | 28 |
| PHE | CZ | A | 381 | 2.3 | −20.6 | 48.5 | 27 |
| PHE | C | A | 381 | 2.2 | −24.2 | 43.1 | 28 |
| PHE | O | A | 381 | 2.6 | −23.7 | 42.0 | 25 |
| TYR | N | A | 382 | 1.4 | −25.3 | 43.1 | 28 |
| TYR | CA | A | 382 | 0.9 | −25.9 | 41.9 | 26 |
| TYR | CB | A | 382 | 0.5 | −27.3 | 42.1 | 25 |
| TYR | CG | A | 382 | 0.0 | −28.0 | 40.9 | 29 |
| TYR | CD1 | A | 382 | 0.7 | −27.9 | 39.7 | 30 |
| TYR | CE1 | A | 382 | 0.2 | −28.6 | 38.5 | 30 |
| TYR | CD2 | A | 382 | −1.2 | −28.7 | 40.9 | 28 |
| TYR | CE2 | A | 382 | −1.7 | −29.3 | 39.8 | 24 |
| TYR | CZ | A | 382 | −1.0 | −29.3 | 38.6 | 29 |
| TYR | OH | A | 382 | −1.5 | −29.9 | 37.5 | 27 |
| TYR | C | A | 382 | −0.3 | −25.0 | 41.7 | 23 |
| TYR | O | A | 382 | −1.2 | −24.9 | 42.5 | 23 |
| VAL | N | A | 383 | −0.3 | −24.3 | 40.5 | 20 |
| VAL | CA | A | 383 | −1.4 | −23.4 | 40.2 | 22 |
| VAL | CB | A | 383 | −0.9 | −22.0 | 39.9 | 22 |
| VAL | CG1 | A | 383 | −2.0 | −21.0 | 39.7 | 20 |
| VAL | CG2 | A | 383 | 0.0 | −21.5 | 41.0 | 19 |
| VAL | C | A | 383 | −2.4 | −23.8 | 39.2 | 27 |
| VAL | O | A | 383 | −2.0 | −24.2 | 38.0 | 28 |
| VAL | N | A | 384 | −3.7 | −23.9 | 39.5 | 25 |
| VAL | CA | A | 384 | −4.7 | −24.4 | 38.6 | 25 |
| VAL | CB | A | 384 | −5.6 | −25.5 | 39.2 | 25 |
| VAL | CG1 | A | 384 | −6.6 | −26.0 | 38.2 | 24 |
| VAL | CG2 | A | 384 | −4.7 | −26.6 | 39.8 | 22 |
| VAL | C | A | 384 | −5.6 | −23.3 | 38.0 | 26 |
| VAL | O | A | 384 | −6.4 | −22.7 | 38.7 | 29 |
| PHE | N | A | 385 | −5.4 | −23.0 | 36.7 | 25 |
| PHE | CA | A | 385 | −6.3 | −22.0 | 36.1 | 28 |
| PHE | CB | A | 385 | −5.5 | −21.3 | 34.9 | 26 |
| PHE | CG | A | 385 | −4.3 | −20.6 | 35.4 | 28 |
| PHE | CD1 | A | 385 | −4.4 | −19.2 | 35.6 | 25 |
| PHE | CD2 | A | 385 | −3.2 | −21.2 | 35.7 | 24 |
| PHE | CE1 | A | 385 | −3.3 | −18.5 | 36.0 | 26 |
| PHE | CE2 | A | 385 | −2.0 | −20.5 | 36.2 | 25 |
| PHE | CZ | A | 385 | −2.1 | −19.1 | 36.4 | 24 |
| PHE | C | A | 385 | −7.5 | −22.8 | 35.6 | 31 |
| PHE | O | A | 385 | −7.5 | −23.3 | 34.5 | 31 |
| ASP | N | A | 386 | −8.5 | −22.8 | 36.4 | 30 |
| ASP | CA | A | 386 | −9.8 | −23.5 | 36.1 | 32 |
| ASP | CB | A | 386 | −10.4 | −24.1 | 37.4 | 36 |
| ASP | CG | A | 386 | −11.6 | −24.9 | 37.1 | 37 |
| ASP | OD1 | A | 386 | −12.1 | −24.9 | 36.0 | 41 |
| ASP | OD2 | A | 386 | −12.1 | −25.5 | 38.1 | 39 |
| ASP | C | A | 386 | −10.7 | −22.5 | 35.4 | 30 |
| ASP | O | A | 386 | −11.5 | −21.8 | 36.1 | 29 |
| ARG | N | A | 387 | −10.5 | −22.4 | 34.1 | 30 |
| ARG | CA | A | 387 | −11.3 | −21.5 | 33.3 | 33 |
| ARG | CB | A | 387 | −10.7 | −21.5 | 31.9 | 35 |
| ARG | CG | A | 387 | −9.3 | −20.9 | 31.8 | 34 |
| ARG | CD | A | 387 | −8.7 | −21.0 | 30.4 | 37 |
| ARG | NE | A | 387 | −9.5 | −20.3 | 29.4 | 41 |
| ARG | CZ | A | 387 | −9.0 | −19.4 | 28.6 | 43 |
| ARG | NH1 | A | 387 | −7.7 | −19.0 | 28.7 | 44 |
| ARG | NH2 | A | 387 | −9.8 | −18.9 | 27.6 | 47 |
| ARG | C | A | 387 | −12.8 | −21.8 | 33.2 | 33 |
| ARG | O | A | 387 | −13.6 | −21.0 | 33.1 | 35 |
| ALA | N | A | 388 | −13.0 | −23.1 | 33.3 | 34 |
| ALA | CA | A | 388 | −14.4 | −23.6 | 33.2 | 38 |
| ALA | CB | A | 388 | −14.4 | −25.1 | 32.9 | 36 |
| ALA | C | A | 388 | −15.2 | −23.4 | 34.5 | 39 |
| ALA | O | A | 388 | −16.4 | −23.5 | 34.4 | 40 |
| ARG | N | A | 389 | −14.5 | −23.0 | 35.5 | 38 |
| ARG | CA | A | 389 | −15.2 | −22.7 | 36.8 | 38 |
| ARG | CB | A | 389 | −15.0 | −23.8 | 37.8 | 38 |
| ARG | CG | A | 389 | −15.4 | −25.2 | 37.3 | 37 |
| ARG | CD | A | 389 | −15.3 | −26.3 | 38.3 | 38 |
| ARG | NE | A | 389 | −16.0 | −26.0 | 39.5 | 46 |
| ARG | CZ | A | 389 | −16.3 | −27.0 | 40.4 | 46 |
| ARG | NH1 | A | 389 | −15.8 | −28.2 | 40.3 | 42 |
| ARG | NH2 | A | 389 | −17.0 | −26.6 | 41.5 | 47 |
| ARG | C | A | 389 | −14.9 | −21.3 | 37.3 | 37 |
| ARG | O | A | 389 | −15.3 | −20.9 | 38.4 | 39 |
| LYS | N | A | 390 | −14.2 | −20.6 | 36.5 | 36 |
| LYS | CA | A | 390 | −13.8 | −19.2 | 36.8 | 42 |
| LYS | CB | A | 390 | −15.1 | −18.3 | 36.5 | 43 |
| LYS | CG | A | 390 | −14.9 | −16.8 | 36.7 | 49 |
| LYS | CD | A | 390 | −16.2 | −16.1 | 36.4 | 51 |
| LYS | CE | A | 390 | −16.1 | −14.6 | 36.6 | 54 |
| LYS | NZ | A | 390 | −17.4 | −13.8 | 36.3 | 51 |
| LYS | C | A | 390 | −13.2 | −19.1 | 38.2 | 39 |
| LYS | O | A | 390 | −13.6 | −18.2 | 39.0 | 41 |
| ARG | N | A | 391 | −12.3 | −19.9 | 38.4 | 33 |
| ARG | CA | A | 391 | −11.6 | −20.0 | 39.7 | 31 |
| ARG | CB | A | 391 | −12.3 | −20.9 | 40.7 | 26 |
| ARG | CG | A | 391 | −12.3 | −22.4 | 40.2 | 27 |
| ARG | CD | A | 391 | −13.0 | −23.3 | 41.2 | 24 |
| ARG | NE | A | 391 | −12.9 | −24.7 | 40.7 | 23 |
| ARG | CZ | A | 391 | −13.1 | −25.8 | 41.4 | 26 |
| ARG | NH1 | A | 391 | −13.4 | −25.6 | 42.7 | 26 |
| ARG | NH2 | A | 391 | −13.0 | −27.0 | 40.9 | 23 |
| ARG | C | A | 391 | −10.1 | −20.4 | 39.5 | 34 |
| ARG | O | A | 391 | −9.8 | −21.1 | 38.6 | 31 |
| ILE | N | A | 392 | −9.2 | −20.0 | 40.4 | 35 |
| ILE | CA | A | 392 | −7.8 | −20.3 | 40.4 | 31 |
| ILE | CB | A | 392 | −6.9 | −19.1 | 40.2 | 31 |
| ILE | CG2 | A | 392 | −5.4 | −19.5 | 40.4 | 32 |
| ILE | CG1 | A | 392 | −7.2 | −18.4 | 38.9 | 30 |
| ILE | CD1 | A | 392 | −6.3 | −17.2 | 38.6 | 33 |
| ILE | C | A | 392 | −7.5 | −21.1 | 41.6 | 33 |
| ILE | O | A | 392 | −7.7 | −20.6 | 42.8 | 36 |
| GLY | N | A | 393 | −6.9 | −22.3 | 41.4 | 29 |
| GLY | CA | A | 393 | −6.5 | −23.1 | 42.6 | 24 |
| GLY | C | A | 393 | −5.1 | −23.1 | 42.9 | 29 |
| GLY | O | A | 393 | −4.2 | −23.0 | 42.1 | 33 |
| PHE | N | A | 394 | −4.8 | −23.2 | 44.2 | 27 |
| PHE | CA | A | 394 | −3.4 | −23.2 | 44.8 | 27 |
| PHE | CB | A | 394 | −3.1 | −22.0 | 45.6 | 23 |
| PHE | CG | A | 394 | −3.2 | −20.7 | 44.8 | 23 |
| PHE | CD1 | A | 394 | −4.4 | −20.1 | 44.4 | 24 |
| PHE | CD2 | A | 394 | −2.0 | −20.1 | 44.5 | 22 |
| PHE | CE1 | A | 394 | −4.4 | −18.9 | 43.7 | 19 |
| PHE | CE2 | A | 394 | −2.0 | −18.8 | 43.8 | 22 |
| PHE | CZ | A | 394 | −3.2 | −18.3 | 43.4 | 20 |
| PHE | C | A | 394 | −3.2 | −24.5 | 45.7 | 27 |
| PHE | O | A | 394 | −4.1 | −24.9 | 46.4 | 27 |
| ALA | N | A | 395 | −2.0 | −25.0 | 45.6 | 28 |
| ALA | CA | A | 395 | −1.6 | −26.2 | 46.4 | 28 |
| ALA | CB | A | 395 | −2.1 | −27.5 | 45.8 | 26 |
| ALA | C | A | 395 | −0.1 | −26.2 | 46.6 | 32 |
| ALA | O | A | 395 | 0.6 | −25.7 | 45.7 | 31 |
| VAL | N | A | 396 | 0.3 | −26.7 | 47.7 | 30 |
| VAL | CA | A | 396 | 1.8 | −26.8 | 47.9 | 29 |
| VAL | CB | A | 396 | 2.1 | −27.4 | 49.3 | 31 |
| VAL | CG1 | A | 396 | 3.6 | −27.5 | 49.5 | 29 |
| VAL | CG2 | A | 396 | 1.5 | −26.7 | 50.4 | 29 |
| VAL | C | A | 396 | 2.5 | −27.5 | 46.8 | 29 |
| VAL | O | A | 396 | 2.1 | −28.6 | 46.5 | 26 |
| SER | N | A | 397 | 3.4 | −26.8 | 46.1 | 33 |
| SER | CA | A | 397 | 4.1 | −27.4 | 45.0 | 28 |
| SER | CB | A | 397 | 4.9 | −26.3 | 44.3 | 27 |
| SER | OG | A | 397 | 5.6 | −26.9 | 43.2 | 23 |
| SER | C | A | 397 | 5.1 | −28.5 | 45.5 | 30 |
| SER | O | A | 397 | 5.8 | −28.3 | 46.4 | 26 |
| ALA | N | A | 398 | 5.0 | −29.6 | 44.8 | 30 |
| ALA | CA | A | 398 | 5.9 | −30.7 | 45.1 | 31 |
| ALA | CB | A | 398 | 5.5 | −32.0 | 44.3 | 29 |
| ALA | C | A | 398 | 7.4 | −30.4 | 44.8 | 33 |
| ALA | O | A | 398 | 8.3 | −31.1 | 45.2 | 34 |
| CYS | N | A | 399 | 7.6 | −29.3 | 44.0 | 31 |
| CYS | CA | A | 399 | 9.0 | −28.9 | 43.7 | 33 |
| CYS | C | A | 399 | 9.5 | −27.6 | 44.3 | 34 |
| CYS | O | A | 399 | 10.5 | −27.1 | 44.0 | 32 |
| CYS | CB | A | 399 | 9.2 | −28.9 | 42.2 | 35 |
| CYS | SG | A | 399 | 8.3 | −27.6 | 41.2 | 38 |
| HIS | N | A | 400 | 8.7 | −27.0 | 45.2 | 33 |
| HIS | CA | A | 400 | 9.1 | −25.7 | 45.7 | 31 |
| HIS | CB | A | 400 | 8.0 | −25.0 | 46.6 | 32 |
| HIS | CG | A | 400 | 7.7 | −25.6 | 47.9 | 31 |
| HIS | CD2 | A | 400 | 7.9 | −25.2 | 49.1 | 29 |
| HIS | ND1 | A | 400 | 7.1 | −26.9 | 48.0 | 30 |
| HIS | CE1 | A | 400 | 7.0 | −27.1 | 49.3 | 32 |
| HIS | NE2 | A | 400 | 7.5 | −26.1 | 50.0 | 29 |
| HIS | C | A | 400 | 10.3 | −25.9 | 46.6 | 29 |
| HIS | O | A | 400 | 10.5 | −26.9 | 47.2 | 26 |
| VAL | N | A | 401 | 11.2 | −24.9 | 46.5 | 27 |
| VAL | CA | A | 401 | 12.4 | −24.9 | 47.3 | 27 |
| VAL | CB | A | 401 | 13.5 | −24.0 | 46.7 | 24 |
| VAL | CG1 | A | 401 | 14.7 | −24.0 | 47.6 | 27 |
| VAL | CG2 | A | 401 | 13.8 | −24.3 | 45.3 | 25 |
| VAL | C | A | 401 | 12.1 | −24.6 | 48.8 | 27 |
| VAL | O | A | 401 | 11.4 | −23.6 | 49.1 | 28 |
| HIS | N | A | 402 | 12.7 | −25.4 | 49.7 | 28 |
| HIS | CA | A | 402 | 12.5 | −25.1 | 51.1 | 29 |
| HIS | CB | A | 402 | 11.2 | −25.6 | 51.6 | 32 |
| HIS | CG | A | 402 | 11.0 | −27.1 | 51.5 | 36 |
| HIS | CD2 | A | 402 | 11.1 | −28.1 | 52.4 | 37 |
| HIS | ND1 | A | 402 | 10.6 | −27.7 | 50.3 | 38 |
| HIS | CE1 | A | 402 | 10.5 | −29.0 | 50.5 | 36 |
| HIS | NE2 | A | 402 | 10.8 | −29.2 | 51.8 | 40 |
| HIS | C | A | 402 | 13.7 | −25.7 | 51.9 | 27 |
| HIS | O | A | 402 | 14.6 | −26.2 | 51.3 | 31 |
| ASP | N | A | 403 | 13.5 | −25.7 | 53.2 | 29 |
| ASP | CA | A | 403 | 14.6 | −26.2 | 54.1 | 28 |
| ASP | CB | A | 403 | 15.3 | −25.2 | 54.8 | 29 |
| ASP | CG | A | 403 | 14.4 | −24.3 | 55.7 | 32 |
| ASP | OD1 | A | 403 | 13.6 | −24.8 | 56.4 | 32 |
| ASP | OD2 | A | 403 | 14.6 | −23.1 | 55.7 | 35 |
| ASP | C | A | 403 | 13.9 | −27.2 | 55.0 | 28 |
| ASP | O | A | 403 | 12.8 | −27.6 | 54.8 | 27 |
| GLU | N | A | 404 | 14.6 | −27.7 | 56.0 | 30 |
| GLU | CA | A | 404 | 14.1 | −28.7 | 56.9 | 33 |
| GLU | CB | A | 404 | 15.1 | −29.6 | 57.5 | 39 |
| GLU | CG | A | 404 | 16.3 | −29.1 | 58.4 | 45 |
| GLU | CD | A | 404 | 17.2 | −28.1 | 57.6 | 50 |
| GLU | OE1 | A | 404 | 17.0 | −27.8 | 56.4 | 51 |
| GLU | OE2 | A | 404 | 18.3 | −27.8 | 58.2 | 48 |
| GLU | C | A | 404 | 13.2 | −28.1 | 58.0 | 33 |
| GLU | O | A | 404 | 12.5 | −28.9 | 58.7 | 33 |
| PHE | N | A | 405 | 13.1 | −26.8 | 58.2 | 28 |
| PHE | CA | A | 405 | 12.3 | −26.2 | 59.2 | 25 |
| PHE | CB | A | 405 | 13.2 | −25.2 | 60.0 | 26 |
| PHE | CG | A | 405 | 14.4 | −25.8 | 60.6 | 30 |
| PHE | CD1 | A | 405 | 15.6 | −25.6 | 60.0 | 30 |
| PHE | CD2 | A | 405 | 14.3 | −26.7 | 61.7 | 29 |
| PHE | CE1 | A | 405 | 16.8 | −26.3 | 60.5 | 32 |
| PHE | CE2 | A | 405 | 15.4 | −27.3 | 62.2 | 32 |
| PHE | CZ | A | 405 | 16.7 | −27.1 | 61.6 | 30 |
| PHE | C | A | 405 | 11.0 | −25.5 | 58.8 | 27 |
| PHE | O | A | 405 | 10.1 | −25.4 | 59.5 | 29 |
| ARG | N | A | 406 | 11.0 | −25.1 | 57.5 | 25 |
| ARG | CA | A | 406 | 9.8 | −24.4 | 56.9 | 24 |
| ARG | CB | A | 406 | 9.9 | −22.9 | 57.0 | 23 |
| ARG | CG | A | 406 | 10.0 | −22.3 | 58.4 | 24 |
| ARG | CD | A | 406 | 10.0 | −20.8 | 58.3 | 23 |
| ARG | NE | A | 406 | 11.1 | −20.2 | 57.5 | 24 |
| ARG | CZ | A | 406 | 11.4 | −18.9 | 57.6 | 27 |
| ARG | NH1 | A | 406 | 10.9 | −18.1 | 58.5 | 22 |
| ARG | NH2 | A | 406 | 12.4 | −18.4 | 56.8 | 24 |
| ARG | C | A | 406 | 9.5 | −24.8 | 55.5 | 30 |
| ARG | O | A | 406 | 10.4 | −25.1 | 54.7 | 29 |
| THR | N | A | 407 | 8.2 | −24.9 | 55.2 | 28 |
| THR | CA | A | 407 | 7.7 | −25.3 | 53.9 | 28 |
| THR | CB | A | 407 | 7.2 | −26.7 | 53.9 | 27 |
| THR | OG1 | A | 407 | 6.6 | −27.0 | 52.6 | 36 |
| THR | CG2 | A | 407 | 6.1 | −26.8 | 55.0 | 29 |
| THR | C | A | 407 | 6.6 | −24.3 | 53.5 | 29 |
| THR | O | A | 407 | 5.9 | −23.7 | 54.3 | 28 |
| ALA | N | A | 408 | 6.5 | −24.1 | 52.2 | 26 |
| ALA | CA | A | 408 | 5.4 | −23.2 | 51.7 | 28 |
| ALA | CB | A | 408 | 5.5 | −23.1 | 50.1 | 26 |
| ALA | C | A | 408 | 4.1 | −23.9 | 52.1 | 27 |
| ALA | O | A | 408 | 4.1 | −25.1 | 52.2 | 29 |
| ALA | N | A | 409 | 3.1 | −23.1 | 52.3 | 26 |
| ALA | CA | A | 409 | 1.8 | −23.6 | 52.7 | 23 |
| ALA | CB | A | 409 | 1.7 | −23.5 | 54.2 | 23 |
| ALA | C | A | 409 | 0.6 | −23.0 | 52.1 | 27 |
| ALA | O | A | 409 | 0.6 | −21.8 | 51.6 | 21 |
| VAL | N | A | 410 | −0.5 | −23.8 | 52.1 | 24 |
| VAL | CA | A | 410 | −1.8 | −23.4 | 51.6 | 27 |
| VAL | CB | A | 410 | −2.2 | −24.0 | 50.2 | 26 |
| VAL | CG1 | A | 410 | −3.6 | −23.5 | 49.8 | 25 |
| VAL | CG2 | A | 410 | −1.2 | −23.8 | 49.2 | 24 |
| VAL | C | A | 410 | −2.7 | −23.8 | 52.7 | 29 |
| VAL | O | A | 410 | −2.9 | −25.0 | 53.0 | 25 |
| GLU | N | A | 411 | −3.3 | −22.9 | 53.4 | 28 |
| GLU | CA | A | 411 | −4.1 | −23.2 | 54.6 | 30 |
| GLU | CB | A | 411 | −3.3 | −22.9 | 55.8 | 32 |
| GLU | CG | A | 411 | −2.0 | −23.7 | 55.8 | 39 |
| GLU | CD | A | 411 | −1.2 | −23.3 | 57.0 | 44 |
| GLU | OE1 | A | 411 | −0.9 | −24.2 | 57.8 | 50 |
| GLU | OE2 | A | 411 | −0.9 | −22.1 | 57.2 | 44 |
| GLU | C | A | 411 | −5.5 | −22.4 | 54.7 | 30 |
| GLU | O | A | 411 | −5.6 | −21.3 | 54.2 | 23 |
| GLY | N | A | 412 | −6.5 | −23.2 | 55.2 | 32 |
| GLY | CA | A | 412 | −7.8 | −22.6 | 55.3 | 32 |
| GLY | C | A | 412 | −8.7 | −23.5 | 56.1 | 28 |
| GLY | O | A | 412 | −8.3 | −24.6 | 56.4 | 29 |
| PRO | N | A | 413 | −9.9 | −23.1 | 56.4 | 32 |
| PRO | CD | A | 413 | −11.0 | −24.0 | 56.9 | 32 |
| PRO | CA | A | 413 | −10.5 | −21.8 | 56.1 | 33 |
| PRO | CB | A | 413 | −11.9 | −22.2 | 55.7 | 33 |
| PRO | CG | A | 413 | −12.3 | −23.1 | 56.9 | 34 |
| PRO | C | A | 413 | −10.5 | −20.8 | 57.3 | 34 |
| PRO | O | A | 413 | −10.6 | −21.1 | 58.4 | 38 |
| PHE | N | A | 414 | −10.4 | −19.5 | 56.9 | 36 |
| PHE | CA | A | 414 | −10.4 | −18.4 | 57.9 | 41 |
| PHE | CB | A | 414 | −9.1 | −17.5 | 57.7 | 38 |
| PHE | CG | A | 414 | −7.9 | −18.3 | 57.9 | 40 |
| PHE | CD1 | A | 414 | −7.3 | −18.9 | 56.8 | 39 |
| PHE | CD2 | A | 414 | −7.3 | −18.4 | 59.1 | 40 |
| PHE | CE1 | A | 414 | −6.1 | −19.7 | 56.9 | 40 |
| PHE | CE2 | A | 414 | −6.2 | −19.2 | 59.3 | 40 |
| PHE | CZ | A | 414 | −5.6 | −19.8 | 58.2 | 40 |
| PHE | C | A | 414 | −11.6 | −17.6 | 57.6 | 46 |
| PHE | O | A | 414 | −12.0 | −17.3 | 56.4 | 48 |
| VAL | N | A | 415 | −12.3 | −17.2 | 58.6 | 49 |
| VAL | CA | A | 415 | −13.5 | −16.3 | 58.5 | 56 |
| VAL | CB | A | 415 | −14.6 | −16.6 | 59.5 | 57 |
| VAL | CG1 | A | 415 | −15.8 | −15.8 | 59.3 | 54 |
| VAL | CG2 | A | 415 | −15.0 | −18.1 | 59.5 | 56 |
| VAL | C | A | 415 | −13.2 | −14.8 | 58.4 | 62 |
| VAL | O | A | 415 | −12.9 | −14.2 | 59.4 | 62 |
| THR | N | A | 416 | −13.4 | −14.3 | 57.2 | 69 |
| THR | CA | A | 416 | −13.2 | −12.8 | 57.0 | 76 |
| THR | CB | A | 416 | −11.9 | −12.6 | 56.1 | 78 |
| THR | OG1 | A | 416 | −10.8 | −13.1 | 56.7 | 81 |
| THR | CG2 | A | 416 | −11.7 | −11.1 | 55.9 | 79 |
| THR | C | A | 416 | −14.4 | −12.2 | 56.5 | 79 |
| THR | O | A | 416 | −15.0 | −12.6 | 55.5 | 80 |
| LEU | N | A | 417 | −14.8 | −11.0 | 57.1 | 83 |
| LEU | CA | A | 417 | −16.0 | −10.3 | 56.7 | 86 |
| LEU | CB | A | 417 | −16.8 | −9.9 | 57.9 | 87 |
| LEU | CG | A | 417 | −17.3 | −11.0 | 58.8 | 88 |
| LEU | CD1 | A | 417 | −18.0 | −10.5 | 60.0 | 88 |
| LEU | CD2 | A | 417 | −18.2 | −12.0 | 58.0 | 87 |
| LEU | C | A | 417 | −15.7 | −9.1 | 55.8 | 87 |
| LEU | O | A | 417 | −14.7 | −8.4 | 56.0 | 87 |
| ASP | N | A | 418 | −16.6 | −8.8 | 54.9 | 88 |
| ASP | CA | A | 418 | −16.5 | −7.7 | 54.0 | 88 |
| ASP | CB | A | 418 | −16.3 | −6.4 | 54.7 | 90 |
| ASP | CG | A | 418 | −17.5 | −6.1 | 55.7 | 92 |
| ASP | OD1 | A | 418 | −17.2 | −6.0 | 56.9 | 93 |
| ASP | OD2 | A | 418 | −18.6 | −6.0 | 55.2 | 92 |
| ASP | C | A | 418 | −15.4 | −7.8 | 52.8 | 86 |
| ASP | O | A | 418 | −15.1 | −6.8 | 52.2 | 87 |
| MET | N | A | 419 | −14.9 | −9.0 | 52.6 | 83 |
| MET | CA | A | 419 | −13.9 | −9.3 | 51.6 | 81 |
| MET | CB | A | 419 | −13.6 | −10.8 | 51.4 | 78 |
| MET | CG | A | 419 | −13.1 | −11.5 | 52.7 | 74 |
| MET | SD | A | 419 | −12.9 | −13.2 | 52.3 | 73 |
| MET | CE | A | 419 | −14.6 | −13.8 | 52.4 | 73 |
| MET | C | A | 419 | −14.3 | −8.7 | 50.3 | 83 |
| MET | O | A | 419 | −13.4 | −8.5 | 49.4 | 83 |
| GLU | N | A | 420 | −15.5 | −8.3 | 50.1 | 84 |
| GLU | CA | A | 420 | −16.0 | −7.7 | 48.8 | 86 |
| GLU | CB | A | 420 | −17.5 | −7.9 | 48.6 | 90 |
| GLU | CG | A | 420 | −18.0 | −7.3 | 47.3 | 93 |
| GLU | CD | A | 420 | −17.4 | −8.0 | 46.1 | 96 |
| GLU | OE1 | A | 420 | −16.6 | −8.9 | 46.3 | 97 |
| GLU | OE2 | A | 420 | −17.6 | −7.6 | 45.0 | 97 |
| GLU | C | A | 420 | −15.6 | −6.3 | 48.7 | 85 |
| GLU | O | A | 420 | −15.6 | −5.7 | 47.6 | 89 |
| ASP | N | A | 421 | −15.2 | −5.7 | 49.8 | 81 |
| ASP | CA | A | 421 | −14.8 | −4.3 | 49.8 | 75 |
| ASP | CB | A | 421 | −15.1 | −3.6 | 51.2 | 79 |
| ASP | CG | A | 421 | −16.5 | −3.6 | 51.5 | 83 |
| ASP | OD1 | A | 421 | −17.1 | −2.5 | 51.7 | 85 |
| ASP | OD2 | A | 421 | −17.1 | −4.7 | 51.6 | 85 |
| ASP | C | A | 421 | −13.3 | −4.2 | 49.5 | 69 |
| ASP | O | A | 421 | −12.7 | −3.2 | 49.5 | 67 |
| CYS | N | A | 422 | −12.7 | −5.4 | 49.3 | 62 |
| CYS | CA | A | 422 | −11.3 | −5.4 | 49.0 | 56 |
| CYS | C | A | 422 | −11.0 | −5.2 | 47.5 | 53 |
| CYS | O | A | 422 | −9.8 | −5.0 | 47.1 | 52 |
| CYS | CB | A | 422 | −10.7 | −6.8 | 49.4 | 53 |
| CYS | SG | A | 422 | −10.9 | −7.1 | 51.2 | 50 |
| GLY | N | A | 423 | −12.0 | −5.2 | 46.7 | 52 |
| GLY | CA | A | 423 | −11.8 | −5.0 | 45.3 | 51 |
| GLY | C | A | 423 | −11.8 | −3.6 | 44.9 | 54 |
| GLY | O | A | 423 | −12.4 | −2.7 | 45.6 | 56 |
| TYR | N | A | 424 | −11.1 | −3.2 | 43.9 | 56 |
| TYR | CA | A | 424 | −10.9 | −1.8 | 43.4 | 57 |
| TYR | CB | A | 424 | −9.5 | −1.5 | 43.1 | 59 |
| TYR | CG | A | 424 | −9.3 | 0.0 | 42.7 | 61 |
| TYR | CD1 | A | 424 | −9.5 | 1.0 | 43.6 | 61 |
| TYR | CE1 | A | 424 | −9.4 | 2.4 | 43.1 | 64 |
| TYR | CD2 | A | 424 | −9.1 | 0.3 | 41.3 | 61 |
| TYR | CE2 | A | 424 | −9.0 | 1.6 | 40.9 | 62 |
| TYR | CZ | A | 424 | −9.1 | 2.6 | 41.8 | 63 |
| TYR | OH | A | 424 | −9.1 | 3.9 | 41.4 | 68 |
| TYR | C | A | 424 | −11.8 | −1.6 | 42.2 | 57 |
| TYR | O | A | 424 | −11.8 | −2.4 | 41.3 | 59 |
| ASN | N | A | 425 | −12.6 | −0.5 | 42.1 | 58 |
| ASN | CA | A | 425 | −13.4 | −0.2 | 41.0 | 58 |
| ASN | CB | A | 425 | −14.9 | −0.3 | 41.4 | 58 |
| ASN | CG | A | 425 | −15.3 | −1.6 | 41.9 | 58 |
| ASN | OD1 | A | 425 | −16.1 | −2.3 | 41.3 | 59 |
| ASN | ND2 | A | 425 | −14.8 | −2.0 | 43.1 | 60 |
| ASN | C | A | 425 | −13.1 | 1.1 | 40.3 | 56 |
| ASN | O | A | 425 | −12.6 | 2.1 | 41.0 | 53 |
| SER | N | B | 38 | 39.6 | 7.4 | 12.8 | 72 |
| SER | CA | B | 38 | 39.5 | 6.1 | 12.0 | 73 |
| SER | CB | B | 38 | 38.4 | 5.2 | 12.6 | 77 |
| SER | OG | B | 38 | 38.7 | 4.8 | 14.0 | 80 |
| SER | C | B | 38 | 39.2 | 6.4 | 10.6 | 69 |
| SER | O | B | 38 | 39.1 | 5.5 | 9.7 | 69 |
| PHE | N | B | 39 | 39.0 | 7.7 | 10.3 | 63 |
| PHE | CA | B | 39 | 38.7 | 8.2 | 8.9 | 55 |
| PHE | CB | B | 39 | 37.2 | 8.5 | 8.7 | 49 |
| PHE | CG | B | 39 | 36.3 | 7.4 | 9.0 | 46 |
| PHE | CD1 | B | 39 | 35.9 | 7.0 | 10.3 | 44 |
| PHE | CD2 | B | 39 | 35.8 | 6.6 | 7.9 | 46 |
| PHE | CE1 | B | 39 | 35.1 | 6.0 | 10.5 | 45 |
| PHE | CE2 | B | 39 | 34.9 | 5.6 | 8.1 | 45 |
| PHE | CZ | B | 39 | 34.6 | 5.2 | 9.4 | 45 |
| PHE | C | B | 39 | 39.6 | 9.4 | 8.5 | 54 |
| PHE | O | B | 39 | 39.2 | 10.2 | 7.7 | 54 |
| VAL | N | B | 40 | 40.8 | 9.4 | 9.1 | 53 |
| VAL | CA | B | 40 | 41.7 | 10.5 | 8.9 | 54 |
| VAL | CB | B | 40 | 43.1 | 10.2 | 9.5 | 58 |
| VAL | CG1 | B | 40 | 44.1 | 11.3 | 9.2 | 59 |
| VAL | CG2 | B | 40 | 43.0 | 9.8 | 10.9 | 61 |
| VAL | C | B | 40 | 41.9 | 11.0 | 7.4 | 52 |
| VAL | O | B | 40 | 42.2 | 12.1 | 7.2 | 51 |
| GLU | N | B | 41 | 41.7 | 10.1 | 6.5 | 49 |
| GLU | CA | B | 41 | 41.9 | 10.4 | 5.0 | 47 |
| GLU | CB | B | 41 | 42.0 | 9.2 | 4.2 | 53 |
| GLU | CG | B | 41 | 42.1 | 9.5 | 2.7 | 64 |
| GLU | CD | B | 41 | 42.3 | 8.2 | 1.9 | 70 |
| GLU | OE1 | B | 41 | 42.2 | 7.1 | 2.5 | 72 |
| GLU | OE2 | B | 41 | 42.4 | 8.2 | 0.7 | 71 |
| GLU | C | B | 41 | 40.8 | 11.4 | 4.5 | 42 |
| GLU | O | B | 41 | 41.0 | 12.0 | 3.5 | 38 |
| MET | N | B | 42 | 39.6 | 11.4 | 5.2 | 34 |
| MET | CA | B | 42 | 38.5 | 12.2 | 4.7 | 30 |
| MET | CB | B | 42 | 37.2 | 11.5 | 4.7 | 30 |
| MET | CG | B | 42 | 37.2 | 10.3 | 3.8 | 30 |
| MET | SD | B | 42 | 35.6 | 9.5 | 3.9 | 31 |
| MET | CE | B | 42 | 36.0 | 8.1 | 4.8 | 26 |
| MET | C | B | 42 | 38.4 | 13.5 | 5.6 | 28 |
| MET | O | B | 42 | 37.6 | 14.4 | 5.3 | 27 |
| VAL | N | B | 43 | 39.2 | 13.5 | 6.7 | 27 |
| VAL | CA | B | 43 | 39.2 | 14.7 | 7.5 | 26 |
| VAL | CB | B | 43 | 40.0 | 14.5 | 8.8 | 26 |
| VAL | CG1 | B | 43 | 40.0 | 15.8 | 9.6 | 27 |
| VAL | CG2 | B | 43 | 39.4 | 13.4 | 9.7 | 24 |
| VAL | C | B | 43 | 39.7 | 15.9 | 6.8 | 29 |
| VAL | O | B | 43 | 40.7 | 15.9 | 6.1 | 26 |
| ASP | N | B | 44 | 38.9 | 17.0 | 6.8 | 33 |
| ASP | CA | B | 44 | 39.3 | 18.2 | 6.2 | 31 |
| ASP | CB | B | 44 | 40.7 | 18.6 | 6.6 | 35 |
| ASP | CG | B | 44 | 41.1 | 20.0 | 6.1 | 42 |
| ASP | OD1 | B | 44 | 40.2 | 20.8 | 5.9 | 42 |
| ASP | OD2 | B | 44 | 42.3 | 20.3 | 6.0 | 44 |
| ASP | C | B | 44 | 39.2 | 18.2 | 4.7 | 30 |
| ASP | O | B | 44 | 39.8 | 18.9 | 3.9 | 25 |
| ASN | N | B | 45 | 38.3 | 17.3 | 4.1 | 29 |
| ASN | CA | B | 45 | 38.1 | 17.1 | 2.7 | 27 |
| ASN | CB | B | 45 | 37.6 | 15.7 | 2.3 | 24 |
| ASN | CG | B | 45 | 36.3 | 15.4 | 2.8 | 19 |
| ASN | OD1 | B | 45 | 35.7 | 16.1 | 3.6 | 18 |
| ASN | ND2 | B | 45 | 35.8 | 14.2 | 2.5 | 17 |
| ASN | C | B | 45 | 37.2 | 18.2 | 2.1 | 25 |
| ASN | O | B | 45 | 36.8 | 18.2 | 1.0 | 24 |
| LEU | N | B | 46 | 36.7 | 19.1 | 3.0 | 23 |
| LEU | CA | B | 46 | 35.8 | 20.2 | 2.6 | 27 |
| LEU | CB | B | 46 | 34.6 | 20.2 | 3.4 | 26 |
| LEU | CG | B | 46 | 33.7 | 19.0 | 3.4 | 26 |
| LEU | CD1 | B | 46 | 32.5 | 19.1 | 4.3 | 24 |
| LEU | CD2 | B | 46 | 33.3 | 18.6 | 1.9 | 24 |
| LEU | C | B | 46 | 36.4 | 21.6 | 2.5 | 27 |
| LEU | O | B | 46 | 37.2 | 22.0 | 3.4 | 27 |
| ARG | N | B | 47 | 36.1 | 22.3 | 1.5 | 30 |
| ARG | CA | B | 47 | 36.5 | 23.7 | 1.2 | 26 |
| ARG | CB | B | 47 | 37.6 | 23.9 | 0.2 | 30 |
| ARG | CG | B | 47 | 38.9 | 23.3 | 0.6 | 34 |
| ARG | CD | B | 47 | 39.5 | 24.0 | 1.8 | 44 |
| ARG | NE | B | 47 | 40.8 | 23.5 | 2.3 | 46 |
| ARG | CZ | B | 47 | 40.9 | 22.8 | 3.4 | 47 |
| ARG | NH1 | B | 47 | 39.9 | 22.6 | 4.2 | 46 |
| ARG | NH2 | B | 47 | 42.1 | 22.4 | 3.8 | 47 |
| ARG | C | B | 47 | 35.3 | 24.5 | 0.8 | 27 |
| ARG | O | B | 47 | 34.2 | 24.0 | 0.5 | 25 |
| GLY | N | B | 48 | 35.4 | 25.9 | 0.9 | 29 |
| GLY | CA | B | 48 | 34.3 | 26.7 | 0.5 | 34 |
| GLY | C | B | 48 | 34.5 | 28.1 | 0.8 | 38 |
| GLY | O | B | 48 | 35.5 | 28.5 | 1.5 | 39 |
| LYS | N | B | 49 | 33.7 | 29.0 | 0.2 | 37 |
| LYS | CA | B | 49 | 33.8 | 30.4 | 0.5 | 36 |
| LYS | CB | B | 49 | 33.9 | 31.2 | −0.8 | 39 |
| LYS | CG | B | 49 | 34.0 | 32.7 | −0.6 | 46 |
| LYS | CD | B | 49 | 34.1 | 33.5 | −1.8 | 51 |
| LYS | CE | B | 49 | 34.3 | 35.0 | −1.6 | 55 |
| LYS | NZ | B | 49 | 33.1 | 35.6 | −0.8 | 54 |
| LYS | C | B | 49 | 32.5 | 30.8 | 1.3 | 33 |
| LYS | O | B | 49 | 31.4 | 30.4 | 1.0 | 35 |
| SER | N | B | 50 | 32.8 | 31.6 | 2.4 | 30 |
| SER | CA | B | 50 | 31.7 | 32.0 | 3.2 | 32 |
| SER | CB | B | 50 | 32.1 | 33.2 | 4.1 | 30 |
| SER | OG | B | 50 | 31.1 | 33.6 | 5.0 | 35 |
| SER | C | B | 50 | 30.4 | 32.4 | 2.5 | 29 |
| SER | O | B | 50 | 30.4 | 33.3 | 1.6 | 29 |
| GLY | N | B | 51 | 29.3 | 31.8 | 2.8 | 25 |
| GLY | CA | B | 51 | 28.0 | 32.1 | 2.2 | 23 |
| GLY | C | B | 51 | 27.9 | 31.5 | 0.8 | 24 |
| GLY | O | B | 51 | 26.9 | 31.8 | 0.1 | 20 |
| GLN | N | B | 52 | 28.8 | 30.6 | 0.3 | 22 |
| GLN | CA | B | 52 | 28.7 | 30.1 | −1.0 | 21 |
| GLN | CB | B | 52 | 29.8 | 30.7 | −1.9 | 21 |
| GLN | CG | B | 52 | 29.6 | 32.2 | −2.0 | 26 |
| GLN | CD | B | 52 | 30.7 | 32.8 | −2.9 | 29 |
| GLN | OE1 | B | 52 | 31.5 | 32.1 | −3.5 | 30 |
| GLN | NE2 | B | 52 | 30.6 | 34.1 | −3.1 | 32 |
| GLN | C | B | 52 | 28.7 | 28.5 | −1.0 | 23 |
| GLN | O | B | 52 | 29.0 | 27.9 | −2.0 | 21 |
| GLY | N | B | 53 | 28.4 | 28.0 | 0.2 | 19 |
| GLY | CA | B | 53 | 28.4 | 26.5 | 0.4 | 20 |
| GLY | C | B | 53 | 29.7 | 25.8 | 0.6 | 24 |
| GLY | O | B | 53 | 30.8 | 26.5 | 0.7 | 29 |
| TYR | N | B | 54 | 29.6 | 24.5 | 0.7 | 24 |
| TYR | CA | B | 54 | 30.8 | 23.7 | 0.9 | 23 |
| TYR | CB | B | 54 | 30.8 | 23.0 | 2.3 | 21 |
| TYR | CG | B | 54 | 30.8 | 23.9 | 3.5 | 21 |
| TYR | CD1 | B | 54 | 29.7 | 24.6 | 3.9 | 19 |
| TYR | CE1 | B | 54 | 29.7 | 25.4 | 5.0 | 24 |
| TYR | CD2 | B | 54 | 32.0 | 24.1 | 4.2 | 25 |
| TYR | CE2 | B | 54 | 32.1 | 24.9 | 5.3 | 24 |
| TYR | CZ | B | 54 | 30.9 | 25.6 | 5.7 | 25 |
| TYR | OH | B | 54 | 31.1 | 26.4 | 6.8 | 20 |
| TYR | C | B | 54 | 30.9 | 22.6 | −0.2 | 23 |
| TYR | O | B | 54 | 29.9 | 22.1 | −0.6 | 24 |
| TYR | N | B | 55 | 32.2 | 22.4 | −0.7 | 23 |
| TYR | CA | B | 55 | 32.4 | 21.5 | −1.7 | 23 |
| TYR | CB | B | 55 | 32.7 | 22.2 | −3.1 | 22 |
| TYR | CG | B | 55 | 33.9 | 23.1 | −3.0 | 23 |
| TYR | CD1 | B | 55 | 35.2 | 22.6 | −3.3 | 20 |
| TYR | CE1 | B | 55 | 36.3 | 23.4 | −3.3 | 21 |
| TYR | CD2 | B | 55 | 33.8 | 24.5 | −2.8 | 24 |
| TYR | CE2 | B | 55 | 34.9 | 25.3 | −2.8 | 24 |
| TYR | CZ | B | 55 | 36.2 | 24.8 | −3.0 | 24 |
| TYR | OH | B | 55 | 37.3 | 25.6 | −3.0 | 23 |
| TYR | C | B | 55 | 33.5 | 20.5 | −1.4 | 28 |
| TYR | O | B | 55 | 34.4 | 20.7 | −0.6 | 20 |
| VAL | N | B | 56 | 33.4 | 19.3 | −2.1 | 23 |
| VAL | CA | B | 56 | 34.3 | 18.2 | −2.0 | 21 |
| VAL | CB | B | 56 | 33.6 | 17.0 | −1.4 | 21 |
| VAL | CG1 | B | 56 | 32.6 | 16.4 | −2.4 | 17 |
| VAL | CG2 | B | 56 | 34.7 | 15.9 | −1.1 | 16 |
| VAL | C | B | 56 | 34.9 | 18.0 | −3.4 | 24 |
| VAL | O | B | 56 | 34.2 | 18.2 | −4.4 | 22 |
| GLU | N | B | 57 | 36.1 | 17.5 | −3.4 | 24 |
| GLU | CA | B | 57 | 36.7 | 17.2 | −4.7 | 24 |
| GLU | CB | B | 57 | 38.2 | 17.2 | −4.7 | 24 |
| GLU | CG | B | 57 | 38.8 | 17.0 | −6.1 | 24 |
| GLU | CD | B | 57 | 40.3 | 17.0 | −6.1 | 30 |
| GLU | OE1 | B | 57 | 40.9 | 18.1 | −6.2 | 34 |
| GLU | OE2 | B | 57 | 40.9 | 16.0 | −5.9 | 33 |
| GLU | C | B | 57 | 36.2 | 15.9 | −5.3 | 25 |
| GLU | O | B | 57 | 36.1 | 14.9 | −4.5 | 22 |
| MET | N | B | 58 | 35.8 | 15.8 | −6.5 | 21 |
| MET | CA | B | 58 | 35.3 | 14.6 | −7.2 | 22 |
| MET | CB | B | 58 | 33.8 | 14.5 | −7.2 | 22 |
| MET | CG | B | 58 | 33.1 | 14.5 | −5.8 | 18 |
| MET | SD | B | 58 | 31.3 | 14.4 | −6.0 | 19 |
| MET | CE | B | 58 | 31.1 | 12.7 | −6.3 | 19 |
| MET | C | B | 58 | 35.8 | 14.5 | −8.6 | 26 |
| MET | O | B | 58 | 36.3 | 15.4 | −9.2 | 26 |
| THR | N | B | 59 | 35.8 | 13.2 | −9.1 | 26 |
| THR | CA | B | 59 | 36.3 | 13.0 | −10.5 | 24 |
| THR | CB | B | 59 | 37.6 | 12.2 | −10.5 | 25 |
| THR | OG1 | B | 59 | 37.4 | 10.9 | −9.9 | 29 |
| THR | CG2 | B | 59 | 38.7 | 12.9 | −9.9 | 23 |
| THR | C | B | 59 | 35.1 | 12.3 | −11.2 | 28 |
| THR | O | B | 59 | 34.4 | 11.5 | −10.6 | 28 |
| VAL | N | B | 60 | 35.0 | 12.6 | −12.5 | 24 |
| VAL | CA | B | 60 | 34.0 | 12.0 | −13.3 | 23 |
| VAL | CB | B | 60 | 32.8 | 12.9 | −13.6 | 25 |
| VAL | CG1 | B | 60 | 32.1 | 13.3 | −12.2 | 24 |
| VAL | CG2 | B | 60 | 33.2 | 14.1 | −14.3 | 27 |
| VAL | C | B | 60 | 34.6 | 11.6 | −14.6 | 25 |
| VAL | O | B | 60 | 35.4 | 12.3 | −15.2 | 28 |
| GLY | N | B | 61 | 34.2 | 10.4 | −15.1 | 23 |
| GLY | CA | B | 61 | 34.7 | 9.9 | −16.4 | 22 |
| GLY | C | B | 61 | 36.0 | 9.1 | −16.4 | 28 |
| GLY | O | B | 61 | 36.7 | 9.0 | −15.4 | 29 |
| SER | N | B | 62 | 36.3 | 8.7 | −17.7 | 28 |
| SER | CA | B | 62 | 37.5 | 7.9 | −17.9 | 33 |
| SER | CB | B | 62 | 37.2 | 6.4 | −18.1 | 30 |
| SER | OG | B | 62 | 36.6 | 5.9 | −16.9 | 33 |
| SER | C | B | 62 | 38.2 | 8.5 | −19.2 | 33 |
| SER | O | B | 62 | 37.6 | 8.3 | −20.3 | 35 |
| PRO | N | B | 63 | 39.4 | 9.1 | −19.1 | 29 |
| PRO | CD | B | 63 | 40.0 | 9.8 | −20.2 | 27 |
| PRO | CA | B | 63 | 40.1 | 9.3 | −17.9 | 29 |
| PRO | CB | B | 63 | 41.5 | 9.7 | −18.4 | 31 |
| PRO | CG | B | 63 | 41.1 | 10.6 | −19.5 | 28 |
| PRO | C | B | 63 | 39.5 | 10.4 | −16.9 | 32 |
| PRO | O | B | 63 | 38.9 | 11.4 | −17.4 | 32 |
| PRO | N | B | 64 | 39.6 | 10.2 | −15.6 | 29 |
| PRO | CD | B | 64 | 40.2 | 9.0 | −15.0 | 34 |
| PRO | CA | B | 64 | 39.2 | 11.0 | −14.5 | 29 |
| PRO | CB | B | 64 | 40.0 | 10.6 | −13.4 | 33 |
| PRO | CG | B | 64 | 39.9 | 9.1 | −13.5 | 37 |
| PRO | C | B | 64 | 39.2 | 12.6 | −14.7 | 26 |
| PRO | O | B | 64 | 40.3 | 13.1 | −14.9 | 22 |
| GLN | N | B | 65 | 38.0 | 13.2 | −14.7 | 26 |
| GLN | CA | B | 65 | 38.0 | 14.7 | −14.8 | 23 |
| GLN | CB | B | 65 | 36.9 | 15.1 | −15.9 | 20 |
| GLN | CG | B | 65 | 37.1 | 14.5 | −17.3 | 22 |
| GLN | CD | B | 65 | 36.0 | 15.0 | −18.2 | 26 |
| GLN | OE1 | B | 65 | 35.3 | 16.0 | −17.9 | 27 |
| GLN | NE2 | B | 65 | 35.8 | 14.3 | −19.3 | 27 |
| GLN | C | B | 65 | 37.7 | 15.2 | −13.4 | 26 |
| GLN | O | B | 65 | 36.7 | 14.9 | −12.8 | 22 |
| THR | N | B | 66 | 38.5 | 16.1 | −13.0 | 25 |
| THR | CA | B | 66 | 38.4 | 16.7 | −11.7 | 25 |
| THR | CB | B | 66 | 39.8 | 17.1 | −11.1 | 24 |
| THR | OG1 | B | 66 | 40.7 | 16.0 | −11.1 | 29 |
| THR | CG2 | B | 66 | 39.6 | 17.7 | −9.7 | 24 |
| THR | C | B | 66 | 37.4 | 17.9 | −11.6 | 26 |
| THR | O | B | 66 | 37.4 | 18.8 | −12.4 | 23 |
| LEU | N | B | 67 | 36.6 | 17.8 | −10.6 | 26 |
| LEU | CA | B | 67 | 35.5 | 18.8 | −10.4 | 21 |
| LEU | CB | B | 67 | 34.2 | 18.4 | −11.1 | 21 |
| LEU | CG | B | 67 | 34.4 | 18.3 | −12.6 | 21 |
| LEU | CD1 | B | 67 | 33.2 | 17.7 | −13.3 | 25 |
| LEU | CD2 | B | 67 | 34.8 | 19.7 | −13.3 | 22 |
| LEU | C | B | 67 | 35.2 | 19.0 | −8.9 | 22 |
| LEU | O | B | 67 | 35.2 | 18.1 | −8.1 | 24 |
| ASN | N | B | 68 | 35.0 | 20.3 | −8.5 | 19 |
| ASN | CA | B | 68 | 34.6 | 20.6 | −7.1 | 20 |
| ASN | CB | B | 68 | 35.1 | 21.9 | −6.6 | 19 |
| ASN | CG | B | 68 | 36.6 | 21.9 | −6.4 | 21 |
| ASN | OD1 | B | 68 | 37.2 | 22.9 | −6.7 | 27 |
| ASN | ND2 | B | 68 | 37.2 | 20.8 | −6.0 | 14 |
| ASN | C | B | 68 | 33.0 | 20.5 | −7.1 | 23 |
| ASN | O | B | 68 | 32.4 | 21.1 | −7.9 | 23 |
| ILE | N | B | 69 | 32.5 | 19.7 | −6.1 | 21 |
| ILE | CA | B | 69 | 31.1 | 19.5 | −6.0 | 21 |
| ILE | CB | B | 69 | 30.7 | 18.0 | −6.3 | 23 |
| ILE | CG2 | B | 69 | 29.2 | 17.9 | −6.3 | 23 |
| ILE | CG1 | B | 69 | 31.3 | 17.5 | −7.6 | 22 |
| ILE | CD1 | B | 69 | 30.8 | 18.2 | −8.9 | 29 |
| ILE | C | B | 69 | 30.6 | 19.9 | −4.7 | 22 |
| ILE | O | B | 69 | 31.0 | 19.5 | −3.6 | 19 |
| LEU | N | B | 70 | 29.6 | 20.8 | −4.7 | 20 |
| LEU | CA | B | 70 | 28.9 | 21.3 | −3.6 | 20 |
| LEU | CB | B | 70 | 28.1 | 22.6 | −3.9 | 20 |
| LEU | CG | B | 70 | 27.4 | 23.2 | −2.7 | 24 |
| LEU | CD1 | B | 70 | 26.8 | 24.6 | −3.1 | 24 |
| LEU | CD2 | B | 70 | 26.2 | 22.4 | −2.2 | 26 |
| LEU | C | B | 70 | 28.1 | 20.3 | −2.9 | 22 |
| LEU | O | B | 70 | 27.2 | 19.7 | −3.5 | 29 |
| VAL | N | B | 71 | 28.3 | 20.0 | −1.6 | 26 |
| VAL | CA | B | 71 | 27.6 | 19.0 | −0.9 | 26 |
| VAL | CB | B | 71 | 28.4 | 18.5 | 0.3 | 27 |
| VAL | CG1 | B | 71 | 27.6 | 17.5 | 1.1 | 33 |
| VAL | CG2 | B | 71 | 29.7 | 17.8 | −0.2 | 26 |
| VAL | C | B | 71 | 26.3 | 19.6 | −0.4 | 29 |
| VAL | O | B | 71 | 26.2 | 20.5 | 0.4 | 26 |
| ASP | N | B | 72 | 25.2 | 19.0 | −0.9 | 28 |
| ASP | CA | B | 72 | 23.8 | 19.5 | −0.6 | 26 |
| ASP | CB | B | 72 | 23.2 | 20.1 | −1.8 | 27 |
| ASP | CG | B | 72 | 21.9 | 20.7 | −1.6 | 28 |
| ASP | OD1 | B | 72 | 21.5 | 21.0 | −0.5 | 33 |
| ASP | OD2 | B | 72 | 21.1 | 20.9 | −2.6 | 24 |
| ASP | C | B | 72 | 22.9 | 18.4 | 0.0 | 27 |
| ASP | O | B | 72 | 22.4 | 17.6 | −0.8 | 25 |
| THR | N | B | 73 | 22.6 | 18.5 | 1.3 | 25 |
| THR | CA | B | 73 | 21.6 | 17.5 | 1.8 | 22 |
| THR | CB | B | 73 | 21.9 | 17.3 | 3.4 | 23 |
| THR | OG1 | B | 73 | 21.7 | 18.5 | 4.1 | 23 |
| THR | CG2 | B | 73 | 23.2 | 16.7 | 3.7 | 18 |
| THR | C | B | 73 | 20.2 | 17.9 | 1.6 | 29 |
| THR | O | B | 73 | 19.3 | 17.2 | 1.9 | 27 |
| GLY | N | B | 74 | 20.0 | 19.0 | 0.9 | 27 |
| GLY | CA | B | 74 | 18.7 | 19.5 | 0.6 | 28 |
| GLY | C | B | 74 | 18.1 | 19.1 | −0.8 | 27 |
| GLY | O | B | 74 | 17.0 | 19.5 | −1.1 | 23 |
| SER | N | B | 75 | 18.9 | 18.3 | −1.5 | 26 |
| SER | CA | B | 75 | 18.4 | 17.8 | −2.9 | 22 |
| SER | CB | B | 75 | 18.9 | 18.7 | −4.0 | 23 |
| SER | OG | B | 75 | 20.3 | 18.8 | −4.1 | 28 |
| SER | C | B | 75 | 18.9 | 16.4 | −3.1 | 22 |
| SER | O | B | 75 | 19.7 | 15.9 | −2.3 | 24 |
| SER | N | B | 76 | 18.4 | 15.8 | −4.2 | 20 |
| SER | CA | B | 76 | 18.8 | 14.4 | −4.4 | 23 |
| SER | CB | B | 76 | 17.6 | 13.5 | −4.2 | 22 |
| SER | OG | B | 76 | 17.2 | 13.7 | −2.9 | 28 |
| SER | C | B | 76 | 19.4 | 14.2 | −5.8 | 26 |
| SER | O | B | 76 | 19.6 | 13.0 | −6.2 | 24 |
| ASN | N | B | 77 | 19.8 | 15.2 | −6.5 | 26 |
| ASN | CA | B | 77 | 20.4 | 15.1 | −7.8 | 23 |
| ASN | CB | B | 77 | 19.7 | 15.9 | −8.9 | 23 |
| ASN | CG | B | 77 | 18.3 | 15.4 | −9.2 | 24 |
| ASN | OD1 | B | 77 | 17.3 | 15.7 | −8.5 | 24 |
| ASN | ND2 | B | 77 | 18.2 | 14.6 | −10.2 | 18 |
| ASN | C | B | 77 | 21.9 | 15.4 | −7.8 | 23 |
| ASN | O | B | 77 | 22.4 | 16.3 | −7.2 | 25 |
| PHE | N | B | 78 | 22.6 | 14.6 | −8.6 | 21 |
| PHE | CA | B | 78 | 24.1 | 14.8 | −8.8 | 22 |
| PHE | CB | B | 78 | 24.8 | 13.5 | −8.8 | 23 |
| PHE | CG | B | 78 | 26.3 | 13.6 | −9.0 | 22 |
| PHE | CD1 | B | 78 | 27.0 | 12.7 | −9.6 | 24 |
| PHE | CD2 | B | 78 | 27.0 | 14.8 | −8.6 | 23 |
| PHE | CE1 | B | 78 | 28.4 | 12.8 | −9.9 | 23 |
| PHE | CE2 | B | 78 | 28.3 | 15.0 | −8.8 | 22 |
| PHE | CZ | B | 78 | 29.1 | 14.0 | −9.4 | 20 |
| PHE | C | B | 78 | 24.1 | 15.5 | −10.1 | 22 |
| PHE | O | B | 78 | 23.7 | 15.0 | −11.1 | 22 |
| ALA | N | B | 79 | 24.7 | 16.7 | −10.1 | 24 |
| ALA | CA | B | 79 | 24.8 | 17.5 | −11.4 | 22 |
| ALA | CB | B | 79 | 23.6 | 18.5 | −11.5 | 23 |
| ALA | C | B | 79 | 26.1 | 18.2 | −11.4 | 23 |
| ALA | O | B | 79 | 26.6 | 18.7 | −10.4 | 22 |
| VAL | N | B | 80 | 26.6 | 18.4 | −12.7 | 24 |
| VAL | CA | B | 80 | 27.9 | 19.0 | −12.9 | 24 |
| VAL | CB | B | 80 | 29.0 | 18.1 | −13.1 | 25 |
| VAL | CG1 | B | 80 | 29.2 | 17.2 | −11.9 | 26 |
| VAL | CG2 | B | 80 | 28.8 | 17.2 | −14.4 | 19 |
| VAL | C | B | 80 | 27.8 | 20.0 | −14.2 | 25 |
| VAL | O | B | 80 | 27.0 | 19.7 | −15.1 | 25 |
| GLY | N | B | 81 | 28.5 | 21.1 | −14.1 | 25 |
| GLY | CA | B | 81 | 28.5 | 21.9 | −15.3 | 19 |
| GLY | C | B | 81 | 29.0 | 21.1 | −16.4 | 25 |
| GLY | O | B | 81 | 30.0 | 20.4 | −16.3 | 19 |
| ALA | N | B | 82 | 28.3 | 21.1 | −17.6 | 20 |
| ALA | CA | B | 82 | 28.7 | 20.3 | −18.8 | 23 |
| ALA | CB | B | 82 | 27.7 | 19.2 | −19.0 | 23 |
| ALA | C | B | 82 | 28.8 | 21.2 | −20.0 | 26 |
| ALA | O | B | 82 | 28.9 | 20.6 | −21.1 | 24 |
| ALA | N | B | 83 | 28.8 | 22.5 | −19.9 | 25 |
| ALA | CA | B | 83 | 28.9 | 23.4 | −21.0 | 28 |
| ALA | CB | B | 83 | 27.5 | 23.6 | −21.6 | 27 |
| ALA | C | B | 83 | 29.5 | 24.7 | −20.4 | 33 |
| ALA | O | B | 83 | 29.3 | 25.0 | −19.2 | 32 |
| PRO | N | B | 84 | 30.1 | 25.5 | −21.3 | 34 |
| PRO | CD | B | 84 | 30.6 | 25.1 | −22.7 | 35 |
| PRO | CA | B | 84 | 30.7 | 26.8 | −21.0 | 31 |
| PRO | CB | B | 84 | 31.0 | 27.4 | −22.3 | 36 |
| PRO | CG | B | 84 | 31.7 | 26.2 | −23.0 | 37 |
| PRO | C | B | 84 | 29.8 | 27.7 | −20.2 | 29 |
| PRO | O | B | 84 | 28.6 | 27.7 | −20.4 | 29 |
| HIS | N | B | 85 | 30.3 | 28.4 | −19.2 | 29 |
| HIS | CA | B | 85 | 29.5 | 29.3 | −18.4 | 29 |
| HIS | CB | B | 85 | 28.6 | 28.6 | −17.3 | 25 |
| HIS | CG | B | 85 | 27.8 | 29.5 | −16.5 | 25 |
| HIS | CD2 | B | 85 | 26.4 | 29.8 | −16.5 | 23 |
| HIS | ND1 | B | 85 | 28.3 | 30.4 | −15.6 | 25 |
| HIS | CE1 | B | 85 | 27.3 | 31.2 | −15.1 | 23 |
| HIS | NE2 | B | 85 | 26.2 | 30.8 | −15.6 | 24 |
| HIS | C | B | 85 | 30.4 | 30.3 | −17.7 | 30 |
| HIS | O | B | 85 | 31.5 | 30.0 | −17.1 | 34 |
| PRO | N | B | 86 | 30.1 | 31.6 | −17.7 | 32 |
| PRO | CD | B | 86 | 29.0 | 32.2 | −18.5 | 30 |
| PRO | CA | B | 86 | 30.9 | 32.7 | −17.2 | 29 |
| PRO | CB | B | 86 | 29.9 | 33.8 | −17.1 | 31 |
| PRO | CG | B | 86 | 29.3 | 33.7 | −18.5 | 33 |
| PRO | C | B | 86 | 31.4 | 32.4 | −15.8 | 27 |
| PRO | O | B | 86 | 32.5 | 32.8 | −15.4 | 27 |
| PHE | N | B | 87 | 30.7 | 31.6 | −15.0 | 27 |
| PHE | CA | B | 87 | 31.1 | 31.3 | −13.7 | 26 |
| PHE | CB | B | 87 | 29.9 | 31.5 | −12.7 | 30 |
| PHE | CG | B | 87 | 29.4 | 32.9 | −12.7 | 31 |
| PHE | CD1 | B | 87 | 28.2 | 33.2 | −12.1 | 30 |
| PHE | CD2 | B | 87 | 30.1 | 34.0 | −13.3 | 29 |
| PHE | CE1 | B | 87 | 27.7 | 34.6 | −12.0 | 33 |
| PHE | CE2 | B | 87 | 29.6 | 35.3 | −13.3 | 31 |
| PHE | CZ | B | 87 | 28.4 | 35.6 | −12.6 | 28 |
| PHE | C | B | 87 | 31.7 | 29.9 | −13.5 | 24 |
| PHE | O | B | 87 | 32.0 | 29.6 | −12.4 | 27 |
| LEU | N | B | 88 | 31.9 | 29.2 | −14.6 | 19 |
| LEU | CA | B | 88 | 32.6 | 27.9 | −14.5 | 22 |
| LEU | CB | B | 88 | 31.8 | 26.9 | −15.4 | 25 |
| LEU | CG | B | 88 | 30.4 | 26.5 | −14.9 | 21 |
| LEU | CD1 | B | 88 | 29.7 | 25.6 | −15.8 | 21 |
| LEU | CD2 | B | 88 | 30.5 | 25.9 | −13.5 | 25 |
| LEU | C | B | 88 | 34.0 | 27.9 | −14.9 | 25 |
| LEU | O | B | 88 | 34.4 | 28.3 | −16.0 | 29 |
| HIS | N | B | 89 | 34.9 | 27.6 | −13.9 | 20 |
| HIS | CA | B | 89 | 36.3 | 27.5 | −14.2 | 26 |
| HIS | CB | B | 89 | 37.1 | 27.2 | −12.9 | 26 |
| HIS | CG | B | 89 | 37.0 | 28.3 | −11.8 | 23 |
| HIS | CD2 | B | 89 | 36.9 | 28.1 | −10.5 | 19 |
| HIS | ND1 | B | 89 | 37.0 | 29.6 | −12.1 | 26 |
| HIS | CE1 | B | 89 | 37.0 | 30.3 | −10.9 | 22 |
| HIS | NE2 | B | 89 | 36.9 | 29.4 | −9.9 | 24 |
| HIS | C | B | 89 | 36.6 | 26.3 | −15.1 | 28 |
| HIS | O | B | 89 | 37.6 | 26.3 | −15.8 | 29 |
| ARG | N | B | 90 | 35.7 | 25.4 | −15.2 | 24 |
| ARG | CA | B | 90 | 35.9 | 24.2 | −15.9 | 22 |
| ARG | CB | B | 90 | 37.0 | 23.3 | −15.3 | 22 |
| ARG | CG | B | 90 | 36.8 | 23.0 | −13.8 | 23 |
| ARG | CD | B | 90 | 37.9 | 22.2 | −13.2 | 22 |
| ARG | NE | B | 90 | 37.6 | 22.0 | −11.8 | 21 |
| ARG | CZ | B | 90 | 38.5 | 21.6 | −10.9 | 21 |
| ARG | NH1 | B | 90 | 39.7 | 21.3 | −11.3 | 22 |
| ARG | NH2 | B | 90 | 38.2 | 21.5 | −9.6 | 21 |
| ARG | C | B | 90 | 34.6 | 23.4 | −15.9 | 24 |
| ARG | O | B | 90 | 33.7 | 23.6 | −15.0 | 22 |
| TYR | N | B | 91 | 34.4 | 22.4 | −16.8 | 27 |
| TYR | CA | B | 91 | 33.2 | 21.6 | −16.9 | 29 |
| TYR | CB | B | 91 | 32.1 | 22.4 | −17.7 | 31 |
| TYR | CG | B | 91 | 32.5 | 22.6 | −19.1 | 31 |
| TYR | CD1 | B | 91 | 32.3 | 21.6 | −20.1 | 31 |
| TYR | CE1 | B | 91 | 32.7 | 21.9 | −21.4 | 37 |
| TYR | CD2 | B | 91 | 33.0 | 23.9 | −19.5 | 32 |
| TYR | CE2 | B | 91 | 33.4 | 24.1 | −20.9 | 30 |
| TYR | CZ | B | 91 | 33.2 | 23.1 | −21.8 | 34 |
| TYR | OH | B | 91 | 33.5 | 23.3 | −23.1 | 35 |
| TYR | C | B | 91 | 33.4 | 20.2 | −17.5 | 28 |
| TYR | O | B | 91 | 34.4 | 19.9 | −18.2 | 25 |
| TYR | N | B | 92 | 32.5 | 19.4 | −17.1 | 28 |
| TYR | CA | B | 92 | 32.4 | 18.0 | −17.5 | 25 |
| TYR | CB | B | 92 | 31.2 | 17.3 | −16.8 | 23 |
| TYR | CG | B | 92 | 30.9 | 15.9 | −17.2 | 26 |
| TYR | CD1 | B | 92 | 31.9 | 14.9 | −17.5 | 24 |
| TYR | CE1 | B | 92 | 31.6 | 13.6 | −17.7 | 22 |
| TYR | CD2 | B | 92 | 29.6 | 15.4 | −17.2 | 21 |
| TYR | CE2 | B | 92 | 29.3 | 14.1 | −17.5 | 26 |
| TYR | CZ | B | 92 | 30.3 | 13.2 | −17.7 | 24 |
| TYR | OH | B | 92 | 29.9 | 11.9 | −18.0 | 27 |
| TYR | C | B | 92 | 32.4 | 17.9 | −19.0 | 26 |
| TYR | O | B | 92 | 31.5 | 18.4 | −19.6 | 24 |
| GLN | N | B | 93 | 33.4 | 17.2 | −19.6 | 25 |
| GLN | CA | B | 93 | 33.4 | 17.0 | −21.0 | 30 |
| GLN | CB | B | 93 | 34.7 | 17.5 | −21.7 | 30 |
| GLN | CG | B | 93 | 35.0 | 18.9 | −21.4 | 39 |
| GLN | CD | B | 93 | 36.3 | 19.3 | −22.1 | 38 |
| GLN | OE1 | B | 93 | 37.3 | 19.6 | −21.5 | 45 |
| GLN | NE2 | B | 93 | 36.3 | 19.3 | −23.4 | 35 |
| GLN | C | B | 93 | 33.1 | 15.6 | −21.4 | 28 |
| GLN | O | B | 93 | 34.0 | 14.7 | −21.4 | 26 |
| ARG | N | B | 94 | 31.8 | 15.3 | −21.6 | 24 |
| ARG | CA | B | 94 | 31.3 | 14.0 | −21.9 | 30 |
| ARG | CB | B | 94 | 29.8 | 14.1 | −22.1 | 29 |
| ARG | CG | B | 94 | 29.1 | 14.4 | −20.8 | 28 |
| ARG | CD | B | 94 | 27.6 | 14.6 | −21.0 | 27 |
| ARG | NE | B | 94 | 27.4 | 15.7 | −21.8 | 30 |
| ARG | CZ | B | 94 | 26.2 | 16.1 | −22.3 | 32 |
| ARG | NH1 | B | 94 | 25.1 | 15.5 | −21.9 | 32 |
| ARG | NH2 | B | 94 | 26.1 | 17.2 | −23.0 | 30 |
| ARG | C | B | 94 | 32.0 | 13.3 | −23.1 | 32 |
| ARG | O | B | 94 | 32.2 | 12.1 | −23.0 | 30 |
| GLN | N | B | 95 | 32.4 | 14.0 | −24.1 | 35 |
| GLN | CA | B | 95 | 33.0 | 13.4 | −25.3 | 37 |
| GLN | CB | B | 95 | 33.0 | 14.4 | −26.5 | 43 |
| GLN | CG | B | 95 | 31.6 | 14.8 | −26.9 | 55 |
| GLN | CD | B | 95 | 31.7 | 15.8 | −28.1 | 62 |
| GLN | OE1 | B | 95 | 31.2 | 15.5 | −29.2 | 65 |
| GLN | NE2 | B | 95 | 32.2 | 17.0 | −27.8 | 65 |
| GLN | C | B | 95 | 34.4 | 12.9 | −25.0 | 36 |
| GLN | O | B | 95 | 35.0 | 12.2 | −25.8 | 34 |
| LEU | N | B | 96 | 35.0 | 13.3 | −23.8 | 33 |
| LEU | CA | B | 96 | 36.3 | 12.9 | −23.5 | 30 |
| LEU | CB | B | 96 | 37.1 | 14.0 | −22.9 | 28 |
| LEU | CG | B | 96 | 37.3 | 15.2 | −23.8 | 31 |
| LEU | CD1 | B | 96 | 38.2 | 16.3 | −23.2 | 25 |
| LEU | CD2 | B | 96 | 38.0 | 14.7 | −25.1 | 30 |
| LEU | C | B | 96 | 36.3 | 11.7 | −22.5 | 28 |
| LEU | O | B | 96 | 37.4 | 11.2 | −22.1 | 31 |
| SER | N | B | 97 | 35.1 | 11.2 | −22.2 | 28 |
| SER | CA | B | 97 | 35.0 | 10.0 | −21.3 | 28 |
| SER | CB | B | 97 | 34.1 | 10.3 | −20.1 | 22 |
| SER | OG | B | 97 | 34.0 | 9.2 | −19.3 | 28 |
| SER | C | B | 97 | 34.6 | 8.7 | −22.0 | 28 |
| SER | O | B | 97 | 33.5 | 8.7 | −22.6 | 25 |
| SER | N | B | 98 | 35.4 | 7.7 | −22.0 | 31 |
| SER | CA | B | 98 | 35.1 | 6.5 | −22.6 | 32 |
| SER | CB | B | 98 | 36.3 | 5.6 | −22.8 | 32 |
| SER | OG | B | 98 | 36.9 | 5.3 | −21.6 | 40 |
| SER | C | B | 98 | 34.0 | 5.7 | −21.9 | 31 |
| SER | O | B | 98 | 33.3 | 4.8 | −22.4 | 37 |
| THR | N | B | 99 | 33.8 | 6.0 | −20.6 | 29 |
| THR | CA | B | 99 | 32.8 | 5.4 | −19.8 | 27 |
| THR | CB | B | 99 | 33.4 | 5.1 | −18.4 | 28 |
| THR | OG1 | B | 99 | 33.8 | 6.4 | −17.8 | 29 |
| THR | CG2 | B | 99 | 34.5 | 4.1 | −18.4 | 32 |
| THR | C | B | 99 | 31.4 | 6.1 | −19.7 | 27 |
| THR | O | B | 99 | 30.5 | 5.6 | −19.1 | 29 |
| TYR | N | B | 100 | 31.4 | 7.2 | −20.4 | 27 |
| TYR | CA | B | 100 | 30.1 | 7.9 | −20.6 | 27 |
| TYR | CB | B | 100 | 30.3 | 9.3 | −21.2 | 29 |
| TYR | CG | B | 100 | 29.0 | 10.0 | −21.5 | 33 |
| TYR | CD1 | B | 100 | 28.2 | 10.5 | −20.5 | 31 |
| TYR | CE1 | B | 100 | 27.0 | 11.1 | −20.8 | 32 |
| TYR | CD2 | B | 100 | 28.6 | 10.1 | −22.8 | 34 |
| TYR | CE2 | B | 100 | 27.4 | 10.7 | −23.1 | 33 |
| TYR | CZ | B | 100 | 26.6 | 11.2 | −22.1 | 35 |
| TYR | OH | B | 100 | 25.3 | 11.8 | −22.4 | 38 |
| TYR | C | B | 100 | 29.0 | 7.2 | −21.3 | 27 |
| TYR | O | B | 100 | 29.1 | 6.7 | −22.4 | 28 |
| ARG | N | B | 101 | 27.8 | 7.2 | −20.7 | 28 |
| ARG | CA | B | 101 | 26.6 | 6.6 | −21.2 | 32 |
| ARG | CB | B | 101 | 26.1 | 5.4 | −20.5 | 35 |
| ARG | CG | B | 101 | 26.9 | 4.2 | −20.4 | 42 |
| ARG | CD | B | 101 | 26.1 | 3.2 | −19.6 | 45 |
| ARG | NE | B | 101 | 26.8 | 1.9 | −19.4 | 47 |
| ARG | CZ | B | 101 | 26.4 | 1.0 | −18.5 | 50 |
| ARG | NH1 | B | 101 | 25.4 | 1.3 | −17.7 | 51 |
| ARG | NH2 | B | 101 | 27.1 | −0.1 | −18.3 | 57 |
| ARG | C | B | 101 | 25.5 | 7.7 | −21.3 | 32 |
| ARG | O | B | 101 | 25.2 | 8.3 | −20.2 | 32 |
| ASP | N | B | 102 | 24.9 | 7.8 | −22.4 | 27 |
| ASP | CA | B | 102 | 23.9 | 8.8 | −22.6 | 31 |
| ASP | CB | B | 102 | 23.9 | 9.4 | −24.0 | 32 |
| ASP | CG | B | 102 | 22.8 | 10.5 | −24.2 | 35 |
| ASP | OD1 | B | 102 | 22.0 | 10.8 | −23.3 | 37 |
| ASP | OD2 | B | 102 | 22.8 | 11.1 | −25.3 | 37 |
| ASP | C | B | 102 | 22.5 | 8.2 | −22.3 | 29 |
| ASP | O | B | 102 | 22.2 | 7.2 | −22.9 | 31 |
| LEU | N | B | 103 | 21.8 | 8.7 | −21.3 | 28 |
| LEU | CA | B | 103 | 20.4 | 8.2 | −21.0 | 31 |
| LEU | CB | B | 103 | 20.1 | 8.4 | −19.5 | 28 |
| LEU | CG | B | 103 | 20.9 | 7.6 | −18.5 | 31 |
| LEU | CD1 | B | 103 | 20.7 | 7.9 | −17.1 | 29 |
| LEU | CD2 | B | 103 | 20.7 | 6.1 | −18.8 | 26 |
| LEU | C | B | 103 | 19.4 | 8.7 | −21.9 | 32 |
| LEU | O | B | 103 | 18.2 | 8.3 | −21.7 | 28 |
| ARG | N | B | 104 | 19.7 | 9.6 | −22.8 | 38 |
| ARG | CA | B | 104 | 18.8 | 10.1 | −23.8 | 45 |
| ARG | CB | B | 104 | 18.4 | 8.9 | −24.8 | 46 |
| ARG | CG | B | 104 | 19.5 | 8.4 | −25.6 | 53 |
| ARG | CD | B | 104 | 19.2 | 7.3 | −26.5 | 60 |
| ARG | NE | B | 104 | 18.1 | 7.6 | −27.5 | 70 |
| ARG | CZ | B | 104 | 16.8 | 7.3 | −27.3 | 74 |
| ARG | NH1 | B | 104 | 16.4 | 6.7 | −26.2 | 74 |
| ARG | NH2 | B | 104 | 16.0 | 7.7 | −28.2 | 75 |
| ARG | C | B | 104 | 17.5 | 10.7 | −23.1 | 44 |
| ARG | O | B | 104 | 16.4 | 10.6 | −23.7 | 47 |
| LYS | N | B | 105 | 17.8 | 11.4 | −22.0 | 42 |
| LYS | CA | B | 105 | 16.7 | 12.0 | −21.3 | 41 |
| LYS | CB | B | 105 | 15.9 | 11.0 | −20.4 | 42 |
| LYS | CG | B | 105 | 14.8 | 11.5 | −19.6 | 48 |
| LYS | CD | B | 105 | 14.1 | 10.3 | −18.8 | 54 |
| LYS | CE | B | 105 | 15.1 | 9.7 | −17.9 | 55 |
| LYS | NZ | B | 105 | 14.5 | 8.6 | −17.1 | 58 |
| LYS | C | B | 105 | 17.0 | 13.3 | −20.5 | 40 |
| LYS | O | B | 105 | 18.0 | 13.4 | −19.8 | 40 |
| GLY | N | B | 106 | 16.2 | 14.3 | −20.7 | 38 |
| GLY | CA | B | 106 | 16.4 | 15.6 | −20.1 | 33 |
| GLY | C | B | 106 | 15.9 | 15.7 | −18.6 | 32 |
| GLY | O | B | 106 | 15.1 | 14.9 | −18.2 | 33 |
| VAL | N | B | 107 | 16.5 | 16.6 | −17.9 | 30 |
| VAL | CA | B | 107 | 16.2 | 16.9 | −16.5 | 27 |
| VAL | CB | B | 107 | 17.1 | 16.1 | −15.6 | 26 |
| VAL | CG1 | B | 107 | 18.6 | 16.5 | −15.8 | 23 |
| VAL | CG2 | B | 107 | 16.7 | 16.3 | −14.1 | 26 |
| VAL | C | B | 107 | 16.2 | 18.3 | −16.1 | 26 |
| VAL | O | B | 107 | 17.0 | 19.1 | −16.6 | 30 |
| TYR | N | B | 108 | 15.2 | 18.7 | −15.3 | 28 |
| TYR | CA | B | 108 | 15.0 | 20.1 | −14.8 | 26 |
| TYR | CB | B | 108 | 13.7 | 20.7 | −15.4 | 27 |
| TYR | CG | B | 108 | 13.5 | 22.1 | −14.9 | 29 |
| TYR | CD1 | B | 108 | 14.3 | 23.1 | −15.2 | 27 |
| TYR | CE1 | B | 108 | 14.1 | 24.4 | −14.8 | 28 |
| TYR | CD2 | B | 108 | 12.4 | 22.4 | −14.1 | 29 |
| TYR | CE2 | B | 108 | 12.1 | 23.7 | −13.6 | 29 |
| TYR | CZ | B | 108 | 13.0 | 24.7 | −13.9 | 27 |
| TYR | OH | B | 108 | 12.8 | 26.0 | −13.5 | 28 |
| TYR | C | B | 108 | 14.9 | 20.1 | −13.3 | 27 |
| TYR | O | B | 108 | 14.1 | 19.5 | −12.7 | 31 |
| VAL | N | B | 109 | 15.9 | 20.9 | −12.7 | 27 |
| VAL | CA | B | 109 | 15.9 | 21.0 | −11.3 | 23 |
| VAL | CB | B | 109 | 17.1 | 20.2 | −10.7 | 24 |
| VAL | CG1 | B | 109 | 17.1 | 20.4 | −9.1 | 21 |
| VAL | CG2 | B | 109 | 17.0 | 18.7 | −11.0 | 24 |
| VAL | C | B | 109 | 15.9 | 22.4 | −10.7 | 22 |
| VAL | O | B | 109 | 16.8 | 23.2 | −10.8 | 23 |
| PRO | N | B | 110 | 14.7 | 22.8 | −10.1 | 24 |
| PRO | CD | B | 110 | 13.4 | 22.1 | −10.2 | 21 |
| PRO | CA | B | 110 | 14.6 | 24.1 | −9.5 | 25 |
| PRO | CB | B | 110 | 13.2 | 24.5 | −9.9 | 24 |
| PRO | CG | B | 110 | 12.4 | 23.2 | −9.6 | 22 |
| PRO | C | B | 110 | 14.8 | 24.1 | −8.0 | 24 |
| PRO | O | B | 110 | 14.2 | 23.2 | −7.3 | 26 |
| TYR | N | B | 111 | 15.6 | 25.0 | −7.4 | 24 |
| TYR | CA | B | 111 | 15.8 | 25.0 | −6.0 | 23 |
| TYR | CB | B | 111 | 17.4 | 25.1 | −5.7 | 22 |
| TYR | CG | B | 111 | 18.2 | 24.0 | −6.3 | 20 |
| TYR | CD1 | B | 111 | 18.3 | 22.7 | −5.6 | 22 |
| TYR | CE1 | B | 111 | 19.1 | 21.7 | −6.2 | 23 |
| TYR | CD2 | B | 111 | 18.8 | 24.1 | −7.5 | 20 |
| TYR | CE2 | B | 111 | 19.6 | 23.1 | −8.1 | 24 |
| TYR | CZ | B | 111 | 19.7 | 21.9 | −7.4 | 25 |
| TYR | OH | B | 111 | 20.5 | 20.9 | −7.9 | 24 |
| TYR | C | B | 111 | 15.1 | 26.2 | −5.4 | 26 |
| TYR | O | B | 111 | 14.4 | 26.9 | −6.1 | 22 |
| THR | N | B | 112 | 15.4 | 26.5 | −4.1 | 23 |
| THR | CA | B | 112 | 14.7 | 27.7 | −3.5 | 23 |
| THR | CB | B | 112 | 14.9 | 27.7 | −2.0 | 23 |
| THR | OG1 | B | 112 | 14.4 | 26.5 | −1.4 | 21 |
| THR | CG2 | B | 112 | 14.4 | 29.0 | −1.4 | 22 |
| THR | C | B | 112 | 15.3 | 28.9 | −4.2 | 28 |
| THR | O | B | 112 | 14.6 | 29.9 | −4.4 | 29 |
| GLN | N | B | 113 | 16.6 | 28.9 | −4.5 | 31 |
| GLN | CA | B | 113 | 17.3 | 30.0 | −5.2 | 33 |
| GLN | CB | B | 113 | 18.1 | 30.8 | −4.2 | 40 |
| GLN | CG | B | 113 | 17.3 | 31.4 | −3.1 | 53 |
| GLN | CD | B | 113 | 16.3 | 32.4 | −3.6 | 59 |
| GLN | OE1 | B | 113 | 16.2 | 32.7 | −4.8 | 64 |
| GLN | NE2 | B | 113 | 15.5 | 33.0 | −2.7 | 60 |
| GLN | C | B | 113 | 18.2 | 29.3 | −6.2 | 31 |
| GLN | O | B | 113 | 19.1 | 28.5 | −5.9 | 25 |
| GLY | N | B | 114 | 17.9 | 29.5 | −7.5 | 26 |
| GLY | CA | B | 114 | 18.7 | 28.9 | −8.6 | 27 |
| GLY | C | B | 114 | 18.0 | 27.8 | −9.2 | 26 |
| GLY | O | B | 114 | 17.2 | 27.1 | −8.6 | 26 |
| LYS | N | B | 115 | 18.3 | 27.5 | −10.5 | 29 |
| LYS | CA | B | 115 | 17.6 | 26.5 | −11.3 | 27 |
| LYS | CB | B | 115 | 16.3 | 26.9 | −11.8 | 32 |
| LYS | CG | B | 115 | 16.3 | 28.1 | −12.7 | 39 |
| LYS | CD | B | 115 | 15.0 | 28.5 | −13.3 | 47 |
| LYS | CE | B | 115 | 14.0 | 28.9 | −12.2 | 49 |
| LYS | NZ | B | 115 | 12.7 | 29.4 | −12.8 | 55 |
| LYS | C | B | 115 | 18.5 | 26.1 | −12.4 | 26 |
| LYS | O | B | 115 | 19.3 | 26.9 | −12.9 | 27 |
| TRP | N | B | 116 | 18.4 | 24.8 | −12.9 | 25 |
| TRP | CA | B | 116 | 19.2 | 24.4 | −14.0 | 24 |
| TRP | CB | B | 116 | 20.6 | 24.0 | −13.6 | 23 |
| TRP | CG | B | 116 | 20.7 | 22.9 | −12.6 | 25 |
| TRP | CD2 | B | 116 | 20.4 | 21.5 | −12.9 | 24 |
| TRP | CE2 | B | 116 | 20.6 | 20.8 | −11.6 | 24 |
| TRP | CE3 | B | 116 | 20.1 | 20.7 | −14.0 | 28 |
| TRP | CD1 | B | 116 | 21.0 | 23.0 | −11.3 | 23 |
| TRP | NE1 | B | 116 | 20.9 | 21.8 | −10.7 | 25 |
| TRP | CZ2 | B | 116 | 20.3 | 19.4 | −11.5 | 22 |
| TRP | CZ3 | B | 116 | 19.9 | 19.3 | −13.8 | 24 |
| TRP | CH2 | B | 116 | 20.0 | 18.7 | −12.6 | 25 |
| TRP | C | B | 116 | 18.5 | 23.3 | −14.9 | 25 |
| TRP | O | B | 116 | 17.8 | 22.5 | −14.3 | 26 |
| GLU | N | B | 117 | 18.8 | 23.3 | −16.2 | 32 |
| GLU | CA | B | 117 | 18.3 | 22.3 | −17.1 | 36 |
| GLU | CB | B | 117 | 17.6 | 22.9 | −18.2 | 41 |
| GLU | CG | B | 117 | 17.0 | 21.8 | −19.2 | 54 |
| GLU | CD | B | 117 | 16.2 | 22.4 | −20.3 | 61 |
| GLU | OE1 | B | 117 | 15.0 | 22.2 | −20.5 | 65 |
| GLU | OE2 | B | 117 | 16.9 | 23.2 | −21.1 | 62 |
| GLU | C | B | 117 | 19.5 | 21.5 | −17.5 | 33 |
| GLU | O | B | 117 | 20.6 | 22.0 | −17.8 | 30 |
| GLY | N | B | 118 | 19.4 | 20.1 | −17.6 | 31 |
| GLY | CA | B | 118 | 20.5 | 19.3 | −18.0 | 32 |
| GLY | C | B | 118 | 20.1 | 18.1 | −18.8 | 30 |
| GLY | O | B | 118 | 18.9 | 17.8 | −19.2 | 29 |
| GLU | N | B | 119 | 21.1 | 17.2 | −19.0 | 28 |
| GLU | CA | B | 119 | 20.9 | 16.0 | −19.8 | 28 |
| GLU | CB | B | 119 | 21.6 | 16.0 | −21.1 | 31 |
| GLU | CG | B | 119 | 21.2 | 17.3 | −21.9 | 37 |
| GLU | CD | B | 119 | 21.9 | 17.3 | −23.3 | 42 |
| GLU | OE1 | B | 119 | 21.3 | 17.6 | −24.3 | 50 |
| GLU | OE2 | B | 119 | 23.1 | 17.1 | −23.3 | 43 |
| GLU | C | B | 119 | 21.4 | 14.7 | −19.0 | 26 |
| GLU | O | B | 119 | 22.5 | 14.7 | −18.6 | 26 |
| LEU | N | B | 120 | 20.5 | 13.8 | −18.7 | 25 |
| LEU | CA | B | 120 | 20.8 | 12.6 | −17.9 | 25 |
| LEU | CB | B | 120 | 19.6 | 11.8 | −17.5 | 24 |
| LEU | CG | B | 120 | 18.7 | 12.6 | −16.5 | 25 |
| LEU | CD1 | B | 120 | 17.4 | 11.8 | −16.2 | 27 |
| LEU | CD2 | B | 120 | 19.4 | 13.0 | −15.2 | 28 |
| LEU | C | B | 120 | 21.8 | 11.7 | −18.6 | 27 |
| LEU | O | B | 120 | 21.8 | 11.5 | −19.8 | 31 |
| GLY | N | B | 121 | 22.6 | 11.0 | −17.7 | 29 |
| GLY | CA | B | 121 | 23.6 | 10.1 | −18.2 | 30 |
| GLY | C | B | 121 | 24.2 | 9.3 | −17.0 | 32 |
| GLY | O | B | 121 | 23.8 | 9.5 | −15.9 | 29 |
| THR | N | B | 122 | 25.1 | 8.4 | −17.3 | 31 |
| THR | CA | B | 122 | 25.8 | 7.6 | −16.2 | 29 |
| THR | CB | B | 122 | 25.3 | 6.2 | −16.1 | 26 |
| THR | OG1 | B | 122 | 25.6 | 5.5 | −17.3 | 33 |
| THR | CG2 | B | 122 | 23.8 | 6.1 | −15.8 | 26 |
| THR | C | B | 122 | 27.3 | 7.6 | −16.5 | 29 |
| THR | O | B | 122 | 27.6 | 7.8 | −17.7 | 28 |
| ASP | N | B | 123 | 28.1 | 7.5 | −15.5 | 28 |
| ASP | CA | B | 123 | 29.5 | 7.5 | −15.8 | 25 |
| ASP | CB | B | 123 | 30.0 | 8.8 | −16.4 | 25 |
| ASP | CG | B | 123 | 31.4 | 8.8 | −17.0 | 26 |
| ASP | OD1 | B | 123 | 32.1 | 7.8 | −16.8 | 21 |
| ASP | OD2 | B | 123 | 31.7 | 9.8 | −17.6 | 21 |
| ASP | C | B | 123 | 30.2 | 7.1 | −14.5 | 27 |
| ASP | O | B | 123 | 29.6 | 7.1 | −13.4 | 27 |
| LEU | N | B | 124 | 31.5 | 6.8 | −14.5 | 26 |
| LEU | CA | B | 124 | 32.3 | 6.4 | −13.3 | 27 |
| LEU | CB | B | 124 | 33.6 | 5.7 | −13.6 | 25 |
| LEU | CG | B | 124 | 33.3 | 4.4 | −14.5 | 26 |
| LEU | CD1 | B | 124 | 34.6 | 3.7 | −14.8 | 21 |
| LEU | CD2 | B | 124 | 32.4 | 3.4 | −13.8 | 25 |
| LEU | C | B | 124 | 32.6 | 7.6 | −12.5 | 29 |
| LEU | O | B | 124 | 33.0 | 8.7 | −13.0 | 30 |
| VAL | N | B | 125 | 32.3 | 7.5 | −11.2 | 28 |
| VAL | CA | B | 125 | 32.4 | 8.6 | −10.2 | 23 |
| VAL | CB | B | 125 | 31.1 | 9.2 | −9.8 | 22 |
| VAL | CG1 | B | 125 | 31.3 | 10.4 | −8.8 | 19 |
| VAL | CG2 | B | 125 | 30.3 | 9.7 | −11.0 | 14 |
| VAL | C | B | 125 | 33.2 | 8.2 | −8.9 | 26 |
| VAL | O | B | 125 | 32.9 | 7.1 | −8.4 | 27 |
| SER | N | B | 126 | 34.1 | 9.0 | −8.5 | 21 |
| SER | CA | B | 126 | 34.9 | 8.8 | −7.3 | 28 |
| SER | CB | B | 126 | 36.2 | 8.1 | −7.6 | 22 |
| SER | OG | B | 126 | 37.1 | 8.9 | −8.4 | 31 |
| SER | C | B | 126 | 35.2 | 10.0 | −6.5 | 28 |
| SER | O | B | 126 | 35.2 | 11.1 | −7.1 | 27 |
| ILE | N | B | 127 | 35.4 | 9.9 | −5.2 | 27 |
| ILE | CA | B | 127 | 35.7 | 11.0 | −4.3 | 27 |
| ILE | CB | B | 127 | 34.7 | 11.0 | −3.1 | 22 |
| ILE | CG2 | B | 127 | 35.1 | 12.2 | −2.2 | 23 |
| ILE | CG1 | B | 127 | 33.3 | 11.1 | −3.6 | 23 |
| ILE | CD1 | B | 127 | 32.3 | 11.1 | −2.4 | 27 |
| ILE | C | B | 127 | 37.1 | 10.7 | −3.9 | 28 |
| ILE | O | B | 127 | 37.4 | 9.9 | −3.0 | 28 |
| PRO | N | B | 128 | 38.1 | 11.5 | −4.4 | 26 |
| PRO | CD | B | 128 | 38.0 | 12.3 | −5.6 | 24 |
| PRO | CA | B | 128 | 39.5 | 11.4 | −4.1 | 27 |
| PRO | CB | B | 128 | 40.1 | 12.6 | −4.8 | 26 |
| PRO | CG | B | 128 | 39.5 | 12.5 | −6.1 | 25 |
| PRO | C | B | 128 | 39.8 | 11.3 | −2.6 | 28 |
| PRO | O | B | 128 | 40.7 | 10.5 | −2.1 | 34 |
| HIS | N | B | 129 | 39.2 | 12.2 | −1.8 | 28 |
| HIS | CA | B | 129 | 39.4 | 12.3 | −0.4 | 28 |
| HIS | CB | B | 129 | 39.7 | 13.7 | 0.0 | 28 |
| HIS | CG | B | 129 | 40.9 | 14.3 | −0.6 | 32 |
| HIS | CD2 | B | 129 | 41.0 | 15.2 | −1.6 | 34 |
| HIS | ND1 | B | 129 | 42.2 | 13.9 | −0.3 | 29 |
| HIS | CE1 | B | 129 | 43.0 | 14.5 | −1.1 | 32 |
| HIS | NE2 | B | 129 | 42.4 | 15.3 | −1.9 | 35 |
| HIS | C | B | 129 | 38.3 | 11.6 | 0.4 | 27 |
| HIS | O | B | 129 | 37.7 | 12.2 | 1.3 | 28 |
| GLY | N | B | 130 | 37.8 | 10.5 | −0.1 | 31 |
| GLY | CA | B | 130 | 36.8 | 9.7 | 0.5 | 32 |
| GLY | C | B | 130 | 37.2 | 8.3 | 0.4 | 29 |
| GLY | O | B | 130 | 38.4 | 8.0 | 0.4 | 27 |
| PRO | N | B | 131 | 36.2 | 7.3 | 0.3 | 31 |
| PRO | CD | B | 131 | 34.8 | 7.5 | 0.0 | 31 |
| PRO | CA | B | 131 | 36.6 | 5.9 | 0.2 | 31 |
| PRO | CB | B | 131 | 35.2 | 5.2 | 0.4 | 31 |
| PRO | CG | B | 131 | 34.4 | 6.1 | −0.5 | 35 |
| PRO | C | B | 131 | 37.3 | 5.5 | −1.1 | 29 |
| PRO | O | B | 131 | 37.0 | 6.2 | −2.1 | 29 |
| ASN | N | B | 132 | 38.2 | 4.6 | −1.0 | 29 |
| ASN | CA | B | 132 | 38.9 | 4.1 | −2.2 | 33 |
| ASN | CB | B | 132 | 40.1 | 3.2 | −1.8 | 36 |
| ASN | CG | B | 132 | 39.7 | 2.0 | −1.1 | 38 |
| ASN | OD1 | B | 132 | 38.6 | 1.9 | −0.6 | 42 |
| ASN | ND2 | B | 132 | 40.6 | 1.0 | −1.1 | 37 |
| ASN | C | B | 132 | 38.0 | 3.4 | −3.2 | 32 |
| ASN | O | B | 132 | 38.4 | 2.3 | −3.6 | 35 |
| VAL | N | B | 133 | 36.9 | 4.0 | −3.6 | 28 |
| VAL | CA | B | 133 | 36.0 | 3.3 | −4.5 | 27 |
| VAL | CB | B | 133 | 34.8 | 2.7 | −3.8 | 25 |
| VAL | CG1 | B | 133 | 35.2 | 1.7 | −2.8 | 23 |
| VAL | CG2 | B | 133 | 33.9 | 3.8 | −3.2 | 27 |
| VAL | C | B | 133 | 35.5 | 4.2 | −5.7 | 28 |
| VAL | O | B | 133 | 35.4 | 5.4 | −5.6 | 28 |
| THR | N | B | 134 | 35.3 | 3.5 | −6.8 | 26 |
| THR | CA | B | 134 | 34.8 | 4.1 | −8.0 | 23 |
| THR | CB | B | 134 | 35.7 | 4.0 | −9.2 | 23 |
| THR | OG1 | B | 134 | 37.0 | 4.6 | −8.9 | 23 |
| THR | CG2 | B | 134 | 35.1 | 4.6 | −10.5 | 22 |
| THR | C | B | 134 | 33.4 | 3.4 | −8.3 | 26 |
| THR | O | B | 134 | 33.3 | 2.2 | −8.2 | 26 |
| VAL | N | B | 135 | 32.4 | 4.2 | −8.6 | 27 |
| VAL | CA | B | 135 | 31.1 | 3.7 | −8.9 | 28 |
| VAL | CB | B | 135 | 30.2 | 3.8 | −7.6 | 30 |
| VAL | CG1 | B | 135 | 30.8 | 3.0 | −6.5 | 29 |
| VAL | CG2 | B | 135 | 29.9 | 5.2 | −7.2 | 32 |
| VAL | C | B | 135 | 30.4 | 4.4 | −10.0 | 30 |
| VAL | O | B | 135 | 30.6 | 5.6 | −10.3 | 29 |
| ARG | N | B | 136 | 29.6 | 3.6 | −10.8 | 30 |
| ARG | CA | B | 136 | 28.8 | 4.2 | −11.8 | 28 |
| ARG | CB | B | 136 | 28.4 | 3.3 | −13.0 | 27 |
| ARG | CG | B | 136 | 27.5 | 3.8 | −14.0 | 25 |
| ARG | CD | B | 136 | 27.3 | 2.9 | −15.2 | 24 |
| ARG | NE | B | 136 | 28.5 | 2.5 | −15.8 | 20 |
| ARG | CZ | B | 136 | 29.1 | 3.3 | −16.7 | 24 |
| ARG | NH1 | B | 136 | 28.5 | 4.4 | −17.1 | 25 |
| ARG | NH2 | B | 136 | 30.3 | 2.9 | −17.3 | 23 |
| ARG | C | B | 136 | 27.6 | 4.9 | −11.2 | 27 |
| ARG | O | B | 136 | 26.9 | 4.3 | −10.4 | 28 |
| ALA | N | B | 137 | 27.4 | 6.1 | −11.5 | 23 |
| ALA | CA | B | 137 | 26.2 | 6.9 | −11.0 | 24 |
| ALA | CB | B | 137 | 26.7 | 7.8 | −9.8 | 15 |
| ALA | C | B | 137 | 25.6 | 7.7 | −12.1 | 23 |
| ALA | O | B | 137 | 26.1 | 8.0 | −13.1 | 23 |
| ASN | N | B | 138 | 24.3 | 8.1 | −11.8 | 21 |
| ASN | CA | B | 138 | 23.6 | 9.0 | −12.7 | 23 |
| ASN | CB | B | 138 | 22.1 | 9.0 | −12.3 | 23 |
| ASN | CG | B | 138 | 21.5 | 7.7 | −12.4 | 22 |
| ASN | OD1 | B | 138 | 21.6 | 7.0 | −13.5 | 25 |
| ASN | ND2 | B | 138 | 20.8 | 7.2 | −11.4 | 22 |
| ASN | C | B | 138 | 24.2 | 10.4 | −12.5 | 28 |
| ASN | O | B | 138 | 24.6 | 10.8 | −11.4 | 27 |
| ILE | N | B | 139 | 24.4 | 11.1 | −13.7 | 26 |
| ILE | CA | B | 139 | 24.9 | 12.4 | −13.7 | 26 |
| ILE | CB | B | 139 | 26.4 | 12.4 | −14.2 | 26 |
| ILE | CG2 | B | 139 | 26.9 | 13.8 | −14.3 | 27 |
| ILE | CG1 | B | 139 | 27.3 | 11.5 | −13.3 | 22 |
| ILE | CD1 | B | 139 | 28.7 | 11.5 | −13.8 | 25 |
| ILE | C | B | 139 | 24.1 | 13.3 | −14.6 | 30 |
| ILE | O | B | 139 | 23.9 | 13.0 | −15.7 | 29 |
| ALA | N | B | 140 | 23.6 | 14.4 | −14.0 | 29 |
| ALA | CA | B | 140 | 22.9 | 15.4 | −14.8 | 27 |
| ALA | CB | B | 140 | 21.9 | 16.1 | −13.9 | 25 |
| ALA | C | B | 140 | 23.9 | 16.4 | −15.4 | 29 |
| ALA | O | B | 140 | 24.6 | 17.1 | −14.6 | 28 |
| ALA | N | B | 141 | 24.0 | 16.4 | −16.7 | 26 |
| ALA | CA | B | 141 | 25.0 | 17.2 | −17.3 | 28 |
| ALA | CB | B | 141 | 25.3 | 16.7 | −18.7 | 25 |
| ALA | C | B | 141 | 24.2 | 18.6 | −17.5 | 28 |
| ALA | O | B | 141 | 23.3 | 18.7 | −18.3 | 33 |
| ILE | N | B | 142 | 24.7 | 19.6 | −16.8 | 27 |
| ILE | CA | B | 142 | 24.1 | 20.9 | −16.8 | 26 |
| ILE | CB | B | 142 | 24.3 | 21.7 | −15.6 | 24 |
| ILE | CG2 | B | 142 | 23.7 | 23.1 | −15.7 | 22 |
| ILE | CG1 | B | 142 | 23.8 | 21.0 | −14.3 | 19 |
| ILE | CD1 | B | 142 | 24.1 | 21.7 | −13.0 | 24 |
| ILE | C | B | 142 | 24.4 | 21.7 | −18.1 | 27 |
| ILE | O | B | 142 | 25.6 | 22.0 | −18.3 | 23 |
| THR | N | B | 143 | 23.4 | 22.0 | −18.9 | 28 |
| THR | CA | B | 143 | 23.6 | 22.7 | −20.2 | 29 |
| THR | CB | B | 143 | 22.9 | 22.0 | −21.3 | 27 |
| THR | OG1 | B | 143 | 21.5 | 21.8 | −21.1 | 24 |
| THR | CG2 | B | 143 | 23.6 | 20.6 | −21.6 | 25 |
| THR | C | B | 143 | 23.1 | 24.2 | −20.1 | 30 |
| THR | O | B | 143 | 23.5 | 25.0 | −20.9 | 30 |
| GLU | N | B | 144 | 22.2 | 24.5 | −19.2 | 35 |
| GLU | CA | B | 144 | 21.7 | 25.8 | −19.0 | 39 |
| GLU | CB | B | 144 | 20.4 | 26.0 | −19.8 | 47 |
| GLU | CG | B | 144 | 20.7 | 25.9 | −21.3 | 61 |
| GLU | CD | B | 144 | 19.4 | 26.2 | −22.1 | 72 |
| GLU | OE1 | B | 144 | 18.9 | 25.3 | −22.8 | 77 |
| GLU | OE2 | B | 144 | 18.8 | 27.3 | −21.9 | 76 |
| GLU | C | B | 144 | 21.4 | 26.0 | −17.5 | 34 |
| GLU | O | B | 144 | 20.9 | 25.0 | −16.9 | 34 |
| SER | N | B | 145 | 21.7 | 27.2 | −17.0 | 30 |
| SER | CA | B | 145 | 21.4 | 27.4 | −15.6 | 31 |
| SER | CB | B | 145 | 22.6 | 27.1 | −14.7 | 29 |
| SER | OG | B | 145 | 23.7 | 27.9 | −15.1 | 27 |
| SER | C | B | 145 | 21.0 | 28.9 | −15.3 | 32 |
| SER | O | B | 145 | 21.4 | 29.8 | −16.1 | 31 |
| ASP | N | B | 146 | 20.2 | 29.1 | −14.3 | 31 |
| ASP | CA | B | 146 | 19.7 | 30.4 | −14.0 | 32 |
| ASP | CB | B | 146 | 18.2 | 30.6 | −14.3 | 39 |
| ASP | CG | B | 146 | 17.7 | 32.0 | −14.1 | 46 |
| ASP | OD1 | B | 146 | 18.5 | 32.9 | −13.8 | 45 |
| ASP | OD2 | B | 146 | 16.5 | 32.2 | −14.2 | 52 |
| ASP | C | B | 146 | 19.9 | 30.6 | −12.5 | 29 |
| ASP | O | B | 146 | 19.3 | 30.0 | −11.6 | 28 |
| LYS | N | B | 147 | 20.9 | 31.5 | −12.1 | 23 |
| LYS | CA | B | 147 | 21.2 | 31.8 | −10.7 | 29 |
| LYS | CB | B | 147 | 20.0 | 32.5 | −10.0 | 28 |
| LYS | CG | B | 147 | 19.6 | 33.9 | −10.6 | 38 |
| LYS | CD | B | 147 | 18.5 | 34.5 | −9.9 | 41 |
| LYS | CE | B | 147 | 18.1 | 35.8 | −10.5 | 47 |
| LYS | NZ | B | 147 | 16.9 | 36.4 | −9.8 | 55 |
| LYS | C | B | 147 | 21.7 | 30.6 | −9.9 | 27 |
| LYS | O | B | 147 | 21.5 | 30.6 | −8.7 | 25 |
| PHE | N | B | 148 | 22.2 | 29.6 | −10.6 | 28 |
| PHE | CA | B | 148 | 22.7 | 28.4 | −9.9 | 28 |
| PHE | CB | B | 148 | 22.5 | 27.2 | −10.7 | 29 |
| PHE | CG | B | 148 | 23.0 | 25.9 | −10.1 | 26 |
| PHE | CD1 | B | 148 | 22.3 | 25.4 | −9.0 | 26 |
| PHE | CD2 | B | 148 | 24.1 | 25.2 | −10.6 | 23 |
| PHE | CE1 | B | 148 | 22.8 | 24.2 | −8.4 | 26 |
| PHE | CE2 | B | 148 | 24.6 | 24.1 | −10.0 | 24 |
| PHE | CZ | B | 148 | 23.9 | 23.5 | −8.9 | 26 |
| PHE | C | B | 148 | 24.2 | 28.7 | −9.5 | 25 |
| PHE | O | B | 148 | 24.5 | 28.7 | −8.4 | 28 |
| PHE | N | B | 149 | 25.0 | 28.8 | −10.6 | 28 |
| PHE | CA | B | 149 | 26.4 | 29.0 | −10.4 | 29 |
| PHE | CB | B | 149 | 27.2 | 28.8 | −11.7 | 29 |
| PHE | CG | B | 149 | 27.0 | 27.4 | −12.3 | 29 |
| PHE | CD1 | B | 149 | 26.4 | 27.2 | −13.5 | 27 |
| PHE | CD2 | B | 149 | 27.4 | 26.3 | −11.5 | 28 |
| PHE | CE1 | B | 149 | 26.3 | 25.9 | −14.0 | 28 |
| PHE | CE2 | B | 149 | 27.2 | 25.0 | −12.0 | 26 |
| PHE | CZ | B | 149 | 26.6 | 24.8 | −13.3 | 27 |
| PHE | C | B | 149 | 26.8 | 30.4 | −9.8 | 31 |
| PHE | O | B | 149 | 26.2 | 31.4 | −10.1 | 30 |
| ILE | N | B | 150 | 27.8 | 30.4 | −8.9 | 32 |
| ILE | CA | B | 150 | 28.3 | 31.6 | −8.2 | 33 |
| ILE | CB | B | 150 | 28.3 | 31.4 | −6.7 | 35 |
| ILE | CG2 | B | 150 | 28.8 | 32.7 | −6.1 | 34 |
| ILE | CG1 | B | 150 | 27.0 | 31.0 | −6.2 | 34 |
| ILE | CD1 | B | 150 | 27.0 | 30.8 | −4.7 | 36 |
| ILE | C | B | 150 | 29.7 | 32.0 | −8.8 | 35 |
| ILE | O | B | 150 | 30.6 | 31.1 | −8.8 | 33 |
| ASN | N | B | 151 | 29.8 | 33.2 | −9.1 | 33 |
| ASN | CA | B | 151 | 31.1 | 33.7 | −9.6 | 32 |
| ASN | CB | B | 151 | 31.0 | 35.2 | −10.0 | 36 |
| ASN | CG | B | 151 | 32.3 | 35.8 | −10.6 | 37 |
| ASN | OD1 | B | 151 | 32.3 | 36.9 | −11.2 | 37 |
| ASN | ND2 | B | 151 | 33.4 | 35.1 | −10.4 | 36 |
| ASN | C | B | 151 | 32.2 | 33.6 | −8.6 | 35 |
| ASN | O | B | 151 | 32.1 | 34.2 | −7.5 | 35 |
| GLY | N | B | 152 | 33.2 | 32.7 | −8.8 | 34 |
| GLY | CA | B | 152 | 34.2 | 32.5 | −7.9 | 31 |
| GLY | C | B | 152 | 34.0 | 31.6 | −6.7 | 34 |
| GLY | O | B | 152 | 34.9 | 31.5 | −5.8 | 31 |
| SER | N | B | 153 | 32.9 | 30.8 | −6.7 | 31 |
| SER | CA | B | 153 | 32.7 | 29.9 | −5.6 | 31 |
| SER | CB | B | 153 | 31.3 | 29.3 | −5.7 | 27 |
| SER | OG | B | 153 | 31.1 | 28.5 | −6.9 | 24 |
| SER | C | B | 153 | 33.7 | 28.7 | −5.6 | 31 |
| SER | O | B | 153 | 33.9 | 28.1 | −4.5 | 30 |
| ASN | N | B | 154 | 34.3 | 28.5 | −6.7 | 26 |
| ASN | CA | B | 154 | 35.3 | 27.5 | −6.9 | 29 |
| ASN | CB | B | 154 | 36.4 | 27.5 | −5.9 | 29 |
| ASN | CG | B | 154 | 37.6 | 26.7 | −6.3 | 35 |
| ASN | OD1 | B | 154 | 37.8 | 26.4 | −7.5 | 38 |
| ASN | ND2 | B | 154 | 38.3 | 26.2 | −5.3 | 37 |
| ASN | C | B | 154 | 34.7 | 26.1 | −7.1 | 28 |
| ASN | O | B | 154 | 35.5 | 25.1 | −7.0 | 29 |
| TRP | N | B | 155 | 33.4 | 26.0 | −7.2 | 24 |
| TRP | CA | B | 155 | 32.8 | 24.7 | −7.4 | 25 |
| TRP | CB | B | 155 | 31.9 | 24.2 | −6.2 | 25 |
| TRP | CG | B | 155 | 30.8 | 25.2 | −5.8 | 28 |
| TRP | CD2 | B | 155 | 29.5 | 25.3 | −6.4 | 23 |
| TRP | CE2 | B | 155 | 28.7 | 26.2 | −5.7 | 27 |
| TRP | CE3 | B | 155 | 28.9 | 24.7 | −7.5 | 24 |
| TRP | CD1 | B | 155 | 30.7 | 26.0 | −4.7 | 27 |
| TRP | NE1 | B | 155 | 29.5 | 26.6 | −4.6 | 26 |
| TRP | CZ2 | B | 155 | 27.4 | 26.6 | −6.0 | 26 |
| TRP | CZ3 | B | 155 | 27.6 | 25.1 | −7.9 | 20 |
| TRP | CH2 | B | 155 | 26.9 | 26.0 | −7.1 | 27 |
| TRP | C | B | 155 | 32.0 | 24.6 | −8.7 | 28 |
| TRP | O | B | 155 | 31.4 | 25.6 | −9.1 | 28 |
| GLU | N | B | 156 | 32.0 | 23.4 | −9.4 | 23 |
| GLU | CA | B | 156 | 31.4 | 23.3 | −10.7 | 22 |
| GLU | CB | B | 156 | 32.3 | 22.8 | −11.8 | 26 |
| GLU | CG | B | 156 | 33.6 | 23.6 | −12.1 | 26 |
| GLU | CD | B | 156 | 34.6 | 23.6 | −10.9 | 24 |
| GLU | OE1 | B | 156 | 34.9 | 24.7 | −10.4 | 23 |
| GLU | OE2 | B | 156 | 35.0 | 22.5 | −10.4 | 24 |
| GLU | C | B | 156 | 30.1 | 22.4 | −10.7 | 24 |
| GLU | O | B | 156 | 29.5 | 22.2 | −11.7 | 22 |
| GLY | N | B | 157 | 29.8 | 21.8 | −9.5 | 24 |
| GLY | CA | B | 157 | 28.7 | 20.9 | −9.5 | 22 |
| GLY | C | B | 157 | 28.0 | 20.8 | −8.1 | 24 |
| GLY | O | B | 157 | 28.3 | 21.6 | −7.2 | 25 |
| ILE | N | B | 158 | 27.0 | 20.0 | −8.0 | 23 |
| ILE | CA | B | 158 | 26.2 | 19.8 | −6.8 | 23 |
| ILE | CB | B | 158 | 24.9 | 20.6 | −6.9 | 26 |
| ILE | CG2 | B | 158 | 24.0 | 20.1 | −8.0 | 21 |
| ILE | CG1 | B | 158 | 24.1 | 20.4 | −5.6 | 22 |
| ILE | CD1 | B | 158 | 22.9 | 21.2 | −5.5 | 21 |
| ILE | C | B | 158 | 26.0 | 18.3 | −6.6 | 26 |
| ILE | O | B | 158 | 25.6 | 17.5 | −7.5 | 24 |
| LEU | N | B | 159 | 26.2 | 17.9 | −5.3 | 21 |
| LEU | CA | B | 159 | 26.0 | 16.5 | −4.9 | 20 |
| LEU | CB | B | 159 | 27.2 | 16.0 | −4.2 | 20 |
| LEU | CG | B | 159 | 27.1 | 14.5 | −3.7 | 22 |
| LEU | CD1 | B | 159 | 26.9 | 13.6 | −4.9 | 19 |
| LEU | CD2 | B | 159 | 28.4 | 14.1 | −3.0 | 16 |
| LEU | C | B | 159 | 24.7 | 16.4 | −4.0 | 25 |
| LEU | O | B | 159 | 24.8 | 16.8 | −2.9 | 24 |
| GLY | N | B | 160 | 23.6 | 15.9 | −4.6 | 21 |
| GLY | CA | B | 160 | 22.4 | 15.8 | −3.8 | 21 |
| GLY | C | B | 160 | 22.4 | 14.6 | −3.0 | 24 |
| GLY | O | B | 160 | 22.3 | 13.4 | −3.5 | 23 |
| LEU | N | B | 161 | 22.5 | 14.8 | −1.7 | 22 |
| LEU | CA | B | 161 | 22.5 | 13.7 | −0.7 | 19 |
| LEU | CB | B | 161 | 23.6 | 14.0 | 0.4 | 21 |
| LEU | CG | B | 161 | 25.0 | 14.1 | −0.2 | 21 |
| LEU | CD1 | B | 161 | 26.0 | 14.6 | 0.9 | 20 |
| LEU | CD2 | B | 161 | 25.4 | 12.8 | −0.8 | 21 |
| LEU | C | B | 161 | 21.2 | 13.2 | −0.1 | 19 |
| LEU | O | B | 161 | 21.2 | 12.3 | 0.7 | 19 |
| ALA | N | B | 162 | 20.1 | 13.8 | −0.4 | 17 |
| ALA | CA | B | 162 | 18.8 | 13.4 | 0.1 | 22 |
| ALA | CB | B | 162 | 17.8 | 14.6 | 0.2 | 20 |
| ALA | C | B | 162 | 18.2 | 12.2 | −0.6 | 21 |
| ALA | O | B | 162 | 18.9 | 11.7 | −1.5 | 20 |
| TYR | N | B | 163 | 17.0 | 11.8 | −0.2 | 26 |
| TYR | CA | B | 163 | 16.4 | 10.6 | −0.8 | 26 |
| TYR | CB | B | 163 | 15.3 | 10.1 | 0.2 | 23 |
| TYR | CG | B | 163 | 15.9 | 9.8 | 1.6 | 26 |
| TYR | CD1 | B | 163 | 16.0 | 10.7 | 2.5 | 23 |
| TYR | CE1 | B | 163 | 16.6 | 10.5 | 3.8 | 27 |
| TYR | CD2 | B | 163 | 16.5 | 8.5 | 1.9 | 26 |
| TYR | CE2 | B | 163 | 17.1 | 8.2 | 3.1 | 23 |
| TYR | CZ | B | 163 | 17.1 | 9.2 | 4.1 | 27 |
| TYR | OH | B | 163 | 17.7 | 9.0 | 5.3 | 24 |
| TYR | C | B | 163 | 15.8 | 10.7 | −2.2 | 27 |
| TYR | O | B | 163 | 15.5 | 11.8 | −2.7 | 24 |
| ALA | N | B | 164 | 15.7 | 9.5 | −2.8 | 26 |
| ALA | CA | B | 164 | 15.2 | 9.5 | −4.2 | 27 |
| ALA | CB | B | 164 | 15.1 | 8.0 | −4.6 | 28 |
| ALA | C | B | 164 | 13.8 | 10.1 | −4.3 | 26 |
| ALA | O | B | 164 | 13.4 | 10.5 | −5.4 | 26 |
| GLU | N | B | 165 | 13.0 | 10.2 | −3.2 | 30 |
| GLU | CA | B | 165 | 11.7 | 10.7 | −3.2 | 31 |
| GLU | CB | B | 165 | 11.1 | 10.8 | −1.8 | 39 |
| GLU | CG | B | 165 | 9.7 | 11.4 | −1.7 | 46 |
| GLU | CD | B | 165 | 8.7 | 10.6 | −2.5 | 53 |
| GLU | OE1 | B | 165 | 7.7 | 10.1 | −1.8 | 57 |
| GLU | OE2 | B | 165 | 8.8 | 10.5 | −3.7 | 57 |
| GLU | C | B | 165 | 11.6 | 12.1 | −3.9 | 31 |
| GLU | O | B | 165 | 10.6 | 12.4 | −4.5 | 29 |
| ILE | N | B | 166 | 12.7 | 12.9 | −3.7 | 28 |
| ILE | CA | B | 166 | 12.7 | 14.2 | −4.3 | 27 |
| ILE | CB | B | 166 | 13.0 | 15.3 | −3.2 | 25 |
| ILE | CG2 | B | 166 | 11.9 | 15.3 | −2.1 | 22 |
| ILE | CG1 | B | 166 | 14.4 | 15.0 | −2.6 | 22 |
| ILE | CD1 | B | 166 | 14.7 | 16.1 | −1.5 | 18 |
| ILE | C | B | 166 | 13.6 | 14.4 | −5.5 | 25 |
| ILE | O | B | 166 | 13.8 | 15.6 | −5.9 | 24 |
| ALA | N | B | 167 | 14.1 | 13.3 | −6.0 | 25 |
| ALA | CA | B | 167 | 14.9 | 13.4 | −7.2 | 28 |
| ALA | CB | B | 167 | 15.7 | 12.0 | −7.3 | 23 |
| ALA | C | B | 167 | 14.1 | 13.6 | −8.5 | 28 |
| ALA | O | B | 167 | 13.0 | 13.2 | −8.6 | 32 |
| ARG | N | B | 168 | 14.8 | 14.4 | −9.4 | 31 |
| ARG | CA | B | 168 | 14.2 | 14.7 | −10.7 | 29 |
| ARG | CB | B | 168 | 14.4 | 16.2 | −11.1 | 34 |
| ARG | CG | B | 168 | 13.7 | 17.2 | −10.2 | 38 |
| ARG | CD | B | 168 | 12.2 | 16.9 | −10.1 | 40 |
| ARG | NE | B | 168 | 11.6 | 17.9 | −9.3 | 44 |
| ARG | CZ | B | 168 | 11.3 | 19.2 | −9.6 | 45 |
| ARG | NH1 | B | 168 | 11.7 | 19.6 | −10.8 | 44 |
| ARG | NH2 | B | 168 | 10.7 | 20.0 | −8.7 | 43 |
| ARG | C | B | 168 | 14.9 | 13.8 | −11.7 | 27 |
| ARG | O | B | 168 | 16.1 | 13.5 | −11.6 | 23 |
| PRO | N | B | 169 | 14.1 | 13.3 | −12.8 | 28 |
| PRO | CD | B | 169 | 14.8 | 13.0 | −14.0 | 25 |
| PRO | CA | B | 169 | 12.7 | 13.6 | −13.1 | 24 |
| PRO | CB | B | 169 | 12.6 | 12.9 | −14.4 | 25 |
| PRO | CG | B | 169 | 13.8 | 13.4 | −15.1 | 27 |
| PRO | C | B | 169 | 11.8 | 13.0 | −12.1 | 25 |
| PRO | O | B | 169 | 10.7 | 13.5 | −11.8 | 31 |
| ASP | N | B | 170 | 12.2 | 11.9 | −11.4 | 28 |
| ASP | CA | B | 170 | 11.3 | 11.2 | −10.5 | 28 |
| ASP | CB | B | 170 | 10.1 | 10.5 | −11.1 | 33 |
| ASP | CG | B | 170 | 10.5 | 9.5 | −12.2 | 38 |
| ASP | OD1 | B | 170 | 10.0 | 9.6 | −13.3 | 40 |
| ASP | OD2 | B | 170 | 11.4 | 8.6 | −11.9 | 35 |
| ASP | C | B | 170 | 12.1 | 10.3 | −9.5 | 29 |
| ASP | O | B | 170 | 13.3 | 10.1 | −9.7 | 26 |
| ASP | N | B | 171 | 11.5 | 9.7 | −8.6 | 28 |
| ASP | CA | B | 171 | 12.1 | 8.8 | −7.6 | 32 |
| ASP | CB | B | 171 | 11.2 | 8.4 | −6.5 | 38 |
| ASP | CG | B | 171 | 10.0 | 7.5 | −6.9 | 40 |
| ASP | OD1 | B | 171 | 10.0 | 7.1 | −8.1 | 41 |
| ASP | OD2 | B | 171 | 9.1 | 7.2 | −6.1 | 43 |
| ASP | C | B | 171 | 12.9 | 7.6 | −8.2 | 31 |
| ASP | O | B | 171 | 13.5 | 6.8 | −7.4 | 33 |
| SER | N | B | 172 | 12.8 | 7.4 | −9.5 | 30 |
| SER | CA | B | 172 | 13.5 | 6.3 | −10.1 | 31 |
| SER | CB | B | 172 | 12.7 | 5.7 | −11.2 | 32 |
| SER | OG | B | 172 | 12.6 | 6.6 | −12.3 | 39 |
| SER | C | B | 172 | 14.9 | 6.6 | −10.5 | 29 |
| SER | O | B | 172 | 15.7 | 5.7 | −10.9 | 27 |
| LEU | N | B | 173 | 15.3 | 7.9 | −10.4 | 28 |
| LEU | CA | B | 173 | 16.6 | 8.3 | −10.7 | 28 |
| LEU | CB | B | 173 | 16.7 | 9.7 | −11.2 | 25 |
| LEU | CG | B | 173 | 18.1 | 10.1 | −11.7 | 26 |
| LEU | CD1 | B | 173 | 18.4 | 9.2 | −12.9 | 24 |
| LEU | CD2 | B | 173 | 18.2 | 11.6 | −12.1 | 29 |
| LEU | C | B | 173 | 17.4 | 8.1 | −9.4 | 29 |
| LEU | O | B | 173 | 17.3 | 8.9 | −8.5 | 24 |
| GLU | N | B | 174 | 18.2 | 7.0 | −9.4 | 28 |
| GLU | CA | B | 174 | 18.9 | 6.7 | −8.2 | 25 |
| GLU | CB | B | 174 | 19.6 | 5.3 | −8.4 | 27 |
| GLU | CG | B | 174 | 20.4 | 4.8 | −7.1 | 29 |
| GLU | CD | B | 174 | 21.0 | 3.4 | −7.5 | 31 |
| GLU | OE1 | B | 174 | 20.5 | 2.4 | −6.9 | 33 |
| GLU | OE2 | B | 174 | 22.0 | 3.4 | −8.2 | 32 |
| GLU | C | B | 174 | 20.0 | 7.7 | −7.8 | 22 |
| GLU | O | B | 174 | 20.8 | 8.1 | −8.6 | 20 |
| PRO | N | B | 175 | 19.9 | 8.3 | −6.5 | 25 |
| PRO | CD | B | 175 | 18.6 | 8.3 | −5.8 | 26 |
| PRO | CA | B | 175 | 20.8 | 9.3 | −6.0 | 21 |
| PRO | CB | B | 175 | 20.1 | 9.5 | −4.6 | 22 |
| PRO | CG | B | 175 | 18.7 | 9.7 | −5.0 | 26 |
| PRO | C | B | 175 | 22.2 | 8.7 | −5.9 | 24 |
| PRO | O | B | 175 | 22.3 | 7.5 | −5.7 | 28 |
| PHE | N | B | 176 | 23.2 | 9.5 | −6.0 | 22 |
| PHE | CA | B | 176 | 24.6 | 9.1 | −5.9 | 19 |
| PHE | CB | B | 176 | 25.5 | 10.3 | −5.9 | 19 |
| PHE | CG | B | 176 | 27.0 | 9.9 | −5.6 | 21 |
| PHE | CD1 | B | 176 | 27.7 | 9.3 | −6.6 | 24 |
| PHE | CD2 | B | 176 | 27.5 | 10.1 | −4.3 | 22 |
| PHE | CE1 | B | 176 | 29.0 | 8.9 | −6.3 | 22 |
| PHE | CE2 | B | 176 | 28.8 | 9.6 | −4.0 | 22 |
| PHE | CZ | B | 176 | 29.6 | 9.1 | −5.0 | 20 |
| PHE | C | B | 176 | 24.9 | 8.2 | −4.7 | 21 |
| PHE | O | B | 176 | 25.6 | 7.2 | −4.8 | 18 |
| PHE | N | B | 177 | 24.4 | 8.7 | −3.5 | 20 |
| PHE | CA | B | 177 | 24.8 | 7.9 | −2.3 | 21 |
| PHE | CB | B | 177 | 24.5 | 8.8 | −1.1 | 21 |
| PHE | CG | B | 177 | 25.1 | 8.2 | 0.2 | 22 |
| PHE | CD1 | B | 177 | 26.4 | 8.5 | 0.5 | 18 |
| PHE | CD2 | B | 177 | 24.4 | 7.3 | 1.0 | 22 |
| PHE | CE1 | B | 177 | 27.1 | 8.0 | 1.6 | 19 |
| PHE | CE2 | B | 177 | 25.0 | 6.8 | 2.1 | 22 |
| PHE | CZ | B | 177 | 26.3 | 7.1 | 2.5 | 18 |
| PHE | C | B | 177 | 24.2 | 6.6 | −2.3 | 24 |
| PHE | O | B | 177 | 24.7 | 5.6 | −1.7 | 28 |
| ASP | N | B | 178 | 23.0 | 6.4 | −2.9 | 25 |
| ASP | CA | B | 178 | 22.3 | 5.1 | −3.0 | 27 |
| ASP | CB | B | 178 | 20.9 | 5.2 | −3.5 | 27 |
| ASP | CG | B | 178 | 20.0 | 6.0 | −2.5 | 33 |
| ASP | OD1 | B | 178 | 19.0 | 5.4 | −2.0 | 31 |
| ASP | OD2 | B | 178 | 20.2 | 7.2 | −2.3 | 34 |
| ASP | C | B | 178 | 23.1 | 4.2 | −3.9 | 26 |
| ASP | O | B | 178 | 23.3 | 3.0 | −3.6 | 23 |
| SER | N | B | 179 | 23.7 | 4.8 | −4.9 | 24 |
| SER | CA | B | 179 | 24.5 | 4.0 | −5.9 | 23 |
| SER | CB | B | 179 | 24.8 | 4.7 | −7.1 | 19 |
| SER | OG | B | 179 | 23.7 | 5.2 | −7.8 | 24 |
| SER | C | B | 179 | 25.7 | 3.5 | −5.1 | 25 |
| SER | O | B | 179 | 26.2 | 2.4 | −5.3 | 24 |
| LEU | N | B | 180 | 26.3 | 4.4 | −4.4 | 24 |
| LEU | CA | B | 180 | 27.5 | 4.2 | −3.6 | 25 |
| LEU | CB | B | 180 | 28.0 | 5.4 | −2.9 | 20 |
| LEU | CG | B | 180 | 29.2 | 5.1 | −2.0 | 23 |
| LEU | CD1 | B | 180 | 30.4 | 4.6 | −2.9 | 19 |
| LEU | CD2 | B | 180 | 29.7 | 6.4 | −1.3 | 21 |
| LEU | C | B | 180 | 27.3 | 3.0 | −2.7 | 24 |
| LEU | O | B | 180 | 28.2 | 2.1 | −2.6 | 23 |
| VAL | N | B | 181 | 26.2 | 3.0 | −1.9 | 23 |
| VAL | CA | B | 181 | 26.0 | 1.9 | −1.0 | 24 |
| VAL | CB | B | 181 | 24.9 | 2.4 | 0.1 | 22 |
| VAL | CG1 | B | 181 | 24.6 | 1.2 | 1.0 | 22 |
| VAL | CG2 | B | 181 | 25.5 | 3.6 | 0.9 | 22 |
| VAL | C | B | 181 | 25.5 | 0.6 | −1.6 | 26 |
| VAL | O | B | 181 | 25.8 | −0.4 | −1.1 | 28 |
| LYS | N | B | 182 | 24.9 | 0.7 | −2.8 | 25 |
| LYS | CA | B | 182 | 24.4 | −0.6 | −3.4 | 29 |
| LYS | CB | B | 182 | 23.3 | −0.3 | −4.4 | 29 |
| LYS | CG | B | 182 | 22.8 | −1.6 | −5.1 | 32 |
| LYS | CD | B | 182 | 21.7 | −1.3 | −6.1 | 37 |
| LYS | CE | B | 182 | 22.1 | −0.4 | −7.3 | 37 |
| LYS | NZ | B | 182 | 21.0 | −0.2 | −8.3 | 35 |
| LYS | C | B | 182 | 25.6 | −1.3 | −4.1 | 27 |
| LYS | O | B | 182 | 25.5 | −2.5 | −4.2 | 33 |
| GLN | N | B | 183 | 26.6 | −0.6 | −4.5 | 25 |
| GLN | CA | B | 183 | 27.7 | −1.2 | −5.2 | 22 |
| GLN | CB | B | 183 | 28.1 | −0.3 | −6.4 | 23 |
| GLN | CG | B | 183 | 27.0 | 0.0 | −7.4 | 22 |
| GLN | CD | B | 183 | 27.5 | 0.9 | −8.5 | 21 |
| GLN | OE1 | B | 183 | 28.7 | 1.0 | −8.7 | 26 |
| GLN | NE2 | B | 183 | 26.6 | 1.6 | −9.1 | 27 |
| GLN | C | B | 183 | 28.9 | −1.5 | −4.3 | 21 |
| GLN | O | B | 183 | 29.8 | −2.3 | −4.7 | 19 |
| THR | N | B | 184 | 29.0 | −0.9 | −3.2 | 20 |
| THR | CA | B | 184 | 30.2 | −1.1 | −2.3 | 23 |
| THR | CB | B | 184 | 31.1 | 0.1 | −2.3 | 26 |
| THR | OG1 | B | 184 | 30.5 | 1.2 | −1.6 | 24 |
| THR | CG2 | B | 184 | 31.5 | 0.6 | −3.7 | 27 |
| THR | C | B | 184 | 29.9 | −1.5 | −0.9 | 25 |
| THR | O | B | 184 | 28.7 | −1.7 | −0.6 | 26 |
| HIS | N | B | 185 | 30.9 | −1.6 | −0.1 | 22 |
| HIS | CA | B | 185 | 30.8 | −1.9 | 1.3 | 30 |
| HIS | CB | B | 185 | 31.8 | −2.9 | 1.8 | 33 |
| HIS | CG | B | 185 | 31.9 | −4.2 | 1.1 | 38 |
| HIS | CD2 | B | 185 | 30.9 | −5.2 | 1.0 | 41 |
| HIS | ND1 | B | 185 | 32.9 | −4.6 | 0.3 | 39 |
| HIS | CE1 | B | 185 | 32.6 | −5.8 | −0.2 | 41 |
| HIS | NE2 | B | 185 | 31.4 | −6.1 | 0.2 | 43 |
| HIS | C | B | 185 | 30.7 | −0.7 | 2.2 | 25 |
| HIS | O | B | 185 | 30.7 | −0.8 | 3.4 | 25 |
| VAL | N | B | 186 | 30.6 | 0.5 | 1.6 | 24 |
| VAL | CA | B | 186 | 30.5 | 1.8 | 2.3 | 22 |
| VAL | CB | B | 186 | 30.6 | 3.0 | 1.4 | 17 |
| VAL | CG1 | B | 186 | 30.5 | 4.3 | 2.2 | 19 |
| VAL | CG2 | B | 186 | 32.0 | 3.0 | 0.7 | 18 |
| VAL | C | B | 186 | 29.2 | 1.8 | 3.1 | 24 |
| VAL | O | B | 186 | 28.1 | 1.7 | 2.5 | 23 |
| PRO | N | B | 187 | 29.3 | 1.9 | 4.4 | 25 |
| PRO | CD | B | 187 | 30.5 | 1.7 | 5.2 | 23 |
| PRO | CA | B | 187 | 28.1 | 2.0 | 5.3 | 25 |
| PRO | CB | B | 187 | 28.8 | 2.3 | 6.7 | 25 |
| PRO | CG | B | 187 | 29.9 | 1.3 | 6.6 | 23 |
| PRO | C | B | 187 | 27.1 | 3.0 | 4.9 | 25 |
| PRO | O | B | 187 | 27.5 | 4.1 | 4.4 | 23 |
| ASN | N | B | 188 | 25.8 | 2.7 | 5.0 | 23 |
| ASN | CA | B | 188 | 24.8 | 3.7 | 4.6 | 22 |
| ASN | CB | B | 188 | 23.5 | 3.0 | 4.4 | 22 |
| ASN | CG | B | 188 | 22.4 | 3.9 | 3.9 | 25 |
| ASN | OD1 | B | 188 | 22.7 | 5.0 | 3.4 | 28 |
| ASN | ND2 | B | 188 | 21.1 | 3.5 | 4.0 | 23 |
| ASN | C | B | 188 | 24.7 | 4.8 | 5.7 | 23 |
| ASN | O | B | 188 | 23.8 | 4.8 | 6.4 | 25 |
| LEU | N | B | 189 | 25.8 | 5.6 | 5.7 | 21 |
| LEU | CA | B | 189 | 25.9 | 6.7 | 6.7 | 22 |
| LEU | CB | B | 189 | 26.0 | 6.1 | 8.1 | 29 |
| LEU | CG | B | 189 | 26.1 | 7.0 | 9.3 | 34 |
| LEU | CD1 | B | 189 | 26.1 | 6.3 | 10.6 | 33 |
| LEU | CD2 | B | 189 | 27.4 | 8.0 | 9.2 | 38 |
| LEU | C | B | 189 | 27.0 | 7.7 | 6.3 | 21 |
| LEU | O | B | 189 | 28.0 | 7.3 | 5.8 | 20 |
| PHE | N | B | 190 | 26.7 | 8.9 | 6.5 | 19 |
| PHE | CA | B | 190 | 27.7 | 10.0 | 6.3 | 20 |
| PHE | CB | B | 190 | 27.7 | 10.6 | 4.9 | 19 |
| PHE | CG | B | 190 | 26.4 | 11.3 | 4.6 | 21 |
| PHE | CD1 | B | 190 | 25.3 | 10.7 | 4.0 | 19 |
| PHE | CD2 | B | 190 | 26.3 | 12.7 | 4.8 | 21 |
| PHE | CE1 | B | 190 | 24.2 | 11.4 | 3.7 | 21 |
| PHE | CE2 | B | 190 | 25.2 | 13.4 | 4.5 | 18 |
| PHE | CZ | B | 190 | 24.1 | 12.8 | 4.0 | 18 |
| PHE | C | B | 190 | 27.6 | 11.0 | 7.4 | 19 |
| PHE | O | B | 190 | 26.5 | 11.2 | 8.0 | 20 |
| SER | N | B | 191 | 28.7 | 11.7 | 7.7 | 23 |
| SER | CA | B | 191 | 28.6 | 12.7 | 8.7 | 23 |
| SER | CB | B | 191 | 29.3 | 12.2 | 10.0 | 19 |
| SER | OG | B | 191 | 30.6 | 11.9 | 9.8 | 22 |
| SER | C | B | 191 | 29.3 | 14.0 | 8.2 | 22 |
| SER | O | B | 191 | 30.2 | 14.0 | 7.4 | 23 |
| LEU | N | B | 192 | 28.8 | 15.2 | 8.7 | 21 |
| LEU | CA | B | 192 | 29.3 | 16.5 | 8.3 | 24 |
| LEU | CB | B | 192 | 28.2 | 17.2 | 7.4 | 22 |
| LEU | CG | B | 192 | 27.9 | 16.6 | 6.1 | 23 |
| LEU | CD1 | B | 192 | 26.8 | 17.3 | 5.4 | 16 |
| LEU | CD2 | B | 192 | 29.2 | 16.5 | 5.2 | 19 |
| LEU | C | B | 192 | 29.7 | 17.4 | 9.4 | 22 |
| LEU | O | B | 192 | 29.0 | 17.6 | 10.4 | 20 |
| GLN | N | B | 193 | 31.0 | 17.9 | 9.3 | 24 |
| GLN | CA | B | 193 | 31.5 | 18.9 | 10.2 | 25 |
| GLN | CB | B | 193 | 32.8 | 18.5 | 10.8 | 27 |
| GLN | CG | B | 193 | 33.3 | 19.7 | 11.7 | 29 |
| GLN | CD | B | 193 | 34.7 | 19.3 | 12.3 | 30 |
| GLN | OE1 | B | 193 | 35.7 | 19.4 | 11.7 | 30 |
| GLN | NE2 | B | 193 | 34.7 | 18.9 | 13.6 | 30 |
| GLN | C | B | 193 | 31.8 | 20.2 | 9.3 | 26 |
| GLN | O | B | 193 | 32.7 | 20.2 | 8.6 | 21 |
| LEU | N | B | 194 | 30.9 | 21.2 | 9.4 | 24 |
| LEU | CA | B | 194 | 31.1 | 22.4 | 8.7 | 23 |
| LEU | CB | B | 194 | 29.7 | 22.9 | 8.1 | 26 |
| LEU | CG | B | 194 | 29.0 | 21.9 | 7.3 | 24 |
| LEU | CD1 | B | 194 | 27.6 | 22.5 | 6.8 | 24 |
| LEU | CD2 | B | 194 | 29.8 | 21.4 | 6.1 | 20 |
| LEU | C | B | 194 | 31.7 | 23.4 | 9.6 | 24 |
| LEU | O | B | 194 | 31.2 | 23.6 | 10.7 | 23 |
| CYS | N | B | 195 | 32.9 | 23.9 | 9.3 | 25 |
| CYS | CA | B | 195 | 33.5 | 24.8 | 10.2 | 33 |
| CYS | C | B | 195 | 33.4 | 26.3 | 10.0 | 39 |
| CYS | O | B | 195 | 33.3 | 27.1 | 10.9 | 47 |
| CYS | CB | B | 195 | 35.0 | 24.4 | 10.4 | 31 |
| CYS | SG | B | 195 | 35.2 | 22.7 | 11.0 | 32 |
| GLY | N | B | 196 | 33.3 | 26.7 | 8.7 | 44 |
| GLY | CA | B | 196 | 33.1 | 28.1 | 8.4 | 54 |
| GLY | C | B | 196 | 33.9 | 29.2 | 9.2 | 58 |
| GLY | O | B | 196 | 33.6 | 30.4 | 9.1 | 62 |
| ALA | N | B | 197 | 35.0 | 28.7 | 9.9 | 62 |
| ALA | CA | B | 197 | 35.8 | 29.7 | 10.6 | 64 |
| ALA | CB | B | 197 | 37.0 | 28.9 | 11.3 | 63 |
| ALA | C | B | 197 | 36.4 | 30.8 | 9.7 | 65 |
| ALA | O | B | 197 | 36.3 | 31.9 | 10.0 | 66 |
| SER | N | B | 209 | 39.0 | 30.5 | 3.6 | 49 |
| SER | CA | B | 209 | 37.8 | 31.0 | 4.3 | 53 |
| SER | CB | B | 209 | 36.8 | 31.6 | 3.2 | 55 |
| SER | OG | B | 209 | 37.4 | 32.6 | 2.5 | 61 |
| SER | C | B | 209 | 37.1 | 30.0 | 5.2 | 49 |
| SER | O | B | 209 | 37.1 | 30.2 | 6.4 | 55 |
| VAL | N | B | 210 | 36.5 | 29.0 | 4.6 | 44 |
| VAL | CA | B | 210 | 35.8 | 28.0 | 5.3 | 38 |
| VAL | CB | B | 210 | 34.3 | 28.2 | 5.2 | 36 |
| VAL | CG1 | B | 210 | 33.8 | 29.6 | 5.7 | 39 |
| VAL | CG2 | B | 210 | 33.7 | 27.9 | 3.8 | 36 |
| VAL | C | B | 210 | 36.2 | 26.6 | 4.9 | 34 |
| VAL | O | B | 210 | 36.5 | 26.4 | 3.7 | 39 |
| GLY | N | B | 211 | 36.2 | 25.7 | 5.8 | 31 |
| GLY | CA | B | 211 | 36.5 | 24.3 | 5.5 | 29 |
| GLY | C | B | 211 | 35.7 | 23.4 | 6.4 | 25 |
| GLY | O | B | 211 | 34.8 | 23.8 | 7.1 | 25 |
| GLY | N | B | 212 | 35.9 | 22.1 | 6.2 | 22 |
| GLY | CA | B | 212 | 35.2 | 21.1 | 7.0 | 22 |
| GLY | C | B | 212 | 35.5 | 19.7 | 6.6 | 23 |
| GLY | O | B | 212 | 36.4 | 19.4 | 5.8 | 21 |
| SER | N | B | 213 | 34.7 | 18.7 | 7.1 | 20 |
| SER | CA | B | 213 | 34.8 | 17.3 | 6.8 | 20 |
| SER | CB | B | 213 | 35.5 | 16.6 | 8.0 | 22 |
| SER | OG | B | 213 | 36.8 | 17.1 | 8.3 | 26 |
| SER | C | B | 213 | 33.5 | 16.6 | 6.5 | 21 |
| SER | O | B | 213 | 32.5 | 16.7 | 7.2 | 24 |
| MET | N | B | 214 | 33.6 | 15.8 | 5.4 | 20 |
| MET | CA | B | 214 | 32.4 | 15.0 | 5.1 | 23 |
| MET | CB | B | 214 | 31.9 | 15.2 | 3.7 | 23 |
| MET | CG | B | 214 | 30.8 | 14.3 | 3.3 | 25 |
| MET | SD | B | 214 | 30.0 | 14.5 | 1.7 | 27 |
| MET | CE | B | 214 | 31.5 | 14.3 | 0.6 | 27 |
| MET | C | B | 214 | 33.0 | 13.6 | 5.2 | 21 |
| MET | O | B | 214 | 33.8 | 13.1 | 4.4 | 22 |
| ILE | N | B | 215 | 32.6 | 12.9 | 6.2 | 19 |
| ILE | CA | B | 215 | 33.1 | 11.5 | 6.5 | 19 |
| ILE | CB | B | 215 | 33.2 | 11.2 | 8.0 | 20 |
| ILE | CG2 | B | 215 | 33.8 | 9.8 | 8.1 | 13 |
| ILE | CG1 | B | 215 | 34.1 | 12.2 | 8.7 | 20 |
| ILE | CD1 | B | 215 | 35.6 | 12.3 | 8.1 | 20 |
| ILE | C | B | 215 | 32.1 | 10.6 | 5.8 | 22 |
| ILE | O | B | 215 | 30.9 | 10.5 | 6.3 | 21 |
| ILE | N | B | 216 | 32.5 | 9.9 | 4.7 | 22 |
| ILE | CA | B | 216 | 31.7 | 9.0 | 4.0 | 28 |
| ILE | CB | B | 216 | 32.0 | 9.0 | 2.5 | 30 |
| ILE | CG2 | B | 216 | 31.1 | 8.0 | 1.8 | 28 |
| ILE | CG1 | B | 216 | 32.0 | 10.3 | 1.9 | 30 |
| ILE | CD1 | B | 216 | 30.7 | 11.0 | 1.9 | 39 |
| ILE | C | B | 216 | 31.7 | 7.6 | 4.6 | 26 |
| ILE | O | B | 216 | 32.8 | 7.0 | 4.7 | 29 |
| GLY | N | B | 217 | 30.5 | 7.1 | 5.0 | 27 |
| GLY | CA | B | 217 | 30.5 | 5.8 | 5.6 | 23 |
| GLY | C | B | 217 | 30.8 | 5.7 | 7.1 | 26 |
| GLY | O | B | 217 | 30.9 | 4.5 | 7.6 | 30 |
| GLY | N | B | 218 | 31.0 | 6.8 | 7.8 | 27 |
| GLY | CA | B | 218 | 31.3 | 6.7 | 9.2 | 28 |
| GLY | C | B | 218 | 31.3 | 7.9 | 10.0 | 32 |
| GLY | O | B | 218 | 30.9 | 9.0 | 9.6 | 30 |
| ILE | N | B | 219 | 31.7 | 7.7 | 11.3 | 30 |
| ILE | CA | B | 219 | 31.7 | 8.8 | 12.2 | 28 |
| ILE | CB | B | 219 | 30.8 | 8.5 | 13.4 | 26 |
| ILE | CG2 | B | 219 | 30.9 | 9.6 | 14.5 | 26 |
| ILE | CG1 | B | 219 | 29.4 | 8.3 | 12.9 | 24 |
| ILE | CD1 | B | 219 | 28.4 | 8.0 | 14.0 | 21 |
| ILE | C | B | 219 | 33.2 | 9.0 | 12.8 | 30 |
| ILE | O | B | 219 | 33.7 | 8.0 | 13.3 | 35 |
| ASP | N | B | 220 | 33.7 | 10.2 | 12.7 | 29 |
| ASP | CA | B | 220 | 35.1 | 10.4 | 13.2 | 27 |
| ASP | CB | B | 220 | 36.0 | 11.2 | 12.2 | 26 |
| ASP | CG | B | 220 | 37.4 | 11.3 | 12.7 | 32 |
| ASP | OD1 | B | 220 | 38.3 | 11.1 | 11.9 | 32 |
| ASP | OD2 | B | 220 | 37.6 | 11.7 | 13.8 | 34 |
| ASP | C | B | 220 | 35.0 | 11.1 | 14.5 | 31 |
| ASP | O | B | 220 | 34.5 | 12.2 | 14.6 | 24 |
| HIS | N | B | 221 | 35.4 | 10.4 | 15.6 | 28 |
| HIS | CA | B | 221 | 35.2 | 10.9 | 16.9 | 30 |
| HIS | CB | B | 221 | 35.4 | 9.7 | 17.9 | 35 |
| HIS | CG | B | 221 | 34.3 | 8.7 | 17.7 | 38 |
| HIS | CD2 | B | 221 | 34.4 | 7.4 | 17.2 | 40 |
| HIS | ND1 | B | 221 | 33.0 | 8.9 | 18.0 | 41 |
| HIS | CE1 | B | 221 | 32.3 | 7.8 | 17.7 | 40 |
| HIS | NE2 | B | 221 | 33.1 | 6.9 | 17.2 | 40 |
| HIS | C | B | 221 | 36.1 | 12.1 | 17.3 | 31 |
| HIS | O | B | 221 | 35.9 | 12.6 | 18.4 | 34 |
| SER | N | B | 222 | 37.0 | 12.6 | 16.5 | 31 |
| SER | CA | B | 222 | 37.8 | 13.7 | 16.8 | 30 |
| SER | CB | B | 222 | 39.2 | 13.6 | 16.2 | 27 |
| SER | OG | B | 222 | 39.1 | 13.7 | 14.8 | 28 |
| SER | C | B | 222 | 37.1 | 15.0 | 16.4 | 29 |
| SER | O | B | 222 | 37.6 | 16.1 | 16.6 | 30 |
| LEU | N | B | 223 | 36.0 | 14.8 | 15.7 | 27 |
| LEU | CA | B | 223 | 35.2 | 16.0 | 15.2 | 29 |
| LEU | CB | B | 223 | 34.6 | 15.6 | 13.8 | 25 |
| LEU | CG | B | 223 | 35.6 | 15.1 | 12.8 | 26 |
| LEU | CD1 | B | 223 | 34.8 | 14.7 | 11.5 | 22 |
| LEU | CD2 | B | 223 | 36.6 | 16.2 | 12.5 | 23 |
| LEU | C | B | 223 | 34.2 | 16.6 | 16.1 | 27 |
| LEU | O | B | 223 | 33.7 | 17.6 | 15.9 | 27 |
| TYR | N | B | 224 | 34.0 | 15.9 | 17.3 | 24 |
| TYR | CA | B | 224 | 33.1 | 16.5 | 18.3 | 24 |
| TYR | CB | B | 224 | 31.7 | 15.9 | 18.1 | 26 |
| TYR | CG | B | 224 | 31.6 | 14.4 | 18.3 | 27 |
| TYR | CD1 | B | 224 | 32.0 | 13.5 | 17.3 | 28 |
| TYR | CE1 | B | 224 | 31.8 | 12.1 | 17.5 | 29 |
| TYR | CD2 | B | 224 | 31.0 | 13.9 | 19.5 | 26 |
| TYR | CE2 | B | 224 | 30.9 | 12.5 | 19.7 | 28 |
| TYR | CZ | B | 224 | 31.3 | 11.7 | 18.7 | 30 |
| TYR | OH | B | 224 | 31.1 | 10.3 | 18.9 | 31 |
| TYR | C | B | 224 | 33.5 | 16.2 | 19.7 | 27 |
| TYR | O | B | 224 | 34.5 | 15.4 | 19.9 | 24 |
| THR | N | B | 225 | 32.9 | 16.8 | 20.6 | 27 |
| THR | CA | B | 225 | 33.1 | 16.6 | 22.0 | 28 |
| THR | CB | B | 225 | 33.7 | 17.9 | 22.7 | 29 |
| THR | OG1 | B | 225 | 32.8 | 19.0 | 22.6 | 27 |
| THR | CG2 | B | 225 | 35.1 | 18.3 | 22.1 | 26 |
| THR | C | B | 225 | 31.8 | 16.3 | 22.6 | 28 |
| THR | O | B | 225 | 30.8 | 16.7 | 22.1 | 29 |
| GLY | N | B | 226 | 31.8 | 15.7 | 23.8 | 28 |
| GLY | CA | B | 226 | 30.5 | 15.3 | 24.4 | 25 |
| GLY | C | B | 226 | 29.8 | 14.2 | 23.8 | 28 |
| GLY | O | B | 226 | 30.3 | 13.4 | 23.1 | 28 |
| SER | N | B | 227 | 28.4 | 14.2 | 24.0 | 29 |
| SER | CA | B | 227 | 27.6 | 13.1 | 23.5 | 32 |
| SER | CB | B | 227 | 26.7 | 12.5 | 24.6 | 36 |
| SER | OG | B | 227 | 27.5 | 12.0 | 25.6 | 42 |
| SER | C | B | 227 | 26.8 | 13.3 | 22.2 | 29 |
| SER | O | B | 227 | 26.4 | 14.5 | 22.0 | 22 |
| LEU | N | B | 228 | 26.6 | 12.3 | 21.4 | 28 |
| LEU | CA | B | 228 | 25.8 | 12.4 | 20.2 | 28 |
| LEU | CB | B | 228 | 26.1 | 11.3 | 19.2 | 31 |
| LEU | CG | B | 228 | 27.5 | 11.4 | 18.5 | 34 |
| LEU | CD1 | B | 228 | 27.8 | 10.2 | 17.6 | 33 |
| LEU | CD2 | B | 228 | 27.5 | 12.7 | 17.7 | 34 |
| LEU | C | B | 228 | 24.3 | 12.2 | 20.6 | 27 |
| LEU | O | B | 228 | 24.0 | 11.2 | 21.2 | 30 |
| TRP | N | B | 229 | 23.5 | 13.1 | 20.2 | 24 |
| TRP | CA | B | 229 | 22.0 | 13.0 | 20.4 | 27 |
| TRP | CB | B | 229 | 21.4 | 14.1 | 21.2 | 23 |
| TRP | CG | B | 229 | 21.8 | 14.1 | 22.6 | 24 |
| TRP | CD2 | B | 229 | 21.0 | 13.6 | 23.7 | 24 |
| TRP | CE2 | B | 229 | 21.8 | 13.7 | 24.9 | 24 |
| TRP | CE3 | B | 229 | 19.7 | 13.1 | 23.8 | 25 |
| TRP | CD1 | B | 229 | 23.0 | 14.5 | 23.2 | 23 |
| TRP | NE1 | B | 229 | 23.0 | 14.3 | 24.5 | 26 |
| TRP | CZ2 | B | 229 | 21.3 | 13.4 | 26.2 | 27 |
| TRP | CZ3 | B | 229 | 19.2 | 12.7 | 25.1 | 28 |
| TRP | CH2 | B | 229 | 20.0 | 12.8 | 26.2 | 27 |
| TRP | C | B | 229 | 21.3 | 12.8 | 19.0 | 27 |
| TRP | O | B | 229 | 21.6 | 13.6 | 18.1 | 27 |
| TYR | N | B | 230 | 20.5 | 11.8 | 19.0 | 26 |
| TYR | CA | B | 230 | 19.7 | 11.5 | 17.8 | 27 |
| TYR | CB | B | 230 | 19.9 | 10.0 | 17.4 | 27 |
| TYR | CG | B | 230 | 21.3 | 9.6 | 17.1 | 24 |
| TYR | CD1 | B | 230 | 22.1 | 9.2 | 18.1 | 23 |
| TYR | CE1 | B | 230 | 23.5 | 8.9 | 17.8 | 24 |
| TYR | CD2 | B | 230 | 21.8 | 9.7 | 15.8 | 20 |
| TYR | CE2 | B | 230 | 23.1 | 9.3 | 15.5 | 17 |
| TYR | CZ | B | 230 | 23.9 | 8.9 | 16.5 | 22 |
| TYR | OH | B | 230 | 25.2 | 8.6 | 16.2 | 27 |
| TYR | C | B | 230 | 18.3 | 11.9 | 17.7 | 26 |
| TYR | O | B | 230 | 17.5 | 11.7 | 18.7 | 26 |
| THR | N | B | 231 | 17.9 | 12.5 | 16.6 | 26 |
| THR | CA | B | 231 | 16.5 | 12.9 | 16.4 | 23 |
| THR | CB | B | 231 | 16.4 | 14.4 | 16.1 | 18 |
| THR | OG1 | B | 231 | 15.0 | 14.8 | 16.0 | 23 |
| THR | CG2 | B | 231 | 17.0 | 14.8 | 14.8 | 20 |
| THR | C | B | 231 | 16.0 | 12.0 | 15.2 | 26 |
| THR | O | B | 231 | 16.8 | 11.8 | 14.2 | 26 |
| PRO | N | B | 232 | 14.8 | 11.5 | 15.2 | 24 |
| PRO | CD | B | 232 | 14.0 | 11.3 | 16.5 | 25 |
| PRO | CA | B | 232 | 14.2 | 10.7 | 14.2 | 28 |
| PRO | CB | B | 232 | 12.9 | 10.4 | 14.7 | 27 |
| PRO | CG | B | 232 | 13.2 | 10.0 | 16.1 | 29 |
| PRO | C | B | 232 | 14.1 | 11.4 | 12.8 | 28 |
| PRO | O | B | 232 | 13.7 | 12.5 | 12.7 | 30 |
| ILE | N | B | 233 | 14.5 | 10.7 | 11.7 | 28 |
| ILE | CA | B | 233 | 14.3 | 11.3 | 10.4 | 28 |
| ILE | CB | B | 233 | 15.1 | 10.6 | 9.3 | 27 |
| ILE | CG2 | B | 233 | 14.6 | 11.0 | 7.9 | 27 |
| ILE | CG1 | B | 233 | 16.6 | 10.9 | 9.5 | 21 |
| ILE | CD1 | B | 233 | 17.5 | 10.3 | 8.5 | 14 |
| ILE | C | B | 233 | 12.8 | 10.9 | 10.2 | 30 |
| ILE | O | B | 233 | 12.4 | 9.8 | 10.1 | 29 |
| ARG | N | B | 234 | 11.9 | 11.9 | 10.1 | 33 |
| ARG | CA | B | 234 | 10.5 | 11.7 | 10.0 | 38 |
| ARG | CB | B | 234 | 9.7 | 13.0 | 10.2 | 35 |
| ARG | CG | B | 234 | 8.2 | 12.9 | 10.2 | 34 |
| ARG | CD | B | 234 | 7.5 | 14.2 | 10.4 | 36 |
| ARG | NE | B | 234 | 6.1 | 14.1 | 10.4 | 39 |
| ARG | CZ | B | 234 | 5.2 | 15.1 | 9.9 | 37 |
| ARG | NH1 | B | 234 | 5.7 | 16.2 | 9.4 | 37 |
| ARG | NH2 | B | 234 | 3.9 | 14.9 | 10.1 | 37 |
| ARG | C | B | 234 | 10.0 | 11.0 | 8.8 | 39 |
| ARG | O | B | 234 | 9.2 | 10.1 | 8.8 | 41 |
| ARG | N | B | 235 | 10.6 | 11.4 | 7.7 | 40 |
| ARG | CA | B | 235 | 10.3 | 10.9 | 6.3 | 39 |
| ARG | CB | B | 235 | 9.1 | 11.8 | 5.7 | 41 |
| ARG | CG | B | 235 | 8.8 | 11.4 | 4.3 | 38 |
| ARG | CD | B | 235 | 7.7 | 12.3 | 3.8 | 42 |
| ARG | NE | B | 235 | 7.4 | 12.0 | 2.4 | 44 |
| ARG | CZ | B | 235 | 6.8 | 12.8 | 1.5 | 47 |
| ARG | NH1 | B | 235 | 6.4 | 14.0 | 2.0 | 47 |
| ARG | NH2 | B | 235 | 6.5 | 12.5 | 0.3 | 53 |
| ARG | C | B | 235 | 11.5 | 10.9 | 5.4 | 36 |
| ARG | O | B | 235 | 12.2 | 11.9 | 5.4 | 36 |
| GLU | N | B | 236 | 11.7 | 9.8 | 4.7 | 32 |
| GLU | CA | B | 236 | 12.8 | 9.7 | 3.8 | 32 |
| GLU | CB | B | 236 | 13.3 | 8.3 | 3.6 | 36 |
| GLU | CG | B | 236 | 13.8 | 7.7 | 4.9 | 43 |
| GLU | CD | B | 236 | 14.2 | 6.2 | 4.7 | 46 |
| GLU | OE1 | B | 236 | 13.8 | 5.4 | 5.5 | 51 |
| GLU | OE2 | B | 236 | 14.9 | 6.0 | 3.8 | 52 |
| GLU | C | B | 236 | 12.6 | 10.4 | 2.4 | 32 |
| GLU | O | B | 236 | 12.2 | 9.8 | 1.4 | 32 |
| TRP | N | B | 237 | 12.8 | 11.7 | 2.4 | 30 |
| TRP | CA | B | 237 | 12.6 | 12.6 | 1.2 | 28 |
| TRP | CB | B | 237 | 11.2 | 13.1 | 1.0 | 27 |
| TRP | CG | B | 237 | 10.6 | 14.0 | 2.1 | 29 |
| TRP | CD2 | B | 237 | 9.7 | 15.0 | 2.0 | 27 |
| TRP | CE2 | B | 237 | 9.4 | 15.5 | 3.3 | 28 |
| TRP | CE3 | B | 237 | 9.0 | 15.6 | 0.9 | 31 |
| TRP | CD1 | B | 237 | 10.9 | 13.9 | 3.5 | 28 |
| TRP | NE1 | B | 237 | 10.1 | 14.8 | 4.2 | 28 |
| TRP | CZ2 | B | 237 | 8.4 | 16.6 | 3.5 | 29 |
| TRP | CZ3 | B | 237 | 8.1 | 16.6 | 1.1 | 32 |
| TRP | CH2 | B | 237 | 7.8 | 17.1 | 2.4 | 30 |
| TRP | C | B | 237 | 13.7 | 13.6 | 1.5 | 27 |
| TRP | O | B | 237 | 14.8 | 13.4 | 1.0 | 28 |
| TYR | N | B | 238 | 13.4 | 14.7 | 2.2 | 27 |
| TYR | CA | B | 238 | 14.5 | 15.7 | 2.6 | 24 |
| TYR | CB | B | 238 | 13.9 | 17.1 | 2.9 | 25 |
| TYR | CG | B | 238 | 13.3 | 17.8 | 1.7 | 23 |
| TYR | CD1 | B | 238 | 14.1 | 18.5 | 0.8 | 20 |
| TYR | CE1 | B | 238 | 13.5 | 19.2 | −0.2 | 22 |
| TYR | CD2 | B | 238 | 11.9 | 17.8 | 1.6 | 21 |
| TYR | CE2 | B | 238 | 11.3 | 18.5 | 0.5 | 20 |
| TYR | CZ | B | 238 | 12.1 | 19.2 | −0.3 | 22 |
| TYR | OH | B | 238 | 11.5 | 19.9 | −1.4 | 26 |
| TYR | C | B | 238 | 14.9 | 15.0 | 3.9 | 24 |
| TYR | O | B | 238 | 14.2 | 14.0 | 4.3 | 23 |
| TYR | N | B | 239 | 15.9 | 15.5 | 4.5 | 28 |
| TYR | CA | B | 239 | 16.2 | 15.0 | 5.9 | 27 |
| TYR | CB | B | 239 | 17.7 | 15.1 | 6.3 | 25 |
| TYR | CG | B | 239 | 18.5 | 14.2 | 5.4 | 22 |
| TYR | CD1 | B | 239 | 19.2 | 14.6 | 4.3 | 19 |
| TYR | CE1 | B | 239 | 20.0 | 13.8 | 3.5 | 18 |
| TYR | CD2 | B | 239 | 18.7 | 12.8 | 5.8 | 17 |
| TYR | CE2 | B | 239 | 19.4 | 12.0 | 5.0 | 14 |
| TYR | CZ | B | 239 | 20.1 | 12.4 | 3.9 | 19 |
| TYR | OH | B | 239 | 20.8 | 11.5 | 3.2 | 16 |
| TYR | C | B | 239 | 15.3 | 15.8 | 6.8 | 29 |
| TYR | O | B | 239 | 15.6 | 16.9 | 7.3 | 28 |
| GLU | N | B | 240 | 14.1 | 15.3 | 7.0 | 30 |
| GLU | CA | B | 240 | 13.1 | 15.9 | 7.8 | 29 |
| GLU | CB | B | 240 | 11.6 | 15.7 | 7.3 | 28 |
| GLU | CG | B | 240 | 10.6 | 16.4 | 8.2 | 31 |
| GLU | CD | B | 240 | 9.2 | 16.1 | 7.6 | 32 |
| GLU | OE1 | B | 240 | 9.1 | 15.3 | 6.7 | 36 |
| GLU | OE2 | B | 240 | 8.3 | 16.7 | 8.1 | 36 |
| GLU | C | B | 240 | 13.1 | 15.6 | 9.3 | 27 |
| GLU | O | B | 240 | 13.2 | 14.4 | 9.7 | 31 |
| VAL | N | B | 241 | 13.1 | 16.6 | 10.1 | 24 |
| VAL | CA | B | 241 | 13.1 | 16.4 | 11.6 | 24 |
| VAL | CB | B | 241 | 14.4 | 16.9 | 12.2 | 27 |
| VAL | CG1 | B | 241 | 15.6 | 16.1 | 11.6 | 22 |
| VAL | CG2 | B | 241 | 14.7 | 18.4 | 11.9 | 21 |
| VAL | C | B | 241 | 11.9 | 17.2 | 12.2 | 24 |
| VAL | O | B | 241 | 11.2 | 17.9 | 11.5 | 20 |
| ILE | N | B | 242 | 11.7 | 17.0 | 13.5 | 23 |
| ILE | CA | B | 242 | 10.6 | 17.7 | 14.1 | 28 |
| ILE | CB | B | 242 | 9.5 | 16.7 | 14.6 | 29 |
| ILE | CG2 | B | 242 | 8.5 | 17.4 | 15.4 | 28 |
| ILE | CG1 | B | 242 | 8.9 | 16.0 | 13.4 | 33 |
| ILE | CD1 | B | 242 | 7.9 | 15.0 | 13.8 | 32 |
| ILE | C | B | 242 | 11.1 | 18.5 | 15.3 | 26 |
| ILE | O | B | 242 | 11.7 | 18.0 | 16.3 | 25 |
| ILE | N | B | 243 | 10.9 | 19.8 | 15.3 | 28 |
| ILE | CA | B | 243 | 11.2 | 20.7 | 16.3 | 24 |
| ILE | CB | B | 243 | 11.6 | 22.1 | 15.8 | 24 |
| ILE | CG2 | B | 243 | 11.9 | 23.1 | 16.9 | 21 |
| ILE | CG1 | B | 243 | 12.7 | 22.0 | 14.8 | 25 |
| ILE | CD1 | B | 243 | 13.1 | 23.4 | 14.1 | 23 |
| ILE | C | B | 243 | 10.1 | 20.8 | 17.3 | 25 |
| ILE | O | B | 243 | 8.9 | 21.1 | 16.9 | 26 |
| VAL | N | B | 244 | 10.3 | 20.6 | 18.6 | 24 |
| VAL | CA | B | 244 | 9.2 | 20.7 | 19.5 | 28 |
| VAL | CB | B | 244 | 9.2 | 19.3 | 20.3 | 30 |
| VAL | CG1 | B | 244 | 9.0 | 18.1 | 19.4 | 26 |
| VAL | CG2 | B | 244 | 10.4 | 19.2 | 21.2 | 28 |
| VAL | C | B | 244 | 9.2 | 21.8 | 20.5 | 29 |
| VAL | O | B | 244 | 8.2 | 22.0 | 21.3 | 31 |
| ARG | N | B | 245 | 10.2 | 22.7 | 20.5 | 29 |
| ARG | CA | B | 245 | 10.3 | 23.8 | 21.4 | 24 |
| ARG | CB | B | 245 | 10.6 | 23.4 | 22.8 | 26 |
| ARG | CG | B | 245 | 10.8 | 24.5 | 23.8 | 28 |
| ARG | CD | B | 245 | 11.1 | 23.9 | 25.2 | 26 |
| ARG | NE | B | 245 | 11.4 | 24.9 | 26.3 | 30 |
| ARG | CZ | B | 245 | 10.5 | 25.6 | 27.0 | 32 |
| ARG | NH1 | B | 245 | 9.2 | 25.4 | 26.8 | 30 |
| ARG | NH2 | B | 245 | 10.9 | 26.4 | 27.9 | 31 |
| ARG | C | B | 245 | 11.5 | 24.8 | 20.9 | 28 |
| ARG | O | B | 245 | 12.5 | 24.3 | 20.5 | 30 |
| VAL | N | B | 246 | 11.2 | 26.1 | 21.0 | 24 |
| VAL | CA | B | 246 | 12.2 | 27.1 | 20.7 | 21 |
| VAL | CB | B | 246 | 11.8 | 27.8 | 19.4 | 20 |
| VAL | CG1 | B | 246 | 12.9 | 28.8 | 19.0 | 20 |
| VAL | CG2 | B | 246 | 11.6 | 26.8 | 18.2 | 18 |
| VAL | C | B | 246 | 12.4 | 28.1 | 21.8 | 22 |
| VAL | O | B | 246 | 11.5 | 28.6 | 22.4 | 19 |
| GLU | N | B | 247 | 13.7 | 28.4 | 22.1 | 21 |
| GLU | CA | B | 247 | 14.1 | 29.3 | 23.1 | 22 |
| GLU | CB | B | 247 | 14.7 | 28.6 | 24.4 | 26 |
| GLU | CG | B | 247 | 13.7 | 27.6 | 25.1 | 28 |
| GLU | CD | B | 247 | 14.4 | 26.9 | 26.3 | 29 |
| GLU | OE1 | B | 247 | 15.6 | 27.2 | 26.5 | 30 |
| GLU | OE2 | B | 247 | 13.8 | 26.1 | 27.0 | 31 |
| GLU | C | B | 247 | 15.1 | 30.3 | 22.7 | 23 |
| GLU | O | B | 247 | 16.0 | 30.0 | 21.8 | 23 |
| ILE | N | B | 248 | 15.0 | 31.5 | 23.1 | 23 |
| ILE | CA | B | 248 | 16.0 | 32.6 | 22.8 | 22 |
| ILE | CB | B | 248 | 15.3 | 33.8 | 22.2 | 25 |
| ILE | CG2 | B | 248 | 16.3 | 35.0 | 21.9 | 24 |
| ILE | CG1 | B | 248 | 14.6 | 33.4 | 20.9 | 26 |
| ILE | CD1 | B | 248 | 15.4 | 32.8 | 19.8 | 23 |
| ILE | C | B | 248 | 16.5 | 32.9 | 24.2 | 21 |
| ILE | O | B | 248 | 15.6 | 33.3 | 25.1 | 20 |
| ASN | N | B | 249 | 17.8 | 32.7 | 24.6 | 21 |
| ASN | CA | B | 249 | 18.2 | 32.9 | 26.0 | 18 |
| ASN | CB | B | 249 | 18.5 | 34.4 | 26.4 | 18 |
| ASN | CG | B | 249 | 19.9 | 34.8 | 25.9 | 16 |
| ASN | OD1 | B | 249 | 20.7 | 34.0 | 25.4 | 17 |
| ASN | ND2 | B | 249 | 20.2 | 36.1 | 26.1 | 15 |
| ASN | C | B | 249 | 17.3 | 32.3 | 27.0 | 17 |
| ASN | O | B | 249 | 17.0 | 32.9 | 28.0 | 20 |
| GLY | N | B | 250 | 16.9 | 31.1 | 26.8 | 21 |
| GLY | CA | B | 250 | 16.1 | 30.4 | 27.7 | 18 |
| GLY | C | B | 250 | 14.6 | 30.8 | 27.7 | 22 |
| GLY | O | B | 250 | 13.8 | 30.1 | 28.4 | 23 |
| GLN | N | B | 251 | 14.2 | 31.8 | 27.0 | 24 |
| GLN | CA | B | 251 | 12.8 | 32.2 | 27.0 | 28 |
| GLN | CB | B | 251 | 12.7 | 33.8 | 27.0 | 23 |
| GLN | CG | B | 251 | 11.2 | 34.2 | 27.1 | 29 |
| GLN | CD | B | 251 | 11.1 | 35.7 | 27.0 | 33 |
| GLN | OE1 | B | 251 | 12.0 | 36.4 | 26.6 | 38 |
| GLN | NE2 | B | 251 | 9.9 | 36.2 | 27.4 | 32 |
| GLN | C | B | 251 | 12.0 | 31.5 | 26.0 | 23 |
| GLN | O | B | 251 | 12.3 | 31.7 | 24.8 | 24 |
| ASP | N | B | 252 | 11.1 | 30.7 | 26.4 | 25 |
| ASP | CA | B | 252 | 10.2 | 30.0 | 25.5 | 26 |
| ASP | CB | B | 252 | 9.1 | 29.3 | 26.4 | 27 |
| ASP | CG | B | 252 | 8.2 | 28.4 | 25.5 | 29 |
| ASP | OD1 | B | 252 | 8.4 | 28.2 | 24.3 | 31 |
| ASP | OD2 | B | 252 | 7.2 | 27.9 | 26.1 | 33 |
| ASP | C | B | 252 | 9.6 | 30.9 | 24.5 | 25 |
| ASP | O | B | 252 | 9.0 | 32.0 | 24.8 | 24 |
| LEU | N | B | 253 | 9.7 | 30.5 | 23.2 | 25 |
| LEU | CA | B | 253 | 9.2 | 31.4 | 22.2 | 30 |
| LEU | CB | B | 253 | 9.8 | 30.9 | 20.8 | 31 |
| LEU | CG | B | 253 | 9.5 | 31.9 | 19.7 | 35 |
| LEU | CD1 | B | 253 | 9.9 | 33.3 | 20.0 | 32 |
| LEU | CD2 | B | 253 | 10.1 | 31.4 | 18.4 | 31 |
| LEU | C | B | 253 | 7.6 | 31.2 | 22.2 | 32 |
| LEU | O | B | 253 | 6.9 | 32.0 | 21.6 | 31 |
| LYS | N | B | 254 | 7.2 | 30.3 | 23.0 | 33 |
| LYS | CA | B | 254 | 5.8 | 29.9 | 23.3 | 40 |
| LYS | CB | B | 254 | 5.3 | 30.8 | 24.5 | 46 |
| LYS | CG | B | 254 | 3.9 | 30.6 | 25.0 | 52 |
| LYS | CD | B | 254 | 3.6 | 31.5 | 26.1 | 56 |
| LYS | CE | B | 254 | 2.2 | 31.2 | 26.7 | 60 |
| LYS | NZ | B | 254 | 1.8 | 32.1 | 27.8 | 62 |
| LYS | C | B | 254 | 4.8 | 29.9 | 22.1 | 42 |
| LYS | O | B | 254 | 3.7 | 30.4 | 22.2 | 45 |
| MET | N | B | 255 | 5.1 | 29.2 | 21.1 | 41 |
| MET | CA | B | 255 | 4.3 | 29.0 | 19.9 | 39 |
| MET | CB | B | 255 | 5.0 | 29.4 | 18.6 | 37 |
| MET | CG | B | 255 | 5.4 | 30.9 | 18.5 | 35 |
| MET | SD | B | 255 | 6.2 | 31.1 | 16.9 | 36 |
| MET | CE | B | 255 | 4.8 | 31.3 | 15.8 | 38 |
| MET | C | B | 255 | 3.8 | 27.5 | 19.8 | 42 |
| MET | O | B | 255 | 4.5 | 26.7 | 20.3 | 39 |
| ASP | N | B | 256 | 2.7 | 27.3 | 19.2 | 45 |
| ASP | CA | B | 256 | 2.2 | 25.9 | 18.9 | 45 |
| ASP | CB | B | 256 | 0.9 | 25.9 | 18.0 | 52 |
| ASP | CG | B | 256 | 0.4 | 24.5 | 17.7 | 56 |
| ASP | OD1 | B | 256 | 1.1 | 23.6 | 18.1 | 60 |
| ASP | OD2 | B | 256 | −0.6 | 24.4 | 17.0 | 61 |
| ASP | C | B | 256 | 3.4 | 25.3 | 18.2 | 40 |
| ASP | O | B | 256 | 3.8 | 25.8 | 17.1 | 38 |
| CYS | N | B | 257 | 3.9 | 24.2 | 18.7 | 36 |
| CYS | CA | B | 257 | 5.1 | 23.6 | 18.1 | 38 |
| CYS | C | B | 257 | 4.9 | 23.3 | 16.7 | 38 |
| CYS | O | B | 257 | 5.9 | 23.0 | 15.9 | 39 |
| CYS | CB | B | 257 | 5.6 | 22.4 | 18.9 | 37 |
| CYS | SG | B | 257 | 4.3 | 21.1 | 19.0 | 45 |
| LYS | N | B | 258 | 3.7 | 23.2 | 16.2 | 39 |
| LYS | CA | B | 258 | 3.4 | 22.9 | 14.8 | 37 |
| LYS | CB | B | 258 | 2.0 | 22.4 | 14.7 | 39 |
| LYS | CG | B | 258 | 1.6 | 22.0 | 13.3 | 45 |
| LYS | CD | B | 258 | 0.1 | 21.6 | 13.3 | 48 |
| LYS | CE | B | 258 | −0.1 | 20.4 | 14.2 | 50 |
| LYS | NZ | B | 258 | −1.5 | 19.9 | 14.3 | 52 |
| LYS | C | B | 258 | 3.8 | 24.0 | 13.9 | 36 |
| LYS | O | B | 258 | 4.1 | 23.8 | 12.7 | 34 |
| GLU | N | B | 259 | 4.0 | 25.2 | 14.4 | 34 |
| GLU | CA | B | 259 | 4.4 | 26.4 | 13.6 | 32 |
| GLU | CB | B | 259 | 4.1 | 27.7 | 14.3 | 33 |
| GLU | CG | B | 259 | 2.7 | 28.0 | 14.6 | 36 |
| GLU | CD | B | 259 | 1.9 | 28.2 | 13.3 | 42 |
| GLU | OE1 | B | 259 | 2.5 | 28.2 | 12.3 | 43 |
| GLU | OE2 | B | 259 | 0.6 | 28.3 | 13.4 | 43 |
| GLU | C | B | 259 | 5.9 | 26.3 | 13.3 | 31 |
| GLU | O | B | 259 | 6.4 | 26.8 | 12.3 | 30 |
| TYR | N | B | 260 | 6.6 | 25.6 | 14.1 | 28 |
| TYR | CA | B | 260 | 8.1 | 25.4 | 14.0 | 27 |
| TYR | CB | B | 260 | 8.7 | 24.8 | 15.3 | 20 |
| TYR | CG | B | 260 | 8.4 | 25.7 | 16.5 | 20 |
| TYR | CD1 | B | 260 | 8.2 | 25.0 | 17.7 | 21 |
| TYR | CE1 | B | 260 | 8.0 | 25.8 | 18.9 | 19 |
| TYR | CD2 | B | 260 | 8.5 | 27.0 | 16.5 | 16 |
| TYR | CE2 | B | 260 | 8.3 | 27.8 | 17.6 | 17 |
| TYR | CZ | B | 260 | 8.1 | 27.2 | 18.8 | 21 |
| TYR | OH | B | 260 | 7.9 | 27.9 | 20.0 | 21 |
| TYR | C | B | 260 | 8.4 | 24.4 | 12.8 | 30 |
| TYR | O | B | 260 | 9.5 | 24.3 | 12.4 | 30 |
| ASN | N | B | 261 | 7.3 | 23.7 | 12.4 | 32 |
| ASN | CA | B | 261 | 7.5 | 22.7 | 11.4 | 33 |
| ASN | CB | B | 261 | 7.3 | 21.3 | 12.0 | 32 |
| ASN | CG | B | 261 | 8.3 | 21.1 | 13.1 | 33 |
| ASN | OD1 | B | 261 | 9.5 | 21.3 | 13.0 | 30 |
| ASN | ND2 | B | 261 | 7.8 | 20.6 | 14.3 | 34 |
| ASN | C | B | 261 | 6.5 | 22.9 | 10.2 | 31 |
| ASN | O | B | 261 | 6.3 | 21.9 | 9.4 | 29 |
| TYR | N | B | 262 | 6.0 | 24.1 | 10.0 | 32 |
| TYR | CA | B | 262 | 5.1 | 24.4 | 9.0 | 34 |
| TYR | CB | B | 262 | 4.1 | 25.5 | 9.4 | 37 |
| TYR | CG | B | 262 | 3.2 | 26.0 | 8.4 | 42 |
| TYR | CD1 | B | 262 | 2.3 | 25.1 | 7.7 | 41 |
| TYR | CE1 | B | 262 | 1.4 | 25.6 | 6.7 | 46 |
| TYR | CD2 | B | 262 | 3.1 | 27.3 | 8.0 | 43 |
| TYR | CE2 | B | 262 | 2.3 | 27.8 | 7.1 | 46 |
| TYR | CZ | B | 262 | 1.4 | 26.9 | 6.4 | 47 |
| TYR | OH | B | 262 | 0.6 | 27.4 | 5.4 | 48 |
| TYR | C | B | 262 | 5.9 | 25.0 | 7.8 | 34 |
| TYR | O | B | 262 | 6.5 | 26.0 | 7.8 | 37 |
| ASP | N | B | 263 | 5.9 | 24.3 | 6.6 | 32 |
| ASP | CA | B | 263 | 5.2 | 23.0 | 6.5 | 35 |
| ASP | CB | B | 263 | 4.5 | 22.9 | 5.1 | 39 |
| ASP | CG | B | 263 | 5.5 | 23.0 | 3.9 | 44 |
| ASP | OD1 | B | 263 | 6.7 | 23.2 | 4.1 | 46 |
| ASP | OD2 | B | 263 | 5.0 | 22.9 | 2.8 | 48 |
| ASP | C | B | 263 | 6.2 | 21.8 | 6.7 | 34 |
| ASP | O | B | 263 | 5.8 | 20.6 | 6.6 | 31 |
| LYS | N | B | 264 | 7.4 | 22.1 | 7.0 | 30 |
| LYS | CA | B | 264 | 8.4 | 21.1 | 7.3 | 30 |
| LYS | CB | B | 264 | 8.7 | 20.3 | 6.0 | 27 |
| LYS | CG | B | 264 | 9.3 | 21.1 | 4.9 | 26 |
| LYS | CD | B | 264 | 9.6 | 20.2 | 3.7 | 24 |
| LYS | CE | B | 264 | 10.2 | 21.1 | 2.6 | 31 |
| LYS | NZ | B | 264 | 9.3 | 22.1 | 2.1 | 31 |
| LYS | C | B | 264 | 9.7 | 21.7 | 7.8 | 27 |
| LYS | O | B | 264 | 9.9 | 22.9 | 7.7 | 29 |
| SER | N | B | 265 | 10.5 | 20.8 | 8.5 | 23 |
| SER | CA | B | 265 | 11.8 | 21.2 | 9.1 | 26 |
| SER | CB | B | 265 | 11.8 | 21.2 | 10.6 | 25 |
| SER | OG | B | 265 | 10.9 | 22.2 | 11.1 | 28 |
| SER | C | B | 265 | 12.8 | 20.3 | 8.5 | 23 |
| SER | O | B | 265 | 12.7 | 19.1 | 8.6 | 24 |
| ILE | N | B | 266 | 13.8 | 20.8 | 7.8 | 24 |
| ILE | CA | B | 266 | 14.9 | 20.0 | 7.2 | 24 |
| ILE | CB | B | 266 | 14.8 | 20.0 | 5.7 | 23 |
| ILE | CG2 | B | 266 | 13.4 | 19.5 | 5.3 | 21 |
| ILE | CG1 | B | 266 | 15.1 | 21.3 | 5.1 | 26 |
| ILE | CD1 | B | 266 | 15.1 | 21.4 | 3.5 | 22 |
| ILE | C | B | 266 | 16.3 | 20.5 | 7.6 | 25 |
| ILE | O | B | 266 | 16.5 | 21.6 | 8.0 | 20 |
| VAL | N | B | 267 | 17.2 | 19.6 | 7.4 | 22 |
| VAL | CA | B | 267 | 18.6 | 19.9 | 7.7 | 26 |
| VAL | CB | B | 267 | 19.3 | 18.8 | 8.5 | 24 |
| VAL | CG1 | B | 267 | 20.8 | 19.2 | 8.8 | 28 |
| VAL | CG2 | B | 267 | 18.6 | 18.5 | 9.8 | 18 |
| VAL | C | B | 267 | 19.2 | 20.0 | 6.3 | 29 |
| VAL | O | B | 267 | 19.3 | 19.0 | 5.6 | 26 |
| ASP | N | B | 268 | 19.7 | 21.2 | 6.0 | 27 |
| ASP | CA | B | 268 | 20.3 | 21.4 | 4.6 | 25 |
| ASP | CB | B | 268 | 19.3 | 22.2 | 3.8 | 23 |
| ASP | CG | B | 268 | 19.7 | 22.4 | 2.4 | 26 |
| ASP | OD1 | B | 268 | 20.7 | 21.8 | 2.0 | 24 |
| ASP | OD2 | B | 268 | 19.2 | 23.3 | 1.7 | 29 |
| ASP | C | B | 268 | 21.7 | 22.1 | 4.6 | 21 |
| ASP | O | B | 268 | 21.8 | 23.3 | 4.8 | 24 |
| SER | N | B | 269 | 22.7 | 21.3 | 4.2 | 20 |
| SER | CA | B | 269 | 24.0 | 21.8 | 4.1 | 16 |
| SER | CB | B | 269 | 25.0 | 20.6 | 3.9 | 17 |
| SER | OG | B | 269 | 24.7 | 19.9 | 2.7 | 18 |
| SER | C | B | 269 | 24.2 | 22.8 | 3.0 | 18 |
| SER | O | B | 269 | 25.2 | 23.5 | 2.9 | 17 |
| GLY | N | B | 270 | 23.2 | 22.8 | 2.1 | 18 |
| GLY | CA | B | 270 | 23.3 | 23.7 | 0.9 | 18 |
| GLY | C | B | 270 | 22.6 | 25.0 | 1.2 | 19 |
| GLY | O | B | 270 | 22.4 | 25.8 | 0.2 | 21 |
| THR | N | B | 271 | 22.2 | 25.3 | 2.4 | 17 |
| THR | CA | B | 271 | 21.6 | 26.5 | 2.8 | 20 |
| THR | CB | B | 271 | 20.2 | 26.4 | 3.4 | 20 |
| THR | OG1 | B | 271 | 19.3 | 25.8 | 2.4 | 22 |
| THR | CG2 | B | 271 | 19.6 | 27.7 | 3.9 | 19 |
| THR | C | B | 271 | 22.5 | 27.3 | 3.8 | 19 |
| THR | O | B | 271 | 22.9 | 26.7 | 4.8 | 20 |
| THR | N | B | 272 | 22.8 | 28.5 | 3.5 | 24 |
| THR | CA | B | 272 | 23.6 | 29.4 | 4.3 | 22 |
| THR | CB | B | 272 | 24.0 | 30.7 | 3.7 | 21 |
| THR | OG1 | B | 272 | 24.6 | 30.4 | 2.4 | 25 |
| THR | CG2 | B | 272 | 24.9 | 31.5 | 4.6 | 22 |
| THR | C | B | 272 | 23.0 | 29.7 | 5.7 | 21 |
| THR | O | B | 272 | 23.6 | 29.5 | 6.7 | 21 |
| ASN | N | B | 273 | 21.7 | 30.2 | 5.7 | 24 |
| ASN | CA | B | 273 | 21.0 | 30.6 | 6.9 | 21 |
| ASN | CB | B | 273 | 20.1 | 31.7 | 6.5 | 23 |
| ASN | CG | B | 273 | 20.8 | 32.9 | 6.0 | 23 |
| ASN | OD1 | B | 273 | 22.1 | 32.9 | 5.9 | 22 |
| ASN | ND2 | B | 273 | 20.1 | 34.0 | 5.7 | 24 |
| ASN | C | B | 273 | 20.2 | 29.5 | 7.6 | 22 |
| ASN | O | B | 273 | 20.1 | 28.4 | 7.2 | 21 |
| LEU | N | B | 274 | 19.6 | 30.1 | 8.7 | 20 |
| LEU | CA | B | 274 | 18.7 | 29.4 | 9.5 | 24 |
| LEU | CB | B | 274 | 18.8 | 29.7 | 11.0 | 21 |
| LEU | CG | B | 274 | 17.7 | 29.1 | 11.8 | 20 |
| LEU | CD1 | B | 274 | 17.6 | 27.5 | 11.7 | 17 |
| LEU | CD2 | B | 274 | 17.9 | 29.4 | 13.3 | 19 |
| LEU | C | B | 274 | 17.4 | 30.0 | 9.0 | 26 |
| LEU | O | B | 274 | 17.2 | 31.2 | 9.1 | 21 |
| ARG | N | B | 275 | 16.6 | 29.2 | 8.2 | 23 |
| ARG | CA | B | 275 | 15.4 | 29.8 | 7.7 | 23 |
| ARG | CB | B | 275 | 15.2 | 29.6 | 6.2 | 25 |
| ARG | CG | B | 275 | 16.4 | 30.2 | 5.5 | 29 |
| ARG | CD | B | 275 | 16.3 | 30.1 | 4.0 | 32 |
| ARG | NE | B | 275 | 15.1 | 30.8 | 3.6 | 35 |
| ARG | CZ | B | 275 | 14.9 | 31.4 | 2.4 | 35 |
| ARG | NH1 | B | 275 | 13.8 | 32.0 | 2.2 | 34 |
| ARG | NH2 | B | 275 | 15.9 | 31.4 | 1.6 | 37 |
| ARG | C | B | 275 | 14.2 | 29.3 | 8.6 | 25 |
| ARG | O | B | 275 | 14.1 | 28.2 | 8.9 | 22 |
| LEU | N | B | 276 | 13.4 | 30.3 | 8.9 | 24 |
| LEU | CA | B | 276 | 12.2 | 30.1 | 9.7 | 25 |
| LEU | CB | B | 276 | 12.3 | 30.9 | 11.1 | 23 |
| LEU | CG | B | 276 | 13.5 | 30.5 | 11.9 | 24 |
| LEU | CD1 | B | 276 | 13.7 | 31.4 | 13.1 | 22 |
| LEU | CD2 | B | 276 | 13.5 | 29.0 | 12.3 | 23 |
| LEU | C | B | 276 | 10.9 | 30.5 | 9.1 | 25 |
| LEU | O | B | 276 | 10.8 | 31.5 | 8.4 | 27 |
| PRO | N | B | 277 | 9.8 | 29.7 | 9.3 | 28 |
| PRO | CD | B | 277 | 9.9 | 28.3 | 9.8 | 30 |
| PRO | CA | B | 277 | 8.5 | 29.9 | 8.7 | 30 |
| PRO | CB | B | 277 | 7.7 | 28.9 | 9.4 | 32 |
| PRO | CG | B | 277 | 8.6 | 27.7 | 9.2 | 33 |
| PRO | C | B | 277 | 8.1 | 31.3 | 9.1 | 31 |
| PRO | O | B | 277 | 8.3 | 31.7 | 10.3 | 31 |
| LYS | N | B | 278 | 7.6 | 32.1 | 8.2 | 34 |
| LYS | CA | B | 278 | 7.1 | 33.5 | 8.5 | 36 |
| LYS | CB | B | 278 | 6.0 | 33.9 | 7.5 | 41 |
| LYS | CG | B | 278 | 5.4 | 35.3 | 7.8 | 47 |
| LYS | CD | B | 278 | 6.5 | 36.4 | 7.6 | 53 |
| LYS | CE | B | 278 | 5.9 | 37.8 | 7.8 | 57 |
| LYS | NZ | B | 278 | 7.0 | 38.8 | 7.6 | 55 |
| LYS | C | B | 278 | 6.7 | 33.9 | 9.9 | 33 |
| LYS | O | B | 278 | 7.4 | 34.7 | 10.5 | 32 |
| LYS | N | B | 279 | 5.7 | 33.2 | 10.5 | 32 |
| LYS | CA | B | 279 | 5.3 | 33.5 | 11.8 | 37 |
| LYS | CB | B | 279 | 4.0 | 32.7 | 12.2 | 40 |
| LYS | CG | B | 279 | 2.8 | 33.1 | 11.3 | 51 |
| LYS | CD | B | 279 | 1.6 | 32.2 | 11.7 | 54 |
| LYS | CE | B | 279 | 1.2 | 32.5 | 13.2 | 57 |
| LYS | NZ | B | 279 | 0.0 | 31.6 | 13.5 | 62 |
| LYS | C | B | 279 | 6.4 | 33.2 | 12.9 | 33 |
| LYS | O | B | 279 | 6.4 | 33.9 | 13.9 | 32 |
| VAL | N | B | 280 | 7.2 | 32.3 | 12.7 | 29 |
| VAL | CA | B | 280 | 8.3 | 31.9 | 13.6 | 26 |
| VAL | CB | B | 280 | 8.8 | 30.5 | 13.5 | 24 |
| VAL | CG1 | B | 280 | 9.9 | 30.2 | 14.5 | 25 |
| VAL | CG2 | B | 280 | 7.6 | 29.5 | 13.6 | 23 |
| VAL | C | B | 280 | 9.4 | 32.9 | 13.5 | 26 |
| VAL | O | B | 280 | 10.1 | 33.3 | 14.5 | 27 |
| PHE | N | B | 281 | 9.7 | 33.4 | 12.3 | 24 |
| PHE | CA | B | 281 | 10.7 | 34.3 | 12.1 | 29 |
| PHE | CB | B | 281 | 10.9 | 34.6 | 10.6 | 28 |
| PHE | CG | B | 281 | 12.0 | 35.6 | 10.3 | 29 |
| PHE | CD1 | B | 281 | 13.3 | 35.3 | 10.4 | 31 |
| PHE | CD2 | B | 281 | 11.6 | 36.9 | 9.9 | 28 |
| PHE | CE1 | B | 281 | 14.3 | 36.2 | 10.2 | 31 |
| PHE | CE2 | B | 281 | 12.6 | 37.8 | 9.6 | 32 |
| PHE | CZ | B | 281 | 14.0 | 37.5 | 9.8 | 31 |
| PHE | C | B | 281 | 10.4 | 35.6 | 12.8 | 31 |
| PHE | O | B | 281 | 11.2 | 36.2 | 13.5 | 29 |
| GLU | N | B | 282 | 9.1 | 36.1 | 12.6 | 34 |
| GLU | CA | B | 282 | 8.6 | 37.3 | 13.2 | 36 |
| GLU | CB | B | 282 | 7.2 | 37.6 | 12.8 | 41 |
| GLU | CG | B | 282 | 6.9 | 37.8 | 11.3 | 54 |
| GLU | CD | B | 282 | 7.7 | 38.9 | 10.7 | 62 |
| GLU | OE1 | B | 282 | 7.1 | 39.9 | 10.2 | 69 |
| GLU | OE2 | B | 282 | 9.0 | 38.9 | 10.7 | 65 |
| GLU | C | B | 282 | 8.7 | 37.2 | 14.7 | 33 |
| GLU | O | B | 282 | 9.1 | 38.2 | 15.3 | 34 |
| ALA | N | B | 283 | 8.5 | 36.1 | 15.3 | 29 |
| ALA | CA | B | 283 | 8.5 | 35.9 | 16.7 | 27 |
| ALA | CB | B | 283 | 7.7 | 34.7 | 17.1 | 26 |
| ALA | C | B | 283 | 10.0 | 35.8 | 17.2 | 31 |
| ALA | O | B | 283 | 10.3 | 36.2 | 18.3 | 34 |
| ALA | N | B | 284 | 10.8 | 35.2 | 16.4 | 28 |
| ALA | CA | B | 284 | 12.2 | 35.0 | 16.8 | 27 |
| ALA | CB | B | 284 | 12.9 | 33.9 | 15.9 | 22 |
| ALA | C | B | 284 | 13.0 | 36.3 | 16.7 | 26 |
| ALA | O | B | 284 | 13.7 | 36.6 | 17.6 | 26 |
| VAL | N | B | 285 | 12.7 | 37.1 | 15.7 | 24 |
| VAL | CA | B | 285 | 13.4 | 38.4 | 15.5 | 27 |
| VAL | CB | B | 285 | 13.2 | 39.0 | 14.1 | 27 |
| VAL | CG1 | B | 285 | 13.9 | 40.3 | 14.0 | 32 |
| VAL | CG2 | B | 285 | 13.8 | 38.0 | 13.0 | 32 |
| VAL | C | B | 285 | 13.0 | 39.4 | 16.6 | 27 |
| VAL | O | B | 285 | 13.7 | 40.2 | 17.0 | 28 |
| LYS | N | B | 286 | 11.7 | 39.3 | 16.9 | 26 |
| LYS | CA | B | 286 | 11.2 | 40.2 | 18.0 | 24 |
| LYS | CB | B | 286 | 9.7 | 40.1 | 18.1 | 25 |
| LYS | CG | B | 286 | 9.1 | 41.0 | 19.2 | 28 |
| LYS | CD | B | 286 | 7.6 | 40.9 | 19.4 | 30 |
| LYS | CE | B | 286 | 7.1 | 41.8 | 20.4 | 37 |
| LYS | NZ | B | 286 | 5.6 | 41.7 | 20.6 | 41 |
| LYS | C | B | 286 | 11.9 | 39.9 | 19.3 | 25 |
| LYS | O | B | 286 | 12.3 | 40.8 | 20.0 | 23 |
| SER | N | B | 287 | 12.0 | 38.6 | 19.6 | 22 |
| SER | CA | B | 287 | 12.7 | 38.2 | 20.8 | 21 |
| SER | CB | B | 287 | 12.4 | 36.8 | 21.1 | 22 |
| SER | OG | B | 287 | 13.0 | 36.3 | 22.3 | 24 |
| SER | C | B | 287 | 14.2 | 38.4 | 20.8 | 23 |
| SER | O | B | 287 | 14.8 | 38.7 | 21.8 | 27 |
| ILE | N | B | 288 | 14.8 | 38.3 | 19.6 | 24 |
| ILE | CA | B | 288 | 16.2 | 38.5 | 19.4 | 26 |
| ILE | CB | B | 288 | 16.8 | 37.9 | 18.2 | 27 |
| ILE | CG2 | B | 288 | 18.2 | 38.3 | 18.0 | 24 |
| ILE | CG1 | B | 288 | 16.5 | 36.4 | 18.2 | 24 |
| ILE | CD1 | B | 288 | 17.0 | 35.7 | 16.9 | 23 |
| ILE | C | B | 288 | 16.6 | 40.0 | 19.5 | 27 |
| ILE | O | B | 288 | 17.7 | 40.4 | 20.0 | 30 |
| LYS | N | B | 289 | 15.6 | 40.9 | 19.2 | 26 |
| LYS | CA | B | 289 | 15.9 | 42.3 | 19.3 | 25 |
| LYS | CB | B | 289 | 14.9 | 43.2 | 18.4 | 25 |
| LYS | CG | B | 289 | 14.9 | 42.9 | 16.9 | 32 |
| LYS | CD | B | 289 | 13.9 | 43.9 | 16.3 | 33 |
| LYS | CE | B | 289 | 13.9 | 43.7 | 14.8 | 37 |
| LYS | NZ | B | 289 | 12.8 | 44.7 | 14.2 | 38 |
| LYS | C | B | 289 | 15.8 | 42.8 | 20.7 | 23 |
| LYS | O | B | 289 | 16.6 | 43.6 | 21.2 | 25 |
| ALA | N | B | 290 | 14.8 | 42.2 | 21.4 | 18 |
| ALA | CA | B | 290 | 14.6 | 42.6 | 22.8 | 22 |
| ALA | CB | B | 290 | 13.3 | 41.9 | 23.3 | 13 |
| ALA | C | B | 290 | 15.8 | 42.2 | 23.7 | 20 |
| ALA | O | B | 290 | 16.1 | 42.9 | 24.6 | 21 |
| ALA | N | B | 291 | 16.3 | 41.0 | 23.4 | 20 |
| ALA | CA | B | 291 | 17.5 | 40.5 | 24.2 | 24 |
| ALA | CB | B | 291 | 17.7 | 39.0 | 23.9 | 21 |
| ALA | C | B | 291 | 18.7 | 41.3 | 23.9 | 22 |
| ALA | O | B | 291 | 19.6 | 41.4 | 24.8 | 24 |
| SER | N | B | 292 | 18.9 | 41.8 | 22.7 | 23 |
| SER | CA | B | 292 | 20.0 | 42.6 | 22.3 | 26 |
| SER | CB | B | 292 | 20.5 | 42.2 | 20.9 | 27 |
| SER | OG | B | 292 | 19.5 | 42.4 | 19.9 | 28 |
| SER | C | B | 292 | 19.8 | 44.1 | 22.4 | 26 |
| SER | O | B | 292 | 20.7 | 44.9 | 21.9 | 25 |
| SER | N | B | 293 | 18.7 | 44.5 | 22.9 | 25 |
| SER | CA | B | 293 | 18.3 | 45.9 | 23.0 | 27 |
| SER | CB | B | 293 | 16.9 | 46.0 | 23.7 | 27 |
| SER | OG | B | 293 | 16.9 | 45.5 | 25.0 | 35 |
| SER | C | B | 293 | 19.2 | 46.9 | 23.6 | 30 |
| SER | O | B | 293 | 19.0 | 48.1 | 23.6 | 31 |
| THR | N | B | 294 | 20.4 | 46.5 | 24.2 | 29 |
| THR | CA | B | 294 | 21.3 | 47.5 | 24.7 | 30 |
| THR | CB | B | 294 | 22.4 | 46.9 | 25.7 | 31 |
| THR | OG1 | B | 294 | 23.2 | 46.0 | 24.9 | 32 |
| THR | CG2 | B | 294 | 21.8 | 46.3 | 26.9 | 34 |
| THR | C | B | 294 | 21.9 | 48.2 | 23.6 | 28 |
| THR | O | B | 294 | 22.6 | 49.2 | 23.7 | 26 |
| GLU | N | B | 295 | 21.7 | 47.7 | 22.4 | 29 |
| GLU | CA | B | 295 | 22.2 | 48.4 | 21.2 | 34 |
| GLU | CB | B | 295 | 23.6 | 47.9 | 20.8 | 32 |
| GLU | CG | B | 295 | 24.0 | 48.6 | 19.5 | 36 |
| GLU | CD | B | 295 | 25.4 | 48.1 | 19.1 | 42 |
| GLU | OE1 | B | 295 | 26.1 | 47.4 | 19.9 | 46 |
| GLU | OE2 | B | 295 | 25.8 | 48.4 | 18.0 | 43 |
| GLU | C | B | 295 | 21.2 | 48.2 | 20.1 | 34 |
| GLU | O | B | 295 | 20.7 | 47.1 | 19.8 | 38 |
| LYS | N | B | 296 | 20.8 | 49.3 | 19.5 | 34 |
| LYS | CA | B | 296 | 19.8 | 49.3 | 18.4 | 33 |
| LYS | CB | B | 296 | 18.8 | 50.5 | 18.6 | 38 |
| LYS | CG | B | 296 | 17.7 | 50.6 | 17.6 | 47 |
| LYS | CD | B | 296 | 16.8 | 51.8 | 17.9 | 52 |
| LYS | CE | B | 296 | 15.6 | 51.9 | 16.9 | 56 |
| LYS | NZ | B | 296 | 14.7 | 53.1 | 17.3 | 60 |
| LYS | C | B | 296 | 20.4 | 49.3 | 17.0 | 27 |
| LYS | O | B | 296 | 21.3 | 50.1 | 16.7 | 28 |
| PHE | N | B | 297 | 20.0 | 48.4 | 16.1 | 21 |
| PHE | CA | B | 297 | 20.5 | 48.3 | 14.8 | 24 |
| PHE | CB | B | 297 | 21.0 | 46.9 | 14.5 | 23 |
| PHE | CG | B | 297 | 22.0 | 46.4 | 15.5 | 24 |
| PHE | CD1 | B | 297 | 21.6 | 45.6 | 16.6 | 22 |
| PHE | CD2 | B | 297 | 23.3 | 46.7 | 15.4 | 26 |
| PHE | CE1 | B | 297 | 22.5 | 45.1 | 17.5 | 27 |
| PHE | CE2 | B | 297 | 24.2 | 46.3 | 16.3 | 24 |
| PHE | CZ | B | 297 | 23.8 | 45.5 | 17.4 | 25 |
| PHE | C | B | 297 | 19.5 | 48.7 | 13.7 | 26 |
| PHE | O | B | 297 | 18.3 | 48.7 | 13.9 | 27 |
| PRO | N | B | 298 | 20.0 | 49.2 | 12.5 | 25 |
| PRO | CD | B | 298 | 21.4 | 49.5 | 12.3 | 25 |
| PRO | CA | B | 298 | 19.3 | 49.6 | 11.4 | 26 |
| PRO | CB | B | 298 | 20.3 | 49.8 | 10.3 | 27 |
| PRO | CG | B | 298 | 21.4 | 50.5 | 11.1 | 26 |
| PRO | C | B | 298 | 18.3 | 48.5 | 11.0 | 26 |
| PRO | O | B | 298 | 18.6 | 47.3 | 11.1 | 24 |
| ASP | N | B | 299 | 17.1 | 48.8 | 10.5 | 27 |
| ASP | CA | B | 299 | 16.2 | 47.8 | 10.1 | 32 |
| ASP | CB | B | 299 | 14.8 | 48.3 | 9.8 | 38 |
| ASP | CG | B | 299 | 14.1 | 49.0 | 10.9 | 44 |
| ASP | OD1 | B | 299 | 14.7 | 49.0 | 12.0 | 46 |
| ASP | OD2 | B | 299 | 12.9 | 49.4 | 10.8 | 45 |
| ASP | C | B | 299 | 16.7 | 46.9 | 9.0 | 34 |
| ASP | O | B | 299 | 16.4 | 45.8 | 8.8 | 35 |
| GLY | N | B | 300 | 17.6 | 47.5 | 8.2 | 34 |
| GLY | CA | B | 300 | 18.2 | 46.8 | 7.1 | 31 |
| GLY | C | B | 300 | 18.9 | 45.5 | 7.6 | 34 |
| GLY | O | B | 300 | 18.9 | 44.5 | 7.0 | 32 |
| PHE | N | B | 301 | 19.6 | 45.7 | 8.8 | 30 |
| PHE | CA | B | 301 | 20.3 | 44.6 | 9.4 | 30 |
| PHE | CB | B | 301 | 20.9 | 45.0 | 10.8 | 30 |
| PHE | CG | B | 301 | 21.5 | 43.9 | 11.5 | 29 |
| PHE | CD1 | B | 301 | 22.7 | 43.3 | 11.1 | 30 |
| PHE | CD2 | B | 301 | 21.0 | 43.5 | 12.7 | 27 |
| PHE | CE1 | B | 301 | 23.3 | 42.3 | 11.8 | 29 |
| PHE | CE2 | B | 301 | 21.6 | 42.5 | 13.5 | 29 |
| PHE | CZ | B | 301 | 22.8 | 41.9 | 13.0 | 27 |
| PHE | C | B | 301 | 19.4 | 43.4 | 9.7 | 30 |
| PHE | O | B | 301 | 19.7 | 42.3 | 9.2 | 26 |
| TRP | N | B | 302 | 18.3 | 43.6 | 10.4 | 30 |
| TRP | CA | B | 302 | 17.4 | 42.5 | 10.7 | 28 |
| TRP | CB | B | 302 | 16.3 | 42.9 | 11.7 | 29 |
| TRP | CG | B | 302 | 16.9 | 43.4 | 12.9 | 26 |
| TRP | CD2 | B | 302 | 17.5 | 42.7 | 14.0 | 23 |
| TRP | CE2 | B | 302 | 17.9 | 43.6 | 15.0 | 23 |
| TRP | CE3 | B | 302 | 17.8 | 41.3 | 14.1 | 22 |
| TRP | CD1 | B | 302 | 16.9 | 44.7 | 13.4 | 26 |
| TRP | NE1 | B | 302 | 17.6 | 44.8 | 14.6 | 23 |
| TRP | CZ2 | B | 302 | 18.6 | 43.1 | 16.1 | 22 |
| TRP | CZ3 | B | 302 | 18.5 | 40.9 | 15.3 | 22 |
| TRP | CH2 | B | 302 | 18.9 | 41.8 | 16.3 | 23 |
| TRP | C | B | 302 | 16.8 | 41.8 | 9.5 | 30 |
| TRP | O | B | 302 | 16.3 | 40.7 | 9.5 | 24 |
| LEU | N | B | 303 | 16.9 | 42.5 | 8.3 | 28 |
| LEU | CA | B | 303 | 16.3 | 42.0 | 7.1 | 30 |
| LEU | CB | B | 303 | 15.7 | 43.1 | 6.2 | 33 |
| LEU | CG | B | 303 | 14.6 | 43.8 | 6.9 | 33 |
| LEU | CD1 | B | 303 | 14.1 | 45.0 | 6.0 | 34 |
| LEU | CD2 | B | 303 | 13.4 | 42.9 | 7.2 | 30 |
| LEU | C | B | 303 | 17.4 | 41.3 | 6.3 | 29 |
| LEU | O | B | 303 | 17.2 | 40.7 | 5.2 | 28 |
| GLY | N | B | 304 | 18.6 | 41.3 | 6.8 | 28 |
| GLY | CA | B | 304 | 19.7 | 40.7 | 6.1 | 31 |
| GLY | C | B | 304 | 20.1 | 41.4 | 4.8 | 32 |
| GLY | O | B | 304 | 20.6 | 40.9 | 3.9 | 34 |
| GLU | N | B | 305 | 19.9 | 42.7 | 4.8 | 33 |
| GLU | CA | B | 305 | 20.2 | 43.6 | 3.7 | 33 |
| GLU | CB | B | 305 | 19.0 | 44.4 | 3.3 | 35 |
| GLU | CG | B | 305 | 17.8 | 43.7 | 2.9 | 45 |
| GLU | CD | B | 305 | 16.7 | 44.7 | 2.5 | 53 |
| GLU | OE1 | B | 305 | 16.1 | 44.6 | 1.4 | 62 |
| GLU | OE2 | B | 305 | 16.4 | 45.5 | 3.3 | 55 |
| GLU | C | B | 305 | 21.4 | 44.5 | 4.0 | 33 |
| GLU | O | B | 305 | 22.0 | 45.1 | 3.1 | 34 |
| GLN | N | B | 306 | 21.8 | 44.5 | 5.3 | 29 |
| GLN | CA | B | 306 | 22.9 | 45.4 | 5.7 | 28 |
| GLN | CB | B | 306 | 22.3 | 46.7 | 6.3 | 27 |
| GLN | CG | B | 306 | 21.6 | 47.5 | 5.3 | 32 |
| GLN | CD | B | 306 | 21.1 | 48.8 | 5.9 | 34 |
| GLN | OE1 | B | 306 | 20.0 | 49.2 | 5.8 | 32 |
| GLN | NE2 | B | 306 | 21.9 | 49.4 | 6.8 | 36 |
| GLN | C | B | 306 | 23.8 | 44.7 | 6.8 | 28 |
| GLN | O | B | 306 | 23.4 | 43.9 | 7.6 | 30 |
| LEU | N | B | 307 | 25.1 | 45.1 | 6.6 | 24 |
| LEU | CA | B | 307 | 26.1 | 44.7 | 7.6 | 25 |
| LEU | CB | B | 307 | 27.5 | 44.7 | 6.9 | 21 |
| LEU | CG | B | 307 | 27.6 | 43.8 | 5.6 | 28 |
| LEU | CD1 | B | 307 | 29.0 | 44.0 | 5.1 | 22 |
| LEU | CD2 | B | 307 | 27.2 | 42.4 | 5.9 | 25 |
| LEU | C | B | 307 | 26.2 | 45.6 | 8.8 | 26 |
| LEU | O | B | 307 | 26.0 | 46.8 | 8.7 | 30 |
| VAL | N | B | 308 | 26.5 | 45.0 | 9.9 | 24 |
| VAL | CA | B | 308 | 26.7 | 45.8 | 11.2 | 25 |
| VAL | CB | B | 308 | 25.8 | 45.3 | 12.3 | 27 |
| VAL | CG1 | B | 308 | 26.2 | 46.0 | 13.6 | 30 |
| VAL | CG2 | B | 308 | 24.3 | 45.7 | 12.1 | 30 |
| VAL | C | B | 308 | 28.1 | 45.6 | 11.4 | 29 |
| VAL | O | B | 308 | 28.7 | 44.5 | 11.3 | 30 |
| CYS | N | B | 309 | 28.8 | 46.7 | 11.8 | 28 |
| CYS | CA | B | 309 | 30.2 | 46.7 | 12.1 | 26 |
| CYS | C | B | 309 | 30.6 | 47.2 | 13.5 | 28 |
| CYS | O | B | 309 | 29.9 | 48.0 | 14.0 | 29 |
| CYS | CB | B | 309 | 31.0 | 47.5 | 11.0 | 29 |
| CYS | SG | B | 309 | 30.7 | 47.1 | 9.3 | 33 |
| TRP | N | B | 310 | 31.7 | 46.6 | 14.0 | 25 |
| TRP | CA | B | 310 | 32.3 | 46.9 | 15.3 | 26 |
| TRP | CB | B | 310 | 31.9 | 46.0 | 16.4 | 27 |
| TRP | CG | B | 310 | 30.4 | 45.9 | 16.8 | 25 |
| TRP | CD2 | B | 310 | 29.4 | 44.9 | 16.4 | 24 |
| TRP | CE2 | B | 310 | 28.2 | 45.3 | 17.1 | 25 |
| TRP | CE3 | B | 310 | 29.5 | 43.8 | 15.6 | 27 |
| TRP | CD1 | B | 310 | 29.8 | 46.8 | 17.6 | 28 |
| TRP | NE1 | B | 310 | 28.5 | 46.4 | 17.8 | 29 |
| TRP | CZ2 | B | 310 | 27.1 | 44.5 | 16.9 | 26 |
| TRP | CZ3 | B | 310 | 28.3 | 43.0 | 15.4 | 26 |
| TRP | CH2 | B | 310 | 27.1 | 43.4 | 16.1 | 28 |
| TRP | C | B | 310 | 33.8 | 47.0 | 15.1 | 27 |
| TRP | O | B | 310 | 34.3 | 46.2 | 14.3 | 25 |
| GLN | N | B | 311 | 34.4 | 47.8 | 15.9 | 28 |
| GLN | CA | B | 311 | 35.9 | 47.9 | 15.8 | 36 |
| GLN | CB | B | 311 | 36.4 | 48.8 | 16.9 | 41 |
| GLN | CG | B | 311 | 38.0 | 48.9 | 16.9 | 49 |
| GLN | CD | B | 311 | 38.4 | 49.8 | 18.1 | 56 |
| GLN | OE1 | B | 311 | 39.2 | 49.4 | 18.9 | 55 |
| GLN | NE2 | B | 311 | 37.9 | 51.0 | 18.1 | 59 |
| GLN | C | B | 311 | 36.5 | 46.5 | 15.9 | 34 |
| GLN | O | B | 311 | 36.1 | 45.8 | 16.8 | 32 |
| ALA | N | B | 312 | 37.4 | 46.2 | 15.0 | 33 |
| ALA | CA | B | 312 | 38.0 | 44.9 | 15.0 | 34 |
| ALA | CB | B | 312 | 39.3 | 44.9 | 14.3 | 36 |
| ALA | C | B | 312 | 38.2 | 44.3 | 16.5 | 37 |
| ALA | O | B | 312 | 38.8 | 45.0 | 17.3 | 37 |
| GLY | N | B | 313 | 37.8 | 43.1 | 16.7 | 37 |
| GLY | CA | B | 313 | 38.0 | 42.5 | 18.0 | 37 |
| GLY | C | B | 313 | 37.1 | 42.9 | 19.1 | 36 |
| GLY | O | B | 313 | 37.3 | 42.5 | 20.3 | 38 |
| THR | N | B | 314 | 36.1 | 43.8 | 18.8 | 36 |
| THR | CA | B | 314 | 35.2 | 44.3 | 19.9 | 32 |
| THR | CB | B | 314 | 35.3 | 45.8 | 20.0 | 32 |
| THR | OG1 | B | 314 | 34.8 | 46.4 | 18.8 | 29 |
| THR | CG2 | B | 314 | 36.7 | 46.3 | 20.4 | 34 |
| THR | C | B | 314 | 33.8 | 43.8 | 19.8 | 31 |
| THR | O | B | 314 | 32.9 | 44.2 | 20.5 | 33 |
| THR | N | B | 315 | 33.5 | 42.9 | 18.8 | 29 |
| THR | CA | B | 315 | 32.2 | 42.4 | 18.6 | 29 |
| THR | CB | B | 315 | 32.1 | 41.2 | 17.7 | 30 |
| THR | OG1 | B | 315 | 32.6 | 41.5 | 16.4 | 27 |
| THR | CG2 | B | 315 | 30.7 | 40.6 | 17.6 | 30 |
| THR | C | B | 315 | 31.6 | 42.0 | 20.0 | 29 |
| THR | O | B | 315 | 32.2 | 41.2 | 20.7 | 28 |
| PRO | N | B | 316 | 30.5 | 42.6 | 20.4 | 27 |
| PRO | CD | B | 316 | 29.7 | 43.6 | 19.7 | 25 |
| PRO | CA | B | 316 | 29.9 | 42.3 | 21.7 | 22 |
| PRO | CB | B | 316 | 29.2 | 43.6 | 22.0 | 25 |
| PRO | CG | B | 316 | 28.5 | 43.8 | 20.7 | 30 |
| PRO | C | B | 316 | 29.0 | 41.1 | 21.6 | 23 |
| PRO | O | B | 316 | 27.7 | 41.2 | 21.7 | 21 |
| TRP | N | B | 317 | 29.6 | 39.9 | 21.6 | 23 |
| TRP | CA | B | 317 | 28.8 | 38.7 | 21.5 | 20 |
| TRP | CB | B | 317 | 29.7 | 37.5 | 21.4 | 21 |
| TRP | CG | B | 317 | 30.7 | 37.5 | 20.3 | 24 |
| TRP | CD2 | B | 317 | 30.5 | 37.3 | 18.9 | 19 |
| TRP | CE2 | B | 317 | 31.7 | 37.5 | 18.2 | 22 |
| TRP | CE3 | B | 317 | 29.4 | 37.1 | 18.1 | 18 |
| TRP | CD1 | B | 317 | 32.1 | 37.7 | 20.4 | 23 |
| TRP | NE1 | B | 317 | 32.7 | 37.7 | 19.2 | 23 |
| TRP | CZ2 | B | 317 | 31.9 | 37.3 | 16.9 | 19 |
| TRP | CZ3 | B | 317 | 29.5 | 37.0 | 16.7 | 18 |
| TRP | CH2 | B | 317 | 30.7 | 37.1 | 16.1 | 23 |
| TRP | C | B | 317 | 27.8 | 38.5 | 22.6 | 21 |
| TRP | O | B | 317 | 26.7 | 38.0 | 22.4 | 16 |
| ASN | N | B | 318 | 28.2 | 39.0 | 23.8 | 17 |
| ASN | CA | B | 318 | 27.4 | 38.8 | 25.0 | 16 |
| ASN | CB | B | 318 | 28.1 | 39.1 | 26.3 | 18 |
| ASN | CG | B | 318 | 28.4 | 40.6 | 26.3 | 20 |
| ASN | OD1 | B | 318 | 27.8 | 41.3 | 27.1 | 22 |
| ASN | ND2 | B | 318 | 29.4 | 41.0 | 25.5 | 21 |
| ASN | C | B | 318 | 26.0 | 39.5 | 25.0 | 17 |
| ASN | O | B | 318 | 25.1 | 39.1 | 25.7 | 20 |
| ILE | N | B | 319 | 25.8 | 40.5 | 24.1 | 20 |
| ILE | CA | B | 319 | 24.5 | 41.2 | 24.2 | 21 |
| ILE | CB | B | 319 | 24.6 | 42.6 | 23.7 | 26 |
| ILE | CG2 | B | 319 | 25.6 | 43.4 | 24.6 | 21 |
| ILE | CG1 | B | 319 | 25.1 | 42.7 | 22.2 | 29 |
| ILE | CD1 | B | 319 | 25.2 | 44.1 | 21.6 | 30 |
| ILE | C | B | 319 | 23.5 | 40.4 | 23.3 | 22 |
| ILE | O | B | 319 | 22.3 | 40.6 | 23.4 | 22 |
| PHE | N | B | 320 | 24.1 | 39.5 | 22.5 | 18 |
| PHE | CA | B | 320 | 23.2 | 38.6 | 21.7 | 23 |
| PHE | CB | B | 320 | 23.9 | 38.4 | 20.3 | 21 |
| PHE | CG | B | 320 | 24.0 | 39.6 | 19.4 | 22 |
| PHE | CD1 | B | 320 | 23.0 | 40.0 | 18.5 | 22 |
| PHE | CD2 | B | 320 | 25.1 | 40.5 | 19.5 | 19 |
| PHE | CE1 | B | 320 | 23.1 | 41.1 | 17.7 | 21 |
| PHE | CE2 | B | 320 | 25.2 | 41.6 | 18.8 | 19 |
| PHE | CZ | B | 320 | 24.2 | 41.9 | 17.9 | 20 |
| PHE | C | B | 320 | 22.9 | 37.3 | 22.3 | 20 |
| PHE | O | B | 320 | 23.8 | 36.7 | 22.9 | 22 |
| PRO | N | B | 321 | 21.6 | 36.9 | 22.3 | 21 |
| PRO | CD | B | 321 | 20.6 | 37.6 | 21.5 | 18 |
| PRO | CA | B | 321 | 21.1 | 35.7 | 22.9 | 19 |
| PRO | CB | B | 321 | 19.6 | 36.0 | 23.0 | 19 |
| PRO | CG | B | 321 | 19.4 | 36.5 | 21.6 | 20 |
| PRO | C | B | 321 | 21.4 | 34.4 | 22.1 | 21 |
| PRO | O | B | 321 | 21.6 | 34.4 | 20.9 | 23 |
| VAL | N | B | 322 | 21.5 | 33.3 | 22.9 | 20 |
| VAL | CA | B | 322 | 21.7 | 32.0 | 22.3 | 19 |
| VAL | CB | B | 322 | 22.3 | 31.0 | 23.3 | 17 |
| VAL | CG1 | B | 322 | 23.7 | 31.5 | 23.8 | 19 |
| VAL | CG2 | B | 322 | 21.3 | 30.7 | 24.5 | 18 |
| VAL | C | B | 322 | 20.3 | 31.5 | 21.8 | 21 |
| VAL | O | B | 322 | 19.3 | 31.9 | 22.3 | 18 |
| ILE | N | B | 323 | 20.3 | 30.6 | 20.8 | 23 |
| ILE | CA | B | 323 | 19.1 | 30.1 | 20.3 | 23 |
| ILE | CB | B | 323 | 18.9 | 30.4 | 18.8 | 25 |
| ILE | CG2 | B | 323 | 17.6 | 29.9 | 18.2 | 22 |
| ILE | CG1 | B | 323 | 19.0 | 32.0 | 18.6 | 28 |
| ILE | CD1 | B | 323 | 18.9 | 32.4 | 17.1 | 35 |
| ILE | C | B | 323 | 19.1 | 28.6 | 20.5 | 21 |
| ILE | O | B | 323 | 20.0 | 27.9 | 20.1 | 19 |
| SER | N | B | 324 | 18.0 | 28.1 | 21.1 | 19 |
| SER | CA | B | 324 | 17.9 | 26.7 | 21.4 | 21 |
| SER | CB | B | 324 | 17.8 | 26.4 | 22.9 | 20 |
| SER | OG | B | 324 | 18.9 | 26.8 | 23.6 | 23 |
| SER | C | B | 324 | 16.7 | 26.0 | 20.7 | 26 |
| SER | O | B | 324 | 15.6 | 26.4 | 20.8 | 30 |
| LEU | N | B | 325 | 17.0 | 24.9 | 19.9 | 21 |
| LEU | CA | B | 325 | 16.0 | 24.2 | 19.2 | 17 |
| LEU | CB | B | 325 | 16.3 | 24.0 | 17.7 | 21 |
| LEU | CG | B | 325 | 16.4 | 25.3 | 17.0 | 22 |
| LEU | CD1 | B | 325 | 16.7 | 25.0 | 15.5 | 22 |
| LEU | CD2 | B | 325 | 15.1 | 26.1 | 17.0 | 23 |
| LEU | C | B | 325 | 15.9 | 22.8 | 19.9 | 22 |
| LEU | O | B | 325 | 16.8 | 22.1 | 20.0 | 23 |
| TYR | N | B | 326 | 14.7 | 22.5 | 20.4 | 24 |
| TYR | CA | B | 326 | 14.4 | 21.2 | 21.0 | 22 |
| TYR | CB | B | 326 | 13.4 | 21.2 | 22.1 | 23 |
| TYR | CG | B | 326 | 13.9 | 21.9 | 23.4 | 20 |
| TYR | CD1 | B | 326 | 14.0 | 23.3 | 23.4 | 19 |
| TYR | CE1 | B | 326 | 14.4 | 23.9 | 24.6 | 19 |
| TYR | CD2 | B | 326 | 14.1 | 21.2 | 24.5 | 20 |
| TYR | CE2 | B | 326 | 14.5 | 21.8 | 25.7 | 22 |
| TYR | CZ | B | 326 | 14.7 | 23.2 | 25.7 | 21 |
| TYR | OH | B | 326 | 15.1 | 23.8 | 26.9 | 21 |
| TYR | C | B | 326 | 14.0 | 20.2 | 19.9 | 27 |
| TYR | O | B | 326 | 13.0 | 20.5 | 19.1 | 24 |
| LEU | N | B | 327 | 14.7 | 19.1 | 19.8 | 24 |
| LEU | CA | B | 327 | 14.4 | 18.1 | 18.7 | 25 |
| LEU | CB | B | 327 | 15.6 | 17.8 | 17.9 | 24 |
| LEU | CG | B | 327 | 16.2 | 19.0 | 17.2 | 20 |
| LEU | CD1 | B | 327 | 17.5 | 18.6 | 16.5 | 17 |
| LEU | CD2 | B | 327 | 15.2 | 19.6 | 16.2 | 20 |
| LEU | C | B | 327 | 13.9 | 16.8 | 19.4 | 27 |
| LEU | O | B | 327 | 14.3 | 16.4 | 20.4 | 31 |
| MET | N | B | 328 | 12.9 | 16.2 | 18.7 | 30 |
| MET | CA | B | 328 | 12.3 | 15.0 | 19.1 | 33 |
| MET | CB | B | 328 | 11.3 | 14.5 | 18.1 | 36 |
| MET | CG | B | 328 | 10.6 | 13.2 | 18.5 | 39 |
| MET | SD | B | 328 | 9.4 | 12.7 | 17.2 | 47 |
| MET | CE | B | 328 | 8.2 | 14.0 | 17.4 | 38 |
| MET | C | B | 328 | 13.4 | 13.9 | 19.3 | 35 |
| MET | O | B | 328 | 14.2 | 13.7 | 18.5 | 37 |
| GLY | N | B | 329 | 13.4 | 13.3 | 20.5 | 33 |
| GLY | CA | B | 329 | 14.4 | 12.3 | 20.8 | 33 |
| GLY | C | B | 329 | 14.1 | 10.9 | 20.3 | 33 |
| GLY | O | B | 329 | 13.0 | 10.7 | 19.7 | 32 |
| GLU | N | B | 330 | 14.9 | 9.9 | 20.5 | 36 |
| GLU | CA | B | 330 | 14.6 | 8.5 | 20.0 | 40 |
| GLU | CB | B | 330 | 15.9 | 7.7 | 19.8 | 38 |
| GLU | CG | B | 330 | 16.9 | 8.3 | 18.8 | 41 |
| GLU | CD | B | 330 | 18.1 | 7.4 | 18.6 | 44 |
| GLU | OE1 | B | 330 | 19.1 | 7.6 | 19.4 | 42 |
| GLU | OE2 | B | 330 | 18.0 | 6.4 | 17.8 | 36 |
| GLU | C | B | 330 | 13.7 | 7.8 | 21.0 | 38 |
| GLU | O | B | 330 | 13.1 | 6.8 | 20.7 | 41 |
| VAL | N | B | 331 | 13.6 | 8.3 | 22.2 | 40 |
| VAL | CA | B | 331 | 12.8 | 7.7 | 23.3 | 42 |
| VAL | CB | B | 331 | 13.5 | 7.6 | 24.6 | 44 |
| VAL | CG1 | B | 331 | 12.6 | 7.0 | 25.7 | 45 |
| VAL | CG2 | B | 331 | 14.9 | 6.9 | 24.5 | 45 |
| VAL | C | B | 331 | 11.5 | 8.4 | 23.4 | 44 |
| VAL | O | B | 331 | 11.4 | 9.6 | 23.5 | 44 |
| THR | N | B | 332 | 10.4 | 7.6 | 23.5 | 42 |
| THR | CA | B | 332 | 9.1 | 8.2 | 23.7 | 42 |
| THR | CB | B | 332 | 8.0 | 7.1 | 24.0 | 46 |
| THR | OG1 | B | 332 | 8.0 | 6.1 | 22.9 | 48 |
| THR | CG2 | B | 332 | 6.6 | 7.6 | 24.2 | 44 |
| THR | C | B | 332 | 9.0 | 9.2 | 24.8 | 38 |
| THR | O | B | 332 | 9.6 | 9.0 | 25.8 | 35 |
| ASN | N | B | 333 | 8.4 | 10.4 | 24.5 | 37 |
| ASN | CA | B | 333 | 8.3 | 11.4 | 25.5 | 41 |
| ASN | CB | B | 333 | 7.5 | 11.0 | 26.7 | 46 |
| ASN | CG | B | 333 | 6.1 | 10.6 | 26.4 | 52 |
| ASN | OD1 | B | 333 | 5.6 | 10.8 | 25.3 | 52 |
| ASN | ND2 | B | 333 | 5.4 | 10.0 | 27.4 | 54 |
| ASN | C | B | 333 | 9.6 | 12.1 | 25.9 | 41 |
| ASN | O | B | 333 | 9.7 | 12.8 | 26.9 | 43 |
| GLN | N | B | 334 | 10.6 | 11.9 | 25.1 | 40 |
| GLN | CA | B | 334 | 11.9 | 12.4 | 25.4 | 40 |
| GLN | CB | B | 334 | 12.9 | 11.3 | 25.8 | 43 |
| GLN | CG | B | 334 | 14.3 | 11.7 | 26.2 | 45 |
| GLN | CD | B | 334 | 15.1 | 10.5 | 26.6 | 48 |
| GLN | OE1 | B | 334 | 16.1 | 10.2 | 26.0 | 52 |
| GLN | NE2 | B | 334 | 14.6 | 9.8 | 27.6 | 45 |
| GLN | C | B | 334 | 12.5 | 13.3 | 24.3 | 39 |
| GLN | O | B | 334 | 12.5 | 12.9 | 23.1 | 38 |
| SER | N | B | 335 | 13.0 | 14.4 | 24.6 | 36 |
| SER | CA | B | 335 | 13.6 | 15.4 | 23.6 | 35 |
| SER | CB | B | 335 | 12.7 | 16.5 | 23.3 | 36 |
| SER | OG | B | 335 | 12.5 | 17.3 | 24.5 | 41 |
| SER | C | B | 335 | 15.0 | 15.9 | 24.2 | 30 |
| SER | O | B | 335 | 15.3 | 15.7 | 25.3 | 28 |
| PHE | N | B | 336 | 15.8 | 16.5 | 23.3 | 28 |
| PHE | CA | B | 336 | 17.1 | 17.0 | 23.6 | 28 |
| PHE | CB | B | 336 | 18.2 | 16.1 | 23.1 | 26 |
| PHE | CG | B | 336 | 18.3 | 15.9 | 21.6 | 27 |
| PHE | CD1 | B | 336 | 19.1 | 16.8 | 20.8 | 27 |
| PHE | CD2 | B | 336 | 17.6 | 14.9 | 21.0 | 26 |
| PHE | CE1 | B | 336 | 19.2 | 16.6 | 19.5 | 24 |
| PHE | CE2 | B | 336 | 17.7 | 14.7 | 19.6 | 27 |
| PHE | CZ | B | 336 | 18.5 | 15.5 | 18.9 | 27 |
| PHE | C | B | 336 | 17.1 | 18.4 | 22.9 | 26 |
| PHE | O | B | 336 | 16.3 | 18.7 | 22.0 | 26 |
| ARG | N | B | 337 | 18.0 | 19.3 | 23.3 | 22 |
| ARG | CA | B | 337 | 18.1 | 20.6 | 22.6 | 25 |
| ARG | CB | B | 337 | 17.7 | 21.7 | 23.5 | 23 |
| ARG | CG | B | 337 | 18.6 | 21.9 | 24.7 | 26 |
| ARG | CD | B | 337 | 18.1 | 23.1 | 25.6 | 26 |
| ARG | NE | B | 337 | 19.0 | 23.2 | 26.7 | 28 |
| ARG | CZ | B | 337 | 18.7 | 23.6 | 27.9 | 28 |
| ARG | NH1 | B | 337 | 17.4 | 23.9 | 28.2 | 27 |
| ARG | NH2 | B | 337 | 19.6 | 23.7 | 28.9 | 21 |
| ARG | C | B | 337 | 19.5 | 20.9 | 22.1 | 24 |
| ARG | O | B | 337 | 20.5 | 20.5 | 22.6 | 26 |
| ILE | N | B | 338 | 19.5 | 21.6 | 20.9 | 26 |
| ILE | CA | B | 338 | 20.8 | 22.0 | 20.3 | 24 |
| ILE | CB | B | 338 | 20.9 | 21.6 | 18.9 | 23 |
| ILE | CG2 | B | 338 | 21.0 | 20.1 | 18.8 | 19 |
| ILE | CG1 | B | 338 | 19.8 | 22.1 | 18.0 | 22 |
| ILE | CD1 | B | 338 | 19.9 | 21.7 | 16.6 | 23 |
| ILE | C | B | 338 | 20.8 | 23.6 | 20.5 | 23 |
| ILE | O | B | 338 | 19.8 | 24.2 | 20.3 | 20 |
| THR | N | B | 339 | 21.9 | 24.1 | 20.9 | 20 |
| THR | CA | B | 339 | 22.0 | 25.5 | 21.1 | 16 |
| THR | CB | B | 339 | 22.1 | 25.8 | 22.6 | 19 |
| THR | OG1 | B | 339 | 21.0 | 25.3 | 23.3 | 21 |
| THR | CG2 | B | 339 | 22.2 | 27.3 | 22.9 | 18 |
| THR | C | B | 339 | 23.2 | 26.2 | 20.4 | 19 |
| THR | O | B | 339 | 24.3 | 25.7 | 20.5 | 18 |
| ILE | N | B | 340 | 22.8 | 27.2 | 19.6 | 19 |
| ILE | CA | B | 340 | 23.8 | 27.9 | 18.8 | 19 |
| ILE | CB | B | 340 | 23.5 | 28.1 | 17.3 | 20 |
| ILE | CG2 | B | 340 | 23.4 | 26.7 | 16.7 | 14 |
| ILE | CG1 | B | 340 | 22.3 | 28.9 | 17.1 | 18 |
| ILE | CD1 | B | 340 | 21.9 | 29.2 | 15.7 | 19 |
| ILE | C | B | 340 | 24.1 | 29.3 | 19.4 | 23 |
| ILE | O | B | 340 | 23.2 | 29.8 | 20.1 | 22 |
| LEU | N | B | 341 | 25.2 | 29.8 | 19.1 | 20 |
| LEU | CA | B | 341 | 25.7 | 31.1 | 19.6 | 19 |
| LEU | CB | B | 341 | 27.1 | 31.1 | 20.1 | 16 |
| LEU | CG | B | 341 | 27.4 | 30.1 | 21.2 | 18 |
| LEU | CD1 | B | 341 | 28.9 | 30.1 | 21.5 | 19 |
| LEU | CD2 | B | 341 | 26.6 | 30.3 | 22.4 | 16 |
| LEU | C | B | 341 | 25.5 | 32.2 | 18.5 | 16 |
| LEU | O | B | 341 | 25.4 | 31.9 | 17.3 | 14 |
| PRO | N | B | 342 | 25.6 | 33.5 | 18.9 | 15 |
| PRO | CD | B | 342 | 25.9 | 34.2 | 20.2 | 16 |
| PRO | CA | B | 342 | 25.5 | 34.5 | 17.9 | 16 |
| PRO | CB | B | 342 | 25.3 | 35.8 | 18.6 | 19 |
| PRO | CG | B | 342 | 26.3 | 35.6 | 19.7 | 16 |
| PRO | C | B | 342 | 26.7 | 34.4 | 16.9 | 17 |
| PRO | O | B | 342 | 26.7 | 34.9 | 15.8 | 16 |
| GLN | N | B | 343 | 27.8 | 33.7 | 17.4 | 18 |
| GLN | CA | B | 343 | 28.9 | 33.5 | 16.5 | 17 |
| GLN | CB | B | 343 | 30.2 | 32.8 | 17.1 | 17 |
| GLN | CG | B | 343 | 31.0 | 33.6 | 18.2 | 18 |
| GLN | CD | B | 343 | 30.3 | 33.8 | 19.5 | 20 |
| GLN | OE1 | B | 343 | 29.1 | 33.6 | 19.6 | 24 |
| GLN | NE2 | B | 343 | 31.1 | 34.3 | 20.4 | 21 |
| GLN | C | B | 343 | 28.5 | 32.6 | 15.3 | 22 |
| GLN | O | B | 343 | 29.1 | 32.6 | 14.2 | 23 |
| GLN | N | B | 344 | 27.4 | 31.8 | 15.5 | 23 |
| GLN | CA | B | 344 | 26.8 | 31.0 | 14.4 | 20 |
| GLN | CB | B | 344 | 26.2 | 29.7 | 14.8 | 16 |
| GLN | CG | B | 344 | 27.1 | 28.5 | 15.3 | 17 |
| GLN | CD | B | 344 | 27.9 | 28.8 | 16.6 | 18 |
| GLN | OE1 | B | 344 | 29.1 | 28.9 | 16.6 | 23 |
| GLN | NE2 | B | 344 | 27.1 | 29.0 | 17.7 | 16 |
| GLN | C | B | 344 | 25.8 | 31.8 | 13.5 | 23 |
| GLN | O | B | 344 | 25.7 | 31.5 | 12.3 | 23 |
| TYR | N | B | 345 | 25.0 | 32.7 | 14.1 | 21 |
| TYR | CA | B | 345 | 24.0 | 33.3 | 13.3 | 21 |
| TYR | CB | B | 345 | 22.6 | 33.3 | 14.0 | 18 |
| TYR | CG | B | 345 | 22.5 | 34.1 | 15.3 | 19 |
| TYR | CD1 | B | 345 | 22.4 | 35.5 | 15.3 | 18 |
| TYR | CE1 | B | 345 | 22.1 | 36.1 | 16.5 | 20 |
| TYR | CD2 | B | 345 | 22.4 | 33.4 | 16.5 | 19 |
| TYR | CE2 | B | 345 | 22.2 | 34.1 | 17.7 | 20 |
| TYR | CZ | B | 345 | 22.0 | 35.4 | 17.7 | 21 |
| TYR | OH | B | 345 | 21.8 | 36.1 | 18.9 | 21 |
| TYR | C | B | 345 | 24.4 | 34.7 | 12.8 | 22 |
| TYR | O | B | 345 | 23.6 | 35.3 | 12.0 | 22 |
| LEU | N | B | 346 | 25.5 | 35.2 | 13.2 | 23 |
| LEU | CA | B | 346 | 26.1 | 36.5 | 12.7 | 22 |
| LEU | CB | B | 346 | 26.6 | 37.4 | 13.8 | 22 |
| LEU | CG | B | 346 | 25.5 | 37.8 | 14.8 | 24 |
| LEU | CD1 | B | 346 | 26.1 | 38.7 | 15.9 | 26 |
| LEU | CD2 | B | 346 | 24.3 | 38.6 | 14.1 | 23 |
| LEU | C | B | 346 | 27.2 | 36.1 | 11.8 | 23 |
| LEU | O | B | 346 | 28.3 | 35.8 | 12.3 | 26 |
| ARG | N | B | 347 | 27.0 | 36.1 | 10.5 | 23 |
| ARG | CA | B | 347 | 28.0 | 35.7 | 9.5 | 24 |
| ARG | CB | B | 347 | 27.3 | 35.1 | 8.3 | 21 |
| ARG | CG | B | 347 | 28.3 | 34.6 | 7.2 | 26 |
| ARG | CD | B | 347 | 27.5 | 33.9 | 6.0 | 30 |
| ARG | NE | B | 347 | 26.5 | 34.8 | 5.3 | 32 |
| ARG | CZ | B | 347 | 26.8 | 35.6 | 4.3 | 37 |
| ARG | NH1 | B | 347 | 28.1 | 35.7 | 3.9 | 39 |
| ARG | NH2 | B | 347 | 25.9 | 36.2 | 3.6 | 40 |
| ARG | C | B | 347 | 29.1 | 36.7 | 9.1 | 28 |
| ARG | O | B | 347 | 28.8 | 37.8 | 8.7 | 24 |
| PRO | N | B | 348 | 30.3 | 36.4 | 9.4 | 31 |
| PRO | CD | B | 348 | 30.8 | 35.3 | 10.3 | 31 |
| PRO | CA | B | 348 | 31.5 | 37.2 | 9.0 | 31 |
| PRO | CB | B | 348 | 32.7 | 36.3 | 9.4 | 33 |
| PRO | CG | B | 348 | 32.2 | 35.8 | 10.7 | 34 |
| PRO | C | B | 348 | 31.5 | 37.6 | 7.6 | 33 |
| PRO | O | B | 348 | 31.5 | 36.7 | 6.7 | 31 |
| VAL | N | B | 349 | 31.5 | 38.9 | 7.3 | 34 |
| VAL | CA | B | 349 | 31.5 | 39.4 | 5.9 | 40 |
| VAL | CB | B | 349 | 30.3 | 40.2 | 5.5 | 39 |
| VAL | CG1 | B | 349 | 30.4 | 40.7 | 4.1 | 41 |
| VAL | CG2 | B | 349 | 29.0 | 39.3 | 5.6 | 37 |
| VAL | C | B | 349 | 32.8 | 40.2 | 5.7 | 47 |
| VAL | O | B | 349 | 32.9 | 41.3 | 6.2 | 45 |
| GLU | N | B | 350 | 33.7 | 39.7 | 4.9 | 57 |
| GLU | CA | B | 350 | 34.9 | 40.4 | 4.5 | 68 |
| GLU | CB | B | 350 | 36.1 | 39.4 | 4.6 | 71 |
| GLU | CG | B | 350 | 37.5 | 40.0 | 4.3 | 76 |
| GLU | CD | B | 350 | 37.8 | 41.2 | 5.2 | 78 |
| GLU | OE1 | B | 350 | 37.9 | 42.3 | 4.7 | 78 |
| GLU | OE2 | B | 350 | 37.9 | 40.9 | 6.4 | 79 |
| GLU | C | B | 350 | 34.7 | 40.9 | 3.1 | 75 |
| GLU | O | B | 350 | 34.6 | 40.1 | 2.2 | 78 |
| ASP | N | B | 351 | 34.8 | 42.2 | 2.9 | 81 |
| ASP | CA | B | 351 | 34.6 | 42.7 | 1.6 | 88 |
| ASP | CB | B | 351 | 34.2 | 44.2 | 1.7 | 92 |
| ASP | CG | B | 351 | 34.0 | 44.8 | 0.3 | 94 |
| ASP | OD1 | B | 351 | 32.8 | 45.2 | 0.0 | 96 |
| ASP | OD2 | B | 351 | 34.9 | 44.9 | −0.5 | 96 |
| ASP | C | B | 351 | 35.9 | 42.6 | 0.7 | 89 |
| ASP | O | B | 351 | 37.0 | 42.6 | 1.2 | 90 |
| SER | N | B | 355 | 39.0 | 45.0 | 7.3 | 74 |
| SER | CA | B | 355 | 39.9 | 46.1 | 7.6 | 74 |
| SER | CB | B | 355 | 39.6 | 47.4 | 6.9 | 75 |
| SER | OG | B | 355 | 38.3 | 47.8 | 7.3 | 77 |
| SER | C | B | 355 | 40.1 | 46.3 | 9.1 | 72 |
| SER | O | B | 355 | 40.5 | 45.4 | 9.8 | 73 |
| GLN | N | B | 356 | 39.7 | 47.5 | 9.6 | 69 |
| GLN | CA | B | 356 | 39.8 | 47.8 | 11.0 | 65 |
| GLN | CB | B | 356 | 40.3 | 49.2 | 11.2 | 70 |
| GLN | CG | B | 356 | 40.5 | 49.6 | 12.7 | 77 |
| GLN | CD | B | 356 | 41.5 | 48.7 | 13.3 | 79 |
| GLN | OE1 | B | 356 | 42.6 | 49.2 | 13.8 | 81 |
| GLN | NE2 | B | 356 | 41.2 | 47.4 | 13.4 | 80 |
| GLN | C | B | 356 | 38.5 | 47.5 | 11.7 | 58 |
| GLN | O | B | 356 | 38.4 | 47.7 | 12.9 | 56 |
| ASP | N | B | 357 | 37.5 | 47.0 | 11.0 | 52 |
| ASP | CA | B | 357 | 36.2 | 46.7 | 11.5 | 45 |
| ASP | CB | B | 357 | 35.1 | 47.7 | 11.0 | 48 |
| ASP | CG | B | 357 | 35.3 | 49.1 | 11.4 | 52 |
| ASP | OD1 | B | 357 | 36.2 | 49.4 | 12.2 | 54 |
| ASP | OD2 | B | 357 | 34.6 | 50.0 | 10.9 | 53 |
| ASP | C | B | 357 | 35.8 | 45.3 | 11.2 | 43 |
| ASP | O | B | 357 | 36.0 | 44.8 | 10.1 | 42 |
| ASP | N | B | 358 | 35.1 | 44.6 | 12.1 | 39 |
| ASP | CA | B | 358 | 34.6 | 43.3 | 11.9 | 32 |
| ASP | CB | B | 358 | 34.8 | 42.4 | 13.1 | 33 |
| ASP | CG | B | 358 | 36.3 | 42.1 | 13.4 | 36 |
| ASP | OD1 | B | 358 | 36.6 | 42.1 | 14.6 | 37 |
| ASP | OD2 | B | 358 | 37.1 | 42.0 | 12.5 | 38 |
| ASP | C | B | 358 | 33.1 | 43.6 | 11.6 | 26 |
| ASP | O | B | 358 | 32.4 | 44.1 | 12.4 | 24 |
| CYS | N | B | 359 | 32.7 | 43.2 | 10.4 | 22 |
| CYS | CA | B | 359 | 31.3 | 43.4 | 10.0 | 24 |
| CYS | C | B | 359 | 30.6 | 42.1 | 9.8 | 29 |
| CYS | O | B | 359 | 31.2 | 41.0 | 9.5 | 26 |
| CYS | CB | B | 359 | 31.3 | 44.2 | 8.7 | 28 |
| CYS | SG | B | 359 | 32.1 | 45.8 | 8.7 | 33 |
| TYR | N | B | 360 | 29.3 | 42.1 | 10.1 | 27 |
| TYR | CA | B | 360 | 28.5 | 40.8 | 10.1 | 27 |
| TYR | CB | B | 360 | 28.3 | 40.3 | 11.5 | 27 |
| TYR | CG | B | 360 | 29.6 | 40.1 | 12.3 | 26 |
| TYR | CD1 | B | 360 | 30.2 | 41.2 | 12.9 | 25 |
| TYR | CE1 | B | 360 | 31.4 | 41.1 | 13.6 | 26 |
| TYR | CD2 | B | 360 | 30.2 | 38.8 | 12.4 | 24 |
| TYR | CE2 | B | 360 | 31.4 | 38.7 | 13.1 | 26 |
| TYR | CZ | B | 360 | 32.0 | 39.8 | 13.7 | 27 |
| TYR | OH | B | 360 | 33.1 | 39.7 | 14.4 | 29 |
| TYR | C | B | 360 | 27.1 | 41.0 | 9.5 | 29 |
| TYR | O | B | 360 | 26.5 | 42.0 | 9.5 | 27 |
| LYS | N | B | 361 | 26.7 | 39.9 | 8.8 | 25 |
| LYS | CA | B | 361 | 25.3 | 39.9 | 8.3 | 27 |
| LYS | CB | B | 361 | 25.3 | 39.5 | 6.8 | 31 |
| LYS | CG | B | 361 | 23.9 | 39.4 | 6.2 | 39 |
| LYS | CD | B | 361 | 23.9 | 39.1 | 4.7 | 45 |
| LYS | CE | B | 361 | 24.7 | 40.1 | 3.9 | 50 |
| LYS | NZ | B | 361 | 24.7 | 39.8 | 2.5 | 56 |
| LYS | C | B | 361 | 24.5 | 38.9 | 9.1 | 28 |
| LYS | O | B | 361 | 24.9 | 37.9 | 9.5 | 30 |
| PHE | N | B | 362 | 23.3 | 39.4 | 9.4 | 27 |
| PHE | CA | B | 362 | 22.3 | 38.6 | 10.2 | 22 |
| PHE | CB | B | 362 | 21.2 | 39.5 | 10.6 | 21 |
| PHE | CG | B | 362 | 20.1 | 38.8 | 11.5 | 22 |
| PHE | CD1 | B | 362 | 20.5 | 38.3 | 12.7 | 20 |
| PHE | CD2 | B | 362 | 18.8 | 38.6 | 11.0 | 23 |
| PHE | CE1 | B | 362 | 19.5 | 37.6 | 13.5 | 21 |
| PHE | CE2 | B | 362 | 17.9 | 38.0 | 11.8 | 22 |
| PHE | CZ | B | 362 | 18.2 | 37.5 | 13.1 | 22 |
| PHE | C | B | 362 | 21.9 | 37.4 | 9.3 | 23 |
| PHE | O | B | 362 | 21.2 | 37.6 | 8.3 | 25 |
| ALA | N | B | 363 | 22.3 | 36.2 | 9.7 | 23 |
| ALA | CA | B | 363 | 22.1 | 35.0 | 8.9 | 20 |
| ALA | CB | B | 363 | 23.3 | 34.1 | 8.9 | 21 |
| ALA | C | B | 363 | 20.8 | 34.2 | 9.2 | 23 |
| ALA | O | B | 363 | 20.7 | 33.0 | 9.0 | 25 |
| ILE | N | B | 364 | 19.8 | 34.9 | 9.8 | 21 |
| ILE | CA | B | 364 | 18.5 | 34.2 | 10.1 | 23 |
| ILE | CB | B | 364 | 18.1 | 34.3 | 11.6 | 22 |
| ILE | CG2 | B | 364 | 16.8 | 33.7 | 11.8 | 19 |
| ILE | CG1 | B | 364 | 19.2 | 33.7 | 12.5 | 19 |
| ILE | CD1 | B | 364 | 18.9 | 33.8 | 14.0 | 21 |
| ILE | C | B | 364 | 17.5 | 34.9 | 9.2 | 25 |
| ILE | O | B | 364 | 17.4 | 36.1 | 9.2 | 24 |
| SER | N | B | 365 | 16.8 | 34.1 | 8.4 | 24 |
| SER | CA | B | 365 | 15.8 | 34.7 | 7.5 | 26 |
| SER | CB | B | 365 | 16.4 | 34.9 | 6.1 | 24 |
| SER | OG | B | 365 | 16.9 | 33.7 | 5.6 | 32 |
| SER | C | B | 365 | 14.5 | 34.0 | 7.4 | 27 |
| SER | O | B | 365 | 14.3 | 32.8 | 7.7 | 30 |
| GLN | N | B | 366 | 13.5 | 34.7 | 6.9 | 28 |
| GLN | CA | B | 366 | 12.1 | 34.2 | 6.6 | 31 |
| GLN | CB | B | 366 | 11.2 | 35.4 | 6.3 | 37 |
| GLN | CG | B | 366 | 9.8 | 35.0 | 6.1 | 45 |
| GLN | CD | B | 366 | 9.0 | 36.3 | 5.8 | 51 |
| GLN | OE1 | B | 366 | 8.3 | 36.4 | 4.7 | 54 |
| GLN | NE2 | B | 366 | 9.1 | 37.3 | 6.6 | 50 |
| GLN | C | B | 366 | 12.0 | 33.1 | 5.6 | 28 |
| GLN | O | B | 366 | 12.7 | 33.2 | 4.6 | 27 |
| SER | N | B | 367 | 11.2 | 32.2 | 5.8 | 27 |
| SER | CA | B | 367 | 11.0 | 31.1 | 4.9 | 27 |
| SER | CB | B | 367 | 11.5 | 29.7 | 5.4 | 26 |
| SER | OG | B | 367 | 11.3 | 28.7 | 4.5 | 28 |
| SER | C | B | 367 | 9.5 | 30.9 | 4.6 | 29 |
| SER | O | B | 367 | 8.6 | 31.1 | 5.5 | 27 |
| SER | N | B | 368 | 9.2 | 30.5 | 3.4 | 29 |
| SER | CA | B | 368 | 7.8 | 30.2 | 3.0 | 36 |
| SER | CB | B | 368 | 7.4 | 31.0 | 1.7 | 37 |
| SER | OG | B | 368 | 8.2 | 30.7 | 0.6 | 40 |
| SER | C | B | 368 | 7.6 | 28.7 | 2.7 | 36 |
| SER | O | B | 368 | 6.5 | 28.3 | 2.3 | 39 |
| THR | N | B | 369 | 8.7 | 28.0 | 3.0 | 36 |
| THR | CA | B | 369 | 8.7 | 26.5 | 2.7 | 31 |
| THR | CB | B | 369 | 9.4 | 26.1 | 1.5 | 33 |
| THR | OG1 | B | 369 | 10.8 | 26.5 | 1.6 | 34 |
| THR | CG2 | B | 369 | 8.8 | 26.7 | 0.2 | 33 |
| THR | C | B | 369 | 9.1 | 25.7 | 3.9 | 32 |
| THR | O | B | 369 | 9.6 | 24.6 | 3.8 | 32 |
| GLY | N | B | 370 | 8.9 | 26.2 | 5.1 | 29 |
| GLY | CA | B | 370 | 9.3 | 25.5 | 6.3 | 28 |
| GLY | C | B | 370 | 10.6 | 25.9 | 6.9 | 29 |
| GLY | O | B | 370 | 11.3 | 26.8 | 6.4 | 28 |
| THR | N | B | 371 | 11.0 | 25.2 | 8.0 | 27 |
| THR | CA | B | 371 | 12.3 | 25.5 | 8.7 | 23 |
| THR | CB | B | 371 | 12.3 | 25.1 | 10.1 | 23 |
| THR | OG1 | B | 371 | 11.4 | 25.9 | 10.9 | 24 |
| THR | CG2 | B | 371 | 13.7 | 25.3 | 10.7 | 17 |
| THR | C | B | 371 | 13.5 | 24.9 | 7.9 | 25 |
| THR | O | B | 371 | 13.4 | 23.7 | 7.6 | 26 |
| VAL | N | B | 372 | 14.5 | 25.7 | 7.7 | 20 |
| VAL | CA | B | 372 | 15.7 | 25.2 | 7.1 | 20 |
| VAL | CB | B | 372 | 16.0 | 25.8 | 5.7 | 21 |
| VAL | CG1 | B | 372 | 17.3 | 25.2 | 5.1 | 16 |
| VAL | CG2 | B | 372 | 14.8 | 25.6 | 4.7 | 15 |
| VAL | C | B | 372 | 16.9 | 25.3 | 8.0 | 22 |
| VAL | O | B | 372 | 17.4 | 26.4 | 8.2 | 18 |
| MET | N | B | 373 | 17.4 | 24.2 | 8.5 | 21 |
| MET | CA | B | 373 | 18.5 | 24.3 | 9.4 | 20 |
| MET | CB | B | 373 | 18.5 | 23.1 | 10.4 | 23 |
| MET | CG | B | 373 | 17.3 | 23.1 | 11.2 | 22 |
| MET | SD | B | 373 | 17.2 | 21.6 | 12.4 | 31 |
| MET | CE | B | 373 | 18.9 | 21.5 | 12.8 | 25 |
| MET | C | B | 373 | 19.7 | 24.2 | 8.5 | 21 |
| MET | O | B | 373 | 20.2 | 23.1 | 8.1 | 18 |
| GLY | N | B | 374 | 20.2 | 25.4 | 8.1 | 18 |
| GLY | CA | B | 374 | 21.4 | 25.5 | 7.2 | 20 |
| GLY | C | B | 374 | 22.7 | 25.6 | 7.9 | 18 |
| GLY | O | B | 374 | 22.8 | 25.1 | 9.0 | 20 |
| ALA | N | B | 375 | 23.7 | 26.1 | 7.2 | 19 |
| ALA | CA | B | 375 | 25.0 | 26.2 | 7.8 | 21 |
| ALA | CB | B | 375 | 26.0 | 26.9 | 6.8 | 15 |
| ALA | C | B | 375 | 25.0 | 27.0 | 9.1 | 22 |
| ALA | O | B | 375 | 25.9 | 26.7 | 9.9 | 24 |
| VAL | N | B | 376 | 24.1 | 27.8 | 9.4 | 22 |
| VAL | CA | B | 376 | 24.1 | 28.5 | 10.7 | 18 |
| VAL | CB | B | 376 | 22.9 | 29.6 | 10.7 | 19 |
| VAL | CG1 | B | 376 | 22.9 | 30.2 | 12.1 | 18 |
| VAL | CG2 | B | 376 | 23.2 | 30.7 | 9.7 | 22 |
| VAL | C | B | 376 | 23.9 | 27.6 | 11.8 | 22 |
| VAL | O | B | 376 | 24.5 | 27.8 | 12.9 | 20 |
| ILE | N | B | 377 | 23.2 | 26.5 | 11.6 | 20 |
| ILE | CA | B | 377 | 23.0 | 25.5 | 12.6 | 22 |
| ILE | CB | B | 377 | 21.6 | 24.8 | 12.4 | 25 |
| ILE | CG2 | B | 377 | 21.5 | 23.5 | 13.3 | 21 |
| ILE | CG1 | B | 377 | 20.5 | 25.7 | 12.7 | 25 |
| ILE | CD1 | B | 377 | 20.4 | 26.3 | 14.1 | 29 |
| ILE | C | B | 377 | 24.1 | 24.4 | 12.5 | 21 |
| ILE | O | B | 377 | 24.7 | 24.1 | 13.5 | 23 |
| MET | N | B | 378 | 24.5 | 24.0 | 11.3 | 20 |
| MET | CA | B | 378 | 25.5 | 23.0 | 11.2 | 24 |
| MET | CB | B | 378 | 25.5 | 22.4 | 9.7 | 22 |
| MET | CG | B | 378 | 24.1 | 21.7 | 9.4 | 24 |
| MET | SD | B | 378 | 24.1 | 21.0 | 7.8 | 25 |
| MET | CE | B | 378 | 25.1 | 19.5 | 8.0 | 18 |
| MET | C | B | 378 | 26.9 | 23.4 | 11.6 | 24 |
| MET | O | B | 378 | 27.7 | 22.5 | 12.0 | 24 |
| GLU | N | B | 379 | 27.2 | 24.6 | 11.5 | 23 |
| GLU | CA | B | 379 | 28.6 | 25.1 | 11.9 | 22 |
| GLU | CB | B | 379 | 28.9 | 26.4 | 11.3 | 18 |
| GLU | CG | B | 379 | 28.8 | 26.4 | 9.7 | 24 |
| GLU | CD | B | 379 | 29.1 | 27.8 | 9.2 | 24 |
| GLU | OE1 | B | 379 | 29.5 | 27.9 | 8.1 | 23 |
| GLU | OE2 | B | 379 | 28.8 | 28.8 | 9.9 | 27 |
| GLU | C | B | 379 | 28.9 | 25.0 | 13.4 | 20 |
| GLU | O | B | 379 | 30.0 | 25.3 | 13.8 | 23 |
| GLY | N | B | 380 | 27.8 | 24.7 | 14.1 | 19 |
| GLY | CA | B | 380 | 28.1 | 24.6 | 15.6 | 20 |
| GLY | C | B | 380 | 28.0 | 23.1 | 16.0 | 18 |
| GLY | O | B | 380 | 28.3 | 22.8 | 17.1 | 19 |
| PHE | N | B | 381 | 27.7 | 22.2 | 15.0 | 17 |
| PHE | CA | B | 381 | 27.6 | 20.8 | 15.4 | 19 |
| PHE | CB | B | 381 | 26.1 | 20.5 | 15.6 | 17 |
| PHE | CG | B | 381 | 25.4 | 21.4 | 16.5 | 24 |
| PHE | CD1 | B | 381 | 24.6 | 22.4 | 16.0 | 22 |
| PHE | CD2 | B | 381 | 25.6 | 21.3 | 17.9 | 24 |
| PHE | CE1 | B | 381 | 24.0 | 23.3 | 16.9 | 25 |
| PHE | CE2 | B | 381 | 25.0 | 22.2 | 18.8 | 23 |
| PHE | CZ | B | 381 | 24.2 | 23.2 | 18.3 | 22 |
| PHE | C | B | 381 | 28.2 | 19.9 | 14.4 | 19 |
| PHE | O | B | 381 | 28.2 | 20.1 | 13.2 | 21 |
| TYR | N | B | 382 | 28.6 | 18.7 | 14.9 | 21 |
| TYR | CA | B | 382 | 29.1 | 17.6 | 14.0 | 18 |
| TYR | CB | B | 382 | 30.0 | 16.7 | 14.8 | 14 |
| TYR | CG | B | 382 | 30.5 | 15.5 | 14.0 | 21 |
| TYR | CD1 | B | 382 | 30.9 | 15.7 | 12.6 | 20 |
| TYR | CE1 | B | 382 | 31.4 | 14.6 | 11.9 | 21 |
| TYR | CD2 | B | 382 | 30.6 | 14.3 | 14.5 | 20 |
| TYR | CE2 | B | 382 | 31.1 | 13.2 | 13.8 | 25 |
| TYR | CZ | B | 382 | 31.5 | 13.3 | 12.5 | 19 |
| TYR | OH | B | 382 | 32.0 | 12.3 | 11.8 | 23 |
| TYR | C | B | 382 | 27.7 | 17.0 | 13.8 | 17 |
| TYR | O | B | 382 | 27.0 | 16.6 | 14.7 | 19 |
| VAL | N | B | 383 | 27.3 | 16.8 | 12.5 | 16 |
| VAL | CA | B | 383 | 26.0 | 16.2 | 12.2 | 17 |
| VAL | CB | B | 383 | 25.2 | 17.2 | 11.3 | 16 |
| VAL | CG1 | B | 383 | 23.8 | 16.6 | 11.0 | 13 |
| VAL | CG2 | B | 383 | 25.1 | 18.6 | 12.0 | 12 |
| VAL | C | B | 383 | 26.1 | 14.8 | 11.5 | 17 |
| VAL | O | B | 383 | 26.8 | 14.7 | 10.5 | 20 |
| VAL | N | B | 384 | 25.5 | 13.9 | 12.1 | 19 |
| VAL | CA | B | 384 | 25.5 | 12.5 | 11.6 | 20 |
| VAL | CB | B | 384 | 25.7 | 11.4 | 12.6 | 20 |
| VAL | CG1 | B | 384 | 25.8 | 10.0 | 12.0 | 16 |
| VAL | CG2 | B | 384 | 27.0 | 11.8 | 13.5 | 13 |
| VAL | C | B | 384 | 24.2 | 12.1 | 10.8 | 21 |
| VAL | O | B | 384 | 23.1 | 12.0 | 11.4 | 20 |
| PHE | N | B | 385 | 24.3 | 11.9 | 9.5 | 18 |
| PHE | CA | B | 385 | 23.2 | 11.5 | 8.7 | 19 |
| PHE | CB | B | 385 | 23.2 | 12.1 | 7.3 | 14 |
| PHE | CG | B | 385 | 23.1 | 13.6 | 7.3 | 19 |
| PHE | CD1 | B | 385 | 21.9 | 14.2 | 6.9 | 10 |
| PHE | CD2 | B | 385 | 24.2 | 14.3 | 7.7 | 16 |
| PHE | CE1 | B | 385 | 21.8 | 15.6 | 7.0 | 16 |
| PHE | CE2 | B | 385 | 24.1 | 15.7 | 7.7 | 15 |
| PHE | CZ | B | 385 | 22.9 | 16.4 | 7.4 | 17 |
| PHE | C | B | 385 | 23.2 | 10.0 | 8.8 | 17 |
| PHE | O | B | 385 | 23.8 | 9.4 | 7.9 | 17 |
| ASP | N | B | 386 | 22.5 | 9.4 | 9.7 | 23 |
| ASP | CA | B | 386 | 22.5 | 8.0 | 9.9 | 25 |
| ASP | CB | B | 386 | 22.5 | 7.6 | 11.4 | 25 |
| ASP | CG | B | 386 | 22.5 | 6.1 | 11.7 | 32 |
| ASP | OD1 | B | 386 | 22.5 | 5.4 | 10.7 | 32 |
| ASP | OD2 | B | 386 | 22.6 | 5.8 | 12.9 | 33 |
| ASP | C | B | 386 | 21.3 | 7.4 | 9.2 | 26 |
| ASP | O | B | 386 | 20.2 | 7.1 | 9.7 | 26 |
| ARG | N | B | 387 | 21.5 | 7.2 | 7.9 | 19 |
| ARG | CA | B | 387 | 20.5 | 6.7 | 6.9 | 24 |
| ARG | CB | B | 387 | 21.1 | 6.7 | 5.5 | 25 |
| ARG | CG | B | 387 | 21.4 | 8.1 | 5.0 | 23 |
| ARG | CD | B | 387 | 22.1 | 8.0 | 3.6 | 27 |
| ARG | NE | B | 387 | 21.4 | 7.4 | 2.5 | 27 |
| ARG | CZ | B | 387 | 20.8 | 8.0 | 1.5 | 26 |
| ARG | NH1 | B | 387 | 20.8 | 9.3 | 1.4 | 21 |
| ARG | NH2 | B | 387 | 20.3 | 7.2 | 0.6 | 28 |
| ARG | C | B | 387 | 20.1 | 5.3 | 7.3 | 25 |
| ARG | O | B | 387 | 18.9 | 5.0 | 7.3 | 23 |
| ALA | N | B | 388 | 21.0 | 4.4 | 7.7 | 27 |
| ALA | CA | B | 388 | 20.6 | 3.1 | 8.1 | 29 |
| ALA | CB | B | 388 | 21.9 | 2.3 | 8.4 | 27 |
| ALA | C | B | 388 | 19.7 | 3.0 | 9.3 | 31 |
| ALA | O | B | 388 | 19.0 | 2.0 | 9.4 | 31 |
| ARG | N | B | 389 | 19.7 | 4.0 | 10.1 | 31 |
| ARG | CA | B | 389 | 18.8 | 3.9 | 11.3 | 31 |
| ARG | CB | B | 389 | 19.6 | 4.1 | 12.6 | 35 |
| ARG | CG | B | 389 | 20.7 | 3.0 | 12.7 | 37 |
| ARG | CD | B | 389 | 21.4 | 3.2 | 14.0 | 46 |
| ARG | NE | B | 389 | 22.4 | 2.1 | 14.2 | 54 |
| ARG | CZ | B | 389 | 23.5 | 2.0 | 13.4 | 57 |
| ARG | NH1 | B | 389 | 23.7 | 2.9 | 12.4 | 55 |
| ARG | NH2 | B | 389 | 24.4 | 1.1 | 13.6 | 58 |
| ARG | C | B | 389 | 17.7 | 5.0 | 11.2 | 31 |
| ARG | O | B | 389 | 17.0 | 5.2 | 12.1 | 32 |
| LYS | N | B | 390 | 17.7 | 5.7 | 10.1 | 31 |
| LYS | CA | B | 390 | 16.7 | 6.7 | 9.9 | 35 |
| LYS | CB | B | 390 | 15.3 | 6.0 | 9.8 | 41 |
| LYS | CG | B | 390 | 14.1 | 6.8 | 9.5 | 50 |
| LYS | CD | B | 390 | 12.9 | 5.9 | 9.4 | 58 |
| LYS | CE | B | 390 | 11.6 | 6.7 | 9.1 | 60 |
| LYS | NZ | B | 390 | 10.4 | 5.8 | 9.0 | 63 |
| LYS | C | B | 390 | 16.7 | 7.8 | 11.0 | 31 |
| LYS | O | B | 390 | 15.6 | 8.1 | 11.5 | 27 |
| ARG | N | B | 391 | 17.9 | 8.2 | 11.3 | 24 |
| ARG | CA | B | 391 | 18.0 | 9.2 | 12.4 | 24 |
| ARG | CB | B | 391 | 18.2 | 8.6 | 13.7 | 21 |
| ARG | CG | B | 391 | 19.5 | 7.8 | 13.8 | 21 |
| ARG | CD | B | 391 | 19.7 | 7.1 | 15.1 | 16 |
| ARG | NE | B | 391 | 21.0 | 6.4 | 15.1 | 22 |
| ARG | CZ | B | 391 | 21.6 | 5.9 | 16.2 | 21 |
| ARG | NH1 | B | 391 | 21.0 | 6.0 | 17.4 | 20 |
| ARG | NH2 | B | 391 | 22.7 | 5.3 | 16.1 | 21 |
| ARG | C | B | 391 | 19.1 | 10.2 | 12.1 | 23 |
| ARG | O | B | 391 | 20.1 | 9.9 | 11.4 | 20 |
| ILE | N | B | 392 | 19.0 | 11.4 | 12.7 | 26 |
| ILE | CA | B | 392 | 20.0 | 12.5 | 12.5 | 23 |
| ILE | CB | B | 392 | 19.4 | 13.8 | 12.0 | 24 |
| ILE | CG2 | B | 392 | 20.5 | 14.9 | 11.9 | 22 |
| ILE | CG1 | B | 392 | 18.8 | 13.6 | 10.6 | 24 |
| ILE | CD1 | B | 392 | 19.7 | 13.2 | 9.5 | 26 |
| ILE | C | B | 392 | 20.6 | 12.7 | 13.9 | 24 |
| ILE | O | B | 392 | 19.9 | 13.1 | 14.8 | 21 |
| GLY | N | B | 393 | 22.0 | 12.6 | 13.9 | 24 |
| GLY | CA | B | 393 | 22.7 | 12.9 | 15.2 | 23 |
| GLY | C | B | 393 | 23.4 | 14.2 | 15.3 | 26 |
| GLY | O | B | 393 | 24.0 | 14.7 | 14.3 | 24 |
| PHE | N | B | 394 | 23.3 | 14.8 | 16.5 | 26 |
| PHE | CA | B | 394 | 23.9 | 16.1 | 16.8 | 22 |
| PHE | CB | B | 394 | 22.9 | 17.2 | 17.0 | 19 |
| PHE | CG | B | 394 | 22.1 | 17.5 | 15.8 | 23 |
| PHE | CD1 | B | 394 | 20.9 | 16.8 | 15.5 | 23 |
| PHE | CD2 | B | 394 | 22.4 | 18.6 | 14.9 | 20 |
| PHE | CE1 | B | 394 | 20.1 | 17.1 | 14.4 | 18 |
| PHE | CE2 | B | 394 | 21.6 | 18.9 | 13.8 | 22 |
| PHE | CZ | B | 394 | 20.4 | 18.1 | 13.6 | 23 |
| PHE | C | B | 394 | 24.9 | 16.1 | 17.9 | 25 |
| PHE | O | B | 394 | 24.6 | 15.5 | 19.0 | 27 |
| ALA | N | B | 395 | 26.0 | 16.7 | 17.8 | 28 |
| ALA | CA | B | 395 | 27.1 | 16.8 | 18.8 | 25 |
| ALA | CB | B | 395 | 28.0 | 15.5 | 18.7 | 26 |
| ALA | C | B | 395 | 27.9 | 18.0 | 18.7 | 23 |
| ALA | O | B | 395 | 28.1 | 18.5 | 17.6 | 24 |
| VAL | N | B | 396 | 28.2 | 18.6 | 19.8 | 17 |
| VAL | CA | B | 396 | 29.0 | 19.9 | 19.8 | 18 |
| VAL | CB | B | 396 | 29.4 | 20.3 | 21.2 | 20 |
| VAL | CG1 | B | 396 | 30.3 | 21.6 | 21.2 | 17 |
| VAL | CG2 | B | 396 | 28.2 | 20.5 | 22.1 | 16 |
| VAL | C | B | 396 | 30.2 | 19.7 | 18.9 | 22 |
| VAL | O | B | 396 | 31.0 | 18.8 | 19.1 | 20 |
| SER | N | B | 397 | 30.3 | 20.6 | 17.9 | 22 |
| SER | CA | B | 397 | 31.4 | 20.6 | 17.0 | 24 |
| SER | CB | B | 397 | 31.1 | 21.4 | 15.7 | 22 |
| SER | OG | B | 397 | 32.2 | 21.3 | 14.8 | 26 |
| SER | C | B | 397 | 32.8 | 21.0 | 17.5 | 25 |
| SER | O | B | 397 | 32.9 | 22.1 | 18.1 | 27 |
| ALA | N | B | 398 | 33.8 | 20.2 | 17.3 | 22 |
| ALA | CA | B | 398 | 35.1 | 20.6 | 17.8 | 25 |
| ALA | CB | B | 398 | 36.1 | 19.4 | 17.6 | 24 |
| ALA | C | B | 398 | 35.7 | 21.8 | 17.1 | 27 |
| ALA | O | B | 398 | 36.8 | 22.3 | 17.4 | 33 |
| CYS | N | B | 399 | 35.0 | 22.3 | 16.1 | 30 |
| CYS | CA | B | 399 | 35.5 | 23.5 | 15.4 | 25 |
| CYS | C | B | 399 | 34.5 | 24.7 | 15.4 | 27 |
| CYS | O | B | 399 | 34.7 | 25.6 | 14.6 | 25 |
| CYS | CB | B | 399 | 35.8 | 23.2 | 13.9 | 29 |
| CYS | SG | B | 399 | 34.4 | 22.7 | 12.9 | 30 |
| HIS | N | B | 400 | 33.5 | 24.6 | 16.2 | 24 |
| HIS | CA | B | 400 | 32.5 | 25.7 | 16.1 | 23 |
| HIS | CB | B | 400 | 31.2 | 25.5 | 16.8 | 21 |
| HIS | CG | B | 400 | 31.3 | 25.4 | 18.3 | 21 |
| HIS | CD2 | B | 400 | 30.8 | 26.3 | 19.3 | 20 |
| HIS | ND1 | B | 400 | 31.8 | 24.3 | 19.0 | 21 |
| HIS | CE1 | B | 400 | 31.7 | 24.5 | 20.3 | 24 |
| HIS | NE2 | B | 400 | 31.1 | 25.7 | 20.5 | 22 |
| HIS | C | B | 400 | 33.2 | 27.0 | 16.8 | 23 |
| HIS | O | B | 400 | 34.0 | 26.9 | 17.7 | 22 |
| VAL | N | B | 401 | 32.8 | 28.1 | 16.2 | 23 |
| VAL | CA | B | 401 | 33.3 | 29.4 | 16.7 | 23 |
| VAL | CB | B | 401 | 33.2 | 30.5 | 15.7 | 26 |
| VAL | CG1 | B | 401 | 33.7 | 31.8 | 16.2 | 23 |
| VAL | CG2 | B | 401 | 33.9 | 30.1 | 14.4 | 22 |
| VAL | C | B | 401 | 32.6 | 29.8 | 18.0 | 27 |
| VAL | O | B | 401 | 31.4 | 29.8 | 18.1 | 27 |
| HIS | N | B | 402 | 33.4 | 30.1 | 19.0 | 28 |
| HIS | CA | B | 402 | 32.8 | 30.5 | 20.3 | 31 |
| HIS | CB | B | 402 | 32.3 | 29.3 | 21.2 | 32 |
| HIS | CG | B | 402 | 33.4 | 28.4 | 21.5 | 39 |
| HIS | CD2 | B | 402 | 34.1 | 28.2 | 22.7 | 40 |
| HIS | ND1 | B | 402 | 33.9 | 27.5 | 20.7 | 38 |
| HIS | CE1 | B | 402 | 34.9 | 26.8 | 21.3 | 40 |
| HIS | NE2 | B | 402 | 35.0 | 27.2 | 22.5 | 40 |
| HIS | C | B | 402 | 33.8 | 31.3 | 21.1 | 33 |
| HIS | O | B | 402 | 35.0 | 31.2 | 20.8 | 39 |
| ASP | N | B | 403 | 33.4 | 32.0 | 22.1 | 32 |
| ASP | CA | B | 403 | 34.3 | 32.8 | 23.0 | 30 |
| ASP | CB | B | 403 | 33.7 | 34.2 | 23.3 | 30 |
| ASP | CG | B | 403 | 32.3 | 34.0 | 23.9 | 35 |
| ASP | OD1 | B | 403 | 32.2 | 33.7 | 25.1 | 31 |
| ASP | OD2 | B | 403 | 31.3 | 34.3 | 23.3 | 36 |
| ASP | C | B | 403 | 34.5 | 32.0 | 24.2 | 32 |
| ASP | O | B | 403 | 34.1 | 30.8 | 24.3 | 28 |
| GLU | N | B | 404 | 35.2 | 32.6 | 25.2 | 34 |
| GLU | CA | B | 404 | 35.5 | 31.8 | 26.4 | 40 |
| GLU | CB | B | 404 | 36.8 | 32.3 | 27.1 | 48 |
| GLU | CG | B | 404 | 36.9 | 33.8 | 27.5 | 59 |
| GLU | CD | B | 404 | 36.7 | 34.8 | 26.3 | 66 |
| GLU | OE1 | B | 404 | 36.8 | 34.3 | 25.2 | 71 |
| GLU | OE2 | B | 404 | 36.6 | 36.0 | 26.5 | 67 |
| GLU | C | B | 404 | 34.4 | 31.8 | 27.5 | 35 |
| GLU | O | B | 404 | 34.5 | 31.1 | 28.5 | 37 |
| PHE | N | B | 405 | 33.3 | 32.5 | 27.2 | 28 |
| PHE | CA | B | 405 | 32.2 | 32.5 | 28.1 | 27 |
| PHE | CB | B | 405 | 31.8 | 34.0 | 28.4 | 29 |
| PHE | CG | B | 405 | 32.9 | 34.8 | 28.9 | 31 |
| PHE | CD1 | B | 405 | 33.4 | 35.8 | 28.1 | 33 |
| PHE | CD2 | B | 405 | 33.4 | 34.6 | 30.1 | 31 |
| PHE | CE1 | B | 405 | 34.5 | 36.6 | 28.5 | 34 |
| PHE | CE2 | B | 405 | 34.5 | 35.4 | 30.6 | 35 |
| PHE | CZ | B | 405 | 35.0 | 36.4 | 29.8 | 31 |
| PHE | C | B | 405 | 31.0 | 31.7 | 27.8 | 27 |
| PHE | O | B | 405 | 30.2 | 31.3 | 28.6 | 27 |
| ARG | N | B | 406 | 30.9 | 31.3 | 26.5 | 21 |
| ARG | CA | B | 406 | 29.8 | 30.5 | 26.0 | 24 |
| ARG | CB | B | 406 | 28.5 | 31.3 | 25.5 | 22 |
| ARG | CG | B | 406 | 27.9 | 32.1 | 26.6 | 24 |
| ARG | CD | B | 406 | 26.6 | 32.8 | 26.0 | 23 |
| ARG | NE | B | 406 | 26.9 | 33.7 | 24.8 | 22 |
| ARG | CZ | B | 406 | 26.1 | 34.6 | 24.4 | 22 |
| ARG | NH1 | B | 406 | 24.9 | 34.8 | 25.0 | 20 |
| ARG | NH2 | B | 406 | 26.4 | 35.3 | 23.3 | 18 |
| ARG | C | B | 406 | 30.2 | 29.5 | 24.9 | 23 |
| ARG | O | B | 406 | 31.1 | 29.8 | 24.1 | 25 |
| THR | N | B | 407 | 29.5 | 28.4 | 24.9 | 24 |
| THR | CA | B | 407 | 29.8 | 27.4 | 23.9 | 27 |
| THR | CB | B | 407 | 30.7 | 26.2 | 24.5 | 29 |
| THR | OG1 | B | 407 | 30.9 | 25.2 | 23.5 | 36 |
| THR | CG2 | B | 407 | 30.0 | 25.6 | 25.7 | 30 |
| THR | C | B | 407 | 28.5 | 26.8 | 23.3 | 23 |
| THR | O | B | 407 | 27.5 | 26.8 | 23.9 | 20 |
| ALA | N | B | 408 | 28.6 | 26.3 | 22.1 | 22 |
| ALA | CA | B | 408 | 27.4 | 25.7 | 21.4 | 24 |
| ALA | CB | B | 408 | 27.8 | 25.3 | 20.0 | 24 |
| ALA | C | B | 408 | 27.2 | 24.5 | 22.3 | 25 |
| ALA | O | B | 408 | 28.1 | 24.0 | 22.9 | 26 |
| ALA | N | B | 409 | 25.9 | 24.0 | 22.4 | 24 |
| ALA | CA | B | 409 | 25.6 | 22.9 | 23.2 | 24 |
| ALA | CB | B | 409 | 25.2 | 23.4 | 24.6 | 19 |
| ALA | C | B | 409 | 24.6 | 21.9 | 22.7 | 26 |
| ALA | O | B | 409 | 23.7 | 22.3 | 22.0 | 26 |
| VAL | N | B | 410 | 24.7 | 20.7 | 23.1 | 24 |
| VAL | CA | B | 410 | 23.8 | 19.6 | 22.7 | 24 |
| VAL | CB | B | 410 | 24.4 | 18.5 | 21.8 | 21 |
| VAL | CG1 | B | 410 | 23.3 | 17.5 | 21.4 | 22 |
| VAL | CG2 | B | 410 | 25.0 | 19.2 | 20.6 | 25 |
| VAL | C | B | 410 | 23.4 | 19.0 | 24.1 | 25 |
| VAL | O | B | 410 | 24.3 | 18.4 | 24.7 | 30 |
| GLU | N | B | 411 | 22.2 | 19.2 | 24.6 | 26 |
| GLU | CA | B | 411 | 21.8 | 18.7 | 25.9 | 28 |
| GLU | CB | B | 411 | 21.8 | 19.9 | 26.8 | 29 |
| GLU | CG | B | 411 | 23.2 | 20.7 | 26.9 | 38 |
| GLU | CD | B | 411 | 23.1 | 21.9 | 27.8 | 41 |
| GLU | OE1 | B | 411 | 23.9 | 22.0 | 28.7 | 46 |
| GLU | OE2 | B | 411 | 22.2 | 22.8 | 27.5 | 41 |
| GLU | C | B | 411 | 20.5 | 18.0 | 26.0 | 26 |
| GLU | O | B | 411 | 19.6 | 18.3 | 25.3 | 26 |
| GLY | N | B | 412 | 20.5 | 17.0 | 26.9 | 26 |
| GLY | CA | B | 412 | 19.3 | 16.2 | 27.1 | 24 |
| GLY | C | B | 412 | 19.4 | 15.2 | 28.3 | 27 |
| GLY | O | B | 412 | 20.4 | 15.1 | 28.9 | 25 |
| PRO | N | B | 413 | 18.3 | 14.5 | 28.6 | 28 |
| PRO | CD | B | 413 | 18.3 | 13.4 | 29.6 | 32 |
| PRO | CA | B | 413 | 17.0 | 14.5 | 28.0 | 31 |
| PRO | CB | B | 413 | 16.6 | 13.1 | 28.1 | 30 |
| PRO | CG | B | 413 | 16.9 | 12.8 | 29.5 | 30 |
| PRO | C | B | 413 | 16.0 | 15.4 | 28.7 | 31 |
| PRO | O | B | 413 | 16.1 | 15.8 | 29.8 | 31 |
| PHE | N | B | 414 | 14.9 | 15.8 | 27.9 | 34 |
| PHE | CA | B | 414 | 13.8 | 16.6 | 28.4 | 34 |
| PHE | CB | B | 414 | 13.8 | 18.0 | 27.7 | 29 |
| PHE | CG | B | 414 | 15.0 | 18.8 | 27.9 | 30 |
| PHE | CD1 | B | 414 | 16.0 | 18.7 | 26.9 | 30 |
| PHE | CD2 | B | 414 | 15.2 | 19.6 | 29.0 | 30 |
| PHE | CE1 | B | 414 | 17.2 | 19.4 | 27.1 | 26 |
| PHE | CE2 | B | 414 | 16.4 | 20.3 | 29.1 | 32 |
| PHE | CZ | B | 414 | 17.4 | 20.2 | 28.2 | 29 |
| PHE | C | B | 414 | 12.5 | 15.9 | 28.3 | 36 |
| PHE | O | B | 414 | 12.3 | 15.3 | 27.2 | 36 |
| VAL | N | B | 415 | 11.7 | 15.8 | 29.3 | 43 |
| VAL | CA | B | 415 | 10.5 | 15.1 | 29.3 | 48 |
| VAL | CB | B | 415 | 9.8 | 14.9 | 30.7 | 49 |
| VAL | CG1 | B | 415 | 8.5 | 14.2 | 30.5 | 48 |
| VAL | CG2 | B | 415 | 10.8 | 14.1 | 31.6 | 45 |
| VAL | C | B | 415 | 9.5 | 16.0 | 28.4 | 51 |
| VAL | O | B | 415 | 9.2 | 17.1 | 28.8 | 52 |
| THR | N | B | 416 | 9.1 | 15.4 | 27.3 | 56 |
| THR | CA | B | 416 | 8.3 | 16.2 | 26.4 | 59 |
| THR | CB | B | 416 | 9.0 | 16.7 | 25.2 | 59 |
| THR | OG1 | B | 416 | 10.2 | 17.5 | 25.6 | 57 |
| THR | CG2 | B | 416 | 8.2 | 17.6 | 24.3 | 61 |
| THR | C | B | 416 | 7.1 | 15.3 | 25.9 | 60 |
| THR | O | B | 416 | 7.3 | 14.2 | 25.4 | 59 |
| LEU | N | B | 417 | 5.9 | 15.8 | 26.1 | 62 |
| LEU | CA | B | 417 | 4.6 | 15.1 | 25.7 | 64 |
| LEU | CB | B | 417 | 3.6 | 15.1 | 26.8 | 64 |
| LEU | CG | B | 417 | 4.1 | 14.4 | 28.1 | 66 |
| LEU | CD1 | B | 417 | 3.1 | 14.6 | 29.2 | 68 |
| LEU | CD2 | B | 417 | 4.5 | 13.0 | 27.9 | 64 |
| LEU | C | B | 417 | 4.0 | 15.6 | 24.4 | 64 |
| LEU | O | B | 417 | 4.1 | 16.8 | 24.1 | 64 |
| ASP | N | B | 418 | 3.4 | 14.7 | 23.6 | 64 |
| ASP | CA | B | 418 | 2.7 | 15.1 | 22.4 | 65 |
| ASP | CB | B | 418 | 1.6 | 16.1 | 22.7 | 69 |
| ASP | CG | B | 418 | 0.5 | 15.6 | 23.6 | 74 |
| ASP | OD1 | B | 418 | −0.6 | 15.3 | 23.2 | 77 |
| ASP | OD2 | B | 418 | 0.8 | 15.4 | 24.8 | 75 |
| ASP | C | B | 418 | 3.6 | 15.5 | 21.3 | 61 |
| ASP | O | B | 418 | 3.2 | 16.1 | 20.3 | 57 |
| MET | N | B | 419 | 4.9 | 15.3 | 21.4 | 61 |
| MET | CA | B | 419 | 5.9 | 15.8 | 20.4 | 63 |
| MET | CB | B | 419 | 7.3 | 15.2 | 20.7 | 58 |
| MET | CG | B | 419 | 7.9 | 15.6 | 22.0 | 55 |
| MET | SD | B | 419 | 9.6 | 14.9 | 22.1 | 54 |
| MET | CE | B | 419 | 9.2 | 13.2 | 22.4 | 53 |
| MET | C | B | 419 | 5.5 | 15.4 | 19.0 | 65 |
| MET | O | B | 419 | 6.0 | 16.0 | 18.0 | 65 |
| GLU | N | B | 420 | 4.6 | 14.4 | 18.9 | 67 |
| GLU | CA | B | 420 | 4.2 | 14.0 | 17.6 | 68 |
| GLU | CB | B | 420 | 3.6 | 12.6 | 17.6 | 72 |
| GLU | CG | B | 420 | 3.2 | 11.9 | 16.3 | 80 |
| GLU | CD | B | 420 | 4.5 | 11.9 | 15.4 | 84 |
| GLU | OE1 | B | 420 | 5.0 | 10.8 | 15.1 | 85 |
| GLU | OE2 | B | 420 | 4.9 | 13.0 | 15.0 | 86 |
| GLU | C | B | 420 | 3.1 | 14.9 | 16.9 | 65 |
| GLU | O | B | 420 | 3.0 | 15.0 | 15.7 | 61 |
| ASP | N | B | 421 | 2.5 | 15.7 | 17.8 | 63 |
| ASP | CA | B | 421 | 1.5 | 16.7 | 17.4 | 61 |
| ASP | CB | B | 421 | 0.6 | 17.1 | 18.5 | 65 |
| ASP | CG | B | 421 | −0.2 | 15.9 | 19.1 | 71 |
| ASP | OD1 | B | 421 | −0.1 | 14.8 | 18.6 | 73 |
| ASP | OD2 | B | 421 | −0.8 | 16.1 | 20.1 | 73 |
| ASP | C | B | 421 | 2.2 | 17.9 | 16.8 | 57 |
| ASP | O | B | 421 | 1.6 | 18.9 | 16.3 | 55 |
| CYS | N | B | 422 | 3.5 | 17.9 | 16.9 | 51 |
| CYS | CA | B | 422 | 4.3 | 19.0 | 16.3 | 45 |
| CYS | C | B | 422 | 4.5 | 18.9 | 14.9 | 43 |
| CYS | O | B | 422 | 5.0 | 19.8 | 14.2 | 38 |
| CYS | CB | B | 422 | 5.6 | 19.1 | 17.1 | 42 |
| CYS | SG | B | 422 | 5.4 | 19.4 | 18.9 | 41 |
| GLY | N | B | 423 | 4.3 | 17.7 | 14.4 | 41 |
| GLY | CA | B | 423 | 4.5 | 17.4 | 12.9 | 41 |
| GLY | C | B | 423 | 3.3 | 17.9 | 12.1 | 43 |
| GLY | O | B | 423 | 2.1 | 17.8 | 12.6 | 47 |
| TYR | N | B | 424 | 3.6 | 18.5 | 11.0 | 44 |
| TYR | CA | B | 424 | 2.6 | 19.1 | 10.1 | 42 |
| TYR | CB | B | 424 | 3.1 | 20.4 | 9.5 | 44 |
| TYR | CG | B | 424 | 2.1 | 21.0 | 8.6 | 46 |
| TYR | CD1 | B | 424 | 1.0 | 21.8 | 9.0 | 47 |
| TYR | CE1 | B | 424 | 0.1 | 22.4 | 8.1 | 48 |
| TYR | CD2 | B | 424 | 2.2 | 20.9 | 7.2 | 47 |
| TYR | CE2 | B | 424 | 1.4 | 21.5 | 6.3 | 48 |
| TYR | CZ | B | 424 | 0.3 | 22.2 | 6.8 | 49 |
| TYR | OH | B | 424 | −0.6 | 22.8 | 5.9 | 52 |
| TYR | C | B | 424 | 2.3 | 18.1 | 9.0 | 47 |
| TYR | O | B | 424 | 3.2 | 17.5 | 8.4 | 47 |
| ASN | N | B | 425 | 1.0 | 17.9 | 8.7 | 48 |
| ASN | CA | B | 425 | 0.7 | 17.0 | 7.6 | 52 |
| ASN | CB | B | 425 | −0.4 | 16.0 | 8.1 | 51 |
| ASN | CG | B | 425 | 0.1 | 15.2 | 9.3 | 49 |
| ASN | OD1 | B | 425 | 0.4 | 14.0 | 9.2 | 51 |
| ASN | ND2 | B | 425 | 0.1 | 15.8 | 10.5 | 44 |
| ASN | C | B | 425 | 0.2 | 17.6 | 6.3 | 51 |
| ASN | O | B | 425 | −0.7 | 18.5 | 6.4 | 51 |
| CAL | Ca | M | 1 | 26.5 | −2.3 | 0.2 | 34 |
| CAL | Ca | M | 2 | −13.9 | −28.7 | 22.7 | 53 |
| SCH | N1 | S | 1 | 15.2 | −10.2 | 34.2 | 31 |
| SCH | C1 | S | 1 | 13.9 | −10.1 | 34.8 | 32 |
| SCH | C2 | S | 1 | 12.8 | −9.8 | 33.8 | 31 |
| SCH | O1 | S | 1 | 13.0 | −9.0 | 32.9 | 31 |
| SCH | C3 | S | 1 | 13.9 | −9.1 | 35.9 | 34 |
| SCH | C4 | S | 1 | 12.6 | −8.8 | 36.6 | 36 |
| SCH | C5 | S | 1 | 12.6 | −7.6 | 37.5 | 37 |
| SCH | O2 | S | 1 | 13.7 | −7.1 | 37.8 | 35 |
| SCH | O3 | S | 1 | 11.6 | −7.1 | 37.8 | 39 |
| SCH | N2 | S | 1 | 11.7 | −10.5 | 34.0 | 29 |
| SCH | C6 | S | 1 | 10.6 | −10.2 | 33.0 | 27 |
| SCH | C7 | S | 1 | 9.4 | −9.6 | 33.6 | 29 |
| SCH | O4 | S | 1 | 9.1 | −9.8 | 34.8 | 30 |
| SCH | C8 | S | 1 | 10.2 | −11.4 | 32.2 | 27 |
| SCH | C9 | S | 1 | 11.1 | −11.7 | 31.0 | 29 |
| SCH | C10 | S | 1 | 10.0 | −12.7 | 33.1 | 30 |
| SCH | N3 | S | 1 | 8.7 | −8.8 | 32.8 | 28 |
| SCH | C11 | S | 1 | 7.5 | −8.1 | 33.3 | 26 |
| SCH | C12 | S | 1 | 6.5 | −8.1 | 32.1 | 25 |
| SCH | O5 | S | 1 | 6.1 | −7.0 | 31.6 | 28 |
| SCH | C13 | S | 1 | 7.8 | −6.7 | 33.8 | 27 |
| SCH | C14 | S | 1 | 8.5 | −6.8 | 35.2 | 28 |
| SCH | O6 | S | 1 | 9.8 | −6.5 | 35.3 | 31 |
| SCH | N4 | S | 1 | 7.7 | −7.1 | 36.2 | 20 |
| SCH | C15 | S | 1 | 7.6 | −11.6 | 28.6 | 24 |
| SCH | C16 | S | 1 | 7.2 | −9.1 | 28.3 | 26 |
| SCH | C17 | S | 1 | 7.0 | −10.3 | 29.2 | 25 |
| SCH | C18 | S | 1 | 5.6 | −10.5 | 29.6 | 23 |
| SCH | N5 | S | 1 | 6.1 | −9.3 | 31.7 | 21 |
| SCH | C19 | S | 1 | 5.1 | −9.4 | 30.6 | 23 |
| SCH | C20 | S | 1 | 3.7 | −9.6 | 31.0 | 21 |
| SCH | O7 | S | 1 | 3.6 | −10.8 | 31.8 | 23 |
| SCH | C21 | S | 1 | 3.2 | −8.5 | 32.0 | 23 |
| SCH | C22 | S | 1 | 1.7 | −8.3 | 31.9 | 25 |
| SCH | C23 | S | 1 | 1.2 | −7.7 | 33.2 | 25 |
| SCH | C24 | S | 1 | 1.4 | −7.3 | 30.8 | 30 |
| SCH | O8 | S | 1 | 2.0 | −6.2 | 30.6 | 29 |
| SCH | N6 | S | 1 | 0.3 | −7.6 | 30.0 | 29 |
| SCH | C25 | S | 1 | −0.1 | −6.7 | 29.0 | 32 |
| SCH | C26 | S | 1 | −0.6 | −5.4 | 29.5 | 36 |
| SCH | O9 | S | 1 | −1.2 | −5.4 | 30.6 | 33 |
| SCH | C27 | S | 1 | −1.1 | −7.4 | 28.1 | 30 |
| SCH | N7 | S | 1 | −0.3 | −4.3 | 28.8 | 46 |
| SCH | C28 | S | 1 | −0.8 | −3.1 | 29.3 | 54 |
| SCH | C29 | S | 1 | −2.2 | −2.8 | 28.9 | 61 |
| SCH | O10 | S | 1 | −2.6 | −3.0 | 27.8 | 64 |
| SCH | C30 | S | 1 | 0.1 | −1.9 | 28.9 | 54 |
| SCH | C31 | S | 1 | 1.4 | −1.7 | 29.7 | 56 |
| SCH | C32 | S | 1 | 2.1 | −0.4 | 29.3 | 60 |
| SCH | O11 | S | 1 | 1.6 | 0.3 | 28.4 | 59 |
| SCH | O12 | S | 1 | 3.1 | −0.1 | 29.9 | 60 |
| SCH | N8 | S | 1 | −3.1 | −2.5 | 29.9 | 71 |
| SCH | C33 | S | 1 | −4.5 | −2.2 | 29.7 | 79 |
| SCH | C34 | S | 1 | −4.7 | −1.3 | 28.4 | 82 |
| SCH | O13 | S | 1 | −5.8 | −1.5 | 27.8 | 84 |
| SCH | C35 | S | 1 | −5.2 | −1.8 | 30.9 | 85 |
| SCH | C36 | S | 1 | −5.1 | −0.3 | 31.1 | 89 |
| SCH | C37 | S | 1 | −6.2 | 0.6 | 30.8 | 91 |
| SCH | C38 | S | 1 | −3.9 | 0.3 | 31.6 | 91 |
| SCH | C39 | S | 1 | −6.1 | 2.0 | 30.9 | 92 |
| SCH | C40 | S | 1 | −3.8 | 1.8 | 31.7 | 92 |
| SCH | C41 | S | 1 | −4.9 | 2.6 | 31.4 | 92 |
| SCH | O14 | S | 1 | −3.9 | −0.5 | 28.1 | 86 |
| SCH | N1 | S | 2 | 25.2 | 33.9 | −0.7 | 29 |
| SCH | C1 | S | 2 | 24.2 | 33.1 | 0.0 | 31 |
| SCH | C2 | S | 2 | 23.6 | 32.0 | −0.8 | 29 |
| SCH | O1 | S | 2 | 23.2 | 32.2 | −2.0 | 30 |
| SCH | C3 | S | 2 | 23.1 | 33.9 | 0.6 | 31 |
| SCH | C4 | S | 2 | 22.1 | 33.2 | 1.4 | 35 |
| SCH | C5 | S | 2 | 21.0 | 34.1 | 1.9 | 40 |
| SCH | O2 | S | 2 | 21.2 | 35.3 | 2.0 | 43 |
| SCH | O3 | S | 2 | 19.9 | 33.6 | 2.1 | 38 |
| SCH | N2 | S | 2 | 23.6 | 30.8 | −0.2 | 27 |
| SCH | C6 | S | 2 | 23.0 | 29.7 | −1.0 | 25 |
| SCH | C7 | S | 2 | 21.7 | 29.1 | −0.4 | 23 |
| SCH | O4 | S | 2 | 21.5 | 29.2 | 0.8 | 25 |
| SCH | C8 | S | 2 | 24.0 | 28.5 | −1.2 | 24 |
| SCH | C9 | S | 2 | 25.1 | 28.9 | −2.2 | 23 |
| SCH | C10 | S | 2 | 24.6 | 28.0 | 0.1 | 23 |
| SCH | N3 | S | 2 | 20.8 | 28.7 | −1.3 | 25 |
| SCH | C11 | S | 2 | 19.6 | 28.1 | −0.8 | 26 |
| SCH | C12 | S | 2 | 19.2 | 27.0 | −1.8 | 28 |
| SCH | O5 | S | 2 | 18.2 | 27.0 | −2.5 | 22 |
| SCH | C13 | S | 2 | 18.5 | 29.2 | −0.8 | 26 |
| SCH | C14 | S | 2 | 18.7 | 30.2 | 0.3 | 26 |
| SCH | O6 | S | 2 | 19.3 | 31.4 | −0.1 | 27 |
| SCH | N4 | S | 2 | 18.2 | 29.9 | 1.5 | 24 |
| SCH | C15 | S | 2 | 23.4 | 25.2 | −3.7 | 24 |
| SCH | C16 | S | 2 | 21.4 | 25.5 | −5.2 | 25 |
| SCH | C17 | S | 2 | 21.9 | 25.4 | −3.8 | 25 |
| SCH | C18 | S | 2 | 21.2 | 24.3 | −3.1 | 22 |
| SCH | N5 | S | 2 | 20.1 | 25.9 | −1.7 | 22 |
| SCH | C19 | S | 2 | 19.8 | 24.8 | −2.5 | 25 |
| SCH | C20 | S | 2 | 19.1 | 23.7 | −1.8 | 27 |
| SCH | O7 | S | 2 | 19.9 | 23.2 | −0.7 | 31 |
| SCH | C21 | S | 2 | 17.8 | 24.2 | −1.2 | 29 |
| SCH | C22 | S | 2 | 16.8 | 23.1 | −1.1 | 30 |
| SCH | C23 | S | 2 | 15.8 | 23.4 | 0.1 | 31 |
| SCH | C24 | S | 2 | 16.0 | 22.9 | −2.4 | 31 |
| SCH | O8 | S | 2 | 15.7 | 23.9 | −3.0 | 28 |
| SCH | N6 | S | 2 | 15.8 | 21.6 | −2.7 | 29 |
| SCH | C25 | S | 2 | 15.1 | 21.4 | −4.0 | 32 |
| SCH | C26 | S | 2 | 13.6 | 21.6 | −3.8 | 35 |
| SCH | O9 | S | 2 | 13.1 | 21.4 | −2.7 | 32 |
| SCH | C27 | S | 2 | 15.4 | 20.0 | −4.5 | 30 |
| SCH | N7 | S | 2 | 13.0 | 21.9 | −4.9 | 38 |
| SCH | C28 | S | 2 | 11.5 | 22.1 | −4.8 | 44 |
| SCH | C29 | S | 2 | 10.8 | 20.8 | −4.9 | 47 |
| SCH | O10 | S | 2 | 11.3 | 19.8 | −5.5 | 47 |
| SCH | C30 | S | 2 | 11.0 | 23.1 | −5.9 | 43 |
| SCH | C31 | S | 2 | 11.2 | 24.5 | −5.6 | 45 |
| SCH | C32 | S | 2 | 10.5 | 25.3 | −6.7 | 48 |
| SCH | O11 | S | 2 | 9.7 | 24.8 | −7.4 | 48 |
| SCH | O12 | S | 2 | 10.9 | 26.5 | −6.9 | 49 |
| SCH | N8 | S | 2 | 9.6 | 20.7 | −4.3 | 53 |
| SCH | C33 | S | 2 | 8.8 | 19.5 | −4.3 | 55 |
| SCH | C34 | S | 2 | 7.4 | 19.7 | −4.0 | 57 |
| SCH | O13 | S | 2 | 6.5 | 19.4 | −4.8 | 58 |
| SCH | C35 | S | 2 | 9.5 | 18.4 | −3.5 | 54 |
| SCH | C36 | S | 2 | 8.8 | 17.1 | −3.7 | 54 |
| SCH | C37 | S | 2 | 7.9 | 16.6 | −2.8 | 52 |
| SCH | C38 | S | 2 | 9.1 | 16.3 | −4.9 | 52 |
| SCH | C39 | S | 2 | 7.3 | 15.3 | −3.0 | 52 |
| SCH | C40 | S | 2 | 8.5 | 15.0 | −5.1 | 53 |
| SCH | C41 | S | 2 | 7.6 | 14.5 | −4.1 | 53 |
| SCH | O14 | S | 2 | 7.1 | 20.1 | −2.8 | 59 |
| WAT | OH2 | W | 1 | 22.7 | 10.9 | −2.7 | 16 |
| WAT | OH2 | W | 2 | 23.5 | 7.4 | −9.0 | 18 |
| WAT | OH2 | W | 3 | 20.5 | 18.5 | −6.7 | 17 |
| WAT | OH2 | W | 4 | 3.4 | −22.6 | 39.4 | 23 |
| WAT | OH2 | W | 5 | 8.2 | 26.5 | 22.1 | 24 |
| WAT | OH2 | W | 6 | 25.9 | 30.8 | 7.6 | 16 |
| WAT | OH2 | W | 7 | 16.6 | 38.5 | 8.0 | 18 |
| WAT | OH2 | W | 8 | 20.4 | 31.0 | 3.4 | 22 |
| WAT | OH2 | W | 9 | 9.5 | −9.0 | 37.8 | 20 |
| WAT | OH2 | W | 10 | 8.9 | 18.6 | 10.0 | 20 |
| WAT | OH2 | W | 11 | −6.1 | −18.0 | 18.4 | 30 |
| WAT | OH2 | W | 12 | 21.2 | 29.2 | −4.0 | 20 |
| WAT | OH2 | W | 13 | 37.9 | 17.2 | −1.4 | 26 |
| WAT | OH2 | W | 14 | 22.5 | 12.5 | −5.9 | 15 |
| WAT | OH2 | W | 15 | 28.7 | 34.0 | 22.5 | 21 |
| WAT | OH2 | W | 16 | 26.9 | 20.4 | 25.3 | 22 |
| WAT | OH2 | W | 17 | 17.5 | 17.9 | 3.5 | 18 |
| WAT | OH2 | W | 18 | 33.9 | 29.2 | −9.2 | 21 |
| WAT | OH2 | W | 19 | 37.8 | 14.3 | −2.5 | 26 |
| WAT | OH2 | W | 20 | 16.6 | 16.6 | −6.2 | 22 |
| WAT | OH2 | W | 21 | 28.1 | 20.4 | 10.3 | 17 |
| WAT | OH2 | W | 22 | 18.1 | −7.2 | 45.4 | 34 |
| WAT | OH2 | W | 23 | 4.1 | −12.6 | 64.4 | 24 |
| WAT | OH2 | W | 24 | −3.2 | −16.3 | 22.7 | 24 |
| WAT | OH2 | W | 25 | 16.3 | 26.7 | 29.2 | 23 |
| WAT | OH2 | W | 26 | 15.2 | 33.5 | 30.1 | 17 |
| WAT | OH2 | W | 27 | 21.1 | 13.7 | −11.5 | 19 |
| WAT | OH2 | W | 28 | 7.0 | 28.1 | 5.9 | 21 |
| WAT | OH2 | W | 29 | 12.1 | 23.4 | 4.7 | 30 |
| WAT | OH2 | W | 30 | 37.3 | 20.3 | −1.1 | 22 |
| WAT | OH2 | W | 31 | 33.7 | 27.1 | −11.3 | 24 |
| WAT | OH2 | W | 32 | 38.2 | 18.9 | −15.0 | 22 |
| WAT | OH2 | W | 33 | 18.3 | 50.3 | 7.5 | 22 |
| WAT | OH2 | W | 34 | 25.5 | 26.3 | 3.4 | 20 |
| WAT | OH2 | W | 35 | 24.1 | 13.4 | −20.4 | 34 |
| WAT | OH2 | W | 36 | 30.6 | 34.8 | 13.5 | 31 |
| WAT | OH2 | W | 37 | −5.4 | −12.9 | 21.0 | 29 |
| WAT | OH2 | W | 38 | 8.4 | −15.4 | 35.5 | 28 |
| WAT | OH2 | W | 39 | 9.6 | −7.8 | 30.3 | 30 |
| WAT | OH2 | W | 40 | 12.8 | −20.8 | 55.1 | 19 |
| WAT | OH2 | W | 41 | 37.4 | 25.1 | −9.7 | 28 |
| WAT | OH2 | W | 42 | 35.2 | 7.5 | −3.9 | 25 |
| WAT | OH2 | W | 43 | 32.1 | 5.1 | 12.2 | 25 |
| WAT | OH2 | W | 44 | 19.1 | 11.3 | −8.4 | 19 |
| WAT | OH2 | W | 45 | 21.6 | 11.9 | −9.4 | 17 |
| WAT | OH2 | W | 46 | 5.5 | 30.4 | 6.5 | 21 |
| WAT | OH2 | W | 47 | 14.2 | 28.1 | 30.4 | 20 |
| WAT | OH2 | W | 48 | 16.6 | −24.1 | 51.3 | 31 |
| WAT | OH2 | W | 49 | −5.0 | −18.0 | 25.4 | 29 |
| WAT | OH2 | W | 50 | 6.0 | −28.8 | 23.9 | 25 |
| WAT | OH2 | W | 51 | 20.6 | 9.7 | −1.3 | 24 |
| WAT | OH2 | W | 52 | 11.2 | −14.8 | 35.3 | 24 |
| WAT | OH2 | W | 53 | 30.6 | 28.1 | 2.8 | 28 |
| WAT | OH2 | W | 54 | −7.4 | −17.2 | 26.5 | 26 |
| WAT | OH2 | W | 55 | 12.4 | −16.8 | 36.7 | 27 |
| WAT | OH2 | W | 56 | 34.6 | 32.2 | −13.6 | 26 |
| WAT | OH2 | W | 57 | 26.7 | 29.0 | 2.7 | 22 |
| WAT | OH2 | W | 58 | 15.7 | −22.1 | 20.6 | 30 |
| WAT | OH2 | W | 59 | 6.1 | 19.1 | 9.8 | 28 |
| WAT | OH2 | W | 60 | 14.2 | −18.3 | 29.9 | 23 |
| WAT | OH2 | W | 61 | 33.3 | 34.9 | 14.2 | 29 |
| WAT | OH2 | W | 62 | 16.0 | −19.2 | 46.5 | 23 |
| WAT | OH2 | W | 63 | 12.6 | −18.3 | 34.6 | 30 |
| WAT | OH2 | W | 64 | 5.1 | −7.3 | 35.8 | 18 |
| WAT | OH2 | W | 65 | 23.8 | 10.3 | −8.8 | 23 |
| WAT | OH2 | W | 66 | 12.1 | −12.1 | 62.9 | 27 |
| WAT | OH2 | W | 67 | −1.8 | −27.9 | 49.3 | 25 |
| WAT | OH2 | W | 68 | 2.7 | −26.7 | 13.1 | 34 |
| WAT | OH2 | W | 69 | 8.5 | −18.3 | 60.2 | 31 |
| WAT | OH2 | W | 70 | 19.4 | −17.2 | 63.5 | 19 |
| WAT | OH2 | W | 71 | 5.7 | −25.0 | 41.6 | 20 |
| WAT | OH2 | W | 72 | 27.1 | 23.3 | 0.8 | 25 |
| WAT | OH2 | W | 73 | −3.2 | −12.6 | 34.6 | 22 |
| WAT | OH2 | W | 74 | 20.6 | 12.8 | −22.0 | 25 |
| WAT | OH2 | W | 75 | 8.5 | 37.0 | 20.2 | 32 |
| WAT | OH2 | W | 76 | 27.0 | −0.9 | 2.0 | 32 |
| WAT | OH2 | W | 77 | 4.6 | 30.9 | 9.2 | 29 |
| WAT | OH2 | W | 78 | 12.9 | 24.6 | 2.2 | 27 |
| WAT | OH2 | W | 79 | 10.3 | −26.1 | 27.0 | 27 |
| WAT | OH2 | W | 80 | 14.0 | 38.5 | 24.3 | 37 |
| WAT | OH2 | W | 81 | 17.4 | 34.1 | 3.0 | 34 |
| WAT | OH2 | W | 82 | 22.2 | 41.5 | 8.0 | 21 |
| WAT | OH2 | W | 83 | −1.1 | −14.1 | 63.1 | 28 |
| WAT | OH2 | W | 84 | 12.2 | −16.9 | 39.2 | 27 |
| WAT | OH2 | W | 85 | 12.8 | −6.9 | 21.6 | 30 |
| WAT | OH2 | W | 86 | 13.8 | −13.7 | 24.2 | 31 |
| WAT | OH2 | W | 87 | −2.7 | −9.5 | 25.3 | 32 |
| WAT | OH2 | W | 88 | 28.8 | 29.5 | 4.3 | 31 |
| WAT | OH2 | W | 89 | 28.6 | 22.6 | 25.2 | 33 |
| WAT | OH2 | W | 90 | 25.4 | 13.2 | −18.0 | 24 |
| WAT | OH2 | W | 91 | 14.6 | −24.7 | 14.4 | 44 |
| WAT | OH2 | W | 92 | 18.6 | −25.1 | 53.5 | 40 |
| WAT | OH2 | W | 93 | 30.8 | 19.0 | 24.4 | 24 |
| WAT | OH2 | W | 94 | −1.9 | −12.5 | 18.5 | 36 |
| WAT | OH2 | W | 95 | 15.7 | 47.8 | 14.2 | 26 |
| WAT | OH2 | W | 96 | 0.5 | −5.5 | 36.3 | 31 |
| WAT | OH2 | W | 97 | 1.0 | −11.5 | 24.4 | 26 |
| WAT | OH2 | W | 98 | 14.9 | −8.0 | 42.0 | 27 |
| WAT | OH2 | W | 99 | 9.4 | −12.1 | 63.0 | 19 |
| WAT | OH2 | W | 100 | 2.3 | −15.4 | 58.0 | 24 |
| WAT | OH2 | W | 101 | 14.5 | 26.6 | 1.2 | 24 |
| WAT | OH2 | W | 102 | 11.5 | 43.5 | 20.1 | 31 |
| WAT | OH2 | W | 103 | 11.4 | −10.2 | 15.0 | 31 |
| WAT | OH2 | W | 104 | 18.1 | 28.5 | 25.4 | 37 |
| WAT | OH2 | W | 105 | 12.3 | −3.8 | 45.3 | 28 |
| WAT | OH2 | W | 106 | 32.4 | 27.9 | −1.9 | 23 |
| WAT | OH2 | W | 107 | 11.7 | 34.2 | 23.3 | 36 |
| WAT | OH2 | W | 108 | 28.9 | 28.0 | −8.6 | 31 |
| WAT | OH2 | W | 109 | 1.0 | −30.0 | 51.7 | 33 |
| WAT | OH2 | W | 110 | 17.7 | 10.9 | 21.4 | 23 |
| WAT | OH2 | W | 111 | 30.7 | 21.6 | 12.6 | 16 |
| WAT | OH2 | W | 112 | 24.7 | 26.7 | −17.4 | 20 |
| WAT | OH2 | W | 113 | 31.1 | 28.0 | −10.4 | 33 |
| WAT | OH2 | W | 114 | 23.2 | 31.6 | −16.0 | 30 |
| WAT | OH2 | W | 115 | 23.4 | 33.4 | 26.9 | 48 |
| WAT | OH2 | W | 116 | 21.3 | 38.9 | 25.2 | 24 |
| WAT | OH2 | W | 117 | 14.0 | 37.7 | 6.4 | 29 |
| WAT | OH2 | W | 118 | 32.7 | 23.2 | 24.3 | 41 |
| WAT | OH2 | W | 119 | 18.7 | 45.3 | 19.3 | 24 |
| WAT | OH2 | W | 120 | 18.2 | −13.7 | 13.8 | 30 |
| WAT | OH2 | W | 121 | 40.5 | 17.3 | −14.8 | 30 |
| WAT | OH2 | W | 122 | 3.1 | −5.5 | 35.8 | 22 |
| WAT | OH2 | W | 123 | −0.9 | −16.2 | 61.2 | 27 |
| WAT | OH2 | W | 124 | 15.0 | −4.9 | 19.8 | 30 |
| WAT | OH2 | W | 125 | 27.0 | 30.8 | 10.2 | 26 |
| WAT | OH2 | W | 126 | 14.3 | −10.2 | 40.5 | 27 |
| WAT | OH2 | W | 127 | 29.8 | 17.5 | −21.7 | 26 |
| WAT | OH2 | W | 128 | −1.4 | −6.2 | 38.4 | 27 |
| WAT | OH2 | W | 129 | −0.3 | −5.8 | 62.9 | 25 |
| WAT | OH2 | W | 130 | −9.0 | −5.0 | 42.8 | 29 |
| WAT | OH2 | W | 131 | 17.1 | 7.5 | −1.5 | 38 |
| WAT | OH2 | W | 132 | 17.7 | 46.7 | 17.1 | 27 |
| WAT | OH2 | W | 133 | 21.4 | −21.8 | 18.7 | 32 |
| WAT | OH2 | W | 134 | 17.1 | 27.2 | 1.7 | 35 |
| WAT | OH2 | W | 135 | 28.0 | 16.8 | 22.2 | 21 |
| WAT | OH2 | W | 136 | 26.8 | −3.8 | −1.7 | 38 |
| WAT | OH2 | W | 137 | 27.6 | 15.8 | 26.1 | 30 |
| WAT | OH2 | W | 138 | 13.0 | 14.7 | 14.4 | 28 |
| WAT | OH2 | W | 139 | 17.4 | −6.4 | 57.5 | 34 |
| WAT | OH2 | W | 140 | 36.0 | 8.9 | −12.4 | 35 |
| WAT | OH2 | W | 141 | −10.3 | −13.9 | 44.2 | 28 |
| WAT | OH2 | W | 142 | 11.6 | −15.1 | 41.0 | 44 |
| WAT | OH2 | W | 143 | 8.1 | −28.5 | 25.7 | 36 |
| WAT | OH2 | W | 144 | 5.0 | 28.7 | 11.0 | 27 |
| WAT | OH2 | W | 145 | 15.9 | 5.5 | −7.1 | 40 |
| WAT | OH2 | W | 146 | 15.0 | −21.8 | 58.2 | 28 |
| WAT | OH2 | W | 147 | 18.3 | −21.1 | 58.0 | 35 |
| WAT | OH2 | W | 148 | 19.4 | 39.8 | 27.2 | 26 |
| WAT | OH2 | W | 149 | 34.6 | 35.2 | 16.6 | 26 |
| WAT | OH2 | W | 150 | 30.9 | 21.4 | 25.6 | 34 |
| WAT | OH2 | W | 151 | 34.1 | 34.5 | 19.3 | 40 |
| WAT | OH2 | W | 152 | 33.7 | 31.3 | −11.0 | 32 |
| WAT | OH2 | W | 153 | 36.7 | 22.4 | −18.8 | 29 |
| WAT | OH2 | W | 154 | 27.1 | 10.9 | −17.6 | 36 |
| WAT | OH2 | W | 155 | 10.5 | −22.7 | 44.9 | 28 |
| WAT | OH2 | W | 156 | 6.1 | 17.8 | 6.4 | 34 |
| WAT | OH2 | W | 157 | 30.9 | −0.7 | −8.8 | 29 |
| WAT | OH2 | W | 158 | 30.3 | 29.8 | 12.3 | 43 |
| WAT | OH2 | W | 159 | 28.5 | 30.3 | 6.9 | 22 |
| WAT | OH2 | W | 160 | 17.2 | 41.6 | 27.3 | 33 |
| WAT | OH2 | W | 161 | 0.5 | 1.9 | 43.7 | 26 |
| WAT | OH2 | W | 162 | 23.8 | 34.9 | 5.3 | 21 |
| WAT | OH2 | W | 163 | −3.6 | −17.3 | 19.2 | 28 |
| WAT | OH2 | W | 164 | 12.2 | −23.2 | 13.4 | 41 |
| WAT | OH2 | W | 165 | 25.0 | 6.5 | 14.1 | 36 |
| WAT | OH2 | W | 166 | 10.8 | −1.8 | 44.1 | 25 |
| WAT | OH2 | W | 167 | 12.6 | −13.5 | 60.7 | 35 |
| WAT | OH2 | W | 168 | 30.0 | 3.7 | −22.7 | 42 |
| WAT | OH2 | W | 169 | 30.8 | 31.6 | 6.8 | 36 |
| WAT | OH2 | W | 170 | 37.5 | 12.1 | −19.6 | 36 |
| WAT | OH2 | W | 171 | 7.5 | 23.8 | 25.5 | 32 |
| WAT | OH2 | W | 172 | 15.2 | −7.1 | 22.7 | 28 |
| WAT | OH2 | W | 173 | 11.2 | 30.1 | 1.2 | 39 |
| WAT | OH2 | W | 174 | 23.8 | 29.8 | −13.2 | 26 |
| WAT | OH2 | W | 175 | 18.0 | −23.6 | 48.7 | 40 |
| WAT | OH2 | W | 176 | 17.0 | −18.0 | 20.0 | 41 |
| WAT | OH2 | W | 177 | 17.4 | −21.5 | 46.9 | 46 |
| WAT | OH2 | W | 178 | 3.8 | −6.8 | 59.1 | 27 |
| WAT | OH2 | W | 179 | 15.1 | −3.5 | 22.2 | 38 |
| WAT | OH2 | W | 180 | 40.5 | 18.3 | −1.7 | 45 |
| WAT | OH2 | W | 181 | −14.0 | −20.7 | 50.6 | 37 |
| WAT | OH2 | W | 182 | 15.0 | 36.1 | 25.4 | 24 |
| WAT | OH2 | W | 183 | −2.7 | −3.3 | 17.9 | 34 |
| WAT | OH2 | W | 184 | 37.1 | 33.0 | −10.3 | 27 |
| WAT | OH2 | W | 185 | −1.1 | −28.3 | 52.0 | 31 |
| WAT | OH2 | W | 186 | 4.0 | −4.1 | 38.1 | 26 |
| WAT | OH2 | W | 187 | 8.6 | 10.1 | −8.1 | 44 |
| WAT | OH2 | W | 188 | 23.1 | 29.7 | −6.3 | 37 |
| WAT | OH2 | W | 189 | −4.6 | −7.2 | 63.1 | 35 |
| WAT | OH2 | W | 190 | 13.7 | 6.5 | 13.1 | 21 |
| WAT | OH2 | W | 191 | 30.7 | 27.8 | 14.2 | 26 |
| WAT | OH2 | W | 192 | 15.5 | −27.2 | 18.2 | 39 |
| WAT | OH2 | W | 193 | 26.8 | 24.5 | −18.1 | 33 |
| WAT | OH2 | W | 194 | 26.2 | 31.6 | 29.3 | 34 |
| WAT | OH2 | W | 195 | 32.9 | 49.6 | 17.8 | 41 |
| WAT | OH2 | W | 196 | 21.7 | 4.3 | 0.7 | 33 |
| WAT | OH2 | W | 197 | −7.1 | −9.0 | 58.5 | 36 |
| WAT | OH2 | W | 198 | 16.6 | 3.9 | 15.1 | 43 |
| WAT | OH2 | W | 199 | 27.9 | 48.9 | 15.6 | 32 |
| WAT | OH2 | W | 200 | −0.1 | −26.3 | 53.6 | 32 |
| WAT | OH2 | W | 201 | 6.8 | −9.5 | 60.9 | 29 |
| WAT | OH2 | W | 202 | 23.8 | 50.2 | 16.4 | 32 |
| WAT | OH2 | W | 203 | 11.2 | −8.5 | 28.2 | 34 |
| WAT | OH2 | W | 204 | 35.5 | 34.3 | −12.1 | 37 |
| WAT | OH2 | W | 205 | 13.3 | −6.3 | 40.5 | 35 |
| WAT | OH2 | W | 206 | 8.2 | −22.1 | 60.9 | 26 |
| WAT | OH2 | W | 207 | 13.4 | 28.7 | 2.6 | 34 |
| WAT | OH2 | W | 208 | 28.1 | 18.1 | 24.6 | 34 |
| WAT | OH2 | W | 209 | 19.7 | 5.4 | −14.5 | 41 |
| WAT | OH2 | W | 210 | 14.8 | −18.9 | 11.5 | 39 |
| WAT | OH2 | W | 211 | 20.3 | 32.4 | −2.8 | 45 |
| WAT | OH2 | W | 212 | 27.8 | −4.3 | 1.0 | 47 |
| WAT | OH2 | W | 213 | 36.8 | 7.9 | 15.1 | 51 |
| WAT | OH2 | W | 214 | 19.6 | −15.1 | 22.4 | 51 |
| WAT | OH2 | W | 215 | −8.1 | −7.6 | 42.1 | 28 |
| WAT | OH2 | W | 216 | 11.8 | −26.5 | 24.6 | 42 |
| WAT | OH2 | W | 217 | 36.5 | 18.2 | −17.4 | 38 |
| WAT | OH2 | W | 218 | −10.4 | −27.3 | 39.0 | 38 |
| WAT | OH2 | W | 219 | 7.6 | 35.2 | 26.6 | 34 |
| WAT | OH2 | W | 220 | 20.9 | 47.8 | 2.0 | 44 |
| WAT | OH2 | W | 221 | −13.3 | −28.9 | 25.1 | 39 |
| WAT | OH2 | W | 222 | 12.9 | −5.5 | 64.4 | 34 |
| WAT | OH2 | W | 223 | −4.3 | −7.3 | 56.1 | 40 |
| WAT | OH2 | W | 224 | −7.6 | −33.4 | 35.5 | 39 |
| WAT | OH2 | W | 225 | 7.1 | −17.2 | 31.9 | 21 |
| WAT | OH2 | W | 226 | 20.2 | 31.6 | 28.2 | 48 |
| WAT | OH2 | W | 227 | 12.6 | −9.3 | 64.3 | 38 |
| WAT | OH2 | W | 228 | −11.1 | −21.5 | 17.0 | 41 |
| WAT | OH2 | W | 229 | 1.2 | 29.5 | 17.8 | 38 |
| WAT | OH2 | W | 230 | 13.6 | 14.2 | −22.1 | 40 |
| WAT | OH2 | W | 231 | 20.4 | 9.8 | 21.5 | 31 |
| WAT | OH2 | W | 232 | 2.9 | 23.2 | 21.5 | 41 |
| WAT | OH2 | W | 233 | 2.7 | −26.9 | 53.8 | 30 |
| WAT | OH2 | W | 234 | 20.3 | 28.5 | 27.3 | 41 |
| WAT | OH2 | W | 235 | 38.7 | 20.9 | −3.7 | 37 |
| WAT | OH2 | W | 236 | 29.9 | 36.1 | 25.3 | 34 |
| WAT | OH2 | W | 237 | −13.4 | −27.6 | 35.9 | 43 |
| WAT | OH2 | W | 238 | 24.2 | −2.6 | 0.1 | 39 |
| WAT | OH2 | W | 239 | 10.4 | −5.0 | 39.0 | 33 |
| WAT | OH2 | W | 240 | 21.0 | 43.8 | 25.3 | 25 |
| WAT | OH2 | W | 241 | 26.3 | 6.5 | −24.8 | 37 |
| WAT | OH2 | W | 242 | 15.5 | 10.5 | 23.5 | 35 |
| WAT | OH2 | W | 243 | 31.0 | 3.0 | 9.8 | 55 |
| WAT | OH2 | W | 244 | 30.8 | 36.2 | −6.4 | 39 |
| WAT | OH2 | W | 245 | 19.0 | −9.2 | 27.0 | 35 |
| WAT | OH2 | W | 246 | 10.7 | 4.5 | 23.4 | 38 |
| WAT | OH2 | W | 247 | 7.2 | −33.2 | 28.9 | 34 |
| WAT | OH2 | W | 248 | 20.7 | −22.9 | 51.0 | 49 |
| WAT | OH2 | W | 249 | 25.0 | 0.0 | 6.8 | 30 |
| WAT | OH2 | W | 250 | 32.9 | 12.4 | 22.9 | 37 |
| WAT | OH2 | W | 251 | 13.4 | 45.3 | 21.0 | 34 |
| WAT | OH2 | W | 252 | 14.2 | −27.9 | 49.0 | 37 |
| WAT | OH2 | W | 253 | 19.6 | −11.0 | 37.5 | 36 |
| WAT | OH2 | W | 254 | 35.7 | 25.2 | 18.7 | 35 |
| WAT | OH2 | W | 255 | 40.1 | 20.0 | 1.0 | 35 |
| WAT | OH2 | W | 256 | 21.4 | −22.8 | 54.5 | 43 |
| WAT | OH2 | W | 257 | 9.2 | 35.5 | 23.8 | 41 |
| WAT | OH2 | W | 258 | 15.0 | 47.7 | 16.9 | 41 |
| WAT | OH2 | W | 259 | 15.8 | −16.2 | 22.3 | 36 |
| WAT | OH2 | W | 260 | 12.3 | −20.6 | 44.3 | 44 |
| WAT | OH2 | W | 261 | 37.6 | 25.9 | −18.4 | 50 |
| WAT | OH2 | W | 262 | 27.9 | 35.2 | −8.8 | 41 |
| WAT | OH2 | W | 263 | 36.7 | 27.9 | −1.8 | 42 |
| WAT | OH2 | W | 264 | −11.7 | −24.3 | 47.6 | 51 |
| WAT | OH2 | W | 265 | 4.6 | 2.6 | 41.8 | 39 |
| WAT | OH2 | W | 266 | 1.3 | −13.9 | 11.4 | 44 |
| WAT | OH2 | W | 267 | 21.2 | 1.7 | −1.5 | 41 |
| WAT | OH2 | W | 268 | 11.5 | 29.2 | −5.5 | 42 |
| WAT | OH2 | W | 269 | 12.1 | 17.6 | −6.5 | 45 |
| WAT | OH2 | W | 270 | −12.3 | −21.4 | 48.4 | 31 |
| WAT | OH2 | W | 271 | 5.8 | 37.3 | 19.1 | 29 |
| WAT | OH2 | W | 272 | 40.0 | 25.2 | −11.2 | 43 |
| WAT | OH2 | W | 273 | 30.2 | 38.7 | −11.4 | 26 |
| WAT | OH2 | W | 274 | −3.7 | −29.2 | 52.5 | 34 |
| WAT | OH2 | W | 275 | 11.7 | −21.1 | 11.7 | 40 |
| WAT | OH2 | W | 276 | 27.6 | 27.2 | −22.7 | 40 |
| WAT | OH2 | W | 277 | 41.0 | 15.0 | 3.2 | 32 |
| WAT | OH2 | W | 278 | 5.5 | 26.1 | 25.0 | 39 |
| WAT | OH2 | W | 279 | 40.0 | 20.8 | −16.0 | 35 |
| WAT | OH2 | W | 280 | 13.0 | 17.1 | −14.6 | 38 |
| WAT | OH2 | W | 281 | 18.3 | 4.6 | −11.4 | 41 |
| WAT | OH2 | W | 282 | −11.6 | −13.7 | 61.7 | 46 |
| WAT | OH2 | W | 283 | 16.1 | 46.2 | 20.2 | 31 |
| WAT | OH2 | W | 284 | 6.3 | 14.4 | 6.1 | 34 |
| WAT | OH2 | W | 285 | −9.4 | −4.4 | 39.1 | 34 |
| WAT | OH2 | W | 286 | 10.4 | 44.5 | 17.8 | 35 |
| WAT | OH2 | W | 287 | 10.7 | 12.8 | −7.1 | 35 |
| WAT | OH2 | W | 288 | 9.6 | −29.7 | 47.3 | 44 |
| WAT | OH2 | W | 289 | −9.7 | −29.8 | 42.8 | 35 |
| WAT | OH2 | W | 290 | 12.1 | −7.9 | 16.5 | 33 |
| WAT | OH2 | W | 291 | 21.1 | −18.9 | 61.8 | 53 |
| WAT | OH2 | W | 292 | 24.8 | 32.2 | −12.3 | 27 |
| WAT | OH2 | W | 293 | −0.7 | 0.2 | 41.7 | 36 |
| WAT | OH2 | W | 294 | −16.1 | −19.1 | 42.6 | 47 |
| WAT | OH2 | W | 295 | 31.0 | 39.5 | 24.1 | 22 |
| WAT | OH2 | W | 296 | 16.5 | −11.0 | 62.1 | 35 |
| WAT | OH2 | W | 297 | 4.5 | 36.0 | 14.6 | 43 |
| WAT | OH2 | W | 298 | 27.7 | 49.2 | 12.0 | 32 |
| WAT | OH2 | W | 299 | 14.3 | −26.7 | 38.5 | 42 |
| WAT | OH2 | W | 300 | 10.4 | 26.2 | −11.8 | 29 |
| WAT | OH2 | W | 301 | 40.6 | 27.8 | −8.2 | 49 |
| WAT | OH2 | W | 302 | 15.4 | −9.3 | 64.1 | 47 |
| WAT | OH2 | W | 303 | 23.0 | −21.2 | 50.9 | 36 |
| WAT | OH2 | W | 304 | 14.7 | −18.5 | 39.5 | 47 |
| WAT | OH2 | W | 305 | 5.8 | 38.1 | 16.4 | 39 |
| WAT | OH2 | W | 306 | 26.2 | 6.9 | 18.0 | 32 |
| WAT | OH2 | W | 307 | 12.3 | −1.6 | 59.3 | 32 |
| WAT | OH2 | W | 308 | 43.8 | 16.5 | −4.7 | 36 |
| WAT | OH2 | W | 309 | 24.8 | 15.8 | 26.4 | 35 |
| WAT | OH2 | W | 310 | 18.4 | 44.2 | 26.6 | 41 |
| WAT | OH2 | W | 311 | 39.7 | 14.0 | −19.9 | 34 |
| WAT | OH2 | W | 312 | 37.4 | 27.0 | 9.0 | 50 |
| WAT | OH2 | W | 313 | −3.5 | −14.5 | 19.8 | 34 |
| WAT | OH2 | W | 314 | −10.7 | −15.4 | 26.2 | 36 |
| WAT | OH2 | W | 315 | 9.1 | −29.9 | 17.7 | 45 |
| WAT | OH2 | W | 316 | 12.8 | 7.9 | −1.1 | 34 |
| WAT | OH2 | W | 317 | 27.9 | 36.5 | −5.6 | 48 |
| WAT | OH2 | W | 318 | 6.4 | 27.9 | 28.7 | 44 |
| WAT | OH2 | W | 319 | 23.3 | 16.6 | 28.5 | 40 |
| WAT | OH2 | W | 320 | 1.6 | 31.2 | 8.8 | 37 |
| WAT | OH2 | W | 321 | 5.0 | −17.0 | 61.3 | 53 |
| WAT | OH2 | W | 322 | 23.8 | 30.9 | 27.6 | 38 |
| WAT | OH2 | W | 323 | 0.8 | −12.0 | 9.5 | 51 |
| WAT | OH2 | W | 324 | 33.1 | 34.1 | −4.7 | 40 |
| WAT | OH2 | W | 325 | 23.5 | 3.6 | −18.2 | 33 |
| WAT | OH2 | W | 326 | 5.3 | −2.3 | 58.0 | 31 |
| WAT | OH2 | W | 327 | 17.1 | −21.9 | 28.6 | 54 |
| WAT | OH2 | W | 328 | 16.5 | −7.7 | 26.9 | 43 |
| WAT | OH2 | W | 329 | −5.0 | −10.2 | 63.3 | 52 |
| WAT | OH2 | W | 330 | 27.0 | 4.3 | 13.9 | 40 |
| WAT | OH2 | W | 331 | 42.4 | 17.2 | 1.8 | 61 |
| WAT | OH2 | W | 332 | 17.3 | −16.3 | 7.5 | 59 |
| WAT | OH2 | W | 333 | −4.5 | 1.6 | 54.6 | 39 |
| WAT | OH2 | W | 334 | 38.3 | 24.4 | 8.5 | 49 |
| WAT | OH2 | W | 335 | 20.1 | 44.6 | 28.9 | 39 |
| WAT | OH2 | W | 336 | 36.6 | 26.8 | 13.3 | 60 |
| WAT | OH2 | W | 337 | −2.6 | 2.3 | 40.5 | 49 |
| WAT | OH2 | W | 338 | 8.6 | −7.5 | 60.8 | 29 |
| WAT | OH2 | W | 339 | 2.8 | −17.9 | 59.1 | 30 |
| WAT | OH2 | W | 340 | 43.3 | 12.1 | 1.5 | 47 |
| WAT | OH2 | W | 341 | 8.9 | 40.5 | 14.0 | 45 |

The structural coordinates for the above-described BACE/OM-99-2 Complex crystal are set forth below.
“Res.” refers to the amino acid whose atomic coordinates have been determined.
“At.” refers to the atom, of the corresponding residue, whose coordinates have been determined.
“Ch.” refers to the molecule to which the corresponding residue belongs
“#” refers to the amino acid number of the corresponding residue.
“X”, “Y” and Z” refer to the crystallographically determined atomic position determined for each atom.
“B” refers to a thermal factor that measures movement of the atom around its atomic center.
“WAT” is the residue name corresponding to water molecules.
“SCH” is the residue name corresponding to the OM-99-2 inhibitors. There are two BACE molecules, called “A” and “B”, each with an OM-99-2 inhibitor called “1” and “2”.
“CAL” is the residue name corresponding to the two calcium ions.

The underlying structure of the β-secretase crystals was solved using molecular replacement as implemented in CNX (MSI Inc.). The molecular replacement protocol as described in the CNX manual was followed with minor modifications. The search model consisted of molecule A from the β-secretase structure deposited in the PDB (pdb code 1FKN). Analysis of the molecular replacement solution shows two molecules in the asymmetric unit. The active site of both molecules is open and not blocked by crystal contacts.

The present invention is not to be limited in scope by specific embodiments described herein. Indeed, various modifications of the invention, in addition to those described herein will become apparent to those skilled in the art from the foregoing description. Such modifications are intended to fall within the scope of the appended claims.

Patents, patent applications, publications, product descriptions and protocols are cited throughout this application; the disclosures of which are herein incorporated by reference in their entireties for all purposes.

We claim:

1. A crystal comprising a polypeptide selected from:
(a) a glycosylated, human β-secretase polypeptide characterized by structural coordinates comprising a root mean square deviation of conserved residue backbone atoms of less than about 1.5 Å when superimposed on backbone atoms described by structural coordinates of Table 2;
(b) a glycosylated, human β-secretase polypeptide complexed with 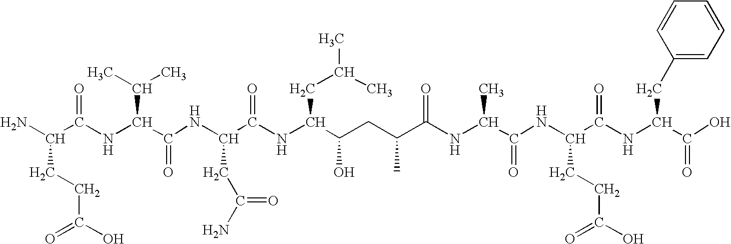
(OM-99-2) characterized by structural coordinates comprising a root mean square deviation of conserved residue backbone atoms of less than about 1.5 Å when superimposed on backbone atoms described by structural coordinates of Table 3; and
(c) a glycosylated, human β-secretase polypeptide which comprises a pyramidal structure.

2. A crystal of claim 1 wherein the root mean square deviation is less than about 1.0 Å.

3. A crystal of claim 2 wherein the root mean square deviation is less than about 0.5 Å.

4. A crystal of claim 3 wherein the root mean square deviation is less than about 0.1 Å.

5. A crystal of claim I comprising a polypeptide selected from:
(a) a glycosylated, human, β-secretase polypeptide comprising the amino acid sequence set forth in SEQ ID NO: 1;
(b) a glycosylated, human, β-secretase polypeptide comprising the amino acid sequence set forth in SEQ ID NO: 5 complexed with 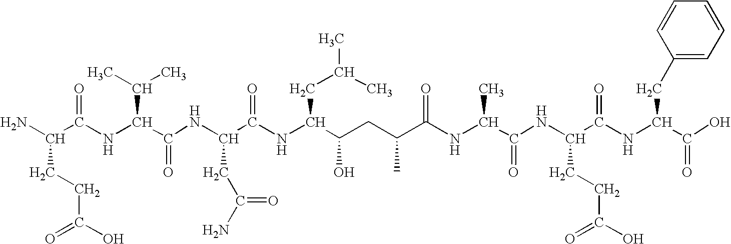
(OM-99-2); and
(c) a glycosylated, human β-secretase polypeptide comprising the amino acid sequence set forth in SEQ ID NO: 4 which crystal is characterized by a pyramidal structure.

6. A crystal of claim 1 comprising a polypeptide selected from:
(a) a glycosylated, human β-secretase polypeptide comprising the amino acid sequence set forth in SEQ ID NO: 1 characterized by structural coordinates of Table 2; and
(b) a glycosylated, human β-secretase polypeptide comprising the amino acid sequence set forth in SEQ ID NO: 5 complexed with 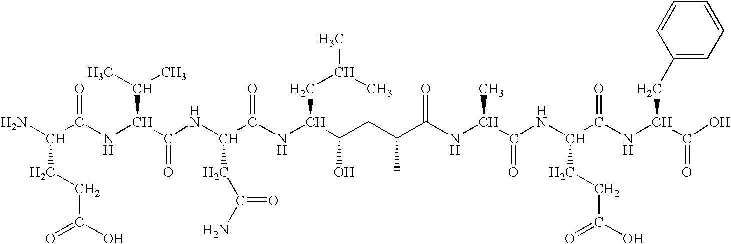
(OM-99-2) characterized by structural coordinates of Table 3.

7. A crystal of claim 1 which crystal is able to proteolytically cleave a peptide comprising the amino acid sequence KSEVNLDAEFRK (SEQ ID NO: 3).

8. A crystal of claim 1, wherein the β-secretase polypeptide comprises an active site in an open configuration.

9. A crystal of claim I which effectively diffracts x-rays for determination of structural coordinates of the polypeptide to a resolution greater than about 5 Å.

10. A computer for producing a three-dimensional representation of BACE characterized by the structural coordinates of Table 2 or BACE complexed with 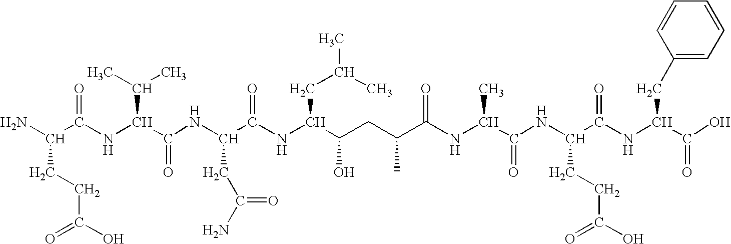
(OM-99-2) characterized by the structural coordinates of Table 3 or a three-dimensional representation of a homologue of said BACE or said BACE complexed with OM-99-2 wherein the homologue has a root mean square deviation from the backbone atoms of Table 2 or 3 of less than about 1.5 Å, wherein said computer comprises:
(a) a machine-readable data storage medium comprising a data storage material encoded with machine-readable data, wherein said data comprises the structure coordinates of Table 2 or 3;
(b) a working memory for storing instructions for processing said machine-readable data;
(c) a central-processing unit coupled to said working memory and to said machine-readable data storage medium for processing said machine readable data into said three-dimensional representation; and
(d) a display unit coupled to said central-processing unit for displaying said three-dimensional representation.

11. The computer of claim 10 wherein the root mean square deviation between the homologue and the structure coordinates set forth in Table 2 or 3 is less than about 1 Å.

12. The computer of claim 11 wherein the root mean square deviation between the homologue and the structure coordinates set forth in Table 2 or 3 is less than about 0.5 Å.

13. The computer of claim 12 wherein the root mean square deviation between the homologue and the structure coordinates set forth in Table 2 or 3 is less than about 0.1 Å.

14. The computer of claim 10 wherein the display unit is displaying the three dimensional representation.

15. A method for preparing crystalline, glycosylated, human β-secretase polypeptide, comprising subjecting a composition comprising a proBACE polypeptide (SEQ ID NO: 2) to a process selected from a microbatch method and a vapor diffusion method wherein said composition is at about pH 4.0.

16. The method of claim 15, wherein the proBACE polypeptide is first purified by a process selected from anion exchange chromatography, nickel chelate chromatography and gel filtration chromatography.

17. The method of claim 15, wherein the composition further comprises a member selected from a protein stabilizing agent, a salt and a precipitant.

18. A method for obtaining structural information concerning a molecule of unknown structure, comprising generating x-ray diffraction data from a crystallized form of the molecule, and applying crystallographic phases derived from at least a portion of structure coordinates set forth in Table 2 or 3 to said x-ray diffraction pattern to generate a three-dimensional electron density map of at least a portion of the molecule.
